(19) United States
(12) Patent Application Publication
(10) Pub. No.: US 2018/0353501 A1
(43) Pub. Date: Dec. 13, 2018

(54) MODULATORS OF PROTEOLYSIS AND ASSOCIATED METHODS OF USE

(71) Applicants: Arvinas, Inc., New Haven, CT (US); Yale University, New Haven, CT (US)

(72) Inventors: Andrew P. Crew, Guilford, CT (US); Hanqing Dong, Madison, CT (US); Jing Wang, Milford, CT (US); Craig M. Crews, New Haven, CT (US); Saul Jaime-Figueroa, Morris Plains, NJ (US)

(21) Appl. No.: 15/996,151

(22) Filed: Jun. 1, 2018

Related U.S. Application Data
(60) Provisional application No. 62/563,612, filed on Sep. 26, 2017.
(60) Provisional application No. 62/517,581, filed on Jun. 9, 2017.

(51) Int. Cl. A61K 31/47; A61K 45/06; A61K 47/54; A61K 31/427; A61K 31/454; A61K 31/5025; A61K 47/60

(52) U.S. Cl. CPC A61K 31/47; A61K 45/06; A61K 47/54; A61K 47/60; A61K 31/454; A61K 31/5025; A61K 31/427

(57) ABSTRACT

The present disclosure relates to bifunctional compounds, which find utility as modulators of c-Met and/or p38 (target protein). In particular, the present disclosure is directed to bifunctional compounds, which contain on one end a Von Hippel-Lindau, cereblon, Inhibitors of Apotosis Proteins or mouse double-minute homolog 2 ligand which binds to the respective E3 ubiquitin ligase and on the other end a moiety which binds the target protein, such that the target protein is placed in proximity to the ubiquitin ligase to effect degradation (and inhibition) of target protein. The present disclosure exhibits a broad range of pharmacological activities associated with degradation/inhibition of target protein. Diseases or disorders that result from aggregation or accumulation of the target protein are treated or prevented with compounds and compositions of the present disclosure.

### CROSS REFERENCE TO RELATED APPLICATIONS

The present application claims the benefit of U.S. Provisional Patent Application No. 62/517,581, filed 9 Jun. 2017 and U.S. Provisional Patent Application No. 62/563,612, filed 26 Sep. 2017, both of which are incorporated herein by reference in their entirety.

### INCORPORATION BY REFERENCE

U.S. patent application Ser. No. 15/230,354, filed on Aug. 5, 2016; and U.S. patent application Ser. No. 15/206,497 filed 11 Jul. 2016, published as U.S. Patent Application Publication No. 2017/0008904; and U.S. patent application Ser. No. 15/209,648 filed 13 Jul. 2016; and U.S. patent application Ser. No. 15/730,728, filed on Oct. 11, 2017; and U.S. patent application Ser. No. 14/686,640, filed on Apr. 14, 2015, published as U.S. Patent Application Publication No. 2015/0291562; and U.S. patent application Ser. No. 14/792,414, filed on Jul. 6, 2015, published as U.S. Patent Application Publication No. 2016/0058872; and U.S. patent application Ser. No. 14/371,956, filed on Jul. 11, 2014, published as U.S. Patent Application Publication No. 2014/0356322; and U.S. patent application Ser. No. 15/074,820, filed on Mar. 18, 2016, published as U.S. Patent Application Publication No. 2016/0272639; and U.S. patent application Ser. No., filed Jan. 31, 2018; and International Patent Application No. PCT/US2016/023258, filed Mar. 18, 2016, are incorporated herein by reference in their entirety. Furthermore, all references cited herein are incorporated by reference herein in their entirety.

### STATEMENT REGARDING FEDERALLY FUNDED RESEARCH

This invention was made with government support under grant number 1R44CA203199-01 by the National Institutes of Health. The government has certain rights in the invention.

### FIELD OF THE INVENTION

The description provides bifunctional compounds comprising a target protein binding moiety and a E3 ubiquitin ligase binding moiety, and associated methods of use. The bifunctional compounds are useful as modulators of targeted ubiquitination, especially with respect to c-Met and/or p38, which is degraded and/or otherwise inhibited by bifunctional compounds according to the present disclosure.

### BACKGROUND

Most small molecule drugs bind enzymes or receptors in tight and well-defined pockets. On the other hand, protein-protein interactions are notoriously difficult to target using small molecules due to their large contact surfaces and the shallow grooves or flat interfaces involved. E3 ubiquitin ligases (of which hundreds are known in humans) confer substrate specificity for ubiquitination, and therefore, are more attractive therapeutic targets than general proteasome inhibitors due to their specificity for certain protein substrates. The development of ligands of E3 ligases has proven challenging, in part due to the fact that they must disrupt protein-protein interactions. However, recent developments have provided specific ligands which bind to these ligases. For example, since the discovery of nutlins, the first small molecule E3 ligase inhibitors, additional compounds have been reported that target E3 ligases but the field remains underdeveloped. For example, since the discovery of Nutlins, the first small molecule E3 ligase mouse double minute 2 homolog (MDM2) inhibitors, additional compounds have been reported that target MDM2 (i.e., human double minute 2 or HDM2) E3 ligases (J. Di, et al. Current Cancer Drug Targets (2011), 11(8), 987-994).

Tumor suppressor gene p53 plays an important role in cell growth arrest and apoptosis in response to DNA damage or stress (A. Vazquez, et al. Nat. Rev. Drug. Dis. (2008), 7, 979-982), and inactivation of p53 has been suggested as one of the major pathway for tumor cell survival (A. J. Levine, et al. Nature (2000), 408, 307-310). In cancer patients, about 50% were found with p53 mutation (M. Hollstein, et al. Science (1991), 233, 49-53), while patients with wild type p53 were often found p53 down regulation by MDM2 through the protein-protein interaction of p53 and MDM2 (P. Chene, et al. Nat. Rev. Cancer (2003), 3, 102-109). Under normal cell condition without oncogenic stress signal, MDM2 keeps p53 at low concentration. In response to DNA damage or cellular stress, p53 level increases, and that also causes increase in MDM2 due to the feedback loop from p53/MDM2 auto regulatory system. In other words, p53 regulates MDM2 at the transcription level, and MDM2 regulates p53 at its activity level (A. J. Levine, et al. Genes Dev. (1993) 7, 1126-1132).

Several mechanisms can explain p53 down regulation by MDM2. First, MDM2 binds to N-terminal domain of p53 and blocks expression of p53-responsive genes (J. Momand, et al. Cell (1992), 69, 1237-1245). Second, MDM2 shuttles p53 from nucleus to cytoplasm to facilitate proteolytic degradation (J. Roth, et al. EMBO J. (1998), 17, 554-564). Lastly, MDM2 carries intrinsic E3 ligase activity of conjugating ubiquitin to p53 for degradation through ubiquitin-dependent 26s proteasome system (UPS) (Y. Haupt, et al. Nature (1997) 387, 296-299). As such, because MDM2 functions as E3 ligase, recruiting MDM2 to a disease causing protein and effectuating its ubiquitination and degradation is an approach of high interest for drug discovery.

One E3 ligase with exciting therapeutic potential is the von Hippel-Lindau (VHL) tumor suppressor, the substrate recognition subunit of the E3 ligase complex VCB, which also consists of elongins B and C, Cul2 and Rbx1. The primary substrate of VHL is Hypoxia Inducible Factor 1α (HIF-1α), a transcription factor that upregulates genes such as the pro-angiogenic growth factor VEGF and the red blood cell inducing cytokine erythropoietin in response to low oxygen levels. The first small molecule ligands of Von Hippel Lindau (VHL) to the substrate recognition subunit of the E3 ligase were generated, and crystal structures were obtained confirming that the compound mimics the binding mode of the transcription factor HIF-1α, the major substrate of VHL.

Cereblon is a protein that in humans is encoded by the CRBN gene. CRBN orthologs are highly conserved from plants to humans, which underscores its physiological importance. Cereblon forms an E3 ubiquitin ligase complex with damaged DNA binding protein 1 (DDB1), Cullin-4A (CUL4A), and regulator of cullins 1 (ROC1). This complex ubiquitinates a number of other proteins. Through a mechanism which has not been completely elucidated, cereblon ubquitination of target proteins results in increased levels of fibroblast growth factor 8 (FGF8) and fibroblast growth factor 10 (FGF10). FGF8 in turn regulates a number of developmental processes, such as limb and auditory vesicle formation. The net result is that this ubiquitin ligase complex is important for limb outgrowth in embryos. In the absence of cereblon, DDB1 forms a complex with DDB2 that functions as a DNA damage-binding protein.

Inhibitors of Apotosis Proteins (IAPs) are a protein family involved in suppressing apoptosis, i.e. cell death. The human IAP family includes 8 members, and numerous other organisms contain IAP homologs. IAPs contain an E3 ligase specific domain and baculoviral IAP repeat (BIR) domains that recognize substrates, and promote their ubiquitination. IAPs promote ubiquitination and can directly bind and inhibit caspases. Caspases are proteases (e.g. caspase-3, caspase-7 and caspace-9) that implement apoptosis. As such, through the binding of caspases, IAPs inhibit cell death. However, pro-apoptotic stimuli can result in the release of mitochondrial proteins DIABLO (also known as second mitrochondria-derived activator of caspases or SMAC) and HTRA2 (also known as Omi). Binding of DIABLO and HTRA2 appears to block IAP activity.

SMAC interacts with essentially all known IAPs including XIAP, c-IAP1, c-IAP2, NIL-IAP, Bruce, and survivin. The first four amino acids (AVPI) of mature SMAC bind to a portion of IAPs, which is believed to be essential for blocking the anti-apoptotic effects of IAPs.

Bifunctional compounds such as those that are described in U.S. Patent Application Publications 2015-0291562 and 2014-0356322 (incorporated herein by reference), function to recruit endogenous proteins to an E3 ubiquitin ligase for degradation. In particular, the publications describe bifunctional or proteolysis targeting chimeric (PROTAC) compounds, which find utility as modulators of targeted ubiquitination of a variety of polypeptides and other proteins, which are then degraded and/or otherwise inhibited by the bifunctional compounds.

Tyrosine-protein kinase Met (also known as, c-Met, MET, or Hepatocyte growth factor receptor [HGFR]) is a single pass tyrosine kinase receptor with tyrosine kinase activity and that plays a role in embryonic development, organogenesis, and wound healing. cMet is a known oncogene whose abnormal activation is associated with a poor prognosis in cancer patients (e.g., in kidney, liver, stomach, breast, and/or brain cancer). Furthermore, aberrant expression of MET has been implicated in secondary resistance to tyrosine kinase inhibitors (TKIs), e.g. gefitinib or erlotinib. As such, dysregulation of the MET signaling pathway is associated with increased invasiveness and progression of several cancers. For example, advanced hepatocellular carcinoma (HCC) and papillary renal cell cancer (RCC) have been associated with the dysregulation of the MET signaling pathway. MET has also been shown to promote a subset of gastric and non-small cell lung cancers through de novo mutations that lead to the skipping of exon 14. The exon 14 mutations results in drastic changes in cMet stability and activity. Subjects with an exon 14 mutation do not respond well to TKIs, such as crizotinib.

There also exists a lack of isoform-selective inhibitors for the p38 MAPK family, which have vast and varied roles in neurodegenerative diseases, cardiovascular disease, and cancer. Consisting of four members (α, β, γ, δ), the p38 MAPK serine/threonine kinases respond to environmental stress and cytokines and, individually, display differential tissue expression. Since its discovery in 1994, p38a has been the most well-studied isoform to date and, while dozens of inhibitors have been developed for clinical trials for the treatment of inflammatory diseases, none have demonstrated the efficacy and safety needed to receive FDA approval. Alternatively, p38δ is not only an understudied stress-sensing kinase with roles in cancer and diabetes, but its constrained ATP-binding pocket has made the discovery of potent, p38δ-selective inhibitors difficult. In fact, a recent study using a library of 178 commercially-available kinase inhibitors with selectivity for every major protein kinase subfamily showed that the p38δ kinase was altogether intractable to functional inhibition rendering it, effectively, “undruggable”.

An ongoing need exists in the art for effective treatments for (1) disease associated with overexpression or aggregation of cMet, and (2) disease associated with overexpression or aggregation of p38, such as p38α and/or p38δ. However, non-specific effects, and the inability to target and modulate cMET, p38α, and p38δ, remain as obstacles to the development of effective treatments for diseases associated with the overexpression or aggregation of each of these proteins. As such, small-molecule therapeutic agents that target cMet p38α, and/or p38δ and that leverage or potentiate VHL's, cereblon's, MDM2's, and IAPs' substrate specificity would be very useful.

### SUMMARY

The present disclosure describes bifunctional compounds which function to recruit endogenous proteins to an E3 ubiquitin ligase for degradation, and methods of using the same. In particular, the present disclosure provides bifunctional or proteolysis targeting chimeric (PROTAC) compounds, which find utility as modulators of targeted ubiquitination of cMet and/or p38, which are then degraded and/or otherwise inhibited by the bifunctional compounds as described herein. An advantage of the compounds provided herein is that a broad range of pharmacological activities is possible, consistent with the degradation/inhibition of targeted polypeptides from virtually any protein class or family. In addition, the description provides methods of using an effective amount of the compounds as described herein for the treatment or amelioration of a disease condition, such as cancer, e.g., gastric, non-small cell lung cancer, advanced hepatocellular carcinoma (HCC), and/or papillary renal cell cancer (RCC).

As such, in one aspect the disclosure provides bifunctional or PROTAC compounds, which comprise an E3 ubiquitin ligase binding moiety (i.e., a ligand for an E3 ubiquitin ligase or “ULM” group), and a moiety that binds a target protein (i.e., a protein/polypeptide targeting ligand or “PTM” group) such that the target protein/polypeptide is placed in proximity to the ubiquitin ligase to effect degradation (and inhibition) of that protein. In a preferred embodiment, the ULM (ubiquitination ligase modulator) can be Von Hippel-Lindau E3 ubiquitin ligase (VHL) binding moiety (VLM), or a cereblon E3 ubiquitin ligase binding moiety (CLM), or a mouse double minute 2 homolog (MDM2) E3 ubiquitin ligase binding moiety (MLM), or an IAP E3 ubiquitin ligase binding moiety (i.e., a “ILM”). For example, the structure of the bifunctional compound can be depicted as:

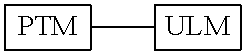

The respective positions of the PTM and ULM moieties (e.g., VLM, CLM, MLM or ILM) as well as their number as illustrated herein is provided by way of example only and is not intended to limit the compounds in any way. As would be understood by the skilled artisan, the bifunctional compounds as described herein can be synthesized such that the number and position of the respective functional moieties can be varied as desired.

In certain embodiments, the bifunctional compound further comprises a chemical linker (“L”). In this example, the structure of the bifunctional compound can be depicted as:

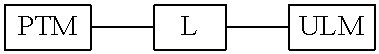

where PTM is a protein/polypeptide targeting moiety, L is a linker, e.g., a bond or a chemical group coupling PTM to ULM, and ULM is a IAP E3 ubiquitin ligase binding moiety, or a Von Hippel-Lindau E3 ubiquitin ligase (VHL) binding moiety (VLM), or a cereblon E3 ubiquitin ligase binding moiety (CLM), or a mouse double minute 2 homolog (MDM2) E3 ubiquitin ligase binding moiety (MLM).

For example, the structure of the bifunctional compound can be depicted as:

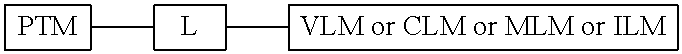

wherein: PTM is a protein/polypeptide targeting moiety; “L” is a linker (e.g. a bond or a chemical linker group) coupling the PTM and at least one of VLM, CLM, MLM, ILM, or a combination thereof; VLM is Von Hippel-Lindau E3 ubiquitin ligase binding moiety that binds to VHL E3 ligase; CLM is cereblon E3 ubiquitin ligase binding moiety that binds to cereblon; MLM is an MDM2 E3 ubiquitin ligase binding moiety that binds MDM2; and ILM is a IAP binding moiety that binds to IAP.

In certain preferred embodiments, the ILM is an AVPI tetrapeptide fragment. As such, in certain additional embodiments, the ILM of the bifunctional compound comprises the amino acids alanine (A), valine (V), proline (P), and isoleucine (I) or their unnatural mimetics, respectively. In additional embodiments, the amino acids of the AVPI tetrapeptide fragment are connected to each other through amide bonds (i.e., —C(O)NH— or —NHC(O)—).

In certain embodiments, the compounds as described herein comprise multiple independently selected ULMs, multiple PTMs, multiple chemical linkers or a combination thereof.

In certain embodiments, ILM comprises chemical moieties such as those described herein.

In additional embodiments, VLM can be hydroxyproline or a derivative thereof. Furthermore, other contemplated VLMs are included in U.S. Patent Application Publication No. 2014/03022523, which as discussed above, is incorporated herein in its entirety.

In an embodiment, the CLM comprises a chemical group derived from an imide, a thioimide, an amide, or a thioamide. In a particular embodiment, the chemical group is a phthalimido group, or an analog or derivative thereof. In a certain embodiment, the CLM is thalidomide, lenalidomide, pomalidomide, analogs thereof, isosteres thereof, or derivatives thereof. Other contemplated CLMs are described in U.S. Patent Application Publication No. 2015/0291562, which is incorporated herein in its entirety.

In certain embodiments, MLM can be nutlin or a derivative thereof. Furthermore, other contemplated MLMs are included in U.S. Patent Application Publication No. 2017/0008904 (U.S. Ser. No. 15/206,497, filed 11 Jul. 2016), which as discussed above, is incorporated herein in its entirety. In certain additional embodiments, the MLM of the bifunctional compound comprises chemical moieties such as substituted imidazolines, substituted spiro-indolinones, substituted pyrrolidines, substituted piperidinones, substituted morpholinones, substituted pyrrolopyrimidines, substituted imidazolopyridines, substituted thiazoloimidazoline, substituted pyrrolopyrrolidinones, and substituted isoquinolinones.

In additional embodiments, the MLM comprises the core structures mentioned above with adjacent bis-aryl substitutions positioned as cis- or trans-configurations.

In certain embodiments, “L” is a bond. In additional embodiments, the linker “L” is a connector with a linear non-hydrogen atom number in the range of 1 to 20. The connector “L” can contain, but not limited to the functional groups such as ether, amide, alkane, alkene, alkyne, ketone, hydroxyl, carboxylic acid, thioether, sulfoxide, and sulfone. The linker can contain aromatic, heteroaromatic, cyclic, bicyclic and tricyclic moieties. Substitution with halogen, such as Cl, F, Br and I can be included in the linker. In the case of fluorine substitution, single or multiple fluorines can be included.

In other embodiments, the linker is

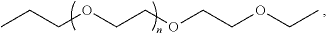

wherein n is an integer from 0 to 10. In yet other embodiments, the linker is

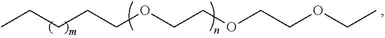

wherein n is an integer from 0 to 10, and m is an integer from 2 to 10. In still other embodiments, the linker is

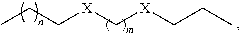

wherein n is an integer from 0 to 10, m is an integer from 0 to 10, and X is independently O or CH₂.

In certain embodiments, VLM is a derivative of trans-3-hydroxyproline, where both nitrogen and carboxylic acid in trans-3-hydroxyproline are functionalized as amides.

In certain embodiments, CLM is a derivative of piperidine-2,6-dione, where piperidine-2,6-dione can be substituted at the 3-position, and the 3-substitution can be bicyclic hetero-aromatics with the linkage as C—N bond or C—C bond. Examples of CLM can be, but not limited to, pomalidomide, lenalidomide and thalidomide and their derivatives.

In an additional aspect, the description provides therapeutic compositions comprising an effective amount of a compound as described herein or salt form thereof, and a pharmaceutically acceptable carrier. The therapeutic compositions modulate protein degradation and/or inhibition in a patient or subject, for example, an animal such as a human, and can be used for treating or ameliorating disease states or conditions which are modulated through the degraded/inhibited protein. In certain embodiments, the therapeutic compositions as described herein may be used to effectuate the degradation of proteins of interest (c-Met) for the treatment or amelioration of a disease, e.g., cancer. In yet another aspect, the present disclosure provides a method of ubiquitinating/degrading a target protein in a cell. In certain embodiments, the method comprises administering a bifunctional compound as described herein comprising an ILM and a PTM, a PTM and a VLM, or a PTM and a CLM, or a PTM and a MLM, preferably linked through a linker moiety, as otherwise described herein, wherein the VLM/ILM/CLM/MLM is coupled to the PTM through a linker to target protein that binds to PTM for degradation. Similarly, the PTM can be coupled to VLM or CLM or MLM or ILM through a linker to target a protein or polypeptide for degradation. Degradation of the target protein will occur when the target protein is placed in proximity to the E3 ubiquitin ligase, thus resulting in degradation/inhibition of the effects of the target protein and the control of protein levels. The control of protein levels afforded by the present disclosure provides treatment of a disease state or condition, which is modulated through the target protein by lowering the level of that protein in the cells of a patient.

In still another aspect, the description provides methods for treating or ameliorating a disease, disorder or symptom thereof in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable carrier, wherein the composition is effective for treating or ameliorating the disease or disorder or symptom thereof in the subject.

In another aspect, the description provides methods for identifying the effects of the degradation of proteins of interest in a biological system using compounds according to the present disclosure.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional aspects and embodiments are expressly included within the scope of the present disclosure. The publications and other materials used herein to illuminate the background of the invention, and in particular cases, to provide additional details respecting the practice, are incorporated by reference.

### BRIEF DESCRIPTION OF THE DRAWINGS

The accompanying drawings, which are incorporated into and form a part of the specification, illustrate several embodiments of the present disclosure and, together with the description, serve to explain the principles of the invention. The drawings are only for the purpose of illustrating an embodiment of the invention and are not to be construed as limiting the invention. Further objects, features and advantages of the invention will become apparent from the following detailed description taken in conjunction with the accompanying figures showing illustrative embodiments of the invention, in which:

FIGS. 1A and 1B. Illustration of general principle for PROTAC function. (A) Exemplary PROTACs comprise a protein targeting moiety (PTM; darkly shaded rectangle), a ubiquitin ligase binding moiety (ULM; lightly shaded triangle), and optionally a linker moiety (L; black line) coupling or tethering the PTM to the ULM. (B) Illustrates the functional use of the PROTACs as described herein. Briefly, the ULM recognizes and binds to a specific E3 ubiquitin ligase, and the PTM binds and recruits a target protein bringing it into close proximity to the E3 ubiquitin ligase. Typically, the E3 ubiquitin ligase is complexed with an E2 ubiquitin-conjugating protein, and either alone or via the E2 protein catalyzes attachment of ubiquitin (dark circles) to a lysine on the target protein via an isopeptide bond. The poly-ubiquitinated protein (far right) is then targeted for degradation by the proteosomal machinery of the cell.

FIG. 2. Table 1 includes target protein degradation data for exemplary PROTACS.

FIGS. 3A and 3B. Illustrate the dose response of Example 1 (3A) and Example 6 (3B) PROTACS.

FIG. 4. Illustrates the degradation of Met by exemplary VHL (Examples 1 and 6) and cereblon (Examples 2 and 8) PROTACS.

FIG. 5. Illustrates the pharmacokinetics for intraperitoneally and intravenously injected PROTAC Example 1 in CD1 mice.

FIG. 6. Table 4 includes pharmacokinetic data for Example 1 in CD1 mice.

FIG. 7. Illustrates the pharmacokinetics of intraperitoneally and intravenously injected PROTAC Example 2 in CD1 mice.

FIG. 8. Illustrates the pharmacokinetic data for Example 2 in CD1 mice.

FIGS. 9A, 9B, and 9C. Foretinib-based PROTACs recruit VHL using two different linkage vectors resulting in isoform-selective degradation. (A) Crystal structure (PDB: 4W9H) of VHL-recruiting ligand in the HIF-1a binding pocket. VHL is rendered as a tan surface with the small molecule ligand visualized in stick representation and colored by atom (grey carbon atoms, red oxygen atoms, blue nitrogen atoms, and yellow sulfur atoms), with the exception of two carbon atoms colored in purple. These purple carbons (and the corresponding arrows) represent the structure-guided linker attachment points used in subsequent PROTAC design (see FIG. 10). (B) Structures of the two VHL-recruiting ligands used in this study. Top: VHL-recruiting ligand with an amide attachment vector (shown with an R). Bottom: VHL-recruiting ligand with a phenyl attachment vector (shown with an R). (C) exemplary PROTAC 50 (13-atom linker, amide attachment) selectively degrades p38α, whereas exemplary PROTAC 46 (10-atom linker, phenyl attachment) selectively degrades p38δ in MDA-MB-231 cells. Summary table of the respective DC50 and Dmax values per PROTAC are provided. See FIG. 10 for the structures of all of the PROTACs screened in this study with their corresponding western blots (FIGS. 11A and 11B) and summary table (Table 13). See FIGS. 12A, 12B, and 12C for further investigation into the activity of exemplary PROTACs on p38, ERK, and JNK MAPK families.

FIGS. 10A and 10B. Structures of exemplary PROTACs with the calculated total polar surface area (tPSA) and CLogP values for each structure. The eight PROTACs are divided into two groups—(A) “amide series” and (B) “phenyl series” PROTACs—based on the differences in their VHL-recruiting moieties. Each series contains four PROTACs based on a foretinib PTM and linker lengths of 10, 11, 12, and 13 atoms.

FIGS. 11A and 11B. Survey of p38 isoform degradation with foretinib-based VHL PROTACs (related to FIGS. 9A-9C and Table 13). (A) Western blots of amide series PROTACs tested in increasing concentrations (0.025, 0.100, 0.250, 1.0, 5.0 μM) on MDA-MB-231 cells, in duplicate. (B) Same as in (A) but with the phenyl series PROTACs. Summary table of the DC50 (concentration at which half-maximal degradation is achieved) and Dmax (maximum percentage of degradation achieved) is reported, per exemplary PROTAC, in Table 13.

FIGS. 12A, 12B, and 12C. Characterization of MAPK family degradation selectivity (related to FIGS. 9A, 9B, 9C, 13A, 13B, 13C, and 13D). (A) p38 MAPK family members (α, β, δ, γ) were aligned using the T-Coffee multiple sequence alignment server. Output results were then colored according to conservation (dark turquoise) and identity (lighter shades of turquoise) in Jalview. (B) Table summarizing pairwise sequence alignments (obtained in Jalview) and structural alignments for Ca atoms (obtained in PyMOL) using the doublyphosphorylated (pTGpY) active conformations of p38δ (PDB: 4MYG), p38α (PDB: 3PY3), and p38γ (PDB: 1CM8). No active conformation crystal structure of p38β currently exists. (C) Western blots characterizing the lack of degradation of the remaining p38 MAPK (p38β, p38γ), ERK MAPK (ERK1, ERK2), and JNK MAPK (JNK1, JNK2) family members with increasing concentrations of exemplary compound 50 and 46 in MDA-MB-231 cells. MAPK families are also divided based on their phosphorylation lip motif, (pTxpY). Note: JNK1 and JNK2 are expressed as two alternatively-spliced forms. JNK3 was not expressed in these cells.

FIGS. 13A, 13B, 13C, and 13D. Exemplary compounds 50 and 46 degrade p38 isoforms in a manner that is rapid, sustained, and proteasome-dependent. (A) MDA-MB-231 cells were either pre-treated or not with the proteasome inhibitor epoxomicin (1 μM) or the neddylation inhibitor MLN4924 (1 μM) for 30 minutes and subsequently treated with either vehicle (DMSO), exemplary compound 50 (250 nM), or exemplary compound 46 (250 nM) for 6 hours. (B) Quantitative real-time PCR was performed after 24 hours of treatment with either vehicle (DMSO), exemplary compound 50 (250 nM), or exemplary compound 46 (250 nM) in MDAMB-231 cells. The dotted line represents DMSO-treated conditions standardized to a value of 1.0 and mRNA abundance (per treatment group) is represented as a foldchange relative to that value. Data is based on biological duplicates and is normalized to beta-tubulin. (C) Cycloheximide (CHX) chase assay. MDA-MB-231 cells were pre-treated with 100 μg/mL CHX for 1 hour prior to treating for the indicated times with either vehicle (DMSO), exemplary compound 50 (250 nM), or exemplary compound 46 (250 nM). Tubulin-normalized p38α and p38δ abundance values are reported beneath individual lanes. (D) MDA-MB231 cells were treated with either vehicle (DMSO), exemplary compound 50 (250 nM), or exemplary compound 46 (250 nM) for 24 hours before re-plating onto new plastic in fresh medium for an additional 24, 48, or 72 hours (“washout” conditions).

FIGS. 14A, 14B, and 14C. p38α selectivity is discriminated by ternary complex formation. (A) Only p38α incubated in the presence of exemplary compound 50 can be immunoprecipitated with GST-tagged VHL/EloB/EloC (VBC). Immobilized VBC was used as a bait to trap purified p38α in the presence of vehicle (DMSO), compound 50, or compound 46 (represented in micromolar concentrations). The rightmost lane represents a 1:25 dilution of initial input protein used in each pull-down. (B) Proximity-based AlphaLISA assay detects significant p38α:compound 50:VHL ternary complex, but no p38α:compound 46:VHL ternary complex. His-p38α and GST-VBC were incubated in the presence of increasing concentrations of compound 50 and compound 46 and the extent of ternary complex formation was assessed by excitation with incident light with 1=680 nm and capture of the emission light at 1=615 nm. Error bars represent the SD from quadruplicate experiments. (C) Only exemplary compound 50 ubiquitinates FLAG-p38α in HeLa cells. HeLa cells cotransfected with HA-Ubiquitin (HA-Ub) and FLAG-p38α were subsequently treated with either vehicle (DMSO) or 500 nM compound 50 or 500 nM compound 46 for 1 hour. FLAGimmunoprecipitated lysates were separated by SDS-PAGE and assessed via western blots detecting HA (Ub). Smears represent ubiquitin-conjugated FLAG-p38α and numerical markers to the left of the western blot refer to kilodalton (kDa) masses. WCL refers to “whole cell lysate” input.

FIGS. 15A, 15B, and 15C. Differential p38δ:PROTAC:VHL ternary complex affinities result in distinct cellular ubiquitination outcomes. (A) Both exemplary compound 50 and 46 pull-down p38δ in vitro, however compound 46 maintains ternary complex at lower concentrations. As in FIG. 14A, GST-tagged VBC was used as a bait to trap recombinant p38δ in the presence of vehicle (DMSO) or the indicated concentrations of compound 50 or 46. The rightmost lane represents a 1:25 dilution of initial input protein used in each pull-down. (B) Exemplary compound 46 forms a more stable ternary complex with endogenous p38δ and VHL than exemplary compound 50. GST-VBC was immobilized on glutathione sepharose beads and incubated with MDA-MB-231 whole cell lysate (WCL) in the presence of vehicle (DMSO) or the indicated concentrations of compound 50 or 46. Beads were washed and “trapped” proteins (i.e. those that engage in a ternary complex) were eluted with SDS sample buffer and separated by SDS-PAGE. Samples were assessed by western blot and CUL2 and α-tubulin serve as positive and negative GST-VBC immunoprecipitation controls, respectively. (C) Only exemplary compound 46 ubiquitinates FLAG-p38δ in HeLa cells. As in FIG. 14C, HeLa cells were co-transfected with HA-Ub and FLAG-p38δ and were either treated with vehicle (DMSO) or 1 μM of compound 50 or 1 μM of compound 46 for 2 hours. FLAGimmunoprecipitated lysates were separated by SDS-PAGE and assessed via western blots detecting HA (Ub). Smears represent ubiquitin-conjugated FLAG-p38δ and numerical markers to the left of the western blot refer to kilodalton (kDa) masses. WCL refers to “whole cell lysate” input. See FIGS. 16A, 16B, 16C, 17A, and 17B for additional p38δ:PROTAC:VHL characterization.

FIGS. 16A, 16B, and 16C. SPR curves assessing binary and ternary interactions. (A) Surface plasmon resonance (SPR) was used to measure the affinity of interaction (K_{d}) between His-p38δ and compound (foretinib, compound 46, or compound 50) and (B) GST-VBC and PROTAC (compound 46 or 50). (C) SPR kinetic evaluation of p38δ:compound 46:VHL and p38δ:compound 50:VHL ternary complex interactions. Equimolar p38δ:PROTAC mixtures were injected onto immobilized GST-VBC and allowed to dissociate overtime. The p38δ:compound 46:VHL ternary affinity is greater than the p38δ:compound 50:VHL ternary affinity owed, in part, to the slower k_{off} of the former. See Table 12 for a summary of the binary and ternary affinity and kinetic measurements.

FIGS. 17A and 17B. CETSA reveals significant exemplary compound 50- and exemplary compound 46-induced p38δ thermal shifts. (A) Cellular thermal shift assay (CETSA) was conducted in MDA-MB-231 cells as a means to monitor cellular target engagement. Cell lysate was incubated with either vehicle (DMSO), exemplary compound 50 (100 μM), or exemplary compound 46 (100 μM) for 30 minutes prior to melting at the indicated temperatures. Assay was performed in duplicate and western blots of p38δ and α-tubulin (negative control) are shown. (B) Raw band intensities from (A) were measured, averaged, and reported (with SD) with increasing temperature (51-55° C.) from left-to-right. Top: p38δ CETSA analysis. Paired t-tests were performed between DMSO and compound 46 and DMSO and compound 50, revealing significant thermal shifts with exemplary compound 46 (****p<0.0001) and exemplary compound 50 (*p=0.0121). Bottom: α-tubulin CETSA analysis. Paired t-tests were performed between DMSO and compound 46 and DMSO and compound 50, revealing no significant thermal shifts with either compound. n.s.=not significant.

FIG. 18. Exemplary compound 46 induces polyubiquitination of p38δ. (A) HeLa cells were co-transfected with HA-Ub and FLAG p38δ and treated with vehicle (DMSO), 1 μM of compound 50, or 1 μM of compound 46 for 3 hours. Lysates were immunoprecipitated with tandem ubiquitin binding entity (TUBE1) agarose beads and assessed by western blots detecting FLAG (p38δ). TUBE1 binds tetra ubiquitin chains with high affinity, thereby enriching for polyubiquitinated proteins. In this assay, HA (Ub) serves as an immunoprecipitation control for poly-Ub TUBE1 enrichment.

FIGS. 19A and 19B. MD simulations reveal differences in p38δ:PROTAC:VHL PPI interfaces due to variation in linker length and orientation of VHL recruiting moiety. (A) The compound 46-recruited VHL interacts with p38δ in a different mode than the compound 50-recruited VHL. p383, VHL, and either exemplary compound 46 or 50 were docked and a 100-ns molecular dynamics (MD) simulation relaxed the ternary structure. The p38δ:compound 46:VHL ternary complex is colored in tan and the p38δ:compound 50:VHL ternary complex is colored in dark blue. The p38δ structures from both ternary complex MD simulations were aligned and the resulting divergent VHL structures can be visualized. (B) Exemplary compound 46 and 50 PROTACs deviate at the exit of the p38δ kinase pocket to interact with the p38δ:VHL PPI interface in distinct ways. Using the same p38δ alignment as in (A), the p38δ and VHL structures were hidden such that individual PROTACs could be visualized in stick representation. Compound 46 carbons are colored in tan and compound 50 carbons are colored in dark blue with oxygen atoms colored in red, nitrogen atoms in blue, sulfur atoms in yellow, and fluorine atoms in pale blue for both PROTACs. Exemplary compounds 46 or 50 (which are composed of the same foretinib PTM) show profound alignment within the p38δ kinase, but deviate greatly upon exiting the pocket. Linker length differences between compound 46 (10-atoms) and 50 (13-atoms) reveal the “limits” in their respective ability to dock along the p38δ:VHL PPI interface. In addition, the orientation of attachment of the VHL-recruiting ligands result in hydroxyproline (Hyp) moieties that differ by ˜147° between the two PROTAC structures.

FIG. 20. Western blot assay measuring the total concentration of c-MET and the concentration of phosphorylated c-MET in cells incubated with the specified compounds at 3 μM concentration.

FIG. 21. Western blot assay measuring the concentration of the phosphorylated c-MET for the specified compounds at concentration from 0.003 to 3 μM.

FIG. 22. Western blot assay measuring the total concentration of c-MET and the concentration of phosphorylated c-MET in cells incubated with the specified compounds 3 μM concentration.

### DETAILED DESCRIPTION

The following is a detailed description provided to aid those skilled in the art in practicing the present disclosure. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

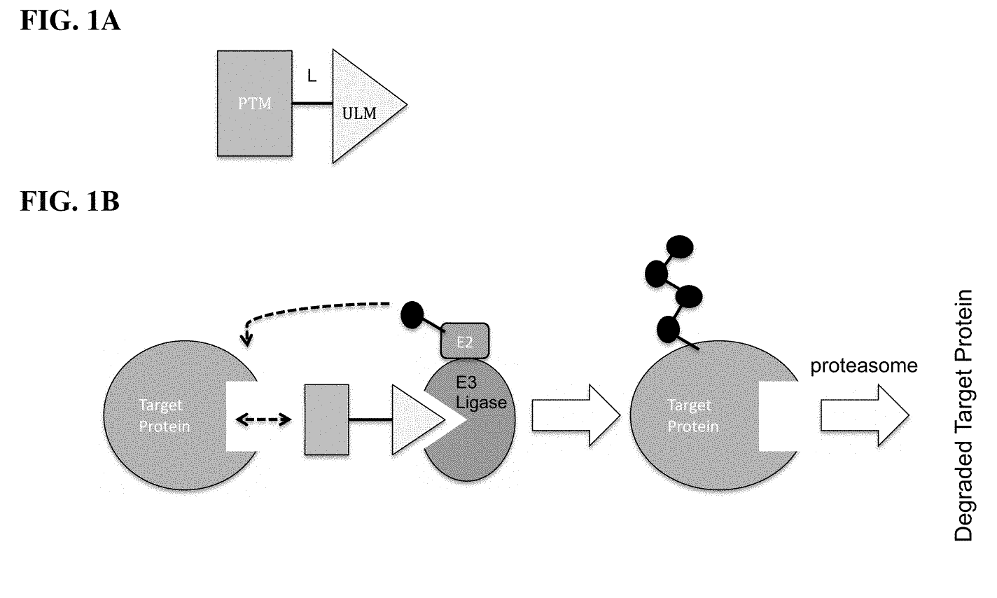

Presently described are compositions and methods that relate to the surprising and unexpected discovery that an E3 ubiquitin ligase protein (e.g., inhibitors of apoptosis proteins (IAP), a Von Hippel-Lindau E3 ubiquitin ligase (VHL), a cereblon E3 ubiquitin ligase, or a mouse double minute 2 homolog (MDM2) E3 ubiquitin ligase) ubiquitinates a target protein (e.g., cMet and/or p38) once it and the target protein are placed in proximity by a bifunctional or chimeric construct that binds the E3 ubiquitin ligase protein and the target protein. Accordingly the present disclosure provides such compounds and compositions comprising an E3 ubiquitin ligase binding moiety (“ULM”) coupled to a protein target binding moiety (“PTM”), which result in the ubiquitination of a chosen target protein (e.g., MET), which leads to the degradation of the target protein by the proteasome (see FIG. 1). The present disclosure also provides a library of compositions and the use thereof.

In certain aspects, the present disclosure provides compounds which comprise a ligand, e.g., a small molecule ligand (i.e., having a molecular weight of below 2,000, 1,000, 500, or 200 Daltons), which is capable of binding to a ubiquitin ligase, such as IAP, VHL, MDM2, or cereblon. The compounds also comprise a moiety that is capable of binding to target protein, in such a way that the target protein is placed in proximity to the ubiquitin ligase to effect degradation (and/or inhibition) of that protein. Small molecule can mean, in addition to the above, that the molecule is non-peptidyl, that is, it is not generally considered a peptide, e.g., comprises fewer than 4, 3, or 2 amino acids. In accordance with the present description, the PTM, ULM or PROTAC molecule can be a small molecule.

Unless otherwise defined, all technical and scientific terms used herein have the same meaning as commonly understood by one of ordinary skill in the art to which this disclosure belongs. The terminology used in the description is for describing particular embodiments only and is not intended to be limiting of the present disclosure.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

The following terms are used to describe the present disclosure. In instances where a term is not specifically defined herein, that term is given an art-recognized meaning by those of ordinary skill applying that term in context to its use in describing the present invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are coadministered in combination with at least one additional bioactive agent, especially including an anticancer agent or anti-inflammatory agent. In particularly preferred aspects, the co-administration of compounds results in synergistic activity and/or therapy, including anticancer activity or anti-inflammatory activity.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives, including prodrug and/or deuterated forms thereof where applicable, in context. Deuterated small molecules contemplated are those in which one or more of the hydrogen atoms contained in the drug molecule have been replaced by deuterium.

Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder. When the bond is shown, both a double bond and single bond are represented or understood within the context of the compound shown and well-known rules for valence interactions.

The term “ubiquitin ligase” refers to a family of proteins that facilitate the transfer of ubiquitin to a specific substrate protein, targeting the substrate protein for degradation. For example, TAP an E3 ubiquitin ligase protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead be altered in their cellular location or function, for example, via binding other proteins that have domains capable of binding ubiquitin. Further complicating matters, different lysines on ubiquitin can be targeted by an E3 to make chains. The most common lysine is Lys48 on the ubiquitin chain. This is the lysine used to make polyubiquitin, which is recognized by the proteasome.

The term “patient” or “subject” is used throughout the specification to describe an animal, preferably a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The term “effective” is used to describe an amount of a compound, composition or component which, when used within the context of its intended use, effects an intended result. The term effective subsumes all other effective amount or effective concentration terms, which are otherwise described or used in the present application.

The term “p38” is used throughout the present disclosure to refer to each member of the p38 MAPK family members individually, including p38α, p38β, p38γ, and/or p38δ, as well as the p38 MAPK family as a group.

Compounds and Compositions

In one aspect, the description provides compounds comprising an E3 ubiquitin ligase binding moiety (“ULM”) that is an IAP E3 ubiquitin ligase binding moiety (an “ILM”), a cereblon E3 ubiquitin ligase binding moiety (a “CLM”), a Von Hippel-Lindae E3 ubiquitin ligase (VHL) binding moiety (VLM), and/or a mouse double minute 2 homologue (MDM2) E3 ubiquitin ligase binding moiety (MLM). In an exemplary embodiment, the ULM is coupled to a target protein binding moiety (PTM) via a chemical linker (L) according to the structure:

PTM-L-ULM (A)

wherein L is a bond or a chemical linker group, ULM is a E3 ubiquitin ligase binding moiety, and PTM is a target protein binding moiety. The number and/or relative positions of the moieties in the compounds illustrated herein are provided by way of example only. As would be understood by the skilled artisan, compounds described herein can be synthesized with any desired number and/or relative position of the respective functional moieties.

The terms ULM, ILM, VLM, MLM, and CLM are used in their inclusive sense unless the context indicates otherwise. For example, the term ULM is inclusive of all ULMs, including those that bind IAP (i.e., ILMs), MDM2 (i.e., MLM), cereblon (i.e., CLM), and VHL (i.e., VLM). Further, the term ILM is inclusive of all possible IAP E3 ubiquitin ligase binding moieties, the term MLM is inclusive of all possible MDM2 E3 ubiquitin ligase binding moieties, the term VLM is inclusive of all possible VHL binding moieties, and the term CLM is inclusive of all cereblon binding moieties.

In another aspect, the present disclosure provides bifunctional or multifunctional compounds (e.g., PROTACs) useful for regulating protein activity by inducing the degradation of a target protein. In certain embodiments, the compound comprises an ILM or a VLM or a CLM or a MLM coupled, e.g., linked covalently, directly or indirectly, to a moiety that binds a target protein (i.e., a protein targeting moiety or a “PTM”). In certain embodiments, the ILM/VLM/CLM/MLM and PTM are joined or coupled via a chemical linker (L). The ILM binds the IAP E3 ubiquitin ligase, the VLM binds VHL, CLM binds the cereblon E3 ubiquitin ligase, and MLM binds the MDM2 E3 ubiquitin ligase, and the PTM recognizes a target protein and the interaction of the respective moieties with their targets facilitates the degradation of the target protein by placing the target protein in proximity to the ubiquitin ligase protein. An exemplary bifunctional compound can be depicted as:

PTM-ILM (B)

PTM-CLM (C)

PTM-VLM (D)

PTM-MLM (E)

In certain embodiments, the bifunctional compound further comprises a chemical linker (“L”). For example, the bifunctional compound can be depicted as:

PTM-L-ILM (F)

PTM-L-CLM (G)

PTM-L-VLM (H)

PTM-L-MLM (I)

wherein the PTM is a protein/polypeptide targeting moiety, the L is a chemical linker, the ILM is a IAP E3 ubiquitin ligase binding moiety, the CLM is a cereblon E3 ubiquitin ligase binding moiety, the VLM is a VHL binding moiety, and the MLM is a MDM2 E3 ubiquitin ligase binding moiety.

In certain embodiments, the ULM (e.g., a ILM, a CLM, a VLM, or a MLM) shows activity or binds to the E3 ubiquitin ligase (e.g., IAP E3 ubiquitin ligase, cereblon E3 ubiquitin ligase, VHL, or MDM2 E3 ubiquitin ligase) with an IC₅₀ of less than about 200 μM. The IC₅₀ can be determined according to any method known in the art, e.g., a fluorescent polarization assay.

In certain additional embodiments, the bifunctional compounds described herein demonstrate an activity with an IC₅₀ of less than about 100, 50, 10, 1, 0.5, 0.1, 0.05, 0.01, 0.005, 0.001 mM, or less than about 100, 50, 10, 1, 0.5, 0.1, 0.05, 0.01, 0.005, 0.001 μM, or less than about 100, 50, 10, 1, 0.5, 0.1, 0.05, 0.01, 0.005, 0.001 nM, or less than about 100, 50, 10, 1, 0.5, 0.1, 0.05, 0.01, 0.005, 0.001 pM.

In certain embodiments, the compounds as described herein comprise multiple PTMs (targeting the same or different protein targets), multiple ULMs, one or more ULMs (i.e., moieties that bind specifically to multiple/different E3 ubiquitin ligase, e.g., VHL, IAP, cereblon, and/or MDM2) or a combination thereof. In any of the aspects or embodiments described herein, the PTMs and ULMs (e.g., ILM, VLM, CLM, and/or MLM) can be coupled directly or via one or more chemical linkers or a combination thereof. In additional embodiments, where a compound has multiple ULMs, the ULMs can be for the same E3 ubiquitin ligase or each respective ULM can bind specifically to a different E3 ubiquitin ligase. In still further embodiments, where a compound has multiple PTMs, the PTMs can bind the same target protein (e.g., the multiple PTMs target cMet and/or p38) or each respective PTM can bind specifically to a different target protein (e.g., one PTM targets cMet and/or p38, and while other PTM(s) target a different target protein or proteins).

In certain embodiments, where the compound comprises multiple ULMs, the ULMs are identical. In additional embodiments, the compound comprising a plurality of ULMs (e.g., ULM, ULM′, etc.), at least one PTM coupled to a ULM directly or via a chemical linker (L) or both. In certain additional embodiments, the compound comprising a plurality of ULMs further comprises multiple PTMs. In still additional embodiments, the PTMs are the same or, optionally, different. In still further embodiments, wherein the PTMs are different, the respective PTMs may bind the same protein target or bind specifically to a different protein target.

In certain embodiments, the compound may comprise a plurality of ULMs and/or a plurality of ULM's. In further embodiments, the compound comprising at least two different ULMs, a plurality of ULMs, and/or a plurality of ULM's further comprises at least one PTM coupled to a ULM or a ULM′ directly or via a chemical linker or both. In any of the embodiments described herein, a compound comprising at least two different ULMs can further comprise multiple PTMs. In still additional embodiments, the PTMs are the same or, optionally, different. In still further embodiments, wherein the PTMs are different, the respective PTMs may bind the same protein target or bind specifically to a different protein target. In still further embodiments, the PTM itself is a ULM (or ULM′), such as an ILM, a VLM, a CLM, a MLM, an ILM′, a VLM′, a CLM′, and/or a MLM′.

In additional embodiments, the description provides the compounds as described herein including their enantiomers, diastereomers, solvates and polymorphs, including pharmaceutically acceptable salt forms thereof, e.g., acid and base salt forms.

Exemplary ILMs

AVPI Tetrapeptide Fragments

In any of the compounds described herein, the ILM can comprise an alanine-valine-proline-isoleucine (AVPI) tetrapeptide fragment or an unnatural mimetic thereof. In certain embodiments, the ILM is selected from the group consisting of chemical structures represented by Formulas (I), (II), (III), (IV), and (V):

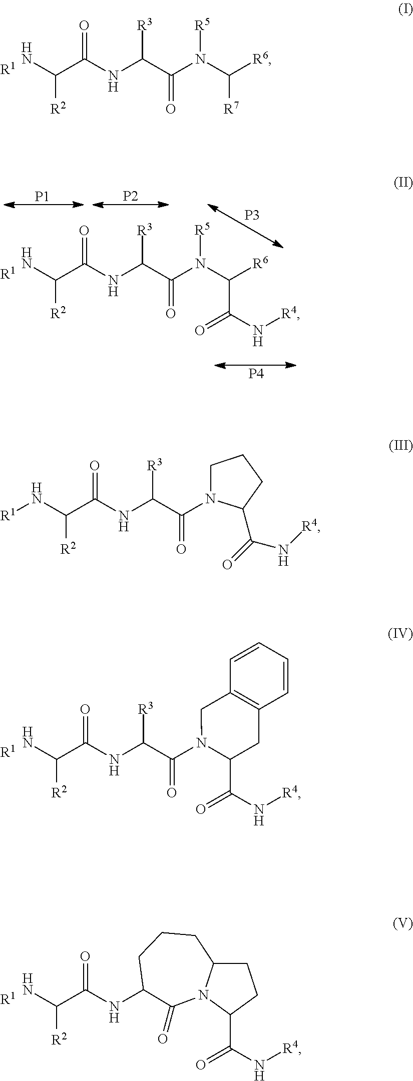

wherein:
R¹ for Formulas (I), (II), (III), (IV), and (V) is selected from H or alkyl;
R² for Formulas (I), (II), (III), (IV), and (V) is selected from H or alkyl;
R³ for Formulas (I), (II), (III), (IV), and (V) is selected from H, alkyl, cycloalkyl and heterocycloalkyl;
R⁵ and R⁶ for Formulas (I), (II), (III), (IV), and (V) are independently selected from H, alkyl, cycloalkyl, heterocycloalkyl, or more preferably, R⁵ and R⁶ taken together for Formulas (I), (II), (III), (IV), and (V) form a pyrrolidine or a piperidine ring further optionally fused to 1-2 cycloalkyl, heterocycloalkyl, aryl or heteroaryl rings, each of which can then be further fused to another cycloalkyl, heterocycloalkyl, aryl or heteroaryl ring;
R³ and R⁵ for Formulas (I), (II), (III), (IV), and (V) taken together can form a 5-8-membered ring further optionally fused to 1-2 cycloalkyl, heterocycloalkyl, aryl or heteroaryl rings;
R⁷ for Formulas (I), (II), (III), (IV), and (V) is selected from cycloalkyl, cycloalkylalkyl, heterocycloalkyl, heterocycloalkylalkyl, aryl, arylalkyl, heteroaryl or heteroarylalkyl, each one further optionally substituted with 1-3 substituents selected from halogen, alkyl, haloalkyl, hydroxyl, alkoxy, cyano, (hetero)cycloalkyl or (hetero)aryl, or R⁷ is —C(O)NH—R⁴; and
R⁴ is selected from alkyl, cycloalkyl, heterocycloalkyl, cycloalkylalkyl, heterocycloalkylalkyl, aryl, arylalkyl, heteroaryl, heteroarylalkyl, further optionally substituted with 1-3 substituents as described above.

As shown above, P1, P2, P3, and P4 of Formular (II) correlate with A, V, P, and I, respectively, of the AVPI tetrapeptide fragment or an unnatural mimetic thereof. Similarly, each of Formulas (I) and (III) through (V) have portions correlating with A, V, P, and I of the AVPI tetrapeptide fragment or an unnatural mimetic thereof.

In any of the compounds described herein, the ILM can have the structure of Formula (VI), which is a derivative of IAP antagonists described in WO Pub. No. 2008/014236, or an unnatural mimetic thereof:

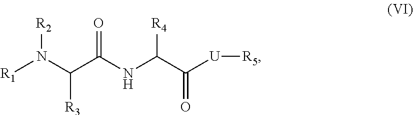

wherein:
R₁ of Formula (VI) is, independently selected from H, C₁-C₄-alky, C₁-C₄-alkynyl or C₃-C₁₀-cycloalkyl which are unsubstituted or substituted; R₂ of Formula (VI) is, independently selected from H, C₁-C₄-alkyl, C₁-C₄-alkynyl or C₃-C₁₀-cycloalkyl which are unsubstituted or substituted;
R₃ of Formula (VI) is, independently selected from H, —CF₃, —C₂H₅, C₁-C₄-alkyl, C₁-C₄-alkenyl, C₁-C₄ alkenyl, —CH₂—Z or any R₂ and R₃ together form a heterocyclic ring;
each Z of Formula (VI) is, independently selected from H, —OH, F, Cl, —CH₃, —CF₃, —CH₂Cl, —CH₂F or —CH₂OH;
R₄ of Formula (VI) is, independently selected from C₁-C₁₆ straight or branched alkyl, C₁-C₁₆-alkenyl, C₃-C₁₆-alkynyl, C₃-C₁₀-cycloalkyl, —(CH₂)₀₋₆—Z₁, —(CH₂)₀₋₆-aryl, and —(CH₂)₀₋₆-het, wherein alkyl, cycloalkyl, and phenyl are unsubstituted or substituted;
R₅ of Formula (VI) is, independently selected from H, C₁₋₁₀-alkyl, aryl, phenyl, C₃₋₇-cycloalkyl, —(CH₂)₁₋₆—C₃₋₇— cycloalkyl, C₁₋₁₀-allyl aryl, —(CH₂)₀₋₆—C₃₋₇-cycloalkyl-(CH₂)₀₋₆-phenyl, —(CH₂)₀₋₄—CH[(CH₂)₁₋₄— phenyl]₂, indanyl, —C(O)₁₋₁₀-alkyl, C(O)—(CH₂)₁₋₆—C₃₋₇-cycloalkyl, —C(O)—(CH₂)₀₋₆-phenyl, —(CH₂)₀₋₆—C(O)-phenyl, —(CH₂)₀₋₆-het, —C(O)—(CH₂)₁₋₆-het, or R₅ is selected from a residue of an amino acid, wherein the alkyl, cycloalkyl, phenyl, and aryl substituents are unsubstituted or substituted;
Z₁ of Formula (VI) is, independently selected from —N(R₁₀)—C(O)—C₁₋₁₀-alkyl, —N(R₁₀)—C(O)—(CH₂)₀₋₆—C₃₋₇-cycloalkyl, —N(R₁₀)—C(O)—(CH₂)₀₋₆-phenyl, —N(R₁₀)—C(O)(CH₂)₁₋₆-het, —C(O)—N(R₁₁)(R₁₂), —C(O)—O—C₁₋₁₀-alkyl, —C(O)—O—(CH₂)₁₋₆—C₃₋₇-cycloalkyl, —C(O)—O—(CH₂)₀₋₆-phenyl, —C(O)—O—(CH₂)₁₋₆-het, —O—C(O)—C₁₋₁₀-alkyl, —O—C(O)—(CH₂)₁₋₆—C₃₋₇-cycloalkyl, —O—C(O)—(CH₂)₀₋₆-phenyl, —O—C(O)—(CH₂)₁₋₆-het, wherein alkyl, cycloalkyl, and phenyl are unsubstituted or substituted;
het of Formula (VI) is, independently selected from a 5-7 member heterocyclic ring containing 1-4 heteroatoms selected from N, O, and S, or an 8-12 member fused ring system including at least one 5-7 member heterocyclic ring containing 1, 2, or 3 heteroatoms selected from N, O, and S, which heterocyclic ring or fused ring system is unsubstituted or substituted on a carbon or nitrogen atom;
R₁₀ of Formula (VI) is selected from H, —CH₃, —CF₃, —CH₂OH, or —CH₂Cl;
R₁₁ and R₁₂ of Formula (VI) are independently selected from H, C₁₋₄-alkyl, C₃₋₇-cycloalkyl, —(CH₂)₁₋₆—C₃₋₇— cycloakyl, (CH₂)₀₋₆-phenyl, wherein alkyl, cycloalkyl, and phenyl are unsubstituted or substituted; or R₁₁ and R₁₂ together with the nitrogen form het, and
U of Formula (VI) is, independently, as shown in Formula (VII):

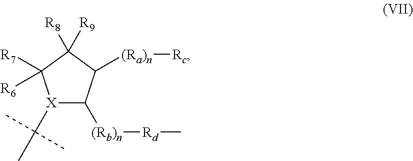

wherein:
each n of Formula (VII) is, independently selected from 0 to 5;
X of Formula (VII) is selected from the group —CH and N;
Rₐ and R_{b}, of Formula (VII) are independently selected from the group O, S, or N atom or C₀₋₈-alkyl wherein one or more of the carbon atoms in the alkyl chain are optionally replaced by a heteroatom selected from O, S, or N, and where each alkyl is, independently, either unsubstituted or substituted;
R_{d} of Formula (VII) is selected from the group Re-Q-(R_{f})ₚ(R_{g})_{q}, and Ar₁-D-Ar₂;
R_{c} of Formula (VII) is selected from the group H or any R_{c} and R_{d} together form a cycloalkyl or het; where if R_{c} and R_{d} form a cycloalkyl or het, R₅ is attached to the formed ring at a C or N atom;
p and q of Formula (VII) are independently selected from 0 or 1;
Rₑ of Formula (VII) is selected from the group C₁₋₈-alkyl and alkylidene, and each Re is either unsubstituted or substituted;
Q is selected from the group N, O, S, S(O), and S(O)₂;
Ar₁ and Ar₂ of Formula (VII) are independently selected from the group of substituted or unsubstituted aryl and het;
R_{f} and R_{g} of Formula (VII) are independently selected from H, —C1-10-alkyl, C₁₋₁₀-alkylaryl, —OH, —O—C₁₋₁₀-alkyl, —(CH₂)₀₋₆—C₃₋₇-cycloalky, —O—(CH₂)₀₋₆-aryl, phenyl, aryl, phenyl-phenyl, —(CH₂)₁₋₆-het, —O—(CH₂)₁₋₆-het, —OR₁₃, —C(O)—R₁₃, —C(O)—N(R₁₃)(R₁₄), —N(R₁₃)(R₁₄), —S—R₁₃—S(O)—R₁₃, —S(O)₂—R₁₃, —S(O)₂—NR₁₃R₁₄, —NR₁₃—S(O)₂—R₁₄, —S—C₁₋₁₀-aryl-C₁₋₄-alkyl, or het-C₁₋₄-alkyl, wherein alkyl, cycloalkyl, het, and aryl are unsubstituted or substituted, —SO₂—C₁₋₂-alkyl, —SO₂—C₁₋₂-alkylphenyl, —O—C₁₋₄-alkyl, or any R_{g} and R_{f} together form a ring selected from het or aryl;
D of Formula (VII) is selected from the group —CO—, —C(O)—C₁₋₇-alkylene or arylene, —CF₂—, —O—, —S(O)ᵣ where r is 0-2, 1,3-dioxalane, or C₁₋₇-alkyl-OH; where alkyl, alkylene, or arylene are unsubstituted or substituted with one or more halogens, OH, —O—C₁₋₆-alkyl, —S—C₁₋₆-alkyl, or —CF₃; or each D is, independently selected from N(Rₕ);
Rh is selected from the group H, unsubstituted or substituted C₁₋₇-alkyl, aryl, unsubstituted or substituted —O—(C₁₋₇-cycloalkyl), —C(O)—C₁₋₁₀-alkyl, —C(O)—C₀₋₁₀-alkyl aryl, —C—O—C₀₁₋₁₀-alkyl, —C—O—C₀₋₁₀-alkyl-aryl, —SO₂—C₁₋₁₀-alkyl, or —SO₂—(C₀₋₁₀— alkylaryl);
R₆, R₇, R₈, and R₉ of Formula (VII) are, independently, selected from the group H, —C₁₋₁₀-alkyl, —C₁₋₁₀-alkoxy, aryl C₁₋₁₀-alkoxy, —OH, —O—C₁₋₁₀-alkyl, —(CH₂)₀₋₆—C₃₋₇-cycloalkyl, —O—(CH₂)₀₋₆-aryl, phenyl, —(CH₂)₁₋₆-het, —O—(CH₂)₁₋₆-het, —OR₁₃, —C(O)—R₁₃, —C(O)—N(R₁₃)(R₁₄), —N(R₁₃)(R₁₄), —S—R₁₃, —S(O)—R₁₃, —S(O)₂—R₁₃, —S(O)₂—NR₁₃R₁₄, or —NR₁₃—S(O)₂—R₁₄; wherein each alkyl, cycloalkyl, and aryl is unsubstituted or substituted; and any R₆, R₇, R₈, and R₉ optionally together form a ring system;
R₁₃ and R₁₄ of Formula (VII) are independently selected from the group H, —C₁₋₁₀-alkyl, —(CH₂)₀₋₆—C₃₋₇-cycloalkyl, —(CH₂)₀₋₆— (CH)₀₋₁-(aryl)₁₋₂, —C(O)—C₁₋₁₀-alkyl, —C(O)—(CH₂)₁₋₆—C₃₋₇-cycloalkyl, —C(O)—O—(CH₂)₀₋₆-aryl, —C(O)—(CH₂)₀₋₆—O-fluorenyl, —C(O)—NH—(CH₂)₀₋₆-aryl, —C(O)—(CH₂)₀₋₆-aryl, —C(O)—(CH₂)₀₋₆-het, —C(S)—C₁₋₁₀-alkyl, —C(S)—(CH₂)₁₋₆—C₃₋₇-cycloalkyl, —C(S)—O—(CH₂)₀₋₆-aryl, —C(S)—(CH₂)₀₋₆—O-fluorenyl, —C(S)—NH—(CH₂)₀₋₆-aryl, —C(S)—(CH₂)₀₋₆-aryl, or —C(S)—(CH₂)₁₋₆-het, wherein each alkyl, cycloalkyl, and aryl is unsubstituted or substituted: or any R₁₃ and R₁₄ together with a nitrogen atom form het;
wherein alkyl substituents of R₁₃ and R₁₄ of Formula (VII) are unsubstituted or substituted and when substituted, are substituted by one or more substituents selected from C₁₋₁₀-alkyl, halogen, OH, —O—C₁₋₆-alkyl, —S—C₁₋₆-alkyl, and —CF₃; and substituted phenyl or aryl of R₁₃ and R₁₄ are substituted by one or more substituents selected from halogen, hydroxyl. C₁₋₄-alkyl, C₁₋₄-alkoxy, nitro, —CN, —O—C(O)—C₁₋₄-alkyl, and —C(O)—O—C₁₋₄-aryl; or a pharmaceutically, acceptable salt or hydrate thereof.

In certain embodiments, the compound further comprises an independently selected second ILM attached to the ILM of Formula (VI), or an unnatural mimetic thereof, by way of at least one additional independently selected linker group. In an embodiment, the second ILM is a derivative of Formula (VI), or an unnatural mimetic thereof. In a certain embodiment, the at least one additional independently selected linker group comprises two additional independently selected linker groups chemically linking the ILM and the second ILM. In an embodiment, the at least one additional linker group for an ILM of the Formula (VI), or an unnatural mimetic thereof, chemically links groups selected from R₄ and R₅. For example, an ILM of Formula (VI) and a second ILM of Formula (VI), or an unnatural mimetic thereof, can be linked as shown below:

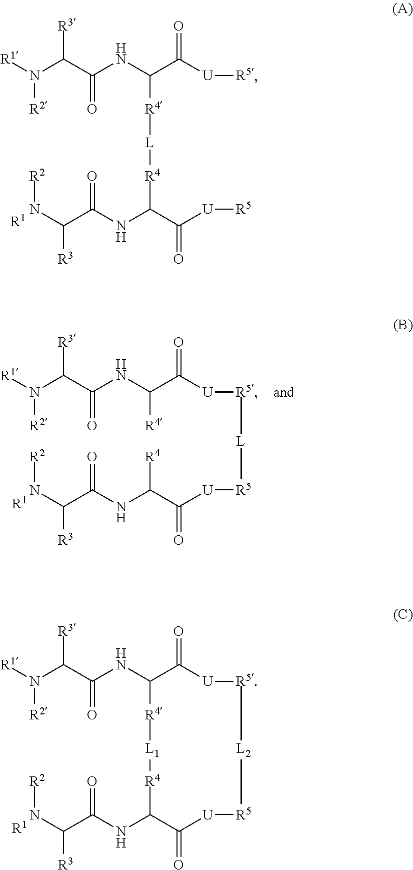

In certain embodiments, the ILM, the at least one additional independently selected linker group L, and the second ILM has a structure selected from the group consisting of:

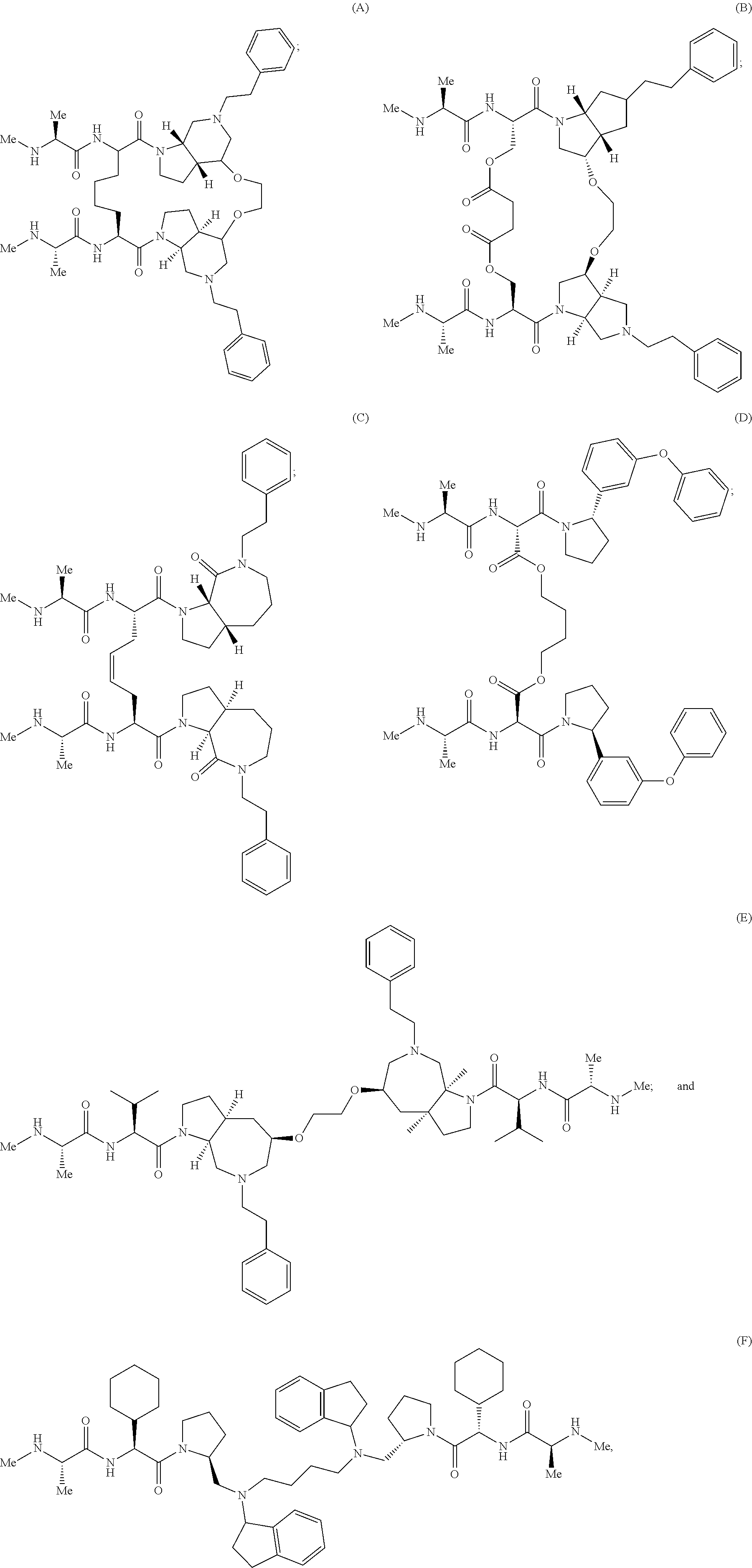

which are derivatives of IAP antagonists described in WO Publication No. 2008/014236.

In any of the compounds described herein, the ILM can have the structure of Formula (VIII), which is based on the IAP ligands described in Ndubaku, C., et al. Antagonism of c-IAP and XIAP proteins is required for efficient induction of cell death by small-molecule IAP antagonists, ACS Chem. Biol., 557-566, 4 (7) (2009), or an unnatural mimetic thereof:

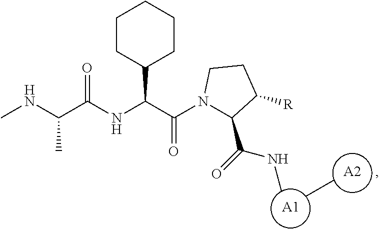

wherein each of A1 and A2 of Formula (VIII) is independently selected from optionally substituted monocyclic, fused rings, aryls and hetoroaryls; and

R of Formula (VIII) is selected from H or Me.

In a particular embodiment, the linker group L is attached to A1 of Formula (VIII). In another embodiment, the linker group L is attached to A2 of Formula (VIII).

In a particular embodiment, the ILM is selected from the group consisting of

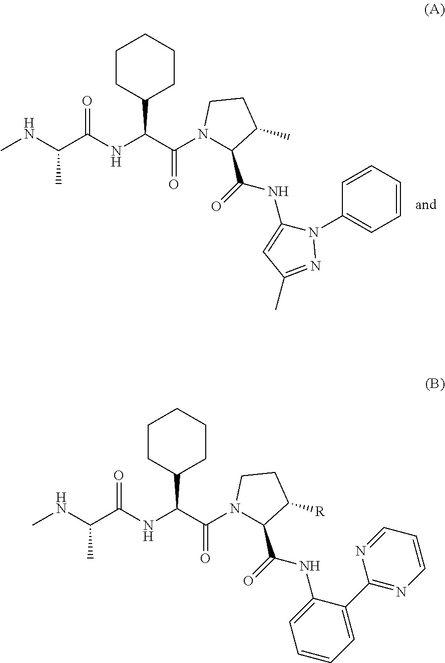

In any of the compounds described herein, the ILM can have the structure of Formula (IX), which is derived from the chemotypes cross-referenced in Mannhold, R., et al. IAP antagonists: promising candidates for cancer therapy, Drug Discov. Today, 15 (5-6), 210-9 (2010), or an unnatural mimetic thereof:

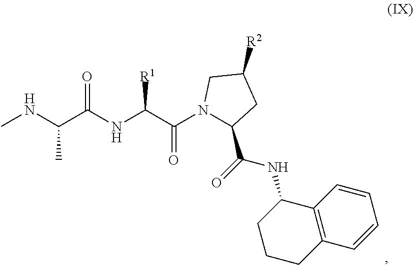

wherein R¹ is selected from alkyl, cycloalkyl and heterocycloalkyl and, most preferably, from isopropyl, tert-butyl, cyclohexyl and tetrahydropyranyl, and R² of Formula (IX) is selected from —OPh or H.

In any of the compounds described herein, the ILM can have the structure of Formula (X), which is derived from the chemotypes cross-referenced in Mannhold, R., et al. IAP antagonists: promising candidates for cancer therapy, Drug Discov. Today, 15 (5-6), 210-9 (2010), or an unnatural mimetic thereof:

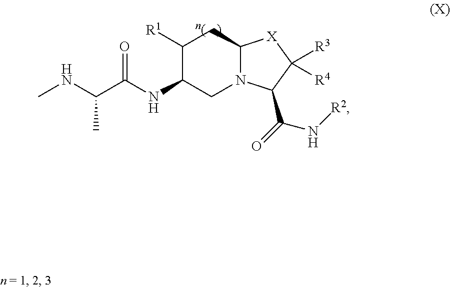

wherein:

R¹ of Formula (X) is selected from H, —CH₂OH, —CH₂CH₂OH, —CH₂NH₂, —CH₂CH₂NH₂;

X of Formula (X) is selected from S or CH₂;

R² of Formula (X) is selected from:

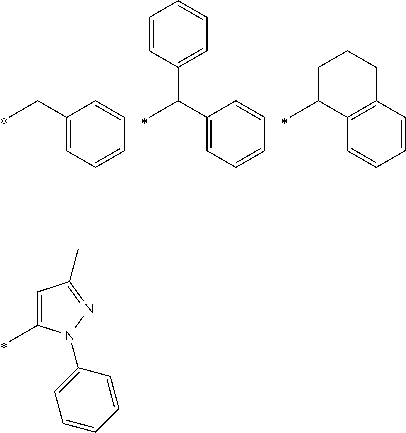

R³ and R⁴ of Formula (X) are independently selected from H or Me

In any of the compounds described herein, the ILM can have the structure of Formula (XI), which is derived from the chemotypes cross-referenced in Mannhold, R., et al. IAP antagonists: promising candidates for cancer therapy, Drug Discov. Today, 15 (5-6), 210-9 (2010), or an unnatural mimetic thereof:

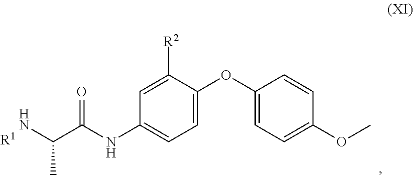

wherein R¹ of Formula (XI) is selected from H or Me, and R² of Formula (XI) is selected from H or

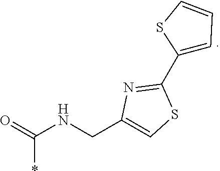 [SMILES: *C(=O)NCc1csc(-c2cccs2)n1]

In any of the compounds described herein, the ILM can have the structure of Formula (XII), which is derived from the chemotypes cross-referenced in Mannhold, R., et al. IAP antagonists: promising candidates for cancer therapy, Drug Discov. Today, 15 (5-6), 210-9 (2010), or an unnatural mimetic thereof:

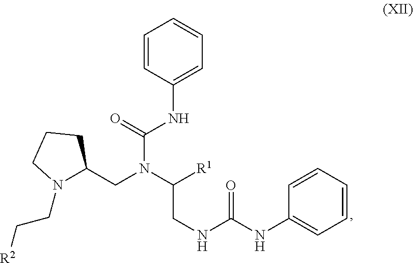

wherein:

R¹ of Formula (XII) is selected from:

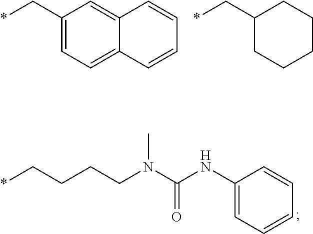

and

R² of Formula (XII) is selected from:

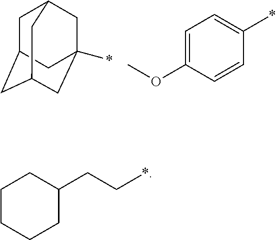

In any of the compounds described herein, the IAP E3 ubiquitin ligase binding moiety is selected from the group consisting of:

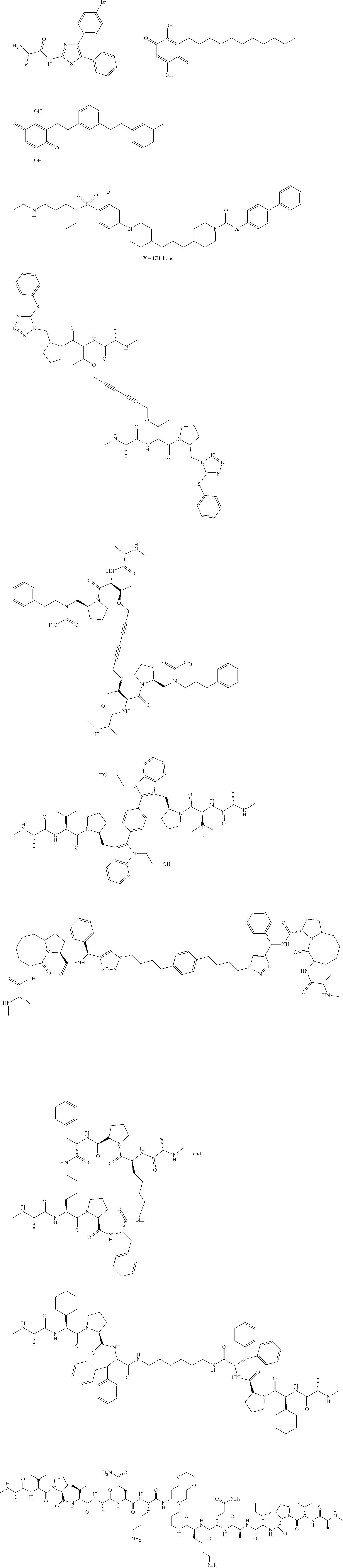

In any of the compounds described herein, the ILM can have the structure of Formula (XIII), which is based on the IAP ligands summarized in Flygare, J. A., et al. Small-molecule pan-IAP antagonists: a patent review, Expert Opin. Ther. Pat., 20 (2), 251-67 (2010), or an unnatural mimetic thereof:

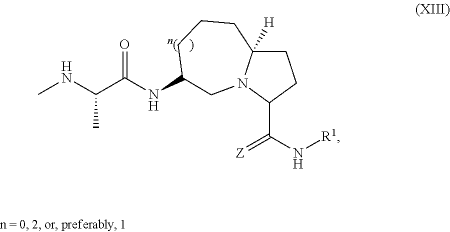

wherein:

Z of Formula (XIII) is absent or O;

R¹ of Formula (XIII) is selected from:

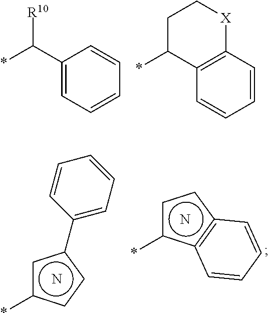

R¹⁰ of

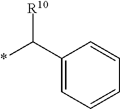

is selected from H, alkyl, or aryl;

X is selected from CH2 and O; and

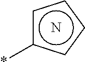

is a nitrogen-containing heteroaryl.

In any of the compounds described herein, the ILM can have the structure of Formula (XIV), which is based on the IAP ligands summarized in Flygare, J. A., et al. Small-molecule pan-IAP antagonists: a patent review, Expert Opin. Ther. Pat., 20 (2), 251-67 (2010), or an unnatural mimetic thereof:

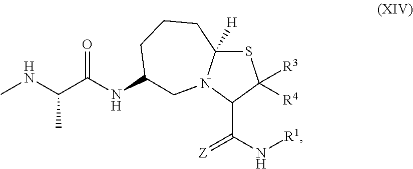

wherein:

Z of Formula (XIV) is absent or O;

R³ and R⁴ of Formula (XIV) are independently selected from H or Me;

R¹ of Formula (XIV) is selected from:

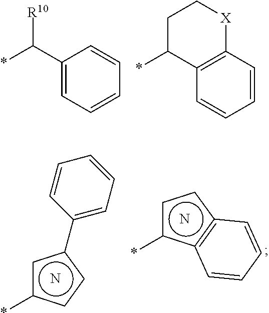

R¹⁰ of

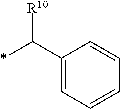

is selected from H, alkyl, or aryl;

X of

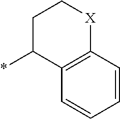

is selected from CH2 and O; and

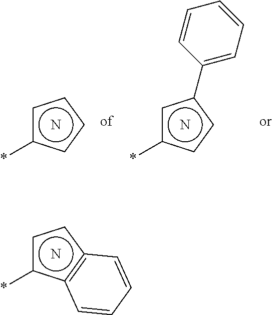

is a nitrogen-containing heteraryl.

In any of the compounds described herein, the ILM is selected from the group consisting of:

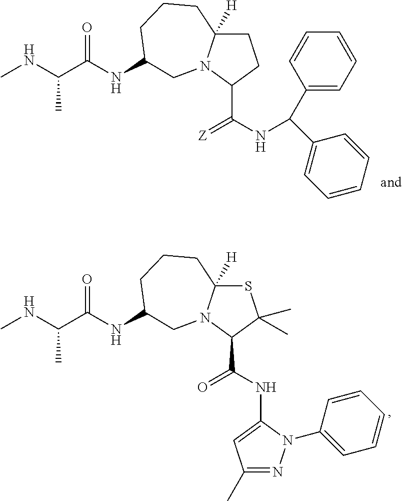

which are derivatives of ligands disclose in US Patent Pub. No. 2008/0269140 and U.S. Pat. No. 7,244,851.

In any of the compounds described herein, the ILM can have the structure of Formula (XV), which was a derivative of the IAP ligand described in WO Pub. No. 2008/128171, or an unnatural mimetic thereof:

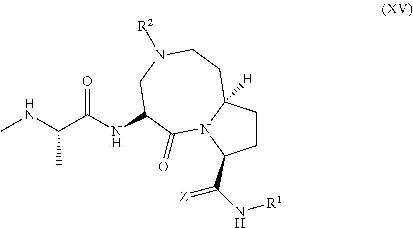

wherein:

Z of Formula (XV) is absent or O;

R¹ of Formula (XV) is selected from:

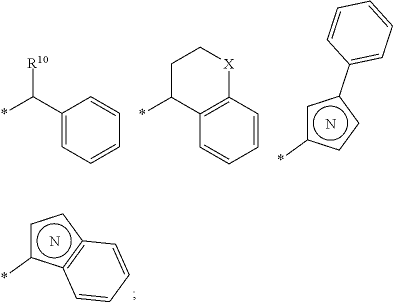

R¹⁰ of

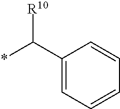

is selected from H, alkyl, or aryl;

X of

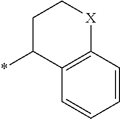

is selected from CH2 and O; and

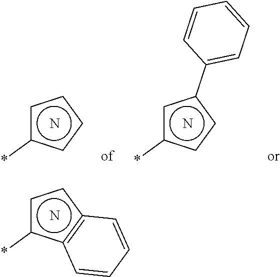

is a nitrogen-containing heteraryl; and
R² of Formula (XV) selected from H, alkyl, or acyl;

In a particular embodiment, the ILM has the following structure:

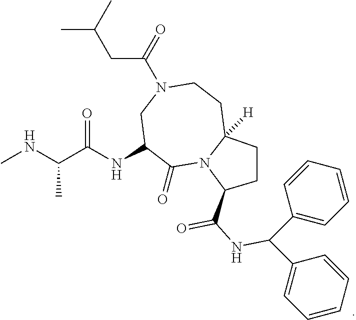

In any of the compounds described herein, the ILM can have the structure of Formula (XVI), which is based on the IAP ligand described in WO Pub. No. 2006/069063, or an unnatural mimetic thereof:

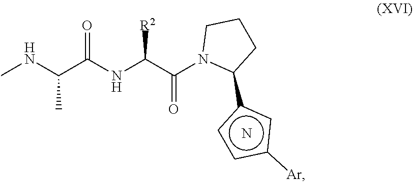

wherein:
R² of Formula (XVI) is selected from alkyl, cycloalkyl and heterocycloalkyl; more preferably, from isopropyl, tert-butyl, cyclohexyl and tetrahydropyranyl, most preferably from cyclohexyl;

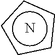

of Formula (XVI) is a 5- or 6-membered nitrogen-containing heteroaryl; more preferably, 5-membered nitrogen-containing heteroaryl, and most preferably thiazole; and Ar of Formula (XVI) is an aryl or a heteroaryl.

In any of the compounds described herein, the ILM can have the structure of Formula (XVII), which is based on the IAP ligands described in Cohen, F. et al., Antogonists of inhibitors of apoptosis proteins based on thiazole amide isosteres, Bioorg. Med. Chem. Lett., 20(7), 2229-33 (2010), or an unnatural mimetic thereof:

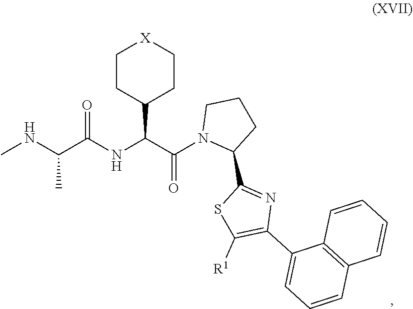

wherein:

R¹ of Formula (XVII) is selected from to group halogen (e.g. fluorine), cyano,

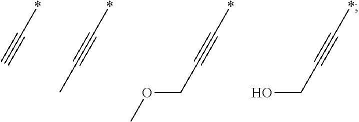

X of Formula (XVII) is selected from the group O or CH2.

In any of the compounds described herein, the ILM can have the structure of Formula (XVIII), which is based on the IAP ligands described in Cohen, F. et al., Antogonists of inhibitors of apoptosis proteins based on thiazole amide isosteres, Bioorg. Med. Chem. Lett., 20(7), 2229-33 (2010), or an unnatural mimetic thereof:

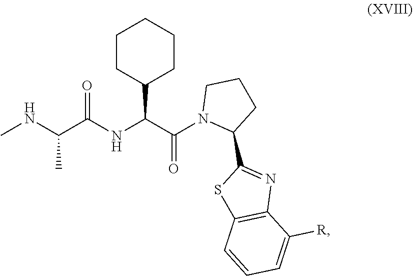

wherein R of Formula (XVIII) is selected from alkyl, aryl, heteroaryl, arylalkyl, heteroarylalkyl or halogen (in variable substitution position).

In any of the compounds described herein, the ILM can have the structure of Formula (XIX), which is based on the IAP ligands described in Cohen, F. et al., Antogonists of inhibitors of apoptosis proteins based on thiazole amide isosteres, Bioorg. Med. Chem. Lett., 20(7), 2229-33 (2010), or an unnatural mimetic thereof:

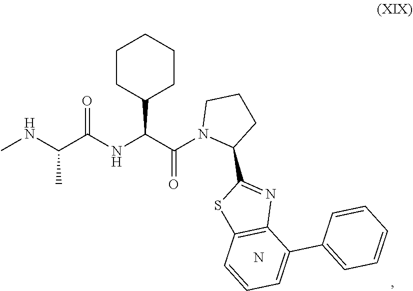

wherein

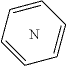

is a 6-member nitrogen heteroaryl.

In a certain embodiment, the ILM of the composition is selected from the group consisting of:

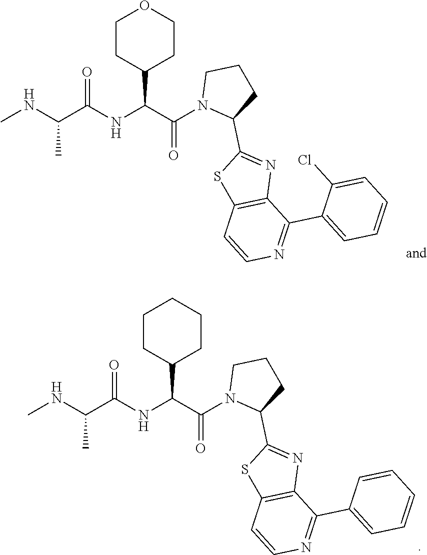 [SMILES: CN[C@@H](C)C(=O)N[C@H](C(=O)N1CCC[C@H]1c1nc2c(-c3ccccc3Cl)nccc2s1)C1CCOCC1 ; CN[C@@H](C)C(=O)N[C@H](C(=O)N1CCC[C@H]1c1nc2c(-c3ccccc3)nccc2s1)C1CCCCC1]

In certain embodiments, the ILM of the composition is selected from the group consisting of:

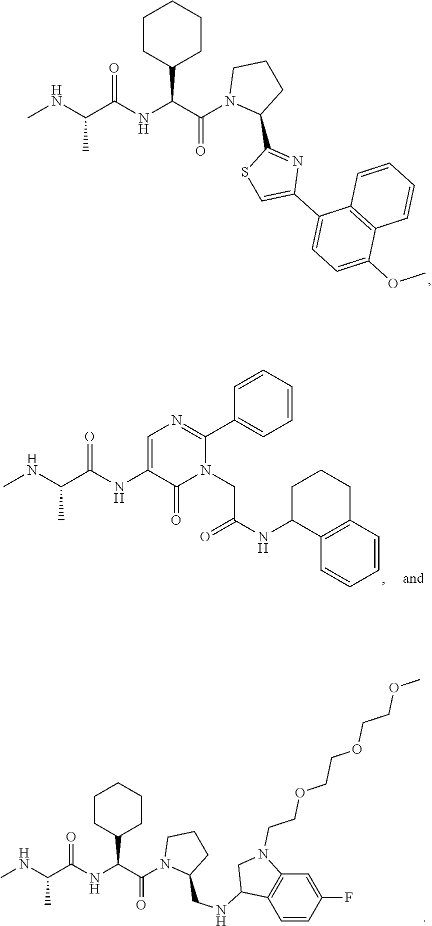

In any of the compounds described herein, the ILM can have the structure of Formula (XX), which is based on the IAP ligands described in WO Pub. No. 2007/101347, or an unnatural mimetic thereof:

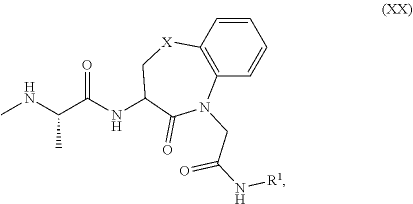

wherein X of Formula (XX) is selected from CH₂, O, NH, or S.

In any of the compounds described herein, the ILM can have the structure of Formula (XXI), which is based on the IAP ligands described in U.S. Pat. No. 7,345,081 and U.S. Pat. No. 7,419,975, or an unnatural mimetic thereof:

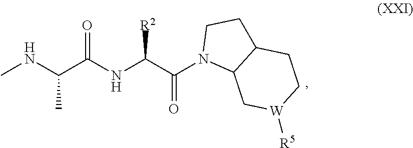

wherein:
R² of Formula (XXI) is selected from:

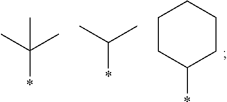

R⁵ of Formula (XXI) is selected from:

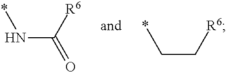

and
W of Formula (XXI) is selected from CH or N; and
R⁶ of

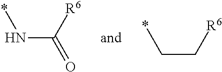

are independently a mono- or bicyclic fused aryl or heteroaryl.

In certain embodiments, the ILM of the compound is selected from the group consisting of:

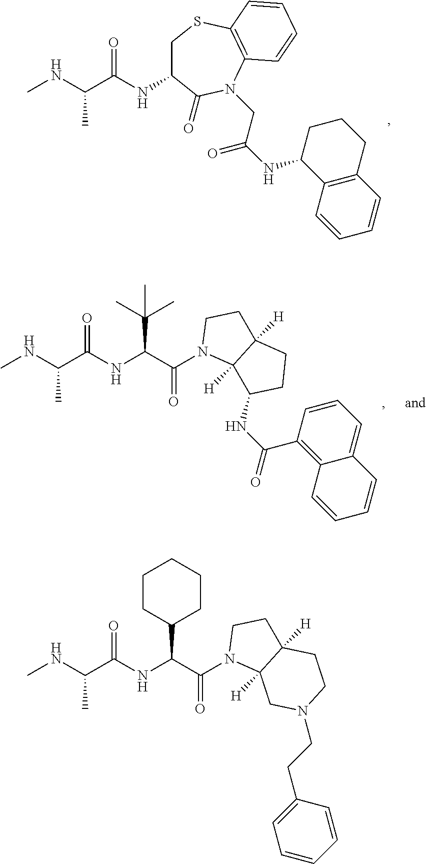 [SMILES: CN[C@@H](C)C(=O)N[C@@H]1CSc2ccccc2N(CC(=O)N[C@@H]2CCCc3ccccc32)C1=O ; CN[C@@H](C)C(=O)N[C@H](C(=O)N1CC[C@H]2CC[C@H](NC(=O)c3cccc4ccccc34)[C@H]21)C(C)(C)C ; CN[C@@H](C)C(=O)N[C@H](C(=O)N1CC[C@H]2CCN(CCc3ccccc3)C[C@H]21)C1CCCCC1]

In certain embodiments, the ILM of the compound is selected from the group consisting of:

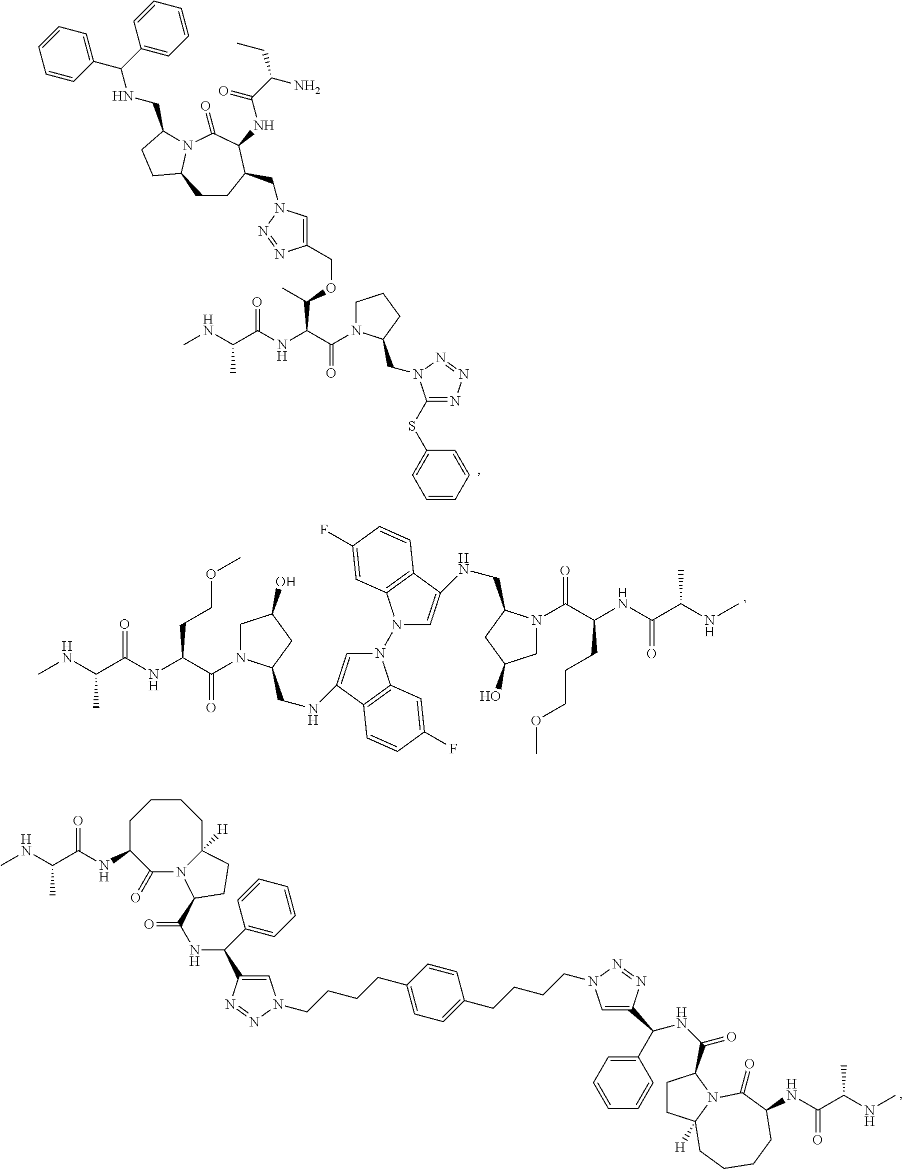

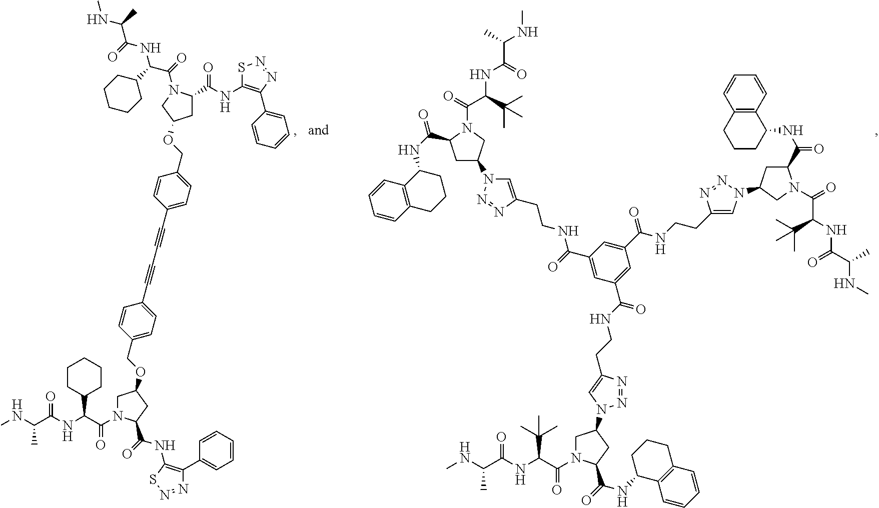 [SMILES: CN[C@@H](C)C(=O)N[C@H](C(=O)N1C[C@@H](OCc2ccc(C#CC#Cc3ccc(CO[C@H]4C[C@@H](C(=O)Nc5snnc5-c5ccccc5)N(C(=O)[C@@H](NC(=O)[C@H](C)NC)C5CCCCC5)C4)cc3)cc2)C[C@H]1C(=O)Nc1snnc1-c1ccccc1)C1CCCCC1 ; CN[C@@H](C)C(=O)N[C@H](C(=O)N1C[C@@H](n2cc(CCNC(=O)c3cc(C(=O)NCCc4cn([C@H]5C[C@@H](C(=O)N[C@@H]6CCCc7ccccc76)N(C(=O)[C@@H](NC(=O)[C@H](C)NC)C(C)(C)C)C5)nn4)cc(C(=O)NCCc4cn([C@H]5C[C@@H](C(=O)N[C@@H]6CCCc7ccccc76)N(C(=O)[C@@H](NC(=O)[C@H](C)NC)C(C)(C)C)C5)nn4)c3)nn2)C[C@H]1C(=O)N[C@@H]1CCCc2ccccc21)C(C)(C)C]

which are described in WO Publication No. 2009/060292, U.S. Pat. No. 7,517,906, WO Pub. No. 2008/134679, WO Pub. No. 2007/130626, and WO Pub. No. 2008/128121.

In any of the compounds described herein, the ILM can have the structure of Formula (XXII) or (XXIII), which are derived from the IAP ligands described in WO Pub. No. 2015/006524 and Perez H L, Discovery of potent heterodimeric antagonists of inhibitor of apoptosis proteins (IAPs) with sustained antitumor activity. J. Med. Chem. 58(3), 1556-62 (2015), or an unnatural mimetic thereof:

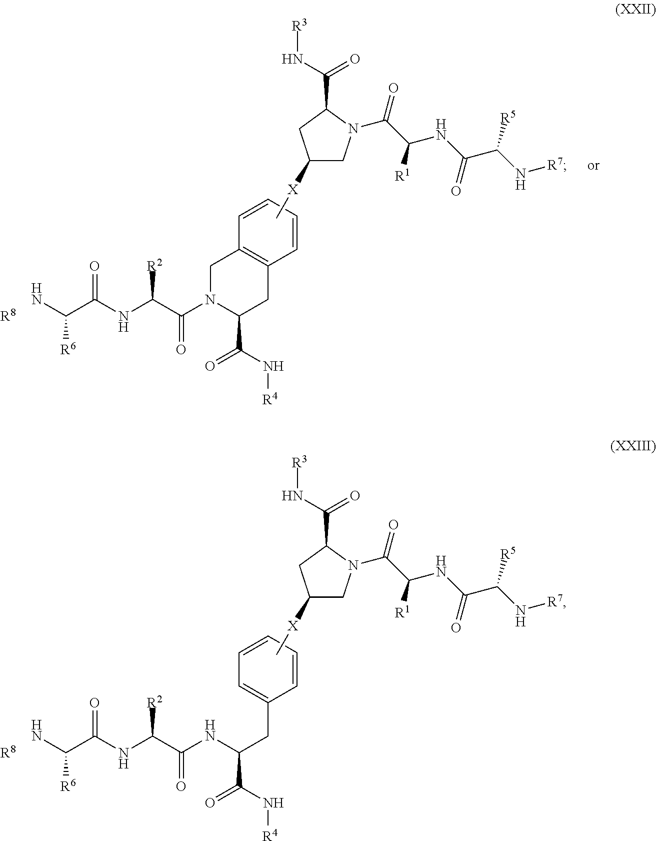

wherein:
R¹ of Formula (XXII) or (XXIII) is optionally substituted alkyl, optionally substituted cycloalkyl, optionally substituted cycloalkylalkyl, optionally substituted heterocyclyl, optionally substituted arylalkyl or optionally substituted aryl;
R² of Formula (XXII) or (XXIII) is optionally substituted alkyl, optionally substituted cycloalkyl, optionally substituted cycloalkylalkyl, optionally substituted heterocyclyl, optionally substituted arylalkyl or optionally substituted aryl;
or alternatively, R¹ and R² of Formula (XXII) or (XXIII) are independently optionally substituted thioalkyl wherein the substituents attached to the S atom of the thioalkyl are optionally substituted alkyl, optionally substituted branched alkyl, optionally substituted heterocyclyl, —(CH₂)ᵥCOR²⁰, —CH₂CHR²¹COR²² or —CH₂R²³;
wherein:
v is an integer from 1-3;
R²⁰ and R²² of —(CH₂)ᵥCOR²⁰ and —CH₂R²³ are independently selected from OH, NR²⁴R²⁵ or OR²⁶;
R²¹ of —CH₂CHR²¹COR² is selected from the group NR²⁴R²⁵;
R²³ of —CH₂R²³ is selected from optionally substituted aryl or optionally substituted heterocyclyl, where the optional substituents include alkyl and halogen;
R²⁴ of NR²⁴R²⁵ is selected from hydrogen or optionally substituted alkyl;
R²⁵ of NR²⁴R²⁵ is selected from hydrogen, optionally substituted alkyl, optionally substituted branched alkyl, optionally substituted arylalkyl, optionally substituted heterocyclyl, —CH₂(OCH₂CH₂O)ₘCH₃, or a polyamine chain, such as spermine or spermidine;
R²⁶ of OR²⁶ is selected from optionally substituted alkyl, wherein the optional substituents are OH, halogen or NH₂; and
m is an integer from 1-8;
R³ and R⁴ of Formula (XXII) or (XXIII) are independently selected from optionally substituted alkyl, optionally substituted cycloalkyl, optionally substituted aryl, optionally substituted arylalkyl, optionally substituted arylalkoxy, optionally substituted heteroaryl, optionally substituted heterocyclyl, optionally substituted heteroarylalkyl or optionally substituted heterocycloalkyl, wherein the substituents are alkyl, halogen or OH;
R⁵, R⁶, R⁷ and R⁸ of Formula (XXII) or (XXIII) are independently selected from hydrogen, optionally substituted alkyl or optionally substituted cycloalkyl; and
X is selected from a bond or a chemical linker group, and/or a pharmaceutically acceptable salt, tautomer or stereoisomer thereof.

In certain embodiments, X is a bond or is selected from the group consisting of:

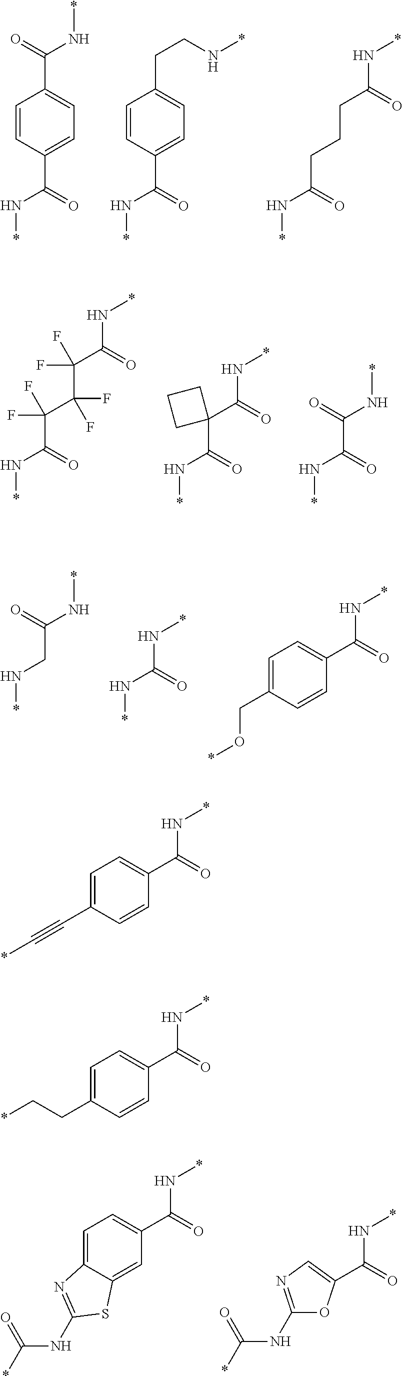

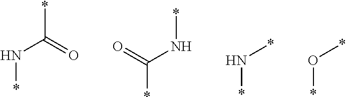

wherein “*” is the point of attachment of a PTM, L or ULM, e.g., an ILM.

In any of the compounds described herein, the ILM can have the structure of Formula (XXIV) or (XXVI), which are derived from the IAP ligands described in WO Pub. No. 2015/006524 and Perez H L, Discovery of potent heterodimeric antagonists of inhibitor of apoptosis proteins (IAPs) with sustained antitumor activity. J. Med. Chem. 58(3), 1556-62 (2015), or an unnatural mimetic thereof, and the chemical linker to linker group L as shown:

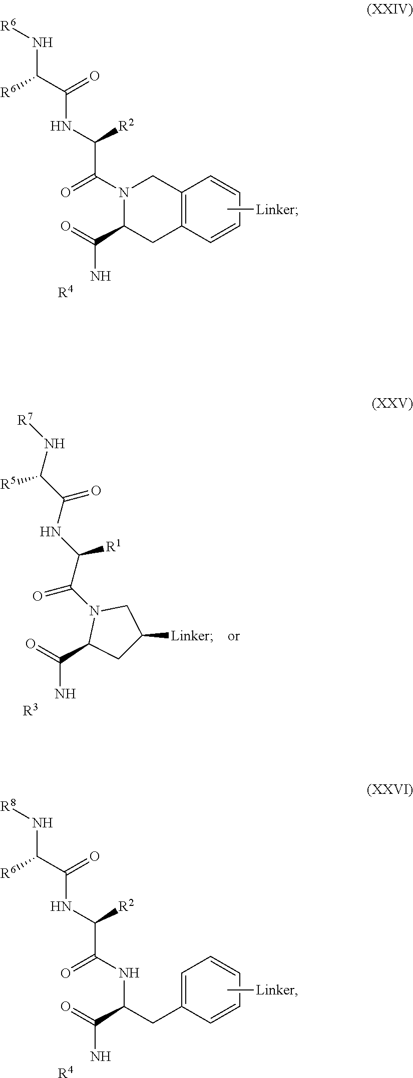

wherein:
R¹ of Formula (XXIV), (XXV) or (XXVI) is selected from optionally substituted alkyl, optionally substituted cycloalkyl, optionally substituted cycloalkylalkyl, optionally substituted heterocyclyl, optionally substituted arylalkyl or optionally substituted aryl;
R² of Formula (XXIV), (XXV) or (XXVI) is selected from optionally substituted alkyl, optionally substituted cycloalkyl, optionally substituted cycloalkylalkyl, optionally substituted heterocyclyl, optionally substituted arylalkyl or optionally substituted aryl;
or alternatively,
R¹ and R² of Formula (XXIV), (XXV) or (XXVI) are independently selected from optionally substituted thioalkyl wherein the substituents attached to the S atom of the thioalkyl are optionally substituted alkyl, optionally substituted branched alkyl, optionally substituted heterocyclyl, —(CH₂)ᵥCOR²⁰, —CH₂CHR²¹COR²² or —CH₂R²³,
wherein:
   v is an integer from 1-3;
   R²⁰ and R²² of —(CH₂)ᵥCOR²⁰ and —CH₂R²³ are independently selected from OH, NR²⁴R²⁵ or OR²⁶;
   R²¹ of —CH₂CHR²¹COR² is selected from NR²⁴R²⁵;
   R²³ of —CH₂R²³ is selected from optionally substituted aryl or optionally substituted heterocyclyl, wherein the optional substituents include alkyl and halogen;
   R²⁴ of NR²⁴R²⁵ is selected from hydrogen or optionally substituted alkyl;
   R²⁵ of NR²⁴R²⁵ is selected from hydrogen, optionally substituted alkyl, optionally substituted branched alkyl, optionally substituted arylalkyl, optionally substituted heterocyclyl, —CH₂(OCH₂CH₂O)ₘCH₃, or a polyamine chain, such as spermine or spermidine;
   R²⁶ of OR²⁶ is selected from optionally substituted alkyl, wherein the optional substituents are OH, halogen or NH₂; and
   m is an integer from 1-8;
   R³ and R⁴ of Formula (XXIV), (XXV) or (XXVI) are independently optionally substituted alkyl, optionally substituted cycloalkyl, optionally substituted aryl, optionally substituted arylalkyl, optionally substituted arylalkoxy, optionally substituted heteroaryl, optionally substituted heterocyclyl, optionally substituted heteroarylalkyl or optionally substituted heterocycloalkyl, wherein the substituents are alkyl, halogen or OH;
   R⁵, R⁶, R⁷ and R⁸ of Formula (XXIV), (XXV) or (XXVI) are independently hydrogen, optionally substituted alkyl or optionally substituted cycloalkyl; and/or a pharmaceutically acceptable salt, tautomer or stereoisomer thereof.

In a particular embodiment, the ILM according to Formulas (XXII) through (XXVI):

R⁷ and R⁸ are selected from the H or Me;
R⁵ and R⁶ are selected from the group comprising:

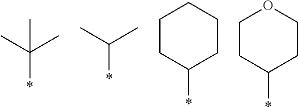

R³ and R⁴ are selected from the group comprising:

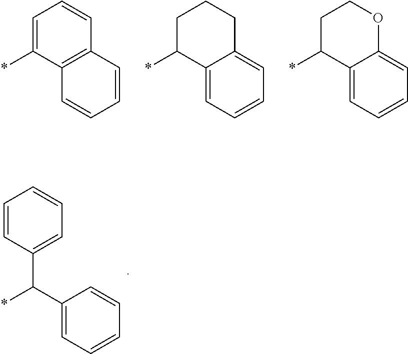

In any of the compounds described herein, the ILM can have the structure of Formula (XXVII) or (XXVII), which are derived from the IAP ligands described in WO Pub. No. 2014/055461 and Kim, K S, Discovery of tetrahydroisoquinoline-based bivalent heterodimeric IAP antagonists. Bioorg. Med. Chem. Lett. 24(21), 5022-9 (2014), or an unnatural mimetic thereof:

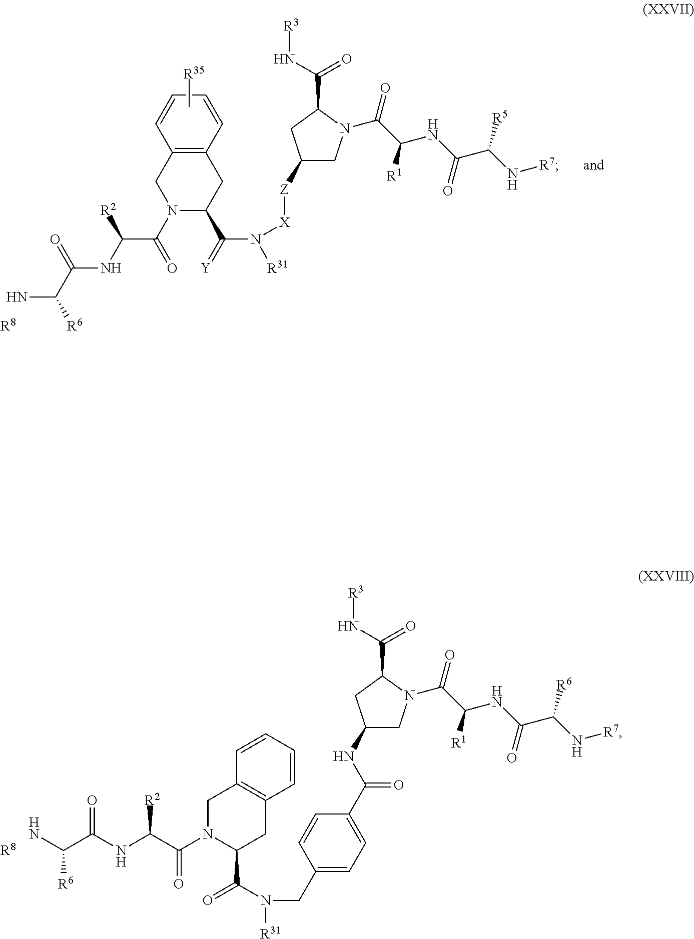

wherein:
R³⁵ is 1-2 substituents selected from alkyl, halogen, alkoxy, cyano and haloalkoxy;
R¹ of Formula (XXVII) and (XXVIII) is selected from H or an optionally substituted alkyl, optionally substituted cycloalkyl, optionally substituted cycloalkylalkyl, optionally substituted heterocyclyl, optionally substituted arylalkyl or optionally substituted aryl;
R² of Formula (XXVII) and (XXVIII) is selected from H or an optionally substituted alkyl, optionally substituted cycloalkyl, optionally substituted cycloalkylalkyl, optionally substituted heterocyclyl, optionally substituted arylalkyl or optionally substituted aryl;
or alternatively,
R¹ and R² of Formula (XXVII) and (XXVIII) are independently selected from an optionally substituted thioalkyl —CR⁶⁰R⁶¹SR⁷⁰, wherein R⁶⁰ and R⁶¹ are selected from H or methyl, and R⁷⁰ is selected from an optionally substituted alkyl, optionally substituted branched alkyl, optionally substituted heterocyclyl, —(CH₂)ᵥCOR²⁰, —CH₂CHR²¹COR²² or —CH₂R²³,
wherein:
   v is an integer from 1-3;
   R²⁰ and R²² of —(CH₂)ᵥCOR²⁰ and —CH₂CHR²¹COR²² are independently selected from OH, NR²⁴R²⁵ or OR²⁶;
   R²¹ of —CH₂CHR²¹COR²² is selected from NR²⁴R²⁵;
   R²³ of —CH₂R²³ is selected from an optionally substituted aryl or optionally substituted heterocyclyl, where the optional substituents include alkyl and halogen;
   R²⁴ of NR²⁴R²⁵ is selected from hydrogen or optionally substituted alkyl;
   R²⁵ of NR²⁴R²⁵ is selected from hydrogen, optionally substituted alkyl, optionally substituted branched alkyl, optionally substituted arylalkyl, optionally substituted heterocyclyl, —CH₂CH₂(OCH₂CH₂)ₘCH₃, or a polyamine chain —[CH₂CH₂(CH₂)_{δ}NH]_{ψ}CH₂CH₂(CH₂) [symbol] NH₂, such as spermine or spermidine;
   wherein δ=0-2, ψ=1-3, [symbol] =0-2;
   R²⁶ of OR²⁶ is an optionally substituted alkyl, wherein the optional substituents are OH, halogen or NH₂; and
   m is an integer from 1-8,
   R³ and R⁴ of Formula (XXVII) and (XXVIII) are independently selected from an optionally substituted alkyl, optionally substituted cycloalkyl, optionally substituted aryl, optionally substituted arylalkyl, optionally substituted arylalkoxy, optionally substituted heteroaryl, optionally substituted heterocyclyl, optionally substituted heteroarylalkyl or optionally substituted heterocycloalkyl, wherein the substituents are alkyl, halogen or OH;
   R⁵, R⁶, R⁷ and R⁸ of Formula (XXVII) and (XXVIII) are independently selected from hydrogen, optionally substituted alkyl or optionally substituted cycloalkyl;
   R³¹ of Formulas (XXVII) and (XXVIII) is selected from alkyl, aryl, arylalkyl, heteroaryl or heteroarylalkyl optionally further substituted, preferably selected form the group consisting of:

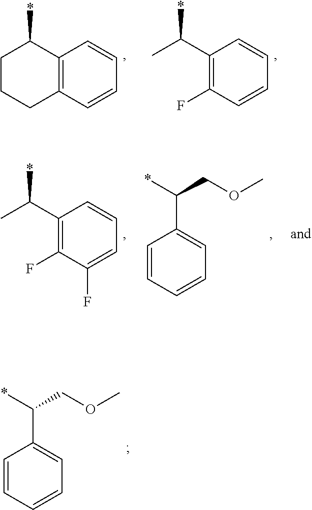

X of Formulas (XXVII) and (XXVIII) is selected from —(CR⁸¹R⁸²)ₘ—, optionally substituted heteroaryl or heterocyclyl,

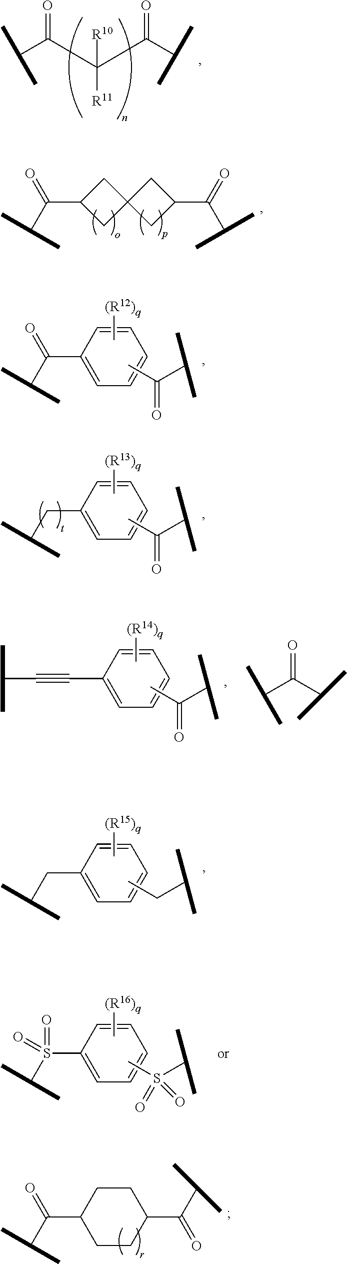

Z of Formulas (XXVII) is selected from C═O, —O—, —NR, —CONH—, —NHCO—, or may be absent;
R⁸¹ and R⁸² of —(CR⁸¹R⁸²)ₘ— are independently selected from hydrogen, halogen, alkyl or cycloalkyl, or R⁸¹ and R⁸² can be taken together to form a carbocyclic ring;
R¹⁰ and R¹¹ of

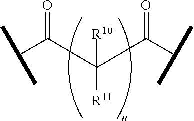

are independently selected from hydrogen, halogen or alkyl;
R¹², R¹³, R¹⁴, R¹⁵ and R¹⁶ of

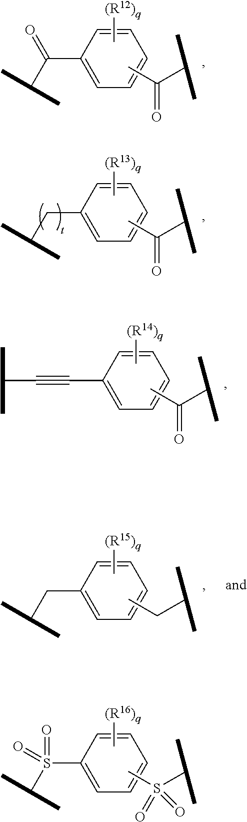

are independently selected from hydrogen, halogen or optionally substituted alkyl or OR¹⁷;
R¹⁷ is selected from hydrogen, optionally substituted alkyl or optionally substituted cycloalkyl;
m and n of —(CR²¹R²²)ₘ— and

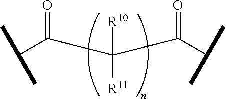

are independently 0, 1, 2, 3, or 4;
o and p of

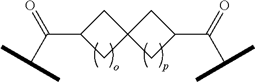

are independently 0, 1, 2 or 3;
q and t of

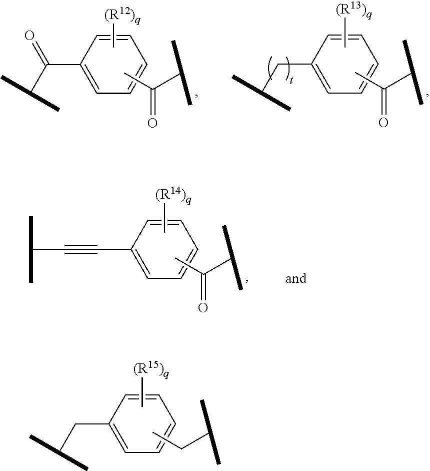

are independently 0, 1, 2, 3, or 4;
r of

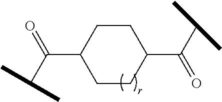

is 0 or 1;
and/or a pharmaceutically acceptable salt, tautomer or stereoisomer thereof.

In any of the compounds described herein, the ILM can have the structure of Formula (XXIX), (XXX), (XXXI), or (XXXII), which are derived from the IAP ligands described in WO Pub. No. 2014/055461 and Kim, K S, Discovery of tetrahydroisoquinoline-based bivalent heterodimeric IAP antagonists. Bioorg. Med. Chem. Lett. 24(21), 5022-9 (2014), or an unnatural mimetic thereof, and the chemical linker to linker group L as shown:

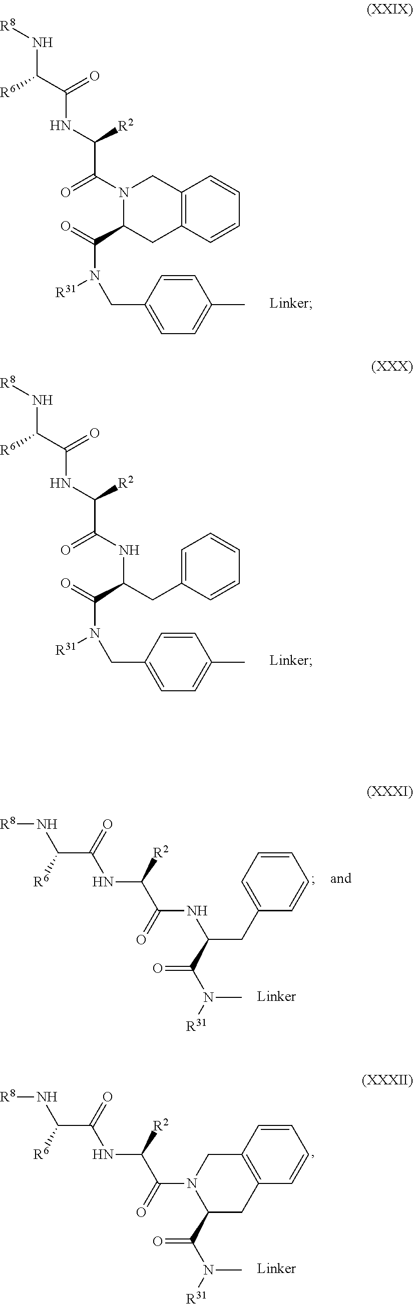

wherein:
R² of Formula (XXIX) through (XXXII) is selected from H, an optionally substituted alkyl, optionally substituted cycloalkyl, optionally substituted cycloalkylalkyl, optionally substituted heterocyclyl, optionally substituted arylalkyl or optionally substituted aryl;
or alternatively;
R¹ and R² of Formula (XXVII) and (XXVIII) are independently selected from H, an optionally substituted thioalkyl —CR⁶⁰R⁶¹SR⁷⁰ wherein R⁶⁰ and R⁶¹ are selected from H or methyl, and R⁷⁰ is an optionally substituted alkyl, optionally substituted branched alkyl, optionally substituted heterocyclyl, —(CH₂)ᵥCOR²⁰, —CH₂CHR²¹COR²² or —CH₂R²³;
wherein:
v is an integer from 1-3;
R²⁰ and R²² of —(CH₂)ᵥCOR²⁰ and —CH₂CHR²¹COR²² are independently selected from OH, NR²⁴R²⁵ or OR²⁶;
R²¹ of —CH₂CHR²¹COR²² is selected from NR²⁴R²⁵;
R²³ of —CH₂R²³ is selected from an optionally substituted aryl or optionally substituted heterocyclyl, where the optional substituents include alkyl and halogen;
R²⁴ of NR²⁴R²⁵ is selected from hydrogen or optionally substituted alkyl;
R²⁵ of NR²⁴R²⁵ is selected from hydrogen, optionally substituted alkyl, optionally substituted branched alkyl, optionally substituted arylalkyl, optionally substituted heterocyclyl, —CH₂CH₂(OCH₂CH₂)ₘCH₃, or a polyamine chain —[CH₂CH₂(CH₂)_{δ}NH]_{ψ}CH₂CH₂(CH₂) [symbol] ᵣNH₂, such as spermine or spermidine,
wherein δ=0-2, ψ=1-3, [symbol] =0-2;
R²⁶ of OR²⁶ is an optionally substituted alkyl, wherein the optional substituents are OH, halogen or NH₂,
m is an integer from 1-8;
R⁶ and R⁸ of Formula (XXIX) through (XXXII) are independently selected from hydrogen, optionally substituted alkyl or optionally substituted cycloalkyl; and
R³¹ of Formulas (XXIX) through (XXXII) is selected from alkyl, aryl, arylalkyl, heteroaryl or heteroarylalkyl optionally further substituted, preferably selected form the group consisting of:

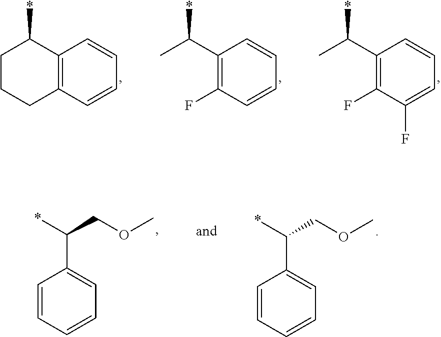

In certain embodiments, the ILM of the compound is:

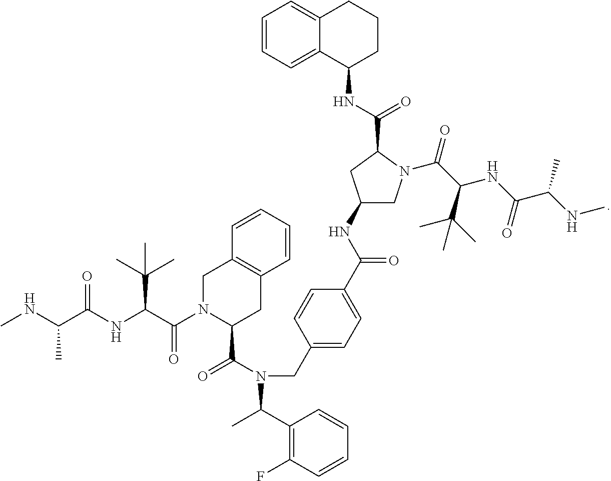

In any of the compounds described herein, the ILM can have the structure of Formula (XXXIII), which are derived from the IAP ligands described in WO Pub. No. 2014/074658 and WO Pub. No. 2013/071035, or an unnatural mimetic thereof:

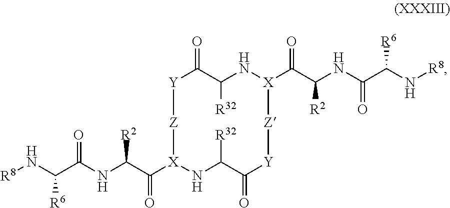

wherein:
R² of Formula (XXXIII) is selected from H, an optionally substituted alkyl, optionally substituted cycloalkyl, optionally substituted cycloalkylalkyl, optionally substituted heterocyclyl, optionally substituted arylalkyl or optionally substituted aryl;
R⁶ and R⁸ of Formula (XXXIII) are independently selected from hydrogen, optionally substituted alkyl or optionally substituted cycloalkyl;
R³² of Formula (XXXIII) is selected from (C1-C4 alkylene)-R³³ wherein R³³ is selected from hydrogen, aryl, heteroaryl or cycloalkyl optionally further substituted;
X of Formula (XXXIII) is selected from:

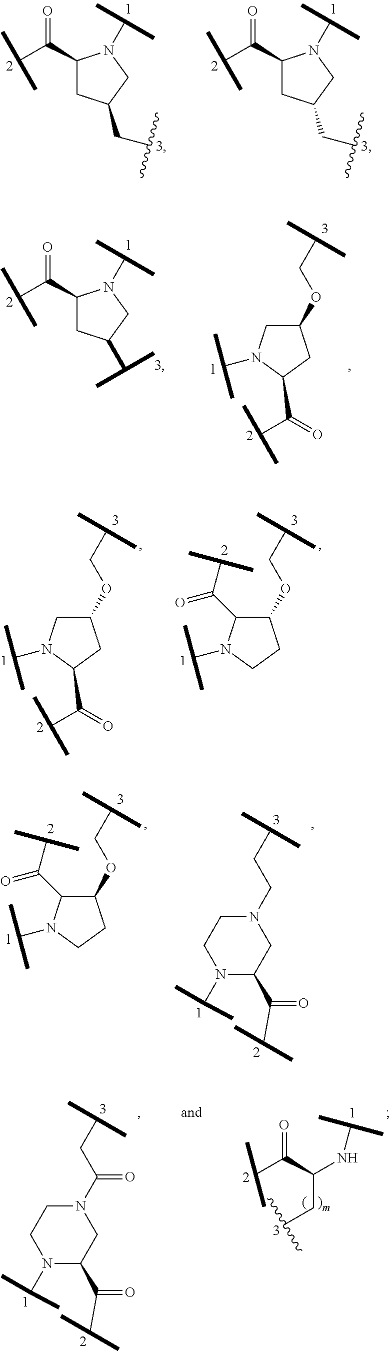

Z and Z′ of Formula (XXXIII) are independently selected from:

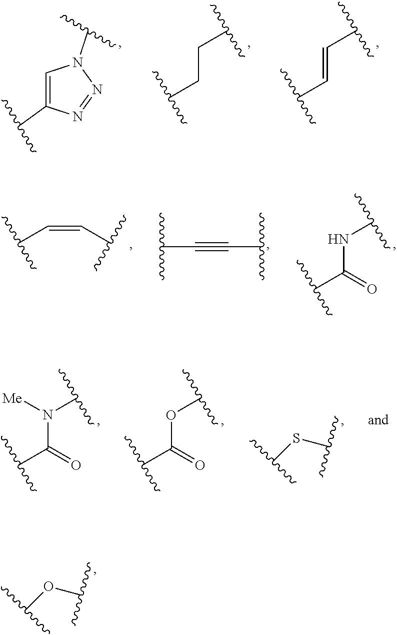

wherein each

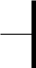

represents a point of attachment to the compound, and Z and Z′ cannot both be

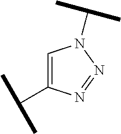

in any given compound;
Y of Formula (XXXIII) is selected from:

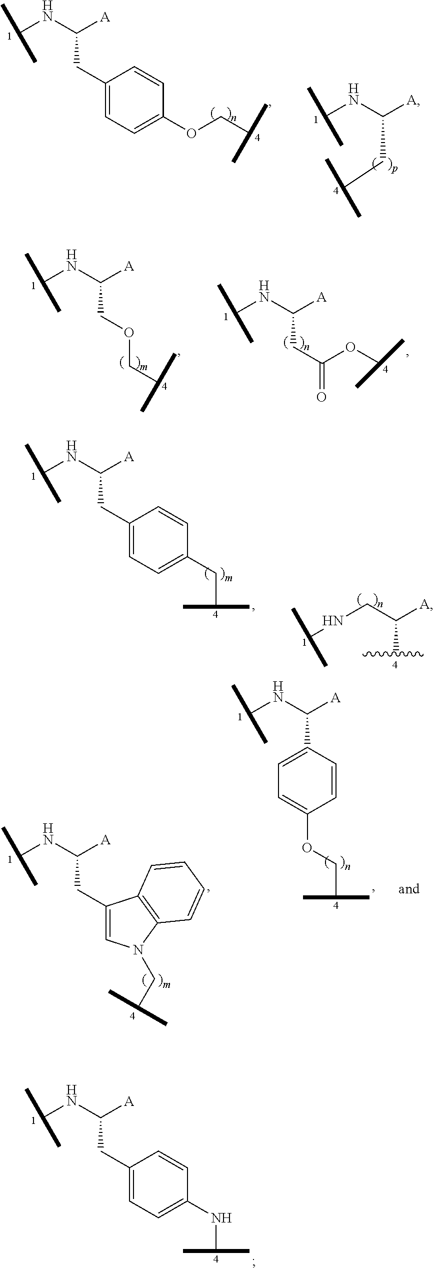

wherein Z and Z′ of Formula (XXXIII) are the same and Z is

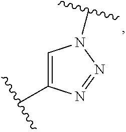 [SMILES: *c1cn(*)nn1]

wherein each

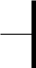

represents a point of attachment to the compound,
X is selected from:

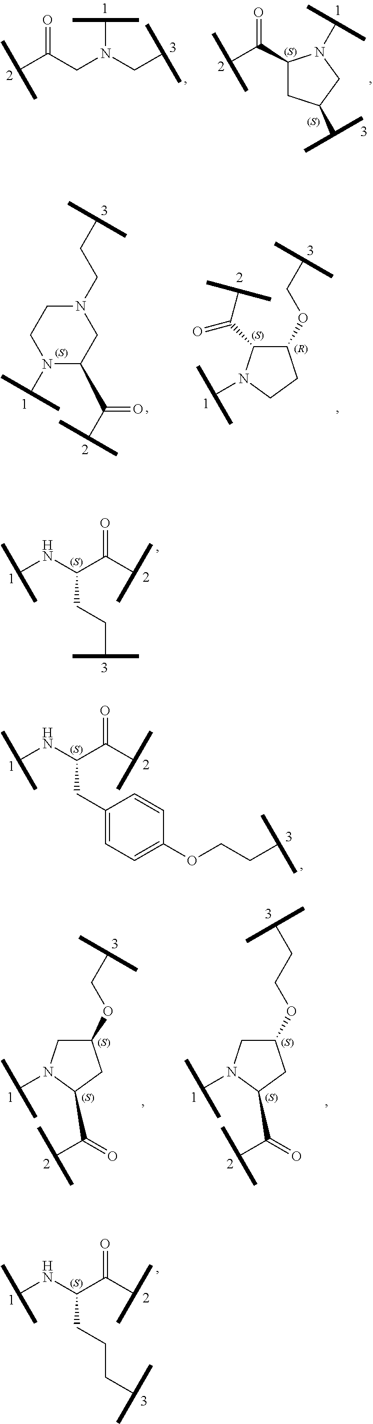

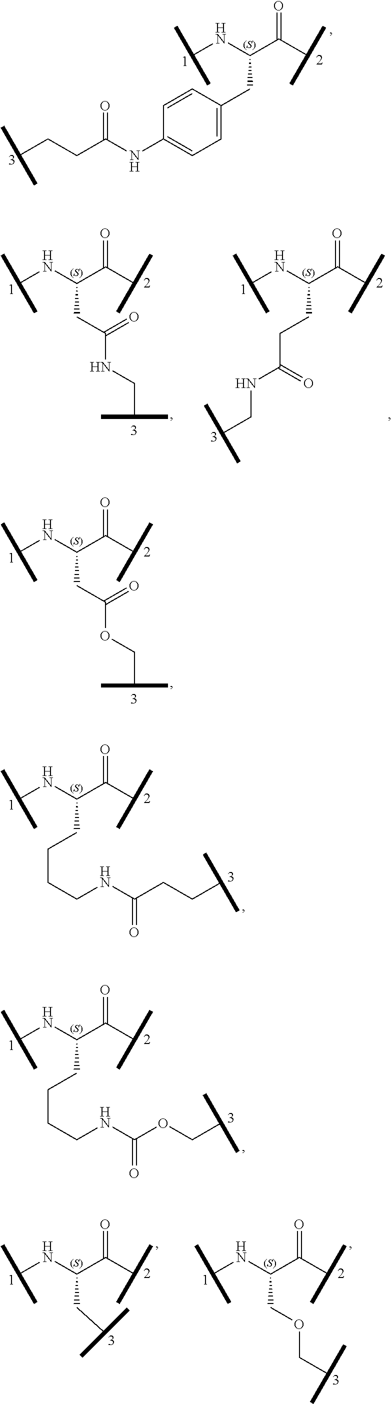

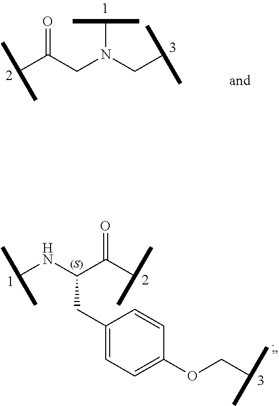

and
Y of Formula (XXXIII) is independently selected from:

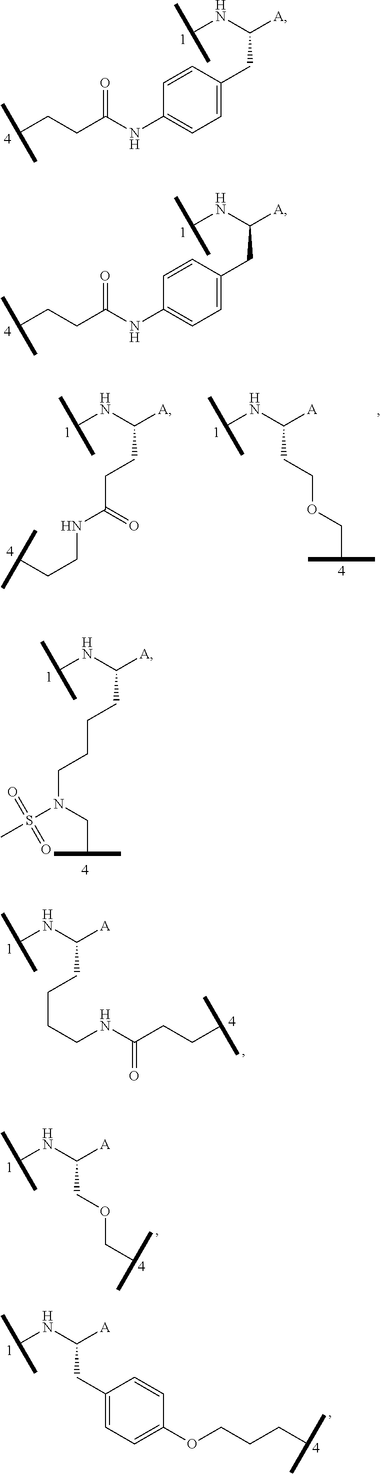

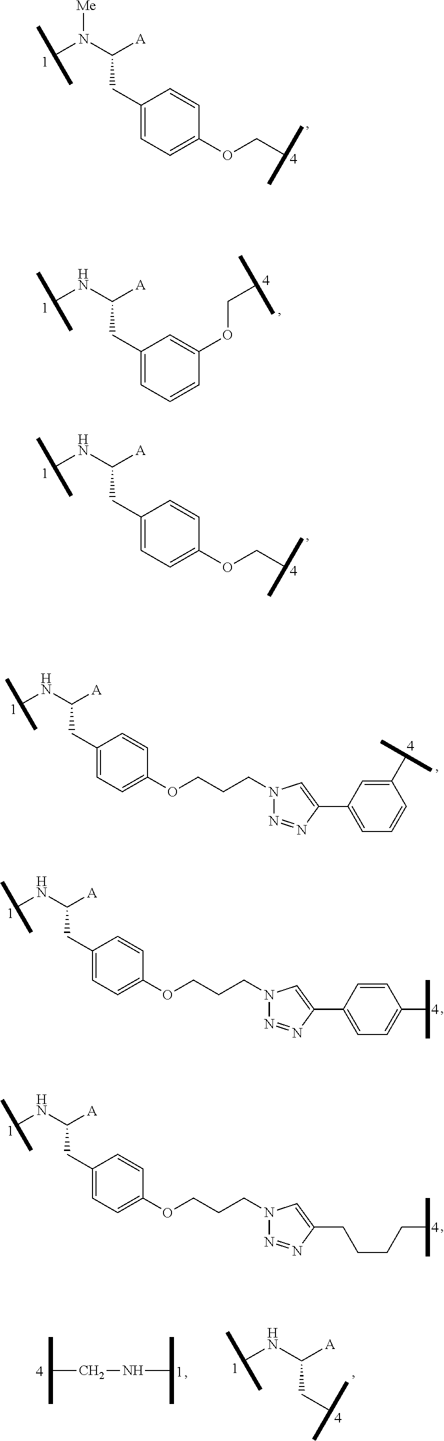

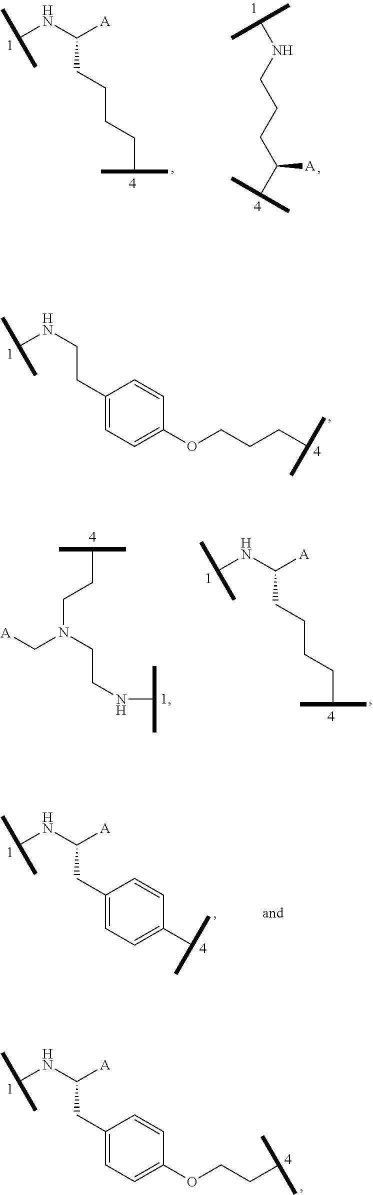

wherein:

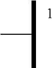

represents a point of attachment to a —C═O portion of the compound;

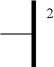

represents a point of attachment to a —NH portion of the compound;

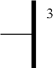

represents a first point of attachment to Z;

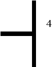

represents a second point of attachment to Z;
m is an integer from 0-3;
n is an integer from 1-3;
p is an integer from 0-4; and
A is —C(O)R³;
R³ is selected from —C(O)R³ is OH, NHCN, NHSO₂R¹⁰, NHOR¹¹ or N(R¹²)(R¹³);
R¹⁰ and F¹¹ of NHSO₂R¹⁰ and NHOR¹¹ are independently selected from hydrogen, optionally substituted —C₁-C₄ alkyl, cycloalkyl, aryl, heteroaryl, heterocyclyl or heterocycloalkyl;
R¹² and R¹³ of N(R¹²)(R¹³) are independently selected from hydrogen, —C₁-C₄ alkyl, —(C₁-C₄) alkylene)-NH—(C₁-C₄ alkyl), and —(C₁-C₄ alkylene)-O—(C₁-C₄ hydroxyalkyl), or R¹² and R¹³ taken together with the nitrogen atom to which they are commonly bound to form a saturated heterocyclyl optionally comprising one additional heteroatom selected from N, O and S, and wherein the saturated heterocycle is optionally substituted with methyl.

In any of the compounds described herein, the ILM can have the structure of Formula (XXXIV) or (XXXV), which are derived from the IAP ligands described in WO Pub. No. 2014/047024, or an unnatural mimetic thereof:

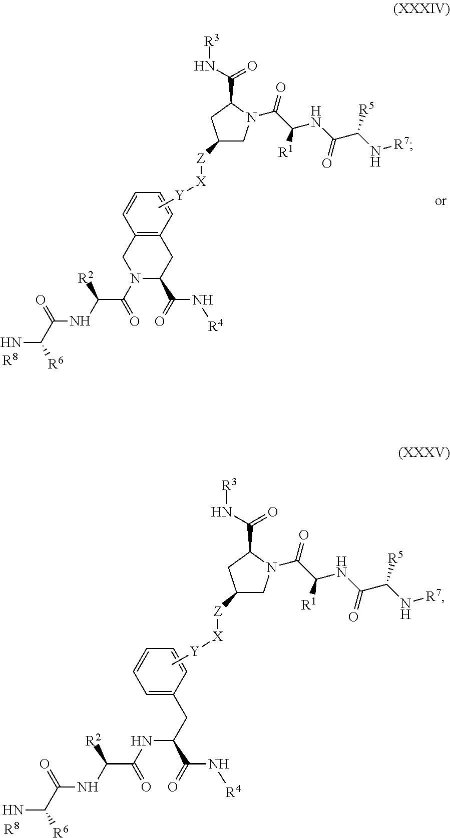

wherein:
X of Formula (XXXIV) or (XXXV) is absent or a group selected from —(CR¹⁰R¹¹)ₘ—, optionally substituted heteroaryl or optionally substituted heterocyclyl,

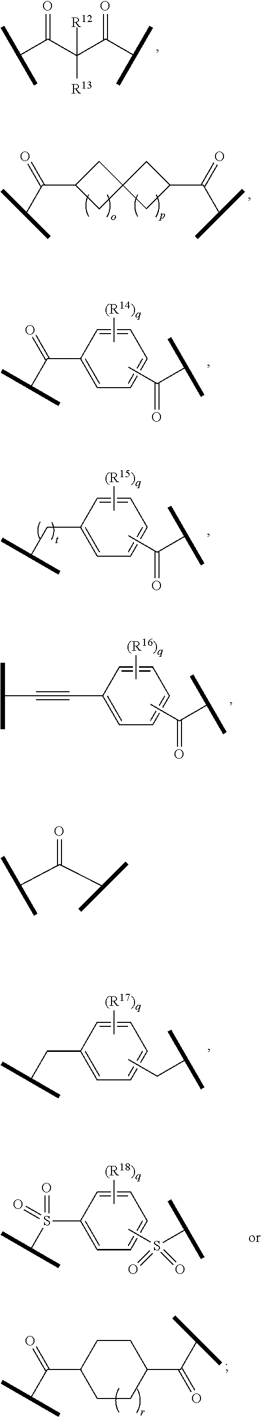

Y and Z of Formula (XXXIV) or (XXXV) are independently selected from C═O, —O—, —NR⁹—, —CONH—, —NHCO— or may be absent;
R¹ and R² of Formula (XXXIV) or (XXXV) are independently selected from an optionally substituted alkyl, optionally substituted cycloalkyl, optionally substituted cycloalkylalkyl, optionally substituted arylalkyl, optionally substituted aryl, or
R¹ and R² of Formula (XXXIV) or (XXXV) are independently selected from optionally substituted thioalkyl wherein the substituents attached to the S atom of the thioalkyl are optionally substituted alkyl, optionally substituted branched alkyl, optionally substituted heterocyclyl, —(CH₂)ᵥCOR²⁰, —CH₂CHR²¹COR²² or —CH₂R²³; wherein
   v is an integer from 1-3;
   R²⁰ and R²² of —(CH₂)ᵥCOR²⁰ and —CH₂CHR²¹COR²² are independently selected from OH, NR²⁴R²⁵ or OR²⁶;
   R²¹ of —CH₂CHR²¹COR²² is selected from NR²⁴R²⁵;
   R²³ of —CH₂R²³ are selected from an optionally substituted aryl or optionally substituted heterocyclyl, where the optional substituents include alkyl and halogen;
   R²⁴ of NR²⁴R²⁵ is selected from hydrogen or optionally substituted alkyl;
   R²⁵ of NR²⁴R²⁵ is selected from hydrogen, optionally substituted alkyl, optionally substituted branched alkyl, optionally substituted arylalkyl, optionally substituted heterocyclyl, —CH₂(OCH₂CH²⁰)mCH3, or a polyamine chain;
   R²⁶ is an optionally substituted alkyl, wherein the optional substituents are OH, halogen or NH₂;
   m of —(CR¹⁰R¹¹)ₘ— is an integer from 1-8;
R³ and R⁴ of Formula (XXXIV) or (XXXV) are independently selected from optionally substituted alkyl, optionally substituted cycloalkyl, optionally substituted aryl, optionally substituted arylalkyl, optionally substituted arylalkoxy, optionally substituted heteroaryl, optionally substituted heterocyclyl, optionally substituted heteroarylalkyl or optionally substituted heterocycloalkyl, wherein the substituents are alkyl, halogen or OH;
R⁵, R⁶, R⁷ and R⁸ of Formula (XXXIV) or (XXXV) are independently selected from hydrogen, optionally substituted alkyl or optionally substituted cycloalkyl;

R¹⁰ and R¹¹ of —(CR¹⁰R¹¹)ₘ— are independently selected from hydrogen, halogen or optionally substituted alkyl;
R¹² and R¹³ of

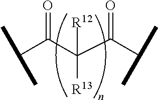

are independently selected from hydrogen, halogen or optionally substituted alkyl, or R¹² and R¹³ can be taken together to form a carbocyclic ring;
R¹⁴, R¹⁵, R¹⁶, R¹⁷ and R¹⁸ of

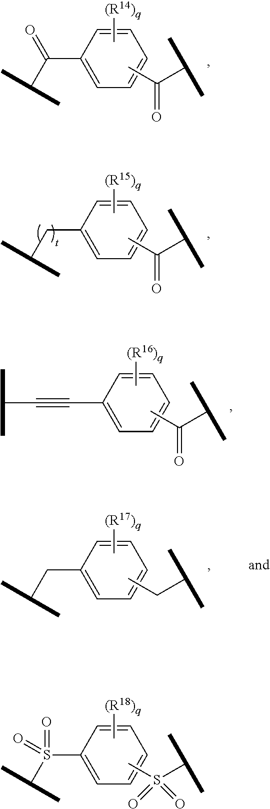

are independently selected from hydrogen, halogen, optionally substituted alkyl or OR¹⁹;
R¹⁹ of OR¹⁹ is selected from hydrogen, optionally substituted alkyl or optionally substituted cycloalkyl;
m and n of —(CR¹⁰R¹¹)ₘ— are independently 0, 1, 2, 3, or 4;
o and p of —(CR¹⁰R¹¹)ₘ— are independently 0, 1, 2 or 3;
q of —(CR¹⁰R¹¹)ₘ— is 0, 1, 2, 3, or 4; r is 0 or 1;
t of —(CR¹⁰R¹¹)ₘ— is 1, 2, or 3; and/or a pharmaceutically acceptable salt, tautomer or stereoisomer thereof.

In any of the compounds described herein, the ILM can have the structure of Formula (XXXVI), which are derived from the IAP ligands described in WO Pub. No. 2014/025759, or an unnatural mimetic thereof:

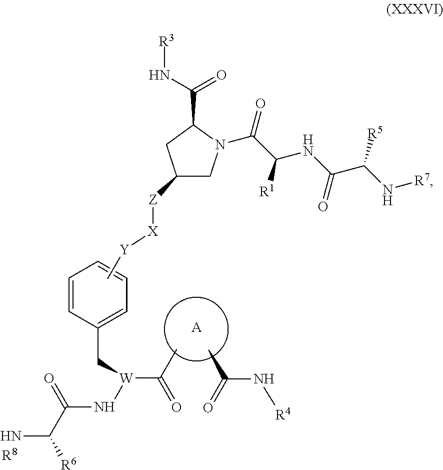

where:
A of Formula (XXXVI) is selected from:

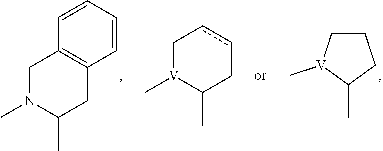

where the dotted line represents an optional double bond;
X of Formula (XXXVI) is selected from: —(CR²¹R²²)ₘ—,

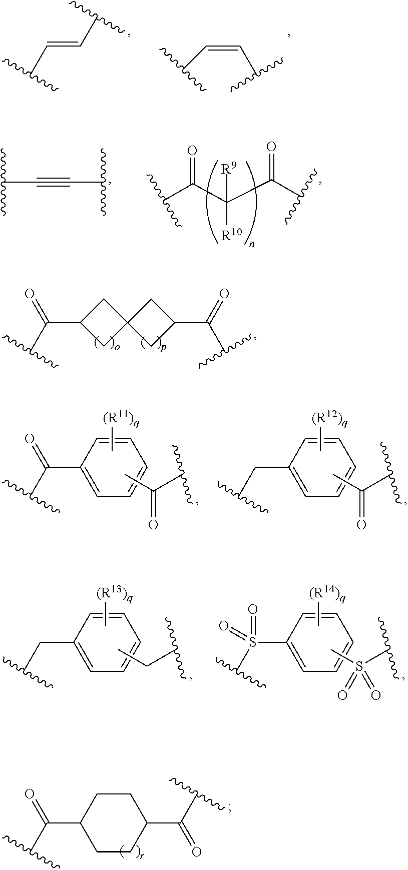

Y and Z of Formula (XXXVI) are independently selected from —O—, —NR⁶— or are absent;
V of Formula (XXXVI) is selected from —N— or —CH—;
W of Formula (XXXVI) is selected from —CH— or —N—;
R¹ of Formula (XXXVI) is selected from an optionally substituted alkyl, optionally substituted cycloalkyl, optionally substituted cycloalkylalkyl, optionally substituted arylalkyl or optionally substituted aryl;
R³ and R⁴ of Formula (XXXVI) are independently selected from optionally substituted alkyl, optionally substituted cycloalkyl, optionally substituted aryl, optionally substituted heteroaryl, optionally substituted heterocyclyl, optionally substituted arylalkyl, optionally substituted heteroarylalkyl or optionally substituted heterocycloalkyl;
R⁵, R⁶, R⁷ and R⁸ of Formula (XXIV), (XXV) or (XXVI) are independently selected from hydrogen, optionally substituted alkyl or optionally substituted cycloalkyl, or preferably methyl;
R⁹ and R¹⁰ of

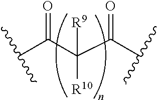

are independently selected from hydrogen, halogen or optionally substituted alkyl, or R⁹ and R¹⁰ can be taken together to form a ring;
R¹¹, R¹², R¹³ and R¹⁴ of

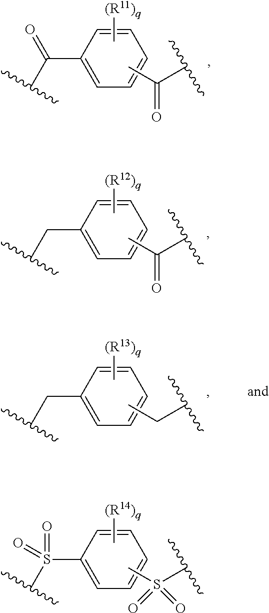

are independently selected from hydrogen, halogen, optionally substituted alkyl or OR¹⁵;
R¹⁵ of OR¹⁵ is selected from hydrogen, optionally substituted alkyl or optionally substituted cycloalkyl;
m and n of —(CR²¹R²²)ₘ— and

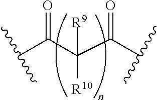

are independently selected from 0, 1, 2, 3, or 4;
o and p of

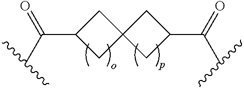

and are independently selected from 0, 1, 2 or 3;
q of

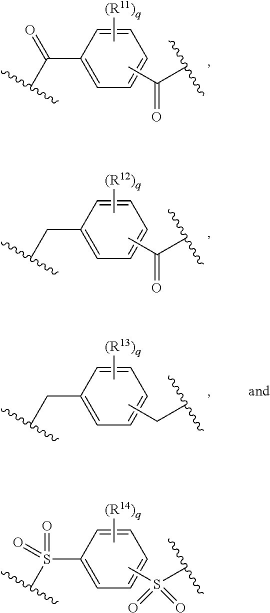

is selected from 0, 1, 2, 3, or 4;
r of

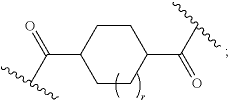

is selected from 0 or 1, and/or or a pharmaceutically acceptable salt, tautomer or stereoisomer thereof.

In any of the compounds described herein, the ILM can have the structure of Formula (XXXVII) or (XXXVIII), which are derived from the IAP ligands described in WO Pub. No. 2014/011712, or an unnatural mimetic thereof:

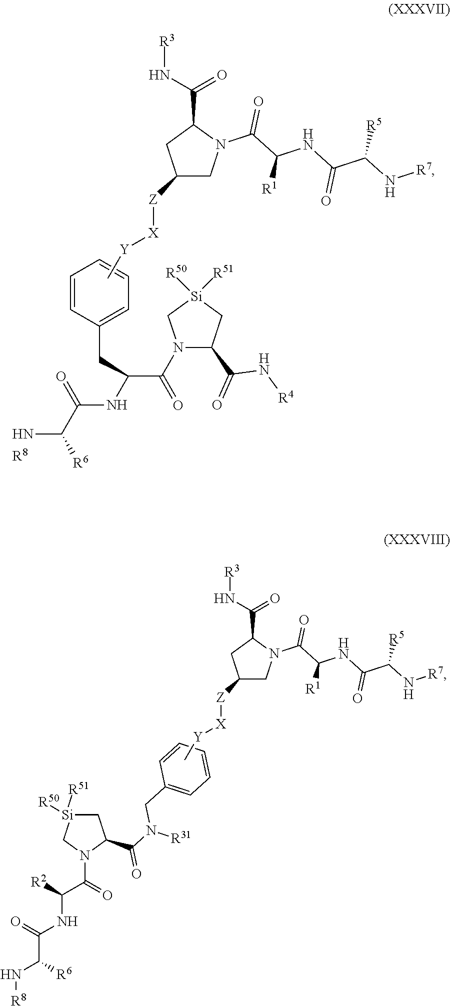

wherein:
X of Formulas (XXXVII) and (XXXVIII) is —(CR¹⁶R¹⁷)ₘ—,

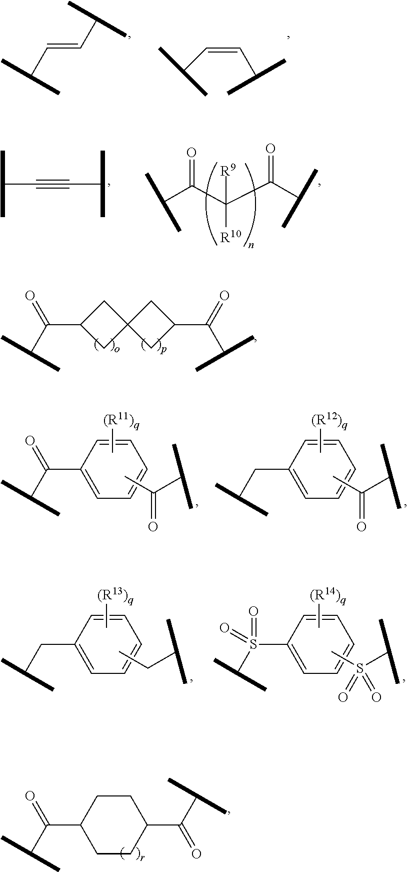

or absent;
Y and Z of Formula (XXXVII) and (XXXVIII) are independently selected from —O—, C=0, NR⁶ or are absent;
R¹ and R² of Formula (XXXVII) and (XXXVIII) are selected from optionally substituted alkyl, optionally substituted cycloalkyl, optionally substituted alkylaryl or optionally substituted aryl;
R³ and R⁴ of Formula (XXXVII) and (XXXVIII) are independently selected from optionally substituted alkyl, optionally substituted cycloalkyl, optionally substituted cycloalkylalkyl, optionally substituted arylalkyl or optionally substituted aryl;
R⁵ and R⁶ of Formula (XXXVII) and (XXXVIII) are independently selected from optionally substituted alkyl or optionally substituted cycloalkyl;
R⁷ and R⁸ of Formula (XXXVII) and (XXXVIII) are independently selected from hydrogen, optionally substituted alkyl or optionally substituted cycloalkyl, or preferably methyl;
R⁹ and R¹⁰ of

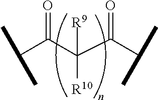

are independently selected from hydrogen, optionally substituted alkyl, or R⁹ and R¹⁰ may be taken together to form a ring;
R¹¹ to R¹⁴ of

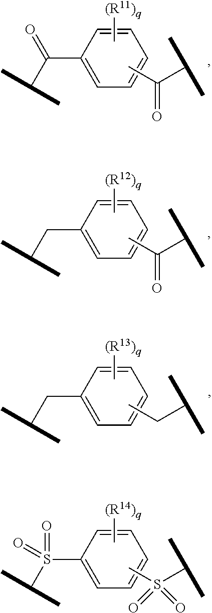

are independently selected from hydrogen, halogen, optionally substituted alkyl or OR¹⁵;
R¹⁵ of OR¹⁵ is selected from hydrogen, optionally substituted alkyl or optionally substituted cycloalkyl;
R¹⁶ and R¹⁷ of —(CR¹⁶R¹⁷)ₘ— are independently selected from hydrogen, halogen or optionally substituted alkyl;
R⁵⁰ and R⁵¹ of Formula (XXXVII) and (XXXVIII) are independently selected from optionally substituted alkyl, or R⁵⁰ and R⁵¹ are taken together to form a ring;
m and n of —(CR¹⁶R¹⁷)ₘ— and

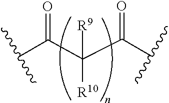

are independently an integer from 0-4;
o and p of

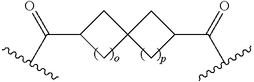

are independently an integer from 0-3;
q of

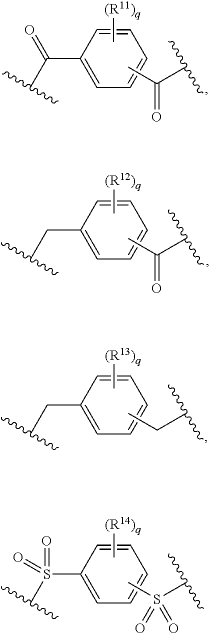

is an integer from 0-4; and
r of

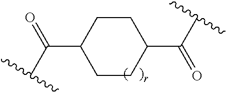

is an integer from 0-1;
or a pharmaceutically acceptable salt, tautomer or stereoisomer thereof.

In an embodiment, R¹ and R² of the ILM of Formula (XXXVII) or (XXXVIII) are t-butyl and R³ and R⁴ of the ILM of Formula (XXXVII) or (XXXVIII) are tetrahydronaphtalene.

In any of the compounds described herein, the ILM can have the structure of Formula (XXXIX) or (XL), which are derived from the IAP ligands described in WO Pub. No. 2013/071039, or an unnatural mimetic thereof:

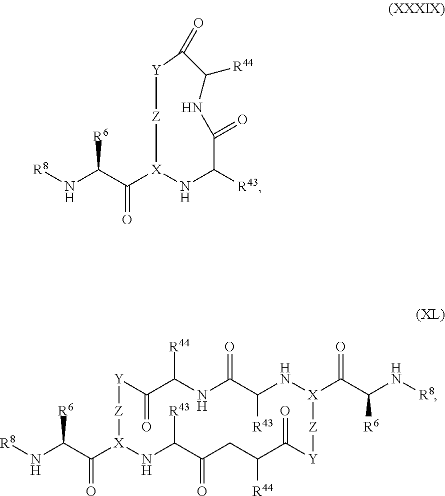

wherein:
R⁴³ and R⁴⁴ of Formulas (XXXIX) and (XL) are independently selected from hydrogen, alkyl, aryl, arylalkyl, heteroaryl, heteroarylalkyl, cycloalkyl, cycloalkylalkyl further optionally substituted, and
R⁶ and R⁸ of Formula (XXXIX) and (XL) are independently selected from hydrogen, optionally substituted alkyl or optionally substituted cycloalkyl.
each X of Formulas (XXXIX) and (XL) is independently selected from:

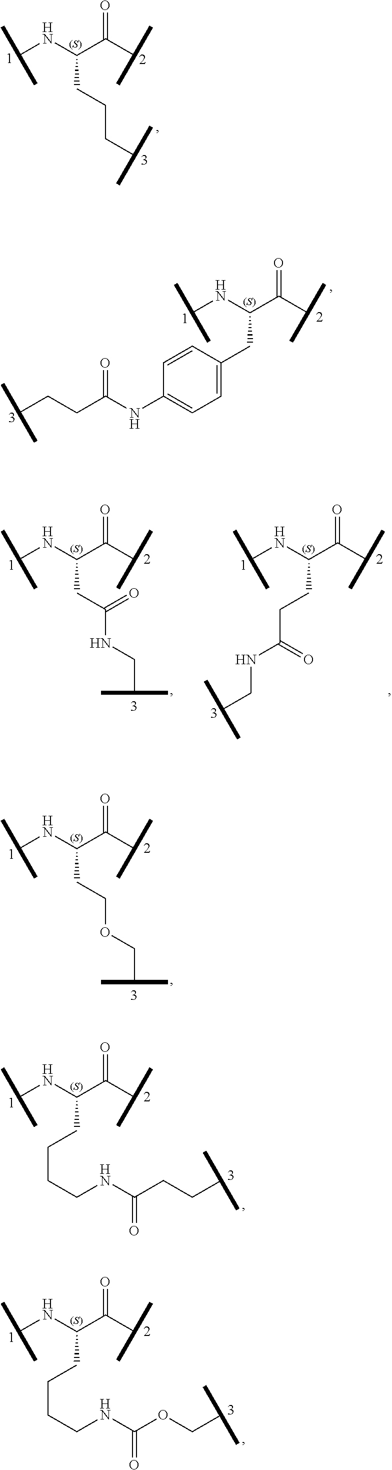

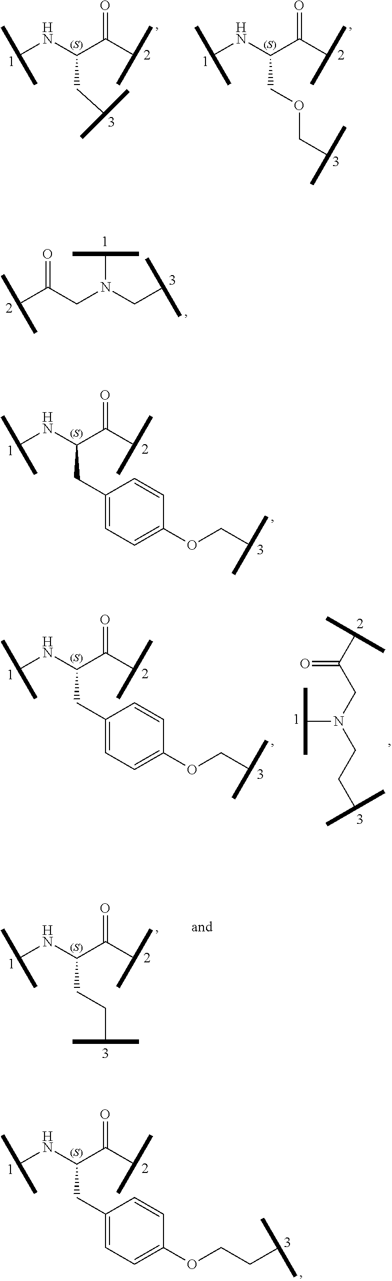

each Z of Formulas (XXXIX) and (XL) is selected from

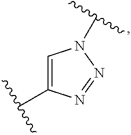 [SMILES: *c1cn(*)nn1]

wherein each

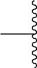

represents a point of attachment to the compound; and
each Y is selected from:

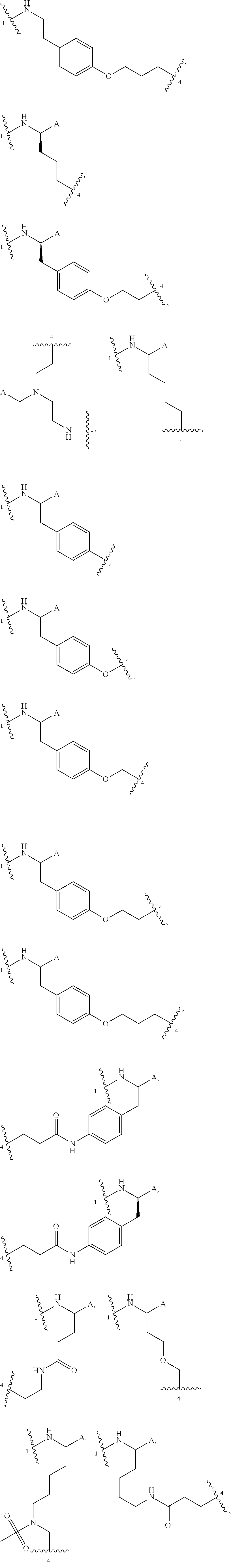

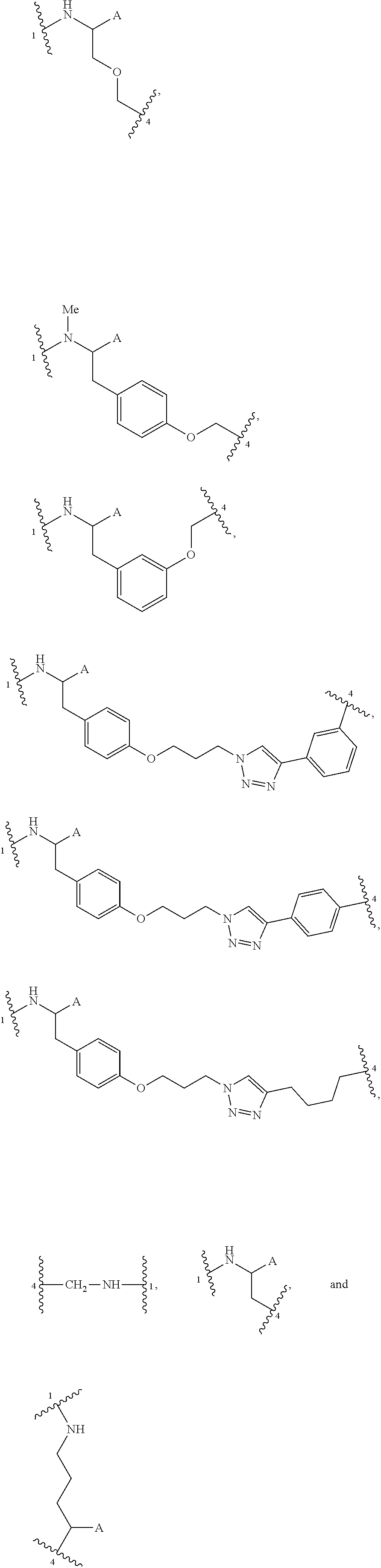

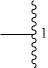

represents a point of attachment to a —C═O portion of the compound;

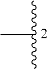

represents a point of attachment to an amino portion of the compound;

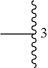

represents a first point of attachment to Z;

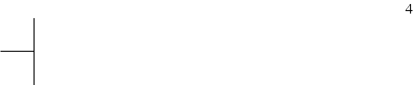

represents a second point of attachment to Z; and
A is selected from —C(O)R³ or

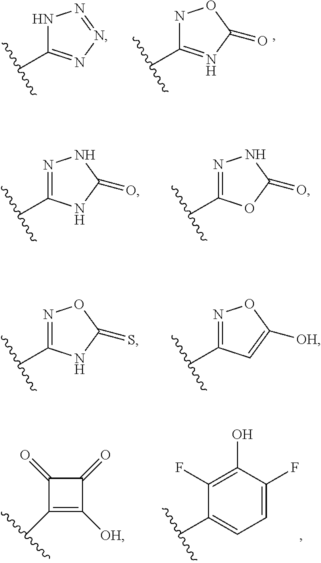

or a tautomeric form of any of the foregoing, wherein:
R³ of —C(O)R³ is selected from OH, NHCN, NHSO₂R¹⁰, NHOR¹¹ or N(R¹²)(R¹³);
R¹⁰ and R¹¹ of NHSO₂R¹⁰ and NHOR¹¹ are independently selected from —C₁-C₄ alkyl, cycloalkyl, aryl, heteroaryl, or heterocycloalkyl, any of which are optionally substituted, and hydrogen;
each of R¹² and R¹³ of N(R¹²)(R¹³) are independently selected from hydrogen, —C₁-C₄ alkyl, —(C₁-C₄ alkylene)-NH—(C₁-C₄ alkyl), benzyl, —(C₁-C₄ alkylene)-C(O)OH,
—(C₁-C₄alkylene)-C(O)CH₃, —CH(benzyl)-COOH, —C₁-C₄ alkoxy, and
—(C₁-C₄ alkylene)-O—(C₁-C₄ hydroxyalkyl); or R¹² and R¹³ of N(R¹²)(R¹³) are taken together with the nitrogen atom to which they are commonly bound to form a saturated heterocyclyl optionally comprising one additional heteroatom selected from N, O and S, and wherein the saturated heterocycle is optionally substituted with methyl.

In any of the compounds described herein, the ILM can have the structure of Formula (XLI), which are derived from the IAP ligands described in WO Pub. No. 2013/071039, or an unnatural mimetic thereof:

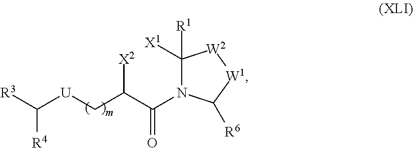

wherein:
W¹ of Formula (XLI) is selected from O, S, N—R^{A}, or C(R^{8a})(R^{8b});
W² of Formula (XLI) is selected from O, S, N—R^{A}, or C(R^{8c})(R^{8d}); provided that W¹ and W² are not both O, or both S;
R¹ of Formula (XLI) is selected from H, C₁-C₆alkyl, C₃-C₆cycloalkyl, —C₁-C₆alkyl-(substituted or unsubstituted C₃-C₆cycloalkyl), substituted or unsubstituted aryl, substituted or unsubstituted heteroaryl, —C₁-C₆alkyl-(substituted or unsubstituted aryl), or —C₁-C₆alkyl-(substituted or unsubstituted heteroaryl);
when X¹ is selected from O, N—R^{A}, S, S(O), or S(O)₂, then X² is C(R^{2a}R^{2b});
or:
X¹ of Formula (XLI) is selected from CR^{2c}R^{2d} and X² is CR^{2a}R^{2b} and R^{2c} and R^{2a} together form a bond;
or:
X¹ and X² of Formula (XLI) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring;
or:
X¹ of Formula (XLI) is selected from CH₂ and X² is C=0, C═C(R^{C})₂, or C═NR^{C}; where each R^{c} is independently selected from H, —CN, —OH, alkoxy, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);
R^{A} of N—R^{A} is selected from H, C₁-C₆alkyl, —C(═O)C₁-C₂alkyl, substituted or unsubstituted aryl, or substituted or unsubstituted heteroaryl;

R^{2a}, R^{2b}, R^{2c}, R^{2d} of CR^{2a}CR^{2d} and CR^{2a}R^{2b} are independently selected from H, substituted or unsubstituted C1-C6alkyl, substituted or unsubstituted C₁-C₆heteroalkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl) and —C(═O)R^{B};
R^{B} of —C(═O)R^{B} is selected from substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl), or —NR^{D}R^{E};
R^{D} and R^{E} of NR^{D}R^{E} are independently selected from H, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);
m of Formula (XLI) is selected from 0, 1 or 2;
—U— of Formula (XLI) is selected from —NHC(═O)—, —C(═O)NH—, —NHS(═O)₂—, —S(═O)₂NH—, —NHC(═O)NH—, —NH(C═O)O—, —O(C═O)NH—, or —NHS(═O)₂NH—;
R³ of Formula (XLI) is selected from C₁-C₃alkyl, or C₁-C₃fluoroalkyl;
R⁴ of Formula (XLI) is selected from —NHR⁵, —N(R⁵)2, —N+(R⁵)3 or —OR⁵;
each R⁵ of —NHR⁵, —N(R⁵)2, —N+(R⁵)3 and —OR⁵ is independently selected from H, C₁-C₃alkyl, C₁-C₃haloalkyl, C₁-C₃heteroalkyl and —C₁-C₃alkyl-(C₃-C₅cycloalkyl);
or:
R³ and R⁵ of Formula (XLI) together with the atoms to which they are attached form a substituted or unsubstituted 5-7 membered ring;
or:
R³ of Formula (XLI) is bonded to a nitrogen atom of U to form a substituted or unsubstituted 5-7 membered ring;
R⁶ of Formula (XLI) is selected from —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)2R⁷, —(C₁-C₃alkyl)-S(═O)2NHR⁷; —(C₁-C₃alkyl)-NHC(═O)NHR⁷, —(C₁-C₃alkyl)-NHS(═O)2NHR⁷, substituted or unsubstituted C₂-C₁₀heterocycloalkyl, or substituted or unsubstituted heteroaryl;
each R⁷ of —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)2R⁷, —(C₁-C₃alkyl)-S(═O)2NHR⁷; —(C₁-C₃alkyl)-NHC(═O)NHR⁷, —(C₁-C₃alkyl)-NHS(═O)2NHR⁷ is independently selected from C₁-C₆alkyl, C₁-C₆haloalkyl, C₁-C₆heteroalkyl, a substituted or unsubstituted C3-C10cycloalkyl, a substituted or unsubstituted C₂-C₁₀heterocycloalkyl, a substituted or unsubstituted aryl, a substituted or unsubstituted heteroaryl, —C₁-C₆alkyl-(substituted or unsubstituted C₃-C₁₀cycloalkyl), —C₁-C₆alkyl-(substituted or unsubstituted C2-C10heterocycloalkyl, —C1-C6alkyl-(substituted or unsubstituted aryl), —C₁-C₆alkyl-(substituted or unsubstituted heteroaryl), —(CH2)p-CH(substituted or unsubstituted aryl)2, —(CH₂)ₚ—CH(substituted or unsubstituted heteroaryl)2, —(CH₂)ₚ—CH(substituted or unsubstituted aryl)(substituted or unsubstituted heteroaryl), -(substituted or unsubstituted aryl)-(substituted or unsubstituted aryl), -(substituted or unsubstituted aryl)-(substituted or unsubstituted heteroaryl), -(substituted or unsubstituted heteroaryl)-(substituted or unsubstituted aryl), or -(substituted or unsubstituted heteroaryl)-(substituted or unsubstituted heteroaryl);
p of R⁷ is selected from 0, 1 or 2;
R^{8a}, R^{8b}, R^{8c}, and R^{8d} of C(R^{8a})(R^{8b}) and C(R^{8c})(R^{8d}) are independently selected from H, C₁-C₆alkyl, C₁-C₆fluoroalkyl, C₁-C₆ alkoxy, C₁-C₆heteroalkyl, and substituted or unsubstituted aryl;
or:
R^{8a} and R^{8d} are as defined above, and R^{8b} and R^{8c} together form a bond;
or:
R^{8a} and R^{8d} are as defined above, and R^{8b} and R^{8c} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;

or:
R^{8c} and R^{8d} are as defined above, and R^{8a} and R^{8b} together with the atoms to which they are attached form a substituted or unsubstituted saturated, or partially saturated 3-7 membered spirocycle or heterospirocycle comprising 1-3 heteroatoms selected from S, O and N;
or:
R^{8a} and R^{8b} are as defined above, and R^{8c} and R^{8d} together with the atoms to which they are attached form a substituted or unsubstituted saturated, or partially saturated 3-7 membered spirocycle or heterospirocycle comprising 1-3 heteroatoms selected from S, O and N;
where each substituted alkyl, heteroalkyl, fused ring, spirocycle, heterospirocycle, cycloalkyl, heterocycloalkyl, aryl or heteroaryl is substituted with 1-3 R⁹; and
each R⁹ of R^{8a}, R^{8b}, R^{8c} and R^{8d} is independently selected from halogen, —OH, —SH, (C═O), CN, C₁-C₄alkyl, C1-C4fluoroalkyl, C₁-C₄ alkoxy, C₁-C₄ fluoroalkoxy, —NH₂, —NH(C₁-C₄alkyl), —NH(C₁-C₄alkyl)₂, —C(═O)OH, —C(═O)NH₂, —C(═O)C₁-C₃alkyl, —S(═O)₂CH₃, —NH(C₁-C₄alkyl)-OH, —NH(C₁-C₄alkyl)-O—(C—C₄alkyl), —O(C₁-C₄alkyl)-NH2; —O(C₁-C₄alkyl)-NH—(C₁-C₄alkyl), and —O(C₁-C₄alkyl)-N—(C₁-C₄alkyl)₂, or two R⁹ together with the atoms to which they are attached form a methylene dioxy or ethylene dioxy ring substituted or unsubstituted with halogen, —OH, or C₁-C₃alkyl.

In any of the compounds described herein, the ILM can have the structure of Formula (XLII), which are derived from the IAP ligands described in WO Pub. No. 2013/071039, or an unnatural mimetic thereof:

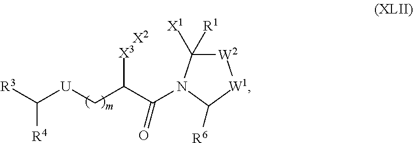

wherein:
W¹ of Formula (XLII) is O, S, N—R^{A}, or C(R^{8a})(R^{8b});
W² of Formula (XLII) is O, S, N—R^{A}, or C(R^{8c})(R^{8d}); provided that W¹ and W² are not both O, or both S;
R¹ of Formula (XLII) is selected from H, C₁-C₆alkyl, C₃-C₆cycloalkyl, —C₁-C₆alkyl-(substituted or unsubstituted C₃-C₆cycloalkyl), substituted or unsubstituted aryl, substituted or unsubstituted heteroaryl, —C₁-C₆alkyl-(substituted or unsubstituted aryl), or —C₁-C₆alkyl-(substituted or unsubstituted heteroaryl);
when X¹ of Formula (XLII) is N—R^{A}, then X² is C═O, or CR^{2c}R^{2d}, and X³ is CR^{2a}R^{2b};
or:
when X¹ of Formula (XLII) is selected from S, S(O), or S(O)₂, then X² is CR^{2c}R^{2d}, and X³ is CR^{2a}R^{2b};
or:
when X¹ of Formula (XLII) is O, then X² is CR^{2c}R^{2d} and N—R^{A} and X³ is CR^{2a}R^{2b};
or:
when X¹ of Formula (XLII) is CH₃, then X² is selected from O, N—R^{A}, S, S(O), or S(O)₂, and X³ is CR^{2a}R^{2b};
when X¹ of Formula (XLII) is CR^{2e}R^{2f} and X2 is CR^{2c}R^{2d}, and R^{2e} and R^{2c} together form a bond, and X³ of Formula (VLII) is CR^{2a}R^{2b};
or:
X¹ and X³ of Formula (XLII) are both CH₂ and X² of Formula (XLII) is C═O, C═C(R^{C})2, or C═NR^{c}; where each R^{c} is independently selected from H, —CN, —OH, alkoxy, substituted or unsubstituted C1-C6alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);
or:
X¹ and X² of Formula (XLII) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X³ is CR^{2a}R^{2b};
or:
X² and X³ of Formula (XLII) are independently selected from C and N, and are embers of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X¹ of Formula (VLII) is CR^{2e}R^{2f};
R^{A} of N—R^{A} is selected from H, C₁-C₆alkyl, —C(═O)C₁-C₂alkyl, substituted or unsubstituted aryl, or substituted or unsubstituted heteroaryl;

R^{2a}, R^{2b}, R^{2c}, R^{2d}, R^{2e}, and R^{2f} of CR^{2c}R^{2d}, R^{2a}R^{2b} and CR^{2e}R^{2f} are independently selected from H, substituted or unsubstituted C1-C6alkyl, substituted or unsubstituted C₁-C₆heteroalkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl) and —C(═O)R^{B};
R^{B} of —C(═O)R^{B} is selected from substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl), or —NR^{D}R^{E};
R^{D} and R^{E} of NR^{D}R^{E} are independently selected from H, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);
m of Formula (XLII) is selected from 0, 1 or 2;
—U— of Formula (XLII) is selected from —NHC(═O)—, —C(═O)NH—, —NHS(═O)₂—, —S(═O)₂NH—, —NHC(═O)NH—, —NH(C═O)O—, —O(C═O)NH—, or —NHS(═O)₂NH—;
R³ of Formula (XLII) is selected from C₁-C₃alkyl, or C₁-C₃fluoroalkyl;
R⁴ of Formula (XLII) is selected from —NHR⁵, —N(R⁵)₂, —N+(R⁵)₃ or —OR⁵;
each R⁵ of —NHR⁵, —N(R⁵)₂, —N+(R⁵)₃ and —OR⁵ is independently selected from H, C₁-C₃alkyl, C₁-C₃haloalkyl, C₁-C₃heteroalkyl and —C₁-C₃alkyl-(C₃-C₅cycloalkyl);
or:
R³ and R⁵ of Formula (XLII) together with the atoms to which they are attached form a substituted or unsubstituted 5-7 membered ring;
or:
R³ of Formula (XLII) is bonded to a nitrogen atom of U to form a substituted or unsubstituted 5-7 membered ring;
R⁶ of Formula (XLII) is selected from —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)₂R⁷, —(C₁-C₃alkyl)-S(═O)₂NHR⁷; —(C₁-C₃alkyl)-NHC(═O)NHR⁷, —(C₁-C₃alkyl)-NHS(═O)₂NHR⁷, substituted or unsubstituted C₂-C₁₀heterocycloalkyl, or substituted or unsubstituted heteroaryl;
each R⁷ of —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)₂R⁷, —(C₁-C₃alkyl)-S(═O)2NHR⁷; —(C₁-C₃alkyl)-NHC(═O)NHR⁷, —(C₁-C₃alkyl)-NHS(═O)2NHR⁷ is independently selected from C₁-C₆alkyl, C₁-C₆haloalkyl, C₁-C₆heteroalkyl, a substituted or unsubstituted C3-C10cycloalkyl, a substituted or unsubstituted C₂-C₁₀heterocycloalkyl, a substituted or unsubstituted aryl, a substituted or unsubstituted heteroaryl, —C₁-C₆alkyl-(substituted or unsubstituted C₃-C₁₀cycloalkyl), —C₁-C₆alkyl-(substituted or unsubstituted C2-C10heterocycloalkyl, —C1-C6alkyl-(substituted or unsubstituted aryl), —C₁-C₆alkyl-(substituted or unsubstituted heteroaryl), —(CH2)p-CH(substituted or unsubstituted aryl)2, —(CH₂)ₚ—CH(substituted or unsubstituted heteroaryl)2, —(CH₂)ₚ—CH(substituted or unsubstituted aryl)(substituted or unsubstituted heteroaryl), -(substituted or unsubstituted aryl)-(substituted or unsubstituted aryl), -(substituted or unsubstituted aryl)-(substituted or unsubstituted heteroaryl), -(substituted or unsubstituted heteroaryl)-(substituted or unsubstituted aryl), or -(substituted or unsubstituted heteroaryl)-(substituted or unsubstituted heteroaryl);
p of R⁷ is selected from 0, 1 or 2;
R^{8a}, R^{8b}, R^{8c}, and R^{8d} of C(R^{8a})(R^{8b}) and C(R^{8c})(R^{8d}) are independently selected from H, C₁-C₆alkyl, C₁-C₆fluoroalkyl, C₁-C₆ alkoxy, C₁-C₆heteroalkyl, and substituted or unsubstituted aryl;
or:
R^{8a} and R^{8d} are as defined above, and R^{8b} and R^{8c} together form a bond;
or:
R^{8a} and R^{8d} are as defined above, and R^{8b} and R^{8c} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;
or:
R^{8c} and R^{8d} are as defined above, and R^{8a} and R^{8b} together with the atoms to which they are attached form a substituted or unsubstituted saturated, or partially saturated 3-7 membered spirocycle or heterospirocycle comprising 1-3 heteroatoms selected from S, O and N;
or:
R^{8a} and R^{8b} are as defined above, and R^{8c} and R^{8d} together with the atoms to which they are attached form a substituted or unsubstituted saturated, or partially saturated 3-7 membered spirocycle or heterospirocycle comprising 1-3 heteroatoms selected from S, O and N;
where each substituted alkyl, heteroalkyl, fused ring, spirocycle, heterospirocycle, cycloalkyl, heterocycloalkyl, aryl or heteroaryl is substituted with 1-3 R⁹; and
each R⁹ of R^{8a}, R^{8b}, R^{8c} and R^{8d} is independently selected from halogen, —OH, —SH, (C═O), CN, C₁-C₄alkyl, C1-C4fluoroalkyl, C₁-C₄ alkoxy, C₁-C₄ fluoroalkoxy, —NH₂, —NH(C₁-C₄alkyl), —NH(C₁-C₄alkyl)₂, —C(═O)OH, —C(═O)NH₂, —C(═O)C₁-C₃alkyl, —S(═O)₂CH₃, —NH(C₁-C₄alkyl)-OH, —NH(C₁-C₄alkyl)-O—(C—C₄alkyl), —O(C₁-C₄alkyl)-NH2; —O(C₁-C₄alkyl)-NH—(C₁-C₄alkyl), and —O(C₁-C₄alkyl)-N—(C₁-C₄alkyl)₂, or two R⁹ together with the atoms to which they are attached form a methylene dioxy or ethylene dioxy ring substituted or unsubstituted with halogen, —OH, or C₁-C₃alkyl.

In any of the compounds described herein, the ILM can have the structure of Formula (XLIII), which is derived from the IAP ligands described in WO Pub. No. 2013/071039, or an unnatural mimetic thereof:

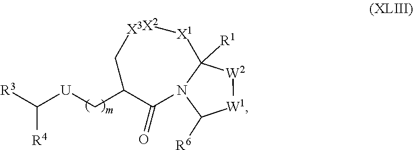

wherein:
W¹ of Formula (XLIII) is selected from O, S, N—R^{A}, or C(R^{8a})(R^{8b});
W² of Formula (XLIII) is selected from O, S, N—R^{A}, or C(R^{8c})(R^{8d}); provided that W¹ and W² are not both O, or both S;
R¹ of Formula (XLIII) is selected from H, C₁-C₆alkyl, C₃-C₆cycloalkyl, —C₁-C₆alkyl-(substituted or unsubstituted C₃-C₆cycloalkyl), substituted or unsubstituted aryl, substituted or unsubstituted heteroaryl, —C₁-C₆alkyl-(substituted or unsubstituted aryl), or —C₁-C₆alkyl-(substituted or unsubstituted heteroaryl);
when X¹ of Formula (XLIII) is selected from N—R^{A}, S, S(O), or S(O)₂, then X² of Formula (XLIII) is CR^{2c}R^{2d}; and X³ of Formula (XLIII) is CR^{2a}R^{2b};
or:
when X¹ of Formula (XLIII) is O, then X² of Formula (XLIII) is selected from O, N—R^{A}, S, S(O), or S(O)₂, and X³ of Formula (XLIII) is CR^{2a}R^{2b};
or:
when X¹ of Formula (XLIII) is CR^{2e}R^{2f} and X² of Formula (XLIII) is CR^{2c}R^{2d}, and R^{2e} and R^{2c} together form a bond, and X³ of Formula (XLIII) is CR^{2a}R^{2b};
or:
X¹ and X² of Formula (XLIII) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X³ of Formula (XLIII) is CR^{2a}R^{2b};
or:
X² and X³ of Formula (XLIII) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X¹ of Formula (VLII) is CR^{2e}R^{2f};
R^{A} of N—R^{A} is H, C₁-C₆alkyl, —C(═O)C₁-C₂alkyl, substituted or unsubstituted aryl, or substituted or unsubstituted heteroaryl;

R^{2a}, R^{2b}, R^{2c}, R^{2d}, R^{2e}, and R^{2f} of CR^{2c}CR^{2d}, CR^{2a}R^{2b} and CR^{2e}R^{2f} are independently selected from H, substituted or unsubstituted C₁-C₆alkyl, substituted or unsubstituted C₁-C₆heteroalkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl) and —C(═O)R^{B};
R^{B} of —C(═O)R^{B} is substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl), or —NR^{D}R^{E};
R^{D} and R^{E} of NR^{D}R^{E} are independently selected from H, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);
m of Formula (XLIII) is 0, 1 or 2;
—U— of Formula (XLIII) is —NHC(═O)—, —C(═O)NH—, —NHS(═O)₂—, —S(═O)₂NH—, —NHC(═O)NH—, —NH(C═O)O—, —O(C═O)NH—, or —NHS(═O)₂NH—;
R³ of Formula (XLIII) is C₁-C₃alkyl, or C₁-C₃fluoroalkyl;
R⁴ of Formula (XLIII) is —NHR⁵, —N(R⁵)₂, —N+(R⁵)₃ or —OR⁵;
each R⁵ of —NHR⁵, —N(R⁵)₂, —N+(R⁵)₃ and —OR⁵ is independently selected from H, C₁-C₃alkyl, C₁-C₃haloalkyl, C₁-C₃heteroalkyl and —C₁-C₃alkyl-(C₃-C₅cycloalkyl);
or:
R³ and R⁵ of Formula (XLIII) together with the atoms to which they are attached form a substituted or unsubstituted 5-7 membered ring;
or:
R³ of Formula (XLIII) is bonded to a nitrogen atom of U to form a substituted or unsubstituted 5-7 membered ring;
R⁶ of Formula (XLIII) is selected from —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)₂R⁷, —(C₁-C₃alkyl)-S(═O)₂NHR⁷; —(C₁-C₃alkyl)-NHC(═O)NHR⁷, —(C₁-C₃alkyl)-NHS(═O)₂NHR⁷, substituted or unsubstituted C₂-C₁₀heterocycloalkyl, or substituted or unsubstituted heteroaryl;
each R⁷ of —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)2R⁷, —(C₁-C₃alkyl)-S(═O)2NHR⁷; —(C₁-C₃alkyl)-NHC(═O)NHR⁷, —(C₁-C₃alkyl)-NHS(═O)2NHR⁷ is independently selected from C₁-C₆alkyl, C₁-C₆haloalkyl, C₁-C₆heteroalkyl, a substituted or unsubstituted C3-C10cycloalkyl, a substituted or unsubstituted C₂-C₁₀heterocycloalkyl, a substituted or unsubstituted aryl, a substituted or unsubstituted heteroaryl, —C₁-C₆alkyl-(substituted or unsubstituted C₃-C₁₀cycloalkyl), —C₁-C₆alkyl-(substituted or unsubstituted C2-C10heterocycloalkyl, —C1-C6alkyl-(substituted or unsubstituted aryl), —C₁-C₆alkyl-(substituted or unsubstituted heteroaryl), —(CH2)p-CH(substituted or unsubstituted aryl)2, —(CH₂)ₚ—CH(substituted or unsubstituted heteroaryl)2, —(CH₂)ₚ—CH(substituted or unsubstituted aryl)(substituted or unsubstituted heteroaryl), -(substituted or unsubstituted aryl)-(substituted or unsubstituted aryl), -(substituted or unsubstituted aryl)-(substituted or unsubstituted heteroaryl), -(substituted or unsubstituted heteroaryl)-(substituted or unsubstituted aryl), or -(substituted or unsubstituted heteroaryl)-(substituted or unsubstituted heteroaryl);
p of R⁷ is 0, 1 or 2;
R^{8a}, R^{8b}, R^{8c}, and R^{8d} of C(R^{8a})(R^{8b}) and C(R^{8c})(R^{8d}) are independently selected from H, C₁-C₆alkyl, C₁-C₆fluoroalkyl, C₁-C₆ alkoxy, C₁-C₆heteroalkyl, and substituted or unsubstituted aryl;
or:
R^{8a} and R^{8d} are as defined above, and R^{8b} and R^{8c} together form a bond;
or:
R^{8a} and R^{8d} are as defined above, and R^{8b} and R^{8c} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;
or:
R^{8c} and R^{8d} are as defined above, and R^{8a} and R^{8b} together with the atoms to which they are attached form a substituted or unsubstituted saturated, or partially saturated 3-7 membered spirocycle or heterospirocycle comprising 1-3 heteroatoms selected from S, O and N;
or:
R^{8a} and R^{8b} are as defined above, and R^{8c} and R^{8d} together with the atoms to which they are attached form a substituted or unsubstituted saturated, or partially saturated 3-7 membered spirocycle or heterospirocycle comprising 1-3 heteroatoms selected from S, O and N;
where each substituted alkyl, heteroalkyl, fused ring, spirocycle, heterospirocycle, cycloalkyl, heterocycloalkyl, aryl or heteroaryl is substituted with 1-3 R⁹; and
each R⁹ of R^{8a}, R^{8b}, R^{8c} and R^{8d} is independently selected from halogen, —OH, —SH, (C═O), CN, C₁-C₄alkyl, C1-C4fluoroalkyl, C₁-C₄ alkoxy, C₁-C₄ fluoroalkoxy, —NH₂, —NH(C₁-C₄alkyl), —NH(C₁-C₄alkyl)₂, —C(═O)OH, —C(═O)NH₂, —C(═O)C₁-C₃alkyl, —S(═O)₂CH₃, —NH(C₁-C₄alkyl)-OH, —NH(C₁-C₄alkyl)-O—(C—C₄alkyl), —O(C₁-C₄alkyl)-NH2; —O(C₁-C₄alkyl)-NH—(C₁-C₄alkyl), and —O(C₁-C₄alkyl)-N—(C₁-C₄alkyl)₂, or two R⁹ together with the atoms to which they are attached form a methylene dioxy or ethylene dioxy ring substituted or unsubstituted with halogen, —OH, or C₁-C₃alkyl.

In any of the compounds described herein, the ILM can have the structure of Formula (XLIV), which is derived from the IAP ligands described in WO Pub. No. 2013/071039, or an unnatural mimetic thereof:

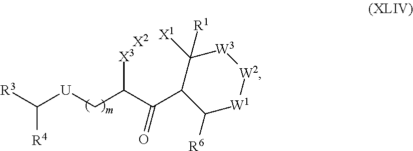

wherein:
W¹ of Formula (XLIV) is selected from O, S, N—R^{A}, or C(R^{8a})(R^{8b});
W² of Formula (XLIV) is selected from O, S, N—R^{A}, or C(R^{8c})(R^{8d}); provided that W¹ and W² are not both O, or both S;
W³ of Formula (XLIV) is selected from O, S, N—R^{A}, or C(R^{8e})(R^{8f}), providing that the ring comprising W¹, W², and W³ does not comprise two adjacent oxygen atoms or sulfur atoms;
R¹ of Formula (XLIV) is selected from H, C₁-C₆alkyl, C₃-C₆cycloalkyl, —C₁-C₆alkyl-(substituted or unsubstituted C₃-C₆cycloalkyl), substituted or unsubstituted aryl, substituted or unsubstituted heteroaryl, —C₁-C₆alkyl-(substituted or unsubstituted aryl), or C₁-C₆alkyl-(substituted or unsubstituted heteroaryl);
when X¹ of Formula (XLIV) is O, then X² of Formula (XLIV) is selected from CR^{2c}R^{2d} and N—R^{A}, and X³ of Formula (XLIV) is CR^{2a}R^{2b};
or:
when X¹ of Formula (XLIV) is CH₂, then X² of Formula (XLIV) is selected from O, N—R^{A}, S, S(O), or S(O)₂, and X³ of Formula (XLIV) is CR^{2a}R^{2b};
or:
when X¹ of Formula (XLIV) is CR^{2e}R^{2f} and X² of Formula (XLIV) is CR^{2c}R^{2d}, and R^{2e} and R^{2c} together form a bond, and X³ of Formula (VLIV) is CR^{2a}R^{2b};
or:
X¹ and X³ of Formula (XLIV) are both CH₂ and X² of Formula (XLII) is C=0, C═C(R^{C})2, or C═NR^{C}; where each R^{C} is independently selected from H, —CN, —OH, alkoxy, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₆heterocycloalkyl), —C₁-C₆alkyl-(substituted or unsubstituted aryl), or —C₁-C₆alkyl-(substituted or unsubstituted heteroaryl);
or:
X¹ and X² of Formula (XLIV) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X³ of Formula (XLIV) is CR^{2a}R^{2b};
or:
X² and X³ of Formula (XLIV) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X¹ of Formula (VLIV) is CR^{2e}R^{2f};
R^{A} of N—R^{A} is selected from H, C₁-C₆alkyl, —C(═O)C₁-C₂alkyl, substituted or unsubstituted aryl, or substituted or unsubstituted heteroaryl;
R^{2a}, R^{2b}, R^{2c}, R^{2d}, R^{2e}, and R^{2f} of CR^{2c}R^{2d}, CR^{2a}R^{2b} and CR^{2e}R^{2f} are independently selected from H, substituted or unsubstituted C₁-C₆alkyl, substituted or unsubstituted C₁-C₆heteroalkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl) and —C(═O)R^{B};
R^{B} of —C(═O)R^{B} is selected from substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl), or —NR^{D}R^{E};
R^{D} and R^{E} of NR^{D}R^{E} are independently selected from H, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);
m of Formula (XLIV) is selected from 0, 1 or 2;
—U— of Formula (XLIV) is selected from —NHC(═O)—, —C(═O)NH—, —NHS(═O)₂—, —S(═O)₂NH—, —NHC(═O)NH—, —NH(C═O)O—, —O(C═O)NH—, or —NHS(═O)₂NH—;
R³ of Formula (XLIV) is selected from C₁-C₃alkyl, or C₁-C₃fluoroalkyl;
R⁴ of Formula (XLIV) is selected from —NHR⁵, —N(R⁵)₂, —N+(R⁵)₃ or —OR⁵;
each R⁵ of —NHR⁵, —N(R⁵)₂, —N+(R⁵)₃ and —OR⁵ is independently selected from H, C₁-C₃alkyl, C₁-C₃haloalkyl, C₁-C₃heteroalkyl and —C₁-C₃alkyl-(C₃-C₅cycloalkyl);
or:
R³ and R⁵ of Formula (XLIV) together with the atoms to which they are attached form a substituted or unsubstituted 5-7 membered ring;
or:
R³ of Formula (XLIII) is bonded to a nitrogen atom of U to form a substituted or unsubstituted 5-7 membered ring;
R⁶ of Formula (XLIII) is selected from —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)₂R⁷, —(C₁-C₃alkyl)-S(═O)₂NHR⁷; —(C₁-C₃alkyl)-NHC(═O)NHR⁷, —(C₁-C₃alkyl)-NHS(═O)₂NHR⁷, substituted or unsubstituted C₂-C₁₀heterocycloalkyl, or substituted or unsubstituted heteroaryl;
each R⁷ of —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)2R⁷, —(C₁-C₃alkyl)-S(═O)2NHR⁷; —(C₁-C₃alkyl)-NHC(═O)NHR⁷, —(C₁-C₃alkyl)-NHS(═O)2NHR⁷ is independently selected from C₁-C₆alkyl, C₁-C₆haloalkyl, C₁-C₆heteroalkyl, a substituted or unsubstituted C3-C10cycloalkyl, a substituted or unsubstituted C₂-C₁₀heterocycloalkyl, a substituted or unsubstituted aryl, a substituted or unsubstituted heteroaryl, —C₁-C₆alkyl-(substituted or unsubstituted C₃-C₁₀cycloalkyl), —C₁-C₆alkyl-(substituted or unsubstituted C2-C10heterocycloalkyl, —C1-C6alkyl-(substituted or unsubstituted aryl), —C₁-C₆alkyl-(substituted or unsubstituted heteroaryl), —(CH2)p-CH(substituted or unsubstituted aryl)2, —(CH₂)ₚ—CH(substituted or unsubstituted heteroaryl)2, —(CH₂)ₚ—CH(substituted or unsubstituted aryl)(substituted or unsubstituted heteroaryl), -(substituted or unsubstituted aryl)-(substituted or unsubstituted aryl), -(substituted or unsubstituted aryl)-(substituted or unsubstituted heteroaryl), -(substituted or unsubstituted heteroaryl)-(substituted or unsubstituted aryl), or -(substituted or unsubstituted heteroaryl)-(substituted or unsubstituted heteroaryl);
p of R⁷ is selected from 0, 1 or 2;
R^{8a}, R^{8b}, R^{8c}, R^{8d}, R^{8e}, and R^{8f} of C(R^{8a})(R^{8b}), C(R^{8c})(R^{8d}) and C(R^{8e})(R^{8f}) are independently selected from H, C₁-C₆alkyl, C₁-C₆fluoroalkyl, C₁-C₆ alkoxy, C₁-C₆heteroalkyl, and substituted or unsubstituted aryl;

or:
R^{8a}, R^{8d}, R^{8e}, and R^{8f} of C(R^{8a})(R^{8b}), C(R^{8c})(R^{8d}) and C(R^{8e})(R^{8f}) are as defined above, and R^{8b} and R^{8c} together form a bond;
or:
R^{8a}, R^{8b}, R^{8d}, and R^{8f} of C(R^{8a})(R^{8b}), C(R^{8c})(R^{8d}) and C(R^{8e})(R^{8f}) are as defined above, and R^{8b} and R^{8c} together form a bond;
or:
R^{8a}, R^{8d}, R^{8e}, and R^{8f} of C(R^{8a})(R^{8b}), C(R^{8c})(R^{8d}) and C(R^{8e})(R^{8f}) are as defined above, and R^{8b} and R^{8c} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;
or:
R^{8a}, R^{8b}, R^{8d}, and R^{8f} of C(R^{8a})(R^{8b}), C(R^{8c})(R^{8d}) and C(R^{8e})(R^{8f}) are as defined above, and R^{8c} and R^{8e} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;
or:
R^{8c}, R^{8d}, R^{8e}, and R^{8f} of C(R^{8c})(R^{8d}) and C(R^{8e})(R^{8f}) are as defined above, and R^{8a} and R^{8b} together with the atoms to which they are attached form a substituted or unsubstituted saturated, or partially saturated 3-7 membered spirocycle or heterospirocycle comprising 1-3 heteroatoms selected from S, O and N;
or:
R^{8a}, R^{8b}, R^{8e}, and R^{8f} of C(R^{8a})(R^{8b}) and C(R^{8e})(R^{8f}) are as defined above, and R^{8c} and R^{8d} together with the atoms to which they are attached form a substituted or unsubstituted saturated, or partially saturated 3-7 membered spirocycle or heterospirocycle comprising 1-3 heteroatoms selected from S, O and N;
or:
R^{8a}, R^{8b}, R^{8e}, and R^{8d} of C(R^{8a})(R^{8b}) and C(R^{8c})(R^{8d}) are as defined above, and R^{8e} and R^{8f} together with the atoms to which they are attached form a substituted or unsubstituted saturated, or partially saturated 3-7 membered spirocycle or heterospirocycle comprising 1-3 heteroatoms selected from S, O and N;
or:
where each substituted alkyl, heteroalkyl, fused ring, spirocycle, heterospirocycle, cycloalkyl, heterocycloalkyl, aryl or heteroaryl is substituted with 1-3 R⁹; and
each R⁹ of R^{8a}, R^{8b}, R^{8c}, R^{8d}, R^{8e}, and R^{8f} is independently selected from halogen, —OH, —SH, (C═O), CN, C₁-C₄alkyl, C1-C4fluoroalkyl, C₁-C₄ alkoxy, C₁-C₄ fluoroalkoxy, —NH₂, —NH(C₁— C₄alkyl), —NH(C₁-C₄alkyl)₂, —C(═O)OH, —C(═O)NH₂, —C(═O)C₁-C₃alkyl, —S(═O)₂CH₃, —NH(C₁-C₄alkyl)-OH, —NH(C₁-C₄alkyl)-O—(C—C₄alkyl), —O(C₁-C₄alkyl)-NH2; —O(C₁-C₄alkyl)-NH—(C₁-C₄alkyl), and —O(C₁-C₄alkyl)-N—(C₁-C₄alkyl)₂, or two R⁹ together with the atoms to which they are attached form a methylene dioxy or ethylene dioxy ring substituted or unsubstituted with halogen, —OH, or C₁-C₃alkyl.

In any of the compounds described herein, the ILM can have the structure of Formula (XLV), (XLVI) or (XLVII), which is derived from the IAP ligands described in Vamos, M., et al., Expedient synthesis of highly potent antagonists of inhibitor of apoptosis proteins (IAPs) with unique selectivity for ML-IAP, ACS Chem. Biol., 8(4), 725-32 (2013), or an unnatural mimetic thereof:

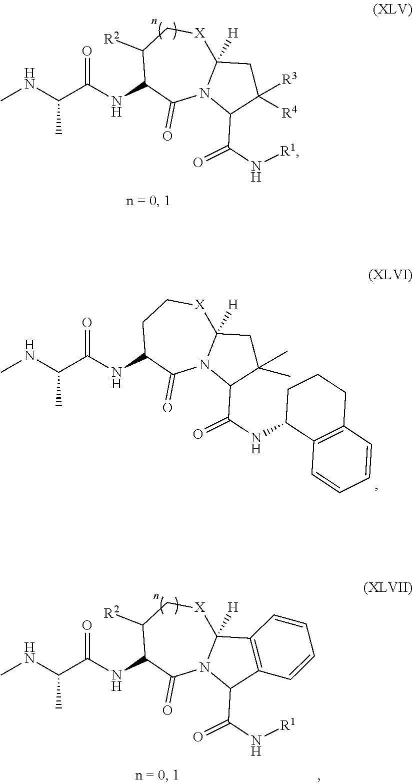

wherein:
R², R³ and R⁴ of Formula (XLV) are independently selected from H or ME;
X of Formula (XLV) is independently selected from O or S; and
R¹ of Formula (XLV) is selected from:

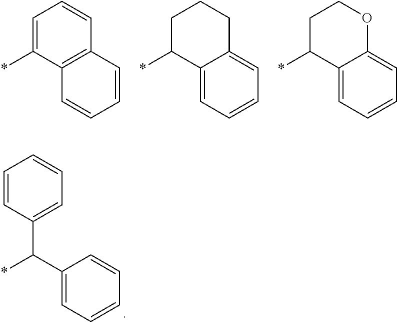

In a particular embodiment, the ILM has a structure according to Formula (XLVIII):

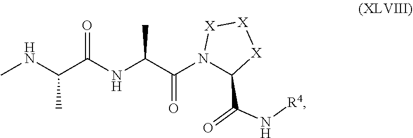

wherein R³ and R⁴ of Formula (XLVIII) are independently selected from H or ME;

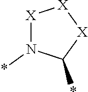

is a 5-member heteocycle selected from:

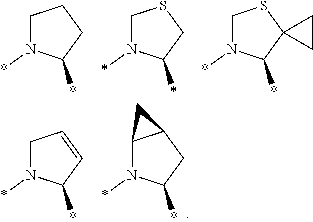

In a particular embodiment, the

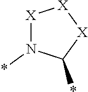

### of Formula XLVIII) is

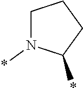 [SMILES: *[C@@H]1CCCN1*]

In a particular embodiment, the ILM has a structure and attached to a linker group L as shown below:

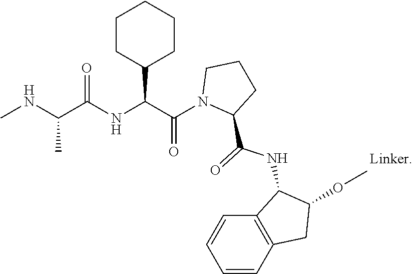

In a particular embodiment, the ILM has a structure according to Formula (XLIX), (L), or (LI):

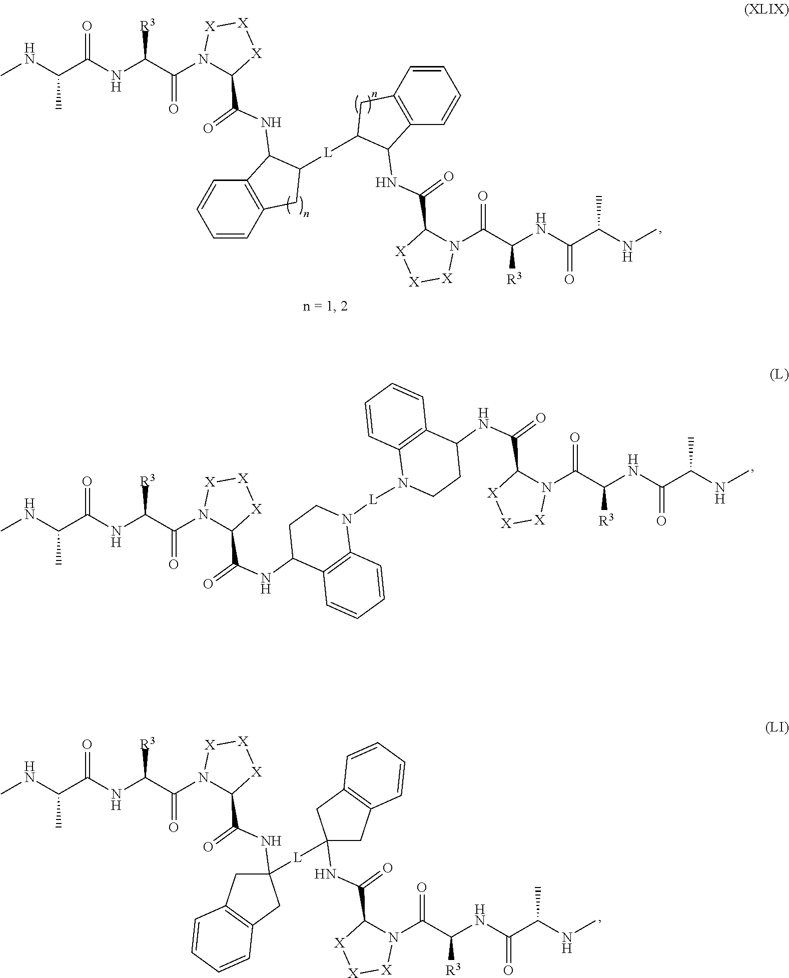

wherein:
R³ of Formula (XLIX), (L) or (LI) are independently selected from H or ME;

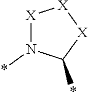

is a 5-member heteocycle selected from:

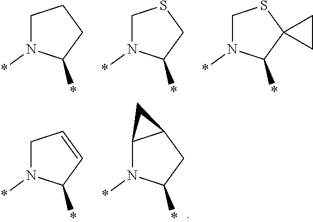

and L of Formula (XLIX), (L) or (LI) is selected from:

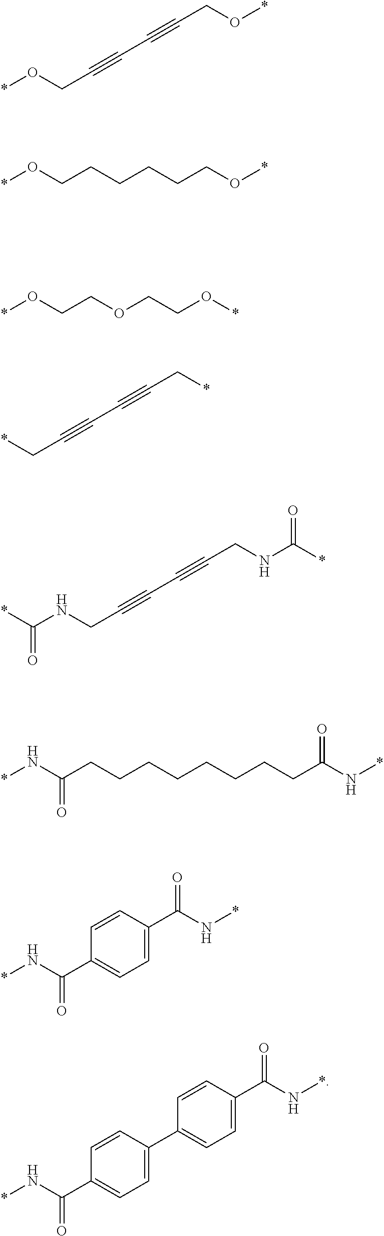

In a particular embodiment, L of Formula (XLIX), (L), or (LI)

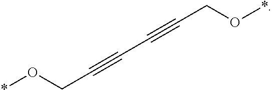

In a particular embodiment, the ILM has a structure according to Formula (LII):

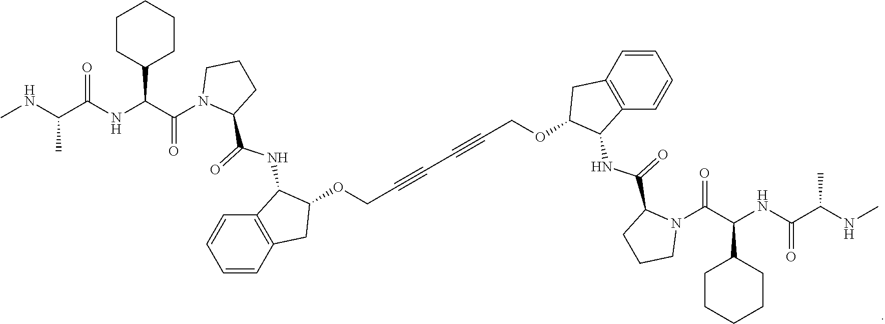

In a particular embodiment, the ILM according to Formula (LII) is chemically linked to the linker group L in the area denoted with

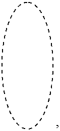

and as shown below:

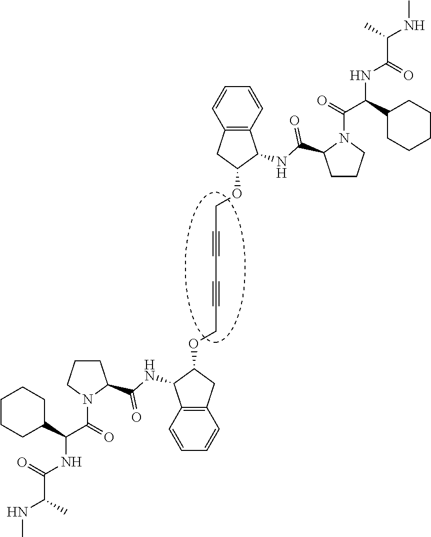 [SMILES: CN[C@@H](C)C(=O)N[C@H](C(=O)N1CCC[C@H]1C(=O)N[C@H]1c2ccccc2C[C@H]1OCC#CC#CCO[C@@H]1Cc2ccccc2[C@@H]1NC(=O)[C@@H]1CCCN1C(=O)[C@@H](NC(=O)[C@H](C)NC)C1CCCCC1)C1CCCCC1]

In any of the compounds described herein, the ILM can have the structure of Formula (LIII) or (LIV), which is based on the IAP ligands described in Hennessy, E J, et al., Discovery of aminopiperidine-based Smac mimetics as IAP antagonists, Bioorg. Med. Chem. Lett., 22(4), 1960-4 (2012), or an unnatural mimetic thereof:

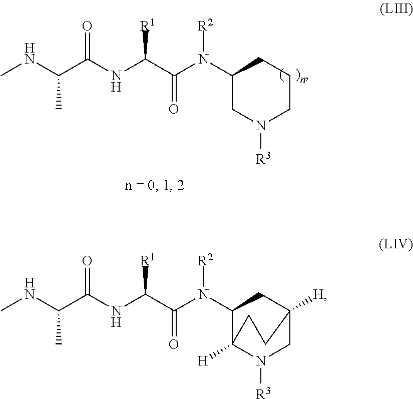

wherein:

R¹ of Formulas (LIII) and (LIV) is selected from:

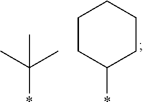

R² of Formulas (LIII) and (LIV) is selected from H or Me;

R³ of Formulas (LIII) and (LIV) is selected from:

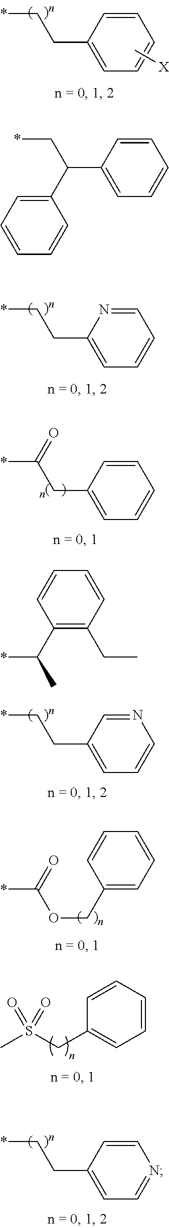

X of is selected from H, halogen, methyl, methoxy, hydroxy, nitro or trifluoromethyl.

In any of the compounds described herein, the ILM can have the structure of and be chemically linked to the linker as shown in Formula (LV) or (LVI), or an unnatural mimetic thereof:

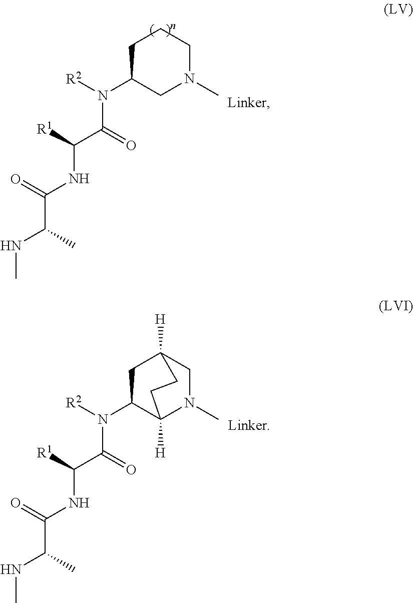

In any of the compounds described herein, the ILM can have the structure of Formula (LVII), which is based on the IAP ligands described in Cohen, F, et al., Orally bioavailable antagonists of inhibitor of apoptosis proteins based on an azabicyclooctane scaffold, J. Med. Chem., 52(6), 1723-30 (2009), or an unnatural mimetic thereof:

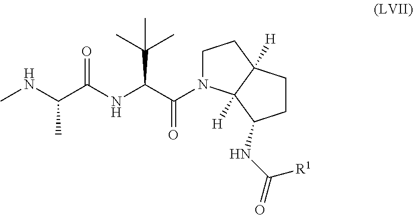

wherein:

R1 of Formulas (LVII) is selected from:

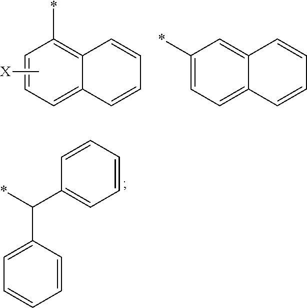

X of

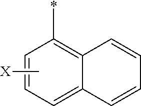

is selected from H, fluoro, methyl or methoxy.

In a particular embodiment, the ILM is represented by the following structure:

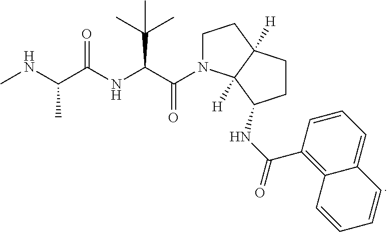 [SMILES: CN[C@@H](C)C(=O)N[C@H](C(=O)N1CC[C@H]2CC[C@H](NC(=O)c3cccc4ccccc34)[C@H]21)C(C)(C)C]

In a particular embodiment, the ILM is selected from the group consisting of, and which the chemical link between the ILM and linker group L is shown:

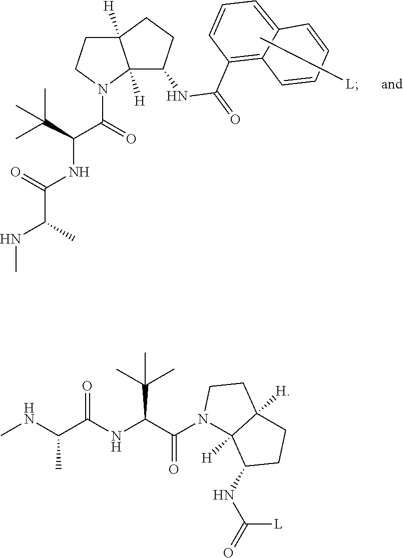

In any of the compounds described herein, the ILM is selected from the group consisting of the structures below, which are based on the IAP ligands described in Asano, M, et al., Design, sterioselective synthesis, and biological evaluation of novel tri-cyclic compounds as inhibitor of apoptosis proteins (IAP) antagonists, Bioorg. Med. Chem., 21(18): 5725-37 (2013), or an unnatural mimetic thereof:

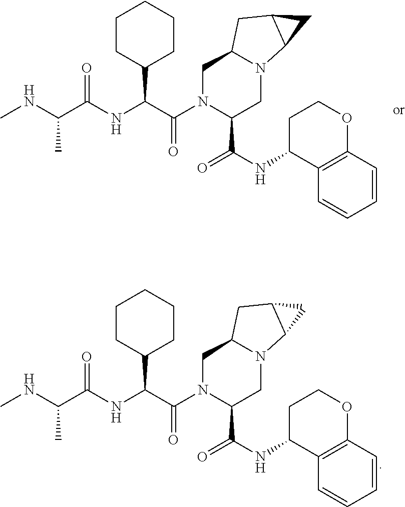 [SMILES: CN[C@@H](C)C(=O)N[C@H](C(=O)N1C[C@H]2C[C@H]3C[C@H]3N2C[C@H]1C(=O)N[C@@H]1CCOc2ccccc21)C1CCCCC1 ; CN[C@@H](C)C(=O)N[C@H](C(=O)N1C[C@H]2C[C@@H]3C[C@@H]3N2C[C@H]1C(=O)N[C@@H]1CCOc2ccccc21)C1CCCCC1]

In a particular embodiment, the ILM is selected from the group consisting of, and which the chemical link between the ILM and linker group L is shown:

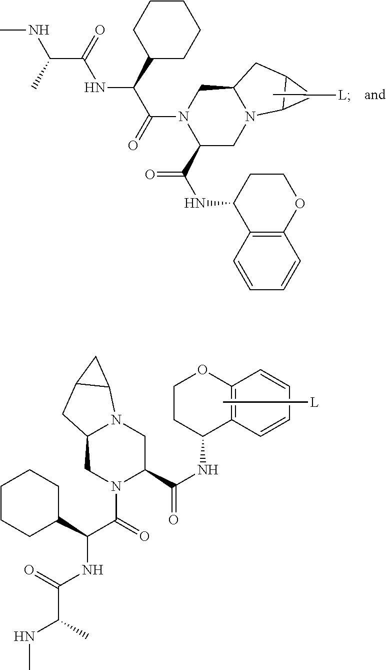

In any of the compounds described herein, the ILM can have the structure of Formula (LVIII), which is based on the IAP ligands described in Asano, M, et al., Design, sterioselective synthesis, and biological evaluation of novel tri-cyclic compounds as inhibitor of apoptosis proteins (IAP) antagonists, Bioorg. Med. Chem., 21(18): 5725-37 (2013), or an unnatural mimetic thereof:

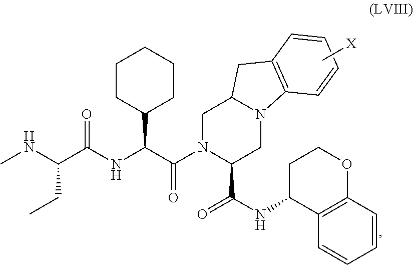

wherein X of Formula (LVIII) is one or two substituents independently selected from H, halogen or cyano.

In any of the compounds described herein, the ILM can have the structure of and be chemically linked to the linker group L as shown in Formula (LIX) or (LX), or an unnatural mimetic thereof:

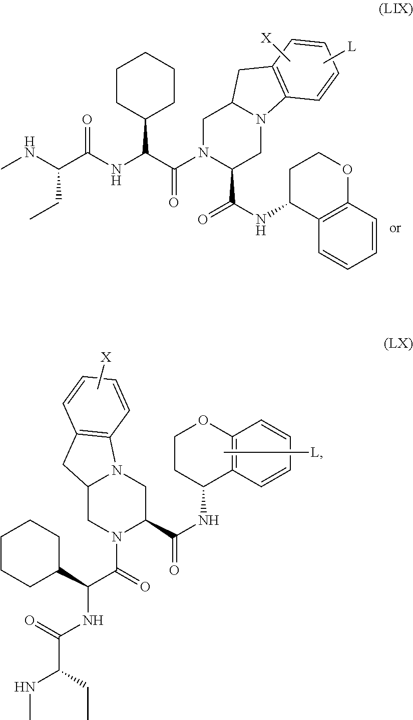

wherein X of Formula (LIX) and (LX) is one or two substituents independently selected from H, halogen or cyano, and; and L of Formulas (LIX) and (LX) is a linker group as described herein.

In any of the compounds described herein, the ILM can have the structure of Formula (LXI), which is based on the IAP ligands described in Ardecky, R J, et al., Design, synthesis and evaluation of inhibitor of apoptosis (IAP) antagonists that are highly selective for the BIR2 domain of XIAP, Bioorg. Med. Chem., 23(14): 4253-7 (2013), or an unnatural mimetic thereof:

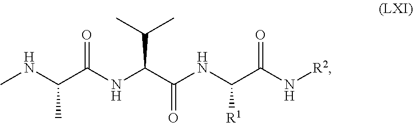

wherein:

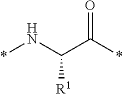

of Formula (LXI) is a natural or unnatural amino acid; and R² of Formula (LXI) is selected from:

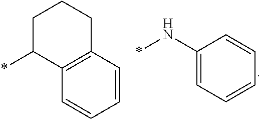

In any of the compounds described herein, the ILM can have the structure of and be chemically linked to the linker group L as shown in Formula (LXII) or (LLXIII), or an unnatural mimetic thereof:

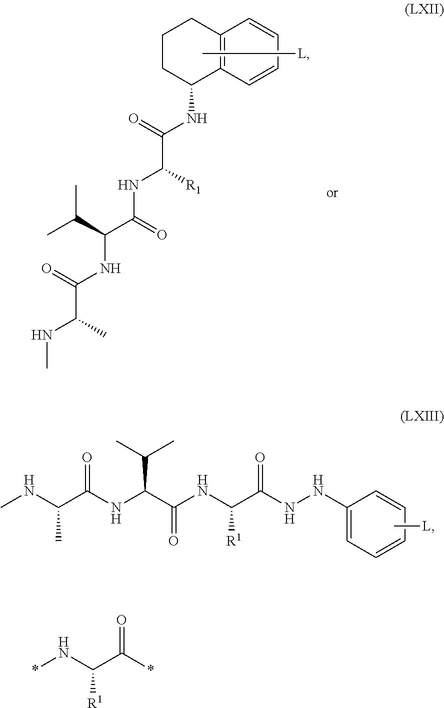

of Formula (LXI) is a natural or unnatural amino acid; and
L of Formula (LXI) is a linker group as described herein.

In any of the compounds described herein, the ILM can have the structure selected from the group consisting of, which is based on the IAP ligands described in Wang, J, et al., Discovery of novel second mitochondrial-derived activator of caspase mimetics as selective inhibitor or apoptosis protein inhibitors, J. Pharmacol. Exp. Ther., 349(2): 319-29 (2014), or an unnatural mimetic thereof:

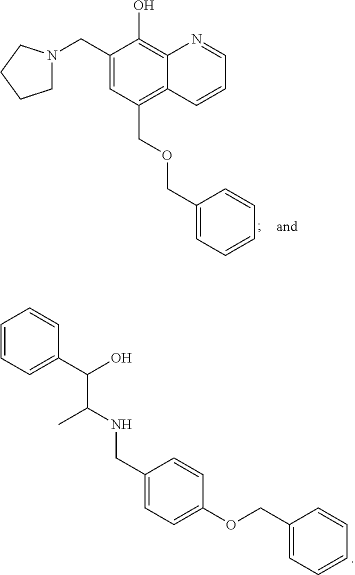 [SMILES: Oc1c(CN2CCCC2)cc(COCc2ccccc2)c2cccnc12 ; CC(NCc1ccc(OCc2ccccc2)cc1)C(O)c1ccccc1]

In any of the compounds described herein, the ILM has a structure according to Formula (LXIX), which is based on the IAP ligands described in Hird, A W, et al., Structure-based design and synthesis of tricyclic IAP (Inhibitors of Apoptosis Proteins) inhibitors, Bioorg. Med. Chem. Lett., 24(7): 1820-4 (2014), or an unnatural mimetic thereof:

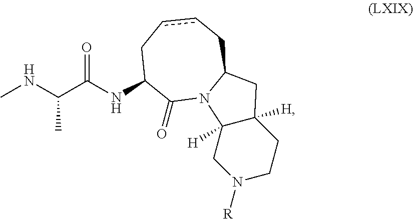

wherein R of Formula LIX is selected from the group consisting of:

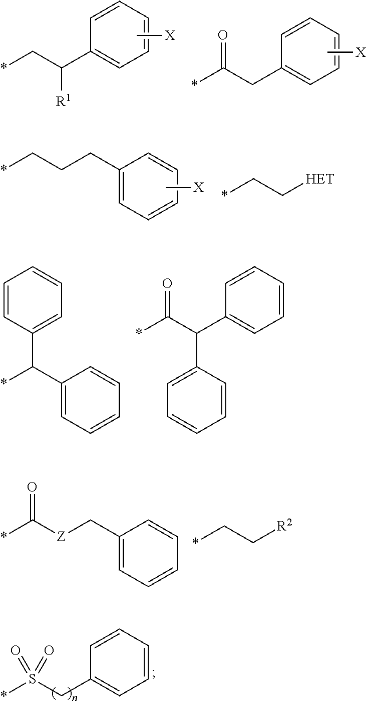

### R1 of

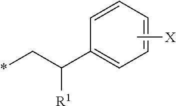

is selected from H or Me;

### R2 of

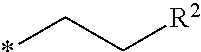

is selected from alkyl or cycloalkyl;

### X of

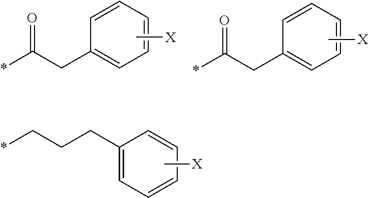

is 1-2 substitutents independently selected from halogen, hydroxy, methoxy, nitro and trifluoromethyl

### Z of

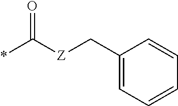

### is O or NH;

### HET of

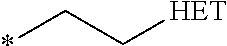

is mono- or fused bicyclic heteroaryl; and
- - - of Formula (LIX) is an optional double bond.

In a particular embodiment, the ILM of the compound has a chemical structure as represented by:

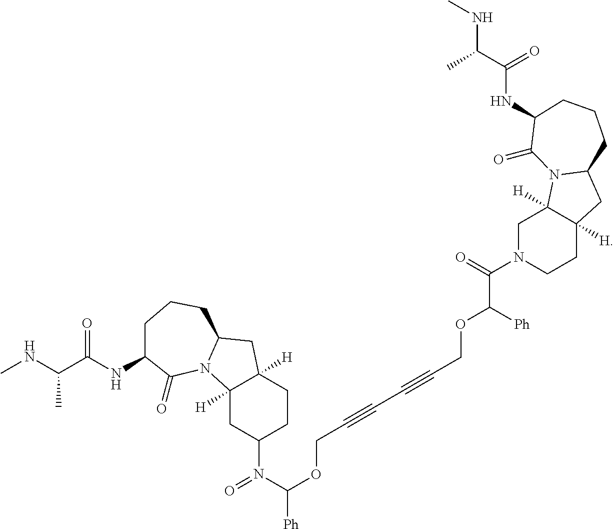

In a particular embodiment, the ILM of the compound has a chemical structure selected from the group consisting of:

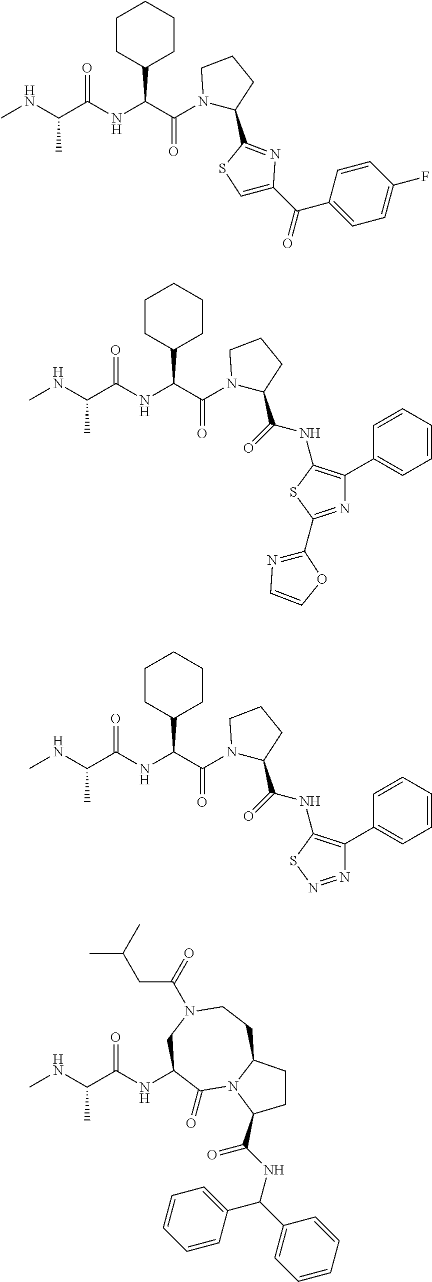 [SMILES: CN[C@@H](C)C(=O)N[C@H](C(=O)N1CCC[C@H]1c1nc(C(=O)c2ccc(F)cc2)cs1)C1CCCCC1 ; CN[C@@H](C)C(=O)N[C@H](C(=O)N1CCC[C@H]1C(=O)Nc1sc(-c2ncco2)nc1-c1ccccc1)C1CCCCC1 ; CN[C@@H](C)C(=O)N[C@H](C(=O)N1CCC[C@H]1C(=O)Nc1snnc1-c1ccccc1)C1CCCCC1 ; CN[C@@H](C)C(=O)N[C@H]1CN(C(=O)CC(C)C)CC[C@H]2CC[C@@H](C(=O)NC(c3ccccc3)c3ccccc3)N2C1=O]

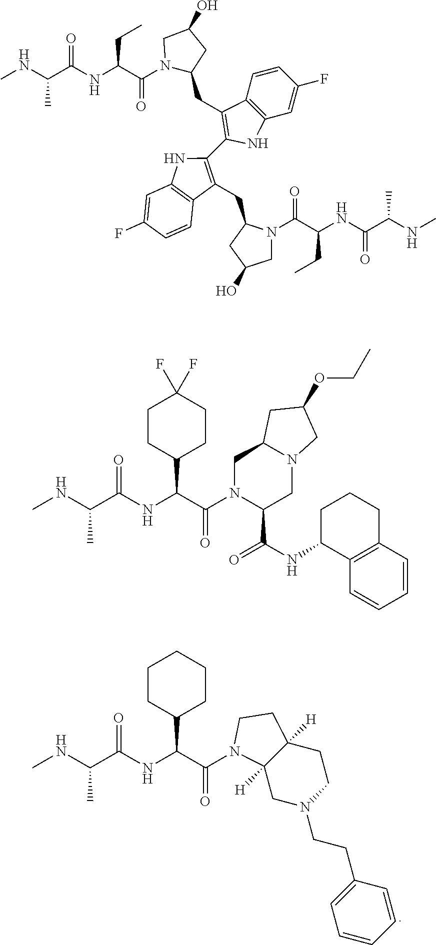

The term “independently” is used herein to indicate that the variable, which is independently applied, varies independently from application to application.

The term “alkyl” shall mean within its context a linear, branch-chained or cyclic fully saturated hydrocarbon radical or alkyl group, preferably a C₁-C₁₀, more preferably a C₁-C₆, alternatively a C₁-C₃ alkyl group, which may be optionally substituted. Examples of alkyl groups are methyl, ethyl, n-butyl, sec-butyl, n-hexyl, n-heptyl, n-octyl, n-nonyl, n-decyl, isopropyl, 2-methylpropyl, cyclopropyl, cyclopropylmethyl, cyclobutyl, cyclopentyl, cyclopentylethyl, cyclohexylethyl and cyclohexyl, among others. In certain embodiments, the alkyl group is end-capped with a halogen group (At, Br, Cl, F, or I). In certain preferred embodiments, compounds according to the present disclosure which may be used to covalently bind to dehalogenase enzymes. These compounds generally contain a side chain (often linked through a polyethylene glycol group) which terminates in an alkyl group which has a halogen substituent (often chlorine or bromine) on its distal end which results in covalent binding of the compound containing such a moiety to the protein.

The term “Alkenyl” refers to linear, branch-chained or cyclic C₂-C₁₀ (preferably C₂-C₆) hydrocarbon radicals containing at least one C═C bond.

The term “Alkynyl” refers to linear, branch-chained or cyclic C₂-C₁₀ (preferably C₂-C₆) hydrocarbon radicals containing at least one CC bond.

The term “alkylene” when used, refers to a —(CH₂)ₙ— group (n is an integer generally from 0-6), which may be optionally substituted. When substituted, the alkylene group preferably is substituted on one or more of the methylene groups with a C₁-C₆ alkyl group (including a cyclopropyl group or a t-butyl group), but may also be substituted with one or more halo groups, preferably from 1 to 3 halo groups or one or two hydroxyl groups, O—(C₁-C₆ alkyl) groups or amino acid sidechains as otherwise disclosed herein. In certain embodiments, an alkylene group may be substituted with a urethane or alkoxy group (or other group) which is further substituted with a polyethylene glycol chain (of from 1 to 10, preferably 1 to 6, often 1 to 4 ethylene glycol units) to which is substituted (preferably, but not exclusively on the distal end of the polyethylene glycol chain) an alkyl chain substituted with a single halogen group, preferably a chlorine group. In still other embodiments, the alkylene (often, a methylene) group, may be substituted with an amino acid sidechain group such as a sidechain group of a natural or unnatural amino acid, for example, alanine, β-alanine, arginine, asparagine, aspartic acid, cysteine, cystine, glutamic acid, glutamine, glycine, phenylalanine, histidine, isoleucine, lysine, leucine, methionine, proline, serine, threonine, valine, tryptophan or tyrosine.

The term “unsubstituted” shall mean substituted only with hydrogen atoms. A range of carbon atoms which includes C₀ means that carbon is absent and is replaced with H. Thus, a range of carbon atoms which is C₀-C₆ includes carbons atoms of 1, 2, 3, 4, 5 and 6 and for C₀, H stands in place of carbon.

The term “substituted” or “optionally substituted” shall mean independently (i.e., where more than substituent occurs, each substituent is independent of another substituent) one or more substituents (independently up to five substituents, preferably up to three substituents, often 1 or 2 substituents on a moiety in a compound according to the present disclosure and may include substituents which themselves may be further substituted) at a carbon (or nitrogen) position anywhere on a molecule within context, and includes as substituents hydroxyl, thiol, carboxyl, cyano (C≡N), nitro (NO₂), halogen (preferably, 1, 2 or 3 halogens, especially on an alkyl, especially a methyl group such as a trifluoromethyl), an alkyl group (preferably, C₁-C₁₀, more preferably, C₁-C₆), aryl (especially phenyl and substituted phenyl for example benzyl or benzoyl), alkoxy group (preferably, C₁-C₆ alkyl or aryl, including phenyl and substituted phenyl), thioether (C₁-C₆ alkyl or aryl), acyl (preferably, C₁-C₆ acyl), ester or thioester (preferably, C₁-C₆ alkyl or aryl) including alkylene ester (such that attachment is on the alkylene group, rather than at the ester function which is preferably substituted with a C₁-C₆ alkyl or aryl group), preferably, C₁-C₆ alkyl or aryl, halogen (preferably, F or Cl), amine (including a five- or six-membered cyclic alkylene amine, further including a C₁-C₆ alkyl amine or a C₁-C₆ dialkyl amine which alkyl groups may be substituted with one or two hydroxyl groups) or an optionally substituted —N(C₀-C₆ alkyl)C(O)(O—C₁-C₆ alkyl) group (which may be optionally substituted with a polyethylene glycol chain to which is further bound an alkyl group containing a single halogen, preferably chlorine substituent), hydrazine, amido, which is preferably substituted with one or two C₁-C₆ alkyl groups (including a carboxamide which is optionally substituted with one or two C₁-C₆ alkyl groups), alkanol (preferably, C₁-C₆ alkyl or aryl), or alkanoic acid (preferably, C₁-C₆ alkyl or aryl). Substituents according to the present disclosure may include, for example —SiR₁R₂R₃ groups where each of R₁ and R₂ is as otherwise described herein and R₃ is H or a C₁-C₆ alkyl group, preferably R₁, R₂, R₃ in this context is a C₁-C₃ alkyl group (including an isopropyl or t-butyl group). Each of the above-described groups may be linked directly to the substituted moiety or alternatively, the substituent may be linked to the substituted moiety (preferably in the case of an aryl or heteraryl moiety) through an optionally substituted —(CH₂)ₘ— or alternatively an optionally substituted —(OCH₂)ₘ—, —(OCH₂CH₂)ₘ— or —(CH₂CH₂O)ₘ— group, which may be substituted with any one or more of the above-described substituents. Alkylene groups —(CH₂)ₘ— or —(CH₂)ₙ— groups or other chains such as ethylene glycol chains, as identified above, may be substituted anywhere on the chain. Preferred substituents on alkylene groups include halogen or C₁-C₆ (preferably C₁-C₃) alkyl groups, which may be optionally substituted with one or two hydroxyl groups, one or two ether groups (O—C₁-C₆ groups), up to three halo groups (preferably F), or a sidechain of an amino acid as otherwise described herein and optionally substituted amide (preferably carboxamide substituted as described above) or urethane groups (often with one or two C₀-C₆ alkyl substituents, which group(s) may be further substituted). In certain embodiments, the alkylene group (often a single methylene group) is substituted with one or two optionally substituted C₁-C₆ alkyl groups, preferably C₁-C₄ alkyl group, most often methyl or O-methyl groups or a sidechain of an amino acid as otherwise described herein. In the present disclosure, a moiety in a molecule may be optionally substituted with up to five substituents, preferably up to three substituents. Most often, in the present disclosure moieties which are substituted are substituted with one or two substituents.

The term “substituted” (each substituent being independent of any other substituent) shall also mean within its context of use C₁-C₆ alkyl, C₁-C₆ alkoxy, halogen, amido, carboxamido, sulfone, including sulfonamide, keto, carboxy, C₁-C₆ ester (oxyester or carbonylester), C₁-C₆ keto, urethane —O—C(O)—NR₁R₂ or —N(R₁)—C(O)—O—R₁, nitro, cyano and amine (especially including a C₁-C₆ alkylene-NR₁R₂, a mono- or di-C₁-C₆ alkyl substituted amines which may be optionally substituted with one or two hydroxyl groups). Each of these groups contain unless otherwise indicated, within context, between 1 and 6 carbon atoms. In certain embodiments, preferred substituents will include for example, —NH—, —NHC(O)—, —O—, ═O, —(CH₂)ₘ— (here, m and n are in context, 1, 2, 3, 4, 5 or 6), —S—, —S(O)—, SO₂— or —NH—C(O)—NH—, —(CH₂)ₙOH, —(CH₂)ₙSH, —(CH₂)ₙCOOH, C₁-C₆ alkyl, —(CH₂)ₙO—(C₁-C₆ alkyl), —(CH₂)ₙC(O)—(C₁-C₆ alkyl), —(CH₂)ₙOC(O)—(C₁-C₆ alkyl), —(CH₂)ₙC(O)O—(C₁-C₆ alkyl), —(CH₂)—NHC(O)—R₁, —(CH₂)ₙC(O)—NR₁R₂, —(OCH₂)ₙOH, —(CH₂O)ₙCOOH, C₁-C₆ alkyl, —(OCH₂)ₙO—(C₁-C₆ alkyl), —(CH₂O)ₙC(O)—(C₁-C₆ alkyl), —(OCH₂)ₙNHC(O)—R₁, —(CH₂O)ₙC(O)—NR₁R₂, —S(O)₂—R_{S}, —S(O)—R_{S} (R_{S} is C₁-C₆ alkyl or a —(CH₂)ₘ—NR₁R₂ group), NO₂, CN or halogen (F, Cl, Br, I, preferably F or Cl), depending on the context of the use of the substituent. R₁ and R₂ are each, within context, H or a C₁-C₆ alkyl group (which may be optionally substituted with one or two hydroxyl groups or up to three halogen groups, preferably fluorine). The term “substituted” shall also mean, within the chemical context of the compound defined and substituent used, an optionally substituted aryl or heteroaryl group or an optionally substituted heterocyclic group as otherwise described herein. Alkylene groups may also be substituted as otherwise disclosed herein, preferably with optionally substituted C₁-C₆ alkyl groups (methyl, ethyl or hydroxymethyl or hydroxyethyl is preferred, thus providing a chiral center), a sidechain of an amino acid group as otherwise described herein, an amido group as described hereinabove, or a urethane group O—C(O)—NR₁R₂ group where R₁ and R₂ are as otherwise described herein, although numerous other groups may also be used as substituents. Various optionally substituted moieties may be substituted with 3 or more substituents, preferably no more than 3 substituents and preferably with 1 or 2 substituents. It is noted that in instances where, in a compound at a particular position of the molecule substitution is required (principally, because of valency), but no substitution is indicated, then that substituent is construed or understood to be H, unless the context of the substitution suggests otherwise.

The term “aryl” or “aromatic”, in context, refers to a substituted (as otherwise described herein) or unsubstituted monovalent aromatic radical having a single ring (e.g., benzene, phenyl, benzyl) or condensed rings (e.g., naphthyl, anthracenyl, phenanthrenyl, etc.) and can be bound to the compound according to the present disclosure at any available stable position on the ring(s) or as otherwise indicated in the chemical structure presented. Other examples of aryl groups, in context, may include heterocyclic aromatic ring systems, “heteroaryl” groups having one or more nitrogen, oxygen, or sulfur atoms in the ring (monocyclic) such as imidazole, furyl, pyrrole, furanyl, thiene, thiazole, pyridine, pyrimidine, pyrazine, triazole, oxazole or fused ring systems such as indole, quinoline, indolizine, azaindolizine, benzofurazan, etc., among others, which may be optionally substituted as described above. Among the heteroaryl groups which may be mentioned include nitrogen-containing heteroaryl groups such as pyrrole, pyridine, pyridone, pyridazine, pyrimidine, pyrazine, pyrazole, imidazole, triazole, triazine, tetrazole, indole, isoindole, indolizine, azaindolizine, purine, indazole, quinoline, dihydroquinoline, tetrahydroquinoline, isoquinoline, dihydroisoquinoline, tetrahydroisoquinoline, quinolizine, phthalazine, naphthyridine, quinoxaline, quinazoline, cinnoline, pteridine, imidazopyridine, imidazotriazine, pyrazinopyridazine, acridine, phenanthridine, carbazole, carbazoline, pyrimidine, phenanthroline, phenacene, oxadiazole, benzimidazole, pyrrolopyridine, pyrrolopyrimidine and pyridopyrimidine; sulfur-containing aromatic heterocycles such as thiophene and benzothiophene; oxygen-containing aromatic heterocycles such as furan, pyran, cyclopentapyran, benzofuran and isobenzofuran; and aromatic heterocycles comprising 2 or more hetero atoms selected from among nitrogen, sulfur and oxygen, such as thiazole, thiadizole, isothiazole, benzoxazole, benzothiazole, benzothiadiazole, phenothiazine, isoxazole, furazan, phenoxazine, pyrazoloxazole, imidazothiazole, thienofuran, furopyrrole, pyridoxazine, furopyridine, furopyrimidine, thienopyrimidine and oxazole, among others, all of which may be optionally substituted.

The term “substituted aryl” refers to an aromatic carbocyclic group comprised of at least one aromatic ring or of multiple condensed rings at least one of which being aromatic, wherein the ring(s) are substituted with one or more substituents. For example, an aryl group can comprise a substituent(s) selected from: —(CH₂)ₙOH, —(CH₂)ₙ—O—(C₁-C₆)alkyl, —(CH₂)ₙ—O—(CH₂)ₙ—(C₁-C₆)alkyl, —(CH₂)ₙ—C(O)(C₀-C₆) alkyl, —(CH₂)ₙ—C(O)O(C₀-C₆)alkyl, —(CH₂)ₙ—OC(O)(C₀-C₆)alkyl, amine, mono- or di-(C₁-C₆ alkyl) amine wherein the alkyl group on the amine is optionally substituted with 1 or 2 hydroxyl groups or up to three halo (preferably F, Cl) groups, OH, COOH, C₁-C₆ alkyl, preferably CH₃, CF₃, OMe, OCF₃, NO₂, or CN group (each of which may be substituted in ortho-, meta- and/or para-positions of the phenyl ring, preferably para-), an optionally substituted phenyl group (the phenyl group itself is preferably connected to a PTM group, including a ULM group via a linker group), and/or at least one of F, Cl, OH, COOH, CH₃, CF₃, OMe, OCF₃, NO₂, or CN group (in ortho-, meta- and/or para-positions of the phenyl ring, preferably para-), a naphthyl group, which may be optionally substituted, an optionally substituted heteroaryl, preferably an optionally substituted isoxazole including a methylsubstituted isoxazole, an optionally substituted oxazole including a methylsubstituted oxazole, an optionally substituted thiazole including a methyl substituted thiazole, an optionally substituted isothiazole including a methyl substituted isothiazole, an optionally substituted pyrrole including a methylsubstituted pyrrole, an optionally substituted imidazole including a methylimidazole, an optionally substituted benzimidazole or methoxybenzylimidazole, an optionally substituted oximidazole or methyloximidazole, an optionally substituted diazole group, including a methyldiazole group, an optionally substituted triazole group, including a methylsubstituted triazole group, an optionally substituted pyridine group, including a halo-(preferably, F) or methylsubstitutedpyridine group or an oxapyridine group (where the pyridine group is linked to the phenyl group by an oxygen), an optionally substituted furan, an optionally substituted benzofuran, an optionally substituted dihydrobenzofuran, an optionally substituted indole, indolizine or azaindolizine (2, 3, or 4-azaindolizine), an optionally substituted quinoline, and combinations thereof.

“Carboxyl” denotes the group —C(O)OR, where R is hydrogen, alkyl, substituted alkyl, aryl, substituted aryl, heteroaryl or substituted heteroaryl, whereas these generic substituents have meanings which are identical with definitions of the corresponding groups defined herein.

The term “heteroaryl” or “hetaryl” can mean but is in no way limited to an optionally substituted quinoline (which may be attached to the pharmacophore or substituted on any carbon atom within the quinoline ring), an optionally substituted indole (including dihydroindole), an optionally substituted indolizine, an optionally substituted azaindolizine (2, 3 or 4-azaindolizine) an optionally substituted benzimidazole, benzodiazole, benzoxofuran, an optionally substituted imidazole, an optionally substituted isoxazole, an optionally substituted oxazole (preferably methyl substituted), an optionally substituted diazole, an optionally substituted triazole, a tetrazole, an optionally substituted benzofuran, an optionally substituted thiophene, an optionally substituted thiazole (preferably methyl and/or thiol substituted), an optionally substituted isothiazole, an optionally substituted triazole (preferably a 1,2,3-triazole substituted with a methyl group, a triisopropylsilyl group, an optionally substituted —(CH₂)ₘ—O—C₁-C₆ alkyl group or an optionally substituted —(CH₂)ₘ—C(O)—O—C₁-C₆ alkyl group), an optionally substituted pyridine (2-, 3, or 4-pyridine) or a group according to the chemical structure:

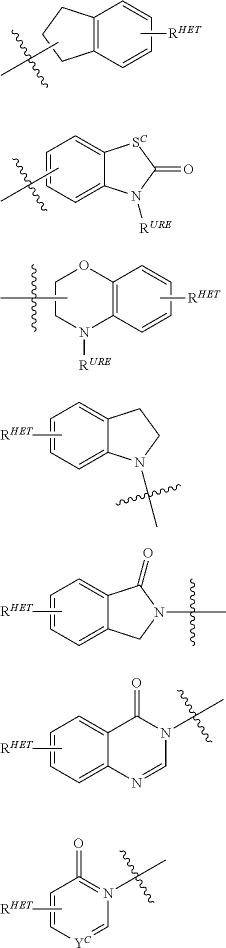

wherein:
S^{c} is CHR^{SS}, NR^{URE}, or O;
R^{HET} is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} is H, a C₁-C₆ alkyl (preferably H or C₁-C₃ alkyl) or a —C(O)(C₁-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted, and
Y^{c} is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl).

The terms “aralkyl” and “heteroarylalkyl” refer to groups that comprise both aryl or, respectively, heteroaryl as well as alkyl and/or heteroalkyl and/or carbocyclic and/or heterocycloalkyl ring systems according to the above definitions.

The term “arylalkyl” as used herein refers to an aryl group as defined above appended to an alkyl group defined above. The arylalkyl group is attached to the parent moiety through an alkyl group wherein the alkyl group is one to six carbon atoms. The aryl group in the arylalkyl group may be substituted as defined above.

The term “Heterocycle” refers to a cyclic group which contains at least one heteroatom, e.g., N, O or S, and may be aromatic (heteroaryl) or non-aromatic. Thus, the heteroaryl moieties are subsumed under the definition of heterocycle, depending on the context of its use. Exemplary heteroaryl groups are described hereinabove.

Exemplary heterocyclics include: azetidinyl, benzimidazolyl, 1,4-benzodioxanyl, 1,3-benzodioxolyl, benzoxazolyl, benzothiazolyl, benzothienyl, dihydroimidazolyl, dihydropyranyl, dihydrofuranyl, dioxanyl, dioxolanyl, ethyleneurea, 1,3-dioxolane, 1,3-dioxane, 1,4-dioxane, furyl, homopiperidinyl, imidazolyl, imidazolinyl, imidazolidinyl, indolinyl, indolyl, isoquinolinyl, isothiazolidinyl, isothiazolyl, isoxazolidinyl, isoxazolyl, morpholinyl, naphthyridinyl, oxazolidinyl, oxazolyl, pyridone, 2-pyrrolidone, pyridine, piperazinyl, N-methylpiperazinyl, piperidinyl, phthalimide, succinimide, pyrazinyl, pyrazolinyl, pyridyl, pyrimidinyl, pyrrolidinyl, pyrrolinyl, pyrrolyl, quinolinyl, tetrahydrofuranyl, tetrahydropyranyl, tetrahydroquinoline, thiazolidinyl, thiazolyl, thienyl, tetrahydrothiophene, oxane, oxetanyl, oxathiolanyl, thiane among others.

Heterocyclic groups can be optionally substituted with a member selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxy, carboxyalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SOaryl, —SO— heteroaryl, —SO2-alkyl, —SO2-substituted alkyl, —SO2-aryl, oxo (═O), and —SO2-heteroaryl. Such heterocyclic groups can have a single ring or multiple condensed rings. Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. The term “heterocyclic” also includes bicyclic groups in which any of the heterocyclic rings is fused to a benzene ring or a cyclohexane ring or another heterocyclic ring (for example, indolyl, quinolyl, isoquinolyl, tetrahydroquinolyl, and the like).

The term “cycloalkyl” can mean but is in no way limited to univalent groups derived from monocyclic or polycyclic alkyl groups or cycloalkanes, as defined herein, e.g., saturated monocyclic hydrocarbon groups having from three to twenty carbon atoms in the ring, including, but not limited to, cyclopropyl, cyclobutyl, cyclopentyl, cyclohexyl, cycloheptyl and the like. The term “substituted cycloalkyl” can mean but is in no way limited to a monocyclic or polycyclic alkyl group and being substituted by one or more substituents, for example, amino, halogen, alkyl, substituted alkyl, carbyloxy, carbylmercapto, aryl, nitro, mercapto or sulfo, whereas these generic substituent groups have meanings which are identical with definitions of the corresponding groups as defined in this legend.

“Heterocycloalkyl” refers to a monocyclic or polycyclic alkyl group in which at least one ring carbon atom of its cyclic structure being replaced with a heteroatom selected from the group consisting of N, O, S or P. “Substituted heterocycloalkyl” refers to a monocyclic or polycyclic alkyl group in which at least one ring carbon atom of its cyclic structure being replaced with a heteroatom selected from the group consisting of N, O, S or P and the group is containing one or more substituents selected from the group consisting of halogen, alkyl, substituted alkyl, carbyloxy, carbylmercapto, aryl, nitro, mercapto or sulfo, whereas these generic substituent group have meanings which are identical with definitions of the corresponding groups as defined in this legend.

The term “hydrocarbyl” shall mean a compound which contains carbon and hydrogen and which may be fully saturated, partially unsaturated or aromatic and includes aryl groups, alkyl groups, alkenyl groups and alkynyl groups.

The term “independently” is used herein to indicate that the variable, which is independently applied, varies independently from application to application.

The term “lower alkyl” refers to methyl, ethyl or propyl

The term “lower alkoxy” refers to methoxy, ethoxy or propoxy.

In any of the embodiments described herein, the W, X, Y, Z, G, G′, R, R′, R″, Q1-Q4, A, and Rn can independently be covalently coupled to a linker and/or a linker to which is attached one or more PTM, ULM, ILM or ILM′ groups.

Exemplary MLMs

In certain additional embodiments, the MLM of the bifunctional compound comprises chemical moieties such as substituted imidazolines, substituted spiro-indolinones, substituted pyrrolidines, substituted piperidinones, substituted morpholinones, substituted pyrrolopyrimidines, substituted imidazolopyridines, substituted thiazoloimidazoline, substituted pyrrolopyrrolidinones, and substituted isoquinolinones.

In additional embodiments, the MLM comprises the core structures mentioned above with adjacent bis-aryl substitutions positioned as cis- or trans-configurations.

In still additional embodiments, the MLM comprises part of structural features as in RG7112, RG7388, SAR405838, AMG-232, AM-7209, DS-5272, MK-8242, and NVP-CGM-097, and analogs or derivatives thereof.

In certain preferred embodiments, MLM is a derivative of substituted imidazoline represented as Formula (A-1), or thiazoloimidazoline represented as Formula (A-2), or spiro indolinone represented as Formula (A-3), or pyrollidine represented as Formula (A-4), or piperidinone/morphlinone represented as Formula (A-5), or isoquinolinone represented as Formula (A-6), or pyrollopyrimidine/imidazolopyridine represented as Formula (A-7), or pyrrolopyrrolidinone/imidazolopyrrolidinone represented as Formula (A-8).

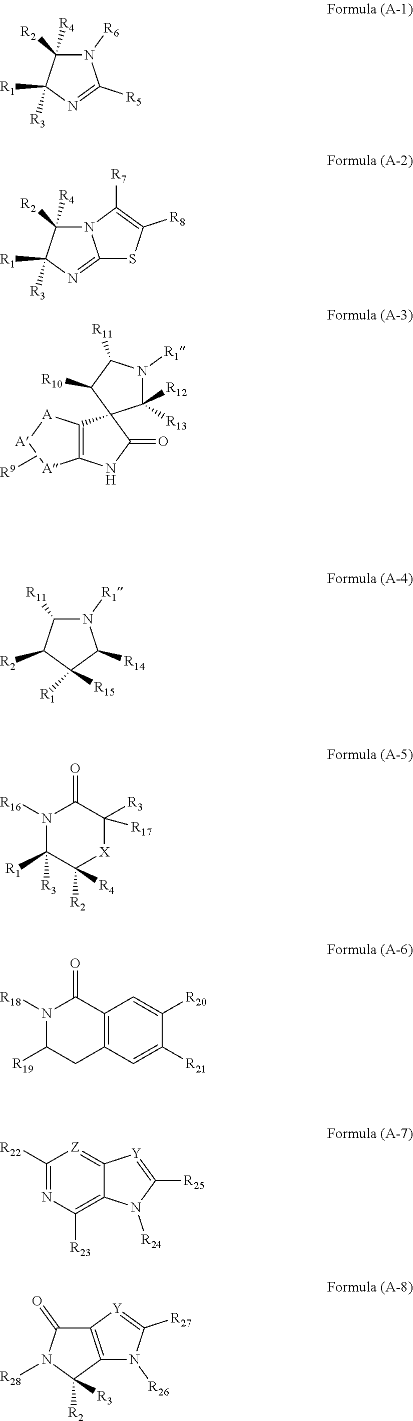

wherein above Formula (A-1) through Formula (A-8),
X of Formula (A-1) through Formula (A-8) is selected from the group consisting of carbon, oxygen, sulfur, sulfoxide, sulfone, and N—R^{a};
   R^{a} is independently H or an alkyl group with carbon number 1 to 6;
Y and Z of Formula (A-1) through Formula (A-8) are independently carbon or nitrogen;
A, A′ and A″ of Formula (A-1) through Formula (A-8) are independently selected from C, N, O or S, can also be one or two atoms forming a fused bicyclic ring, or a 6,5- and 5,5-fused aromatic bicyclic group;
R₁, R₂ of Formula (A-1) through Formula (A-8) are independently selected from the group consisting of an aryl or heteroaryl group, a heteroaryl group having one or two heteroatoms independently selected from sulfur or nitrogen, wherein the aryl or heteroaryl group can be mono-cyclic or bi-cyclic, or unsubstituted or substituted with one to three substituents independently selected from the group consisting of:
   halogen, —CN, C1 to C6 alkyl group, C3 to C6 cycloalkyl, —OH, alkoxy with 1 to 6 carbons, fluorine substituted alkoxy with 1 to 6 carbons, sulfoxide with 1 to 6 carbons, sulfone with 1 to 6 carbons, ketone with 2 to 6 carbons, amides with 2 to 6 carbons, and dialkyl amine with 2 to 6 carbons;
R₃, R₄ of Formula (A-1) through Formula (A-8) are independently selected from the group consisting of H, methyl and C1 to C6 alkyl;
R₅ of Formula (A-1) through Formula (A-8) is selected from the group consisting of an aryl or heteroaryl group, a heteroaryl group having one or two heteroatoms independently selected from sulfur or nitrogen, wherein the aryl or heteroaryl group can be mono-cyclic or bi-cyclic, or unsubstituted or substituted with one to three substituents independently selected from the group consisting of:
   halogen, —CN, C1 to C6 alkyl group, C3 to C6 cycloalkyl, —OH, alkoxy with 1 to 6 carbons, fluorine substituted alkoxy with 1 to 6 carbons, sulfoxide with 1 to 6 carbons, sulfone with 1 to 6 carbons, ketone with 2 to 6 carbons, amides with 2 to 6 carbons, dialkyl amine with 2 to 6 carbons, alkyl ether (C2 to C6), alkyl ketone (C3 to C6), morpholinyl, alkyl ester (C3 to C6), alkyl cyanide (C3 to C6);
R₆ of Formula (A-1) through Formula (A-8) is H or —C(═O)R^{b}, wherein
   R^{b} of Formula (A-1) through Formula (A-8) is selected from the group consisting of alkyl, cycloalkyl, mono-, di- or tri-substituted aryl or heteroaryl, 4-morpholinyl, 1-(3-oxopiperazunyl), 1-piperidinyl, 4-N—R^{e}-morpholinyl, 4-R^{e}-1-piperidinyl, and 3-R^{c}-1-piperidinyl, wherein
R^{c} of Formula (A-1) through Formula (A-8) is selected from the group consisting of alkyl, fluorine substituted alkyl, cyano alkyl, hydroxyl-substituted alkyl, cycloalkyl, alkoxyalkyl, amide alkyl, alkyl sulfone, alkyl sulfoxide, alkyl amide, aryl, heteroaryl, mono-, bis- and tri-substituted aryl or heteroaryl, CH2CH2R^{d}, and CH2CH2CH2R^{d}, wherein
R^{d} of Formula (A-1) through Formula (A-8) is selected from the group consisting of alkoxy, alkyl sulfone, alkyl sulfoxide, N-substituted carboxamide, —NHC(O)-alkyl, —NH—SO₂-alkyl, aryl, substituted aryl, heteroaryl, substituted heteroaryl;
R₇ of Formula (A-1) through Formula (A-8) is selected from the group consisting of H, C1 to C6 alkyl, cyclic alkyl, fluorine substituted alkyl, cyano substituted alkyl, 5- or 6-membered hetero aryl or aryl, substituted 5- or 6-membered hetero aryl or aryl;
R₈ of Formula (A-1) through Formula (A-8) is selected from the group consisting of —R^{e}—C(O)—R^{f}, —R^{e}-alkoxy, —R^{e}-aryl, —R^{e}-heteroaryl, and —R^{e}—C(O)—R^{f}—C(O)—R^{g}, wherein:
   R^{e} of Formula (A-1) through Formula (A-8) is an alkylene with 1 to 6 carbons, or a bond;
   R^{f} of Formula (A-1) through Formula (A-8) is a substituted 4- to 7-membered heterocycle;
R^{g} of Formula (A-1) through Formula (A-8) is selected from the group consisting of aryl, hetero aryl, substituted aryl or heteroaryl, and 4- to 7-membered heterocycle;
R₉ of Formula (A-1) through Formula (A-8) is selected from the group consisting of a mono-, bis- or tri-substituent on the fused bicyclic aromatic ring in Formula (A-3), wherein the substituents are independently selected from the group consisting of halogen, alkene, alkyne, alkyl, unsubstituted or substituted with Cl or F;
R₁₀ of Formula (A-1) through Formula (A-8) is selected from the group consisting of an aryl or heteroaryl group, wherein the heteroaryl group can contain one or two heteroatoms as sulfur or nitrogen, aryl or heteroaryl group can be mono-cyclic or bi-cyclic, the aryl or heteroaryl group can be unsubstituted or substituted with one to three substituents, including a halogen, F, Cl, —CN, alkene, alkyne, C1 to C6 alkyl group, C1 to C6 cycloalkyl, —OH, alkoxy with 1 to 6 carbons, fluorine substituted alkoxy with 1 to 6 carbons, sulfoxide with 1 to 6 carbons, sulfone with 1 to 6 carbons, ketone with 2 to 6 carbons;
R₁₁ of Formula (A-1) through Formula (A-8) is —C(O)—N(R^{h})(Rⁱ), wherein R^{h} and Rⁱ are selected from groups consisting of the following:
   H, C1 to C6 alkyl, alkoxy substituted alkyl, sulfone substituted alkyl, aryl, heterol aryl, mono-, bis- or tri-substituted aryl or hetero aryl, alkyl carboxylic acid, heteroaryl carboxylic acid, alkyl carboxylic acid, fluorine substituted alkyl carboxylic acid, aryl substituted cycloalkyl, hetero aryl substituted cycloalkyl; wherein
   R^{h} and Rⁱ of Formula (A-1) through Formula (A-8) are independently selected from the group consisting of H, connected to form a ring, 4-hydroxycyclohehexane; mono- and di-hydroxy substituted alkyl (C3 to C6); 3-hydroxycyclobutane; phenyl-4-carboxylic acid, and substituted phenyl-4-carboxylic acid;
R₁₂ and R₁₃ of Formula (A-1) through Formula (A-8) are independently selected from H, lower alkyl (C1 to C6), lower alkenyl (C2 to C6), lower alkynyl (C2 to C6), cycloalkyl (4, 5 and 6-membered ring), substituted cycloalkyl, cycloalkenyl, substituted cycloalkenyl, 5- and 6-membered aryl and heteroaryl, R12 and R13 can be connected to form a 5- and 6-membered ring with or without substitution on the ring;
R₁₄ of Formula (A-1) through Formula (A-8) is selected from the group consisting of alkyl, substituted alkyl, alkenyl, substituted alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R₁₅ of Formula (A-1) through Formula (A-8) is CN;
R₁₆ of Formula (A-1) through Formula (A-8) is selected from the group consisting of C1-6 alkyl, C1-6 cycloalkyl, C2-6 alkenyl, C1-6 alkyl or C3-6 cycloalkyl with one or multiple hydrogens replaced by fluorine, alkyl or cycloalkyl with one CH₂ replaced by S(═O), —S, or —S(═O)₂, alkyl or cycloalkyl with terminal CH₃ replaced by S(═O)₂N(alkyl)(alkyl), —C(═O)N(alkyl)(alkyl), —N(alkyl)S(═O)₂(alkyl), —C(═O)2(alkyl), —O(alkyl), C1-6 alkyl or alkyl-cycloalkyl with hydron replaced by hydroxyl group, a 3 to 7 membered cycloalkyl or heterocycloalkyl, optionally containing a —(C=0)-group, or a 5 to 6 membered aryl or heteroaryl group, which heterocycloalkyl or heteroaryl group can contain from one to three heteroatoms independently selected from O, N or S, and the cycloalkyl, heterocycloalkyl, aryl or heteroaryl group can be unsubstituted or substituted with from one to three substituents independently selected from halogen, C1-6 alkyl groups, hydroxylated C1-6 alkyl, C1-6 alkyl containing thioether, ether, sulfone, sulfoxide, fluorine substituted ether or cyano group;

R₁₇ of Formula (A-1) through Formula (A-8) is selected from the group consisting of (CH₂)nC(O)NR^{k}R^{l}, wherein R^{k} and R^{l} are independently selected from H, C1-6 alkyl, hydroxylated C1-6 alkyl, C1-6 alkoxy alkyl, C1-6 alkyl with one or multiple hydrogens replaced by fluorine, C1-6 alkyl with one carbon replaced by S(O), S(O)(O), C1-6 alkoxyalkyl with one or multiple hydrogens replaced by fluorine, C1-6 alkyl with hydrogen replaced by a cyano group, 5 and 6 membered aryl or heteroaryl, aklyl aryl with alkyl group containing 1-6 carbons, and alkyl heteroaryl with alkyl group containing 1-6 carbons, wherein the aryl or heteroaryl group can be further substituted;
R₁₈ of Formula (A-1) through Formula (A-8) is selected from the group consisting of substituted aryl, heteroaryl, alkyl, cycloalkyl, the substitution is preferably —N(C1-4 alkyl)(cycloalkyl), —N(C1-4 alkyl)alkyl-cycloalkyl, and —N(C1-4 alkyl)[(alkyl)-(heterocycle-substituted)-cycloalkyl];
R₁₉ of Formula (A-1) through Formula (A-8) is selected from the group consisting of aryl, heteroaryl, bicyclic heteroaryl, and these aryl or hetroaryl groups can be substituted with halogen, C1-6 alkyl, C1-6 cycloalkyl, CF₃, F, CN, alkyne, alkyl sulfone, the halogen substitution can be mono- bis- or tri-substituted;
R₂₀ and R₂₁ of Formula (A-1) through Formula (A-8) are independently selected from C1-6 alkyl, C1-6 cycloalkyl, C1-6 alkoxy, hydroxylated C1-6 alkoxy, and fluorine substituted C1-6 alkoxy, wherein R₂₀ and R₂₁ can further be connected to form a 5, 6 and 7-membered cyclic or heterocyclic ring, which can further be substituted;
R₂₂ of Formula (A-1) through Formula (A-8) is selected from the group consisting of H, C1-6 alkyl, C1-6 cycloalkyl, carboxylic acid, carboxylic acid ester, amide, reverse amide, sulfonamide, reverse sulfonamide, N-acyl urea, nitrogen-containing 5-membered heterocycle, the 5-membered heterocycles can be further substituted with C1-6 alkyl, alkoxy, fluorine-substituted alkyl, CN, and alkylsulfone;
R₂₃ of Formula (A-1) through Formula (A-8) is selected from aryl, heteroaryl, —O-aryl, —O— heteroaryl, —O-alkyl, —O-alkyl-cycloalkyl, —NH-alkyl, —NH-alkyl-cycloalkyl, —N(H)-aryl, —N(H)-heteroaryl, —N(alkyl)-aryl, —N(alkyl)-heteroaryl, the aryl or heteroaryl groups can be substituted with halogen, C1-6 alkyl, hydroxylated C1-6 alkyl, cycloalkyl, fluorine-substituted C1-6 alkyl, CN, alkoxy, alkyl sulfone, amide and sulfonamide;
R₂₄ of Formula (A-1) through Formula (A-8) is selected from the group consisting of —CH2-(C1-6 alkyl), —CH2-cycloalkyl, —CH2-aryl, CH2-heteroaryl, where alkyl, cycloalkyl, aryl and heteroaryl can be substituted with halogen, alkoxy, hydroxylated alkyl, cyano-substituted alkyl, cycloalyl and substituted cycloalkyl;
R₂₅ of Formula (A-1) through Formula (A-8) is selected from the group consisting of C1-6 alkyl, C1-6 alkyl-cycloalkyl, alkoxy-substituted alkyl, hydroxylated alkyl, aryl, heteroaryl, substituted aryl or heteroaryl, 5,6, and 7-membered nitrogen-containing saturated heterocycles, 5,6-fused and 6,6-fused nitrogen-containing saturated heterocycles and these saturated heterocycles can be substituted with C1-6 alkyl, fluorine-substituted C1-6 alkyl, alkoxy, aryl and heteroaryl group;
R₂₆ of Formula (A-1) through Formula (A-8) is selected from the group consisting of C1-6 alkyl, C3-6 cycloalkyl, the alkyl or cycloalkyl can be substituted with —OH, alkoxy, fluorine-substituted alkoxy, fluorine-substituted alkyl, —NH₂, —NH-alkyl, NH—C(O)alkyl, —NH—S(O)₂-alkyl, and —S(O)₂-alkyl;
R₂₇ of Formula (A-1) through Formula (A-8) is selected from the group consisting of aryl, heteroaryl, bicyclic heteroaryl, wherein the aryl or heteroaryl groups can be substituted with C1-6 alkyl, alkoxy, NH2, NH-alkyl, halogen, or —CN, and the substitution can be independently mono-, bis- and tri-substitution;
R₂₈ of Formula (A-1) through Formula (A-8) is selected from the group consisting of aryl, 5 and 6-membered heteroaryl, bicyclic heteroaryl, cycloalkyl, saturated heterocycle such as piperidine, piperidinone, tetrahydropyran, N-acyl-piperidine, wherein the cycloalkyl, saturated heterocycle, aryl or heteroaryl can be further substituted with —OH, alkoxy, mono-, bis- or tri-substitution including halogen, —CN, alkyl sulfone, and fluorine substituted alkyl groups; and
R_{1″} of Formula (A-1) through Formula (A-8) is selected from the group consisting of alkyl, aryl substituted alkyl, alkoxy substituted alkyl, cycloalkyl, aryl-substituted cycloalkyl, and alkoxy substituted cycloalkyl.

In certain embodiments, the heterocycles in R^{f} and R^{g} of Formula (A-1) through Formula (A-8) are substituted pyrrolidine, substituted piperidine, substituted piperizine.

More specifically, non-limiting examples of MLMs include those shown below as well as those ‘hybrid’ molecules that arise from the combination of 1 or more of the different features shown in the molecules below.

Using MLM in Formula A-1 through A-8, the following PROTACs can be prepared to target a particular protein for degradation, where ‘L′’ is a connector (i.e. a linker group), and “PTM” is a ligand binding to a target protein.

In certain embodiments, the description provides a bifunctional molecule comprising a structure selected from the group consisting of:

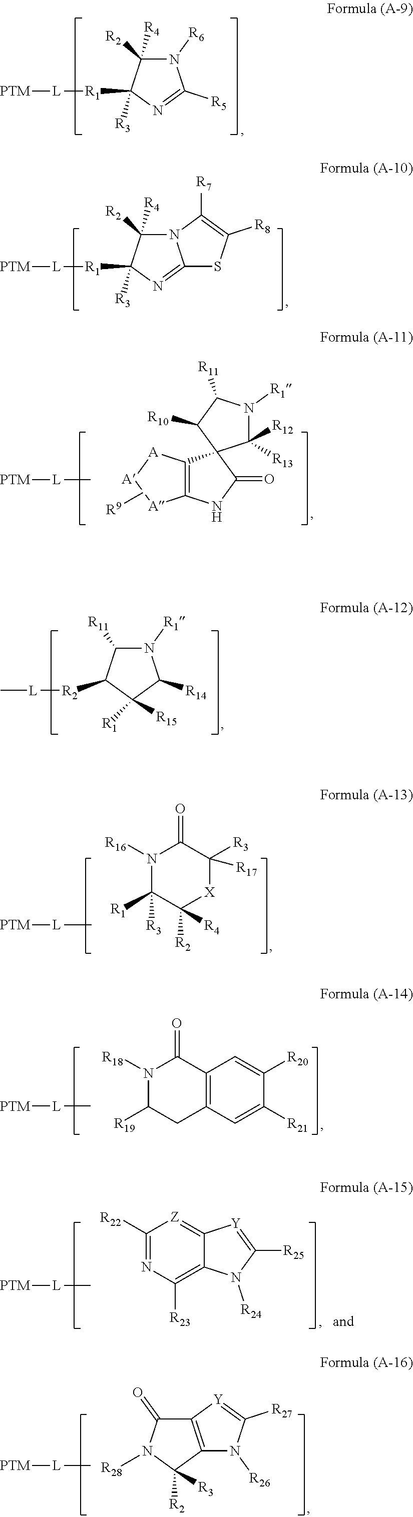

wherein X, R^{a}, Y, Z, A, A′, A″, R₁, R₂, R₃, R₄, R₅, R₆, R^{b}, R^{c}, R^{d}, R₇, R^{e}, R^{f}, R^{g}, R₉, R₁₀, R₁₁, R₁₂, R₁₃, R₁₄, R₁₅, R₁₆, R₁₇, R^{k}, R^{l}, R₁₈, R₁₉, R₂₀, R₂₁, R₂₂, R₂₃, R₂₄, R₂₅, R₂₆, R₂₇, R₂₈, and R_{1″} are as defined herein with regard to Formulas (A-1) through (A-8).

In certain embodiments, the description provides bifunctional or chimeric molecules with the structure: PTM-L-MLM, wherein PTM is a protein target binding moiety coupled to an MLM by L, wherein L is a bond (i.e., absent) or a chemical linker. In certain embodiments, the MLM has a structure selected from the group consisting of A-1-1, A-1-2, A-1-3, and A-1-4:

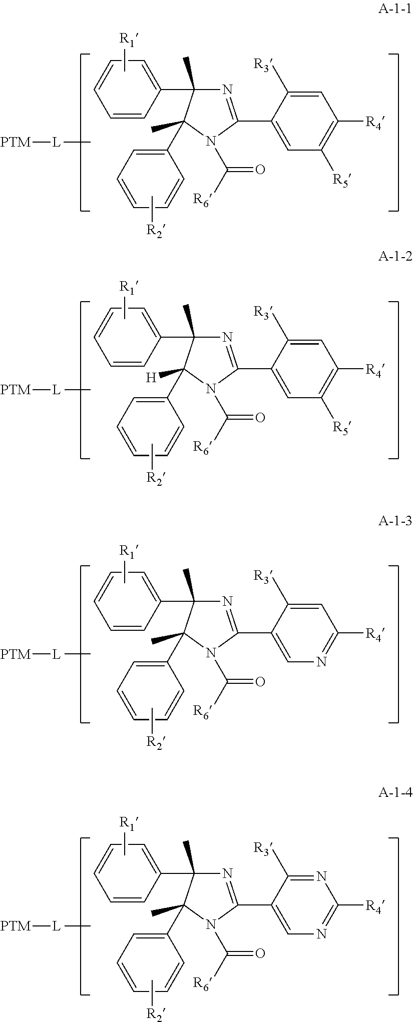

wherein:
R1′ and R2′ of Formulas A-1-1 through A-1-4 (i.e., A-1-1, A-1-2, A-1-3, and A-1-4) are independently selected from the group consisting of F, Cl, Br, I, acetylene, CN, CF₃ and NO₂;
R3′ is selected from the group consisting of —OCH₃, —OCH₂CH₃, —OCH₂CH₂F, —OCH₂CH₂OCH₃, and —OCH(CH₃)₂;
R4′ of Formulas A-1-1 through A-1-4 is selected from the group consisting of H, halogen, —CH₃, —CF₃, —OCH₃, —C(CH₃)₃, —CH(CH₃)₂, -cyclopropyl, —CN, —C(CH₃)₂OH, —C(CH₃)2OCH₂CH₃, —C(CH₃)₂CH₂OH, —C(CH₃)₂CH₂OCH₂CH₃, —C(CH₃)₂CH₂OCH₂CH₂OH, —C(CH₃)₂CH₂OCH₂CH₃, —C(CH₃)2CN, —C(CH₃)₂C(O)CH₃, —C(CH₃)₂C(O)NHCH₃, —C(CH₃)₂C(O)N(CH₃)₂, —SCH₃, —SCH₂CH₃, —S(O)₂CH₃, —S(O₂)CH₂CH₃, —NHC(CH₃)₃, —N(CH₃)₂, pyrrolidinyl, and 4-morpholinyl;
R5′ of Formulas A-1-1 through A-1-4 is selected from the group consisting of halogen, -cyclopropyl, —S(O)₂CH₃, —S(O)₂CH₂CH₃, 1-pyrrolidinyl, —NH₂, —N(CH₃)₂, and —NHC(CH₃)₃; and
R6′ of Formulas A-1-1 through A-1-4 is selected from the structures presented below where the linker connection point is indicated as “*”.
Beside R6′ as the point for linker attachment, R4′ can also serve as the linker attachment position. In the case that R4′ is the linker connection site, linker will be connected to the terminal atom of R4′ groups shown above.

In certain embodiments, the linker connection position of Formulas A-1-1 through A-1-4 is at least one of R4′ or R6′ or both.

In certain embodiments, R6′ of Formulas A-1-1 through A-1-4 is independently selected from the group consisting of H,

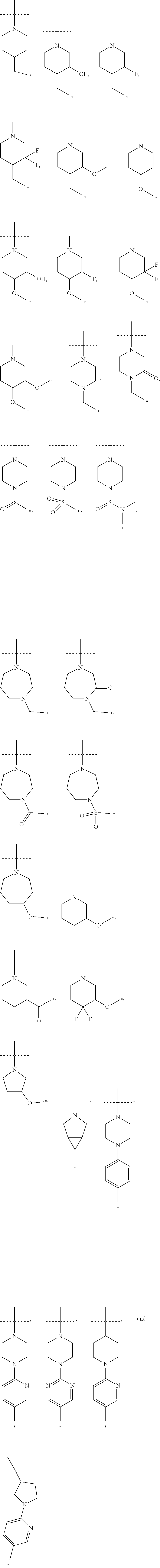

wherein “*” indicates the point of attachment of the linker.

In certain embodiments, the linker of Formula A-4-1 through A-4-6 is attached to at least one of R1′, R2′, R3′, R4′, R5′, R6′, or a combination thereof.

In certain embodiments, the description provides bifunctional or chimeric molecules with the structure: PTM-L-MLM, wherein PTM is a protein target binding moiety coupled to an MLM by L, wherein L is a bond (i.e., absent) or a chemical linker. In certain embodiments, the MLM has a structure selected from the group consisting of A-4-1, A-4-2, A-4-3, A-4-4, A-4-5, and A-4-6:

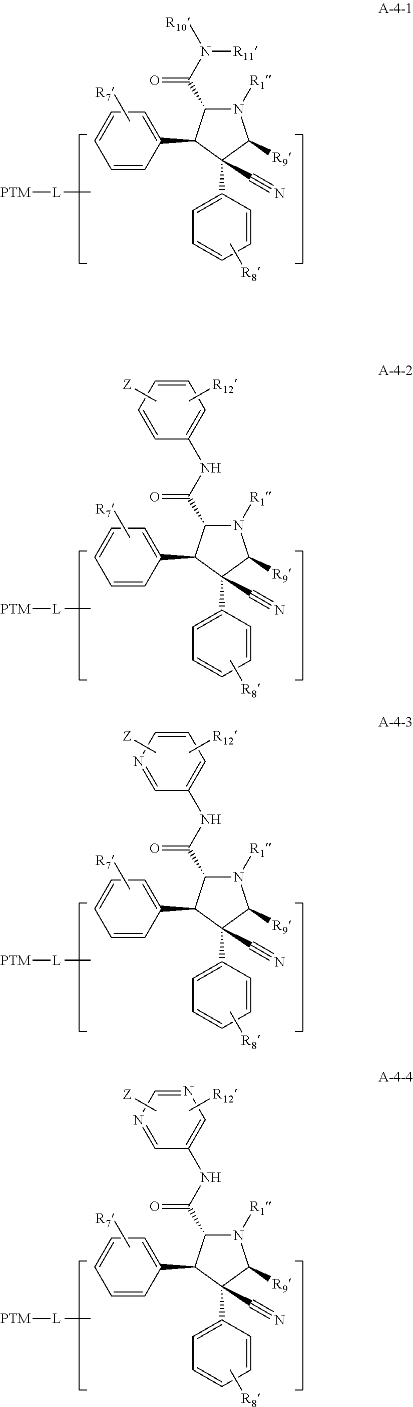

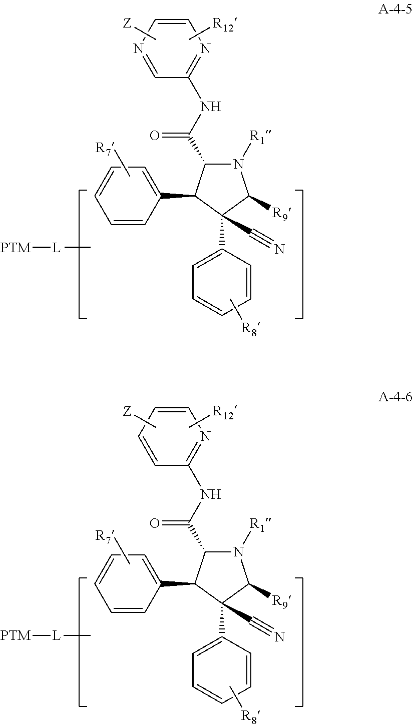

wherein:
R7′ of Formula A-4-1 through A-4-6 (i.e., A-4-1, A-4-2, A-4-3, A-4-4, A-4-5, and A-4-6) is a member selected from the group consisting of halogen, mono-, and di- or tri-substituted halogen;
R8′ of Formula A-4-1 through A-4-6 is selected from the group consisting of H, —F, —Cl, —Br, —I, —CN, —NO₂, ethylnyl, cyclopropyl, methyl, ethyl, isopropyl, vinyl, methoxy, ethoxy, isopropoxy, —OH, other C1-6 alkyl, other C1-6 alkenyl, and C1-6 alkynyl, mono-, di- or tri-substituted;
R9′ of Formula A-4-1 through A-4-6 is selected from the group consisting of alkyl, substituted alkyl, alkenyl, substituted alkenyl, alkynyl, substituted alkynyl, aryl, substituted aryl, hetero aryl, substituted heteroaryl, cycloalkyl, substituted cycloalkyl, alkenyl, and substituted cycloalkenyl;
Z of Formula A-4-1 through A-4-6 is selected from the group consisting of H, —OCH₃, —OCH₂CH₃, and halogen;
R10′ and R11′ of Formula A-4-1 through A-4-6 are each independently selected from the group consisting of H, (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH)ₙ—CONR′R″, (CH₂)ₙ—OR′, (CH₂)ₙ—SR′, (CH₂)ₙ—SOR′, (CH₂)ₙ—CH(OH)—R′, (CH₂)ₙ—COR′, (CH₂)ₙ—SO₂R′, (CH₂)ₙ—SONR′R″, (CH₂)ₙ—SO₂NR′R″, (CH₂CH₂O)ₘ—(CH₂)ₙ—R′, (CH₂CH2O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ(CH₂)ₙ—NR′SO₂R″, (CH₂CH₂O)ₘ(CH₂)ₙ—COOH, (CH₂CH₂O)ₘ(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)p-(CH₂CH₂O)ₘ (CH₂)ₙR′, (CH₂)ₚ—(CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ—(CH₂)ₙ—COOR′, (CH₂)ₚ—(CH₂CH₂O)ₘ—(CH₂)ₙ—CONR′R″, (CH₂)p-(CH₂CH₂O)ₘ—(CH₂)ₙ—SO₂R′, (CH2)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, Aryl-(CH₂)ₙ—COOH, and heteroaryl-alkyl-CO-alkyl-NR′R″m, wherein the alkyl may be substituted with OR′, and heteroaryl-(CH₂)ₙ-heterocycle wherein the heterocycle may optionally be substituted with alkyl, hydroxyl, COOR′ and COR′; wherein R′ and R″ are selected from H, alkyl, alkyl substituted with halogen, hydroxyl, NH2, NH(alkyl), N(alkyl)₂, oxo, carboxy, cycloalkyl and heteroaryl;
m, n, and p are independently 0 to 6;
R12′ of Formula A-4-1 through A-4-6 is selected from the group consisting of —O-(alkyl), —O-(alkyl)-akoxy, —C(O)-(alkyl), —C(OH)-alkyl-alkoxy, —C(O)—NH-(alkyl), —C(O)—N-(alkyl)₂, —S(O)-(alkyl), S(O)₂-(alkyl), —C(O)-(cyclic amine), and —O-aryl-(alkyl), —O-aryl-(alkoxy);
R1″ of Formula A-4-1 through A-4-6 is selected from the group consisting of alkyl, aryl substituted alkyl, aloxy substituted alkyl, cycloalkyl, ary-substituted cycloalkyl, and alkoxy substituted cycloalkyl.

In any of the aspects or embodiments described herein, the alkyl, alkoxy or the like can be a lower alkyl or lower alkoxy.

In certain embodiments, the linker connection position of Formula A-4-1 through A-4-6 is at least one of Z, R8′, R9′, R10′, R11″, R12″, or R1″.

The method used to design chimeric molecules as presented in A-1-1 through A-1-4, A-4-1 through A-4-6 can be applied to MLM with formula A-2, A-3, A-5, A-6, A-7 and A-8, wherein the solvent exposed area in the MLM can be connected to linker “L” which will be attached to target protein ligand “PTM”, to construct PROTACs.

Exemplary MDM2 binding moieties include, but not limited, the following:

1. The HDM2/MDM2 inhibitors identified in Vassilev, et al., In vivo activation of the p53 pathway by small-molecule antagonists of MDM2, SCIENCE vol: 303, page: 844-848 (2004), and Schneekloth, et al., Targeted intracellular protein degradation induced by a small molecule: En route to chemical proteomics, Bioorg. Med. Chem. Lett. 18 (2008) 5904-5908, including (or additionally) the compounds nutlin-3, nutlin-2, and nutlin-1 (derivatized) as described below, as well as all derivatives and analogs thereof:

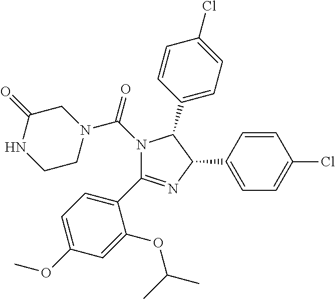 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]

(derivatized where a linker group L or a -(L-MLM) group is attached, for example, at the methoxy group or as a hydroxyl group);

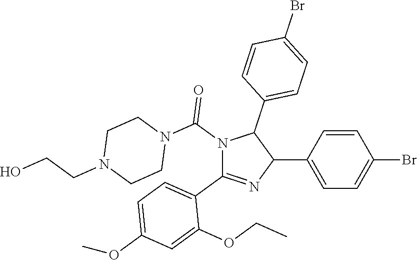 [SMILES: CCOc1cc(OC)ccc1C1=NC(c2ccc(Br)cc2)C(c2ccc(Br)cc2)N1C(=O)N1CCN(CCO)CC1]

(derivatized where a linker group L or a -(L-MLM) group is attached, for example, at the methoxy group or hydroxyl group);

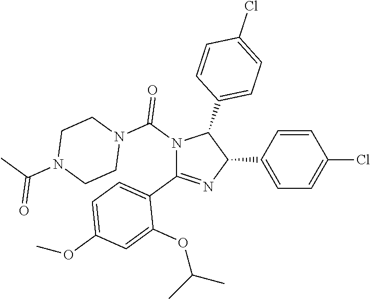 [SMILES: COc1ccc(C2=N[C@@H](c3ccc(Cl)cc3)[C@@H](c3ccc(Cl)cc3)N2C(=O)N2CCN(C(C)=O)CC2)c(OC(C)C)c1]

(derivatized where a linker group L or a -(L-MLM) group is attached, for example, via the methoxy group or as a hydroxyl group); and

2. Trans-4-Iodo-4′-Boranyl-Chalcone

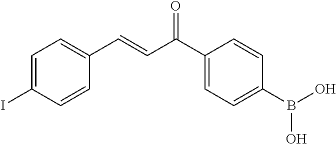 [SMILES: O=C(/C=C/c1ccc(I)cc1)c1ccc(B(O)O)cc1]

(derivatized where a linker group L or a a linker group L or a-(L-MLM) group is attached, for example, via a hydroxy group).

Exemplary CLMs

Neo-Imide Compounds

In one aspect the description provides compounds useful for binding and/or inhibiting cereblon. In certain embodiments, the compound is selected from the group consisting of chemical structures:

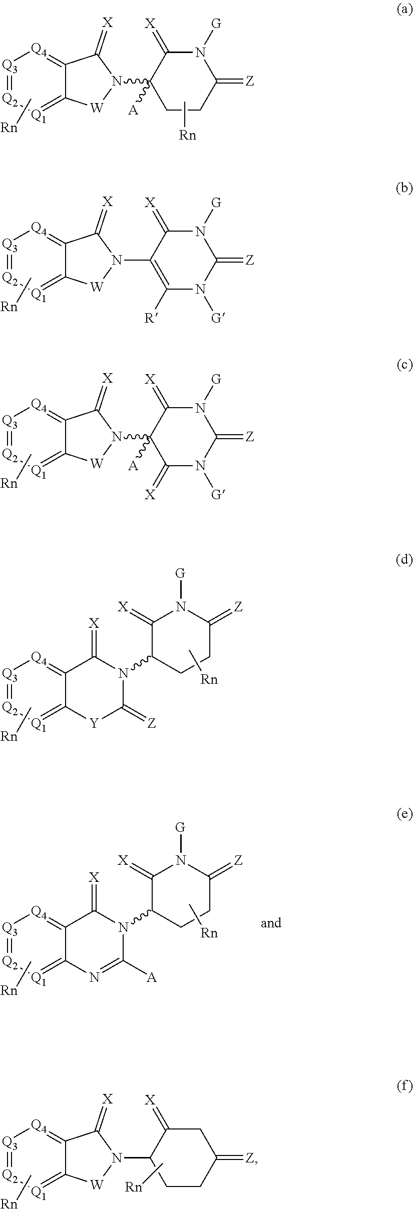

wherein:
W of Formulas (a) through (e) is independently selected from the group CH₂, CHR, C═O, SO₂, NH, and N-alkyl;
X of Formulas (a) through (e) is independently selected from the group O, S and H₂,
Y of Formulas (a) through (e) is independently selected from the group CH₂, —C═CR′, NH, N-alkyl, N-aryl, N-hetaryl, N-cycloalkyl, N-heterocyclyl, O, and S;
Z of Formulas (a) through (e) is independently selected from the group O, and S or H₂ except that both X and Z cannot be H₂,
G and G′ of Formulas (a) through (e) are independently selected from the group H, optionally substituted linear or branched alkyl, OH, R′OCOOR, R′OCONRR″, CH₂-heterocyclyl optionally substituted with R′, and benzyl optionally substituted with R′;
Q1-Q4 of Formulas (a) through (e) represent a carbon C substituted with a group independently selected from R′, N or N-oxide;
A of Formulas (a) through (e) is independently selected from the group optionally substituted linear or branched alkyl, cycloalkyl, Cl and F;
R of Formulas (a) through (e) comprises, but is not limited to: —CONR′R″, —OR′, —NR′R″, —SR′, —SO₂R′, —SO₂NR′R″, —CR′R″—, —CR′NR′R″—, (—CR′O)ₙ′R″, -aryl, -hetaryl, optionally substituted linear or branched alkyl, -cycloalkyl, -heterocyclyl, —P(O)(OR′)R″, —P(O)R′R″, —OP(O)(OR′)R″, —OP(O)R′R″, —Cl, —F, —Br, —I, —CF₃, —CN, —NR′SO₂NR′R″, —NR′CONR′R″, —CONR′COR″, —NR′C(═N—CN)NR′R″, —C(═N—CN)NR′R″, —NR′C(═N—CN)R″, —NR′C(═C—NO₂)NR′R″, —SO₂NR′COR″, —NO₂, —CO₂R′, —C(C═N—OR′)R″, —CR′═CR′R″, —CCR′, —S(C═O)(C═N—R′)R″, —SF₅ and —OCF₃
R′ and R″ of Formulas (a) through (e) are independently selected from a bond, H, alkyl, cycloalkyl, aryl, heteroaryl, heterocyclic, —C(═O)R, heterocyclyl, each of which is optionally substituted;
N′ of Formulas (a) through (e) is an integer from 1-10 (e.g. 1-4, 1, 2, 3, 4, 5, 6, 7, 8, 9, or 10);
[symbol] of Formulas (a) through (e) represents a bond that may be stereospecific ((R) or (S)) or non-stereospecific; and
Rₙ of Formulas (a) through (e) comprises from 1 to 4 independently selected functional groups or atoms, for example, O, OH, N, C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy.

Exemplary CLMs

In any of the compounds described herein, the CLM comprises a chemical structure selected from the group:

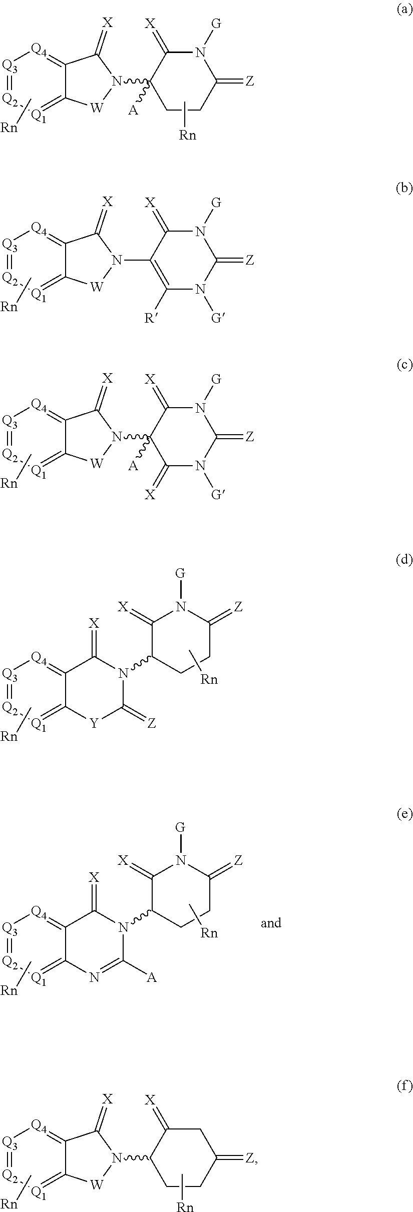

wherein:
W of Formulas (a) through (e) is independently selected from the group CH₂, CHR, C═O, SO₂, NH, and N-alkyl;
X of Formulas (a) through (e) is independently selected from the group O, S and H2;
Y of Formulas (a) through (e) is independently selected from the group CH₂, —C═CR′, NH, N-alkyl, N-aryl, N-hetaryl, N-cycloalkyl, N-heterocyclyl, O, and S;
Z of Formulas (a) through (e) is independently selected from the group O, and S or H2 except that both X and Z cannot be H2;
G and G′ of Formulas (a) through (e) are independently selected from the group H, optionally substituted linear or branched alkyl, OH, R′OCOOR, R′OCONRR″, CH₂-heterocyclyl optionally substituted with R′, and benzyl optionally substituted with R′;
Q1-Q4 of Formulas (a) through (e) represent a carbon C substituted with a group independently selected from R′, N or N-oxide;
A of Formulas (a) through (e) is independently selected from the group optionally substituted linear or branched alkyl, cycloalkyl, Cl and F;
R of Formulas (a) through (e) comprises, but is not limited to: —CONR′R″, —OR′, —NR′R″, —SR′, —SO2R′, —SO2NR′R″, —CR′R″—, —CR′NR′R″—, (—CR′O)ₙ′R″, -aryl, -hetaryl, optionally substituted linear or branched alkyl, -cycloalkyl, -heterocyclyl, —P(O)(OR′)R″, —P(O)R′R″, —OP(O)(OR′)R″, —OP(O)R′R″, —C1, —F, —Br, —I, —CF3, —CN, —NR′SO2NR′R″, —NR′CONR′R″, —CONR′COR″, —NR′C(═N—CN)NR′R″, —C(═N—CN)NR′R″, —NR′C(═N—CN)R″, —NR′C(═C—NO2)NR′R″, —SO2NR′COR″, —NO2, —CO2R′, —C(C═N—OR′)R″, —CR′═CR′R″, —CCR′, —S(C═O)(C═N—R′)R″, —SF5 and —OCF3
R′ and R″ of Formulas (a) through (e) are independently selected from a bond, H, alkyl, cycloalkyl, aryl, heteroaryl, heterocyclic, —C(═O)R, heterocyclyl, each of which is optionally substituted;
N′ of Formulas (a) through (e) is an integer from 1-10 (e.g., 1-4);
[symbol] of Formulas (a) through (e) represents a bond that may be stereospecific ((R) or (S)) or non-stereospecific; and
Rn of Formulas (a) through (e) comprises from 1 to 4 independently selected functional groups or atoms, for example, O, OH, N, C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy, and optionally, one of which is modified to be covalently joined to a PTM, a chemical linker group (L), a ULM, CLM (or CLM′) or combination thereof.

In certain embodiments described herein, the CLM or ULM comprises a chemical structure selected from the group:

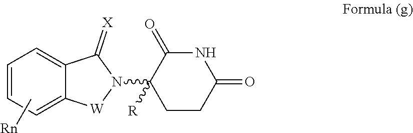

wherein:
W of Formula (g) is independently selected from the group CH₂, C═O, NH, and N-alkyl (e.g., C1-C6 alkyl (linear, branched, optionally substituted));
R of Formula (g) is independently selected from a H, methyl, or optionally substituted alkyl;
[symbol] of Formula (g) represents a bond that may be stereospecific ((R) or (S)) or non-stereospecific; and
Rn of Formula (g) comprises from 1 to 4 independently selected functional groups or atoms, for example, O, OH, N, C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy, and optionally, one of which is modified to be covalently joined to a PTM, a chemical linker group (L), a ULM, CLM (or CLM′) or combination thereof.

In any of the embodiments described herein, the W, X, Y, Z, G, G′, R, R′, R″, Q1-Q4, A, and Rn of Formulas (a) through (g) can independently be covalently coupled to a linker and/or a linker to which is attached one or more PTM, ULM, CLM or CLM′ groups.

In any of the aspects or embodiments described herein, Rn comprises from 1 to 4 independently selected functional groups or atoms, for example, O, OH, N, C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy, and optionally, one of which is modified to be covalently joined to a PTM, a chemical linker group (L), a ULM, CLM (or CLM′) or combination thereof.

More specifically, non-limiting examples of CLMs include those shown below as well as those “hybrid” molecules that arise from the combination of 1 or more of the different features shown in the molecules below.

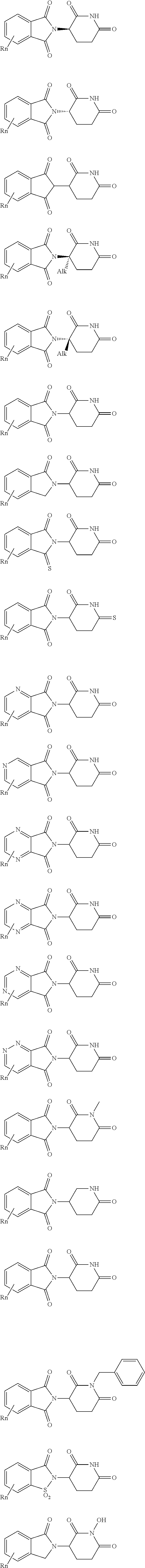

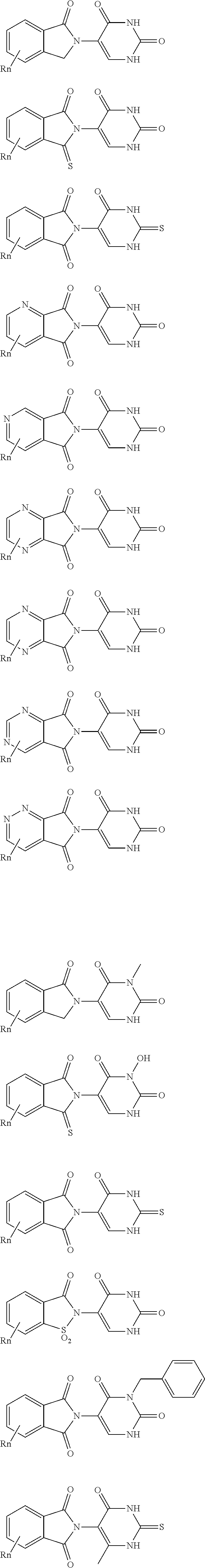

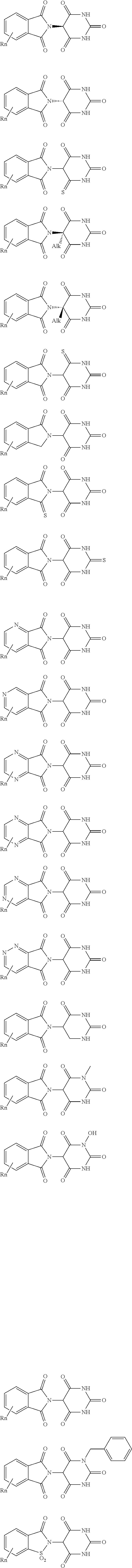

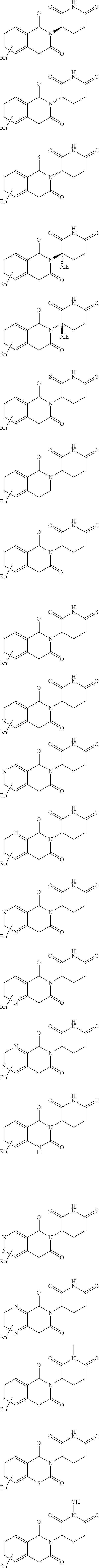

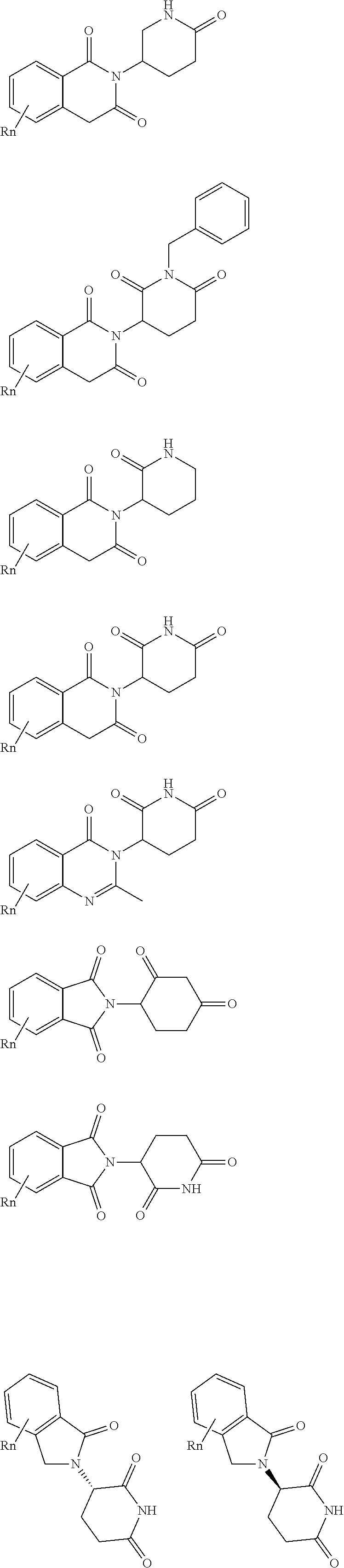

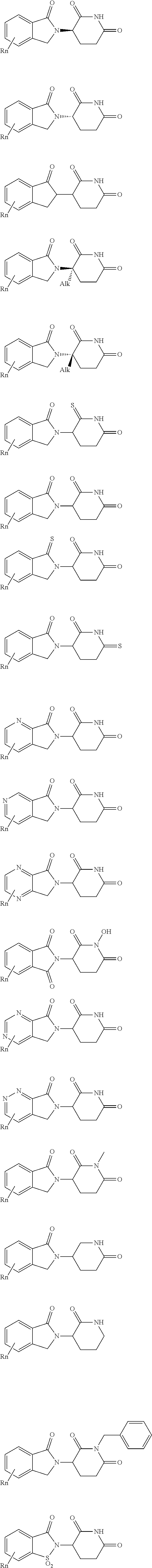

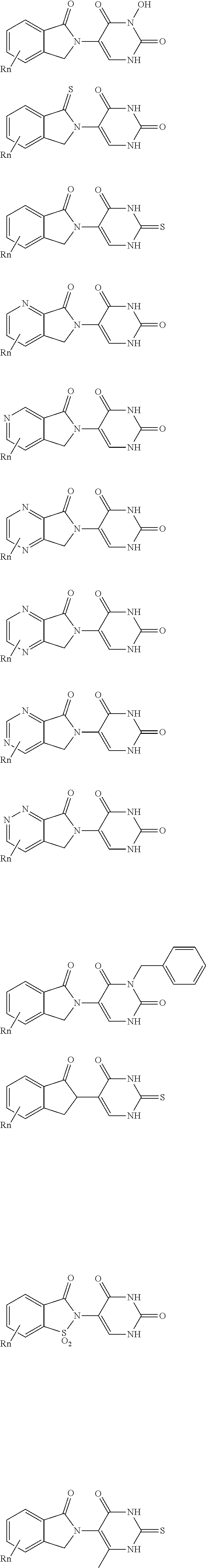

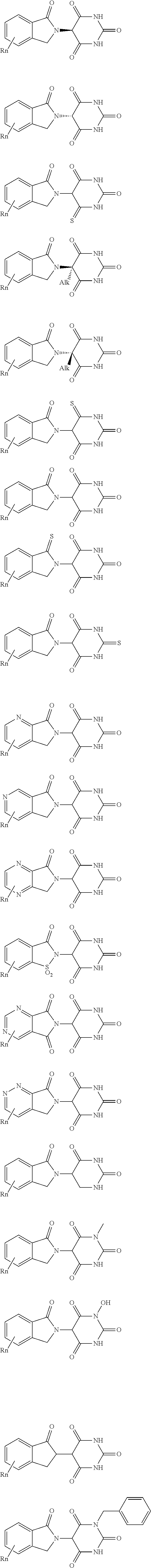

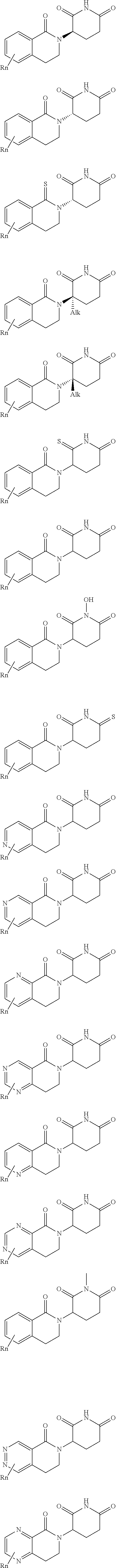

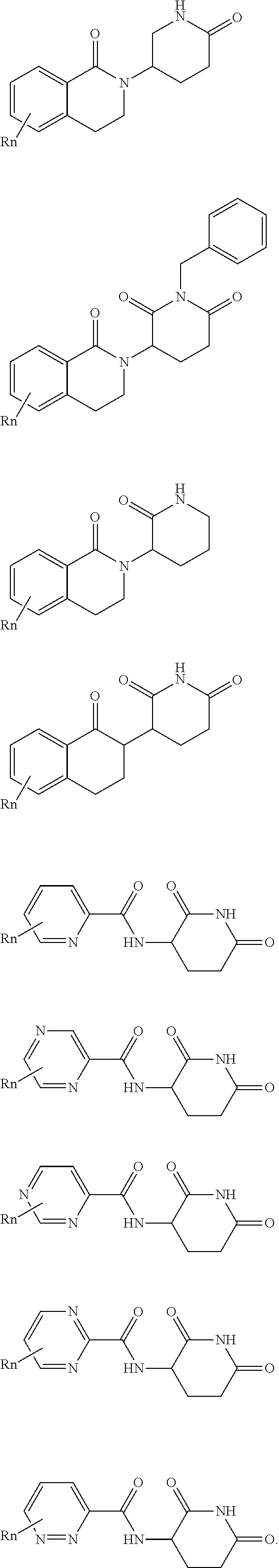

In any of the compounds described herein, the CLM comprises a chemical structure selected from the group:

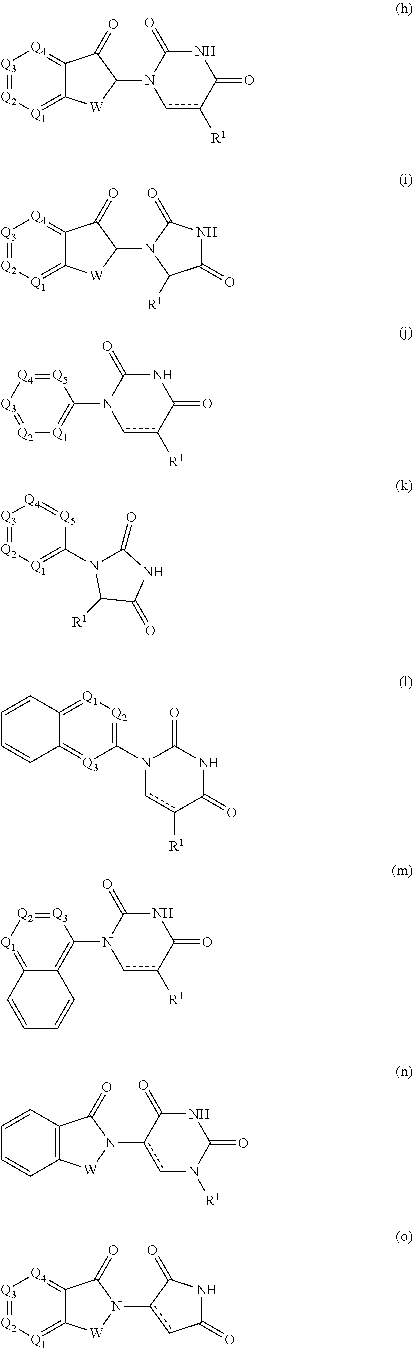

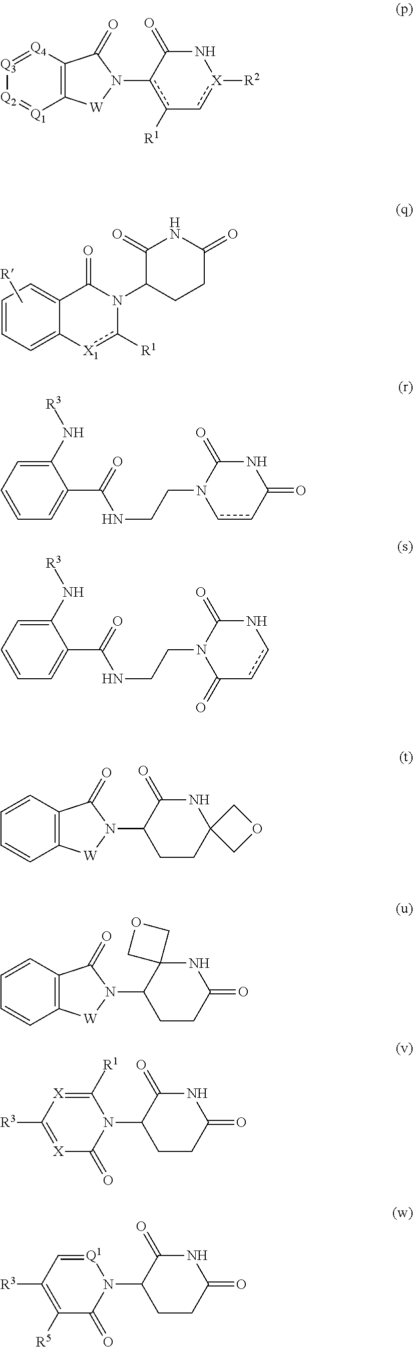

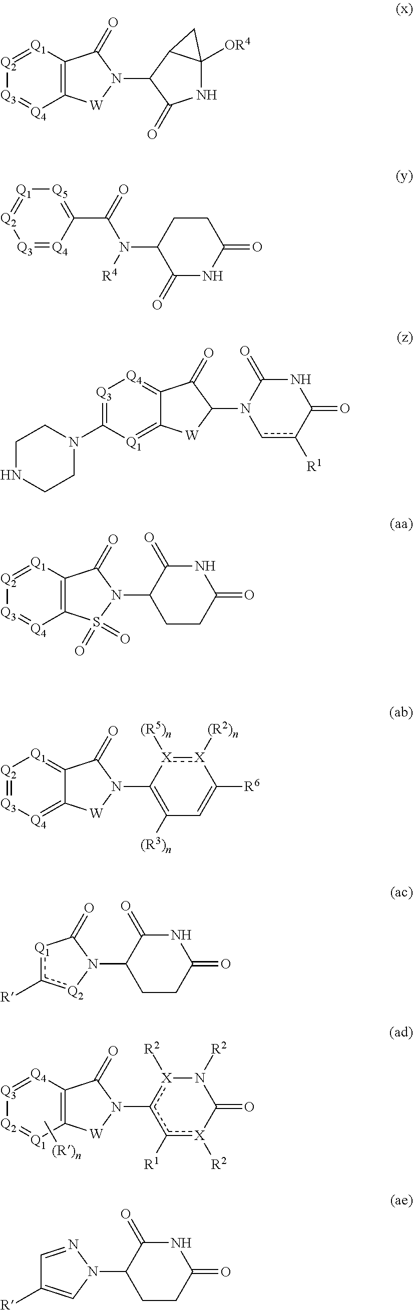

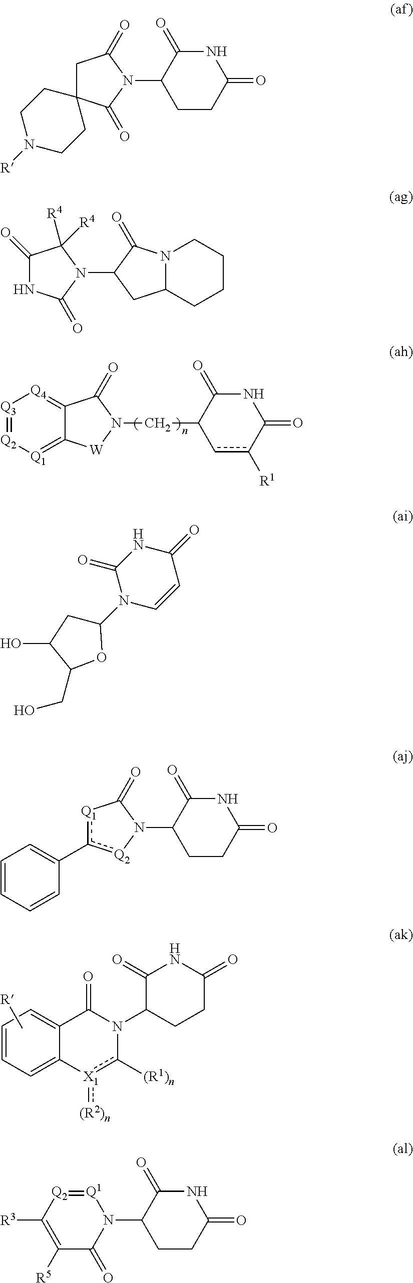

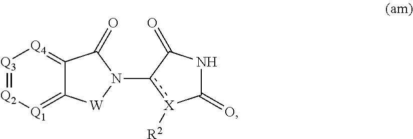

wherein:
W is independently selected from CH₂, CHR, C═O, SO₂, NH, and N-alkyl;
Q₁, Q₂, Q₃, Q₄, Q₅ are each independently represent a carbon C or N substituted with a group independently selected from R′, N or N-oxide;
R¹ is selected from absent, H, OH, CN, C1-C3 alkyl, C═O;
R² is selected from the group absent, H, OH, CN, C1-C3 alkyl, CHF₂, CF₃, CHO, C(═O)NH₂;
R³ is selected from H, alkyl (e.g., C1-C6 or C1-C3 alkyl), substituted alkyl (e.g., substituted C1-C6 or C1-C3 alkyl), alkoxy (e.g., C1-C6 or C1-C3 alkoxyl), substituted alkoxy (e.g., substituted C1-C6 or C1-C3 alkoxyl);
R⁴ is selected from H, alkyl, substituted alkyl;
R⁵ and R⁶ are each independently H, halogen, C(═O)R′, CN, OH, CF₃;
X is C, CH, C═O, or N;
X₁ is C═O, N, CH, or CH₂;
R′ is selected from H, halogen, amine, alkyl (e.g., C1-C3 alkyl), substituted alkyl (e.g., substituted C1-C3 alkyl), alkoxy (e.g., C1-C3 alkoxyl), substituted alkoxy (e.g., substituted C1-C3 alkoxyl), NR²R³, C(═O)OR², optionally substituted phenyl;
n is 0-4;
[symbol] is a single or double bond; and
the CLM is covalently joined to a PTM, a chemical linker group (L), a ULM, CLM (or CLM′) or combination thereof.

In any aspect or embodiment described herein, the CLM or CLM′ is covalently joined to a PTM, a chemical linker group (L), a ULM, a CLM, a CLM′, or a combination thereof via an R group (such as, R, R¹, R², R³, R⁴ or R′), W, X, or a Q group (such as, Q₁, Q₂, Q₃, Q₄, or Q₅).

In any of the embodiments described herein, the CLM or CLM′ is covalently joined to a PTM, a chemical linker group (L), a ULM, a CLM, a CLM′, or a combination thereof via W, X, R, R¹, R², R³, R⁴, R⁵, R′, Q₁, Q₂, Q₃, Q₄, and Q₅.

In any of the embodiments described herein, the W, X, R¹, R², R³, R⁴, R′, Q₁, Q₂, Q₃, Q₄, and Q₅ can independently be covalently coupled to a linker and/or a linker to which is attached to one or more PTM, ULM, ULM′, CLM or CLM′ groups.

More specifically, non-limiting examples of CLMs include those shown below as well as “hybrid” molecules or compounds that arise from combining 1 or more features of the following compounds:

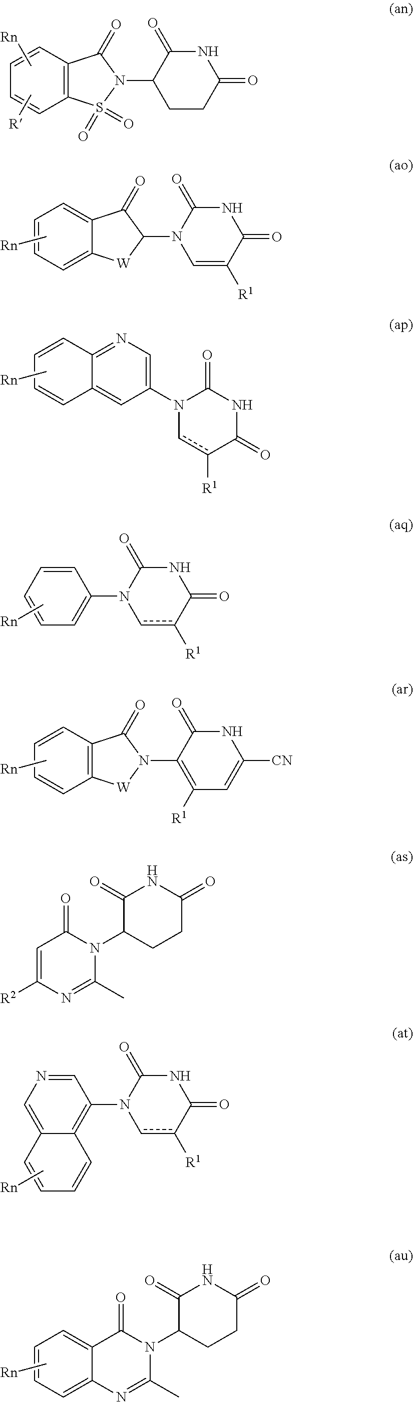

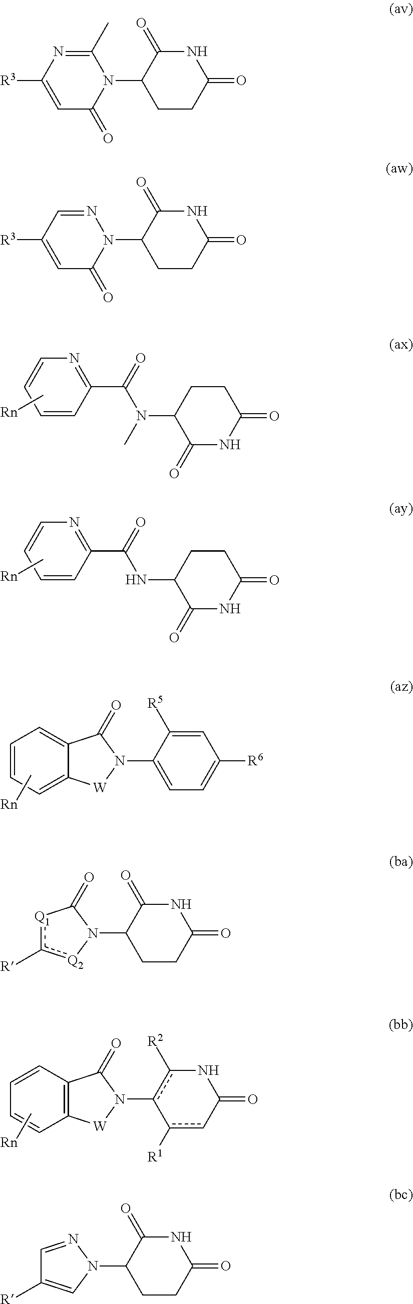

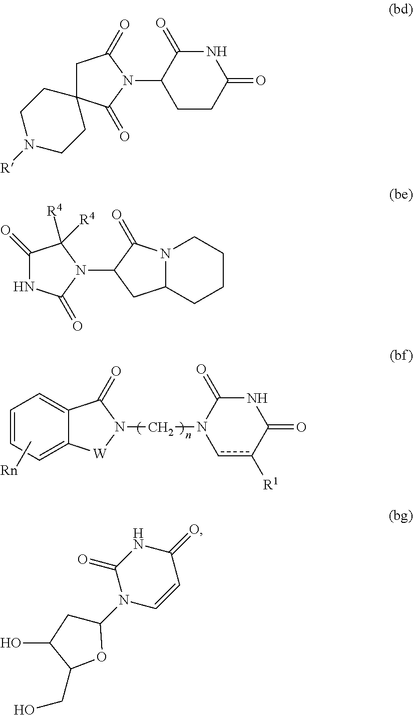

wherein:
W is independently selected from the group CH₂, CHR, C═O, SO₂, NH, and N-alkyl;
R¹ is selected from the group absent, H, CH, CN, C1-C3 alkyl;
R² is H or a C1-C3 alkyl;
R³ is selected from H, alkyl, substituted alkyl, alkoxy, substituted alkoxy;
R⁴ is methyl or ethyl;
R⁵ is H or halo;
R⁶ is H or halo;
R of the CLM is H;
R′ is H or an attachment point for a PTM, a PTM′, a chemical linker group (L), a ULM, a CLM, a CLM′,
Q₁ and Q₂ are each independently C or N substituted with a group independently selected from H or C1-C3 alkyl;
[symbol] is a single or double bond; and
Rn comprises a functional group or an atom.

In any of the embodiments described herein, the W, R¹, R², Q₁, Q₂, Q₃, Q₄, and Rn can independently be covalently coupled to a linker and/or a linker to which is attached one or more PTM, ULM, ULM′, CLM or CLM′ groups.

In any of the embodiments described herein, the R¹, R², Q₁, Q₂, Q₃, Q₄, and Rn can independently be covalently coupled to a linker and/or a linker to which is attached one or more PTM, ULM, ULM′, CLM or CLM′ groups.

In any of the embodiments described herein, the Q₁, Q₂, Q₃, Q₄, and Rn can independently be covalently coupled to a linker and/or a linker to which is attached one or more PTM, ULM, ULM′, CLM or CLM′ groups.

In any aspect or embodiment described herein, Rₙ is modified to be covalently joined to the linker group (L), a PTM, a ULM, a second CLM having the same chemical structure as the CLM, a CLM′, a second linker, or any multiple or combination thereof.

In any aspect or embodiment described herein, the CLM is selected from:

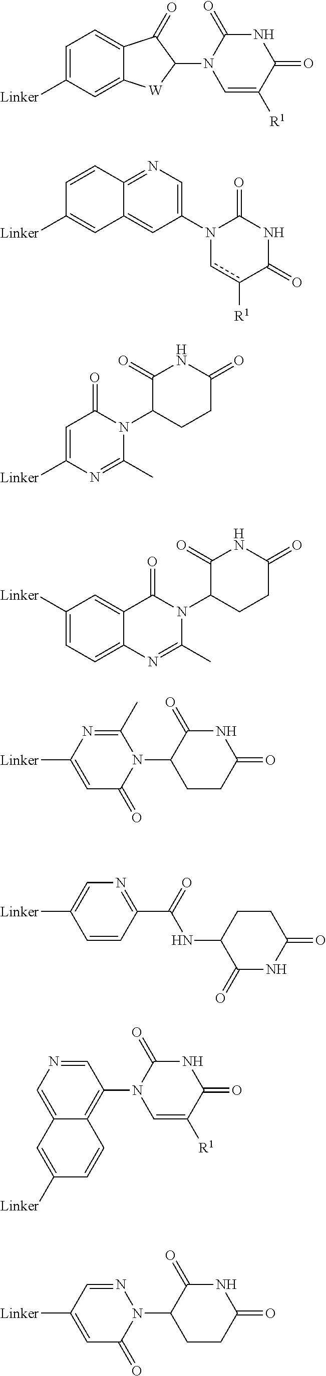

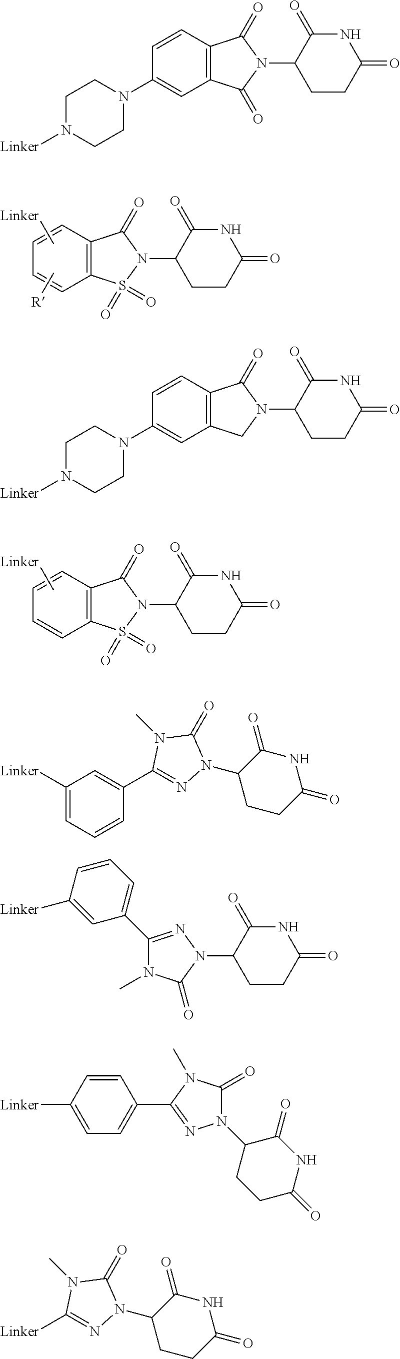

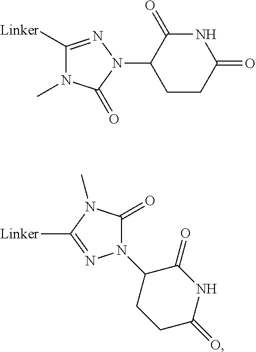

wherein R′ is a halogen and R¹ is as described in any aspect or embodiment described herein.

In certain cases, “CLM” can be imides that bind to cereblon E3 ligase. These imides and linker attachment point can be but not limited to the following structures:

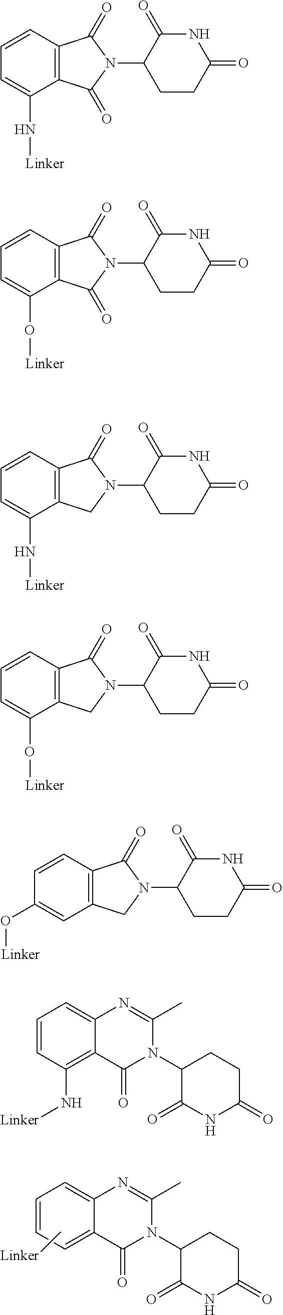

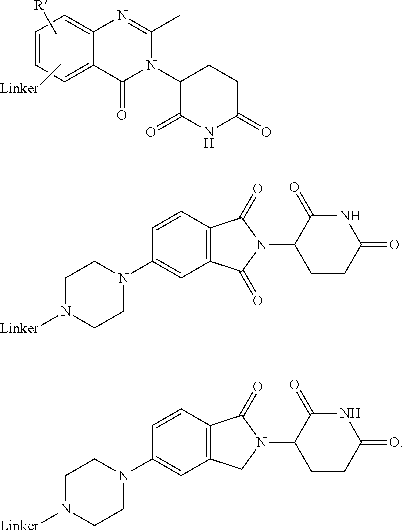

Exemplary VLMs

In certain embodiments of the compounds as described herein, ULM is VLM and comprises a chemical structure selected from the group ULM-a:

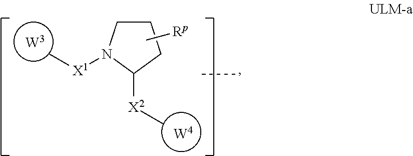

wherein
a dashed line indicates the attachment of at least one PTM, another ULM or VLM or MLM or ILM or CLM (i.e., ULM′ or VLM′ or CLM′ or ILM′ or MLM′), or a chemical linker moiety coupling at least one PTM, a ULM′ or a VLM′ or a CLM′ or a ILM′ or a MLM′ to the other end of the linker;
X¹, X² of Formula ULM-a are each independently selected from the group of a bond, O, NR^{Y3}, CR^{Y3}R^{Y4}, C═O, C═S, SO, and SO₂;
R^{Y3}, R^{Y4} of Formula ULM-a are each independently selected from the group of H, linear or branched C₁₋₆ alkyl, optionally substituted by 1 or more halo, optionally substituted C₁₋₆ alkoxyl (e.g., optionally substituted by 0-3 R^{P} groups);
R^{P} of Formula ULM-a is 0, 1, 2, or 3 groups, each independently selected from the group H, halo, —OH, C₁₋₃ alkyl, C═O;
W³ of Formula ULM-a is selected from the group of an optionally substituted T, an optionally substituted -T-N(R^{1a}R^{1b})X³, optionally substituted -T-N(R^{1a}R^{1b}), optionally substituted -T-Aryl, an optionally substituted -T-Heteroaryl, an optionally substituted T-biheteroaryl, an optionally substituted -T-Heterocycle, an optionally substituted -T-biheterocycle, an optionally substituted —NR¹-T-Aryl, an optionally substituted —NR¹-T-Heteroaryl or an optionally substituted —NR¹-T-Heterocycle;
X³ of Formula ULM-a is C═O, R¹, R^{1a}, R^{1b};
each of R¹, R^{1a}, R^{1b} is independently selected from the group consisting of H, linear or branched C₁-C₆ alkyl group optionally substituted by 1 or more halo or —OH groups, R^{Y3}C═O, R^{Y3}C═S, R^{Y3}SO, R^{Y3}SO₂, N(R^{Y3}R^{Y4})C═O, N(R^{Y3}R^{Y4)}C═S, N(R^{Y3}R^{Y4})SO, and N(R^{Y3}R^{Y4})SO₂;
T of Formula ULM-a is selected from the group of an optionally substituted alkyl, —(CH₂)ₙ-group, wherein each one of the methylene groups is optionally substituted with one or two substituents selected from the group of halogen, methyl, a linear or branched C₁-C₆ alkyl group optionally substituted by 1 or more halogen or —OH groups or an amino acid side chain optionally substituted;
W⁴ of Formula ULM-a is an optionally substituted —NR1-T-Aryl, wherein the aryl group may be optionally substituted with an optionally substituted 5-6 membered heteroaryl, an optionally substituted —NR1-T-Heteroaryl group or an optionally substituted —NR1-T-Heterocycle, where —NR1 is covalently bonded to X² and R¹ is H or CH₃, preferably H.

In any of the embodiments described herein, T is selected from the group of an optionally substituted alkyl, —(CH₂)ₙ— group, wherein each one of the methylene groups is optionally substituted with one or two substituents selected from the group of halogen, methyl, optionally substituted alkoxy, a linear or branched C₁-C₆ alkyl group optionally substituted by 1 or more halogen, C(O) NR¹R^{1a}, or NR¹R^{1a} or R¹ and R^{1a} are joined to form an optionally substituted heterocycle, or —OH groups or an amino acid side chain optionally substituted; and n is 0 to 6, often 0, 1, 2, or 3, preferably 0 or 1.

In certain embodiments, W⁴ of Formula ULM-a is

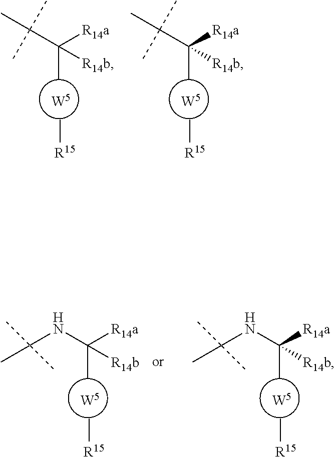

wherein R₁₄ₐ, R_{14b}, are each independently selected from the group of H, haloalkyl, or optionally substituted alkyl.

In any of the embodiments, W⁵ of Formula ULM-a is selected from the group of a phenyl or a 5-10 membered heteroaryl,

R₁₅ of Formula ULM-a is selected from the group of H, halogen, CN, OH, NO₂, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐ SO₂R_{14b}, optionally substituted alkyl, optionally substituted haloalkyl, optionally substituted haloalkoxy; optionally substituted aryl; optionally substituted heteroaryl; optionally substituted cycloalkyl; or optionally substituted cycloheteroalkyl;

In additional embodiments, W⁴ substituents for use in the present disclosure also include specifically (and without limitation to the specific compound disclosed) the W⁴ substituents which are found in the identified compounds disclosed herein. Each of these W⁴ substituents may be used in conjunction with any number of W³ substituents which are also disclosed herein.

In certain additional embodiments, ULM-a, is optionally substituted by 0-3 R^{P} groups in the pyrrolidine moiety. Each R^{P} is independently H, halo, —OH, C1-3alkyl, C═O.

In any of the embodiments described herein, the W³, W⁴ of Formula ULM-a can independently be covalently coupled to a linker which is attached one or more PTM groups.

and wherein the dashed line indicates the site of attachment of at least one PTM, another ULM (ULM′) or a chemical linker moiety coupling at least one PTM or a ULM′ or both to ULM.

In certain embodiments, ULM is VHL and is represented by the structure:

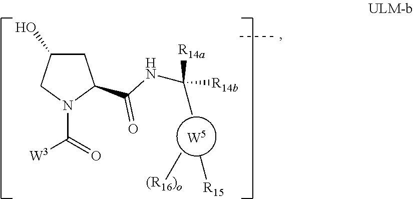

wherein:
W³ of Formula ULM-b is selected from the group of an optionally substituted aryl, optionally substituted heteroaryl, or

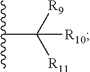

R₉ and R₁₀ of Formula ULM-b are independently hydrogen, optionally substituted alkyl, optionally substituted cycloalkyl, optionally substituted hydroxyalkyl, optionally substituted heteroaryl, or haloalkyl, or R₉, R₁₀, and the carbon atom to which they are attached form an optionally substituted cycloalkyl;
R₁₁ of Formula ULM-b is selected from the group of an optionally substituted heterocyclic, optionally substituted alkoxy, optionally substituted heteroaryl, optionally substituted aryl,

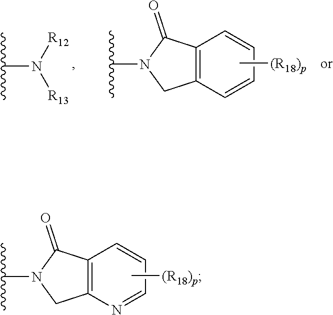

R₁₂ of Formula ULM-b is selected from the group of H or optionally substituted alkyl;
R₁₃ of Formula ULM-b is selected from the group of H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R₁₄ₐ, R_{14b} of Formula ULM-b, are each independently selected from the group of H, haloalkyl, or optionally substituted alkyl;
W⁵ of Formula ULM-b is selected from the group of a phenyl or a 5-10 membered heteroaryl,
R₁₅ of Formula ULM-b is selected from the group of H, halogen, CN, OH, NO₂, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐSO₂R_{14b}, optionally substituted alkyl, optionally substituted haloalkyl, optionally substituted haloalkoxy; optionally substituted aryl; optionally substituted heteroaryl; optionally substituted cycloalkyl; or optionally substituted cycloheteroalkyl;
R₁₆ of Formula ULM-b is independently selected from the group of halo, optionally substituted alkyl, optionally substituted haloalkyl, hydroxy, or optionally substituted haloalkoxy;
o of Formula ULM-b is 0, 1, 2, 3, or 4;
R₁₈ of Formula ULM-b is independently selected from the group of H, halo, optionally substituted alkoxy, cyano, optionally substituted alkyl, haloalkyl, haloalkoxy or a linker; and
p of Formula ULM-b is 0, 1, 2, 3, or 4, and wherein the dashed line indicates the site of attachment of at least one PTM, another ULM (ULM′) or a chemical linker moiety coupling at least one PTM or a ULM′ or both to ULM.

In certain embodiments, R₁₅ of Formula ULM-b is

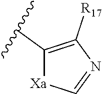

wherein R₁₇ is H, halo, optionally substituted C₃₋₆cycloalkyl, optionally substituted C₁₋₆alkyl, optionally substituted C₁₋₆alkenyl, and C₁₋₆haloalkyl; and Xa is S or O.

In certain embodiments, R₁₇ of Formula ULM-b is selected from the group methyl, ethyl, isopropyl, and cyclopropyl.

In certain additional embodiments, R₁₅ of Formula ULM-b is selected from the group consisting of:

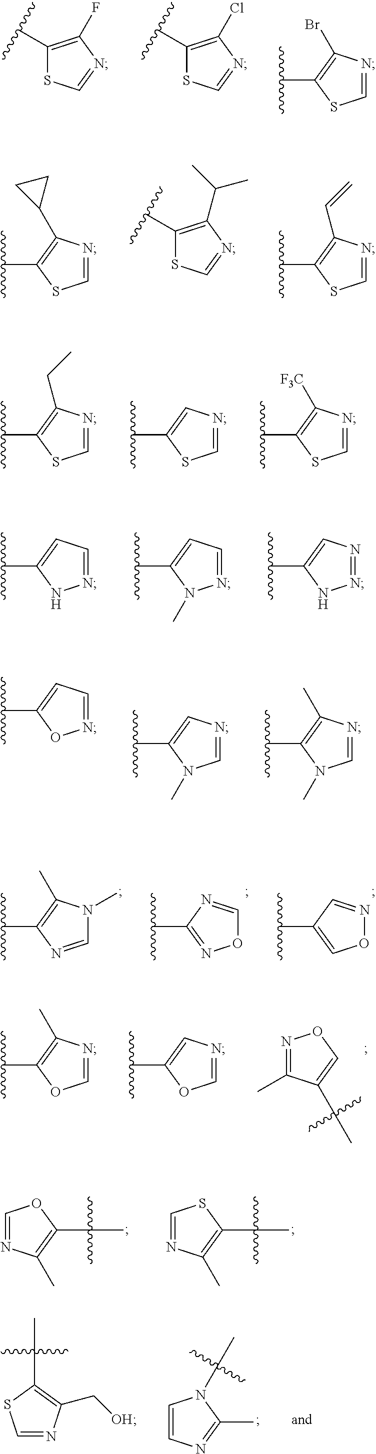

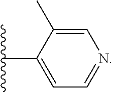 [SMILES: *c1ccncc1C]

In certain embodiments, R₁₁ of Formula ULM-b is selected from the group consisting of:

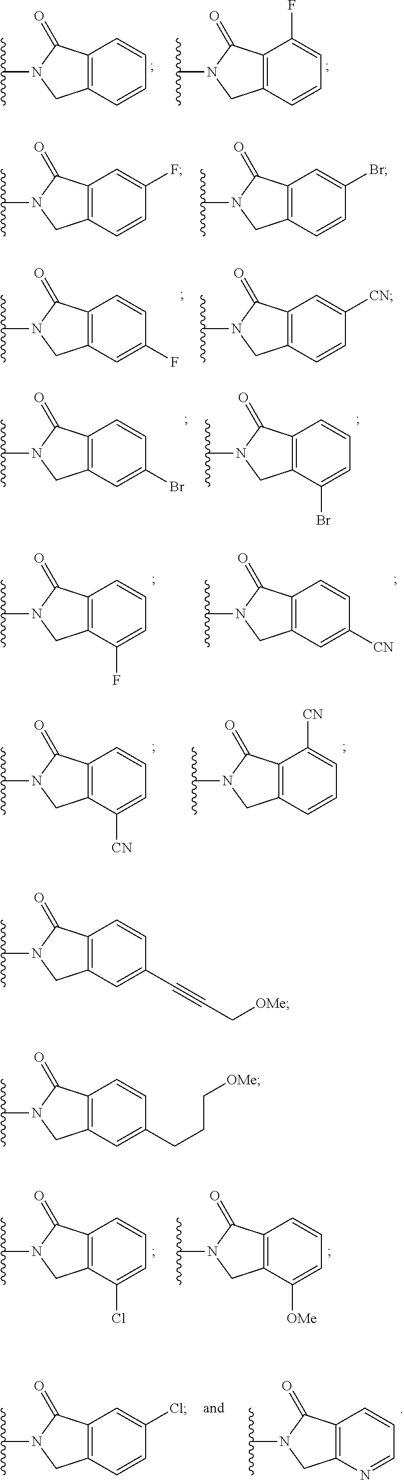

In certain embodiments, ULM has a chemical structure selected from the group of:

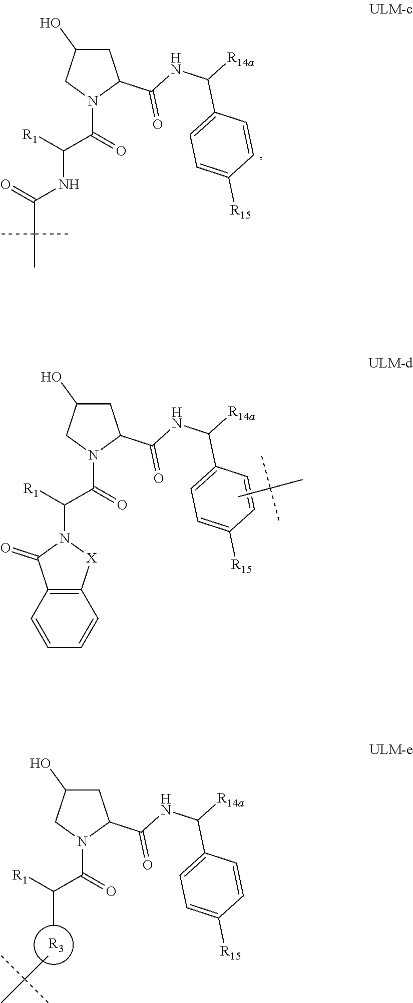

wherein:
R₁ of Formulas ULM-c, ULM-d, and ULM-e is H, ethyl, isopropyl, tert-butyl, sec-butyl, cyclopropyl, cyclobutyl, cyclopentyl, or cyclohexyl; optionally substituted alkyl, optionally substituted hydroxyalkyl, optionally substituted heteroaryl, or haloalkyl;
R₁₄ₐ of Formulas ULM-c, ULM-d, and ULM-e is H, haloalkyl, optionally substituted alkyl, methyl, fluoromethyl, hydroxymethyl, ethyl, isopropyl, or cyclopropyl;
R₁₅ of Formulas ULM-c, ULM-d, and ULM-e is selected from the group consisting of H, halogen, CN, OH, NO₂, optionally substituted heteroaryl, optionally substituted aryl; optionally substituted alkyl, optionally substituted haloalkyl, optionally substituted haloalkoxy, optionally substituted cycloalkyl, or optionally substituted cycloheteroalkyl;
X of Formulas ULM-c, ULM-d, and ULM-e is C, CH₂, or C═O
R₃ of Formulas ULM-c, ULM-d, and ULM-e is absent or an optionally substituted 5 or 6 membered heteroaryl; and
wherein the dashed line indicates the site of attachment of at least one PTM, another ULM (ULM′) or a chemical linker moiety coupling at least one PTM or a ULM′ or both to ULM.

In certain embodiments, ULM comprises a group according to the chemical structure:

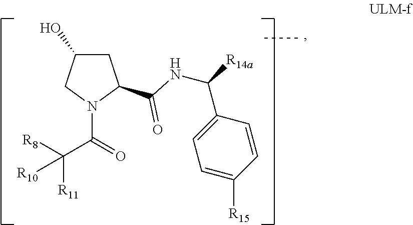

wherein:
R₁₄ₐ of Formula ULM-f is H, haloalkyl, optionally substituted alkyl, methyl, fluoromethyl, hydroxymethyl, ethyl, isopropyl, or cyclopropyl;
R₉ of Formula ULM-f is H;
R₁₀ of Formula ULM-f is H, ethyl, isopropyl, tert-butyl, sec-butyl, cyclopropyl, cyclobutyl, cyclopentyl, or cyclohexyl;
R₁₁ of Formula ULM-f is

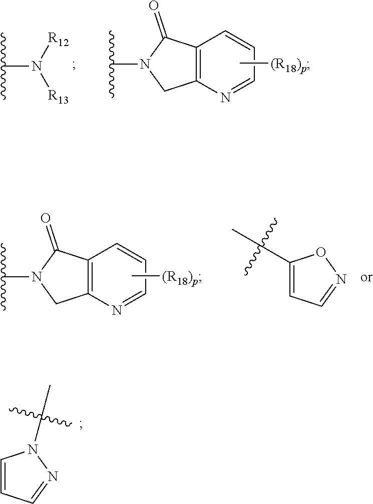

or optionally substituted heteroaryl;
p of Formula ULM-f is 0, 1, 2, 3, or 4;
each R₁₈ of Formula ULM-f is independently halo, optionally substituted alkoxy, cyano, optionally substituted alkyl, haloalkyl, haloalkoxy or a linker;
R₁₂ of Formula ULM-f is H, C═O;
R₁₃ of Formula ULM-f is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl,
R₁₅ of Formula ULM-f is selected from the group consisting of H, halogen, Cl, CN, OH, NO₂, optionally substituted heteroaryl, optionally substituted aryl;

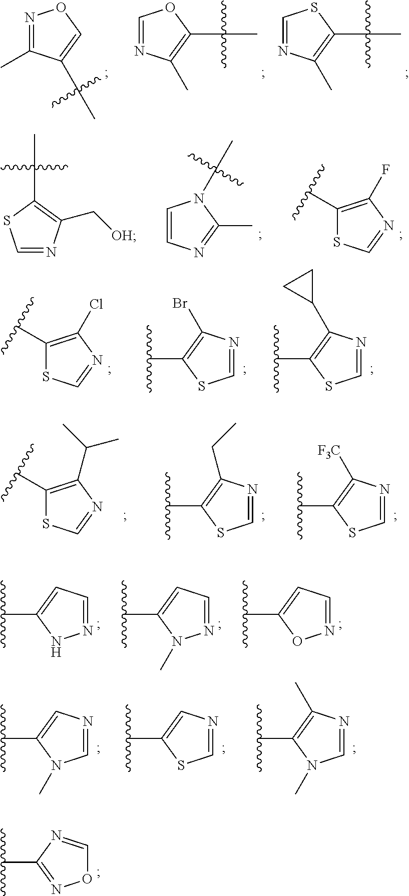

and
wherein the dashed line of Formula ULM-f indicates the site of attachment of at least one PTM, another ULM (ULM′) or a chemical linker moiety coupling at least one PTM or a ULM′ or both to ULM.

In certain embodiments, the ULM is selected from the following structures:

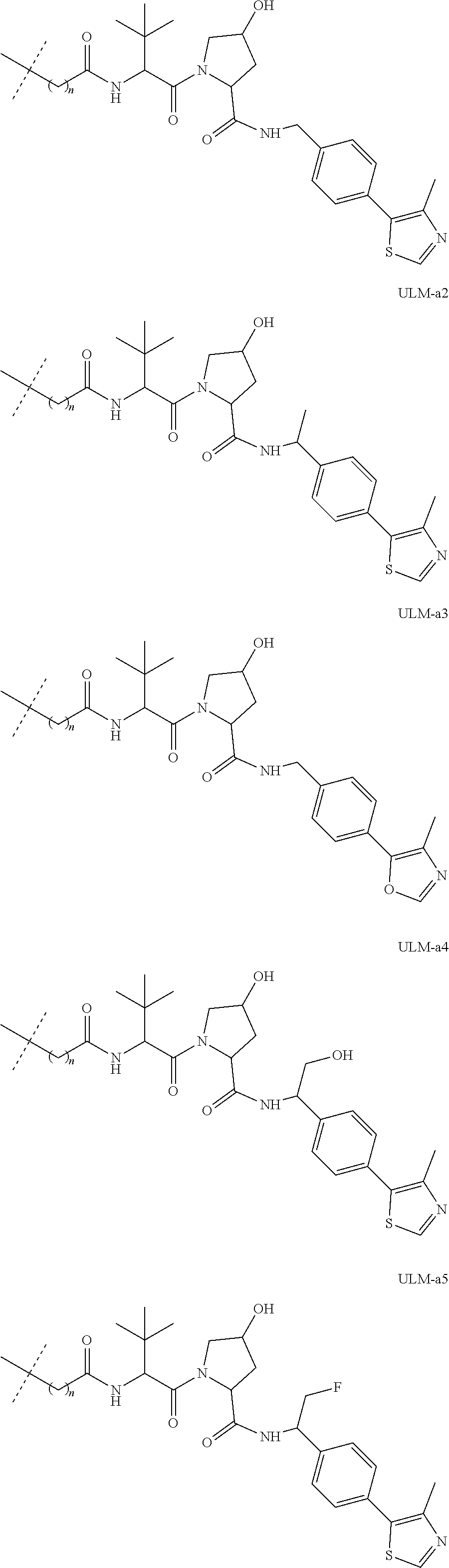

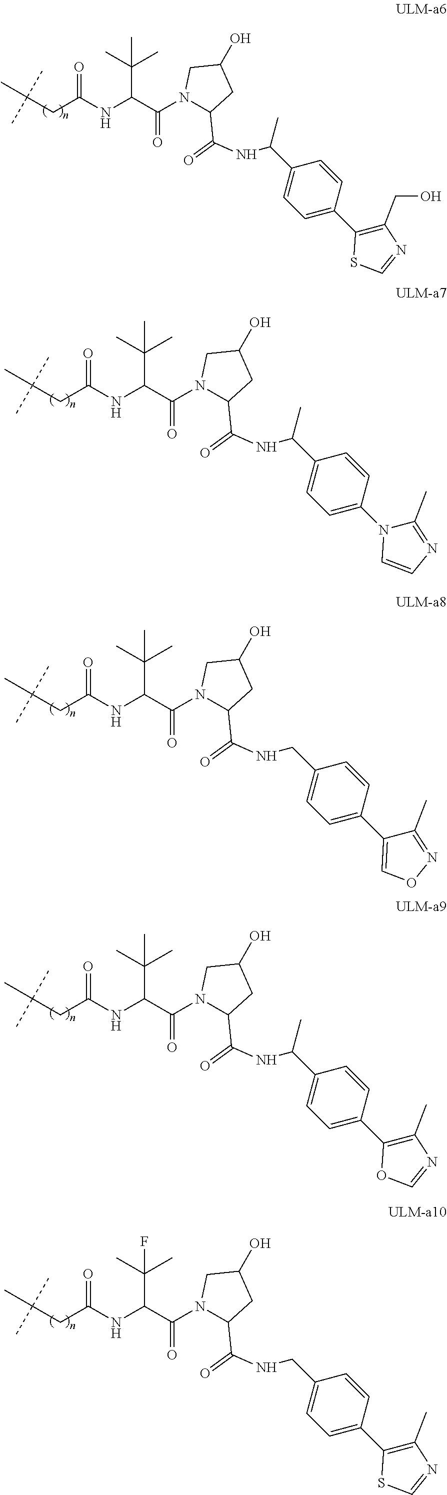

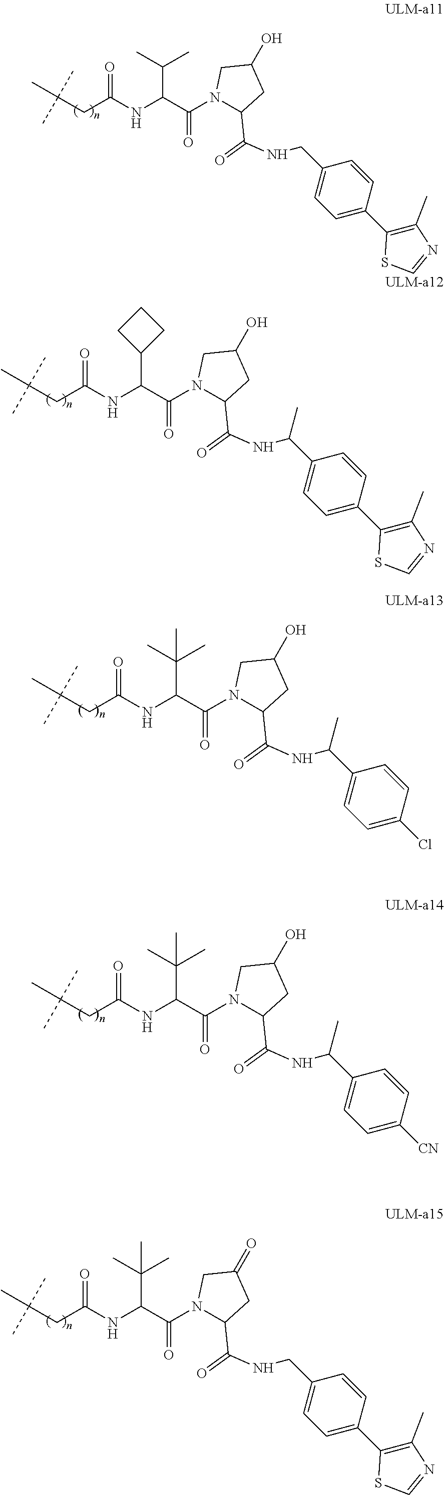

wherein n is 0 or 1.

In certain embodiments, the ULM is selected from the following structures:

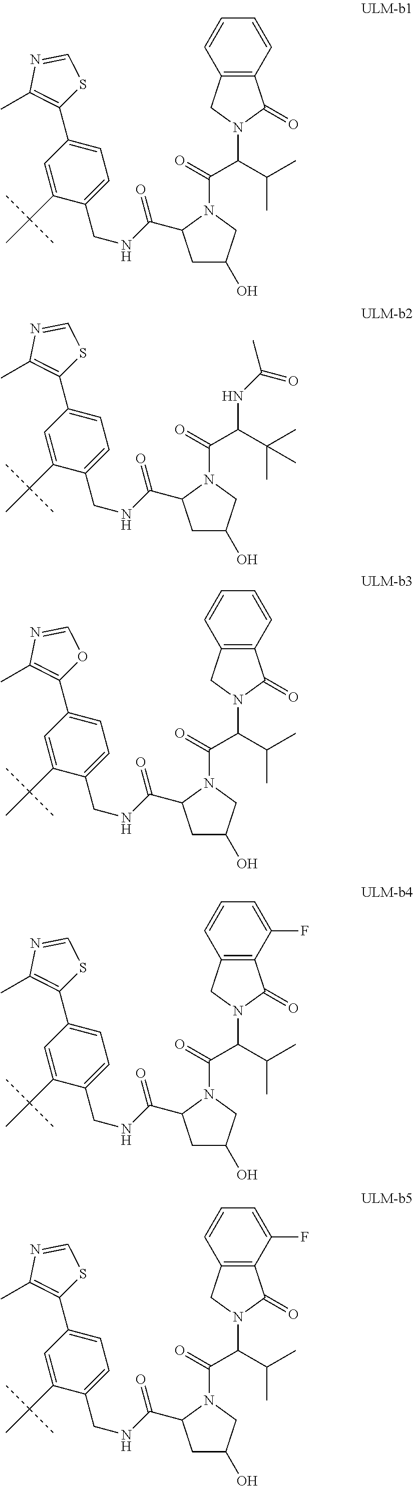

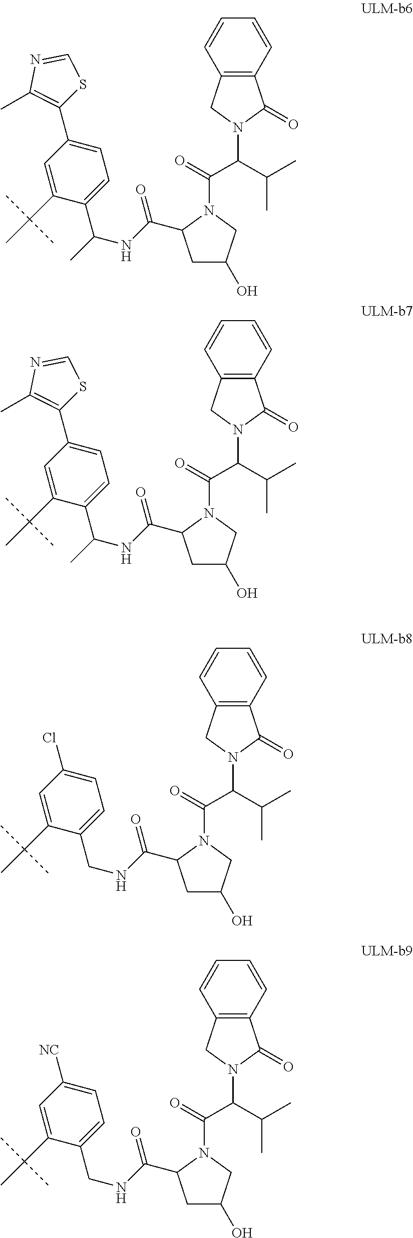

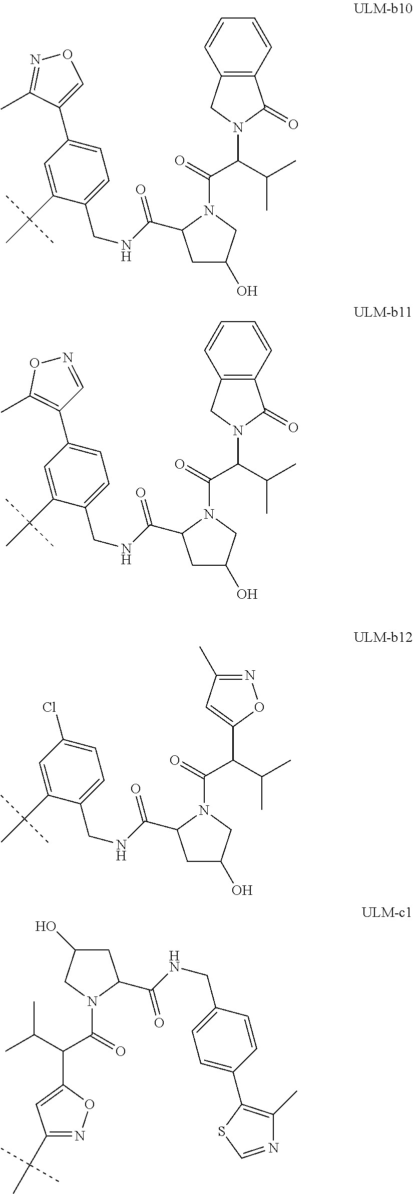

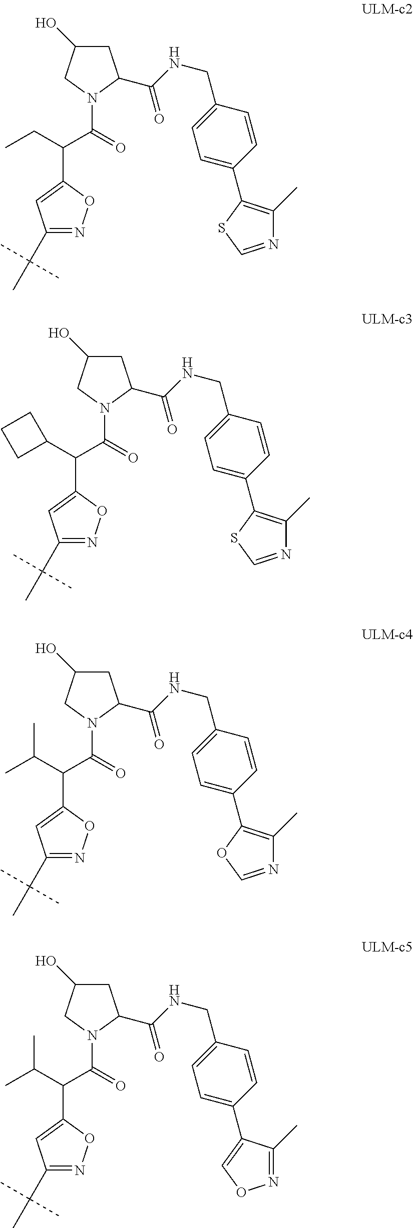

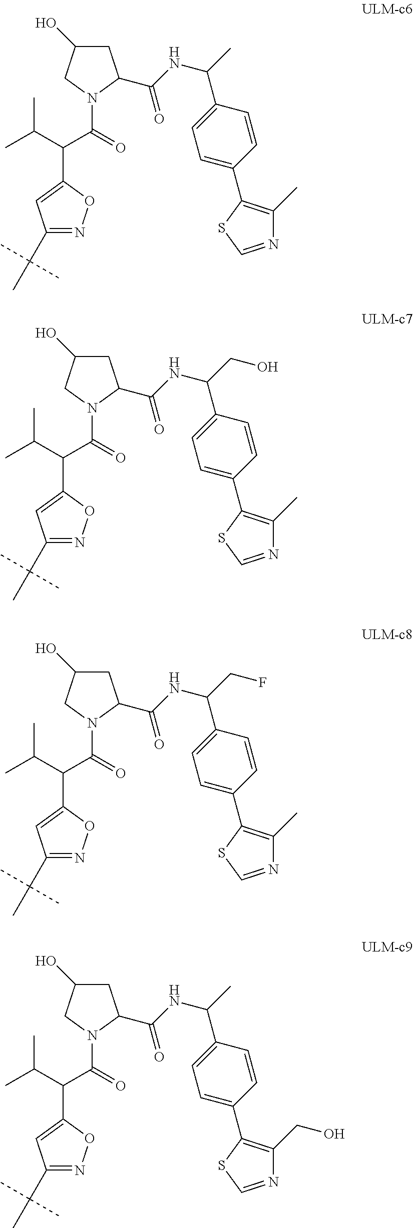

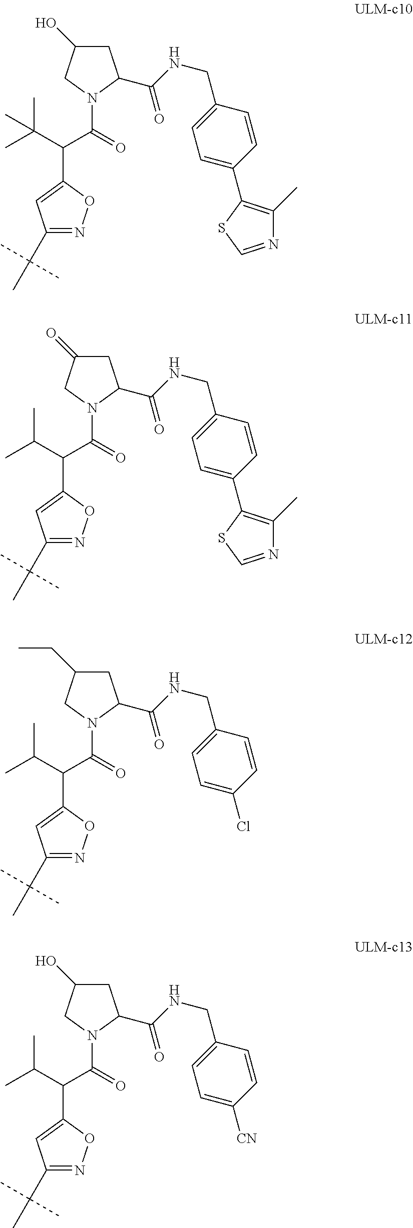

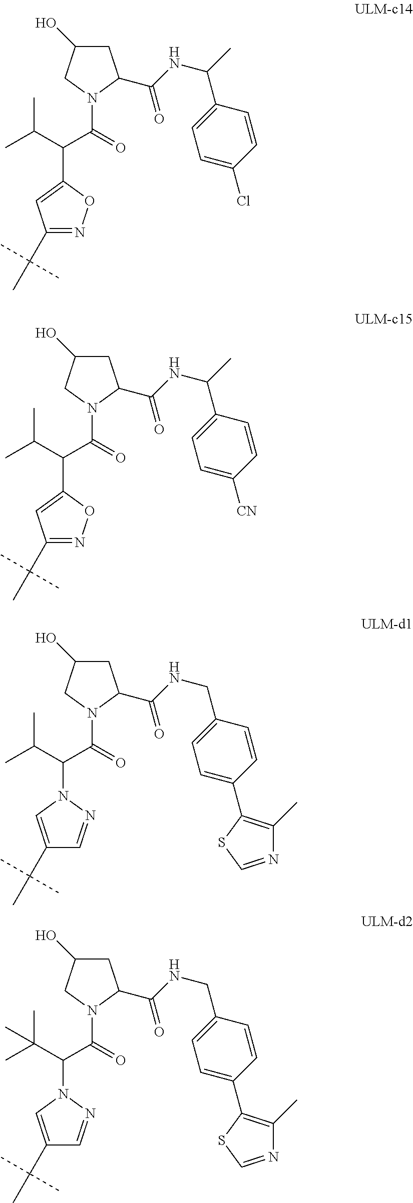

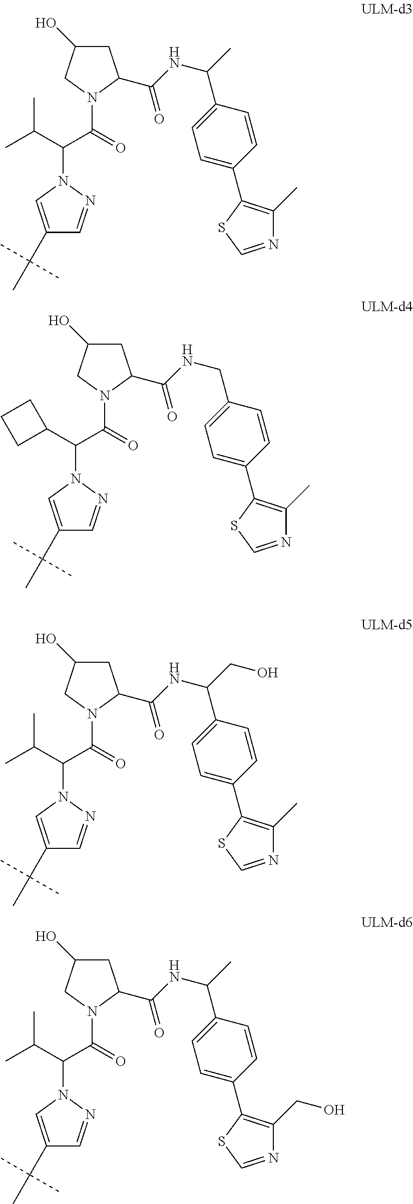

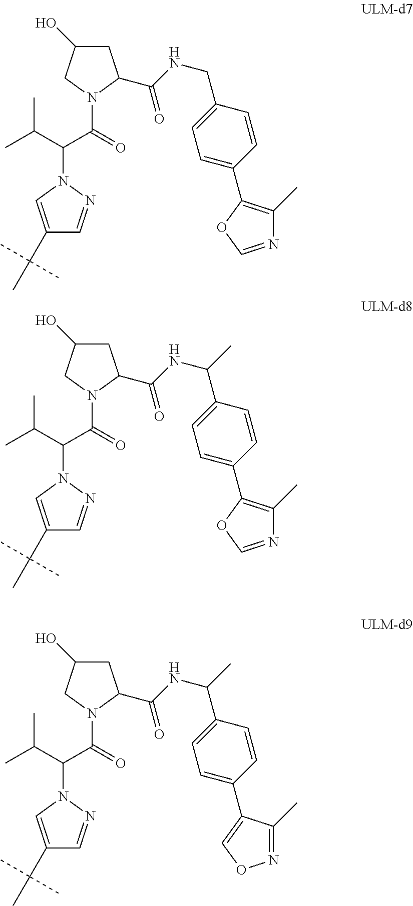

wherein, the phenyl ring in ULM-al through ULM-a15, ULM-b1 through ULM-b12, ULM-c1 through ULM-c15 and ULM-dl through ULM-d9 is optionally substituted with fluorine, lower alkyl and alkoxy groups, and wherein the dashed line indicates the site of attachment of at least one PTM, another ULM (ULM′) or a chemical linker moiety coupling at least one PTM or a ULM′ or both to ULM-a.

In one embodiment, the phenyl ring in ULM-a1 through ULM-a15, ULM-b1 through ULM-b12, ULM-c1 through ULM-c15 and ULM-dl through ULM-d9 can be functionalized as the ester to make it a part of the prodrug.

In certain embodiments, the hydroxyl group on the pyrrolidine ring of ULM-al through ULM-a15, ULM-b1 through ULM-b12, ULM-c1 through ULM-c15 and ULM-d1 through ULM-d9, respectively, comprises an ester-linked prodrug moiety.

In any of the aspects or embodiments described herein, the ULM and where present, ULM′, are each independently a group according to the chemical structure:

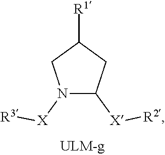

wherein:
R^{1′} of ULM-g is an optionally substituted C₁-C₆ alkyl group, an optionally substituted —(CH₂)ₙOH, an optionally substituted —(CH₂)ₙSH, an optionally substituted (CH₂)ₙ—O—(C₁-C₆)alkyl group, an optionally substituted (CH₂)ₙ—WCOCW—(C₀-C₆)alkyl group containing an epoxide moiety WCOCW where each W is independently H or a C₁-C₃ alkyl group, an optionally substituted —(CH₂)ₙCOOH, an optionally substituted —(CH₂)ₙC(O)—(C₁-C₆ alkyl), an optionally substituted —(CH₂)ₙNHC(O)—R₁, an optionally substituted —(CH₂)ₙC(O)—NR₁R₂, an optionally substituted —(CH₂)ₙOC(O)—NR₁R₂, —(CH₂O)ₙH, an optionally substituted —(CH₂)ₙOC(O)—(C₁-C₆ alkyl), an optionally substituted —(CH₂)ₙC(O)—O—(C₁-C₆ alkyl), an optionally substituted —(CH₂O)ₙCOOH, an optionally substituted —(OCH₂)ₙO—(C₁-C₆ alkyl), an optionally substituted —(CH₂O)ₙC(O)—(C₁-C₆ alkyl), an optionally substituted —(OCH₂)ₙNHC(O)—R₁, an optionally substituted —(CH₂O)ₙC(O)—NR₁R₂, —(CH₂CH₂O)ₙH, an optionally substituted —(CH₂CH₂O)ₙCOOH, an optionally substituted —(OCH₂CH₂)ₙO—(C₁-C₆ alkyl), an optionally substituted —(CH₂CH₂O)ₙC(O)—(C₁-C₆ alkyl), an optionally substituted —(OCH₂CH₂)ₙNHC(O)—R₁, an optionally substituted —(CH₂CH₂O)ₙC(O)—NR₁R₂, an optionally substituted —SO₂R_{S}, an optionally substituted S(O)R_{S}, NO₂, CN or halogen (F, Cl, Br, I, preferably F or Cl);
R₁ and R₂ of ULM-g are each independently H or a C₁-C₆ alkyl group which may be optionally substituted with one or two hydroxyl groups or up to three halogen groups (preferably fluorine);
R_{S} of ULM-g is a C₁-C₆ alkyl group, an optionally substituted aryl, heteroaryl or heterocycle group or a —(CH₂)ₘNR₁R₂ group;
X and X′ of ULM-g are each independently C═O, C═S, —S(O), S(O)₂, (preferably X and X′ are both C═O);
R^{2′} of ULM-g is an optionally substituted —(CH₂)ₙ—(C═O)ᵤ(NR₁)ᵥ(SO₂)_{w}alkyl group, an optionally substituted —(CH₂)ₙ—(C═O)ᵤ(NR₁)ᵥ(SO₂)_{w}NR_{1N}R_{2N} group, an optionally substituted —(CH₂)ₙ—(C═O)ᵤ(NR₁)ᵥ(SO₂)_{w}-Aryl, an optionally substituted —(CH₂)ₙ—(C═O)ᵤ(NR₁)ᵥ(SO₂)_{w}-Heteroaryl, an optionally substituted —(CH₂)ₙ—(C═O)NR₁(SO₂)_{w}-Heterocycle, an optionally substituted —NR¹—(CH₂)ₙ—C(O)(NR₁)(SO₂)_{w}-alkyl, an optionally substituted —NR¹—(CH₂)ₙ—C(O)(NR₁)(SO₂)_{w}—NR_{1N}R_{2N}, an optionally substituted —NR¹—(CH₂)ₙ—C(O)(NR₁)(SO₂)_{w}—NR₁C(O)R_{1N}, an optionally substituted —NR¹—(CH₂)ₙ—(C═O)(NR₁)(SO₂)_{w}-Aryl, an optionally substituted —NR¹—(CH₂)ₙ—(C═O)ᵤ(NR₁)ᵥ(SO₂)_{w}-Heteroaryl or an optionally substituted —NR¹—(CH₂)ₙ—(C═O)ᵥNR₁(SO₂)_{w}-Heterocycle, an optionally substituted —X^{R2′}-alkyl group; an optionally substituted —X^{R2′}— Aryl group; an optionally substituted —X^{R2′}— Heteroaryl group; an optionally substituted —X^{R2′}— Heterocycle group; an optionally substituted;
R^{3′} of ULM-g is an optionally substituted alkyl, an optionally substituted —(CH₂)ₙ—(O)ᵤ(NR₁)ᵥ(SO₂)_{w}-alkyl, an optionally substituted —(CH₂)ₙ—C(O)ᵤ(NR₁)ᵥ(SO₂)_{w}—NR_{1N}R_{2N}, an optionally substituted —(CH₂)ₙ—C(O)ᵤ(NR₁)ᵥ(SO₂)_{w}—NR₁C(O)R_{1N}, an optionally substituted —(CH₂)ₙ—C(O)(NR₁)ᵥ(SO₂)_{w}—C(O)NR₁R₂, an optionally substituted —(CH₂)ₙ—C(O)ᵤ(NR₁)ᵥ(SO₂)_{w}-Aryl, an optionally substituted —(CH₂)ₙ—C(O)ᵤ(NR₁)(SO₂)_{w}-Heteroaryl, an optionally substituted —(CH₂)ₙ—C(O)ᵤ(NR₁)ᵥ(SO₂)_{w}-Heterocycle, an optionally substituted —NR¹—(CH₂)ₙ—C(O)ᵤ(NR₁)ᵥ(SO₂)_{w}-alkyl, an optionally substituted —NR¹—(CH₂)ₙ—C(O)ᵤ(NR₁)ᵥ(SO₂)_{w}—NR_{1N}R_{2N}, an optionally substituted —NR¹—(CH₂)ₙ—C(O)ᵤ(NR₁)ᵥ(SO₂)_{w}—NR₁C(O)R_{1N}, an optionally substituted —NR¹—(CH₂)ₙ—C(O)ᵤ(NR₁)ᵥ(SO₂)_{w}-Aryl, an optionally substituted —NR¹—(CH₂)ₙ—C(O)ᵤ(NR₁)ᵥ(SO₂)_{w}-Heteroaryl, an optionally substituted —NR¹—(CH₂)ₙ—C(O)ᵤ(NR₁)ᵥ(SO₂)_{w}-Heterocycle, an optionally substituted —O—(CH₂)n-(C═O)ᵤ(NR₁)ᵥ(SO₂)_{w}-alkyl, an optionally substituted —O—(CH₂)n-(C═O)ᵤ(NR)ᵥ(SO₂)_{w}—NR_{1N}R_{2N}, an optionally substituted —O—(CH₂)n-(C═O)ᵤ(NR₁)ᵥ(SO₂)_{w}—NR₁C(O)R_{1N}, an optionally substituted —O—(CH₂)n-(C═O)ᵤ(NR₁)ᵥ(SO₂)_{w}-Aryl, an optionally substituted —O—(CH₂)ₙ—(C═O)ᵤ(NR₁)ᵥ(SO₂)_{w}-Heteroaryl or an optionally substituted —O—(CH₂)ₙ—(C═O)ᵤ(NR₁)ᵥ(SO₂)_{w}-Heterocycle; —(CH₂)ₙ—(V)_{n′}—(CH₂)ₙ—(V)_{n′}-alkyl group, an optionally substituted —(CH₂)ₙ—(V)_{n′}—(CH₂)ₙ—(V)_{n′}-Aryl group, an optionally substituted —(CH₂)ₙ—(V)_{n′}—(CH₂)ₙ—(V)_{n′}—Heteroaryl group, an optionally substituted —(CH₂)ₙ—(V)_{n′}—(CH₂)ₙ—(V)_{n′}-Heterocycle group, an optionally substituted —(CH₂)ₙ—N(R_{1′})(C═O)_{m′}—(V)_{n′}-alkyl group, an optionally substituted —(CH₂)ₙ—N(R_{1′})(C═O)_{m′}—(V)_{n′}-Aryl group, an optionally substituted —(CH₂)ₙ—N(R_{1′})(C═O)_{m′}—(V)_{n′}-Heteroaryl group, an optionally substituted —(CH₂)ₙ—N(R_{1′})(C═O)_{m′}—(V)_{n′}-Heterocycle group, an optionally substituted —X^{R3′}-alkyl group; an optionally substituted —X^{R3′}-Aryl group; an optionally substituted —X^{R3′}-Heteroaryl group; an optionally substituted —X^{R3′}-Heterocycle group; an optionally substituted;
R_{1N} and R_{2N} of ULM-g are each independently H, C₁-C₆ alkyl which is optionally substituted with one or two hydroxyl groups and up to three halogen groups or an optionally substituted —(CH₂)ₙ-Aryl, —(CH₂)ₙ-Heteroaryl or —(CH₂)ₙ-Heterocycle group;
V of ULM-g is O, S or NR₁;
R₁ of ULM-g is the same as above;
R¹ and R_{1′} of ULM-g are each independently H or a C₁-C₃ alkyl group;
X^{R2′} and X^{R3′} of ULM-g are each independently an optionally substituted —CH₂)ₙ—, —CH₂)ₙ—CH(Xᵥ)═CH(Xᵥ)— (cis or trans), —CH₂)ₙ—CH≡CH—, —(CH₂CH₂O)ₙ— or a C₃-C₆ cycloalkyl group, where Xᵥ is H, a halo or a C₁-C₃ alkyl group which is optionally substituted;
each m of ULM-g is independently 0, 1, 2, 3, 4, 5, 6;
each m′ of ULM-g is independently 0 or 1;
each n of ULM-g is independently 0, 1, 2, 3, 4, 5, 6;
each n′ of ULM-g is independently 0 or 1;
each u of ULM-g is independently 0 or 1;
each v of ULM-g is independently 0 or 1;
each w of ULM-g is independently 0 or 1; and
any one or more of R^{1′}, R^{2′}, R^{3′}, X and X′ of ULM-g is optionally modified to be covalently bonded to the PTM group through a linker group when PTM is not ULM′, or when PTM is ULM′, any one or more of R^{1′}, R^{2′}, R^{3′}, X and X′ of each of ULM and ULM′ are optionally modified to be covalently bonded to each other directly or through a linker group, or a pharmaceutically acceptable salt, stereoisomer, solvate or polymorph thereof.

In any of the aspects or embodiments described herein, the ULM and when present, ULM′, are each independently a group according to the chemical structure:

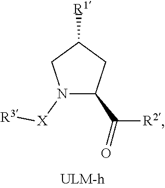

wherein:
each of R^{1′}, R^{2′} and R^{3′} of ULM-h are the same as above and X is C═O, C═S, —S(O) group or a S(O)₂ group, more preferably a C═O group, and
any one or more of R^{1′}, R^{2′} and R^{3′} of ULM-h are optionally modified to bind a linker group to which is further covalently bonded to the PTM group when PTM is not ULM′, or when PTM is ULM′, any one or more of R^{1′}, R^{2′}, R^{3′} of each of ULM and ULM′ are optionally modified to be covalently bonded to each other directly or through a linker group, or
a pharmaceutically acceptable salt, enantiomer, diastereomer, solvate or polymorph thereof.

In any of the aspects or embodiments described herein, the ULM, and when present, ULM′, are each independently according to the chemical structure:

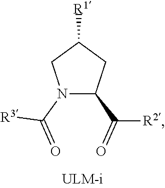

wherein:
any one or more of R^{1′}, R^{2′} and R^{3′} of ULM-I are optionally modified to bind a linker group to which is further covalently bonded to the PTM group when PTM is not ULM′, or when PTM is ULM′, any one or more of R^{1′}, R^{2′}, R^{3′} of each of ULM and ULM′ are optionally modified to be covalently bonded to each other directly or through a linker group, or
a pharmaceutically acceptable salt, enantiomer, diastereomer, solvate or polymorph thereof.

In further preferred aspects of the disclosure, R^{1′} of ULM-g through ULM-i is preferably a hydroxyl group or a group which may be metabolized to a hydroxyl or carboxylic group, such that the compound represents a prodrug form of an active compound. Exemplary preferred R^{1′} groups include, for example, —(CH₂)ₙOH, (CH₂)ₙ—O—(C₁-C₆)alkyl group, —(CH₂)ₙCOOH, —(CH₂O)ₙH, an optionally substituted —(CH₂)ₙOC(O)—(C₁-C₆ alkyl), or an optionally substituted —(CH₂)ₙC(O)—O—(C₁-C₆ alkyl), wherein n is 0 or 1. Where R^{1′} is or contains a carboxylic acid group, a hydroxyl group or an amine group, the hydroxyl group, carboxylic acid group or amine (each of which may be optionally substituted), may be further chemically modified to provide a covalent link to a linker group to which the PTM group (including a ULM′ group) is bonded;

X and X′, where present, of ULM-g and ULM-h are preferably a C═O, C═S, —S(O) group or a S(O)₂ group, more preferably a C═O group;

R^{2′} of ULM-g through ULM-i is preferably an optionally substituted —NR¹-T-Aryl, an optionally substituted —NR¹-T-Heteroaryl group or an optionally substituted —NR¹-T-Heterocycle, where R¹ is H or CH₃, preferably H and T is an optionally substituted —(CH₂)ₙ-group, wherein each one of the methylene groups may be optionally substituted with one or two substituents, preferably selected from halogen, an amino acid sidechain as otherwise described herein or a C₁-C₃ alkyl group, preferably one or two methyl groups, which may be optionally substituted; and n is 0 to 6, often 0, 1, 2 or 3, preferably 0 or 1. Alternatively, T may also be a —(CH₂O)ₙ— group, a —(OCH₂)ₙ— group, a —(CH₂CH₂O)ₙ— group, a —(OCH₂CH₂)ₙ— group, all of which groups are optionally substituted.

Preferred Aryl groups for R^{2′} of ULM-g through ULM-i include optionally substituted phenyl or naphthyl groups, preferably phenyl groups, wherein the phenyl or naphthyl group is connected to a PTM (including a ULM′ group) with a linker group and/or optionally substituted with a halogen (preferably F or Cl), an amine, monoalkyl- or dialkyl amine (preferably, dimethylamine), F, Cl, OH, COOH, C₁-C₆ alkyl, preferably CH₃, CF₃, OMe, OCF₃, NO₂, or CN group (each of which may be substituted in ortho-, meta- and/or para-positions of the phenyl ring, preferably para-), an optionally substituted phenyl group (the phenyl group itself is optionally connected to a PTM group, including a ULM′, with a linker group), and/or optionally substituted with at least one of F, Cl, OH, COOH, CH₃, CF₃, OMe, OCF₃, NO₂, or CN group (in ortho-, meta- and/or para-positions of the phenyl ring, preferably para-), a naphthyl group, which may be optionally substituted, an optionally substituted heteroaryl, preferably an optionally substituted isoxazole including a methylsubstituted isoxazole, an optionally substituted oxazole including a methylsubstituted oxazole, an optionally substituted thiazole including a methyl substituted thiazole, an optionally substituted isothiazole including a methyl substituted isothiazole, an optionally substituted pyrrole including a methylsubstituted pyrrole, an optionally substituted imidazole including a methylimidazole, an optionally substituted benzimidazole or methoxybenzylimidazole, an optionally substituted oximidazole or methyloximidazole, an optionally substituted diazole group, including a methyldiazole group, an optionally substituted triazole group, including a methylsubstituted triazole group, an optionally substituted pyridine group, including a halo- (preferably, F) or methylsubstitutedpyridine group or an oxapyridine group (where the pyridine group is linked to the phenyl group by an oxygen), an optionally substituted furan, an optionally substituted benzofuran, an optionally substituted dihydrobenzofuran, an optionally substituted indole, indolizine or azaindolizine (2, 3, or 4-azaindolizine), an optionally substituted quinoline, an optionally substituted group according to the chemical structure:

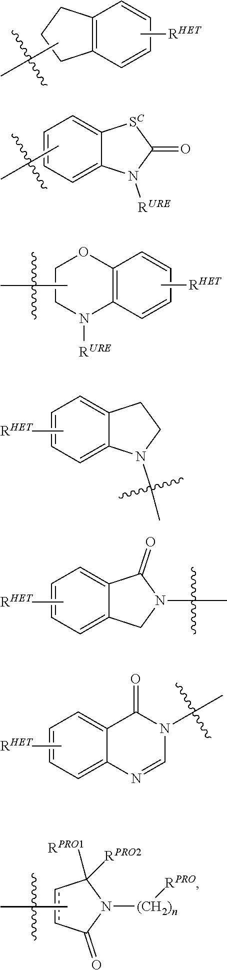

wherein:
S^{c} of ULM-g through ULM-i is CHR^{SS}, NR^{URE}, or O;
R^{HET} of ULM-g through ULM-i is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} of ULM-g through ULM-i is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁—C₆ alkyl) (preferably substituted with one or two hydroxyl groups or up to three halo groups);
R^{URE} of ULM-g through ULM-i is H, a C₁-C₆ alkyl (preferably H or C₁-C₃ alkyl) or a —C(O)(C₁-C₆ alkyl) each of which groups is optionally substituted with one or two hydroxyl groups or up to three halogen, preferably fluorine groups, or an optionally substituted phenyl group, an optionally substituted heteroaryl, or an optionally substituted heterocycle, preferably for example piperidine, morpholine, pyrrolidine, tetrahydrofuran);
R^{PRO} of ULM-g through ULM-i is H, optionally substituted C₁-C₆ alkyl or an optionally substituted aryl (phenyl or napthyl), heteroaryl or heterocyclic group selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, (each preferably substituted with a C₁-C₃ alkyl group, preferably methyl or a halo group, preferably F or Cl), benzofuran, indole, indolizine, azaindolizine;
R^{PRO1} and R^{PRO2} of ULM-g through ULM-i are each independently H, an optionally substituted C₁-C₃ alkyl group or together form a keto group; and
each n of ULM-g through ULM-i is independently 0, 1, 2, 3, 4, 5, or 6 (preferably 0 or 1), or an optionally substituted heterocycle, preferably tetrahydrofuran, tetrahydrothiene, piperidine, piperazine or morpholine (each of which groups when substituted, are preferably substituted with a methyl or halo (F, Br, Cl), each of which groups may be optionally attached to a PTM group (including a ULM′ group) via a linker group.

In certain preferred aspects,

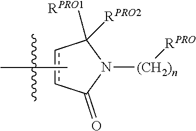

of ULM-g through ULM-i is a

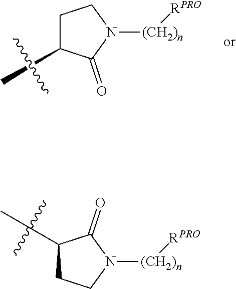

group,
where R^{PRO} and n of ULM-g through ULM-i are the same as above.

Preferred heteroaryl groups for R^{2′} of ULM-g through ULM-i include an optionally substituted quinoline (which may be attached to the pharmacophore or substituted on any carbon atom within the quinoline ring), an optionally substituted indole, an optionally substituted indolizine, an optionally substituted azaindolizine, an optionally substituted benzofuran, including an optionally substituted benzofuran, an optionally substituted isoxazole, an optionally substituted thiazole, an optionally substituted isothiazole, an optionally substituted thiophene, an optionally substituted pyridine (2-, 3, or 4-pyridine), an optionally substituted imidazole, an optionally substituted pyrrole, an optionally substituted diazole, an optionally substituted triazole, a tetrazole, an optionally substituted oximidazole, or a group according to the chemical structure:

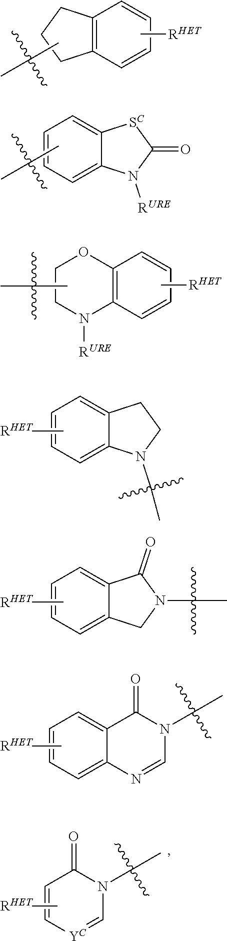

wherein:
S^{c} of ULM-g through ULM-i is CHR^{SS}, NR^{URE}, or O;
R^{HET} of ULM-g through ULM-i is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ of ULM-g through ULM-i is H or a C₁-C₆ alkyl group (preferably C₁-C₃ alkyl);
R^{SS} of ULM-g through ULM-i is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} of ULM-g through ULM-i is H, a C₁-C₆ alkyl (preferably H or C₁-C₃ alkyl) or a —C(O)(C₁-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted, and
Y^{C} of ULM-g through ULM-i is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl), each of which groups may be optionally connected to a PTM group (including a ULM′ group) via a linker group.

Preferred heterocycle groups for R² of ULM-g through ULM-i include tetrahydrofuran, tetrahydrothiene, tetrahydroquinoline, piperidine, piperazine, pyrrollidine, morpholine, oxane or thiane, each of which groups may be optionally substituted, or a group according to the chemical structure:

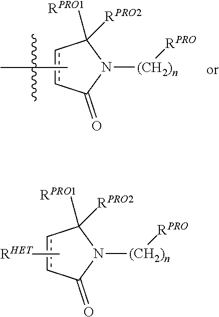

preferably, a

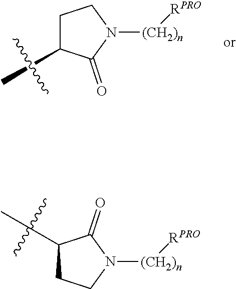

group,
wherein:
R^{PRO} of ULM-g through ULM-i is H, optionally substituted C₁-C₆ alkyl or an optionally substituted aryl, heteroaryl or heterocyclic group;
R^{PRO1} and R^{PRO2} of ULM-g through ULM-i are each independently H, an optionally substituted C₁-C₃ alkyl group or together form a keto group and
each n of ULM-g through ULM-i is independently 0, 1, 2, 3, 4, 5, or 6 (often 0 or 1), each of which groups may be optionally connected to a PTM group (including a ULM′ group) via a linker group.

Preferred R^{2′} substituents of ULM-g through ULM-i also include specifically (and without limitation to the specific compound disclosed) the R^{2′} substituents which are found in the identified compounds disclosed herein (which includes the specific compounds which are disclosed in the present specification, and the figures which are attached hereto). Each of these R^{2′} substituents may be used in conjunction with any number of R^{3′} substituents which are also disclosed herein.

R^{3′} of ULM-g through ULM-i is preferably an optionally substituted -T-Aryl, an optionally substituted-T-Heteroaryl, an optionally substituted -T-Heterocycle, an optionally substituted-NR¹-T-Aryl, an optionally substituted —NR¹-T-Heteroaryl or an optionally substituted-NR¹-T-Heterocycle, where R¹ is H or a C₁-C₃ alkyl group, preferably H or CH₃, T is an optionally substituted —(CH₂)ₙ— group, wherein each one of the methylene groups may be optionally substituted with one or two substituents, preferably selected from halogen, a C₁-C₃ alkyl group or the sidechain of an amino acid as otherwise described herein, preferably methyl, which may be optionally substituted; and n is 0 to 6, often 0, 1, 2, or 3 preferably 0 or 1. Alternatively, T may also be a —(CH₂O)ₙ— group, a —(OCH₂)ₙ— group, a —(CH₂CH₂O)ₙ— group, a —(OCH₂CH₂)ₙ— group, each of which groups is optionally substituted.

Preferred aryl groups for R^{3′} of ULM-g through ULM-i include optionally substituted phenyl or naphthyl groups, preferably phenyl groups, wherein the phenyl or naphthyl group is optionally connected to a PTM group (including a ULM′ group) via a linker group and/or optionally substituted with a halogen (preferably F or CO, an amine, monoalkyl- or dialkyl amine (preferably, dimethylamine), an amido group (preferably a —(CH₂)ₘ—NR₁C(O)R₂ group where m, R₁ and R₂ are the same as above), a halo (often F or CO, OH, CH₃, CF₃, OMe, OCF₃, NO₂, CN or a S(O)₂R_{S} group (R_{S} is a C₁-C₆ alkyl group, an optionally substituted aryl, heteroaryl or heterocycle group or a —(CH₂)ₘNR₁R₂ group), each of which may be substituted in ortho-, meta- and/or para-positions of the phenyl ring, preferably para-), or an Aryl (preferably phenyl), Heteroaryl or Heterocycle. Preferably said substituent phenyl group is an optionally substituted phenyl group (i.e., the substituent phenyl group itself is preferably substituted with at least one of F, Cl, OH, SH, COOH, CH₃, CF₃, OMe, OCF₃, NO₂, CN or a linker group to which is attached a PTM group (including a ULM′ group), wherein the substitution occurs in ortho-, meta- and/or para-positions of the phenyl ring, preferably para-), a naphthyl group, which may be optionally substituted including as described above, an optionally substituted heteroaryl (preferably an optionally substituted isoxazole including a methylsubstituted isoxazole, an optionally substituted oxazole including a methylsubstituted oxazole, an optionally substituted thiazole including a methyl substituted thiazole, an optionally substituted pyrrole including a methylsubstituted pyrrole, an optionally substituted imidazole including a methylimidazole, a benzylimidazole or methoxybenzylimidazole, an oximidazole or methyloximidazole, an optionally substituted diazole group, including a methyldiazole group, an optionally substituted triazole group, including a methylsubstituted triazole group, a pyridine group, including a halo-(preferably, F) or methylsubstitutedpyridine group or an oxapyridine group (where the pyridine group is linked to the phenyl group by an oxygen) or an optionally substituted heterocycle (tetrahydrofuran, tetrahydrothiophene, pyrrolidine, piperidine, morpholine, piperazine, tetrahydroquinoline, oxane or thiane. Each of the aryl, heteroaryl or heterocyclic groups may be optionally connected to a PTM group (including a ULM′ group) via a linker group.

Preferred Heteroaryl groups for R³ of ULM-g through ULM-i include an optionally substituted quinoline (which may be attached to the pharmacophore or substituted on any carbon atom within the quinoline ring), an optionally substituted indole (including dihydroindole), an optionally substituted indolizine, an optionally substituted azaindolizine (2, 3 or 4-azaindolizine) an optionally substituted benzimidazole, benzodiazole, benzoxofuran, an optionally substituted imidazole, an optionally substituted isoxazole, an optionally substituted oxazole (preferably methyl substituted), an optionally substituted diazole, an optionally substituted triazole, a tetrazole, an optionally substituted benzofuran, an optionally substituted thiophene, an optionally substituted thiazole (preferably methyl and/or thiol substituted), an optionally substituted isothiazole, an optionally substituted triazole (preferably a 1,2,3-triazole substituted with a methyl group, a triisopropylsilyl group, an optionally substituted —(CH₂)ₘ—O—C₁-C₆ alkyl group or an optionally substituted —(CH₂)ₘ—C(O)—O—C₁-C₆ alkyl group), an optionally substituted pyridine (2-, 3, or 4-pyridine) or a group according to the chemical structure:

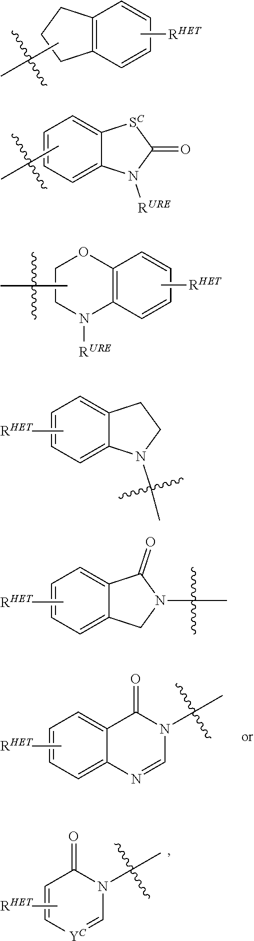

wherein:
S^{c} of ULM-g through ULM-i is CHR^{SS}, NR^{URE}, or O;
R^{HET} of ULM-g through ULM-i is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} of ULM-g through ULM-i is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} of ULM-g through ULM-i is H, a C₁-C₆ alkyl (preferably H or C₁-C₃ alkyl) or a —C(O)(C₁-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted, and
Y^{C} of ULM-g through ULM-i is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl). Each of said heteroaryl groups may be optionally connected to a PTM group (including a ULM′ group) via a linker group.

Preferred heterocycle groups for R³ of ULM-g through ULM-i include tetrahydroquinoline, piperidine, piperazine, pyrrollidine, morpholine, tetrahydrofuran, tetrahydrothiophene, oxane and thiane, each of which groups may be optionally substituted or a group according to the chemical structure:

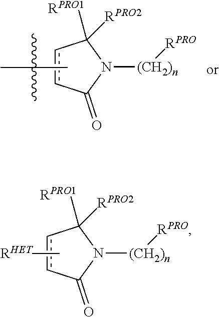

preferably, a

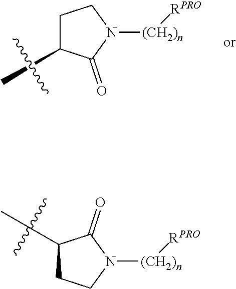

group,
wherein:
R^{PRO} of ULM-g through ULM-i is H, optionally substituted C₁-C₆ alkyl or an optionally substituted aryl (phenyl or napthyl), heteroaryl or heterocyclic group selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, (each preferably substituted with a C₁-C₃ alkyl group, preferably methyl or a halo group, preferably F or Cl), benzofuran, indole, indolizine, azaindolizine;
R^{PRO1} and R^{PRO2} of ULM-g through ULM-i are each independently H, an optionally substituted C₁-C₃ alkyl group or together form a keto group, and
each n of ULM-g through ULM-i is 0, 1, 2, 3, 4, 5, or 6 (preferably 0 or 1), wherein each of said Heteocycle groups may be optionally connected to a PTM group (including a ULM′ group) via a linker group.

Preferred R^{3′} substituents of ULM-g through ULM-i also include specifically (and without limitation to the specific compound disclosed) the R^{3′} substituents which are found in the identified compounds disclosed herein (which includes the specific compounds which are disclosed in the present specification, and the figures which are attached hereto). Each of these R^{3′} substituents may be used in conjunction with any number of R^{2′} substituents, which are also disclosed herein.

In certain alternative preferred embodiments, R^{2′} of ULM-g through ULM-i is an optionally substituted —NR₁—X^{R2′}-alkyl group, —NR₁—X^{R2′}-Aryl group; an optionally substituted —NR₁— X^{R2′}-HET, an optionally substituted —NR₁—X^{R2′}-Aryl-HET or an optionally substituted —NR₁— X^{R2′}-HET-Aryl,

wherein:
R₁ of ULM-g through ULM-i is H or a C₁-C₃ alkyl group (preferably H);
X^{R2′} of ULM-g through ULM-i is an optionally substituted —CH₂)ₙ—, —CH₂)ₙ—CH(Xᵥ)═CH(Xᵥ)— (cis or trans), —(CH₂)ₙ—CH≡CH—, —(CH₂CH₂O)ₙ— or a C₃-C₆ cycloalkyl group; and
Xᵥ of ULM-g through ULM-i is H, a halo or a C₁-C₃ alkyl group which is optionally substituted with one or two hydroxyl groups or up to three halogen groups;
Alkyl of ULM-g through ULM-i is an optionally substituted C1-C₁₀ alkyl (preferably a C₁-C₆ alkyl) group (in certain preferred embodiments, the alkyl group is end-capped with a halo group, often a Cl or Br);
Aryl of ULM-g through ULM-i is an optionally substituted phenyl or naphthyl group (preferably, a phenyl group); and
HET of ULM-g through ULM-i is an optionally substituted oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, benzofuran, indole, indolizine, azaindolizine, quinoline (when substituted, each preferably substituted with a C₁-C₃ alkyl group, preferably methyl or a halo group, preferably F or Cl) or a group according to the chemical structure:

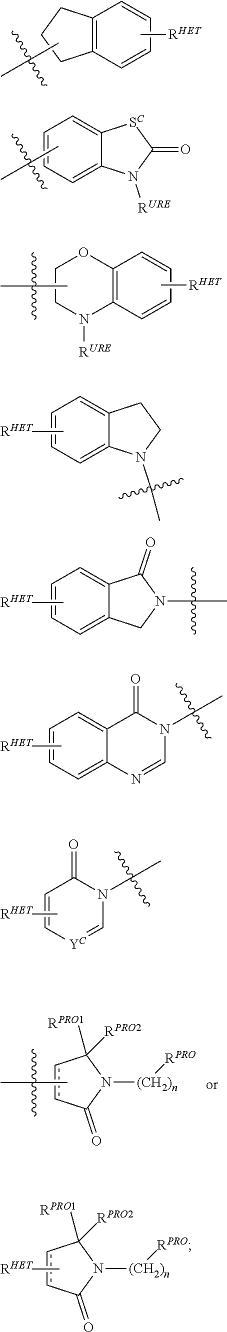

S^{c} of ULM-g through ULM-i is CHR^{SS}, NR^{URE}, or O;
R^{HET} of ULM-g through ULM-i is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} of ULM-g through ULM-i is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} of ULM-g through ULM-i is H, a C₁-C₆ alkyl (preferably H or C₁-C₃ alkyl) or a —C(O)(C₁-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted;
Y^{C} of ULM-g through ULM-i is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{PRO} of ULM-g through ULM-i is H, optionally substituted C₁-C₆ alkyl or an optionally substituted aryl (phenyl or napthyl), heteroaryl or heterocyclic group selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, (each preferably substituted with a C₁-C₃ alkyl group, preferably methyl or a halo group, preferably F or Cl), benzofuran, indole, indolizine, azaindolizine;
R^{PRO1} and R^{PRO2} of ULM-g through ULM-i are each independently H, an optionally substituted C₁-C₃ alkyl group or together form a keto group, and
each n of ULM-g through ULM-i is independently 0, 1, 2, 3, 4, 5, or 6 (preferably 0 or 1).

Each of said groups may be optionally connected to a PTM group (including a ULM′ group) via a linker group.

In certain alternative preferred embodiments of the present disclosure, R^{3′} of ULM-g through ULM-i is an optionally substituted —(CH₂)ₙ—(V)_{n′}—(CH₂)ₙ—(V)_{n′}—R^{s3′} group, an optionally substituted-(CH₂)ₙ—N(R_{1′})(C═O)_{m′} —(V)_{n′} —R^{S3′} group, an optionally substituted —X^{R3′}-alkyl group, an optionally substituted —X^{R3′}-Aryl group; an optionally substituted —X^{R3′}-HET group, an optionally substituted —X^{R3′}-Aryl-HET group or an optionally substituted —X^{R3′}-HET-Aryl group,

wherein:
R^{S3′} is an optionally substituted alkyl group (C₁-C₁₀, preferably C₁-C₆ alkyl), an optionally substituted Aryl group or a HET group;
R_{1′} is H or a C₁-C₃ alkyl group (preferably H);
V is O, S or NR₁;
X^{R3} is —(CH₂)ₙ—, —(CH₂CH₂O)ₙ—, —CH₂)ₙ—CH(Xᵥ)═CH(Xᵥ)— (cis or trans), —CH₂)ₙ—CH≡CH—, or a C₃-C₆ cycloalkyl group, all optionally substituted;
Xᵥ is H, a halo or a C₁-C₃ alkyl group which is optionally substituted with one or two hydroxyl groups or up to three halogen groups;
Alkyl is an optionally substituted C₁-C₁₀ alkyl (preferably a C₁-C₆ alkyl) group (in certain preferred embodiments, the alkyl group is end-capped with a halo group, often a Cl or Br);
Aryl is an optionally substituted phenyl or napthyl group (preferably, a phenyl group); and
HET is an optionally substituted oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, benzofuran, indole, indolizine, azaindolizine, quinoline (when substituted, each preferably substituted with a C₁-C₃ alkyl group, preferably methyl or a halo group, preferably F or Cl), or a group according to the chemical structure:

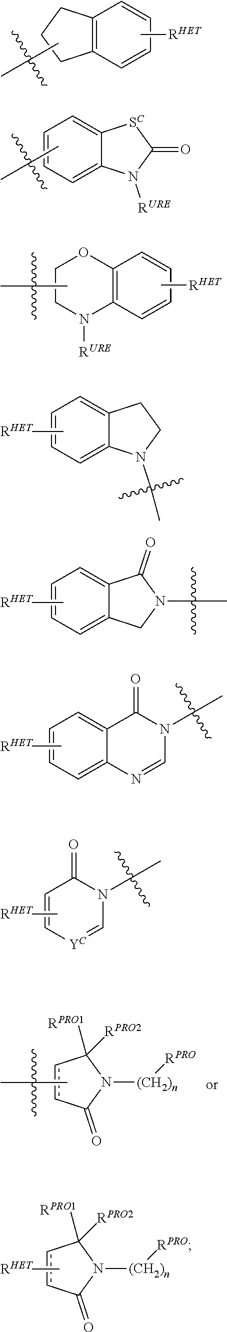

S^{c} of ULM-g through ULM-i is CHR^{SS}, NR^{URE}, or O;
R^{HET} of ULM-g through ULM-i is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} of ULM-g through ULM-i is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} of ULM-g through ULM-i is H, a C₁-C₆ alkyl (preferably H or C₁-C₃ alkyl) or a —C(O)(C₀-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted;
Y^{C} of ULM-g through ULM-i is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{PRO} of ULM-g through ULM-i is H, optionally substituted C₁-C₆ alkyl or an optionally substituted aryl (phenyl or napthyl), heteroaryl or heterocyclic group selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, (each preferably substituted with a C₁-C₃ alkyl group, preferably methyl or a halo group, preferably F or Cl), benzofuran, indole, indolizine, azaindolizine;
R^{PRO1} and R^{PRO2} of ULM-g through ULM-i are each independently H, an optionally substituted C₁-C₃ alkyl group or together form a keto group;
each n of ULM-g through ULM-i is independently 0, 1, 2, 3, 4, 5, or 6 (preferably 0 or 1);
each m′ of ULM-g through ULM-i is 0 or 1; and
each n′ of ULM-g through ULM-i is 0 or 1;
wherein each of said compounds, preferably on the alkyl, Aryl or Het groups, is optionally connected to a PTM group (including a ULM′ group) via a linker.

In alternative embodiments, R^{3′} of ULM-g through ULM-i is —(CH₂)ₙ-Aryl, —(CH₂CH₂O)ₙ-Aryl, —(CH₂)ₙ-HET or —(CH₂CH₂O)ₙ—HET,

wherein:
said Aryl of ULM-g through ULM-i is phenyl which is optionally substituted with one or two substitutents, wherein said substituent(s) is preferably selected from —(CH₂)ₙOH, C₁-C₆ alkyl which itself is further optionally substituted with CN, halo (up to three halo groups), OH, —(CH₂)ₙO(C₁-C₆)alkyl, amine, mono- or di-(C₁-C₆ alkyl) amine wherein the alkyl group on the amine is optionally substituted with 1 or 2 hydroxyl groups or up to three halo (preferably F, Cl) groups, or
said Aryl group of ULM-g through ULM-i is substituted with —(CH₂)ₙOH, —(CH₂)ₙ—O—(C₁-C₆)alkyl, —(CH₂)ₙ—O—(CH₂)ₙ—(C₁-C₆)alkyl, —(CH₂)ₙ—C(O)(C₀-C₆) alkyl, —(CH₂)ₙ—C(O)O(C₀-C₆)alkyl, —(CH₂)ₙ—OC(O)(C₀-C₆)alkyl, amine, mono- or di-(C₁-C₆ alkyl) amine wherein the alkyl group on the amine is optionally substituted with 1 or 2 hydroxyl groups or up to three halo (preferably F, Cl) groups, CN, NO₂, an optionally substituted —(CH₂)ₙ—(V)_{m′}—CH₂)ₙ—(V)_{m′}—(C₁-C₆)alkyl group, a —(V)_{m′}—(CH₂CH₂O)ₙ—R^{PEG} group where V is O, S or NR_{1′}, R_{1′} is H or a C₁-C₃ alkyl group (preferably H) and R^{PEG} is H or a C₁-C₆ alkyl group which is optionally substituted (including being optionally substituted with a carboxyl group), or
said Aryl group of ULM-g through ULM-i is optionally substituted with a heterocycle, including a heteroaryl, selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, benzofuran, indole, indolizine, azaindolizine, (when substituted each preferably substituted with a C₁-C₃ alkyl group, preferably methyl or a halo group, preferably F or Cl), or a group according to the chemical structure:

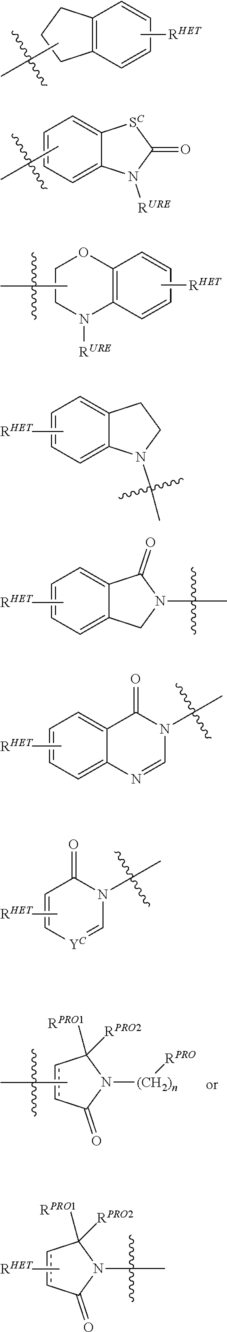

S^{c} of ULM-g through ULM-i is CHR^{SS}, NR^{URE}, or O;
R^{HET} of ULM-g through ULM-i is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} of ULM-g through ULM-i is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} of ULM-g through ULM-i is H, a C₁-C₆ alkyl (preferably H or C₁-C₃ alkyl) or a —C(O)(C₀-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted;
Y^{C} of ULM-g through ULM-i is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{PRO} of ULM-g through ULM-i is H, optionally substituted C₁-C₆ alkyl or an optionally substituted aryl (phenyl or napthyl), heteroaryl or heterocyclic group selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, (each preferably substituted with a C₁-C₃ alkyl group, preferably methyl or a halo group, preferably F or Cl), benzofuran, indole, indolizine, azaindolizine;
R^{PRO1} and R^{PRO2} of ULM-g through ULM-i are each independently H, an optionally substituted C₁-C₃ alkyl group or together form a keto group;
HET of ULM-g through ULM-i is preferably oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, (each preferably substituted with a C₁-C₃ alkyl group, preferably methyl or a halo group, preferably F or Cl), benzofuran, indole, indolizine, azaindolizine, or a group according to the chemical structure:

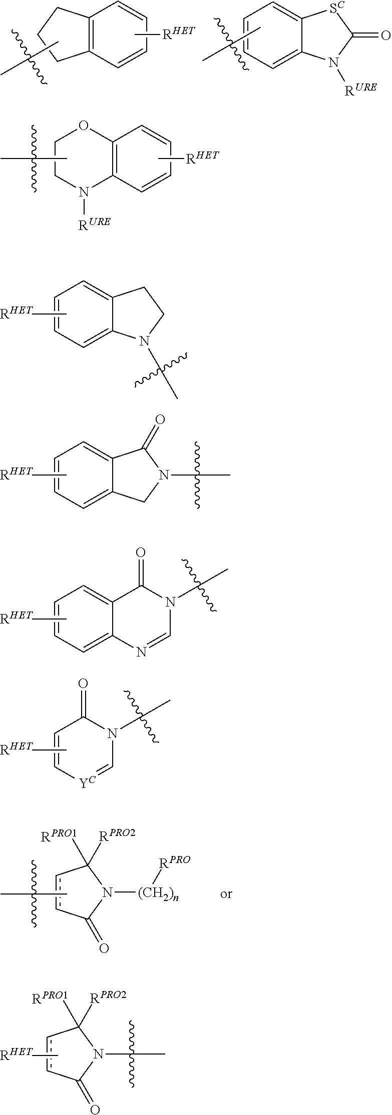

S^{c} of ULM-g through ULM-i is CHR^{SS}, NR^{URE}, or O;
R^{HET} of ULM-g through ULM-i is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} of ULM-g through ULM-i is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} of ULM-g through ULM-i is H, a C₁-C₆ alkyl (preferably H or C₁-C₃ alkyl) or a —C(O)(C₀-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted;
Y^{C} of ULM-g through ULM-i is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{PRO} of ULM-g through ULM-i is H, optionally substituted C₁-C₆ alkyl or an optionally substituted aryl, heteroaryl or heterocyclic group;
R^{PRO1} and R^{PRO2} of ULM-g through ULM-i are each independently H, an optionally substituted C₁-C₃ alkyl group or together form a keto group;
each m′ of ULM-g through ULM-i is independently 0 or 1; and
each n of ULM-g through ULM-i is independently 0, 1, 2, 3, 4, 5, or 6 (preferably 0 or 1),
wherein each of said compounds, preferably on said Aryl or HET groups, is optionally connected to a PTM group (including a ULM′ group) via a linker group.

In still additional embodiments, preferred compounds include those according to the chemical structure:

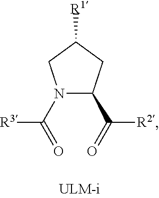

wherein:
R^{1′} of ULM-i is OH or a group which is metabolized in a patient or subject to OH;
R^{2′} of ULM-i is a —NH—CH₂-Aryl-HET (preferably, a phenyl linked directly to a methyl substituted thiazole);
R³ of ULM-i is a —CHR^{CR3′}—NH—C(O)—R^{3P1} group or a —CHR^{CR3′}—R^{3P2} group;
R^{CR3′} of ULM-i is a C₁-C₄ alkyl group, preferably methyl, isopropyl or tert-butyl;
R^{3P1} of ULM-i is C₁-C₃ alkyl (preferably methyl), an optionally substituted oxetane group (preferably methyl substituted, a —(CH₂)ₙOCH₃ group where n is 1 or 2 (preferably 2), or a

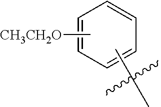

group (the ethyl ether group is preferably meta-substituted on the phenyl moiety), a morpholino group (linked to the carbonyl at the 2- or 3-position;
R^{3P2} of ULM-i is a

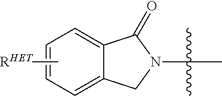

group;
Aryl of ULM-i is phenyl;
HET of ULM-i is an optionally substituted thiazole or isothiazole; and
R^{HET} of ULM-i is H or a halo group (preferably H);
or a pharmaceutically acceptable salt, stereoisomer, solvate or polymorph thereof, wherein each of said compounds is optionally connected to a PTM group (including a ULM′ group) via a linker group.

In certain aspects, bifunctional compounds comprising a ubiquitin E3 ligase binding moiety (ULM), wherein ULM is a group according to the chemical structure:

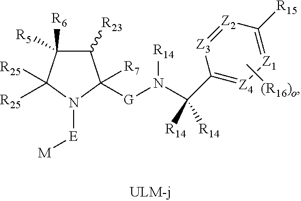

wherein:
each R₅ and R₆ of ULM-j is independently OH, SH, or optionally substituted alkyl or R₅, R₆, and the carbon atom to which they are attached form a carbonyl;
R₇ of ULM-j is H or optionally substituted alkyl;
E of ULM-j is a bond, C═O, or C═S;
G of ULM-j is a bond, optionally substituted alkyl, —COOH or C=J;

### J of ULM-j is O or N—R₈;

R₈ of ULM-j is H, CN, optionally substituted alkyl or optionally substituted alkoxy;
M of ULM-j is optionally substituted aryl, optionally substituted heteroaryl, optionally substituted heterocyclic or

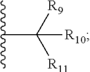

each R₉ and R₁₀ of ULM-j is independently H; optionally substituted alkyl, optionally substituted cycloalkyl, optionally substituted hydroxyalkyl, optionally substituted thioalkyl, a disulphide linked ULM, optionally substituted heteroaryl, or haloalkyl; or R₉, R₁₀, and the carbon atom to which they are attached form an optionally substituted cycloalkyl;
R₁₁ of ULM-j is optionally substituted heterocyclic, optionally substituted alkoxy, optionally substituted heteroaryl, optionally substituted aryl, or

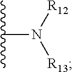

R₁₂ of ULM-j is H or optionally substituted alkyl;
R₁₃ of ULM-j is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl; optionally substituted (oxoalkyl)carbamate,
each R₁₄ of ULM-j is independently H, haloalkyl, optionally substituted cycloalkyl, optionally substituted alkyl or optionally substituted heterocycloalkyl;
R₁₅ of ULM-j is H, optionally substituted heteroaryl, haloalkyl, optionally substituted aryl, optionally substituted alkoxy, or optionally substituted heterocyclyl;
each R₁₆ of ULM-j is independently halo, optionally substituted alkyl, optionally substituted haloalkyl, CN, or optionally substituted haloalkoxy;
each R₂₅ of ULM-j is independently H or optionally substituted alkyl; or both R₂₅ groups can be taken together to form an oxo or optionally substituted cycloalkyl group;

### R₂₃ of ULM-j is H or OH;

Z₁, Z₂, Z₃, and Z₄ of ULM-j are independently C or N; and
o of ULM-j is 0, 1, 2, 3, or 4, or a pharmaceutically acceptable salt, stereoisomer, solvate or polymorph thereof.

In certain embodiments, wherein G of ULM-j is C=J, J is O, R₇ is H, each R₁₄ is H, and o is 0.

In certain embodiments, wherein G of ULM-j is C=J, J is O, R₇ is H, each R₁₄ is H, R₁₅ is optionally substituted heteroaryl, and o is 0. In other instances, E is C═O and M is

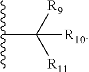

In certain embodiments, wherein E of ULM-j is C═O, R₁₁ is optionally substituted heterocyclic or

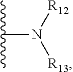

### and M is

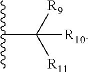

In certain embodiments, wherein E of ULM-j is C═O, M is

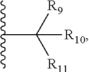

### and R₁₁ is

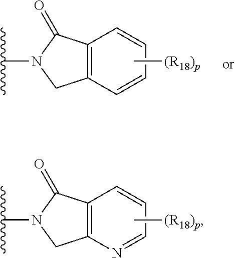

each R₁₈ is independently halo, optionally substituted alkoxy, cyano, optionally substituted alkyl, haloalkyl, or haloalkoxy; and p is 0, 1, 2, 3, or 4.

In certain embodiments, ULM and where present, ULM′, are each independently a group according to the chemical structure:

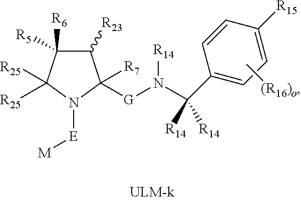

wherein:
G of ULM-k is C=J, J is O;
R₇ of ULM-k is H;
each R₁₄ of ULM-k is H;
o of ULM-k is 0;
R₁₅ of ULM-k is

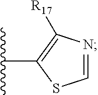

and
R₁₇ of ULM-k is H, halo, optionally substituted cycloalkyl, optionally substituted alkyl, optionally substituted alkenyl, and haloalkyl.

In other instances, R₁₇ of ULM-k is alkyl (e.g., methyl) or cycloalkyl (e.g., cyclopropyl).

In other embodiments, ULM and where present, ULM′, are each independently a group according to the chemical structure:

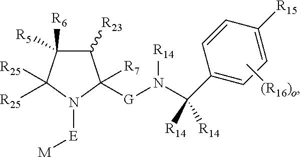

wherein:

G of ULM-k is C=J, J is O;

R₇ of ULM-k is H;

each R₁₄ of ULM-k is H;

o of ULM-k is 0; and

R₁₅ of ULM-k is selected from the group consisting of:

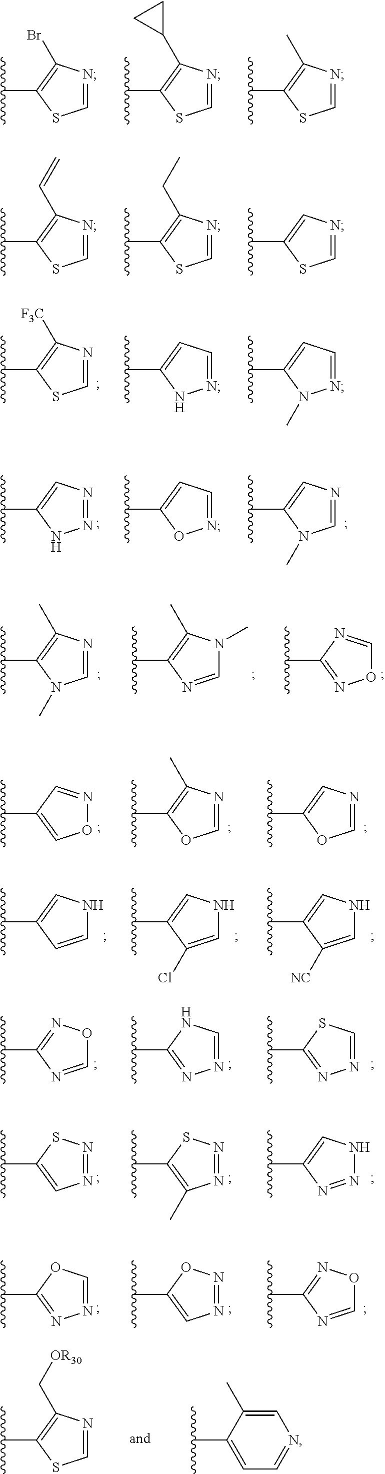

wherein R₃₀ of ULM-k is H or an optionally substituted alkyl.

In other embodiments, ULM and where present, ULM′, are each independently a group according to the chemical structure:

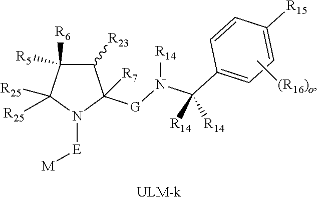

wherein:

E of ULM-k is C═O;

M of ULM-k is

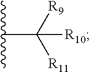

and

R₁₁ of ULM-k is selected from the group consisting of:

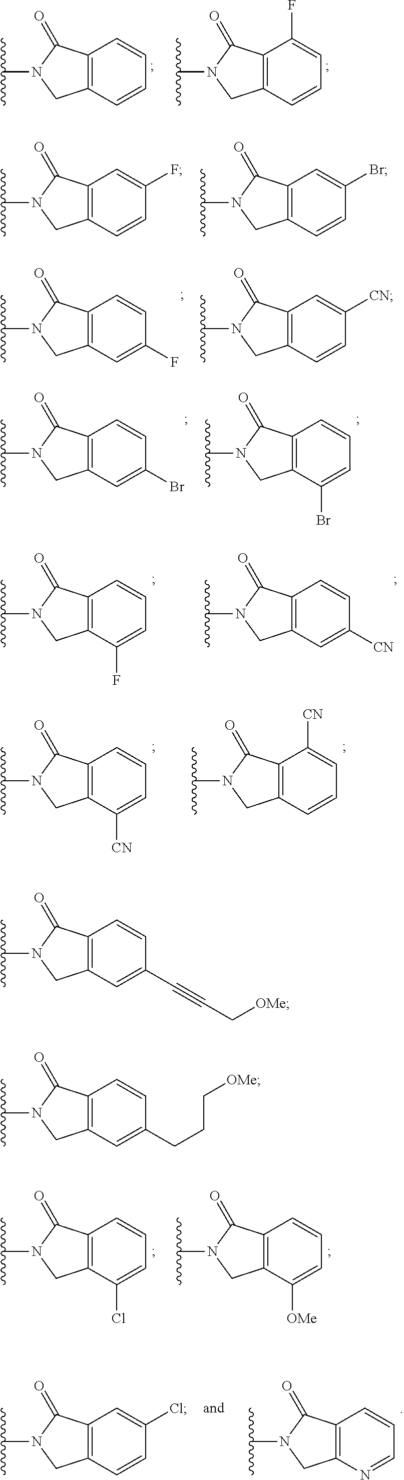

In still other embodiments, a compound of the chemical structure,

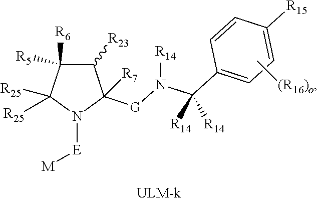

wherein E of ULM-k is C═O;

### R₁₁ of ULM-k is

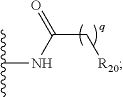

and

### M of ULM-k is

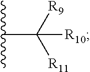

### q of ULM-k is 1 or 2;

R₂₀ of ULM-k is H, optionally substituted alkyl, optionally substituted cycloalkyl, optionally substituted aryl, or

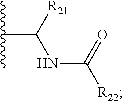

R₂₁ of ULM-k is H or optionally substituted alkyl; and
R₂₂ of ULM-k is H, optionally substituted alkyl, optionally substituted alkoxy, or haloalkyl.

In any embodiment described herein, R₁₁ of ULM-j or ULM-k is selected from the group consisting of:

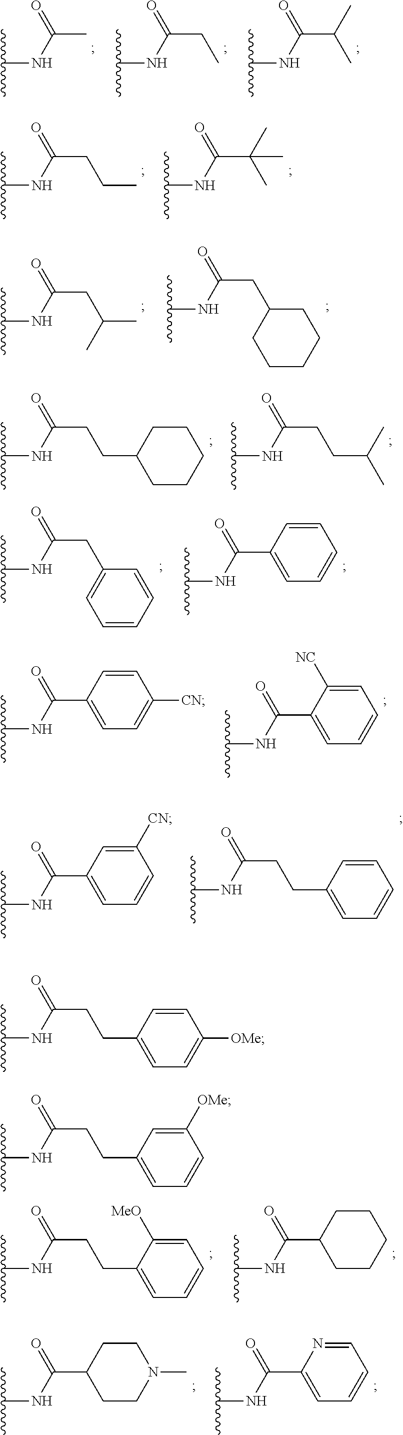

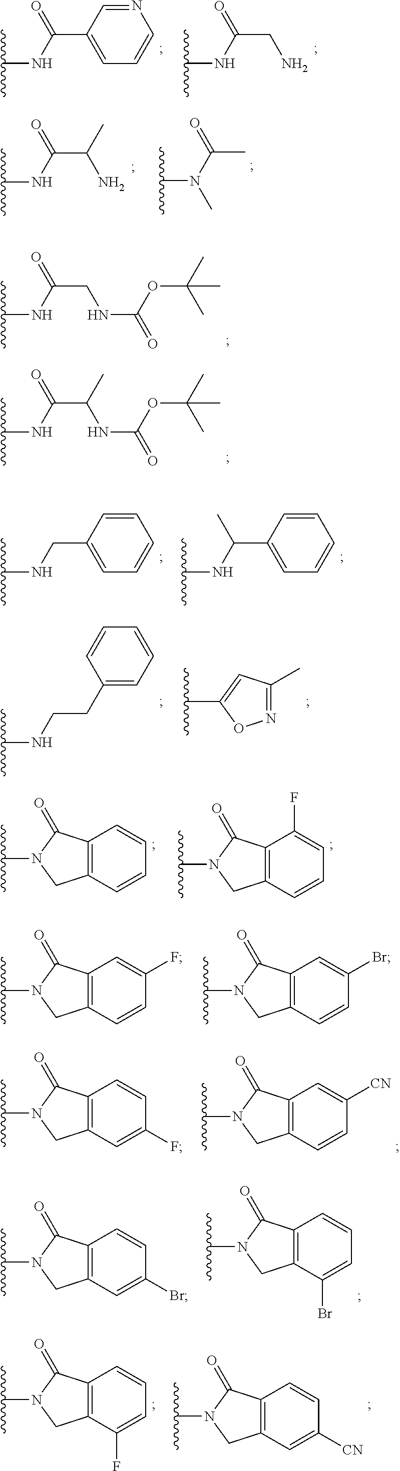

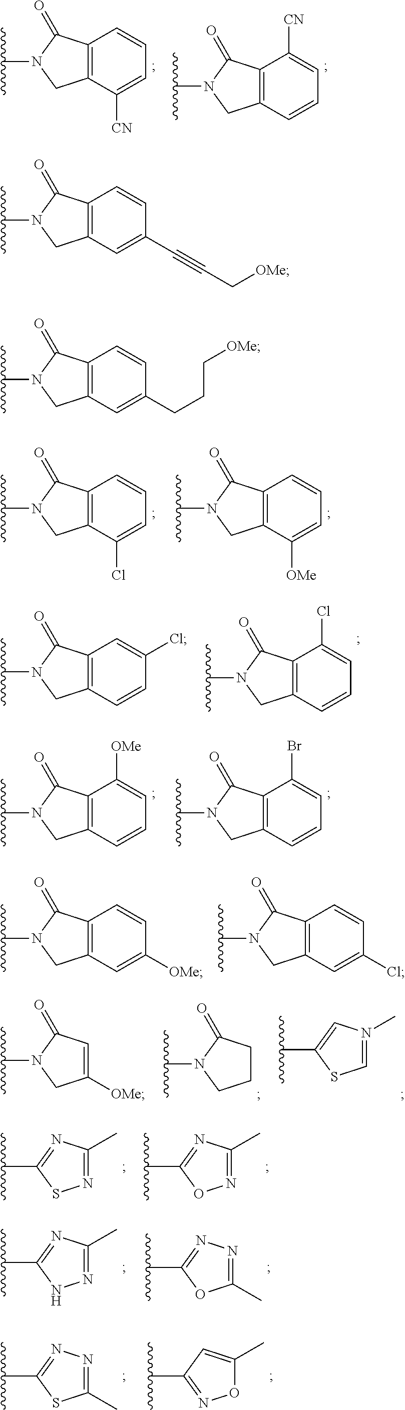

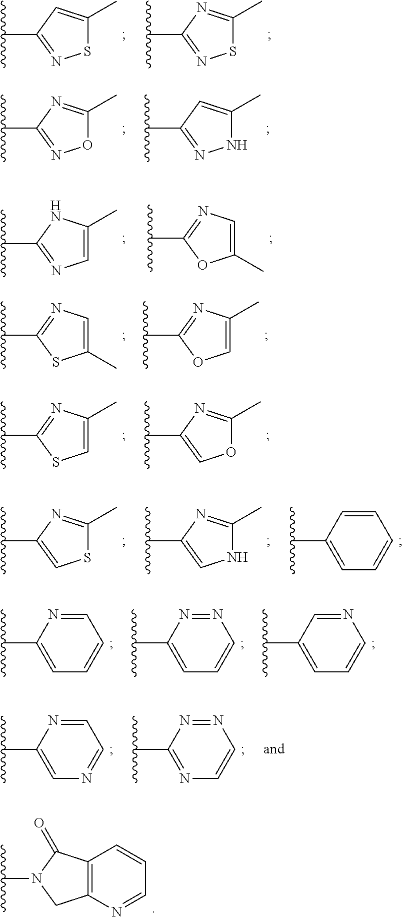

In certain embodiments, R₁₁ of ULM-j or ULM-k is selected from the group consisting of:

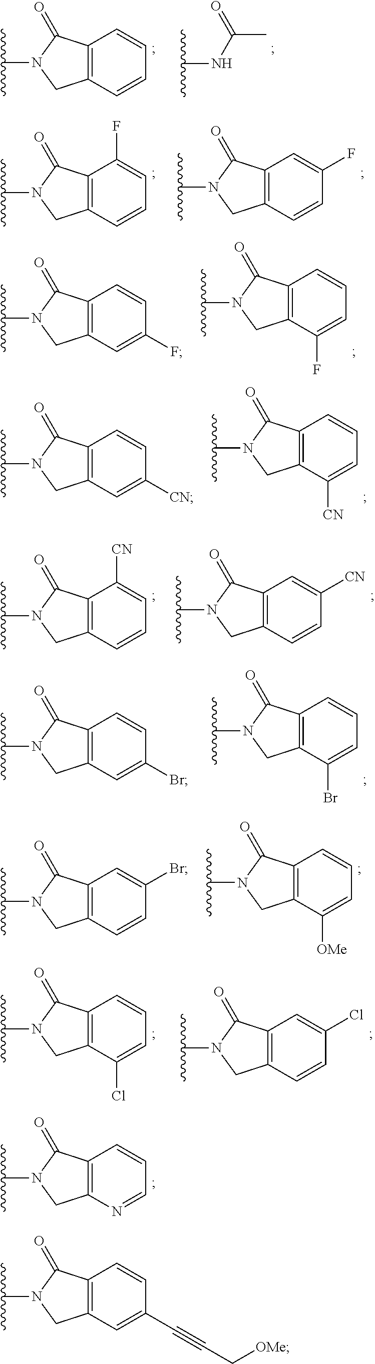

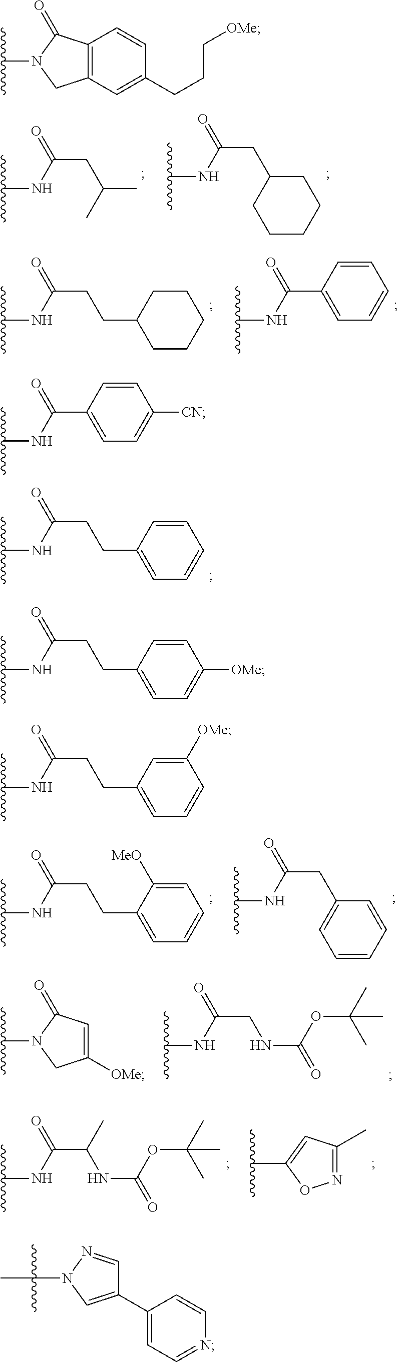

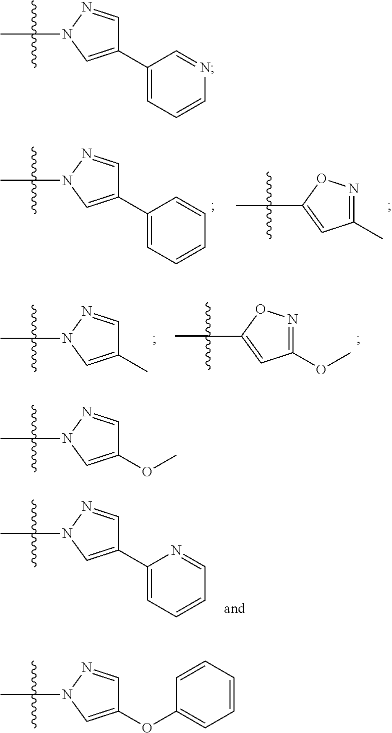

In certain embodiments, ULM (or when present ULM′) is a group according to the chemical structure:

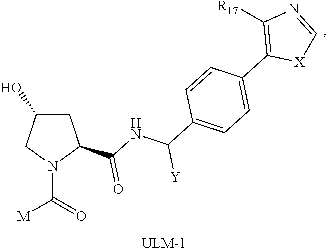

wherein:
X of ULM-1 is O or S;
Y of ULM-1 is H, methyl or ethyl;
R₁₇ of ULM-1 is H, methyl, ethyl, hydoxymethyl or cyclopropyl;
M of ULM-1 is optionally substituted aryl, optionally substituted heteroaryl, or

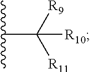

R₉ of ULM-1 is H;
R₁₀ of ULM-1 is H, optionally substituted alkyl, optionally substituted haloalkyl, optionally substituted heteroaryl, optionally substituted aryl, optionally substituted hydroxyalkyl, optionally substituted thioalkyl or cycloalkyl;
R11 of ULM-1 is optionally substituted heteroaromatic, optionally substituted heterocyclic, optionally substituted aryl or

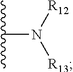

R₁₂ of ULM-1 is H or optionally substituted alkyl; and
R₁₃ of ULM-1 is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl; optionally substituted (oxoalkyl)carbamate.

In some embodiments, ULM and where present, ULM′, are each independently a group according to the chemical structure:

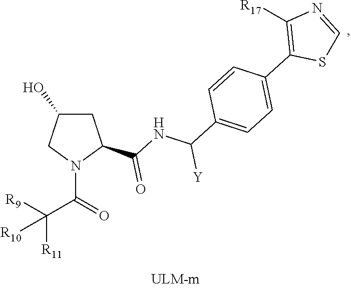

wherein:
Y of ULM-m is H, methyol or ethyl
R₉ of ULM-m is H;
R₁₀ is isopropyl, tert-butyl, sec-butyl, cyclopentyl, or cyclohexyl;
R₁₁ of ULM-m is optionally substituted amide, optionally substituted isoindolinone, optionally substituted isooxazole, optionally substituted heterocycles.

In other preferred embodiments of the disclosure, ULM and where present, ULM′, are each independently a group according to the chemical structure:

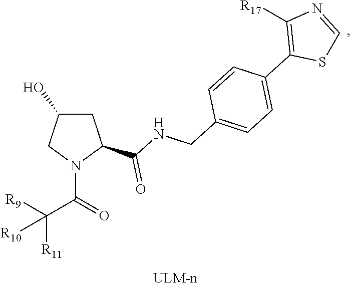

wherein:
R₁₇ of ULM-n is methyl, ethyl, or cyclopropyl; and
R₉, R₁₀, and R₁₁ of ULM-n are as defined above. In other instances, R₉ is H; and
R₁₀ of ULM-n is H, alkyl, or cycloalkyl (preferably, isopropyl, tert-butyl, sec-butyl, cyclopentyl, or cyclohexyl).

In any of the aspects or embodiments described herein, the ULM (or when present, ULM′) as described herein may be a pharmaceutically acceptable salt, enantiomer, diastereomer, solvate or polymorph thereof. In addition, in any of the aspects or embodiments described herein, the ULM (or when present, ULM′) as described herein may be coupled to a PTM directly via a bond or by a chemical linker.

In certain aspects of the disclosure, the ULM moiety is selected from the group consisting of:

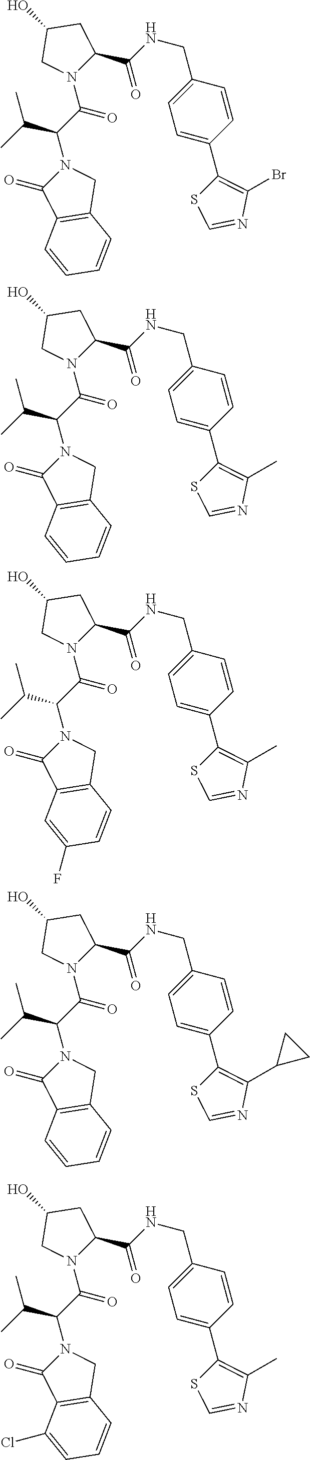

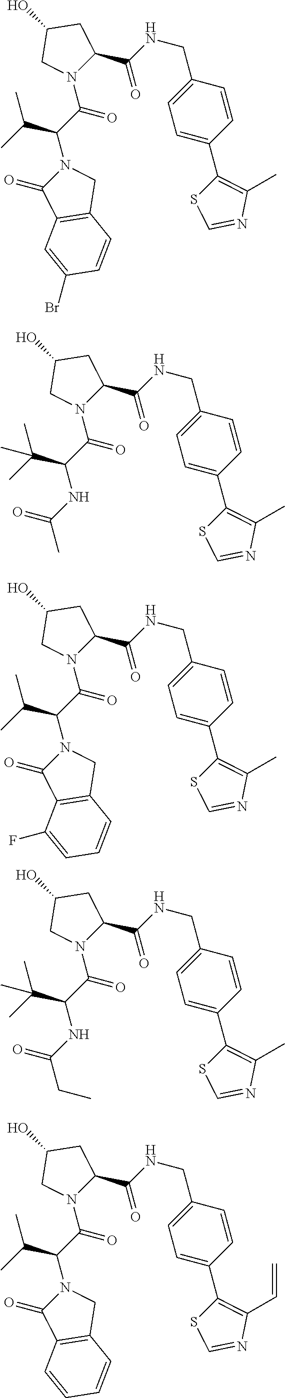

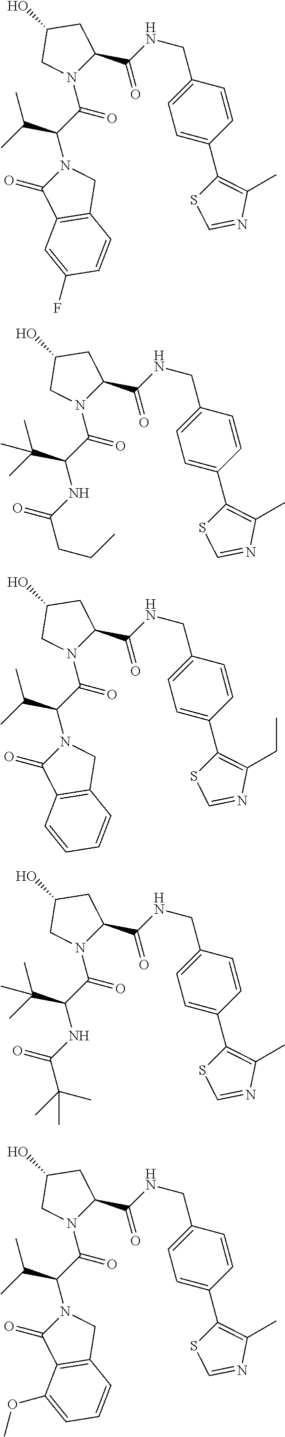

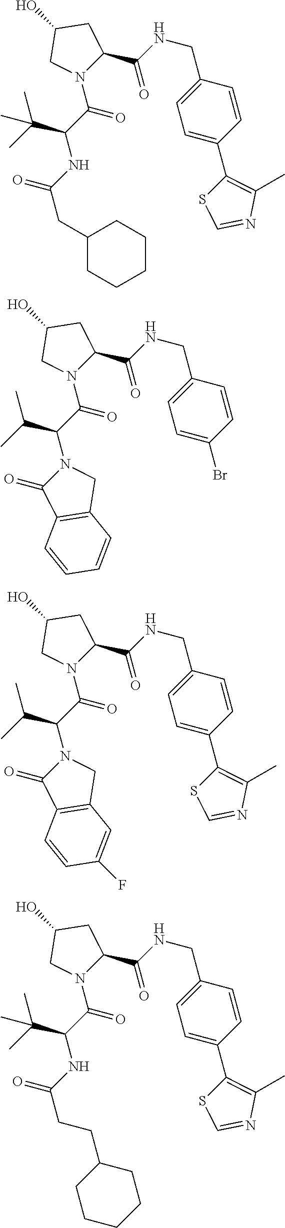

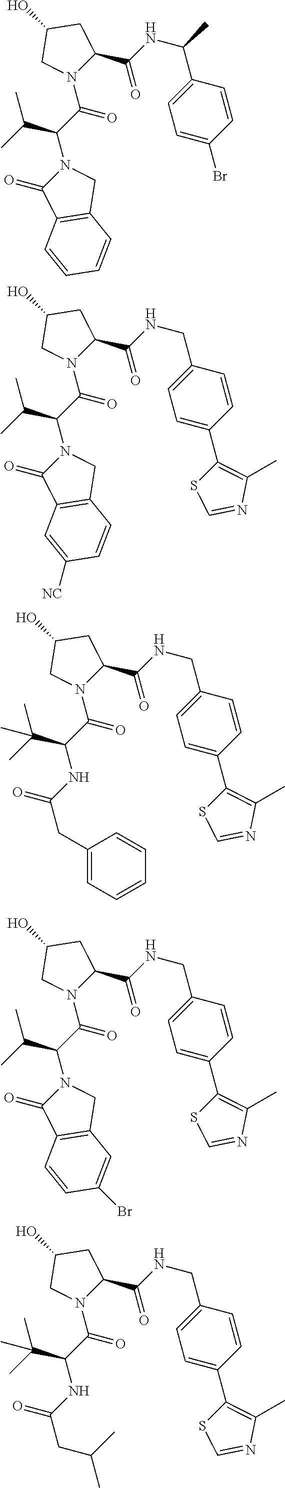

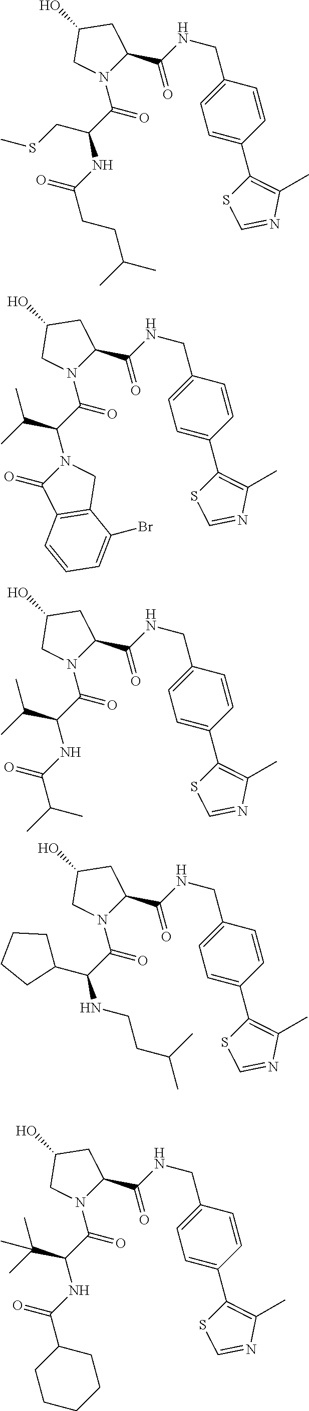

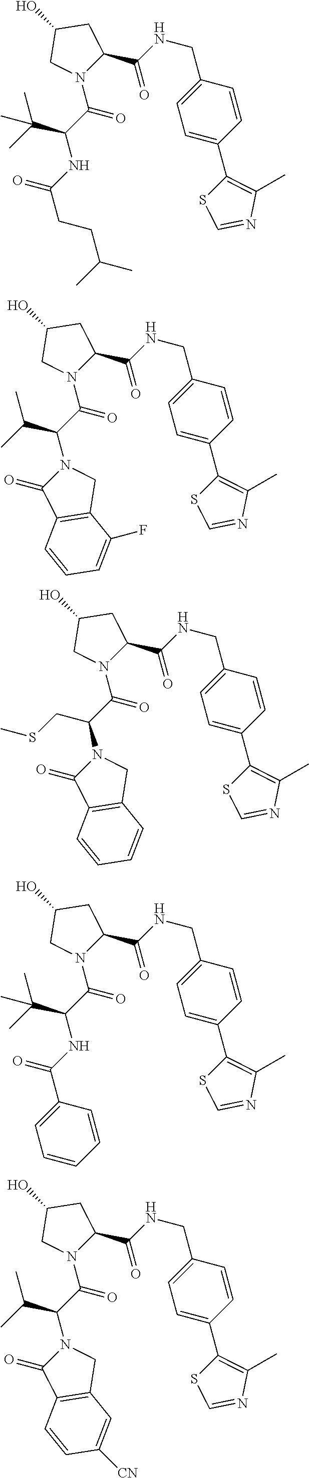

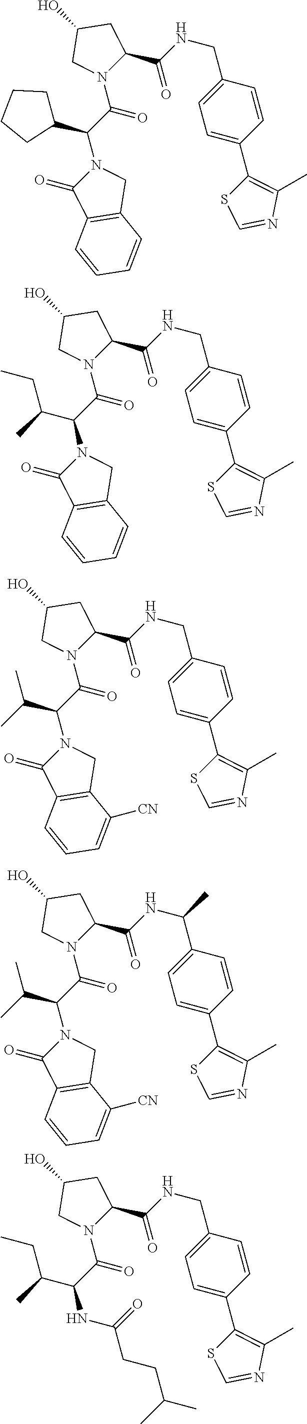

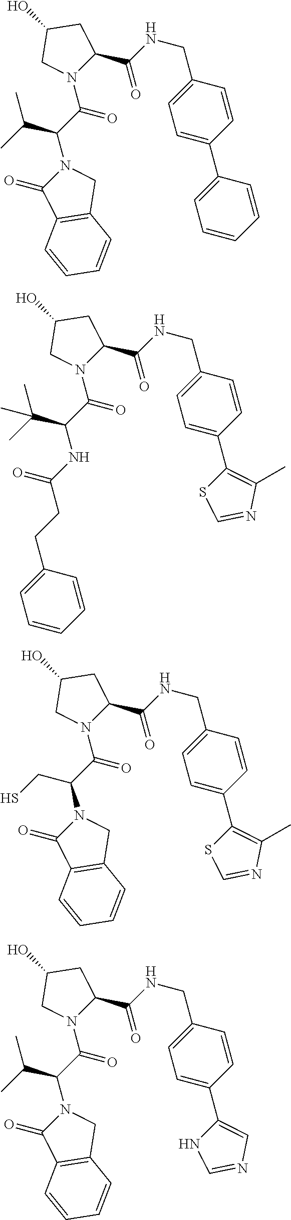

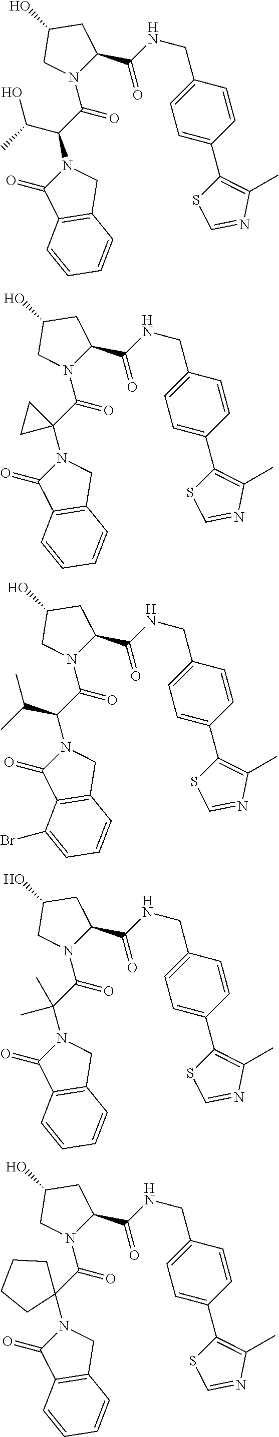

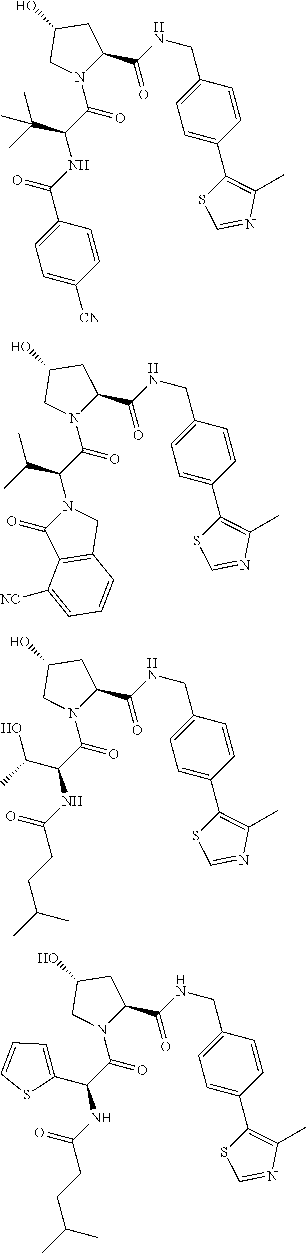

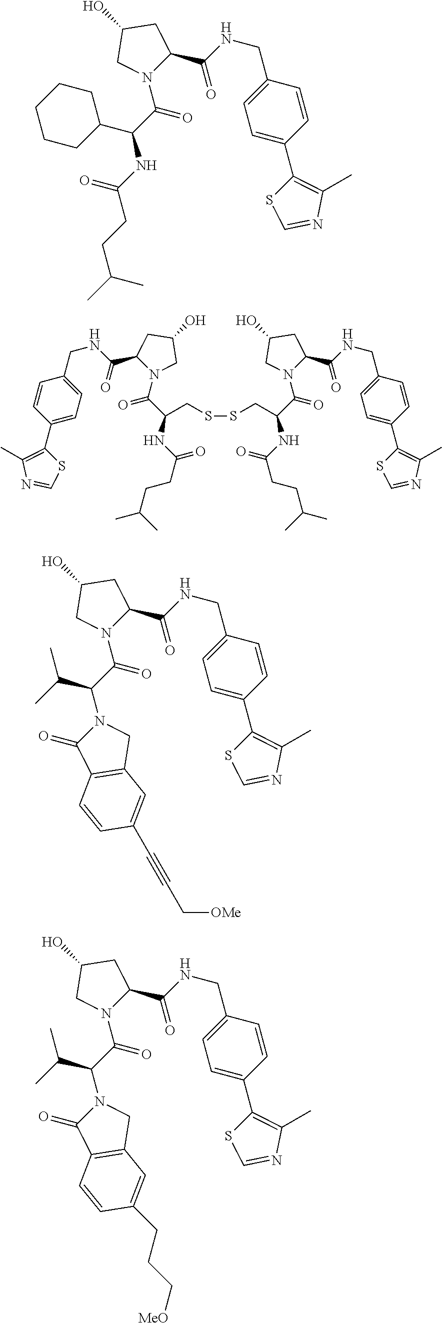

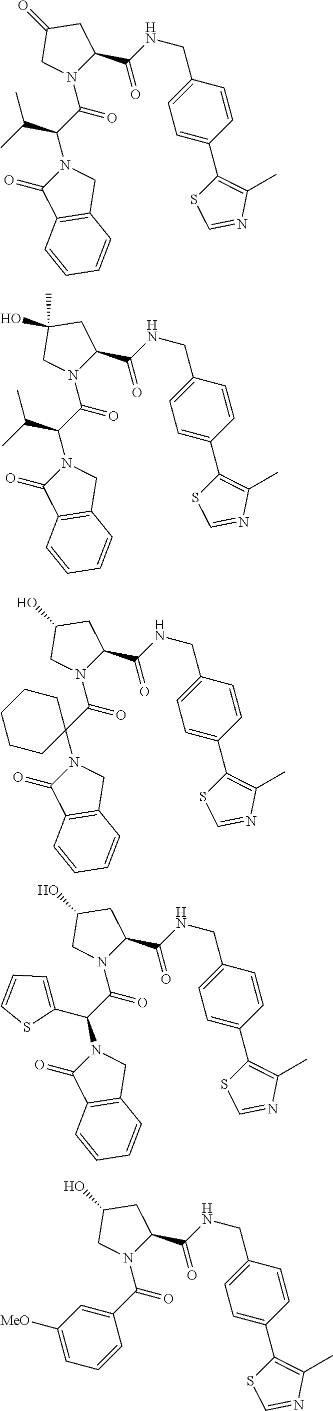

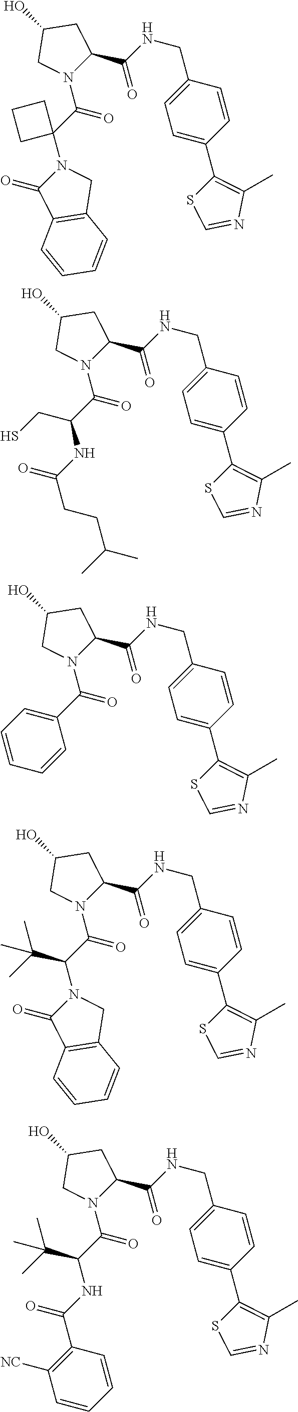

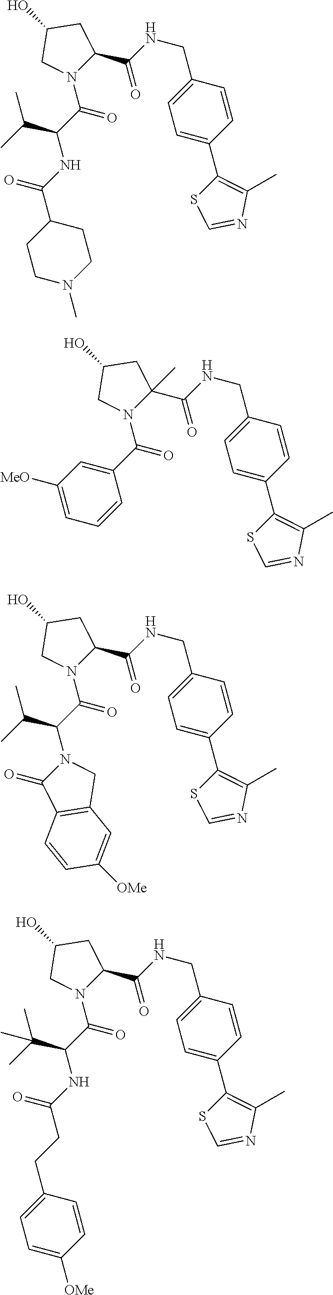

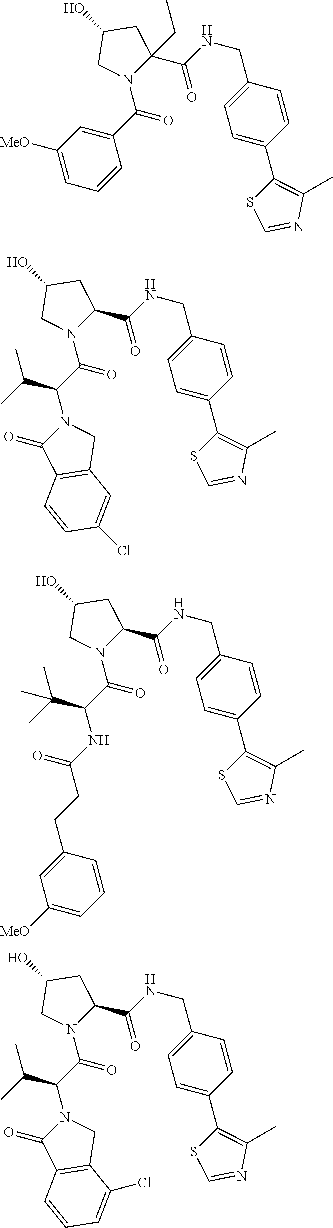

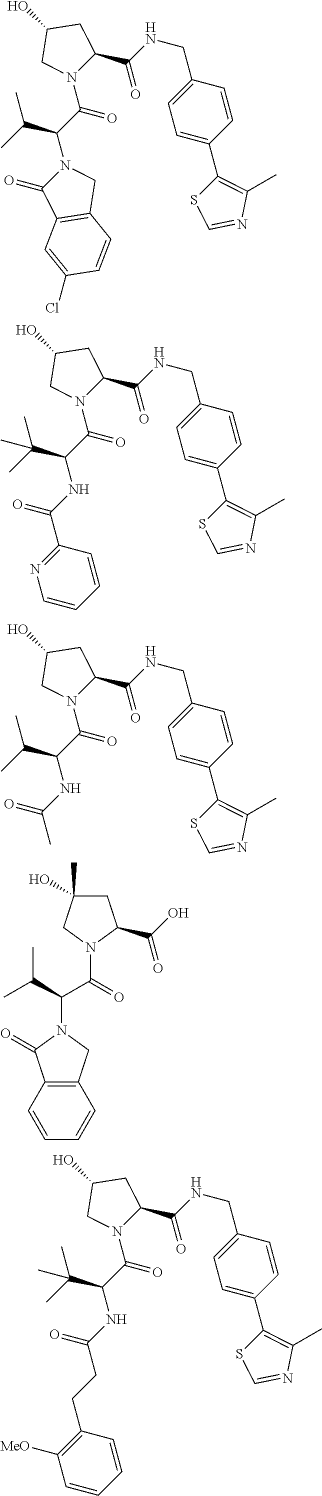

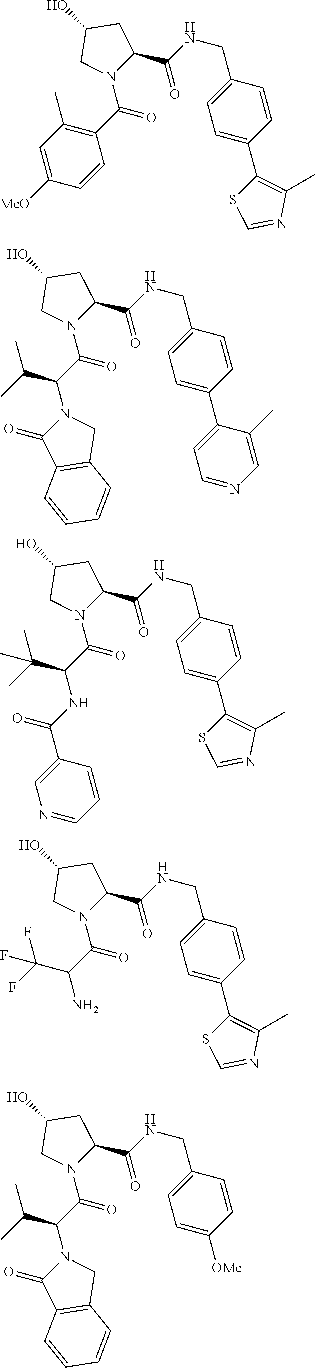

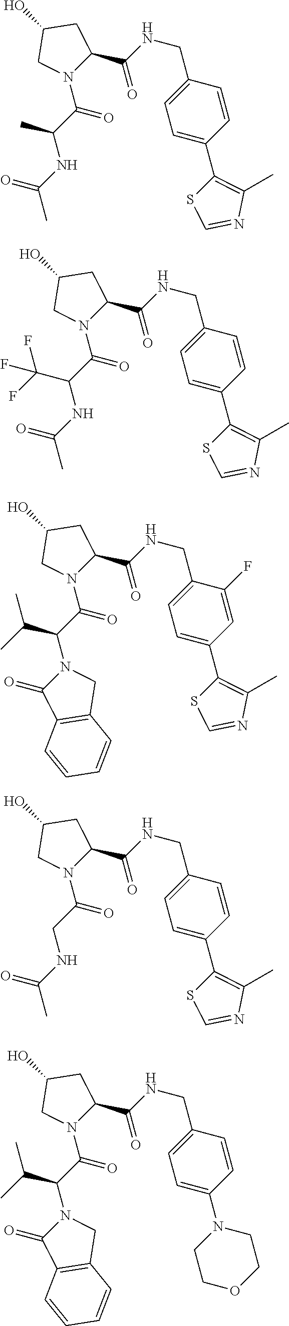

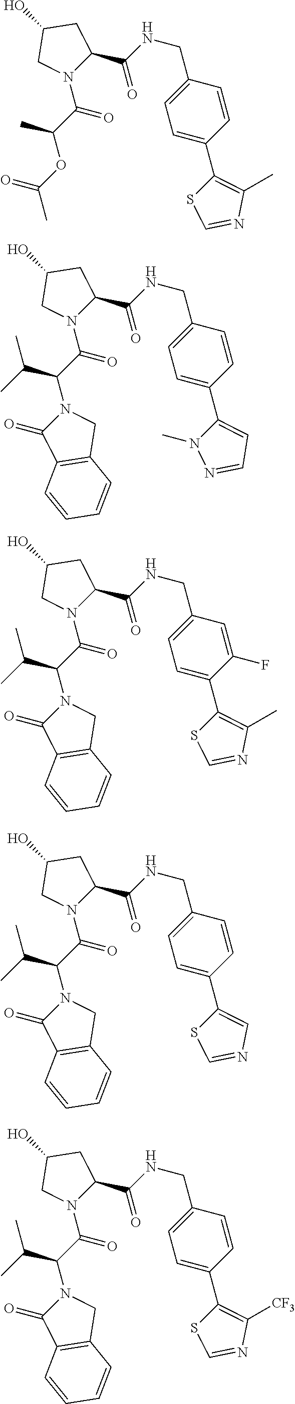

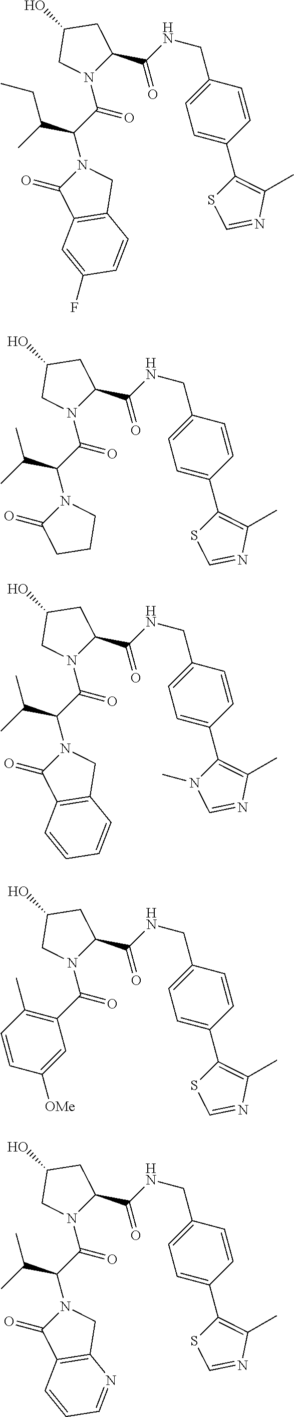

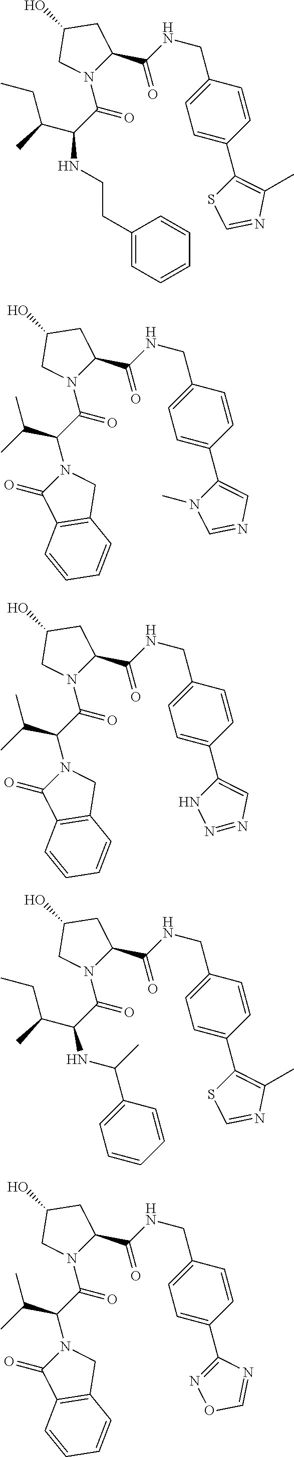

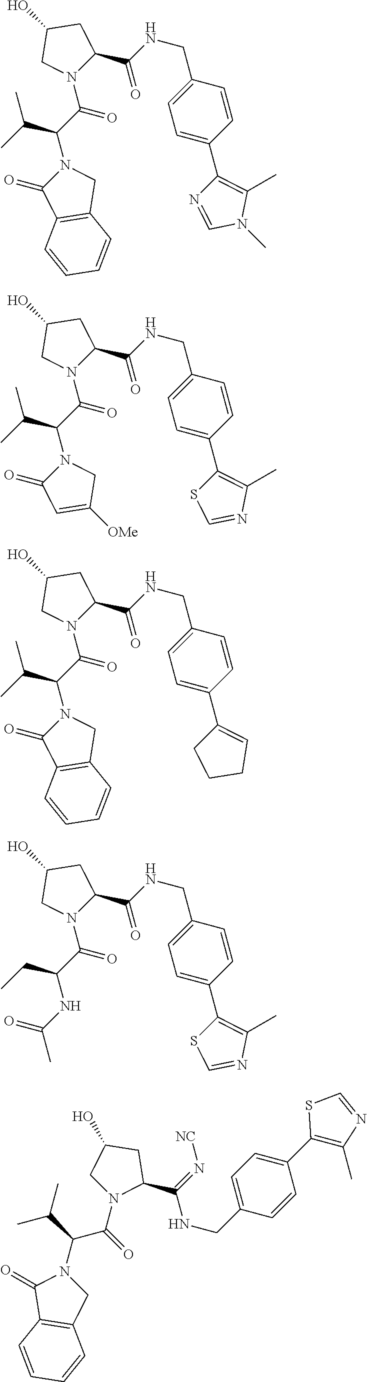

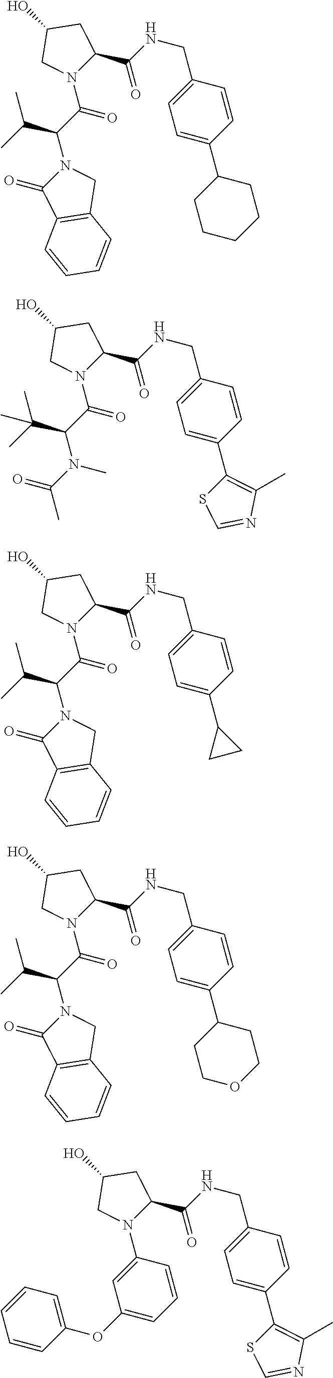

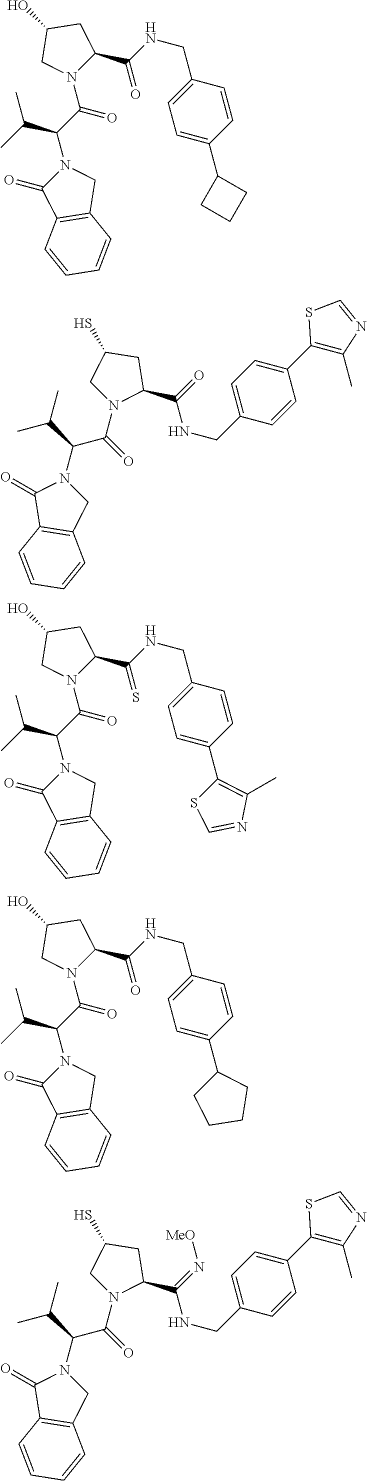

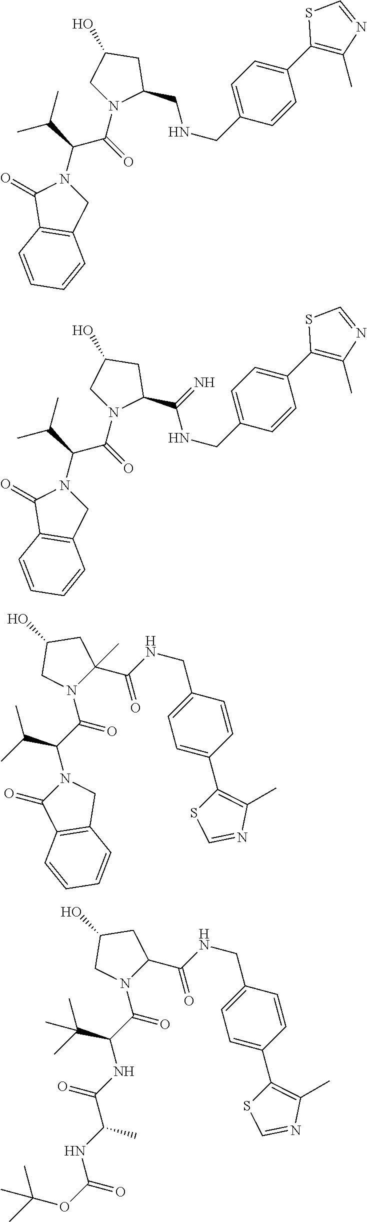

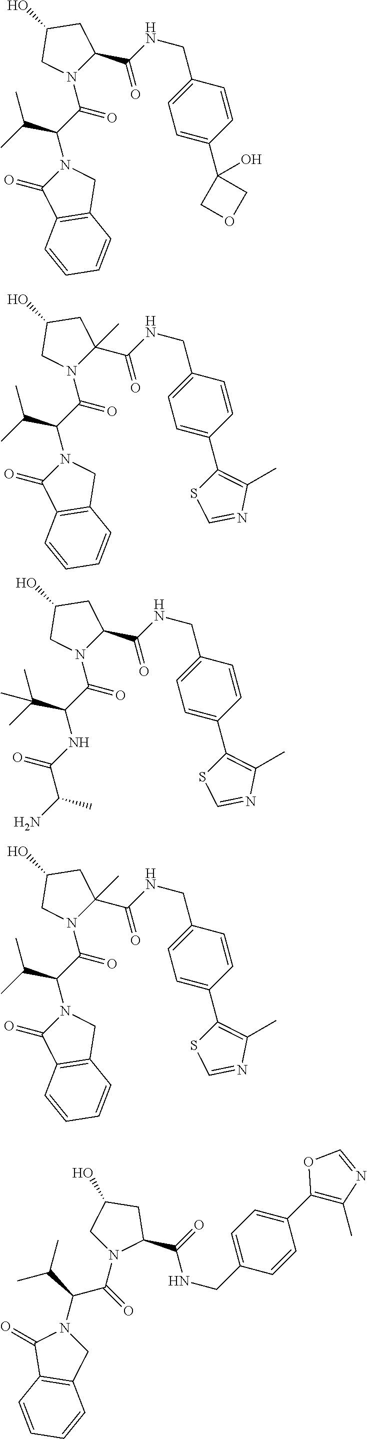

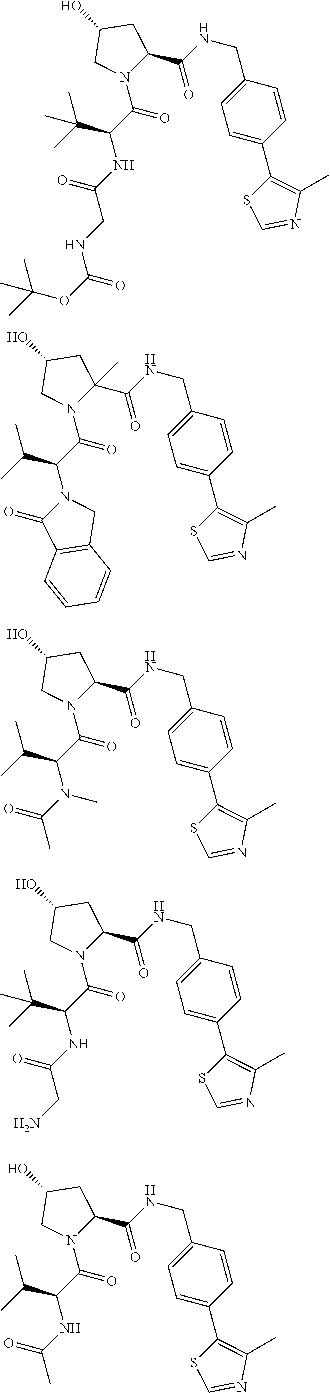

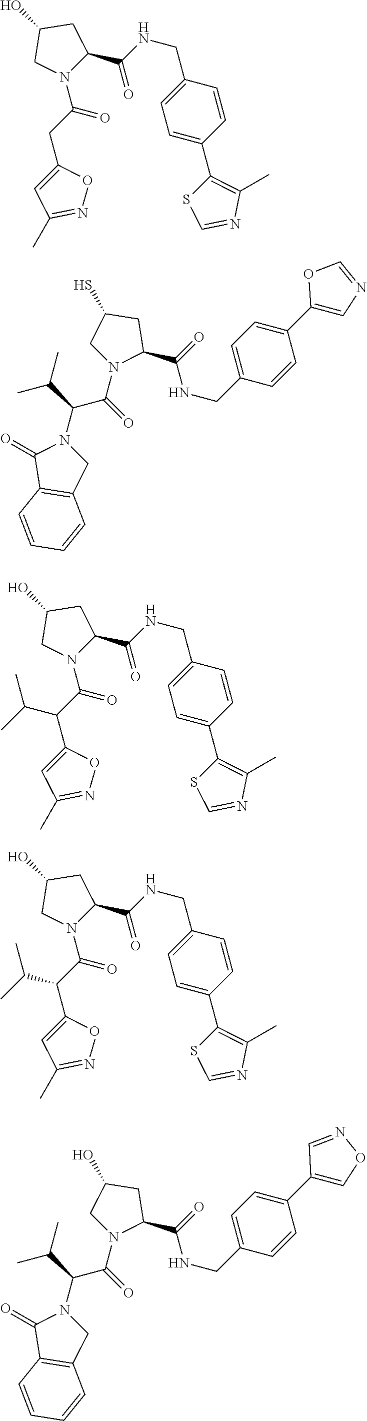

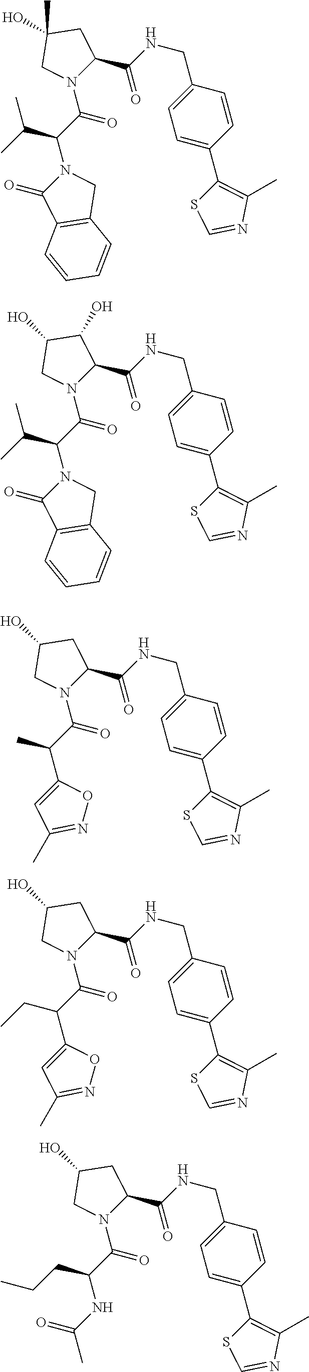

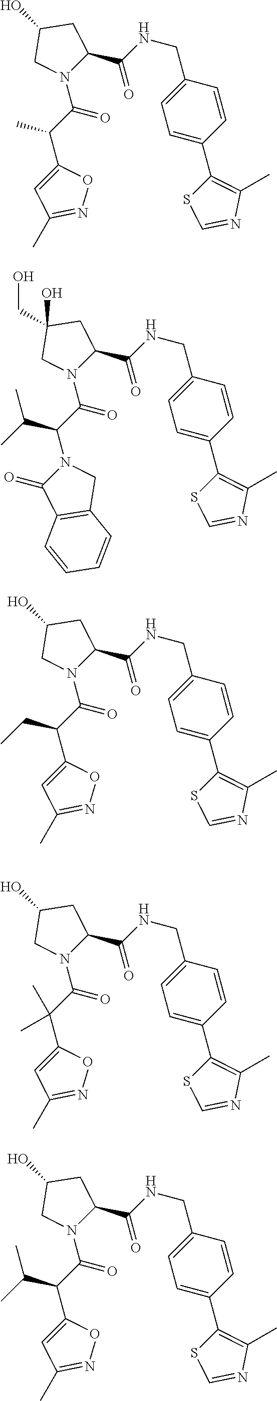

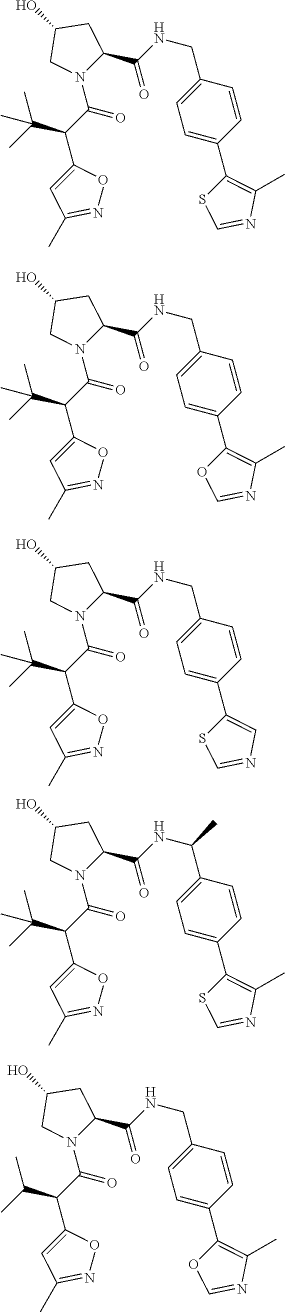

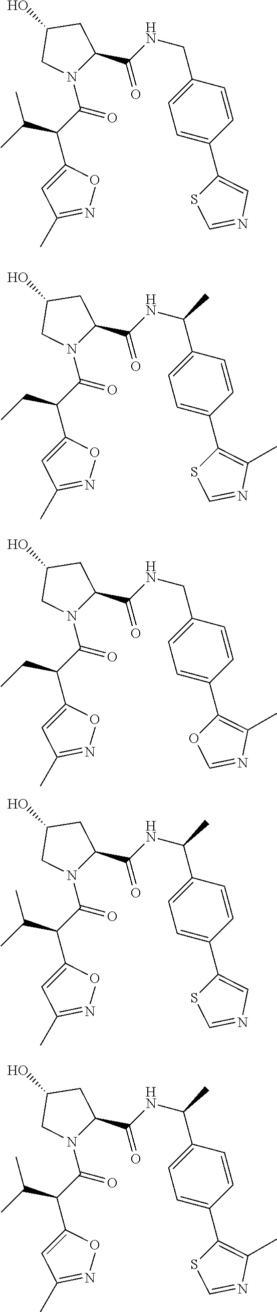

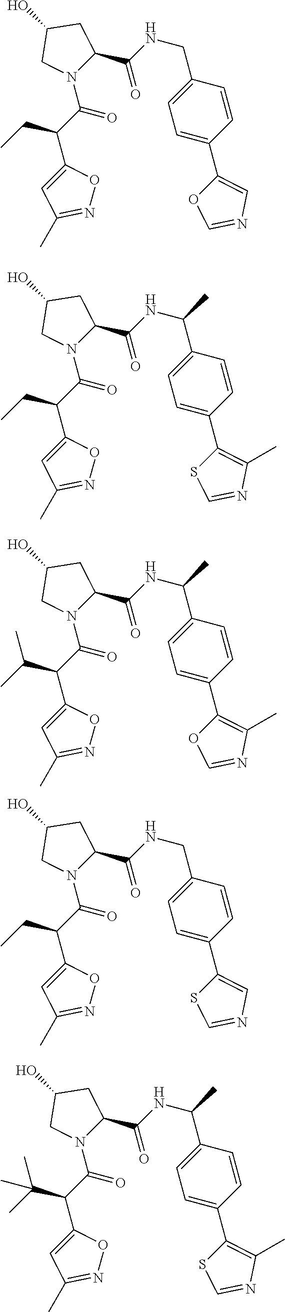

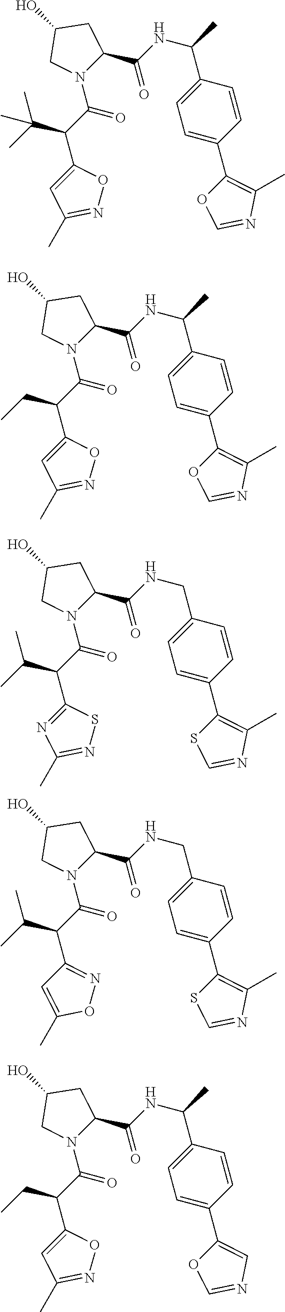

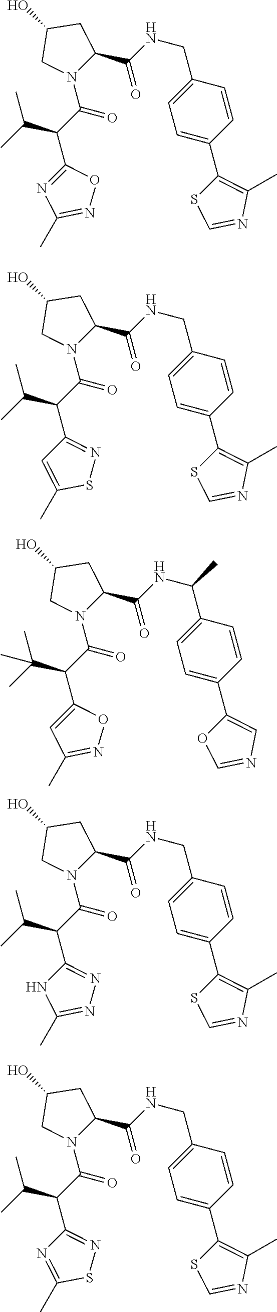

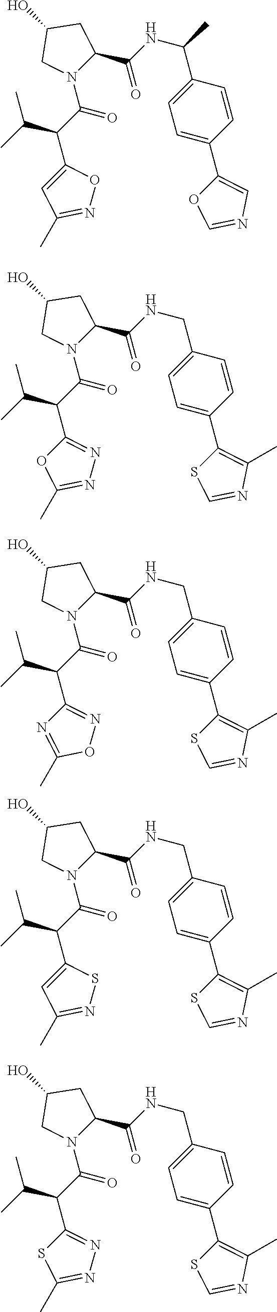

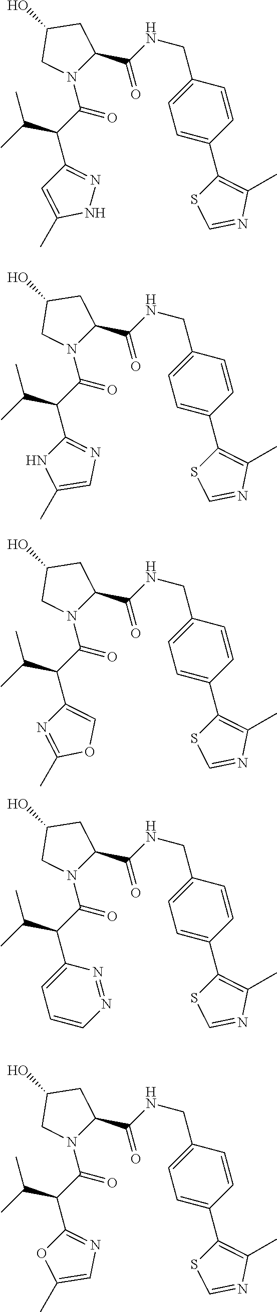

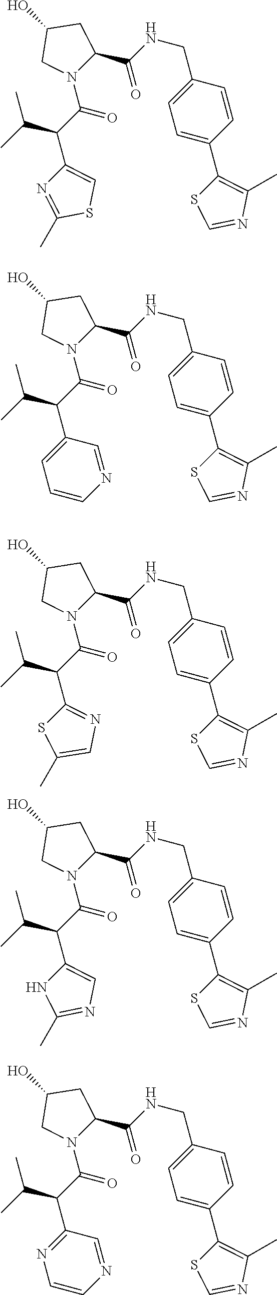

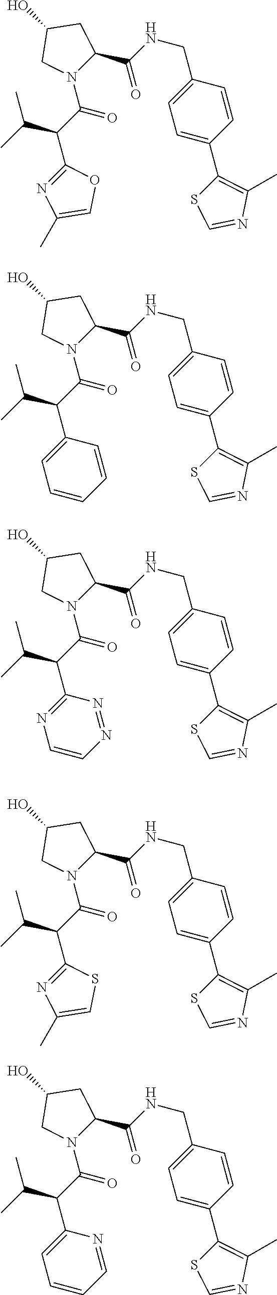

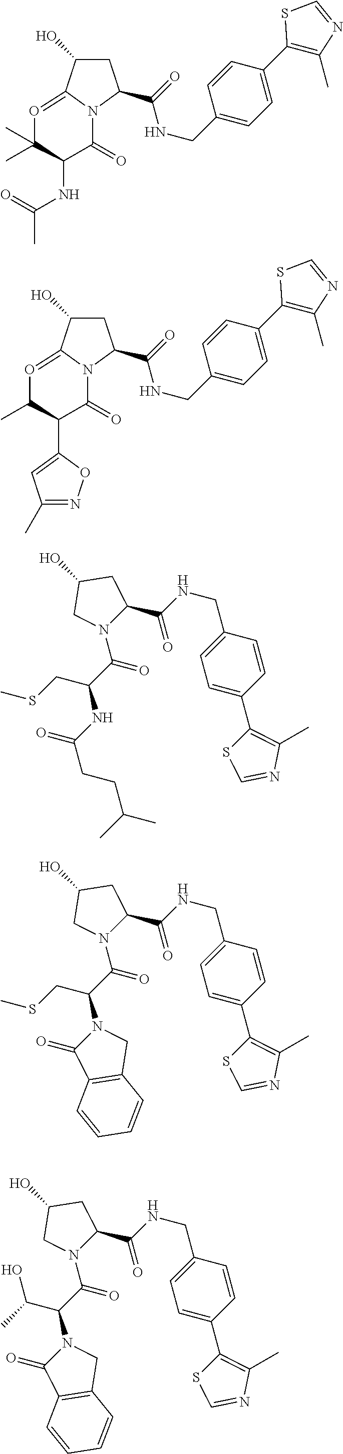

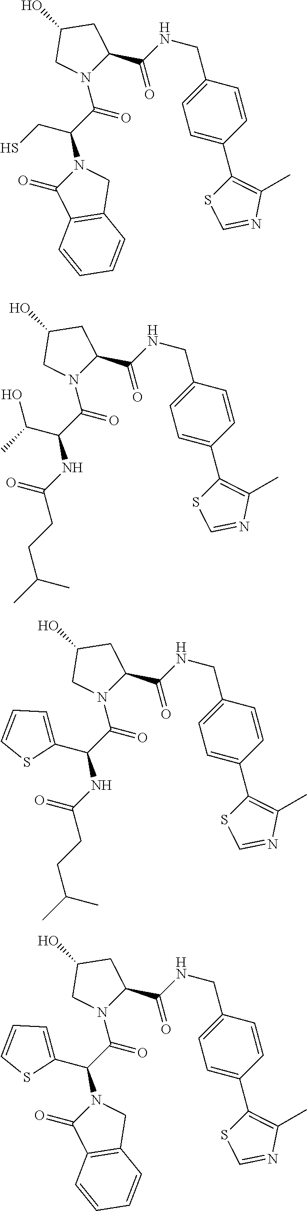

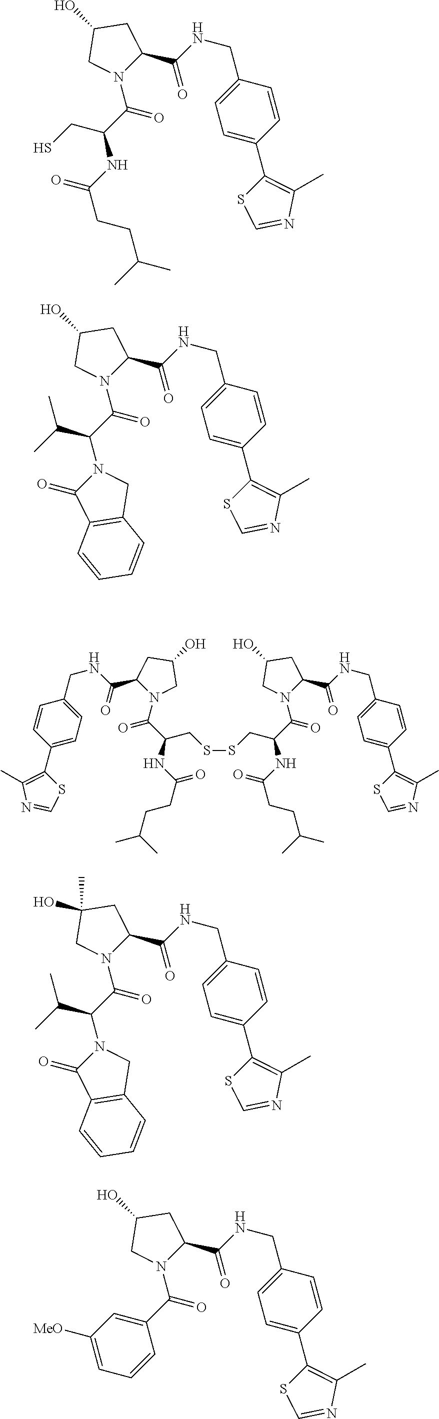

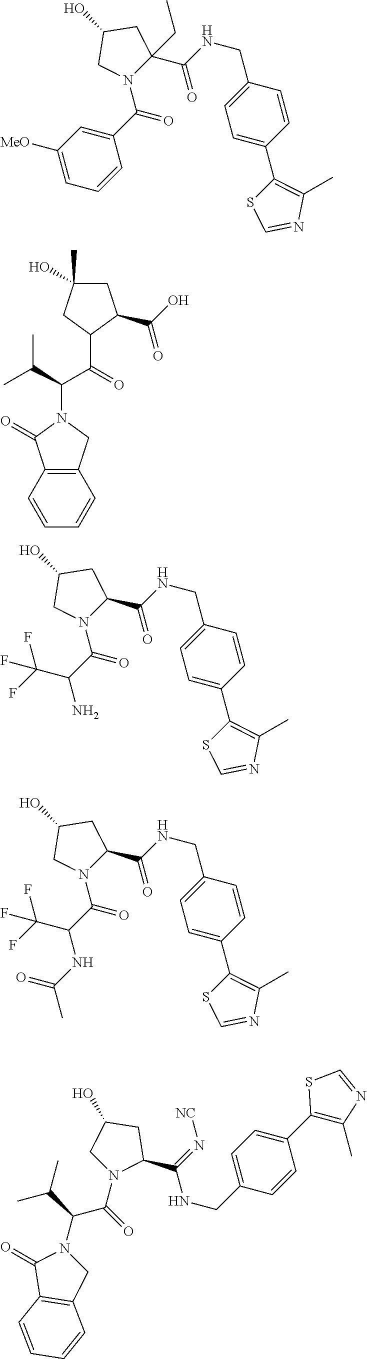

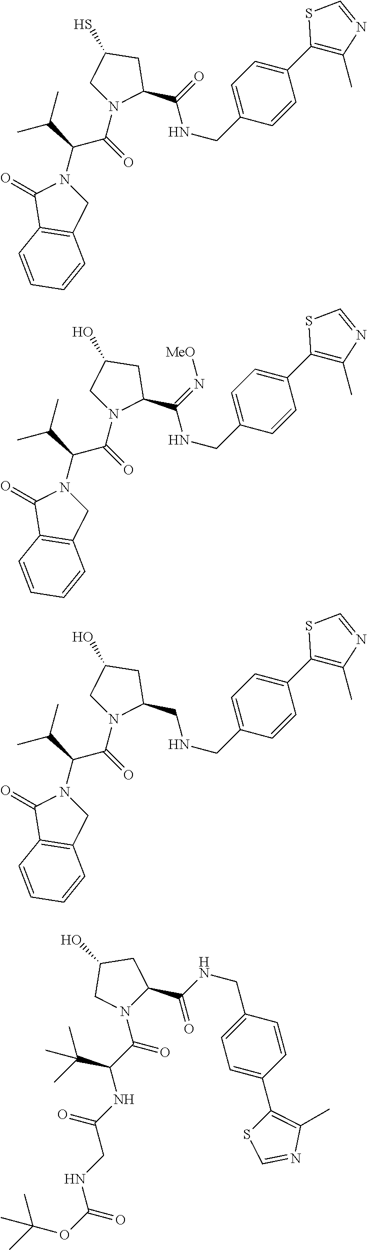

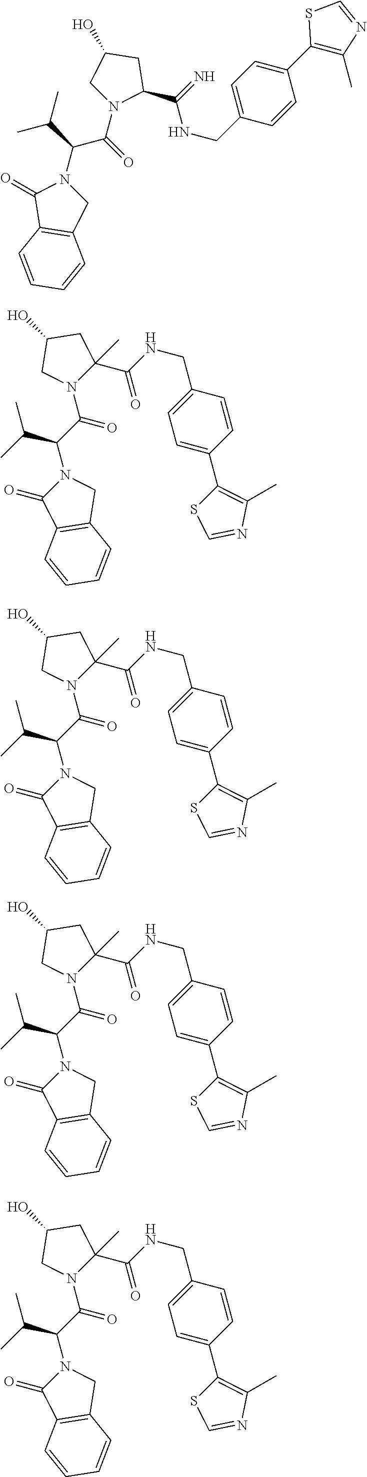

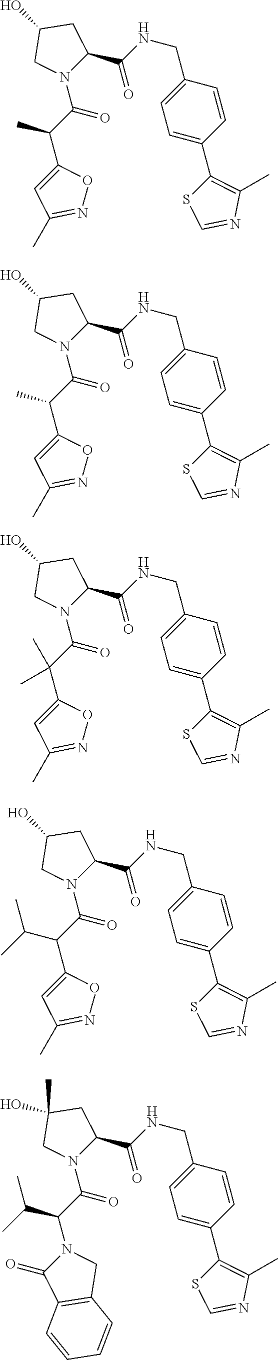

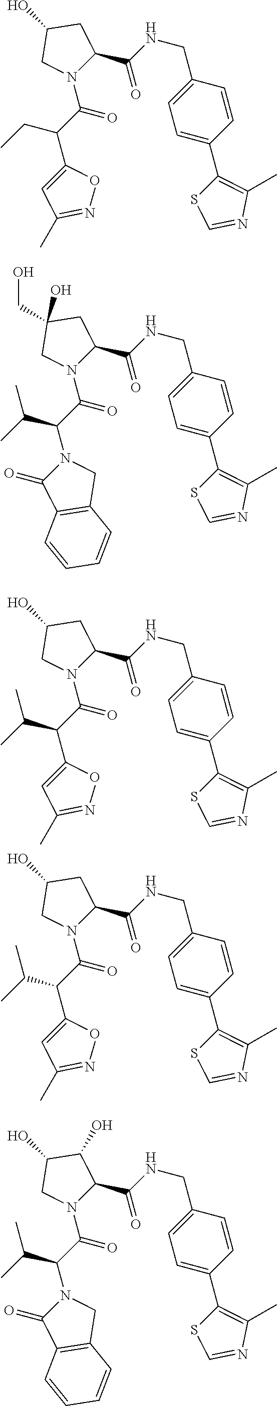

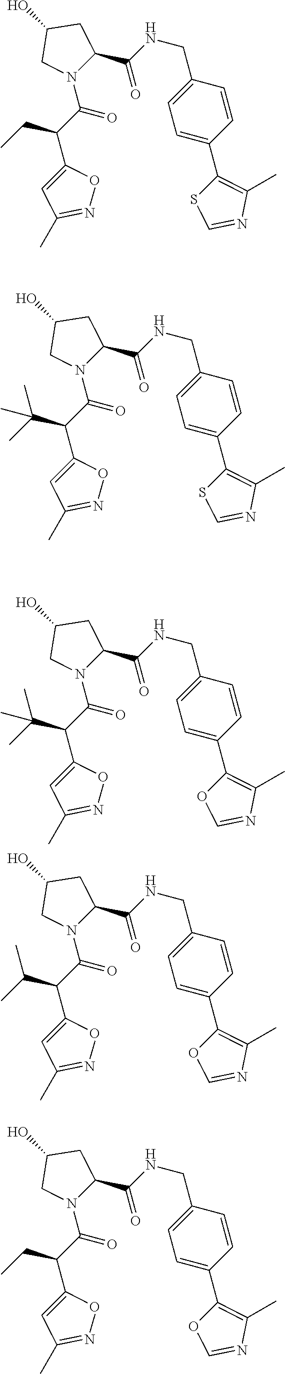

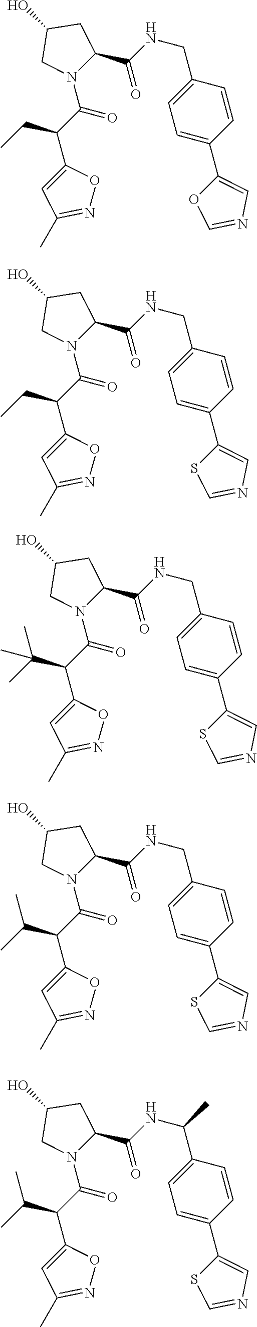

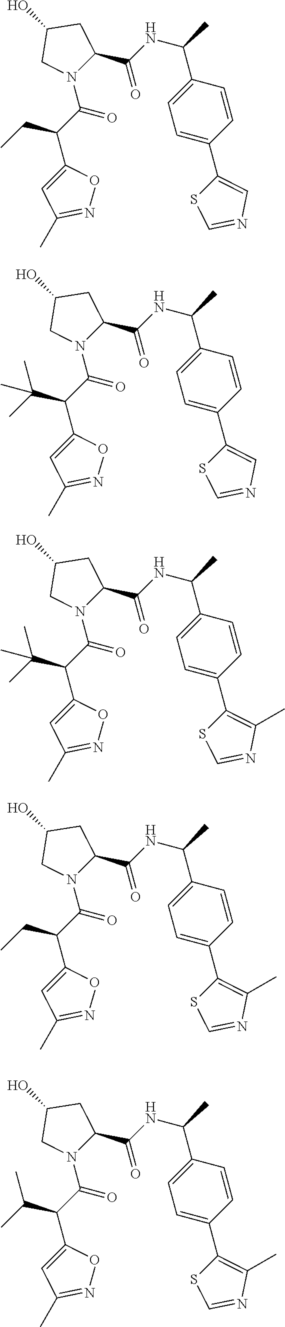

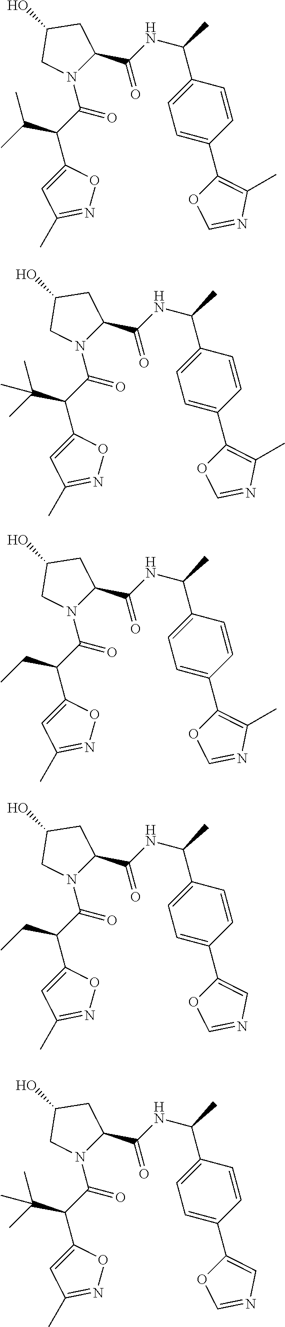

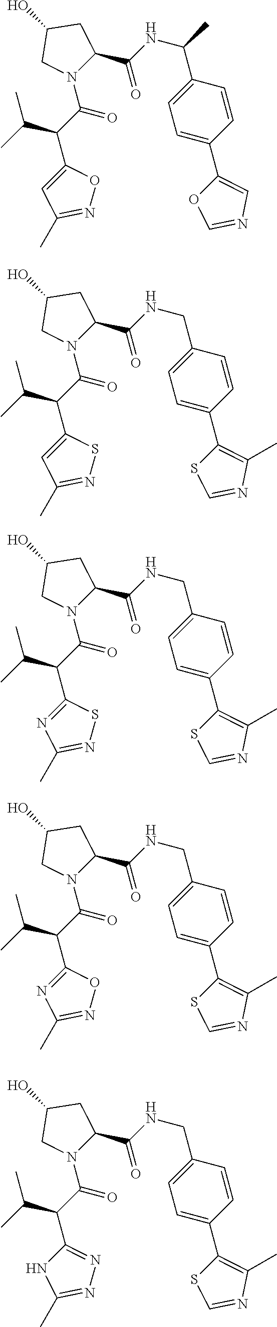

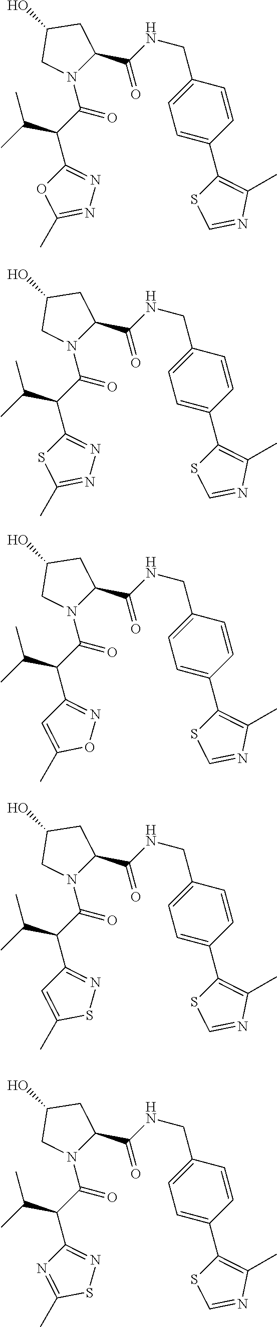

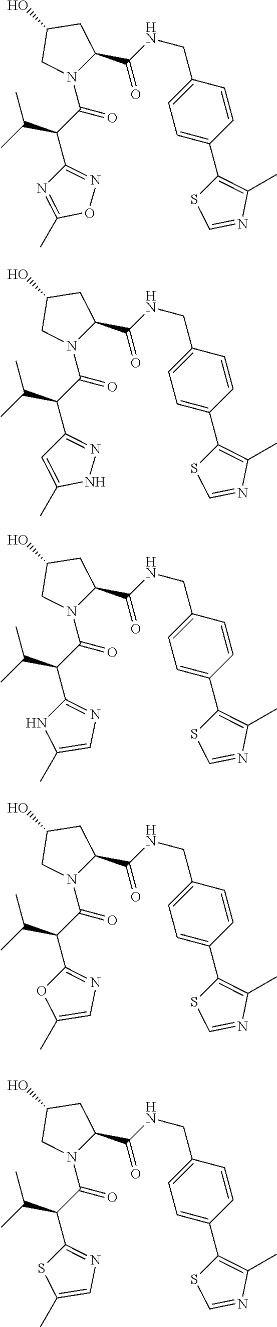

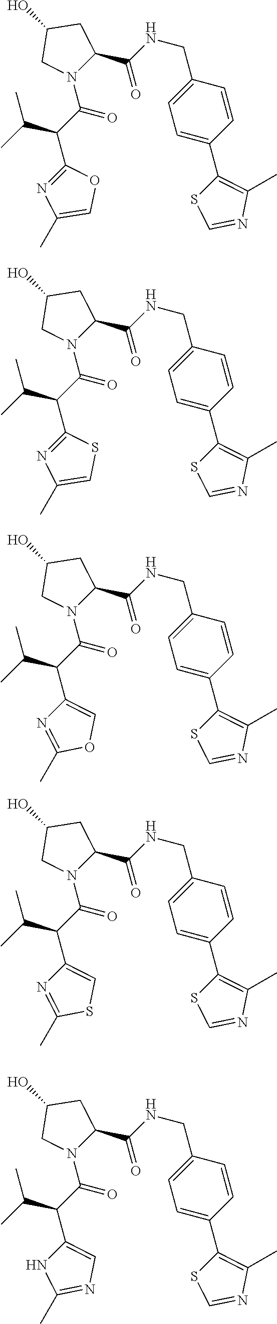

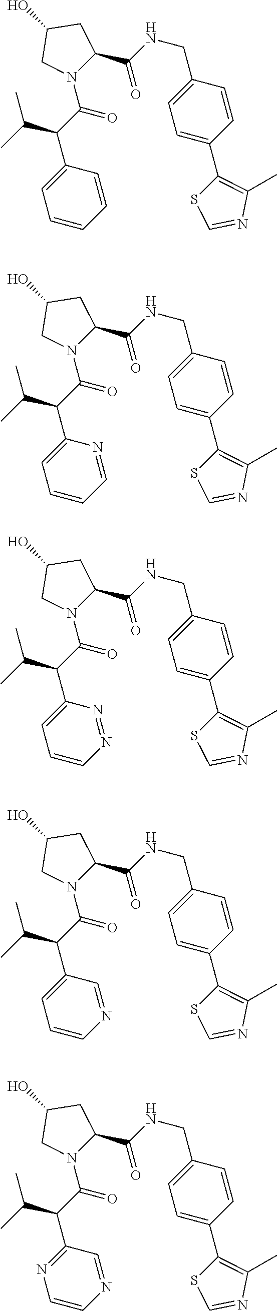

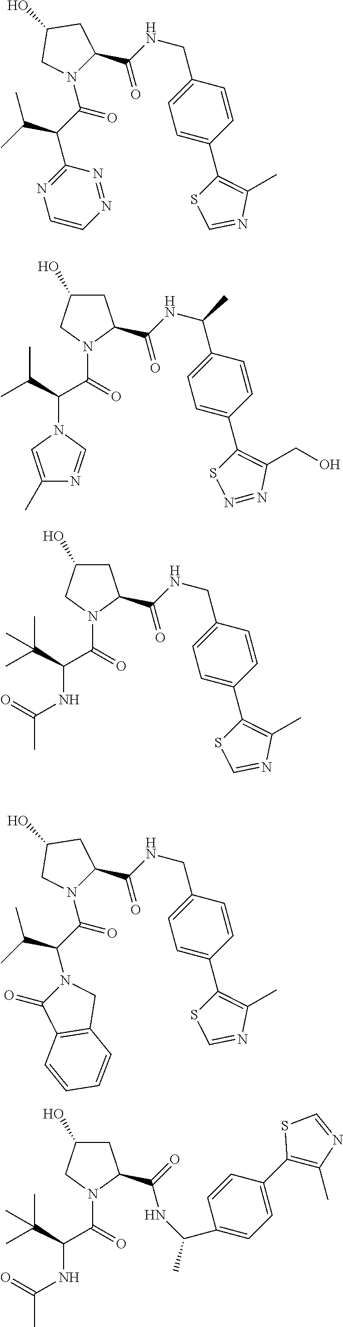

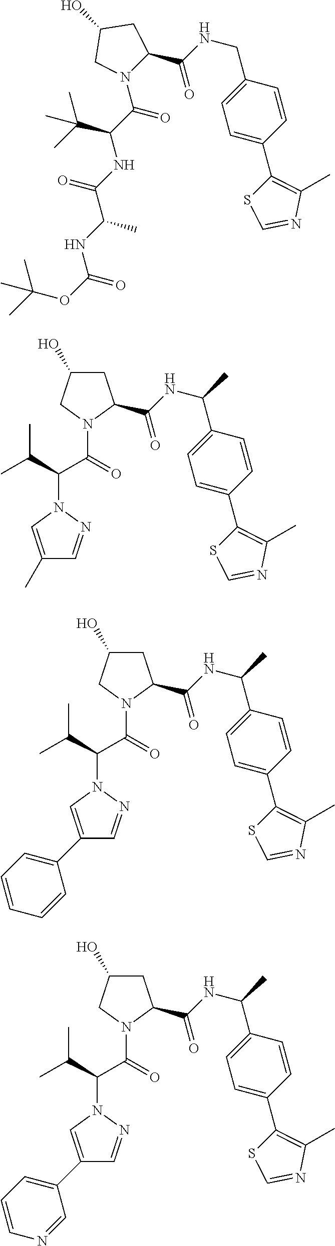

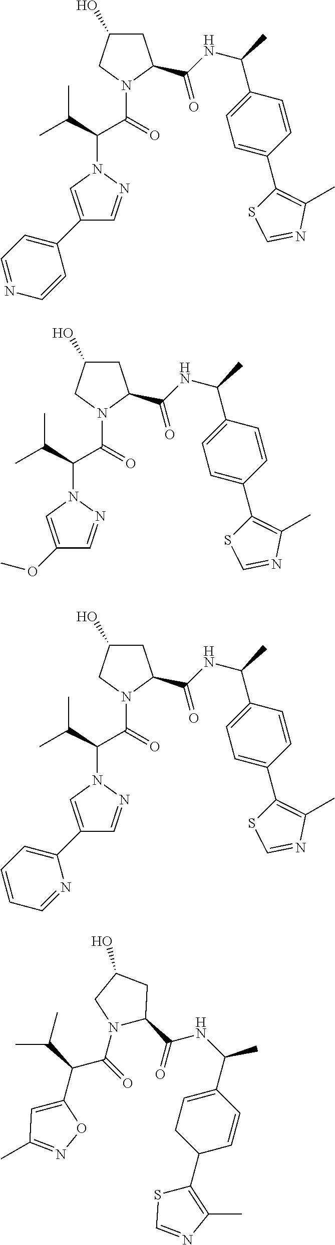

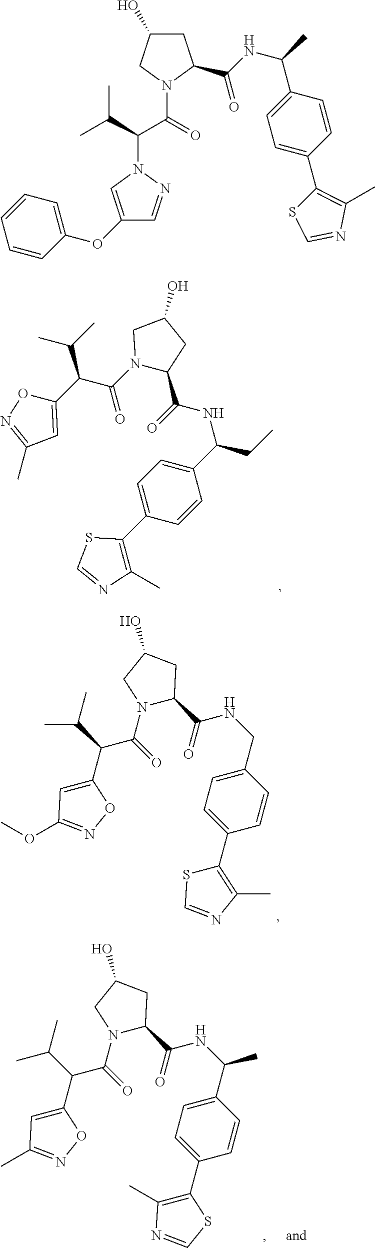

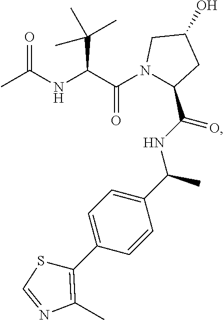 [SMILES: CC(=O)N[C@H](C(=O)N1C[C@H](O)C[C@H]1C(=O)N[C@@H](C)c1ccc(-c2scnc2C)cc1)C(C)(C)C]

wherein the VLM may be connected to a PTM via a linker, as described herein, at any appropriate location, including, e.g., an aryl, heteroary, phenyl, or phenyl of an indole group, optionally via any appropriate functional group, such as an amine, ester, ether, alkyl, or alkoxy.

### Exemplary Linkers

In certain embodiments, the compounds as described herein include one or more PTMs chemically linked or coupled to one or more ULMs (e.g., at least one of CLM, VLM, MLM, ILM, or a combination thereof) via a chemical linker (L). In certain embodiments, the linker group L is a group comprising one or more covalently connected structural units (e.g., -A₁ . . . (A)_{q}- or -(A)_{q}-), wherein A₁ is a group coupled to PTM, and A_{q} is a group coupled to ULM.

In any aspect or embodiment described herein, the linker group L is a bond or a chemical linker group represented by the formula -(A^{L})_{q}-, wherein A is a chemical moiety and q is an integer from 1-100, and wherein L is covalently bound to the PTM and the ULM, and provides for sufficient binding of the PTM to the protein target and the ULM to an E3 ubiquitin ligase to result in target protein ubiquitination.

In certain embodiments, the linker group L is -(A)_{q}-:
(A)_{q} is a group which is connected to at least one of a ULM moiety, (such as CLM or VLM), PTM moiety, or both; and
q of the linker is an integer greater than or equal to 1;
each A is independently selected from the group consisting of, a bond, CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₅₋₁₃ spirocycloalkyl optionally substituted with 0-9 R^{L1} and/or R^{L2} groups, C₃₋₁₁heterocyclyl optionally substituted with 0-6 R^{L1} and/or R^{L2} groups, C₅₋₁₃ spiroheterocycloalkyl optionally substituted with 0-8 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R^{L5} groups; and
R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NH SO₂NH(C₁₋₈alkyl), NH SO₂N(C₁₋₈alkyl)₂, NH SO₂NH₂.

In certain embodiments, q of the linker is an integer greater than or equal to 0. In certain embodiments, q is an integer greater than or equal to 1.

In certain embodiments, e.g., where q of the linker is greater than 2, A_{q} is a group which is connected to ULM, and A₁ and A_{q} are connected via structural units of the linker (L).

In certain embodiments, e.g., where q of the linker is 2, A_{q} is a group which is connected to A₁ and to a ULM.

In certain embodiments, e.g., where q of the linker is 1, the structure of the linker group L is -A₁-, and A₁ is a group which is connected to a ULM moiety and a PTM moiety.

In other embodiments, the linker is

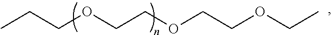

wherein n is an integer from 0 to 10.

In yet other embodiments, the linker is

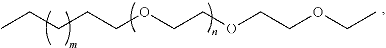

wherein n is an integer from 0 to 10, and m is an integer from 2 to 10.

In still other embodiments, the linker is

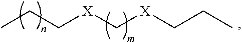

wherein n is an integer from 0 to 10, m is an integer from 0 to 10, and X is independently O or CH₂.

In certain embodiments, the linker (L) comprises a group represented by a general structure selected from the group consisting of:
—NR(CH₂)ₙ-(lower alkyl)-, —NR(CH₂)ₙ-(lower alkoxyl)-, —NR(CH₂)ₙ-(lower alkoxyl)-OCH₂—, —NR(CH₂)ₙ-(lower alkoxyl)-(lower alkyl)-OCH₂—, —NR(CH₂)ₙ-(cycloalkyl)-(lower alkyl)-OCH₂—, —NR(CH₂)ₙ-(hetero cycloalkyl)-, —NR(CH₂CH₂O)ₙ-(lower alkyl)-O—CH₂—, —NR(CH₂CH₂O)ₙ-(hetero cycloalkyl)-O—CH₂—, —NR(CH₂CH₂O)ₙ-Aryl-O—CH₂—, —NR(CH₂CH₂O)ₙ-(hetero aryl)-O—CH₂—, —NR(CH₂CH₂O)ₙ-(cyclo alkyl)-O-(hetero aryl)-O—CH₂—, —NR(CH₂CH₂O)ₙ-(cyclo alkyl)-O-Aryl-O—CH₂—, —NR(CH₂CH₂O)ₙ-(lower alkyl)-NH-Aryl-O—CH₂—, —NR(CH₂CH₂O)ₙ-(lower alkyl)-O-Aryl-CH₂, —NR(CH₂CH₂O)ₙ-cycloalkyl-O-Aryl-, —NR(CH₂CH₂O)ₙ-cycloalkyl-O-(heteroaryl)l-, —NR(CH₂CH₂)ₙ-(cycloalkyl)-O-(heterocycle)-CH₂, —NR(CH₂CH₂)ₙ-(heterocycle)-(heterocycle)-CH₂, —N(R1R2)-(heterocycle)-CH₂; where
   n of the linker can be 0 to 10;
   R of the linker can be H, lower alkyl;
   R1 and R2 of the linker can form a ring with the connecting N.

In any aspect or embodiment described herein, the linker is an poly(alkylene) glycol (e.g., polyoxymethylene, ethylene glycol, propylene glycol, or combinations thereof) or of the chemical structure —[O(CH₂)ₙ]ₘ—, wherein each n is independently 1, 2, 3, 4, 5, 6, 7 or 8, and m is an integer from 1-100 (e.g., 1, 2, 3, 4, 5, 6, 7, 8, 9, or 10).

In any aspect or embodiment described herein, the linker is selected from:

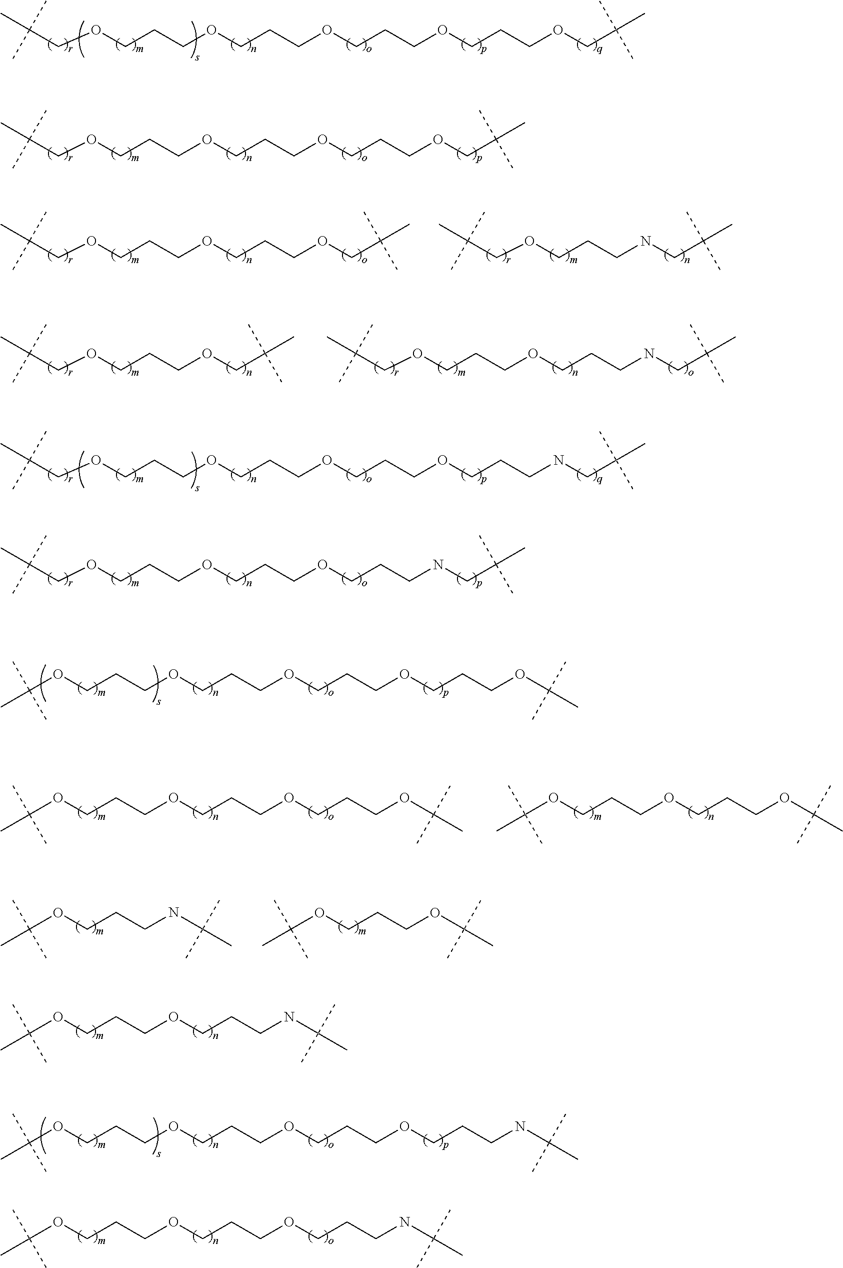

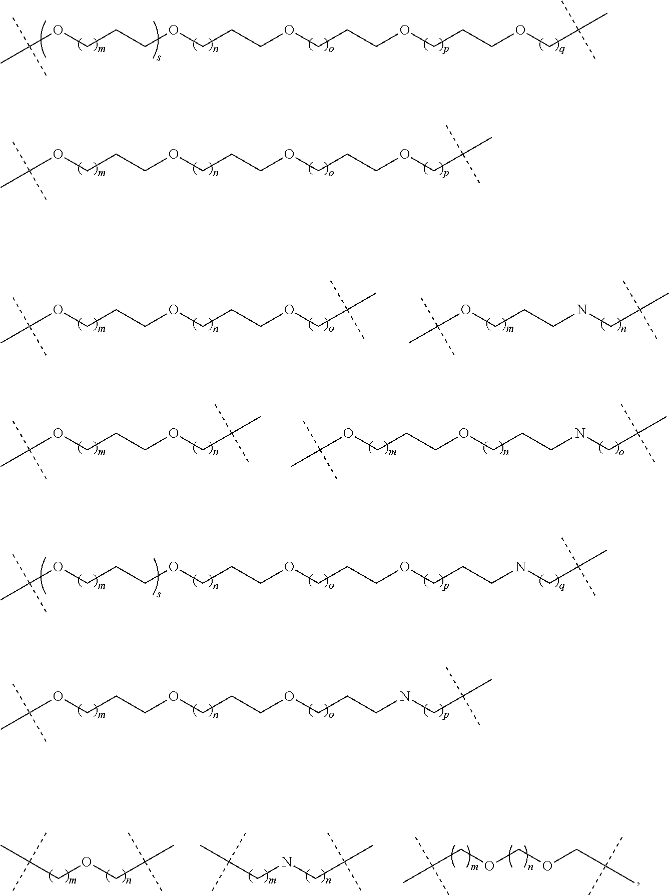

wherein each m, n, o, p, q, r, and s is independently 0, 1, 2, 3, 4, 5, 6, 7, 8, 9, 10, 11, 12, 13, 14, 15, 16, 17, 18, 19, or 20.

In some embodiments, the linker (L) includes at least one of:

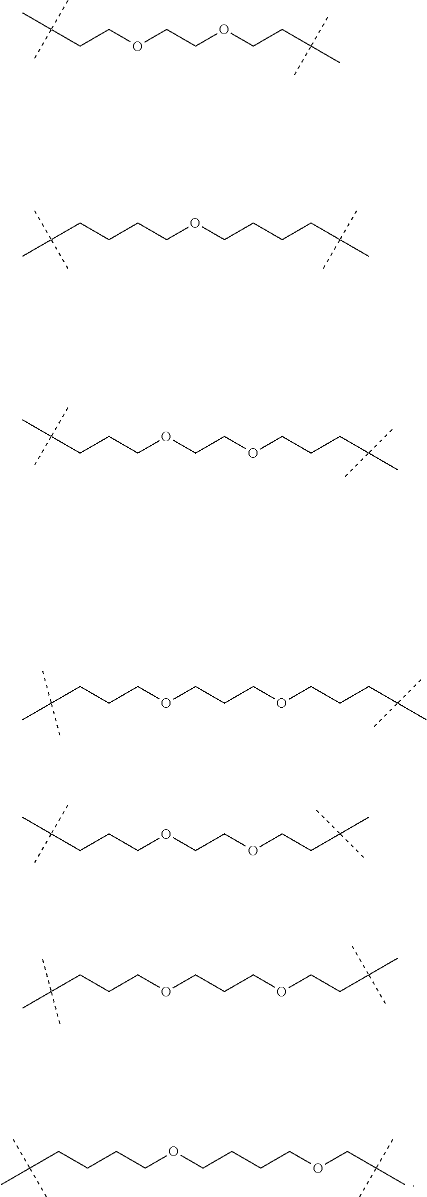

In certain embodiments, the linker (L) comprises a group represented by a general structure selected from the group consisting of:
—N(R)—(CH2)ₙ—O(CH2)ₙ—O(CH2)ₒ—O(CH2)ₚ—O(CH2)_{q}—O(CH2)ᵣ-OCH2-, —O—(CH2)ₘ—O(CH2)ₙ—O(CH2)ₒ—O(CH2)ₚ—O(CH2)_{q}—O(CH2)ᵣ-OCH2-, —O—(CH2)ₘ—O(CH2)ₙ—O(CH2)ₒ—O(CH2)ₚ—O(CH2)_{q}—O(CH2)ᵣ—O—;
—N(R)—(CH2)ₘ—O(CH2)ₙ—O(CH2)ₒ—O(CH2)ₚ—O(CH2)_{q}—O(CH2)ᵣ—O—;
—(CH2)ₘ—O(CH2)ₙ—O(CH2)ₒ—O(CH2)ₚ—O(CH2)_{q}—O(CH2)ᵣ—O—;
—(CH2)ₘ—O(CH2)ₙ—O(CH2)ₒ—O(CH2)ₚ—O(CH2)_{q}—O(CH2)ᵣ-OCH2-;

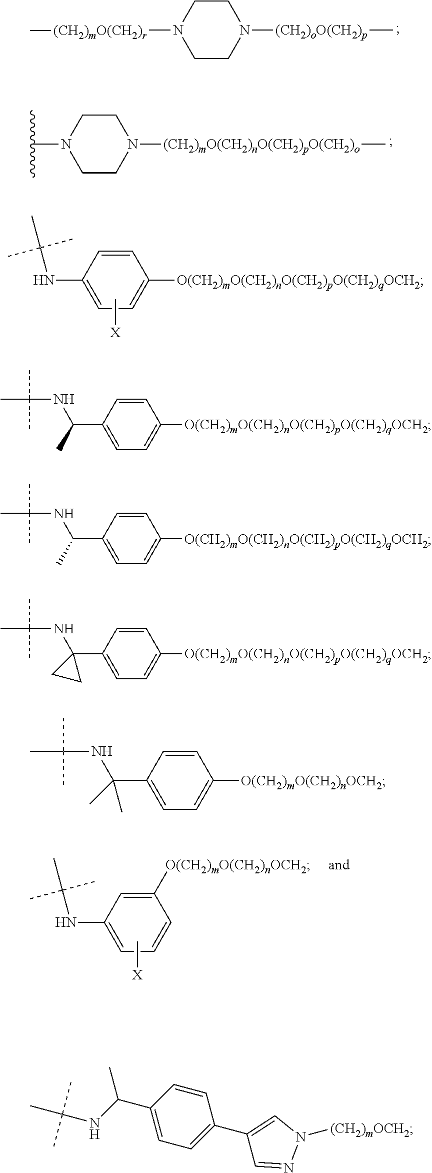

wherein

m, n, o, p, q, and r of the linker are independently 0, 1, 2, 3, 4, 5, 6, 7, 8, 9, 10, 11, 12, 13, 14, 15, 16, 17, 18, 19, 20;

when the number is zero, there is no N—O or O—O bond

R of the linker is H, methyl and ethyl;

X of the linker is H and F

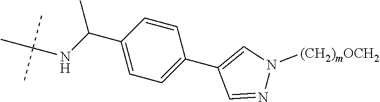

where m of the linker can be 2, 3, 4, 5

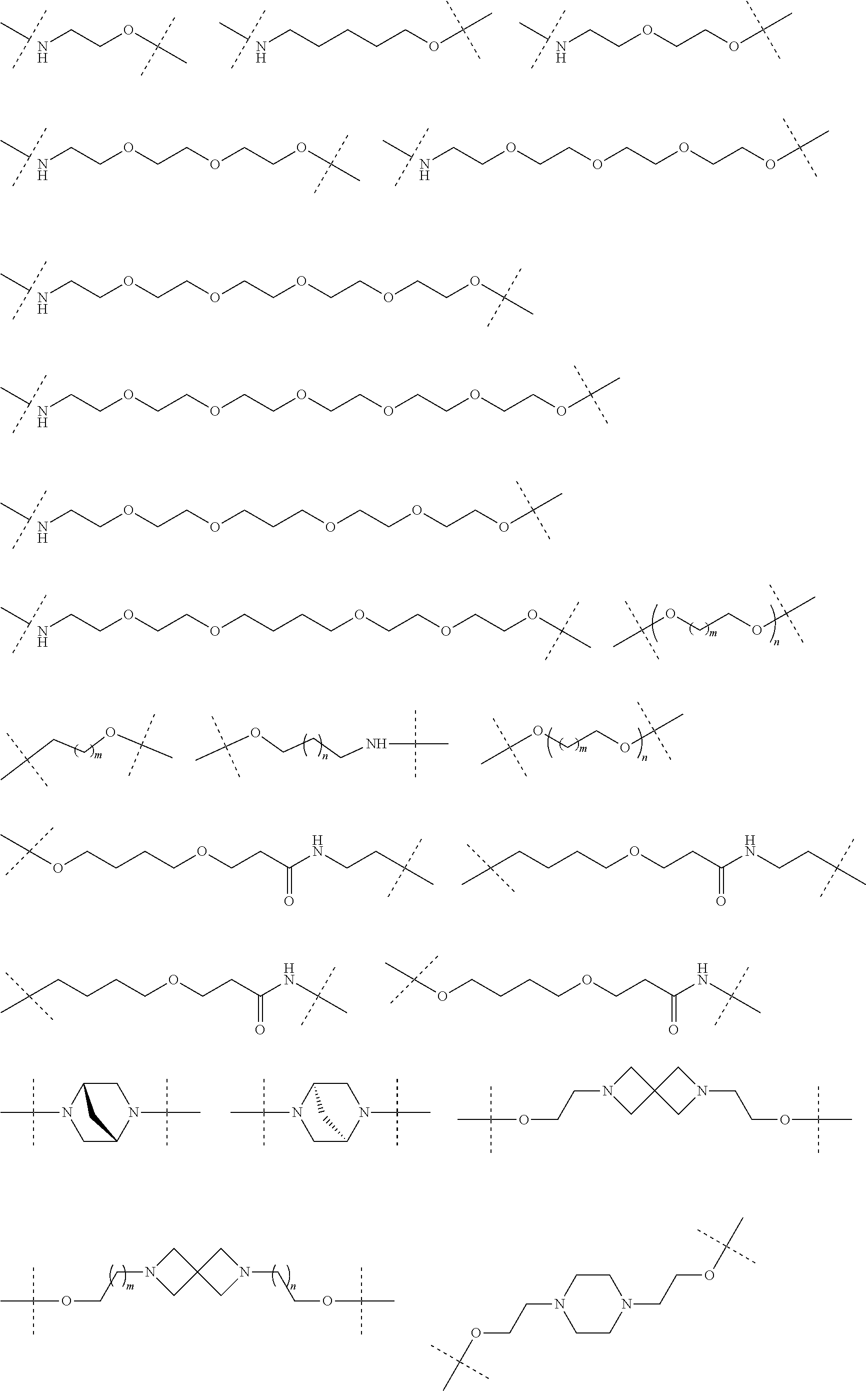

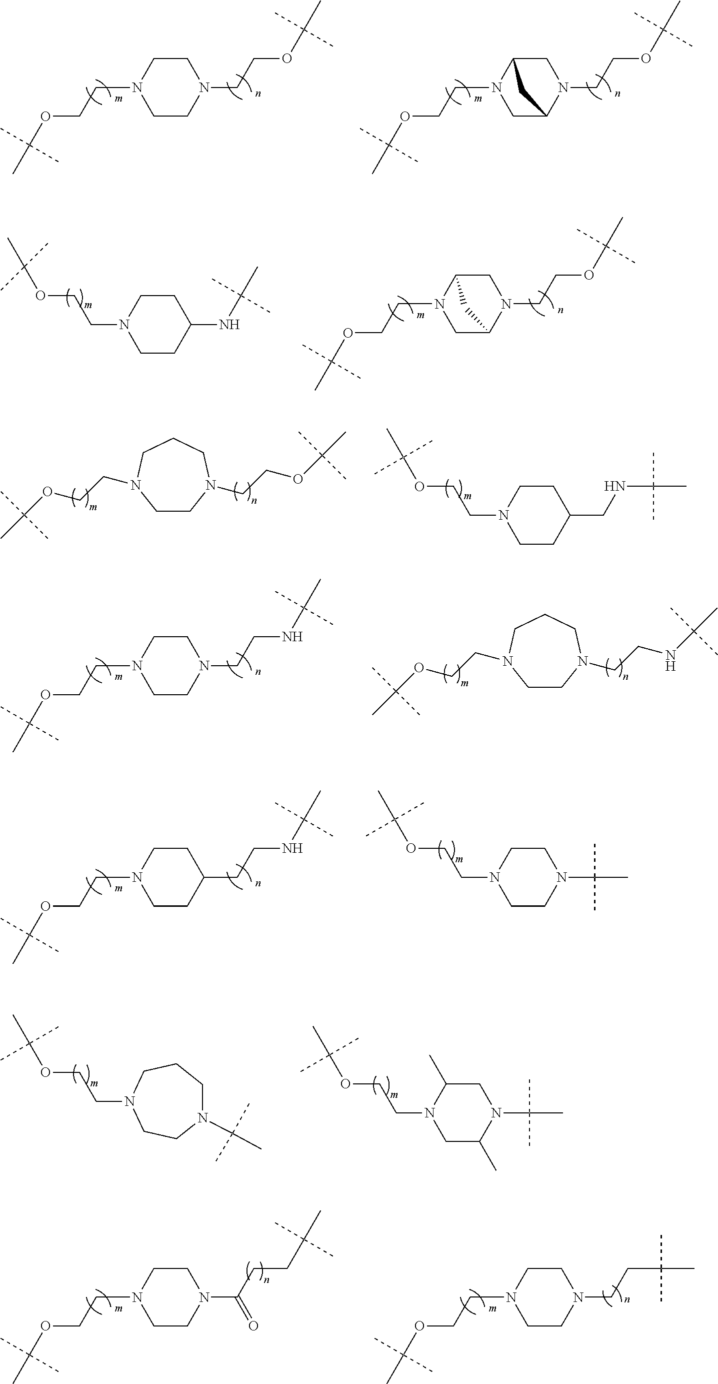

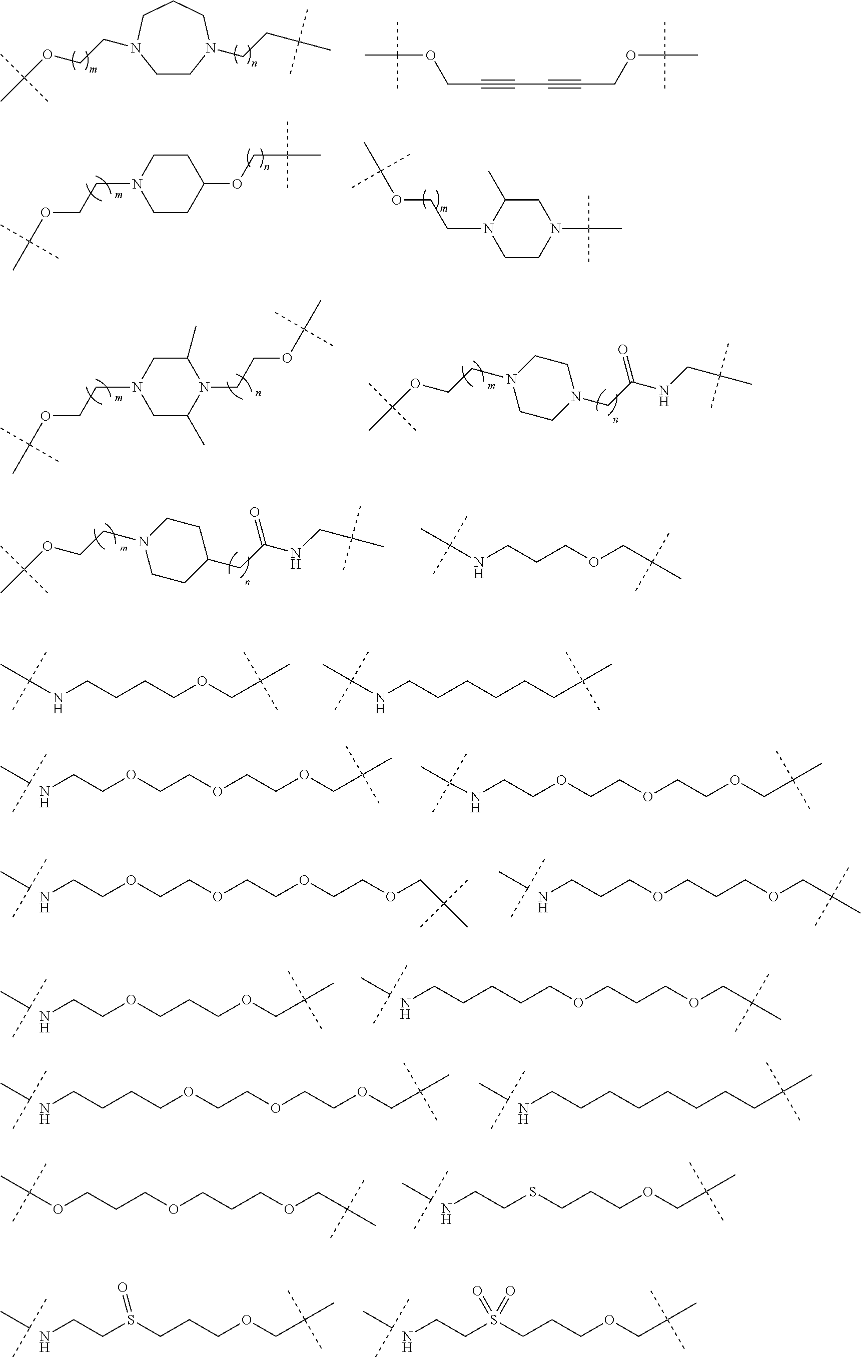

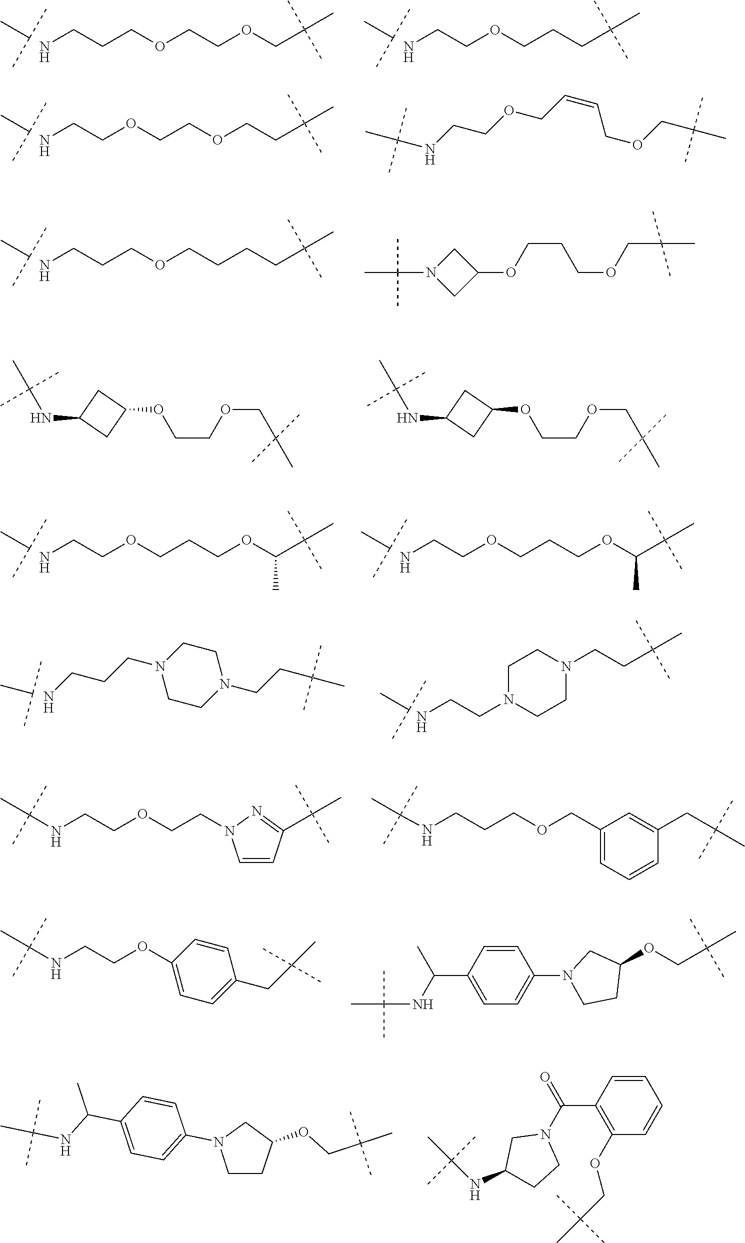

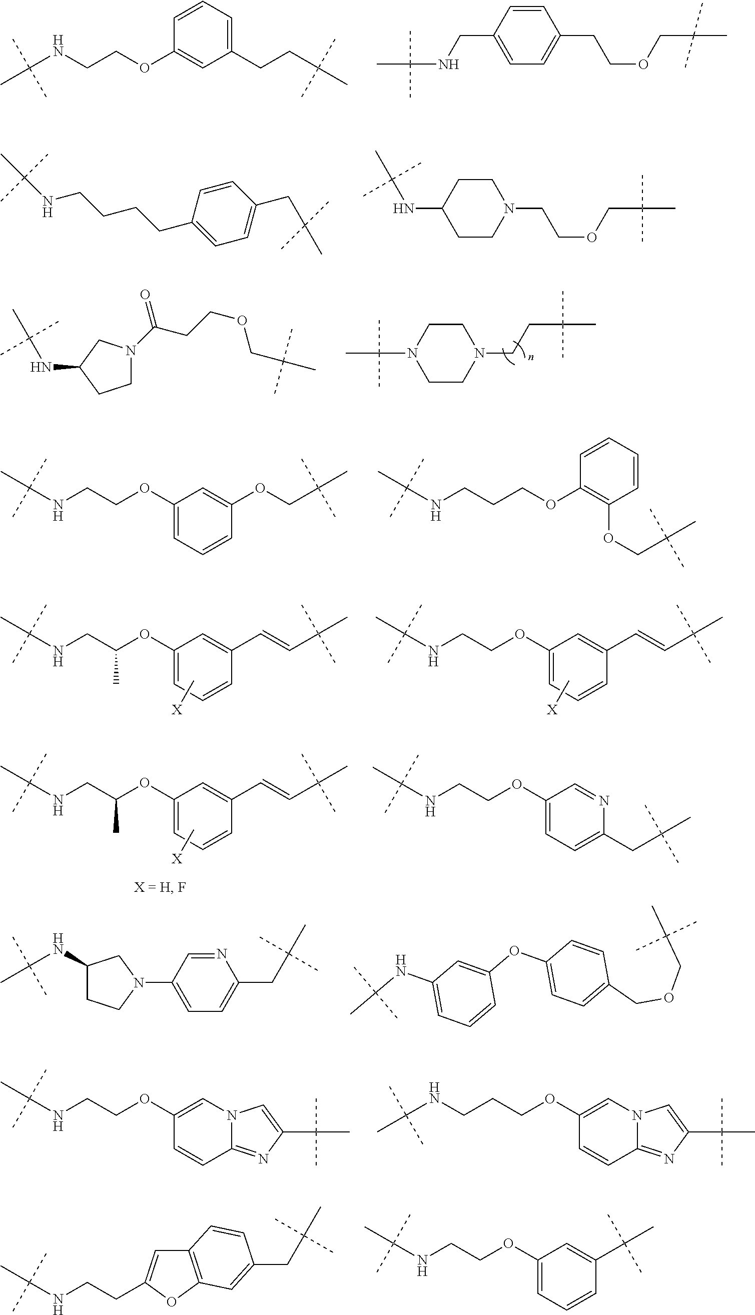

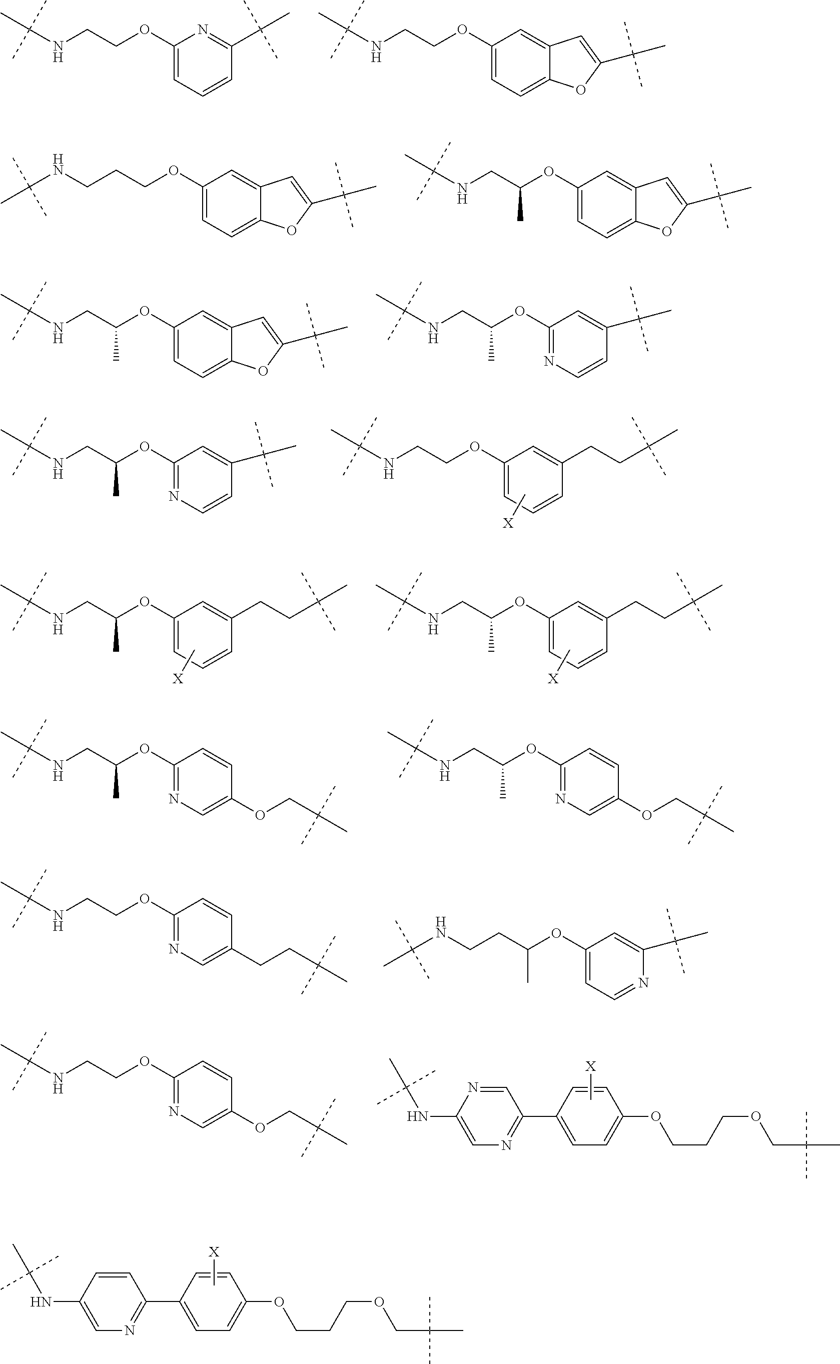

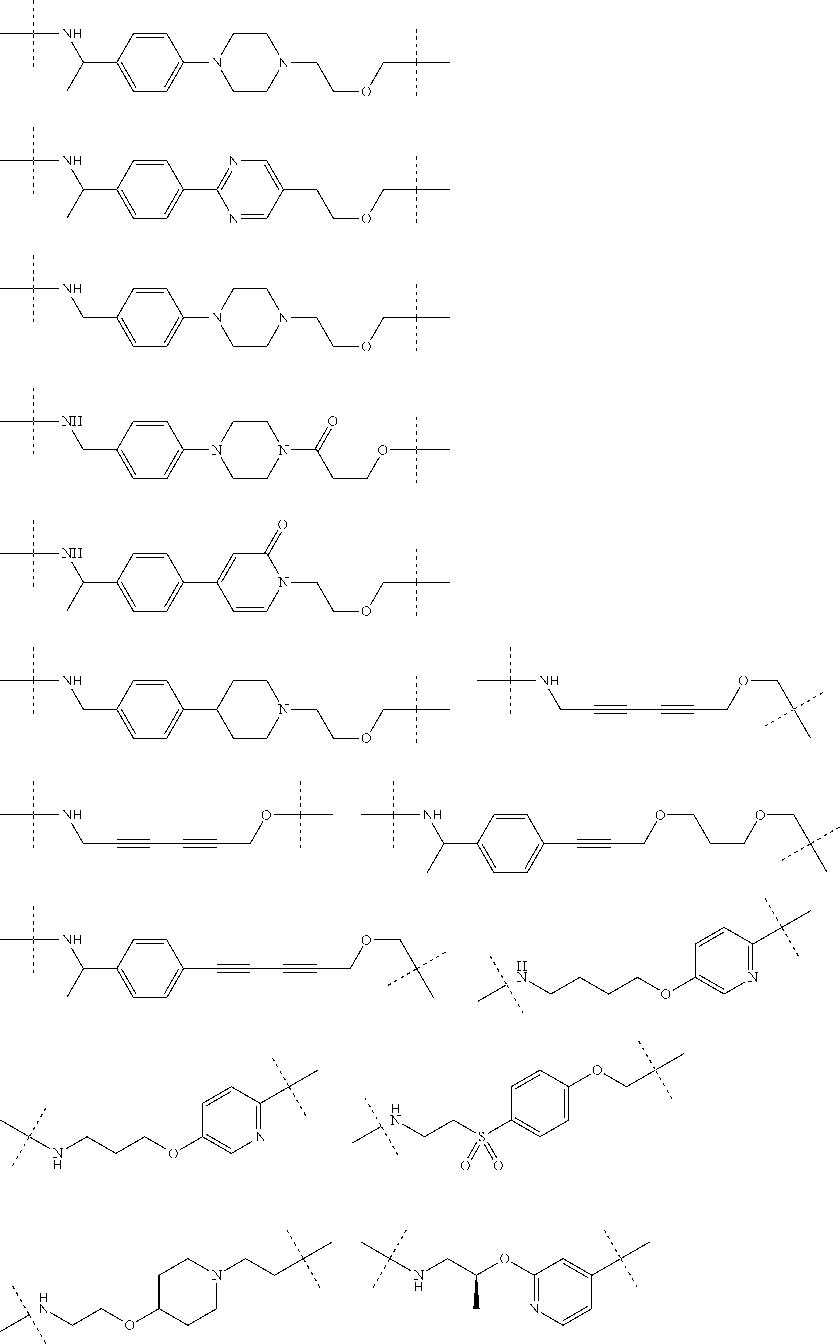

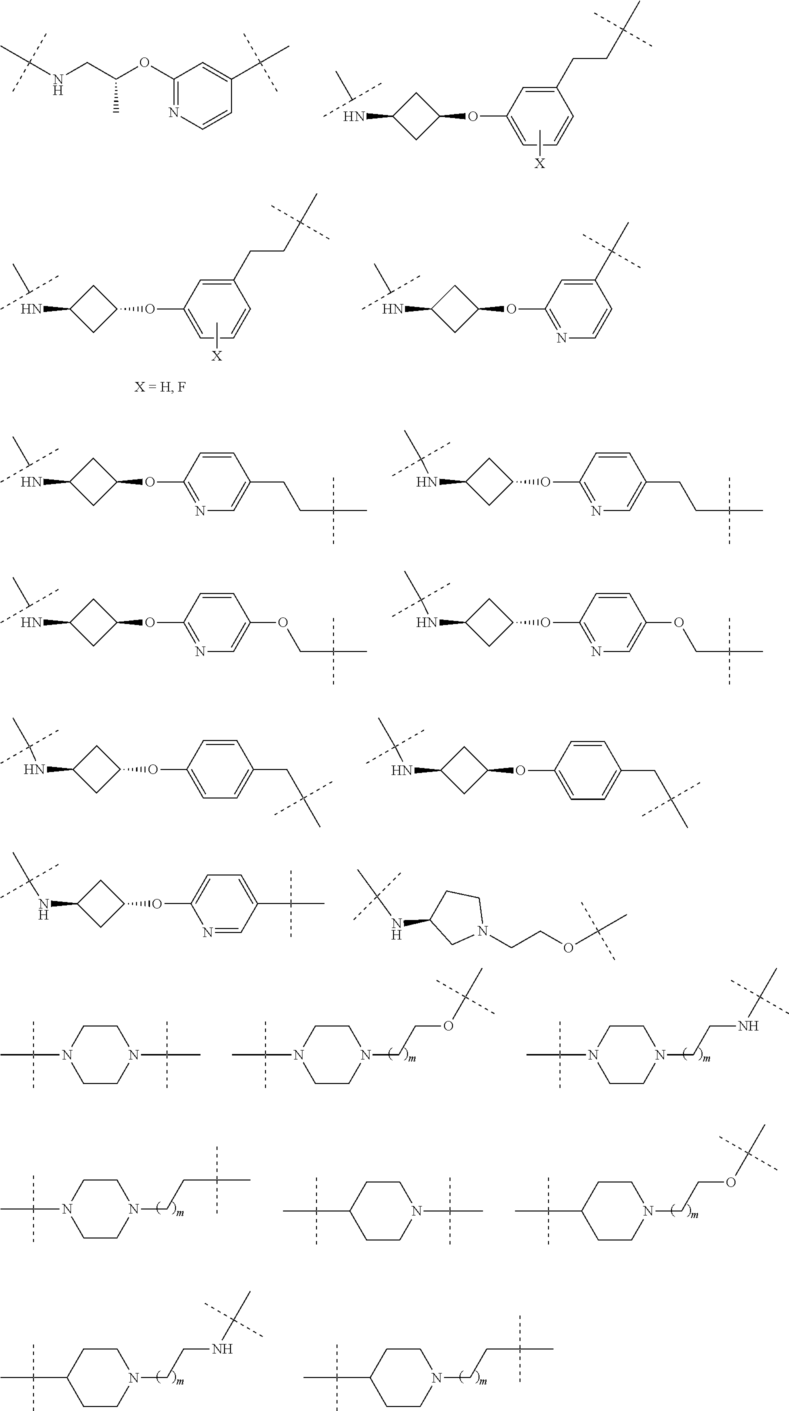

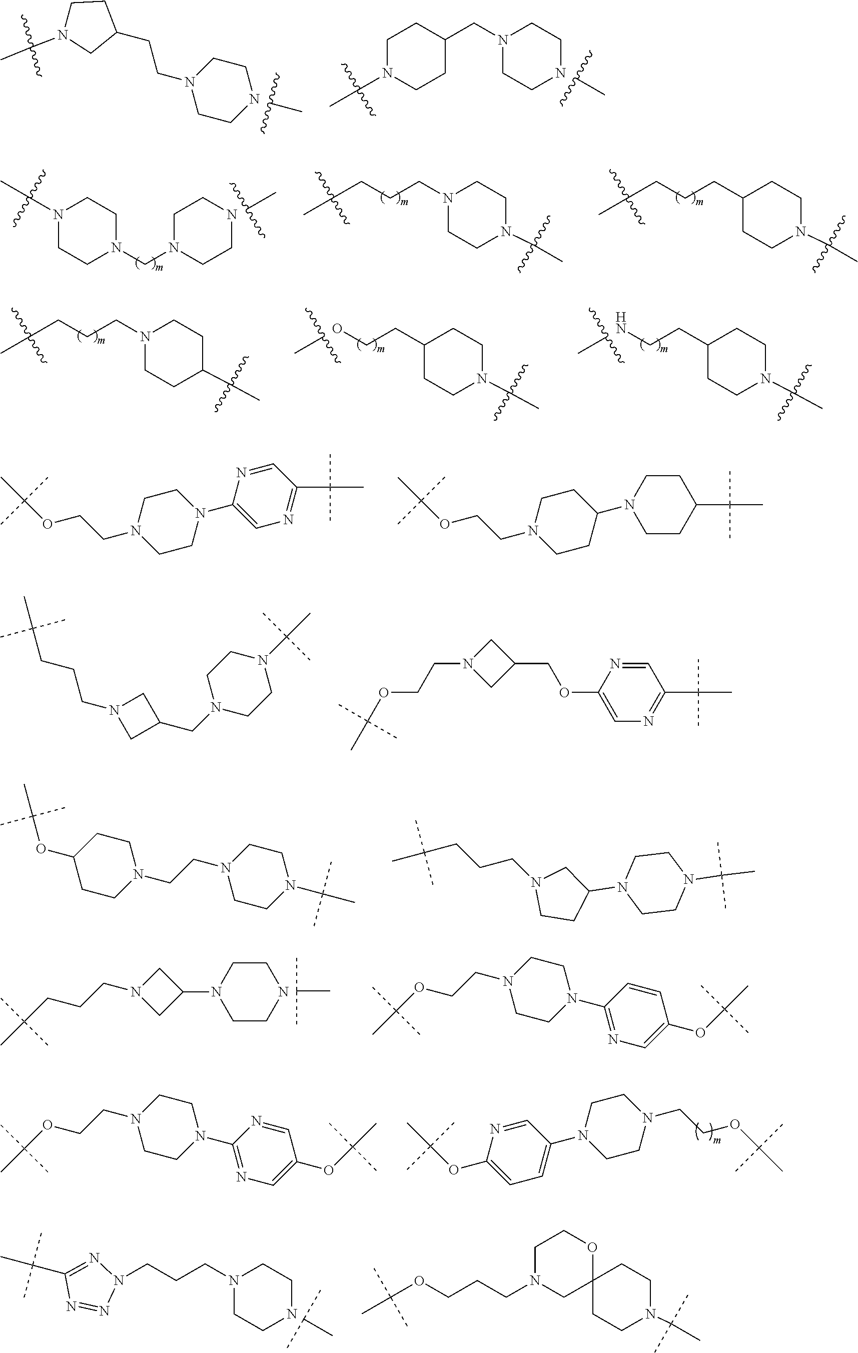

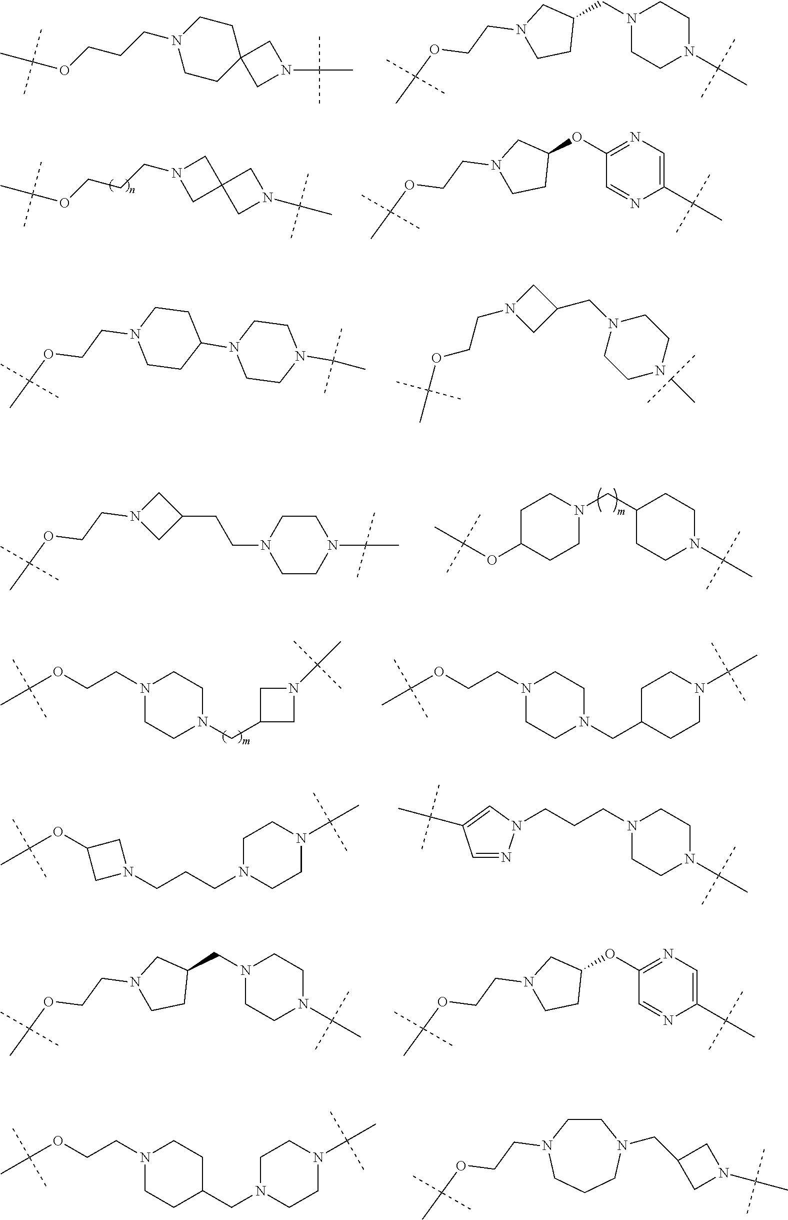

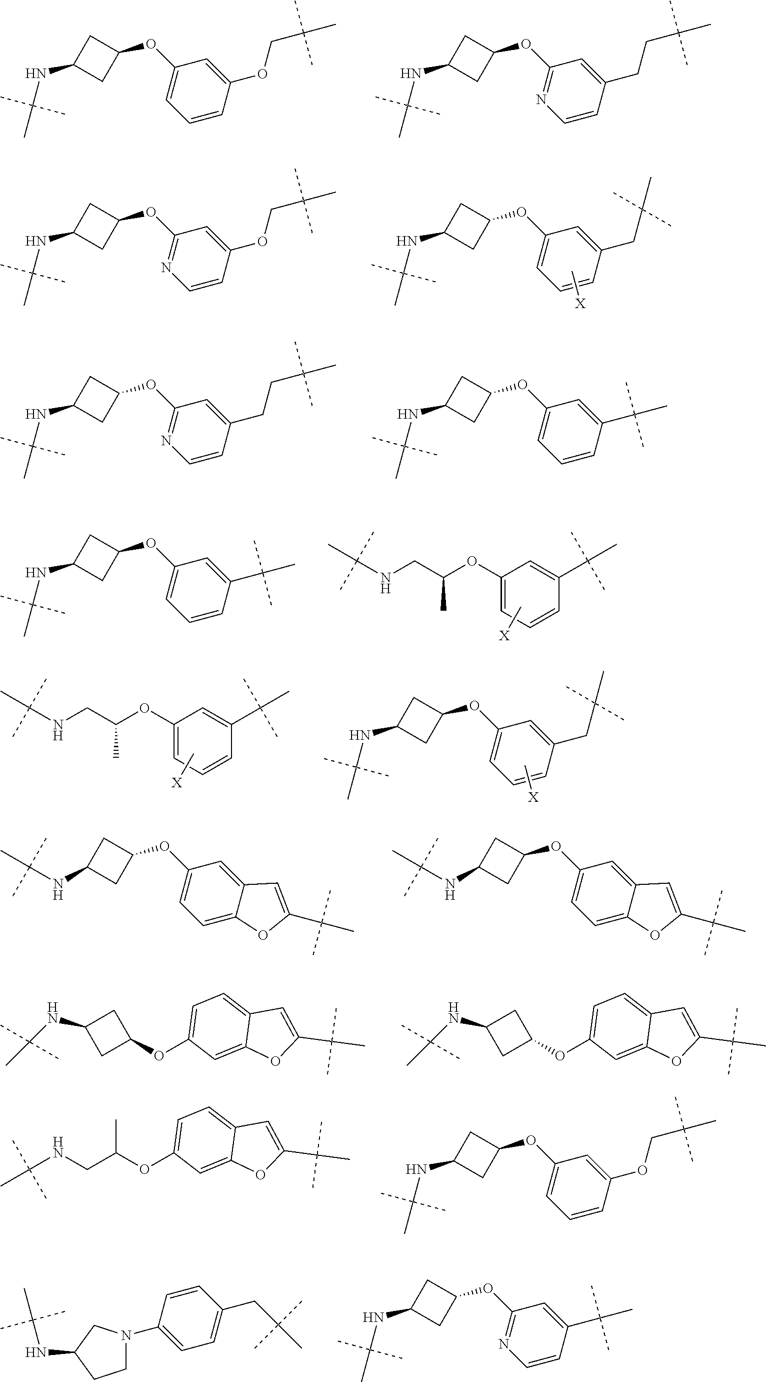

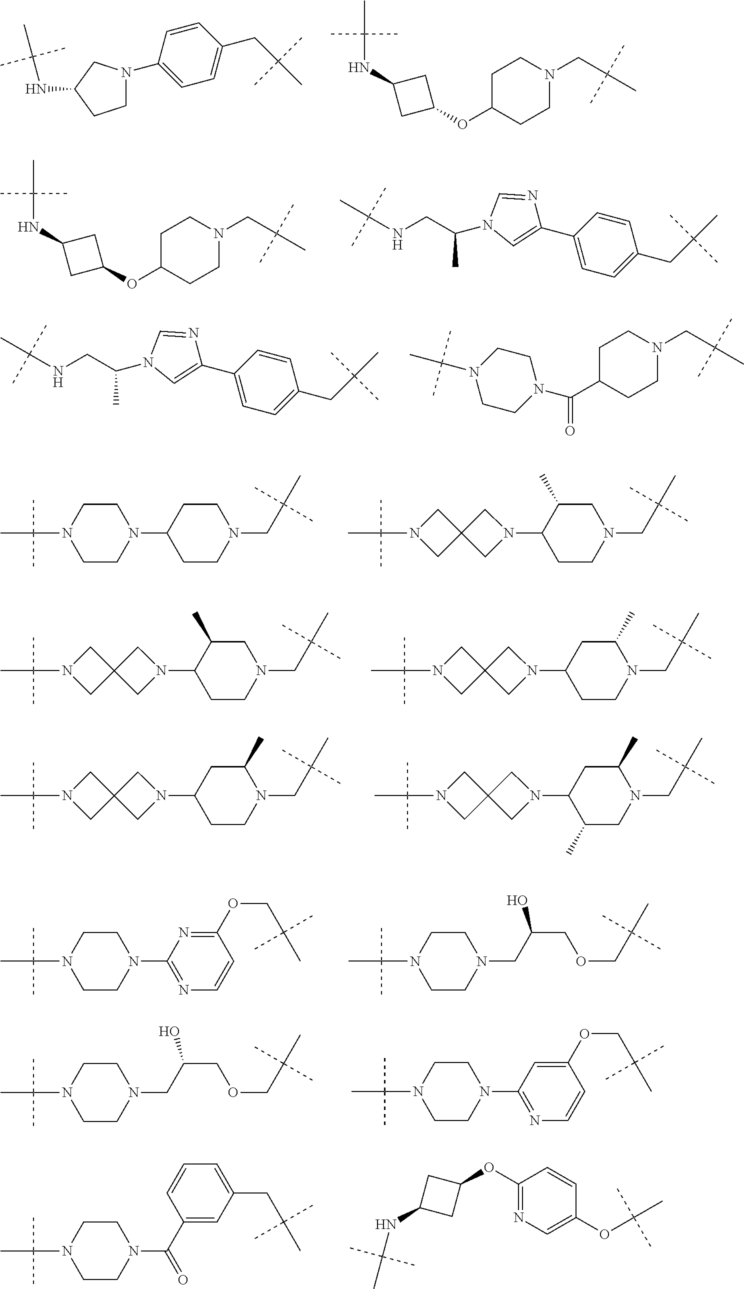

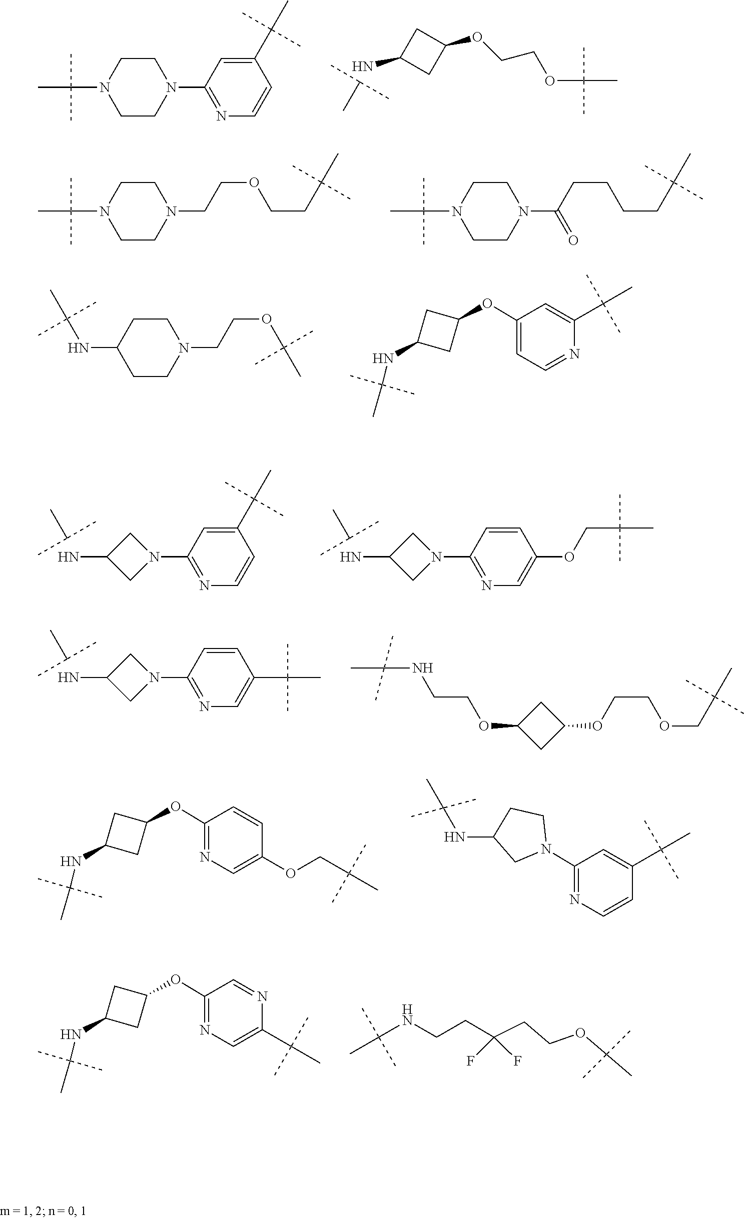

where n and m of the linker can be 0, 1, 2, 3, 4, 5, 6.

In additional embodiments, the linker (L) comprises a structure selected from, but not limited to the structure shown below, where a dashed line indicates the attachment point to the PTM or ULM moieties:

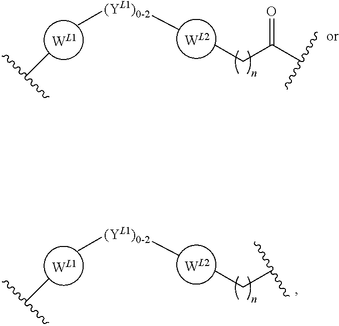

wherein:
W^{L1} and W^{L2} are each independently absent, a 4-8 membered ring with 0-4 heteroatoms, optionally substituted with R^{Q}, each R^{Q} is independently a H, halo, OH, CN, CF₃, C₁-C₆ alkyl (linear, branched, optionally substituted), C₁-C₆ alkoxy (linear, branched, optionally substituted), or 2 R^{Q} groups taken together with the atom they are attached to, form a 4-8 membered ring system containing 0-4 heteroatoms;
Y^{u} is each independently a bond, C₁-C₆ alkyl (linear, branched, optionally substituted) and optionally one or more C atoms are replaced with O; or C₁-C₆ alkoxy (linear, branched, optionally substituted);
n is 0-10; and
a dashed line indicates the attachment point to the PTM or ULM moieties.

In additional embodiments, the linker (L) comprises a structure selected from, but not limited to the structure shown below, where a dashed line indicates the attachment point to the PTM or ULM moieties:

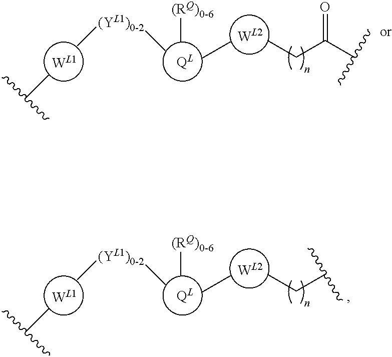

wherein:
W^{L1} and W^{L2} are each independently absent, aryl, heteroaryl, cyclic, heterocyclic, C₁₋₆ alkyl and optionally one or more C atoms are replaced with O, C₁₋₆ alkene and optionally one or more C atoms are replaced with O, C₁₋₆ alkyne and optionally one or more C atoms are replaced with O, bicyclic, biaryl, biheteroaryl, or biheterocyclic, each optionally substituted with R^{Q} each R^{Q} is independently a H, halo, OH, CN, CF₃, hydroxyl, nitro, C≡CH, C₂₋₆ alkenyl, C₂₋₆ alkynyl, C₁-C₆ alkyl (linear, branched, optionally substituted), C₁-C₆ alkoxy (linear, branched, optionally substituted), OC₁₋₃alkyl (optionally substituted by 1 or more —F), OH, NH₂, NR^{Y1}R^{Y2}, CN, or 2 R^{Q} groups taken together with the atom they are attached to, form a 4-8 membered ring system containing 0-4 heteroatoms;
Y^{L1} is each independently a bond, NR^{YL1}, O, S, NR^{YL2}, CR^{YL1}R^{YL2}, C═O, C═S, SO, SO₂, C₁-C₆ alkyl (linear, branched, optionally substituted) and optionally one or more C atoms are replaced with O; C₁-C₆ alkoxy (linear, branched, optionally substituted);
Q^{L} is a 3-6 membered alicyclic or aromatic ring with 0-4 heteroatoms, optionally bridged, optionally substituted with 0-6 R^{Q}, each R^{Q} is independently H, C₁₋₆ alkyl (linear, branched, optionally substituted by 1 or more halo, C₁₋₆ alkoxyl), or 2 R^{Q} groups taken together with the atom they are attached to, form a 3-8 membered ring system containing 0-2 heteroatoms);
R^{YL1}, R^{YL2} are each independently H, OH, C₁₋₆ alkyl (linear, branched, optionally substituted by 1 or more halo, C₁₋₆ alkoxyl), or R¹, R² together with the atom they are attached to, form a 3-8 membered ring system containing 0-2 heteroatoms);
n is 0-10; and
a dashed line indicates the attachment point to the PTM or ULM moieties.

In additional embodiments, the linker group is optionally substituted (poly)ethyleneglycol having between 1 and about 100 ethylene glycol units, between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units, or optionally substituted alkyl groups interdispersed with optionally substituted, O, N, S, P or Si atoms. In certain embodiments, the linker is substituted with an aryl, phenyl, benzyl, alkyl, alkylene, or heterocycle group. In certain embodiments, the linker may be asymmetric or symmetrical.

In any of the embodiments of the compounds described herein, the linker group may be any suitable moiety as described herein. In one embodiment, the linker is a substituted or unsubstituted polyethylene glycol group ranging in size from about 1 to about 12 ethylene glycol units, between 1 and about 10 ethylene glycol units, about 2 about 6 ethylene glycol units, between about 2 and 5 ethylene glycol units, between about 2 and 4 ethylene glycol units.

In another embodiment, the present disclosure is directed to a compound which comprises a PTM group as described above, which binds to a target protein or polypeptide (e.g., cMet or MET or p38), which is ubiquitinated by an ubiquitin ligase and is chemically linked directly to the ULM group or through a linker moiety L, or PTM is alternatively a ULM′ group which is also a ubiquitin ligase binding moiety, which may be the same or different than the ULM group as described above and is linked directly to the ULM group directly or through the linker moiety; and L is a linker moiety as described above which may be present or absent and which chemically (covalently) links ULM to PTM, or a pharmaceutically acceptable salt, enantiomer, stereoisomer, solvate or polymorph thereof.

In certain embodiments, the linker group L is a group comprising one or more covalently connected structural units independently selected from the group consisting of:

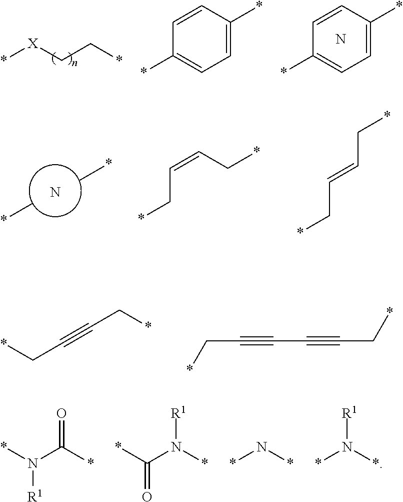

The X is selected from the group consisting of O, N, S, S(O) and SO₂; n is integer from 1 to 5; R^{L1} is hydrogen or alkyl,

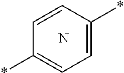

is a mono- or bicyclic aryl or heteroaryl optionally substituted with 1-3 substituents selected from alkyl, halogen, haloalkyl, hydroxy, alkoxy or cyano;

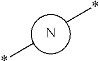

is a mono- or bicyclic cycloalkyl or a heterocycloalkyl optionally substituted with 1-3 substituents selected from alkyl, halogen, haloalkyl, hydroxy, alkoxy or cyano; and the phenyl ring fragment can be optionally substituted with 1, 2 or 3 substituents selected from the group consisting of alkyl, halogen, haloalkyl, hydroxy, alkoxy and cyano. In an embodiment, the linker group L comprises up to 10 covalently connected structural units, as described above.

Although the ULM group and PTM group may be covalently linked to the linker group through any group which is appropriate and stable to the chemistry of the linker, in preferred aspects of the present disclosure, the linker is independently covalently bonded to the ULM group and the PTM group preferably through an amide, ester, thioester, keto group, carbamate (urethane), carbon or ether, each of which groups may be inserted anywhere on the ULM group and PTM group to provide maximum binding of the ULM group on the ubiquitin ligase and the PTM group on the target protein to be degraded. (It is noted that in certain aspects where the PTM group is a ULM group, the target protein for degradation may be the ubiquitin ligase itself). In certain preferred aspects, the linker may be linked to an optionally substituted alkyl, alkylene, alkene or alkyne group, an aryl group or a heterocyclic group on the ULM and/or PTM groups.

### Exemplary PTMs

In preferred aspects of the disclosure, the PTM group is a group, which binds to target proteins, e.g. c-Met. Targets of the PTM group are numerous in kind and are selected from proteins that are expressed in a cell such that at least a portion of the sequences is found in the cell and may bind to a PTM group. The term “protein” includes oligopeptides and polypeptide sequences of sufficient length that they can bind to a PTM group according to the present disclosure. Any protein in a eukaryotic system or a microbial system, including a virus, bacteria or fungus, as otherwise described herein, are targets for ubiquitination mediated by the compounds according to the present disclosure. Preferably, the target protein is a eukaryotic protein.

PTM groups according to the present disclosure include, for example, any moiety which binds to a protein specifically (binds to a target protein) and includes the following non-limiting examples of small molecule target protein moieties: p38 inhibitors, cMet inhibitors, Hsp90 inhibitors, kinase inhibitors, HDM2 & MDM2 inhibitors, compounds targeting Human BET Bromodomain-containing proteins, HDAC inhibitors, human lysine methyltransferase inhibitors, angiogenesis inhibitors, nuclear hormone receptor compounds, immunosuppressive compounds, and compounds targeting the aryl hydrocarbon receptor (AHR), among numerous others. The compositions described below exemplify some of the members of small molecule target protein binding moieties. Such small molecule target protein binding moieties also include pharmaceutically acceptable salts, enantiomers, solvates and polymorphs of these compositions, as well as other small molecules that may target a protein of interest. These binding moieties are linked to the ubiquitin ligase binding moiety preferably through a linker in order to present a target protein (to which the protein target moiety is bound) in proximity to the ubiquitin ligase for ubiquitination and degradation.

Any protein, which can bind to a protein target moiety or PTM group and acted on or degraded by an ubiquitin ligase is a target protein according to the present disclosure. In general, target proteins may include, for example, structural proteins, receptors, enzymes, cell surface proteins, proteins pertinent to the integrated function of a cell, including proteins involved in catalytic activity, aromatase activity, motor activity, helicase activity, metabolic processes (anabolism and catabolism), antioxidant activity, proteolysis, biosynthesis, proteins with kinase activity, oxidoreductase activity, transferase activity, hydrolase activity, lyase activity, isomerase activity, ligase activity, enzyme regulator activity, signal transducer activity, structural molecule activity, binding activity (protein, lipid carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport (including protein transporter activity, nuclear transport, ion transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity, translation regulator activity. Proteins of interest can include proteins from eukaryotes and prokaryotes including humans as targets for drug therapy, other animals, including domesticated animals, microbials for the determination of targets for antibiotics and other antimicrobials and plants, and even viruses, among numerous others.

In certain embodiments, the protein targeting moiety or PTM is selected from:

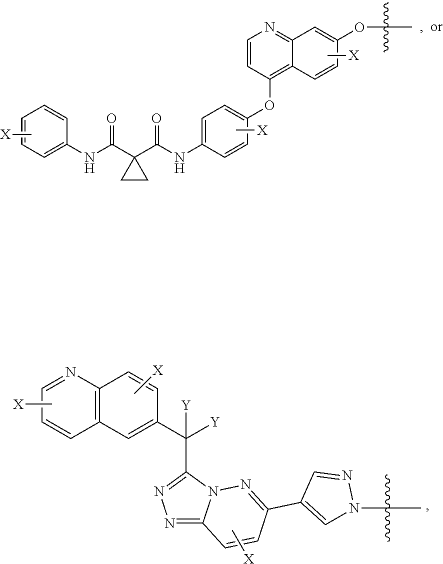

wherein each X is independently Cl, F, Br, H, CN, Me, OMe, or OCF₃; and each Y is independently F or H.

The present disclosure may be used to treat a number of disease states and/or conditions, including any disease state and/or condition in which proteins are dysregulated and where a patient would benefit from the degradation and/or inhibition of proteins.

In an additional aspect, the description provides therapeutic compositions comprising an effective amount of a compound as described herein or salt form thereof, and a pharmaceutically acceptable carrier, additive or excipient, and optionally an additional bioactive agent. The therapeutic compositions modulate protein degradation in a patient or subject, for example, an animal such as a human, and can be used for treating or ameliorating disease states or conditions which are modulated through the degraded protein. In certain embodiments, the therapeutic compositions as described herein may be used to effectuate the degradation of proteins of interest for the treatment or amelioration of a disease, e.g., cancer. In certain additional embodiments, the disease is at least one cancer selected from the group consisting of gastric, non-small cell lung cancer, advanced hepatocellular carcinoma (HCC), papillary renal cell cancer (RCC), or a combination thereof.

In alternative aspects, the present disclosure relates to a method for treating a disease state or ameliorating the symptoms of a disease or condition in a subject in need thereof by degrading a protein or polypeptide through which a disease state or condition is modulated comprising administering to said patient or subject an effective amount, e.g., a therapeutically effective amount, of at least one compound as described hereinabove, optionally in combination with a pharmaceutically acceptable carrier, additive or excipient, and optionally an additional bioactive agent, wherein the composition is effective for treating or ameliorating the disease or disorder or symptom thereof in the subject. The method according to the present disclosure may be used to treat a large number of disease states or conditions including cancer, by virtue of the administration of effective amounts of at least one compound described herein. The disease state or condition may be a disease caused by a microbial agent or other exogenous agent such as a virus, bacteria, fungus, protozoa or other microbe or may be a disease state, which is caused by overexpression of a protein, such as cMet and/or p38, which leads to a disease state and/or condition.

In another aspect, the description provides methods for identifying the effects of the degradation of proteins of interest in a biological system using compounds according to the present disclosure.

The term “target protein” is used to describe a protein or polypeptide, which is a target for binding to a compound according to the present disclosure and degradation by ubiquitin ligase hereunder. Such small molecule target protein binding moieties also include pharmaceutically acceptable salts, enantiomers, solvates and polymorphs of these compositions, as well as other small molecules that may target a protein of interest, such as MET. These binding moieties are linked to at least one ULM group (e.g. VLM, CLM, ILM, and/or MLM) through at least one linker group L.

Target proteins, which may be bound to the protein target moiety and degraded by the ligase to which the ubiquitin ligase binding moiety is bound, include any protein or peptide, including fragments thereof, analogues thereof, and/or homologues thereof. Target proteins include proteins and peptides having any biological function or activity including structural, regulatory, hormonal, enzymatic, genetic, immunological, contractile, storage, transportation, and signal transduction. More specifically, a number of drug targets for human therapeutics represent protein targets to which protein target moiety may be bound and incorporated into compounds according to the present disclosure. These include proteins which may be used to restore function in numerous polygenic diseases, including for example c-Met, B7.1 and B7, TINFR1m, TNFR2, NADPH oxidase, BclIBax and other partners in the apotosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P-glycoprotein (and MRP), tyrosine kinases, CD23, CD124, tyrosine kinase p56 lck, CD4, CD5, IL-2 receptor, IL-1 receptor, TNF-alphaR, ICAM1, Cat+ channels, VCAM, VLA-4 integrin, selectins, CD40/CD40L, newokinins and receptors, inosine monophosphate dehydrogenase, p38 MAP Kinase, RaslRaflMEWERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, estrogen receptors, androgen receptors, adenosine receptors, adenosine kinase and AMP deaminase, purinergic receptors (P2Y1, P2Y2, P2Y4, P2Y6, P2X1-7), farnesyltransferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase. Additional protein targets include, for example, ecdysone 20-monooxygenase, ion channel of the GABA gated chloride channel, acetylcholinesterase, voltage-sensitive sodium channel protein, calcium release channel, and chloride channels. Still further target proteins include Acetyl-CoA carboxylase, adenylosuccinate synthetase, protoporphyrinogen oxidase, and enolpyruvylshikimate-phosphate synthase.

These various protein targets may be used in screens that identify compound moieties which bind to the protein and by incorporation of the moiety into compounds according to the present disclosure, the level of activity of the protein may be altered for therapeutic end result.

The term “protein target moiety” or PTM is used to describe a small molecule which binds to a target protein or other protein or polypeptide of interest and places/presents that protein or polypeptide in proximity to an ubiquitin ligase such that degradation of the protein or polypeptide by ubiquitin ligase may occur. Non-limiting examples of small molecule target protein binding moieties include c-Met inhibitors, Hsp90 inhibitors, kinase inhibitors, MDM2 inhibitors, compounds targeting Human BET Bromodomain-containing proteins, HDAC inhibitors, human lysine methyltransferase inhibitors, angiogenesis inhibitors, immunosuppressive compounds, and compounds targeting the aryl hydrocarbon receptor (AHR), among numerous others. The compositions described below exemplify some of the members of the small molecule target proteins.

The compositions described herein exemplify some of the members of these types of small molecule target protein binding moieties. Such small molecule target protein binding moieties also include pharmaceutically acceptable salts, enantiomers, solvates and polymorphs of these compositions, as well as other small molecules that may target a protein of interest. References which are cited herein below are incorporated by reference herein in their entirety.

In certain embodiments, the bifunctional compound of the present disclosure has one of the following chemical structures:

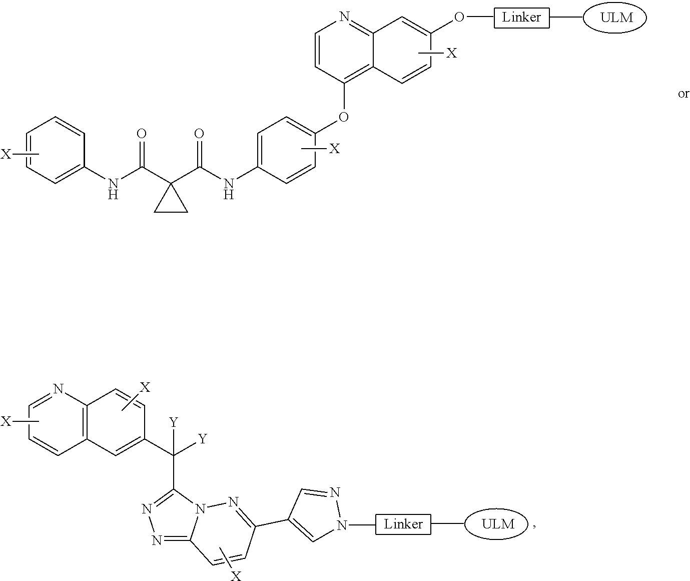

wherein:

each X is independently Cl, F, Br, H, CN, Me, OMe, OCF3; and

each Y is independently F or H.

### Therapeutic Compositions

Pharmaceutical compositions comprising combinations of an effective amount of at least one bifunctional compound as described herein, and one or more of the compounds otherwise described herein, all in effective amounts, in combination with a pharmaceutically effective amount of a carrier, additive or excipient, represents a further aspect of the present disclosure.

The present disclosure includes, where applicable, the compositions comprising the pharmaceutically acceptable salts, in particular, acid or base addition salts of compounds as described herein. The acids which are used to prepare the pharmaceutically acceptable acid addition salts of the aforementioned base compounds useful according to this aspect are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among numerous others.

Pharmaceutically acceptable base addition salts may also be used to produce pharmaceutically acceptable salt forms of the compounds or derivatives according to the present disclosure. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (eg., potassium and sodium) and alkaline earth metal cations (eg, calcium, zinc and magnesium), ammonium or water-soluble amine addition salts such as N-methylglucamine-(meglumine), and the lower alkanolammonium and other base salts of pharmaceutically acceptable organic amines, among others.

The compounds as described herein may, in accordance with the disclosure, be administered in single or divided doses by the oral, parenteral or topical routes. Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal, sublingual and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Administration of compounds according to the present disclosure as sprays, mists, or aerosols for intra-nasal, intra-tracheal or pulmonary administration may also be used. The present disclosure therefore also is directed to pharmaceutical compositions comprising an effective amount of compound as described herein, optionally in combination with a pharmaceutically acceptable carrier, additive or excipient. Compounds according to the present disclosure may be administered in immediate release, intermediate release or sustained or controlled release forms. Sustained or controlled release forms are preferably administered orally, but also in suppository and transdermal or other topical forms. Intramuscular injections in liposomal form may also be used to control or sustain the release of compound at an injection site.

The compositions as described herein may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions as described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions as described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions as described herein may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions as described herein may be administered in the form of suppositories for rectal administration. These can be prepared by mixing the agent with a suitable non-irritating excipient, which is solid at room temperature but liquid at rectal temperature and therefore will melt in the rectum to release the drug. Such materials include cocoa butter, beeswax and polyethylene glycols.

The pharmaceutical compositions as described herein may also be administered topically. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. In certain preferred aspects of the disclosure, the compounds may be coated onto a stent which is to be surgically implanted into a patient in order to inhibit or reduce the likelihood of occlusion occurring in the stent in the patient.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions as described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition as described herein that may be combined with the carrier materials to produce a single dosage form will vary depending upon the host and disease treated, the particular mode of administration. Preferably, the compositions should be formulated to contain between about 0.05 milligram to about 750 milligrams or more, more preferably about 1 milligram to about 600 milligrams, and even more preferably about 10 milligrams to about 500 milligrams of active ingredient, alone or in combination with at least one other compound according to the present disclosure.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

A patient or subject in need of therapy using compounds according to the methods described herein can be treated by administering to the patient (subject) an effective amount of the compound according to the present disclosure including pharmaceutically acceptable salts, solvates or polymorphs, thereof optionally in a pharmaceutically acceptable carrier or diluent, either alone, or in combination with other known therapeutic agents as otherwise identified herein.

These compounds can be administered by any appropriate route, for example, orally, parenterally, intravenously, intradermally, subcutaneously, or topically, including transdermally, in liquid, cream, gel, or solid form, or by aerosol form.

The active compound is included in the pharmaceutically acceptable carrier or diluent in an amount sufficient to deliver to a patient a therapeutically effective amount for the desired indication, without causing serious toxic effects in the patient treated. A preferred dose of the active compound for all of the herein-mentioned conditions is in the range from about 10 ng/kg to 300 mg/kg, preferably 0.1 to 100 mg/kg per day, more generally 0.5 to about 25 mg per kilogram body weight of the recipient/patient per day. A typical topical dosage will range from 0.01-5% wt/wt in a suitable carrier.

The compound is conveniently administered in any suitable unit dosage form, including but not limited to one containing less than 1 mg, 1 mg to 3000 mg, preferably 5 to 500 mg of active ingredient per unit dosage form. An oral dosage of about 25-250 mg is often convenient.

The active ingredient is preferably administered to achieve peak plasma concentrations of the active compound of about 0.00001-30 mM, preferably about 0.1-30 μM. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as anti-cancer agents. In certain preferred aspects of the disclosure, one or more compounds according to the present disclosure are coadministered with another bioactive agent, such as an anti-cancer agent or a would-healing agent, including an antibiotic, as otherwise described herein.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension.

### Therapeutic Methods

In an additional aspect, the description provides therapeutic compositions comprising an effective amount of a compound as described herein or salt form thereof, and a pharmaceutically acceptable carrier. The therapeutic compositions modulate protein degradation in a patient or subject, for example, an animal such as a human, and can be used for treating or ameliorating disease states or conditions which are modulated through the degraded protein.

The terms “treat”, “treating”, and “treatment”, etc., as used herein, refer to any action providing a benefit to a patient for which the present compounds may be administered, including the treatment of any disease state or condition which is modulated through the protein to which the present compounds bind. Disease states or conditions, including cancer, which may be treated using compounds according to the present disclosure are set forth hereinabove.

The description provides therapeutic compositions as described herein for effectuating the degradation of proteins of interest for the treatment or amelioration of a disease, e.g., cancer. In certain additional embodiments, the disease is multiple myeloma. As such, in another aspect, the description provides a method of ubiquitinating/degrading a target protein in a cell. In certain embodiments, the method comprises administering a bifunctional compound as described herein comprising, e.g., a ULM and a PTM, preferably linked through a linker moiety, as otherwise described herein, wherein the ULM is coupled to the PTM and wherein the ULM recognizes a ubiquitin pathway protein (e.g., an ubiquitin ligase, such as an E3 ubiquitin ligase including cereblon, VHL, IAP, and/or MDM2) and the PTM recognizes the target protein such that degradation of the target protein will occur when the target protein is placed in proximity to the ubiquitin ligase, thus resulting in degradation/inhibition of the effects of the target protein and the control of protein levels. The control of protein levels afforded by the present disclosure provides treatment of a disease state or condition, which is modulated through the target protein by lowering the level of that protein in the cell, e.g., cell of a patient. In certain embodiments, the method comprises administering an effective amount of a compound as described herein, optionally including a pharmaceutically acceptable excipient, carrier, adjuvant, another bioactive agent or combination thereof.

In additional embodiments, the description provides methods for treating or ameliorating a disease, disorder or symptom thereof in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for treating or ameliorating the disease or disorder or symptom thereof in the subject.

In another aspect, the description provides methods for identifying the effects of the degradation of proteins of interest in a biological system using compounds according to the present disclosure.

In another embodiment, the present disclosure is directed to a method of treating a human patient in need for a disease state or condition modulated through a protein where the degradation of that protein will produce a therapeutic effect in the patient, the method comprising administering to a patient in need an effective amount of a compound according to the present disclosure, optionally in combination with another bioactive agent. The disease state or condition may be a disease caused by a microbial agent or other exogenous agent such as a virus, bacteria, fungus, protozoa or other microbe or may be a disease state, which is caused by overexpression of a protein, which leads to a disease state and/or condition

The term “disease state or condition” is used to describe any disease state or condition wherein protein dysregulation (i.e., the amount of protein expressed in a patient is elevated) occurs and where degradation of one or more proteins in a patient may provide beneficial therapy or relief of symptoms to a patient in need thereof. In certain instances, the disease state or condition may be cured.

Disease states or conditions which may be treated using compounds according to the present disclosure include, for example, asthma, autoimmune diseases such as multiple sclerosis, various cancers, ciliopathies, cleft palate, diabetes, heart disease, hypertension, inflammatory bowel disease, mental retardation, mood disorder, obesity, refractive error, infertility, Angelman syndrome, Canavan disease, Coeliac disease, Charcot-Marie-Tooth disease, Cystic fibrosis, Duchenne muscular dystrophy, Haemochromatosis, Haemophilia, Klinefelter's syndrome, Neurofibromatosis, Phenylketonuria, Polycystic kidney disease, (PKD1) or 4 (PKD2) Prader-Willi syndrome, Sickle-cell disease, Tay-Sachs disease, Turner syndrome.

The term “neoplasia” or “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Exemplary cancers which may be treated by the present compounds either alone or in combination with at least one additional anti-cancer agent include gastric, non-small cell lung cancer, squamous-cell carcinoma, basal cell carcinoma, adenocarcinoma, hepatocellular carcinomas, advanced hepatocellular carcinoma, renal cell carcinomas, and papillary renal cell carcinoma, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present disclosure include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

The term “bioactive agent” is used to describe an agent, other than a compound according to the present disclosure, which is used in combination with the present compounds as an agent with biological activity to assist in effecting an intended therapy, inhibition and/or prevention/prophylaxis for which the present compounds are used. Preferred bioactive agents for use herein include those agents which have pharmacological activity similar to that for which the present compounds are used or administered and include for example, anti-cancer agents, antiviral agents, especially including anti-HIV agents and anti-HCV agents, antimicrobial agents, antifungal agents, etc.

The term “additional anti-cancer agent” is used to describe an anti-cancer agent, which may be combined with compounds according to the present disclosure to treat cancer. These agents include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitor, an AKT inhibitor, an mTORC1/2 inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

The term “anti-HIV agent” or “additional anti-HIV agent” includes, for example, nucleoside reverse transcriptase inhibitors (NRTI), other non-nucloeoside reverse transcriptase inhibitors (i.e., those which are not representative of the present disclosure), protease inhibitors, fusion inhibitors, among others, exemplary compounds of which may include, for example, 3TC (Lamivudine), AZT (Zidovudine), (−)-FTC, ddI (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof, including anti-HIV compounds presently in clinical trials or in development.

Other anti-HIV agents which may be used in coadministration with compounds according to the present disclosure include, for example, other NNRTI's (i.e., other than the NNRTI's according to the present disclosure) may be selected from the group consisting of nevirapine (BI-R6-587), delavirdine (U-90152S/T), efavirenz (DMP-266), UC-781 (N-[4-chloro-3-(3-methyl-2-butenyloxy)phenyl]-2methyl3-furancarbothiamide), etravirine (TMC 125), Trovirdine (Ly300046.HCl), MKC-442 (emivirine, coactinon), HI-236, HI-240, HI-280, HI-281, rilpivirine (TMC-278), MSC-127, HBY 097, DMP266, Baicalin (TJN-151) ADAM-II (Methyl 3′,3′-dichloro-4′,4″-dimethoxy-5′,5″-bis(methoxycarbonyl)-6,6-diphenylhexenoate), Methyl 3-Bromo-5-(1-5-bromo-4-methoxy-3-(methoxycarbonyl)phenyl)hept-1-enyl)-2-methoxybenzoate (Alkenyldiarylmethane analog, Adam analog), (5-chloro-3-(phenylsulfinyl)-2′-indolecarboxamide), AAP-BHAP (U-104489 or PNU-104489), Capravirine (AG-1549, S-1153), atevirdine (U-87201E), aurin tricarboxylic acid (SD-095345), 1-[(6-cyano-2-indolyl)carbonyl]-4-[3-(isopropylamino)-2-pyridinyl]piperazine, 1-[5-[[N-(methyl)methylsulfonylamino]-2-indolylcarbonyl-4-[3-(isopropylamino)-2-pyridinyl]piperazine, 1-[3-(Ethylamino)-2-[pyridinyl]-4-[(5-hydroxy-2-indolyl)carbonyl]piperazine, 1-[(6-Formyl-2-indolyl)carbonyl]-4-[3-(isopropylamino)-2-pyridinyl]piperazine, 1-[[5-(Methylsulfonyloxy)-2-indoyly)carbonyl]-4-[3-(isopropylamino)-2-pyridinyl]piperazine, U88204E, Bis(2-nitrophenyl)sulfone (NSC 633001), Calanolide A (NSC675451), Calanolide B, 6-Benzyl-5-methyl-2-(cyclohexyloxy)pyrimidin-4-one (DABO-546), DPC 961, E-EBU, E-EBU-dm, E-EPSeU, E-EPU, Foscarnet (Foscavir), HEPT (1-[(2-Hydroxyethoxy)methyl]-6-(phenylthio)thymine), HEPT-M (1-[(2-Hydroxyethoxy)methyl]-6-(3-methylphenyl)thio)thymine), HEPT-S (1-[(2-Hydroxyethoxy)methyl]-6-(phenylthio)-2-thiothymine), Inophyllum P, L-737,126, Michellamine A (NSC650898), Michellamine B (NSC649324), Michellamine F, 6-(3,5-Dimethylbenzyl)-1-[(2-hydroxyethoxy)methyl]-5-isopropyluracil, 6-(3,5-Dimethylbenzyl)-1-(ethyoxymethyl)-5-isopropyluracil, NPPS, E-BPTU (NSC 648400), Oltipraz (4-Methyl-5-(pyrazinyl)-3H-1,2-dithiole-3-thione), N-{2-(2-Chloro-6-fluorophenethyl]-N′-(2-thiazolyl)thiourea (PETT Cl, F derivative), N-{2-(2,6-Difluorophenethyl]-N′-[2-(5-bromopyridyl)]thiourea {PETT derivative), N-{2-(2,6-Difluorophenethyl]-N′-[2-(5-methylpyridyl]thiourea {PETT Pyridyl derivative), N-[2-(3-Fluorofuranyl)ethyl]-N′-[2-(5-chloropyridyl)]thiourea, N-[2-(2-Fluoro-6-ethoxyphenethyl)]-N′-[2-(5-bromopyridyl)]thiourea, N-(2-Phenethyl)-N′-(2-thiazolyl)thiourea (LY-73497), L-697,639, L-697,593, L-697,661, 3-[2-(4,7-Difluorobenzoxazol-2-yl)ethyl}-5-ethyl-6-methyl(pypridin-2(1H)-thione (2-Pyridinone Derivative), 3-[[(2-Methoxy-5,6-dimethyl-3-pyridyl)methyl]amine]-5-ethyl-6-methyl(pypridin-2(1H)-thione, R82150, R82913, R87232, R88703, R89439 (Loviride), R90385, S-2720, Suramin Sodium, TBZ (Thiazolobenzimidazole, NSC 625487), Thiazoloisoindol-5-one, (+)(R)-9b-(3,5-Dimethylphenyl-2,3-dihydrothiazolo[2,3-a]isoindol-5(9bH)-one, Tivirapine (R86183), UC-38 and UC-84, among others.

The term “pharmaceutically acceptable salt” is used throughout the specification to describe, where applicable, a salt form of one or more of the compounds described herein which are presented to increase the solubility of the compound in the gastric juices of the patient's gastrointestinal tract in order to promote dissolution and the bioavailability of the compounds. Pharmaceutically acceptable salts include those derived from pharmaceutically acceptable inorganic or organic bases and acids, where applicable. Suitable salts include those derived from alkali metals such as potassium and sodium, alkaline earth metals such as calcium, magnesium and ammonium salts, among numerous other acids and bases well known in the pharmaceutical art. Sodium and potassium salts are particularly preferred as neutralization salts of the phosphates according to the present disclosure.

The term “pharmaceutically acceptable derivative” is used throughout the specification to describe any pharmaceutically acceptable prodrug form (such as an ester, amide other prodrug group), which, upon administration to a patient, provides directly or indirectly the present compound or an active metabolite of the present compound.

### General Synthetic Approach

The synthetic realization and optimization of the bifunctional molecules as described herein may be approached in a step-wise or modular fashion. For example, identification of compounds that bind to the target molecules can involve high or medium throughput screening campaigns if no suitable ligands are immediately available. It is not unusual for initial ligands to require iterative design and optimization cycles to improve suboptimal aspects as identified by data from suitable in vitro and pharmacological and/or ADMET assays. Part of the optimization/SAR campaign would be to probe positions of the ligand that are tolerant of substitution and that might be suitable places on which to attach the linker chemistry previously referred to herein. Where crystallographic or NMR structural data are available, these can be used to focus such a synthetic effort.

In a very analogous way one can identify and optimize ligands for an E3 Ligase, i.e. ULMs/ILMs/VLMs/CLMs/ILMs.

With PTMs and ULMs (e.g. ILMs, VLMs, CLMs, and/or ILMs) in hand, one skilled in the art can use known synthetic methods for their combination with or without a linker moiety. Linker moieties can be synthesized with a range of compositions, lengths and flexibility and functionalized such that the PTM and ULM groups can be attached sequentially to distal ends of the linker. Thus a library of bifunctional molecules can be realized and profiled in in vitro and in vivo pharmacological and ADMET/PK studies. As with the PTM and ULM groups, the final bifunctional molecules can be subject to iterative design and optimization cycles in order to identify molecules with desirable properties.

In some instances, protecting group strategies and/or functional group interconversions (FGIs) may be required to facilitate the preparation of the desired materials. Such chemical processes are well known to the synthetic organic chemist and many of these may be found in texts such as “Greene's Protective Groups in Organic Synthesis” Peter G. M. Wuts and Theodora W. Greene (Wiley), and “Organic Synthesis: The Disconnection Approach” Stuart Warren and Paul Wyatt (Wiley).

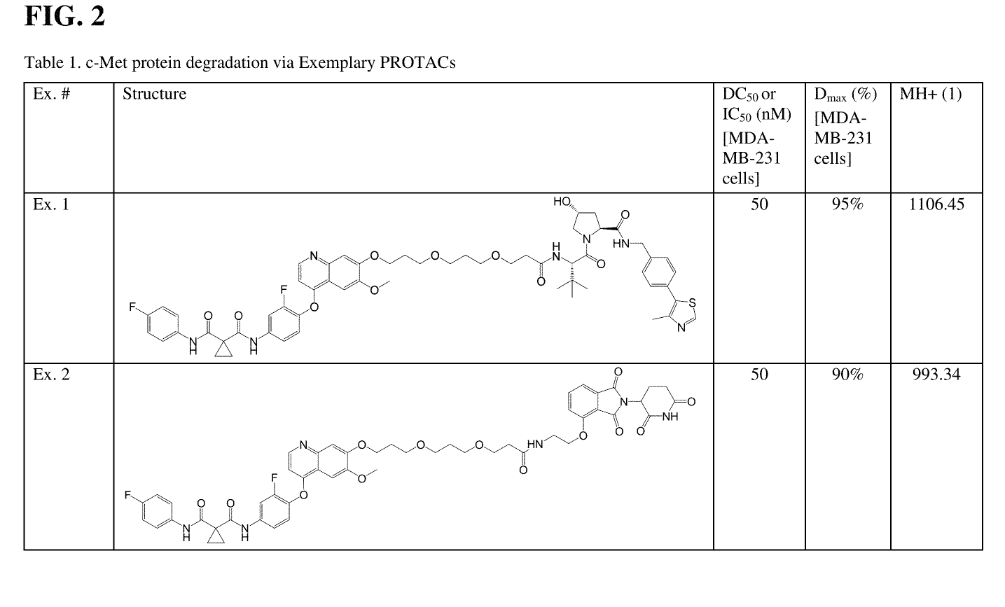

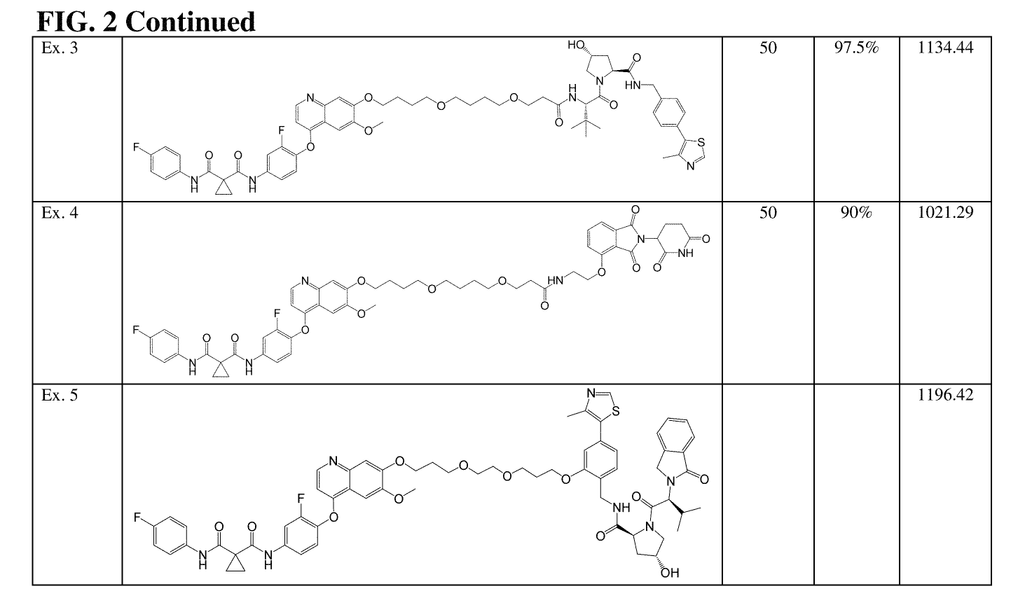

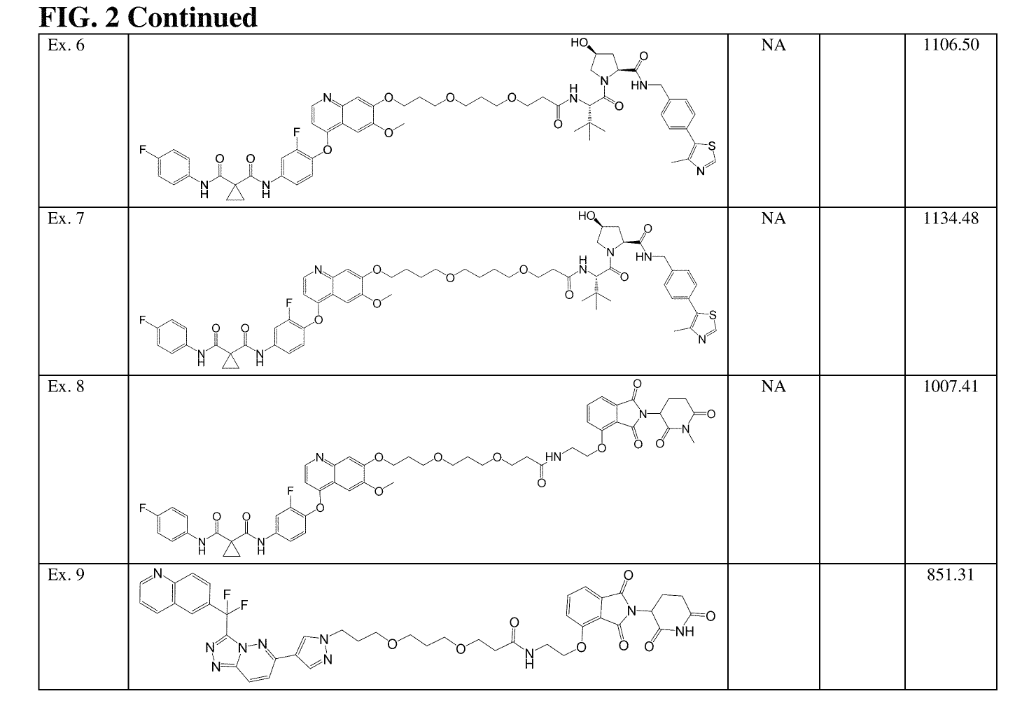

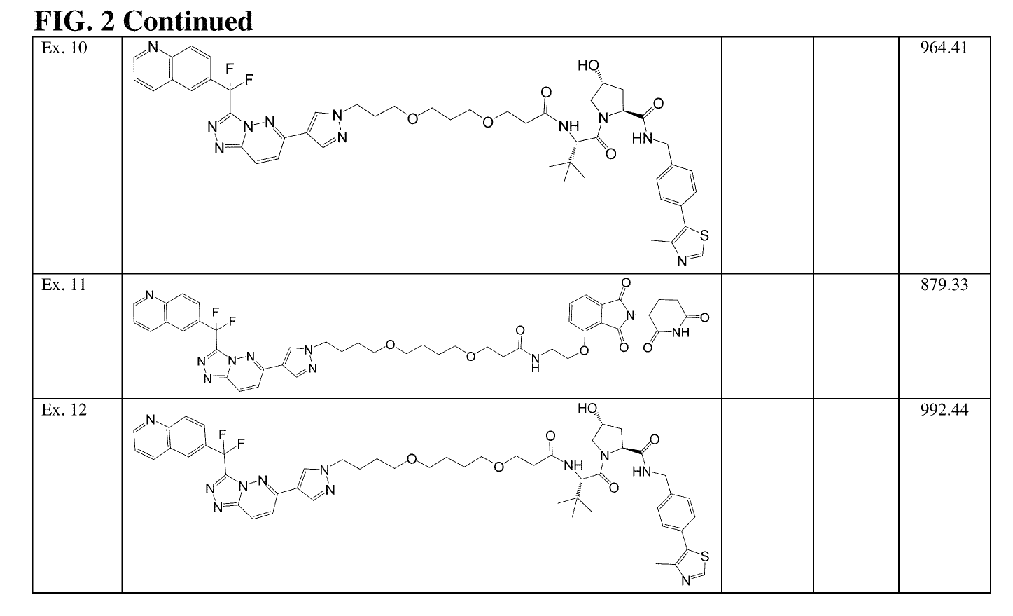

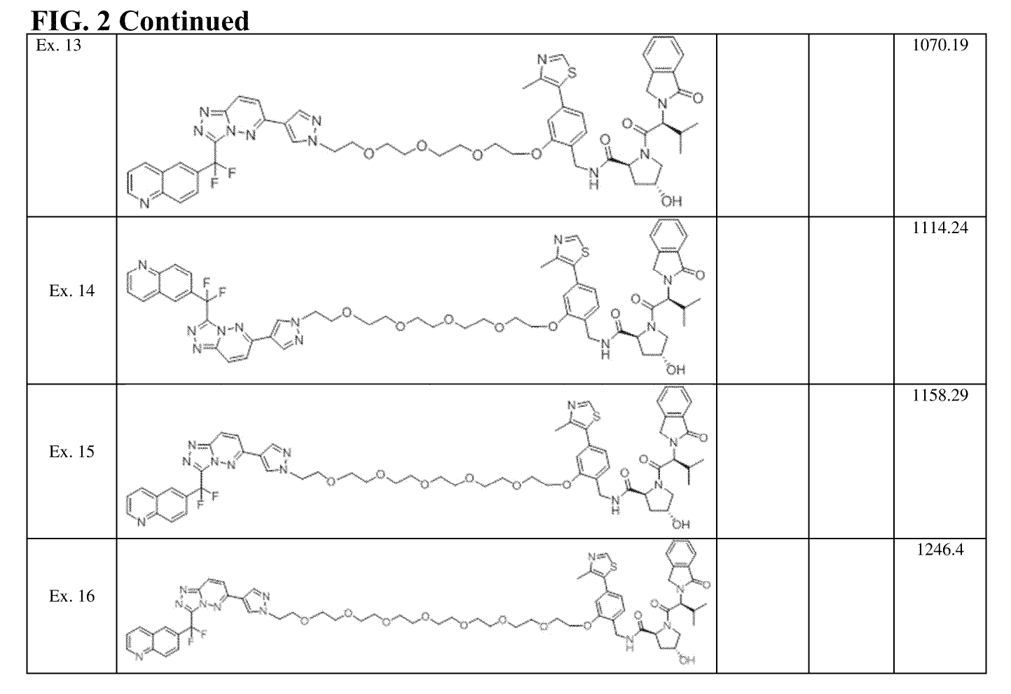

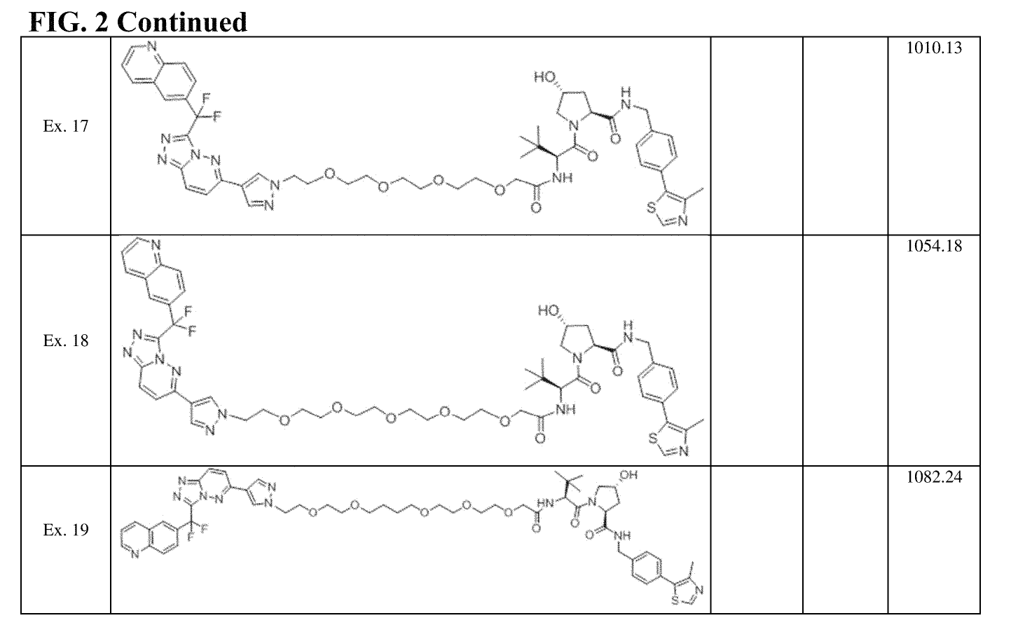

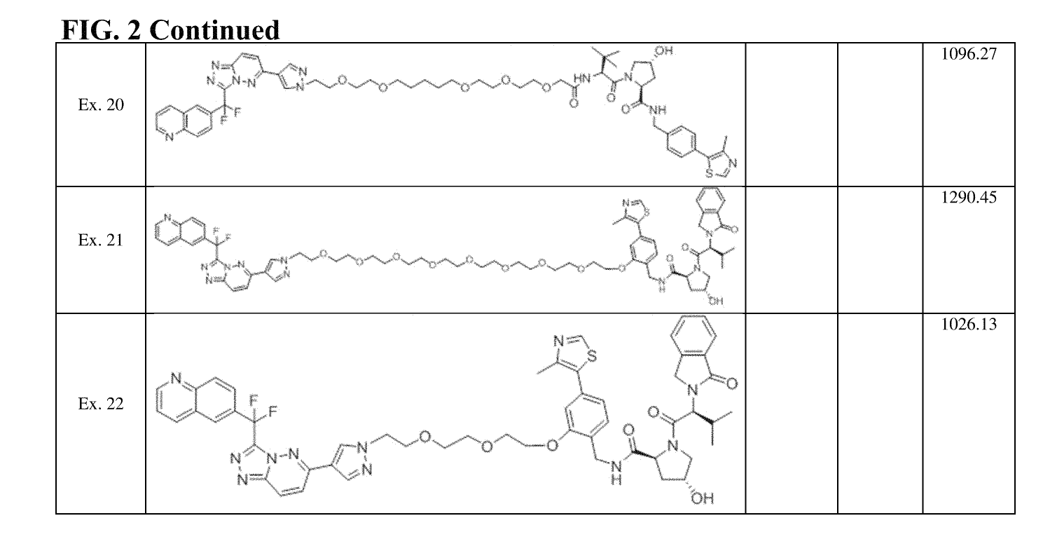

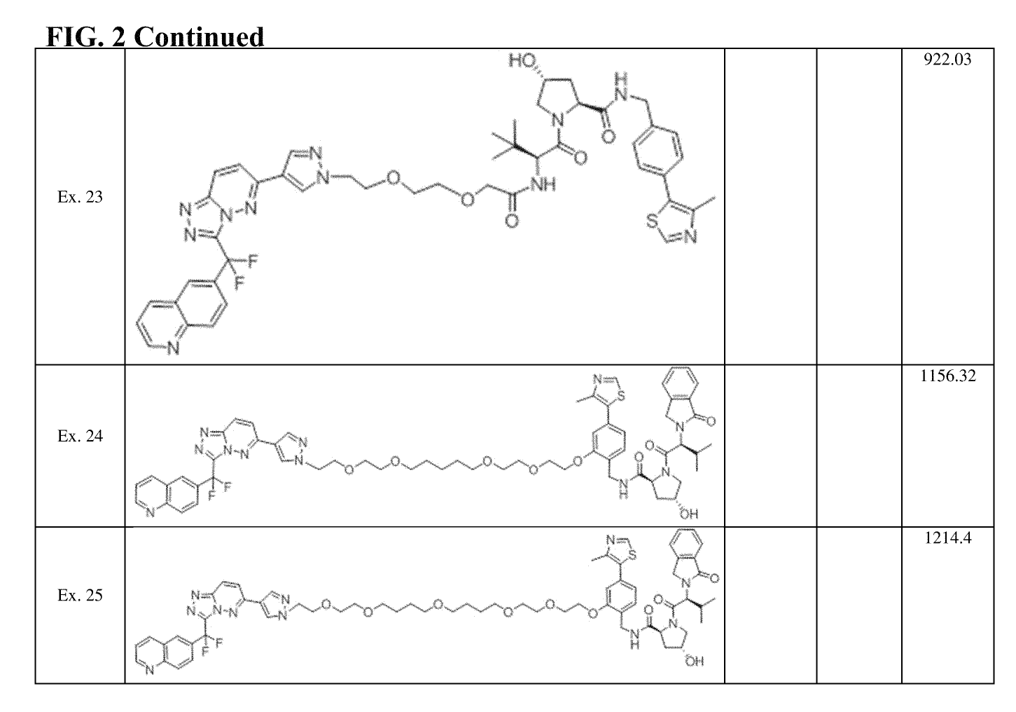

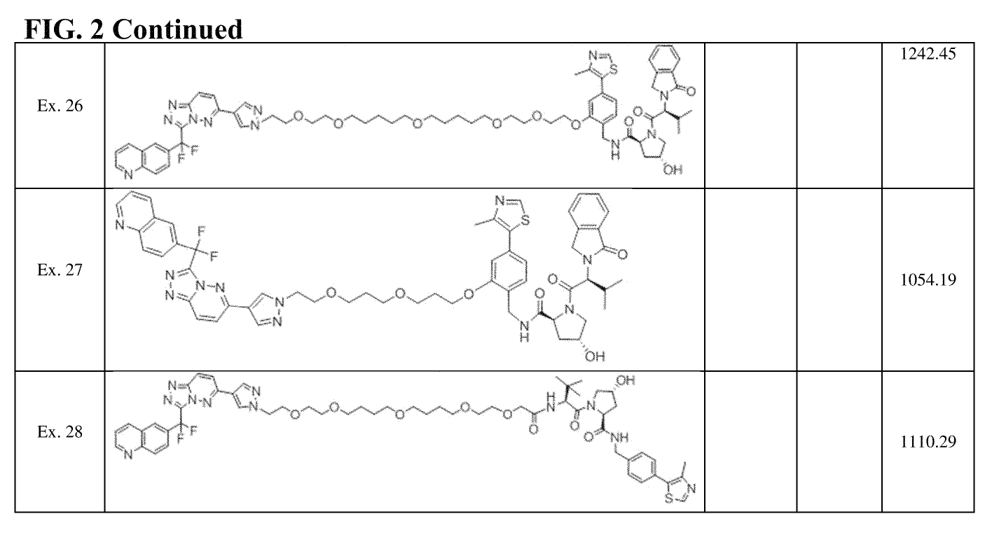

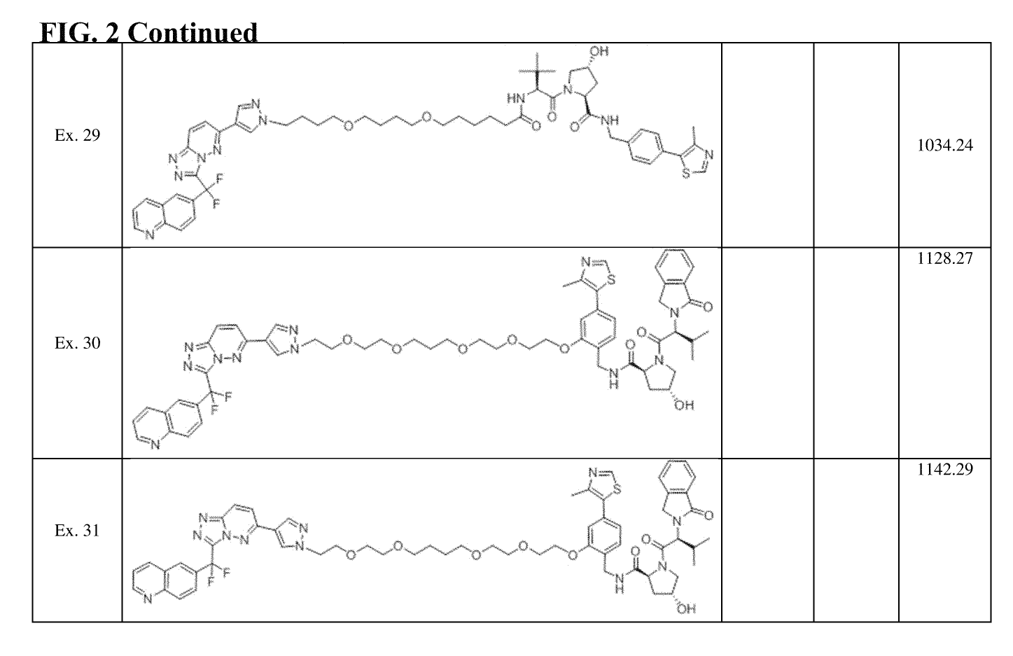

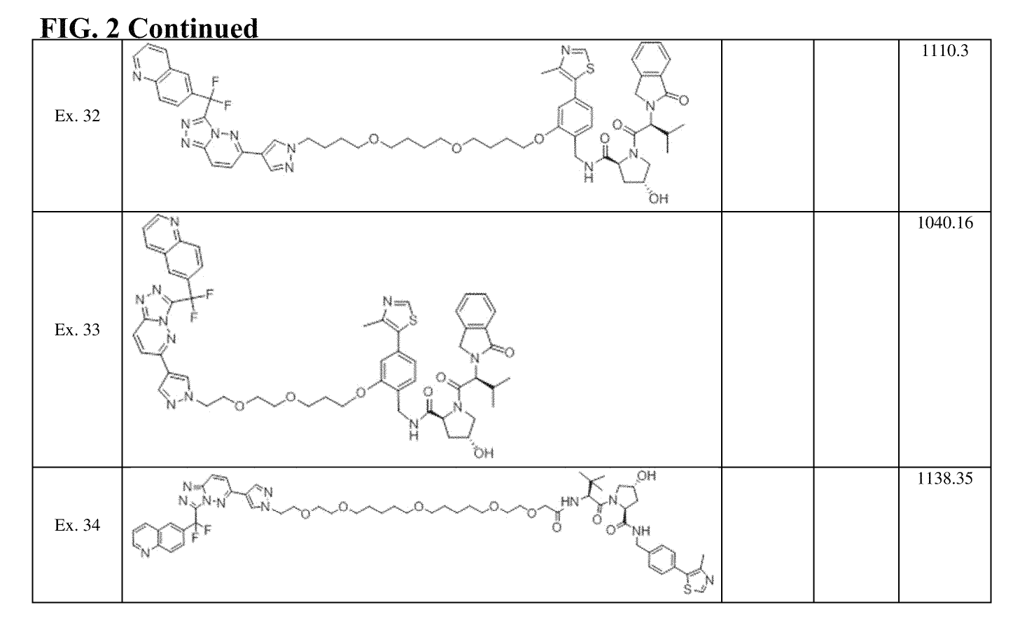

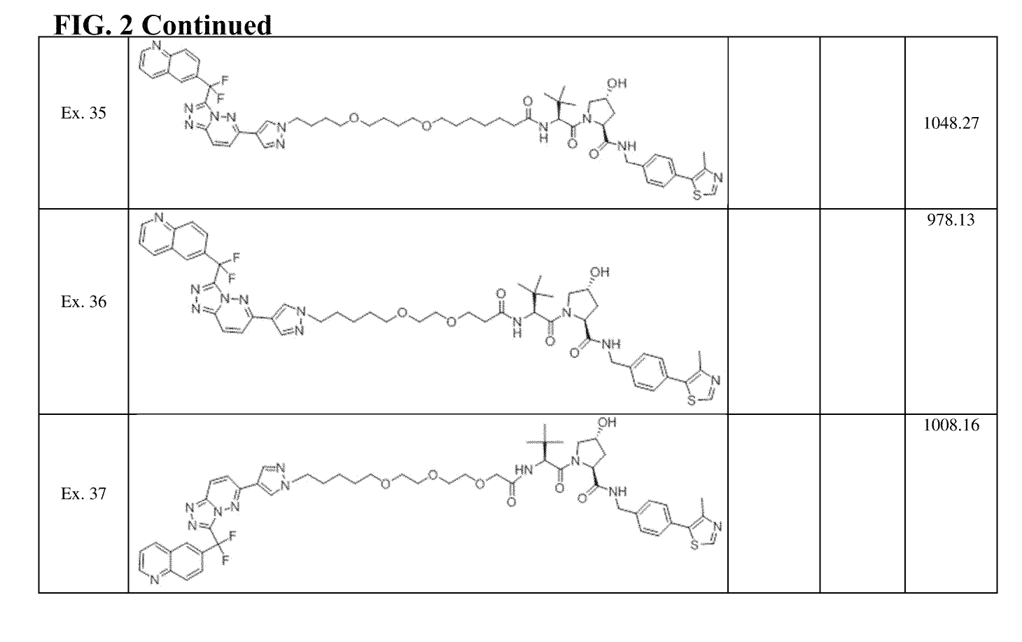

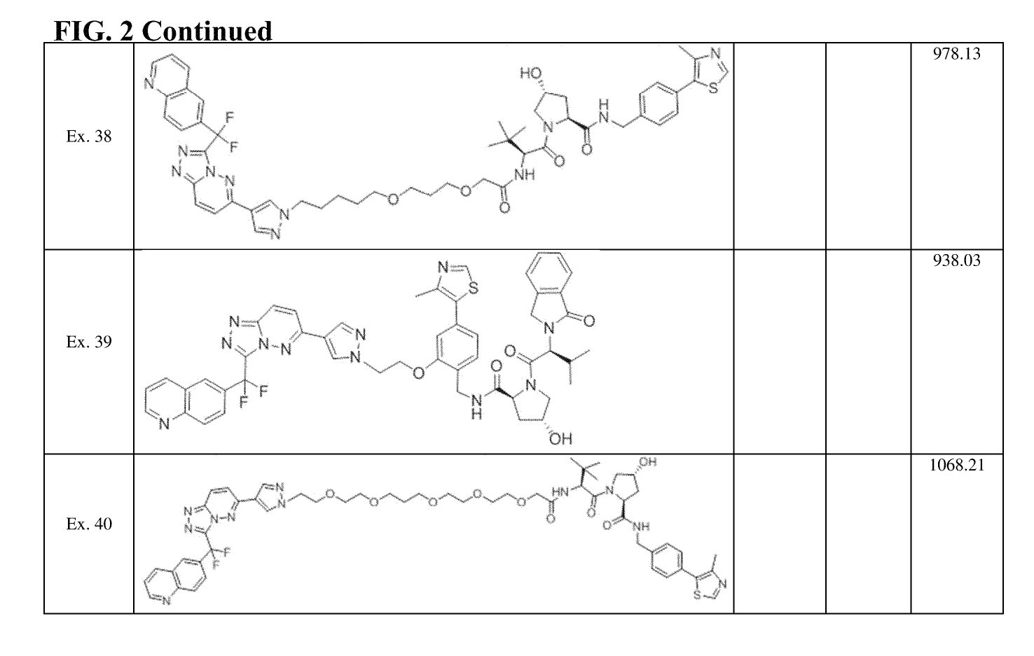

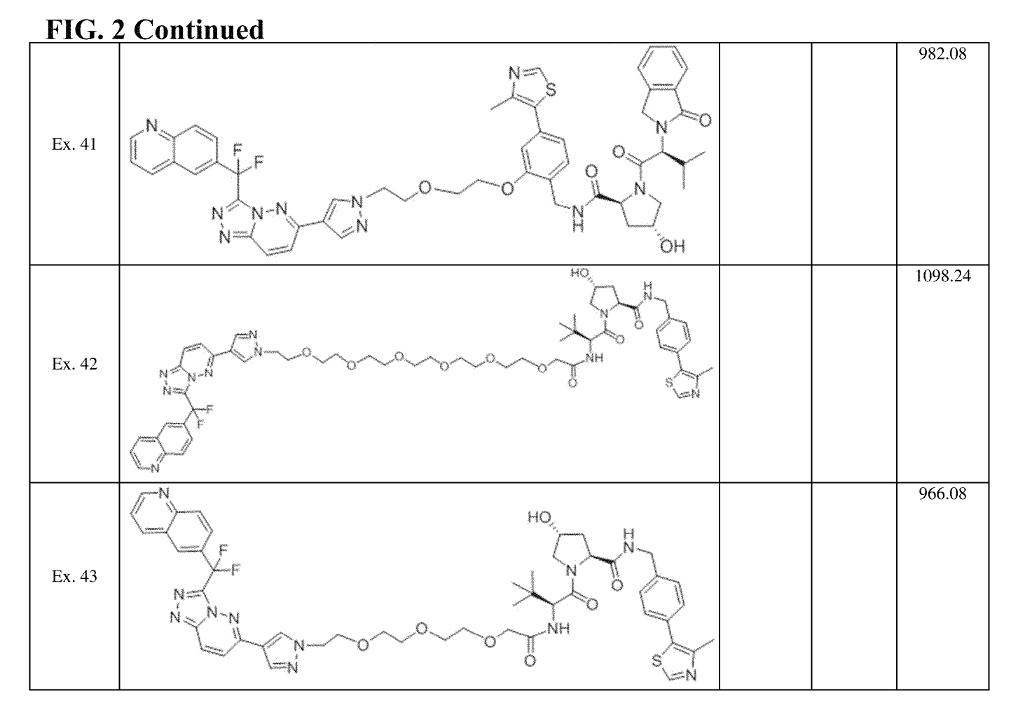

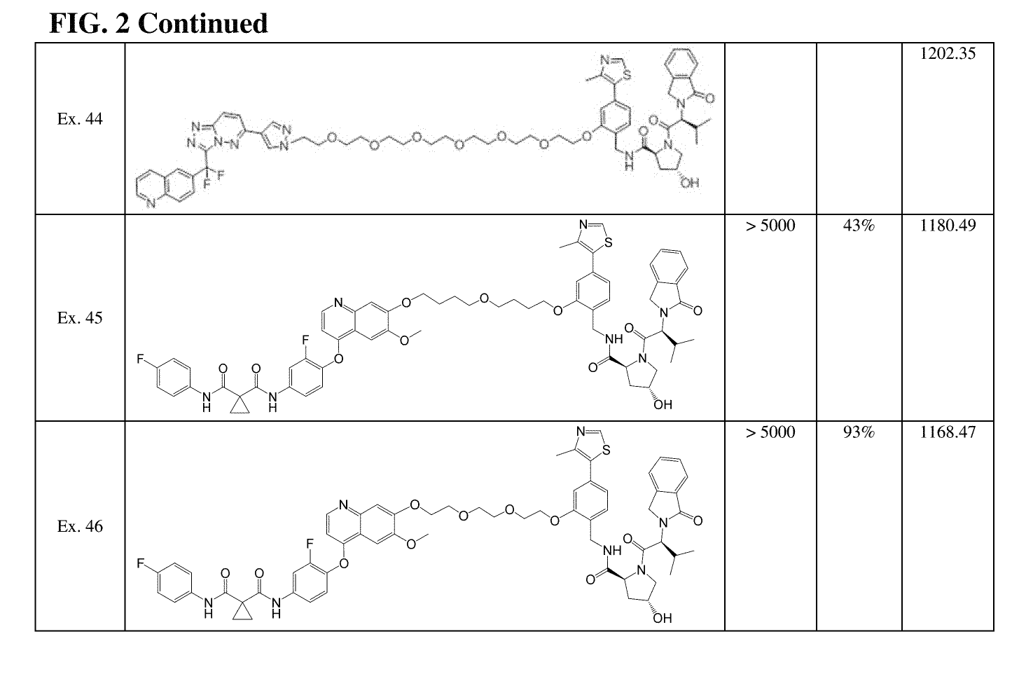

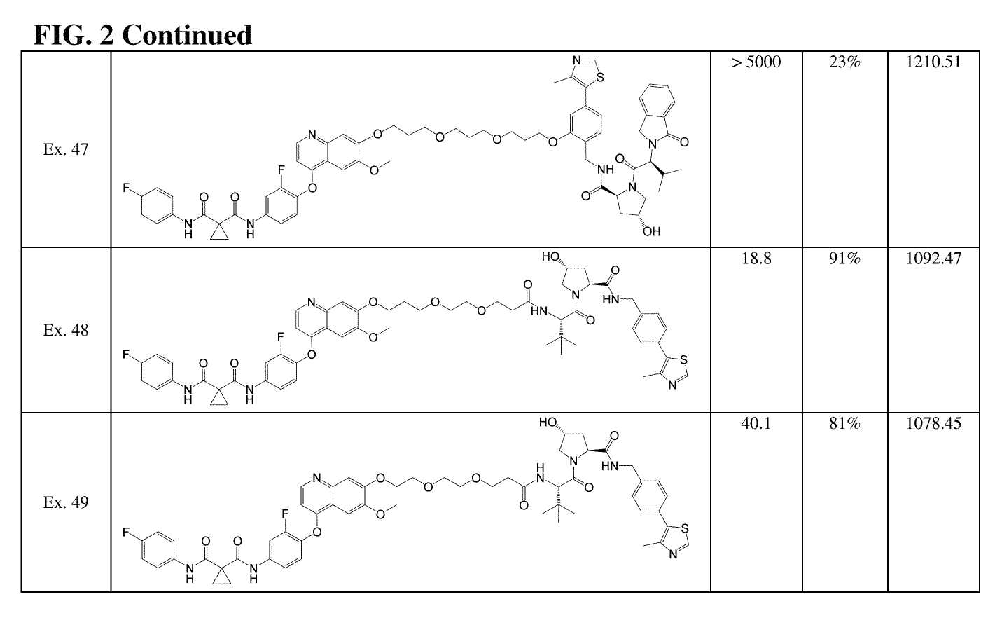

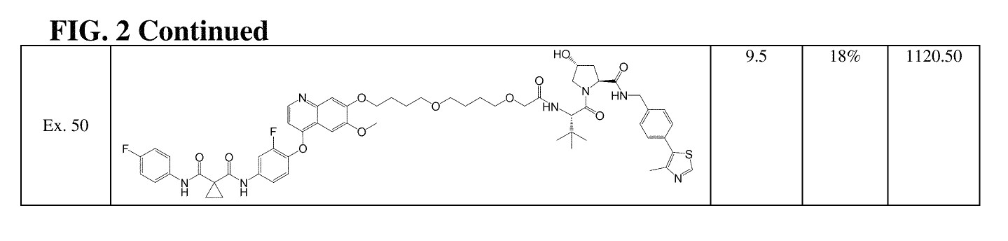

The structure of the following Examples (i.e, Examples 1-12 and 45-50) are shown in Table 1 (see FIG. 2).

### Example 1

To a solution of 3-[3-[3-[[4-[2-fluoro-4-[[1-[(4-fluorophenyl)carbamoyl]cyclopropanecarbonyl]amino]phenoxy]-6-methoxy-7-quinolyl]oxy]propoxy]propoxy]propanoic acid (13.8 mg, 0.02 mmol) and (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide; hydrochloride (11.15 mg, 0.02 mmol) in N,N-Dimethylformamide (2 ml) was added N,N-Diisopropylethylamine (0.17 ml, 0.99 mmol) and 0-(7-Azabenzotriazol-1-yl)-N,N,N′,N′-tetramethyluronium hexafluorophosphate (11.35 mg, 0.03 mmol) at room temperature. The reaction mixture was stirred for 12 hours (overnight) at the same temperature. TLC (DCM:MeOH:NH₄OH, 90:9:1) shows no starting materials. Reaction mixture was diluted with ACOEt (20 mL), washed with water (4×15 mL), dried (Na₂SO₄) and evaporated under vacuum. Crude product was purified by PTLC (DCM:MeOH:NH₄OH, 90:9:1), to give 18 mg of product (82% yield). ¹H NMR (500 MHz, DMSO-d6) δ 10.38 (s, 1H), 10.00 (s, 1H), 8.97 (s, 1H), 8.56 (t, J=6.1 Hz, 1H), 8.46 (d, J=5.2 Hz, 1H), 7.96-7.85 (m, 2H), 7.69-7.59 (m, 2H), 7.51 (d, J=8.8 Hz, 2H), 7.45-7.33 (m, 5H), 7.15 (t, J=8.9 Hz, 2H), 6.41 (d, J=5.1 Hz, 1H), 5.12 (d, J=3.3 Hz, 1H), 4.55 (d, J=9.4 Hz, 1H), 4.43 (ddd, J=10.9, 6.7, 3.3 Hz, 2H), 4.27-4.16 (m, 3H), 3.94 (s, 3H), 3.76-3.33 (m, 10H), 2.58-2.51 (m, 1H), 2.43 (s, 3H), 2.35-2.25 (m, 1H), 2.03 (p, J=5.7 Hz, 3H), 1.95-1.83 (m, 1H), 1.72 (p, J=6.4 Hz, 2H), 1.48 (d, J=3.9 Hz, 4H), 0.92 (s, 9H). LC-MS (ESI); m/z [M+H]⁺: Calcd. for C₅₈H₆₆F₂N₇O₁₁S, 1106.4509. Found 1106.4510.

### Example 2

To a solution of 3-[3-[3-[[4-[2-fluoro-4-[[1-[(4-fluorophenyl)carbamoyl]cyclopropanecarbonyl]amino]phenoxy]-6-methoxy-7-quinolyl]oxy]propoxy]propoxy]propanoic acid (15.7 mg, 0.02 mmol) and 4-(2-aminoethoxy)-2-(2,6-dioxo-3-piperidyl)isoindoline-1,3-dione; 2,2,2-trifluoroacetic acid (11.71 mg, 0.03 mmol) in N,N-Dimethylformamide (2 ml) was added N,N-Diisopropylethylamine (0.2 ml, 1.15 mmol) and O-(7-Azabenzotriazol-1-yl)-N,N,N′,N′-tetramethyluronium hexafluorophosphate (12.91 mg, 0.03 mmol) at room temperature. The reaction mixture was stirred for 12 hours (overnight) at the same temperature. TLC (DCM:MeOH:NH₄OH, 90:9:1) shows no starting materials. Reaction mixture was diluted with ACOEt (20 mL), washed with water (4×15 mL), dried (Na₂SO₄) and evaporated under vacuum. Crude product was purified by PTLC (DCM:MEOH:NH₄OH, 90:9:1), to give 19 mg of product (84% yield). ¹H NMR (400 MHz, DMSO-d6) δ 11.12 (s, 1H), 10.39 (s, 1H), 10.01 (s, 1H), 8.46 (d, J=5.2 Hz, 1H), 8.10 (t, J=5.5 Hz, 1H), 7.90 (dd, J=13.2, 2.4 Hz, 1H), 7.79 (dd, J=8.5, 7.3 Hz, 1H), 7.71-7.55 (m, 2H), 7.58-7.46 (m, 2H), 7.48-7.32 (m, 3H), 7.15 (t, J=8.9 Hz, 2H), 6.41 (d, J=5.0 Hz, 1H), 5.08 (dd, J=12.8, 5.4 Hz, 1H), 4.21 (dt, J=15.8, 6.1 Hz, 4H), 3.95 (s, 3H), 3.63-3.34 (m, 10H), 2.88 (ddd, J=17.1, 14.0, 5.4 Hz, 1H), 2.63-2.43 (m, 2H), 2.31 (t, J=6.3 Hz, 2H), 2.09-1.93 (m, 3H), 1.68 (p, J=6.4 Hz, 2H), 1.53-1.39 (m, 4H). LC-MS (ESI); m/z [M+H]⁺: Calcd. for C₅₁H₅₁F₂N₆O₁₃, 993.3482. Found 993.3468.

### Example 3

To a solution of 3-[4-[4-[[4-[2-fluoro-4-[[1-[(4-fluorophenyl)carbamoyl]cyclopropanecarbonyl]amino]phenoxy]-6-methoxy-7-quinolyl]oxy]butoxy]butoxy]propanoic acid (12.9 mg, 0.02 mmol) and (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide; hydrochloride (10.02 mg, 0.02 mmol) in N,N-Dimethylformamide (2 ml) was added N,N-Diisopropylethylamine (0.2 ml, 1.15 mmol) and 0-(7-Azabenzotriazol-1-yl)-N,N,N′,N′-tetramethyluronium hexafluorophosphate (10.19 mg, 0.03 mmol) at room temperature. The reaction mixture was stirred for 12 hours (overnight) at the same temperature. TLC (DCM:MeOH:NH₄OH, 90:9:1) shows no starting materials. Reaction mixture was diluted with ACOEt (20 mL), washed with water (4×15 mL), dried (Na₂SO₄) and evaporated under vacuum. Crude product was purified by PTLC (DCM:MEOH:NH₄OH, 90:9:1), to give 18 mg of product (80% yield). ¹H NMR (500 MHz, Chloroform-d) δ 10.06 (s, 1H), 8.66 (s, 1H), 8.48 (s, 1H), 8.45 (d, J=5.3 Hz, 1H), 7.75 (dd, J=12.0, 2.4 Hz, 1H), 7.55 (s, 1H), 7.52-7.42 (m, 3H), 7.40 (s, 1H), 7.37-7.29 (m, 4H), 7.30-7.26 (m, 1H), 7.20 (t, J=8.6 Hz, 1H), 7.13 (d, J=8.1 Hz, 1H), 7.05 (t, J=8.6 Hz, 2H), 6.39 (d, J=5.2 Hz, 1H), 4.74 (t, J=7.9 Hz, 1H), 4.59-4.47 (m, 2H), 4.43 (d, J=8.1 Hz, 1H), 4.32 (dd, J=15.0, 5.2 Hz, 1H), 4.21 (t, J=6.7 Hz, 2H), 4.11 (d, J=11.1 Hz, 1H), 4.02 (s, 3H), 3.62 (t, J=5.6 Hz, 2H), 3.57 (dd, J=11.4, 3.6 Hz, 1H), 3.53-3.37 (m, 6H), 2.57-2.50 (m, 1H), 2.50 (s, 3H), 2.44 (t, J=5.6 Hz, 2H), 2.15-2.08 (m, 1H), 2.00 (p, J=7.0 Hz, 2H), 1.84-1.72 (m, 4H), 1.72-1.55 (m, 6H), 0.92 (s, 9H). LC-MS (ESI); m/z [M+H]⁺: Calcd. for C₆₀H₇₀F₂N₇O₁₁S, 1134.4822. Found 1134.4456.

### Example 4

To a solution of 3-[4-[4-[[4-[2-fluoro-4-[[1-[(4-fluorophenyl)carbamoyl]cyclopropanecarbonyl]amino]phenoxy]-6-methoxy-7-quinolyl]oxy]butoxy]butoxy]propanoic acid (14.2 mg, 0.02 mmol) and 4-(2-aminoethoxy)-2-(2,6-dioxo-3-piperidyl)isoindoline-1,3-dione; 2,2,2-trifluoroacetic acid (10.18 mg, 0.024 mmol) in N,N-Dimethylformamide (2 ml) was added N,N-Diisopropylethylamine (0.2 ml, 1.15 mmol) and O-(7-Azabenzotriazol-1-yl)-N,N,N′,N′-tetramethyluronium hexafluorophosphate (11.22 mg, 0.03 mmol) at room temperature. The reaction mixture was stirred for 12 hours (overnight) at the same temperature. TLC (DCM:MeOH:NH₄OH, 90:9:1) shows no starting materials. Reaction mixture was diluted with ACOEt (20 mL), washed with water (4×15 mL), dried (Na₂SO₄) and evaporated under vacuum. Crude product was purified by PTLC (DCM:MEOH:NH₄OH, 90:9:1, 4:6), to give 14 mg of product (70% yield). ¹H NMR (500 MHz, Chloroform-d) δ 9.96 (s, 2H), 8.52 (d, J=5.3 Hz, 1H), 8.42 (s, 1H), 7.75 (dd, J=12.0, 2.5 Hz, 1H), 7.67 (dd, J=8.5, 7.3 Hz, 1H), 7.57 (s, 1H), 7.54 (s, 1H), 7.51-7.41 (m, 3H), 7.30-7.16 (m, 4H), 7.14-7.07 (m, 1H), 7.05 (t, J=8.6 Hz, 2H), 6.39 (d, J=5.3 Hz, 1H), 4.95 (dd, J=12.1, 5.4 Hz, 1H), 4.30-4.14 (m, 4H), 4.02 (s, 3H), 3.78-3.68 (m, 2H), 3.64 (t, J=5.8 Hz, 2H), 3.46-3.37 (m, 4H), 3.36-3.28 (m, 2H), 2.94-2.67 (m, 3H), 2.48 (t, J=5.8 Hz, 2H), 2.13 (dq, J=10.2, 3.6, 3.1 Hz, 1H), 2.04-1.93 (m, 2H), 1.86-1.49 (m, 10H). LC-MS (ESI); m/z [M+H]+: Calcd. for C₅₃H₅₅F₂N₆O₁₃, 1021.3795. Found 1021.2941.

### Example 5

To a mixture of (2S,4R)-4-hydroxy-N-[[2-[3-[2-(3-iodopropoxy)ethoxy]propoxy]-4-(4-methylthiazol-5-yl)phenyl]methyl]-1-[(2S)-3-methyl-2-(1-oxoisoindolin-2-yl)butanoyl]pyrrolidine-2-carboxamide (15.8 mg, 0.02 mmol) and N1′-[3-fluoro-4-[(7-hydroxy-6-methoxy-4-quinolyl)oxy]phenyl]-N1-(4-fluorophenyl)cyclopropane-1,1-dicarboxamide (9.75 mg, 0.02 mmol) in N,N-Dimethylformamide (1 mL) was added Cs₂CO₃ (12.57 mg, 0.04 mmol). After stirring at room temperature for 12 hours (overnight), the reaction mixture was diluted with AcOEt (10 mL) and washed with brine (5×10 mL), organic phase was dried (Na₂SO₄, and evaporated under vacuum. Crude product was purified by PTLC (DCM:MeOH:NH₄OH, 90:9:1) to give 2.5 mg of product (10% yield). ¹H NMR (600 MHz, DMSO-d6) δ 10.39 (s, 1H), 10.01 (s, 1H), 8.96 (s, 1H), 8.45 (d, J=4.8 Hz, 1H), 8.38 (t, J=5.4 Hz, 1H), 7.90 (d, J=13.0 Hz, 1H), 7.70 (d, J=7.5 Hz, 1H), 7.68-7.28 (m, 10H), 7.15 (t, J=8.5 Hz, 2H), 7.04-6.92 (m, 2H), 6.40 (d, J=5.0 Hz, 1H), 5.09 (d, J=3.0 Hz, 1H), 4.70 (d, J=10.8 Hz, 1H), 4.54 (d, J=18.1 Hz, 1H), 4.49-4.37 (m, 2H), 4.35-4.26 (m, 2H), 4.23 (dd, J=16.3, 6.0 Hz, 1H), 4.17 (t, J=6.1 Hz, 2H), 4.09 (t, J=5.7 Hz, 2H), 3.93 (s, 2H), 3.81-3.65 (m, 2H), 3.64-3.46 (m, 8H), 2.45 (s, 3H), 2.35-2.28 (m, 1H), 2.07-1.86 (m, 6H), 1.47 (d, J=5.6 Hz, 4H), 0.95 (d, J=6.2 Hz, 3H), 0.72 (d, J=6.3 Hz, 3H). LC-MS (ESI); m/z: [M+H]⁺ Calcd. for C₆₄H₆₈F₂N₇O₁₂S, 1196.4614. Found 1196.4210.

### Example 6 (Epimer of Example 1)

To a solution of 3-[3-[3-[[4-[2-fluoro-4-[[1-[(4-fluorophenyl)carbamoyl]cyclopropanecarbonyl]amino]phenoxy]-6-methoxy-7-quinolyl]oxy]propoxy]propoxy]propanoic acid (12.9 mg, 0.019 mmol) and (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide; hydrochloride (10.42 mg, 0.022 mmol) in N,N-Dimethylformamide (2 ml) was added N,N-Diisopropylethylamine (0.2 ml, 1.15 mmol) and 0-(7-Azabenzotriazol-1-yl)-N,N,N′,N′-tetramethyluronium hexafluorophosphate (10.6 mg, 0.028 mmol) at room temperature. The reaction mixture was stirred for 12 hours (overnight) at the same temperature. TLC (DCM:MeOH:NH₄OH, 90:9:1) shows no starting materials. Reaction mixture was diluted with ACOEt (20 mL), washed with water (4×15 mL), dried (Na₂SO₄) and evaporated under vacuum. Crude product was purified by PTLC (DCM:MEOH:NH₄OH, 90:9:1), to give 9.7 mg of the expected product (47% yield). ¹H NMR (500 MHz, DMSO-d6) δ 10.38 (s, 1H), 10.00 (s, 1H), 8.97 (s, 1H), 8.63 (t, J=6.0 Hz, 1H), 8.46 (d, J=5.2 Hz, 1H), 7.90 (d, J=9.7 Hz, 2H), 7.71-7.57 (m, 2H), 7.51 (d, J=8.7 Hz, 2H), 7.44-7.28 (m, 5H), 7.15 (t, J=8.9 Hz, 2H), 6.41 (d, J=5.1 Hz, 1H), 5.43 (d, J=7.2 Hz, 1H), 4.56-4.38 (m, 2H), 4.36 (dd, J=8.6, 6.1 Hz, 1H), 4.32-4.13 (m, 4H), 3.94 (s, 3H), 3.97-3.82 (m, 1H), 3.64-3.46 (m, 4H), 3.48-3.35 (m, 4H), 2.57-2.45 (m, 2H), 2.43 (s, 3H), 2.36-2.26 (m, 2H), 2.03 (p, J=6.3 Hz, 2H), 1.73 (dp, J=13.0, 6.2 Hz, 3H), 1.55-1.38 (m, 4H), 0.93 (s, 9H). LC-MS (ESI); m/z [M+H]⁺: Calcd. for C₅₈H₆₆F₂N₇O₁₁S, 1106.4509. Found 1106.5096.

### Example 7 (Epimer of Example 3)

To a solution of 3-[4-[4-[[4-[2-fluoro-4-[[1-[(4-fluorophenyl)carbamoyl]cyclopropanecarbonyl]amino]phenoxy]-6-methoxy-7-quinolyl]oxy]butoxy]butoxy]propanoic acid (16.9 mg, 0.023 mmol) and (2S,4S)-1-[(2S)-2-amino-3,3-dimethyl-butanoyl]-4-hydroxy-N-[[4-(4-methylthiazol-5-yl)phenyl]methyl]pyrrolidine-2-carboxamide; hydrochloride (12.97 mg, 0.028 mmol) in N,N-Dimethylformamide (2 ml) was added N,N-Diisopropylethylamine (0.2 ml, 1.15 mmol) and 0-(7-Azabenzotriazol-1-yl)-N,N,N′,N′-tetramethyluronium hexafluorophosphate (13.2 mg, 0.035 mmol) at room temperature. The reaction mixture was stirred for 12 hours (overnight) at the same temperature. TLC (DCM:MeOH:NH₄OH, 90:9:1) shows no starting materials. Reaction mixture was diluted with ACOEt (20 mL), washed with water (4×15 mL), dried (Na₂SO₄) and evaporated under vacuum. Crude product was purified by PTLC (DCM:MEOH:NH₄OH, 90:9:1), to give 12 mg of product (45% yield). ¹H NMR (500 MHz, Chloroform-d) δ 10.17 (s, 1H), 8.67 (s, 1H), 8.44 (d, J=5.5 Hz, 1H), 8.36 (s, 1H), 7.78 (d, J=14.4 Hz, 1H), 7.73-7.66 (m, 1H), 7.56 (s, 1H), 7.51 (s, 1H), 7.49-7.42 (m, 2H), 7.40-7.31 (m, 4H), 7.29 (ddd, J=8.9, 2.4, 1.2 Hz, 1H), 7.21 (t, J=8.6 Hz, 1H), 7.05 (t, J=8.6 Hz, 2H), 7.00 (d, J=8.7 Hz, 1H), 6.43 (d, J=5.4 Hz, 1H), 4.74 (d, J=9.0 Hz, 1H), 4.62 (dd, J=15.0, 7.0 Hz, 1H), 4.51-4.41 (m, 2H), 4.30 (dd, J=15.0, 5.1 Hz, 1H), 4.22 (t, J=6.7 Hz, 2H), 4.02 (s, 3H), 3.94 (dd, J=11.0, 4.2 Hz, 1H), 3.82-3.76 (m, 1H), 3.64 (td, J=5.7, 2.4 Hz, 2H), 3.45 (dtd, J=27.3, 6.1, 2.7 Hz, 6H), 2.50 (s, 3H), 2.46 (t, J=5.6 Hz, 2H), 2.34 (d, J=14.2 Hz, 1H), 2.23-2.12 (m, 2H), 2.06-1.92 (m, 2H), 1.85-1.72 (m, 4H), 1.70-1.57 (m, 6H), 0.92 (s, 9H). LC-MS (ESI); m/z [M+H]⁺: Calcd. for C₆₀H₇₀F₂N₇O₁₁S, 1134.4822. Found 1134.5804.

### Example 8

To a solution of 3-[3-[3-[[4-[2-fluoro-4-[[1-[(4-fluorophenyl)carbamoyl]cyclopropanecarbonyl]amino]phenoxy-6-methoxy-7-quinolyl]oxy]propoxy]propoxy]propanoic acid (25.3 mg, 0.04 mmol) and 4-(2-aminoethoxy)-2-(1-methyl-2,6-dioxo-3-piperidyl)isoindoline-1,3-dione; 2,2,2-trifluoroacetic acid (16.24 mg, 0.04 mmol) in N,N-Dimethylformamide (2 ml) was added O-(7-Azabenzotriazol-1-yl)-N,N,N′,N′-tetramethyluronium hexafluorophosphate (16.39 mg, 0.04 mmol) and N,N-Diisopropylethylamine (0.29 ml, 1.68 mmol) at room temperature. The reaction mixture was stirred for 4 hours at the same temperature. TLC (DCM:MeOH:NH₄OH, 90:9:1) shows no starting materials. Reaction mixture was diluted with ACOEt (20 mL), washed with water (4×15 mL), dried (Na₂SO₄) and evaporated under vacuum. Crude product was purified by PTLC (DCM:MEOH:NH₄OH, 90:9:1), to give 24 mg of product (65% yield). ¹H NMR (400 MHz, DMSO-d6) δ 10.39 (s, 1H), 10.01 (s, 1H), 8.46 (d, J=5.2 Hz, 1H), 8.08 (t, J=5.1 Hz, 2H), 7.90 (d, J=13.2 Hz, 1H), 7.84-7.74 (m, 1H), 7.69-7.58 (m, 2H), 7.57-7.48 (m, 3H), 7.48-7.31 (m, 3H), 7.15 (t, J=8.8 Hz, 2H), 6.41 (d, J=5.2 Hz, 1H), 5.15 (dd, J=13.0, 5.4 Hz, 1H), 4.21 (dt, J=16.4, 6.3 Hz, 4H), 3.95 (s, 3H), 3.63-3.36 (m, 10H), 3.01 (d, J=1.2 Hz, 3H), 2.97-2.86 (m, 1H), 2.82-2.67 (m, 1H), 2.50 (dt, J=3.5, 1.9 Hz, 1H), 2.32 (t, J=6.4 Hz, 2H), 2.13-1.96 (m, 3H), 1.77-1.61 (m, 2H), 1.48 (bs, 4H). LC-MS (ESI); m/z [M+H]⁺: Calcd. for C₅₂H₅₃F₂N₆O₁₃, 1007.3638. Found 1007.4191.

### Example 9

To a solution of 3-[3-[3-[4-[3-[difluoro(6-quinolyl)methyl]-[1,2,4]triazolo[4,3-b]pyridazin-6-yl]pyrazol-1-yl]propoxy]propoxy]propanoic acid (12.7 mg, 0.023 mmol) and 4-(2-aminoethoxy)-2-(2,6-dioxo-3-piperidyl)isoindoline-1,3-dione; 2,2,2-trifluoroacetic acid (11.9 mg, 0.028 mmol) in N,N-Dimethylformamide (2 ml) was added N,N-Diisopropylethylamine (0.2 ml, 1.15 mmol) and O-(7-Azabenzotriazol-1-yl)-N,N,N′,N′-tetramethyluronium hexafluorophosphate (13.13 mg, 0.033 mmol) at room temperature. The reaction mixture was stirred for 4 hours at the same temperature. TLC (DCM:MeOH:NH₄OH, 90:9:1) shows no starting materials. Reaction mixture was diluted with ACOEt (20 mL), washed with water (4×15 mL), dried (Na₂SO₄) and evaporated under vacuum. Crude product was purified by PTLC (DCM:MEOH:NH₄OH, 90:9:1), to give 12.5 mg of product (64% yield). ¹H NMR (500 MHz, DMSO-d6) δ 11.10 (s, 1H), 9.11-8.90 (m, 1H), 8.61 (d, J=8.3 Hz, 1H), 8.56-8.37 (m, 3H), 8.18 (d, J=8.8 Hz, 1H), 8.13-7.92 (m, 3H), 7.86-7.72 (m, 2H), 7.64 (dd, J=8.2, 4.2 Hz, 1H), 7.51 (d, J=8.5 Hz, 1H), 7.44 (d, J=7.2 Hz, 1H), 5.08 (dd, J=12.9, 5.4 Hz, 1H), 4.31-4.16 (m, 4H), 3.64-3.26 (m, 10H), 2.96-2.80 (m, 1H), 2.67-2.42 (m, 2H), 2.31 (t, J=6.4 Hz, 2H), 2.09-1.93 (m, 3H), 1.72-1.60 (m, 2H). LC-MS (ESI); m/z [M+H]⁺: Calcd. for C₄₃H₄₁F₂N₁₀O₈, 851.3076. Found 851.3187.

### Example 10

To a solution of 3-[3-[3-[4-[3-[difluoro(6-quinolyl)methyl]-[1,2,4]triazolo[4,3-b]pyridazin-6-yl]pyrazol-1-yl]propoxy]propoxy]propanoic acid (12.7 mg, 0.023 mmol) and (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide; hydrochloride (12.9 mg, 0.03 mmol) in N,N-Dimethylformamide (2 ml) was added N,N-Diisopropylethylamine (0.2 ml, 1.15 mmol) and O-(7-Azabenzotriazol-1-yl)-N,N,N′,N′-tetramethyluronium hexafluorophosphate (13.13 mg, 0.033 mmol) at room temperature. The reaction mixture was stirred for 4 hours at the same temperature. TLC (DCM:MeOH:NH₄OH, 90:9:1) shows no starting materials. Reaction mixture was diluted with ACOEt (20 mL), washed with water (4×15 mL), dried (Na₂SO₄) and evaporated under vacuum. Crude product was purified by PTLC (DCM:MEOH:NH₄OH, 90:9:1), to give 18 mg of product (82% yield). ¹H NMR (500 MHz, DMSO-d6) δ 9.02 (dd, J=4.3, 1.7 Hz, 1H), 8.97 (s, 1H), 8.65-8.44 (m, 6H), 8.18 (d, J=8.8 Hz, 1H), 8.05 (s, 1H), 8.02 (dd, J=8.9, 2.1 Hz, 1H), 7.88 (d, J=9.4 Hz, 1H), 7.83 (d, J=9.7 Hz, 1H), 7.64 (dd, J=8.3, 4.2 Hz, 1H), 7.45-7.32 (m, 4H), 5.12 (d, J=3.5 Hz, 1H), 4.55 (d, J=9.4 Hz, 1H), 4.42 (ddd, J=11.1, 6.8, 3.4 Hz, 2H), 4.37-4.32 (m, 1H), 4.21 (q, J=6.7 Hz, 3H), 3.71-3.31 (m, 11H), 2.61-2.49 (m, 1H), 2.43 (s, 3H), 2.31 (dt, J=14.6, 6.0 Hz, 1H), 2.02 (h, J=7.5, 6.8 Hz, 3H), 1.90 (ddd, J=12.9, 8.6, 4.8 Hz, 1H), 1.69 (p, J=6.5 Hz, 2H), 0.91 (s, 9H). LC-MS (ESI); m/z [M+H]⁺: Calcd. for C₄₉H₅₆F₂N₁₁O₆S, 964.4103. Found 964.4097.

### Example 11

To a solution of 3-[4-[4-[4-[3-[difluoro(6-quinolyl)methyl]-[1,2,4]triazolo[4,3-b]pyridazin-6-yl]pyrazol-1-yl]-butoxy]butoxy]propanoic acid (15 mg, 0.03 mmol) and 4-(2-aminoethoxy)-2-(2,6-dioxo-3-piperidyl)isoindoline-1,3-dione; 2,2,2-trifluoroacetic acid (13.39 mg, 0.03 mmol) in N,N-Dimethylformamide (2 ml) was added N,N-Diisopropylethylamine (0.2 ml, 1.15 mmol) and O-(7-Azabenzotriazol-1-yl)-N,N,N′,N′-tetramethyluronium hexafluorophosphate (14.76 mg, 0.04 mmol) at room temperature. The reaction mixture was stirred for 4 hours at the same temperature. TLC (DCM:MeOH:NH₄OH, 90:9:1) shows no starting materials. Reaction mixture was diluted with AcOEt (20 mL), washed with water (4×15 mL), dried (Na₂SO₄) and evaporated under vacuum. Crude product was purified by PTLC (DCM:MEOH:NH₄OH, 90:9:1), to give 17 mg of product (74% yield). ¹H NMR (500 MHz, Chloroform-d) δ 9.08 (s, 1H), 9.02 (d, J=3.6 Hz, 1H), 8.33-8.21 (m, 3H), 8.11 (d, J=9.7 Hz, 1H), 8.11-8.02 (m, 1H), 7.97 (d, J=8.1 Hz, 2H), 7.67 (t, J=7.9 Hz, 1H), 7.54-7.43 (m, 2H), 7.40 (d, J=9.7 Hz, 1H), 7.23 (d, J=8.5 Hz, 1H), 6.99 (t, J=5.5 Hz, 1H), 4.94 (dd, J=12.2, 5.2 Hz, 1H), 4.22 (t, J=6.8 Hz, 4H), 3.82-3.57 (m, 4H), 3.55-3.27 (m, 6H), 2.97-2.66 (m, 3H), 2.47 (t, J=6.0 Hz, 2H), 2.20-2.07 (m, 1H), 1.99 (p, J=7.3 Hz, 2H), 1.80-1.48 (m, 8H). LC-MS (ESI); m/z [M+H]⁺: Calcd. for C₄₄H₄₅F₂N₁₀O₈, 879.3389. Found 879.3406.

### Example 12

To a solution of 3-[4-[4-[4-[3-[difluoro(6-quinolyl)methyl]-[1,2,4]triazolo[4,3-b]pyridazin-6-yl]pyrazol-1-yl]butoxy]butoxy]propanoic acid (23 mg, 0.04 mmol) and (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide; hydrochloride (22.24 mg, 0.05 mmol) in N,N-Dimethylformamide (2 ml) was added N,N-Diisopropylethylamine (0.2 ml, 1.15 mmol) and O-(7-Azabenzotriazol-1-yl)-N,N,N′,N′-tetramethyluronium hexafluorophosphate (22.63 mg, 0.06 mmol) at room temperature. The reaction mixture was stirred for 4 hours at the same temperature. TLC (DCM:MeOH:NH₄OH, 90:9:1) shows no starting materials. Reaction mixture was diluted with ACOEt (20 mL), washed with water (4×15 mL), dried (Na₂SO₄) and evaporated under vacuum. Crude product was purified by PTLC (DCM:MEOH:NH₄OH, 90:9:1), to give 29 mg of product (74% yield). ¹H NMR (500 MHz, Chloroform-d) δ 8.99 (d, J=3.9 Hz, 1H), 8.66 (s, 1H), 8.33-8.20 (m, 3H), 8.13 (d, J=9.7 Hz, 1H), 8.09-8.03 (m, 1H), 7.97 (s, 2H), 7.52-7.45 (m, 1H), 7.46-7.37 (m, 2H), 7.37-7.28 (m, 4H), 7.01 (d, J=8.2 Hz, 1H), 4.72 (t, J=8.0 Hz, 1H), 4.60-4.48 (m, 2H), 4.44 (d, J=8.3 Hz, 1H), 4.32 (dd, J=15.0, 5.2 Hz, 1H), 4.21 (t, J=7.1 Hz, 2H), 4.10 (d, J=11.4 Hz, 1H), 3.67-3.52 (m, 3H), 3.42 (ddd, J=19.9, 13.6, 5.8 Hz, 6H), 2.61-2.48 (m, 1H), 2.49 (s, 3H), 2.43 (dd, J=6.6, 4.8 Hz, 2H), 2.16-2.07 (m, 1H), 2.04-1.93 (m, 2H), 1.70-1.51 (m, 6H), 0.92 (s, 9H). LC-MS (ESI); m/z [M+H]⁺: Calcd. for C₅₁H₆₀F₂N₁₁O₆S, 992.4416. Found 992.4521.

### Example 45

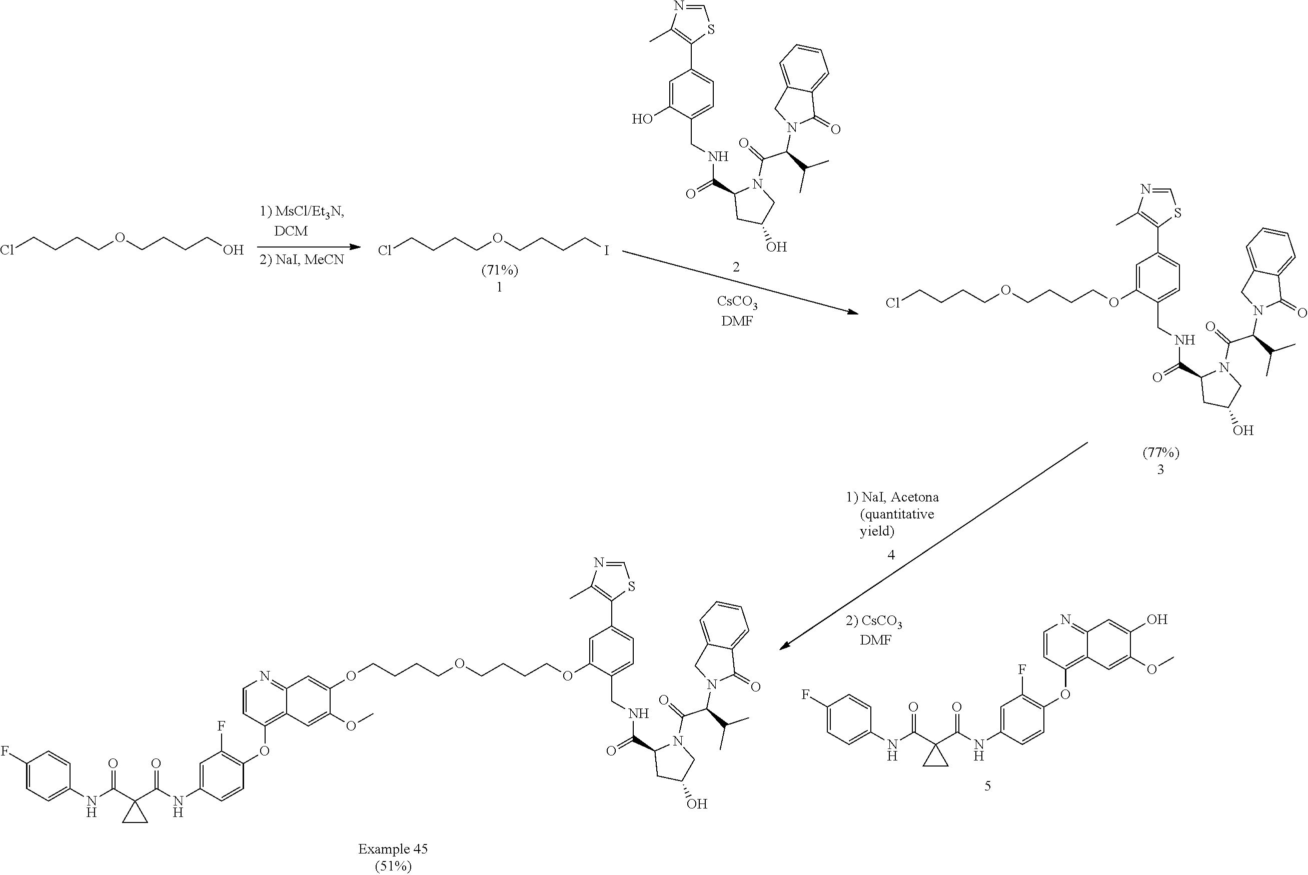 [SMILES: OCCCCOCCCCCl ; ClCCCCOCCCCI ; Cc1ncsc1-c1ccc(CNC(=O)[C@@H]2C[C@@H](O)CN2C(=O)[C@H](C(C)C)N2Cc3ccccc3C2=O)c(O)c1 ; Cc1ncsc1-c1ccc(CNC(=O)[C@@H]2C[C@@H](O)CN2C(=O)[C@H](C(C)C)N2Cc3ccccc3C2=O)c(OCCCCOCCCCCl)c1 ; COc1cc2c(Oc3ccc(NC(=O)C4(C(=O)Nc5ccc(F)cc5)CC4)cc3F)ccnc2cc1OCCCCOCCCCOc1cc(-c2scnc2C)ccc1CNC(=O)[C@@H]1C[C@@H](O)CN1C(=O)[C@H](C(C)C)N1Cc2ccccc2C1=O ; COc1cc2c(Oc3ccc(NC(=O)C4(C(=O)Nc5ccc(F)cc5)CC4)cc3F)ccnc2cc1O]

### 1-chloro-4-(4-iodobutoxy)butane (1)

To a solution of 4-(4-chlorobutoxy)butan-1-ol (271 mg, 1.5 mmol) in Dichloromethane (5 ml) was added TEA (0.63 ml, 4.5 mmol), then reaction mixture was cooled to 0° C. (water ice/acetone bath) and Mesyl chloride (0.14 ml, 1.8 mmol) was added dropwise. The reaction mixture was stirred for 1 h at the same temperature. By TLC no starting material (Hex:AcOEt, 3:7), and a less polar compound was formed. Reaction mixture was poured into an aqueous solution of NaHCO₃ (20 mL) and product extracted with DCM (20 mL, 2×), the organic extracts were combined, dried (Na₂SO₄), and evaporated under vacuum. The crude product (mesylate) was used in the next step without any further purification (>95% pure by NMR); ¹H NMR (400 MHz, Chloroform-d) δ 4.26 (t, J=6.5 Hz, 2H), 3.57 (t, J=6.6 Hz, 2H), 3.44 (td, J=6.2, 2.0 Hz, 4H), 3.01 (s, 3H), 1.92-1.78 (m, 4H), 1.76-1.62 (m, 4H). Crude mixture from previous step was diluted in Acetonitrile (5 ml) and NaI (247.32 mg, 1.65 mmol) was added, the reaction mixture was stirred at room temperature for 72 h. The reaction mixture was poured into an aqueous solution of Na₂S₂O₃ (10%, 20 mL) and product was extracted with DCM (2×20 mL). Organic extracts were combined, dried (NA₂SO₄) and evaporated under vacuum. Crude product was purified by flash chromatography (SiO₂-40 g, Grad. Hex:AcOEt, 2 to 20% in 10 min), to give 310 mg of product as an oil (71% yield). ¹H NMR (500 MHz, Chloroform-d) δ 3.57 (t, J=6.6 Hz, 2H), 3.43 (td, J=6.3, 2.4 Hz, 4H), 3.22 (t, J=7.0 Hz, 2H), 2.01-1.80 (m, 4H), 1.77-1.61 (m, 4H). ¹³C NMR (151 MHz, cdcl3) δ 70.13, 69.77, 45.13, 30.74, 30.56, 29.68, 27.22, 7.02. LC-MS (ESI); m/z: [M+H]⁺ Calcd. for C₈H₁₇ClO, 291.001. Found 291.0060.

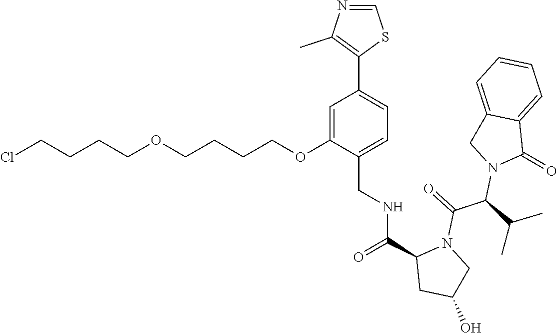 [SMILES: Cc1ncsc1-c1ccc(CNC(=O)[C@@H]2C[C@@H](O)CN2C(=O)[C@H](C(C)C)N2Cc3ccccc3C2=O)c(OCCCCOCCCCCl)c1]

### (2S,4R)—N-(2-(4-(4-chlorobutoxy)butoxy)-4-(4-methylthiazol-5-yl)benzyl)-4-hydroxy-1-((S)-3-methyl-2-(1-oxoisoindolin-2-yl)butanoyl)pyrrolidine-2-carboxamide (3)

To a mixture of (2S,4R)-4-hydroxy-N-[[2-hydroxy-4-(4-methylthiazol-5-yl)phenyl]methyl]-1[(2S)-3-methyl-2-(1-oxoisoindo-lin-2-yl)butanoyl]pyrrolidine-2-carboxamide (22 mg, 0.04 mmol) (2) and 1-chloro-4-(4-iodobutoxy)butane (13.98 mg, 0.05 mmol) in N,N-Dimethylformamide (1 mL) was added Cs₂CO₃ (26.13 mg, 0.08 mmol). After stirring at room temperature for 2 hrs, the reaction mixture was diluted with AcOEt (10 mL) and washed with water (5×10 mL), organic phase was dried (Na₂SO₄, and evaporated under vacuum. Crude product was purified by PTLC (DCM:MB, 1:1) to give 25 mg of product (88% yield). ¹H NMR (500 MHz, DMSO-d6) δ 8.99 (s, 1H), 8.36 (t, J=5.7 Hz, 1H), 7.71 (d, J=7.5 Hz, 1H), 7.65-7.57 (m, 2H), 7.50 (t, J=7.7 Hz, 1H), 7.33 (d, J=7.6 Hz, 1H), 7.02-6.96 (m, 2H), 5.08 (d, J=3.9 Hz, 1H), 4.71 (d, J=10.8 Hz, 1H), 4.59-4.18 (m, 6H), 4.07 (t, J=5.0 Hz, 2H), 3.77 (dd, J=10.5, 4.2 Hz, 1H), 3.69 (d, J=10.4 Hz, 1H), 3.63 (t, J=6.6 Hz, 2H), 3.49-3.36 (m, 4H), 2.47 (s, 3H), 2.37-2.29 (m, 1H), 2.07-2.01 (m, 1H), 1.96-1.89 (m, 1H), 1.85-1.56 (m, 8H), 0.96 (d, J=6.4 Hz, 3H), 0.74 (d, J=6.6 Hz, 3H). ¹³C NMR (151 MHz, dmso) δ 171.54, 168.09, 167.48, 155.90, 151.48, 147.89, 142.21, 131.60, 131.37, 131.32, 130.98, 127.92, 127.69, 126.96, 123.63, 123.02, 120.77, 111.69, 69.68, 69.14, 68.62, 67.56, 58.70, 57.79, 55.42, 46.82, 45.36, 38.10, 37.06, 29.18, 28.40, 26.63, 25.90, 25.69, 18.89, 18.64, 16.03. LC-MS (ESI); m/z: [M+H]⁺ Calcd. for C₃₇H₄₈C1N₄O₆S, 711.2983. Found 711.3224.

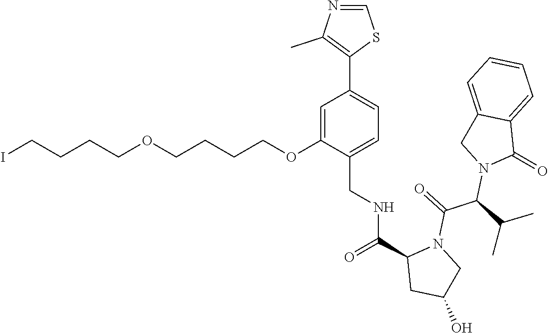 [SMILES: Cc1ncsc1-c1ccc(CNC(=O)[C@@H]2C[C@@H](O)CN2C(=O)[C@H](C(C)C)N2Cc3ccccc3C2=O)c(OCCCCOCCCCI)c1]

### (2S,4R)-4-hydroxy-N-[[2-[4-(4-iodobutoxy)butoxy]-4-(4-methylthiazol-5-yl)phenyl]methyl]-1-[(2S)-3-methyl-2-(1-oxoisoindolin-2-yl)butanoyl]pyrrolidine-2-carboxamide (4)

To a solution of (2S,4R)—N-[[2-[4-(4-chlorobutoxy)butoxy]-4-(4-methylthiazol-5-yl)phenyl]methyl]-4-hydroxy-1-[(2S)-3-methyl-2-(1-oxoisoindolin-2-yl)butanoyl]pyrrolidine-2-carboxamide (3) (23 mg, 0.03 mmol) in Acetone (5 ml) was added NaI (48.47 mg, 0.32 mmol). The reaction mixture was stirred at reflux temperature for 24 h, then the solvent was removed under vacuum and crude product was dissolved in EtOAc (15 mL) and an aqueous solution of Na₂SO₃ (10%, 10 mL), organic layer was separated, washed with water (10 mL), dried (Na₂SO₄) and evaporated under vacuum. Crude product was pure by NMR (>95% purity, 22 mg, 84% yield) no further purification. ¹H (400 MHz, Chloroform-d) δ 8.68 (s, 1H), 7.78 (d, J=7.6 Hz, 1H), 7.52 (t, J=7.4 Hz, 1H), 7.47-7.37 (m, 2H), 7.34-7.23 (m, 2H), 6.96 (d, J=8.7 Hz, 1H), 6.87 (s, 1H), 4.76 (s, 1H), 4.73 (d, J=6.7 Hz, 1H), 4.64 (t, J=7.8 Hz, 1H), 4.57-4.36 (m, 5H), 4.05 (t, J=6.2 Hz, 2H), 3.65 (dd, J=11.5, 3.5 Hz, 1H), 3.49 (t, J=6.2 Hz, 2H), 3.44 (t, J=6.2 Hz, 2H), 3.19 (t, J=6.9 Hz, 2H), 2.58-2.45 (m, 1H), 2.53 (s, 3H), 2.47-2.27 (m, 2H), 2.12-2.00 (m, 2H), 1.98-1.84 (m, 3H), 1.79 (dt, J=9.4, 6.5 Hz, 2H), 1.71-1.58 (m, 2H), 0.89 (dd, J=6.0 Hz, 6H). ¹³C NMR (126 MHz, cdcl3) δ 170.54, 170.47, 169.71, 156.94, 150.41, 148.61, 142.23, 132.35, 131.99, 131.92, 131.73, 129.41, 128.15, 126.47, 123.97, 122.99, 121.65, 112.15, 70.62, 70.13, 69.83, 68.04, 58.81, 58.55, 56.07, 47.59, 39.05, 35.87, 30.75, 30.58, 29.85, 28.89, 26.55, 26.34, 19.23, 16.28, 6.98. LC-MS (ESI); m/z [M+H]⁺: Calcd. for C₃₇H₄₈IN₄O₆S, 803.2339. Found 803.2675.

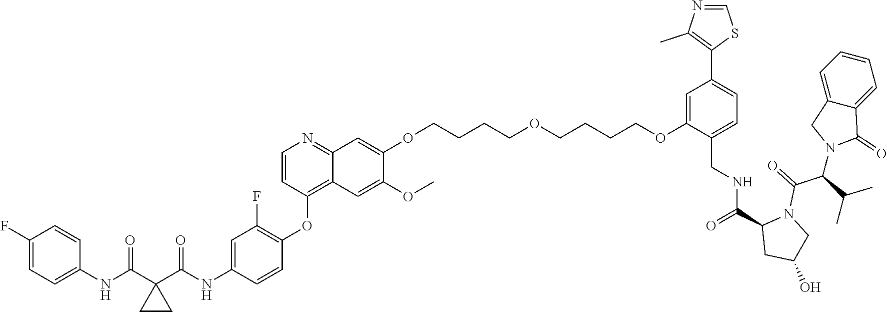 [SMILES: COc1cc2c(Oc3ccc(NC(=O)C4(C(=O)Nc5ccc(F)cc5)CC4)cc3F)ccnc2cc1OCCCCOCCCCOc1cc(-c2scnc2C)ccc1CNC(=O)[C@@H]1C[C@@H](O)CN1C(=O)[C@H](C(C)C)N1Cc2ccccc2C1=O]

### N-(3-fluoro-4-((7-(4-(4-(2-(((2S,4R)-4-hydroxy-1-48)-3-methyl-2-(1-oxoisoindolin-2-yl)butano-yl)pyrrolidine-2-carboxamido)methyl)-5-(4-methylthiazol-5-yl)phenoxy)butoxy)butoxy)-6-methoxyquinolin-4-yl)oxy)phenyl)-N-(4-fluorophenyl)cyclopropane-1,1-dicarboxamide (PROTAC 45)

To a mixture of compound (4) (7.5 mg, 0.01 mmol) and N1′-[3-fluoro-4-[(7-hydroxy-6-methoxy-4-quinolyl)oxy]phenyl]-N1-(4-fluorophenyl)cyclopropane-1,1-dicarboxamide (5) (4.72 mg, 0.01 mmol) in N,N-Dimethylformamide (1 mL) was added Cs₂CO₃ (6.09 mg, 0.02 mmol). After stirring at room temperature for 12 hrs (overnight), the reaction mixture was diluted with AcOEt (10 mL) and washed with brine (5×10 mL), organic phase was dried (Na₂SO₄, and evaporated under vacuum. Crude product was purified by PTLC (DCM:MeOH:NH₄OH, 90:9:1) to give 5.7 mg of product (51% yield). ¹H NMR (400 MHz, DMSO-d6) δ 10.38 (s, 1H), 10.01 (s, 1H), 8.97 (s, 1H), 8.45 (d, J=5.2 Hz, 1H), 8.37 (t, J=6.0 Hz, 1H), 7.90 (dd, J=13.2, 2.4 Hz, 1H), 7.71 (d, J=7.6 Hz, 1H), 7.65-7.38 (m, 8H), 7.34 (d, J=7.6 Hz, 1H), 7.15 (t, J=8.9 Hz, 2H), 7.04-6.87 (m, 2H), 6.41 (d, J=5.2 Hz, 1H), 5.09 (d, J=3.9 Hz, 1H), 4.71 (d, J=10.8 Hz, 1H), 4.50 (dd, 2H), 4.44-4.19 (m, 4H), 4.16 (t, J=6.3 Hz, 2H), 4.07 (t, J=6.0 Hz, 2H), 3.77 (dd, J=10.5, 4.2 Hz, 1H), 3.69 (d, J=10.5 Hz, 1H), 3.47 (t, J=6.3 Hz, 4H), 2.46 (s, 3H), 2.37-2.28 (m, 1H), 2.09-2.00 (m, 1H), 1.96-1.63 (m, 8H), 1.58-1.38 (m, 4H), 0.96 (d, J=6.4 Hz, 3H), 0.72 (d, J=6.6 Hz, 3H). ¹³C NMR (151 MHz, DMSO-d6) δ 171.53, 168.29, 168.09, 167.90, 167.47, 159.35, 158.29 (d, J=240.3 Hz), 155.91, 153.26 (d, J=245.1 Hz), 151.98, 151.43, 149.59, 148.74, 147.88, 146.31, 142.20, 138.02 (d, J=9.9 Hz), 135.65 (d, J=12.3 Hz), 135.20 (d, J=2.4 Hz), 131.58, 131.34 (d, J=8.6 Hz), 130.98, 127.90, 127.70, 126.98, 123.82, 123.61, 123.01, 122.45 (d, J=7.7 Hz), 120.76, 116.92, 115.04 (d, J=22.2 Hz), 114.42, 111.69, 108.97 (d, J=23.0 Hz), 108.39, 101.91, 98.98, 69.70, 69.63, 68.63, 68.18, 67.57, 58.70, 57.78, 55.78, 55.43, 46.81, 38.11, 37.06, 31.93, 28.40, 25.97, 25.73, 25.48, 18.88, 18.62, 16.02, 15.34. LC-MS (ESI); m/z: [M+H]⁺ Calcd. for C₆₄H₆₈F₂N₇O₁₁S, 1180.4665. Found 1180.4974.

### Example 46

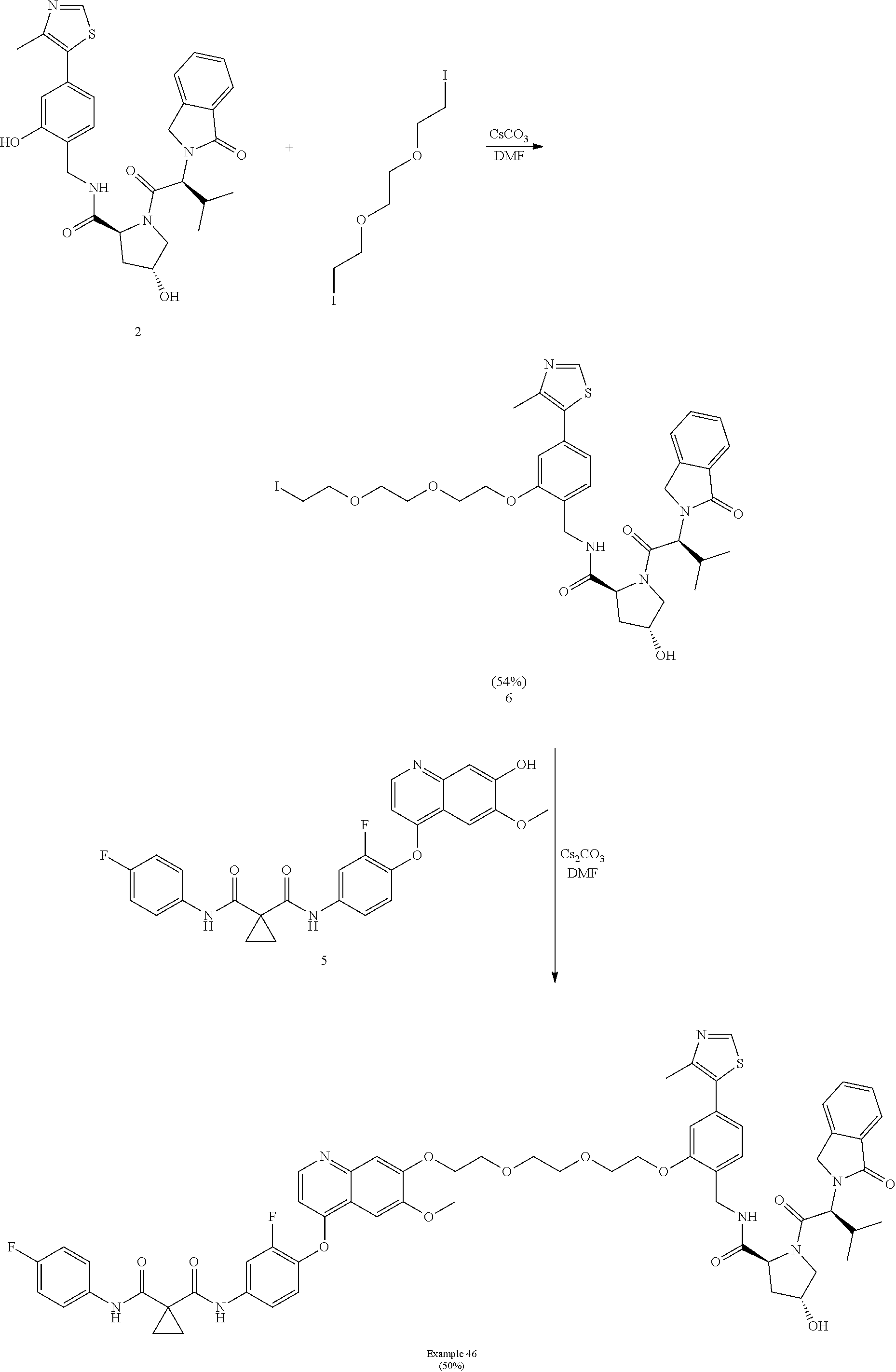

### (2S,4R)-4-hydroxy-N-(2-(2-(2-(2-iodoethoxy)ethoxy)ethoxy)-4-(4-methylthiazol-5-yl)benzyl)-1-((S)-3-methyl-2-(1-oxoisoindolin-2-yl)butanoyl)pyrrolidine-2-carboxamide (6)

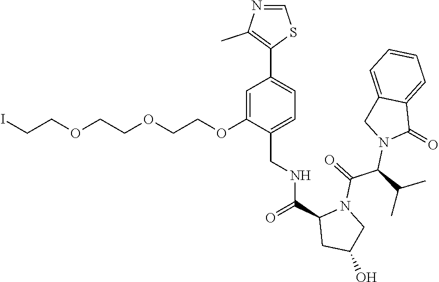 [SMILES: Cc1ncsc1-c1ccc(CNC(=O)[C@@H]2C[C@@H](O)CN2C(=O)[C@H](C(C)C)N2Cc3ccccc3C2=O)c(OCCOCCOCCI)c1]

To a mixture of (2S,4R)-4-hydroxy-N-[[2-hydroxy-4-(4-methylthiazol-5-yl)phenyl]methyl]-1-[(2S)-3-methyl-2-(1-oxoisoindolin-2-yl)butanoyl]pyrrolidine-2-carboxamide (2) (37 mg, 0.07 mmol) and 1,2-bis(2-iodoethoxy)ethane (324.35 mg, 0.88 mmol) in N,N-Dimethylformamide (1 mL) was added Cs₂CO₃ (142.82 mg, 0.44 mmol). After stirring at room temperature for 2 hrs, the reaction mixture was diluted with AcOEt (10 mL) and washed with water (5×10 mL), organic phase was dried (Na₂SO₄), and evaporated under vacuum. Crude product was filtered over a short column of SiO₂ (gradient, DCM 100% to DCM:MeOH:NH₄OH, 90:9:1) to remove the excess of the bis-iodo reactant, then crude product was purified by PTLC (DCM:MeOH:NH₄OH, 90:9:1) to give 29 mg of product (54% yield). ¹H NMR (400 MHz, DMSO-d6) δ 8.98 (s, 1H), 8.35 (t, J=5.7 Hz, 1H), 7.71 (d, J=7.5 Hz, 1H), 7.66-7.56 (m, 2H), 7.49 (t, J=7.3 Hz, 1H), 7.34 (d, J=7.8 Hz, 1H), 7.05 (bs, 1H), 7.01 (d, J=7.8 Hz, 1H), 5.09 (bs, 1H), 4.72 (d, J=10.8 Hz, 1H), 4.64-4.02 (m, 8H), 3.91-3.49 (m, 10H), 3.29 (t, J=6.2 Hz, 2H), 2.47 (s, 3H), 2.40-2.24 (m, 1H), 2.10-1.98 (m, 1H), 1.99-1.85 (m, 1H), 0.97 (d, J=6.4 Hz, 3H), 0.74 (d, J=6.5 Hz, 3H). ¹³C NMR (151 MHz, dmso) δ 171.59, 168.12, 167.52, 155.88, 151.50, 147.95, 142.21, 131.61, 131.38, 131.29, 130.99, 127.93, 127.70, 127.22, 123.64, 123.04, 121.11, 112.19, 71.01, 70.12, 69.41, 69.05, 68.66, 67.96, 58.75, 57.82, 55.46, 46.86, 38.14, 37.16, 28.43, 18.93, 18.66, 16.08, 5.46. LC-MS (ESI); m/z: [M+H]⁺ Calcd. for C₃₅H₄₄IN₄O₇S, 791.1975. Found 791.2036.

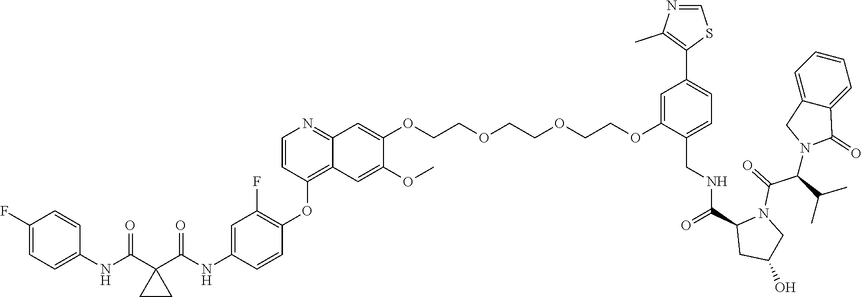 [SMILES: COc1cc2c(Oc3ccc(NC(=O)C4(C(=O)Nc5ccc(F)cc5)CC4)cc3F)ccnc2cc1OCCOCCOCCOc1cc(-c2scnc2C)ccc1CNC(=O)[C@@H]1C[C@@H](O)CN1C(=O)[C@H](C(C)C)N1Cc2ccccc2C1=O]

### N-(3-fluoro-4-((7-(2-(2-(2-(2-(((2S,4R)-4-hydroxy-1-((S)-3-methyl-2-(1-oxoisoindolin-2-yl)-butanoyl)pyrrolidine-2-carboxamido)methyl)-5-(4-methylthiazol-5-yl)phenoxy)ethoxy)ethoxy)-ethoxy)-6-methoxyquinolin-4-yl)oxy)phenyl)-N-(4-fluorophenyl)cyclopropane-1,1-dicarboxamide (PROTAC 46)

To a mixture of compound (6) and N1′-[3-fluoro-4-[(7-hydroxy-6-methoxy-4-quinolyl)oxy]phenyl]-N1-(4-fluorophenyl)cyclopropane-1,1-dicarboxamide (5) (4.72 mg, 0.01 mmol) in N,N-Dimethylformamide (1 mL) was added Cs₂CO₃ (6.09 mg, 0.02 mmol). After stirring at room temperature for 12 hrs (overnight), the reaction mixture was diluted with AcOEt (10 mL) and washed with brine (5×10 mL), organic phase was dried (Na₂SO₄), and evaporated under vacuum. Crude product was purified by PTLC (DCM:MeOH, 9:1 2×) to give 9.7 mg of product (50% yield). ¹H NMR (400 MHz, DMSO-d6) δ 10.39 (bs, 1H), 10.01 (bs, 1H), 8.97 (s, 1H), 8.46 (d, J=4.8 Hz, 1H), 8.37 (bs, 1H), 7.90 (d, J=12.9 Hz, 1H), 7.81-7.26 (m, 10H), 7.15 (t, J=8.7 Hz, 2H), 7.10-6.94 (m, 2H), 6.41 (d, J=4.7 Hz, 1H), 5.09 (d, 1H), 4.71 (d, J=10.7 Hz, 1H), 4.59-4.11 (m, 10H), 3.94 (s, 3H), 3.90-3.45 (m, 10H), 2.46 (s, 3H), 2.38-2.19 (m, 1H), 2.13-1.99 (m, 1H), 1.98-1.84 (m, 1H), 1.69-1.30 (m, 4H), 0.95 (d, J=6.0 Hz, 2H), 0.71 (d, J=6.2 Hz, 2H). ¹³C NMR (151 MHz, DMSO-d6) δ 171.56, 168.30, 168.09, 167.91, 167.47, 159.30, 158.29 (d, J=239.9 Hz), 155.87, 153.27 (d, J=245.5 Hz), 151.81, 151.45, 149.47, 148.82, 147.93, 146.33, 142.19, 138.42-137.45 (m), 135.64 (d, J=11.8 Hz), 135.21, 131.57, 131.37, 131.26, 130.98, 127.90, 127.66, 127.20, 123.82, 123.59, 123.01, 122.44 (d, J=7.9 Hz), 121.08, 117.44-116.65 (m), 115.05 (d, J=22.1 Hz), 114.55, 112.16, 108.98 (d, J=22.3 Hz), 108.64, 101.96, 99.03. LC-MS (ESI); m/z: [M+H]⁺ Calcd. for C₆₂H₆₄F₂N₇O₁₂, 1168.4301. Found 1168.4722.

### Example 47

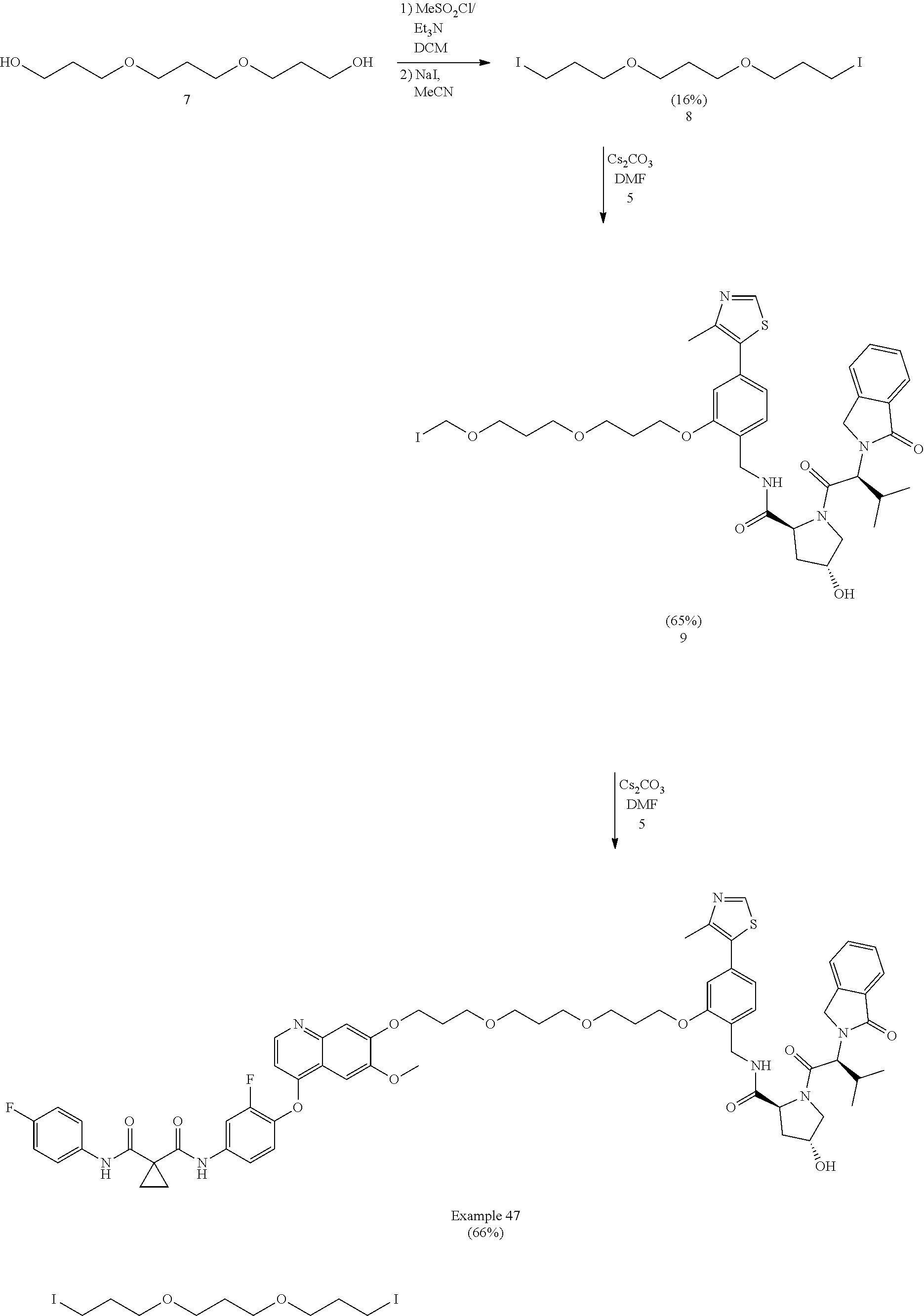 [SMILES: OCCCOCCCOCCCO ; ICCCOCCCOCCCI ; Cc1ncsc1-c1ccc(CNC(=O)[C@@H]2C[C@@H](O)CN2C(=O)[C@H](C(C)C)N2Cc3ccccc3C2=O)c(OCCCOCCCOCI)c1 ; COc1cc2c(Oc3ccc(NC(=O)C4(C(=O)Nc5ccc(F)cc5)CC4)cc3F)ccnc2cc1OCCCOCCCOCCCOc1cc(-c2scnc2C)ccc1CNC(=O)[C@@H]1C[C@@H](O)CN1C(=O)[C@H](C(C)C)N1Cc2ccccc2C1=O ; ICCCOCCCOCCCI]

### 1,3-bis(3-iodopropoxy)propane (8)

To a solution of 3-[3-(3-hydroxypropoxy)propoxy]propan-1-ol (334 mg, 1.74 mmol) in Dichloromethane (30 ml) was added TEA (0.73 ml, 5.21 mmol), then reaction mixture was cooled to −10° C. (water ice/acetone bath) and Mesyl chloride (0.15 ml, 1.91 mmol) was added dropwise. The reaction mixture was stirred for 1 h at the same temperature. By TLC no starting material (Hex:AcOEt, 3:7), and a less polar compound was formed. Reaction mixture was poured into an aqueous solution of NaHCO₃ (5 mL) and product extracted with DCM (5 mL, 2×), the organic extracts were combined, dried (Na₂SO₄), and evaporated under vacuum. The crude product (469 mg, quantitative yield) was used in the next step. The crude product from above (mixture of mesylates) was used without any further purification. Thus, 3-[3-(3-hydroxypropoxy)propoxy]propyl methanesulfonate (469 mg, 1.73 mmol) was diluted in Acetonitrile (100 ml) and NaI (1300.22 mg, 8.67 mmol) was added, the reaction mixture was stirred at room temperature for 12 h (overnight) and then at reflux temperature for 4 h. The reaction was filtered through a Celite pad under vacuum, and the filtrate was evaporated to dryness. The residue was dissolved in AcOEt (50 mL) and water (50 mL), the organic layer was separated, dried (Na₂SO₄) and evaporated under vacuum. Crude product was purified by flash chromatography (SiO₂-40 g, grad. Hex:AcOEt, 2 to 60% AcOEt in 20 min), to give 114 mg of product as an oil (16% yield) of a less polar product (bis-iodide) and 155 mg (30% yield) of a more polar product (mono-iodide): ¹H NMR (400 MHz, Chloroform-d) δ 3.49 (dtd, J=12.7, 6.0, 1.1 Hz, 8H), 3.28 (t, J=7.1 Hz, 4H), 2.05 (p, J=6.7 Hz, 4H), 1.95-1.67 (m, 2H). ¹³C NMR (101 MHz, cdcl3) δ 70.21, 67.96, 33.58, 30.17, 3.70. HRMS (ESI); m/z [M+H]⁺: Calcd. for C₉H₁₉I₂O₂, 412.9474. Found 412.9474. More polar product (mono-iodide): ¹H NMR (400 MHz, Chloroform-d) δ 3.77 (q, J=4.8 Hz, 1H), 3.62 (t, J=5.7 Hz, 2H), 3.51 (q, J=6.7 Hz, 4H), 3.47 (t, J=5.9 Hz, 2H), 3.27 (t, J=6.8 Hz, 2H), 2.43 (bs, 1H), 2.04 (p, J=6.3 Hz, 2H), 1.93-1.72 (m, 4H). ¹³C NMR (101 MHz, cdcl3) δ 70.62, 70.23, 68.42, 68.04, 62.44, 33.49, 32.11, 30.12, 3.67. LC-MS (ESI); m/z [M+H]⁺: Calcd. for C₉H₂₀IO₃.

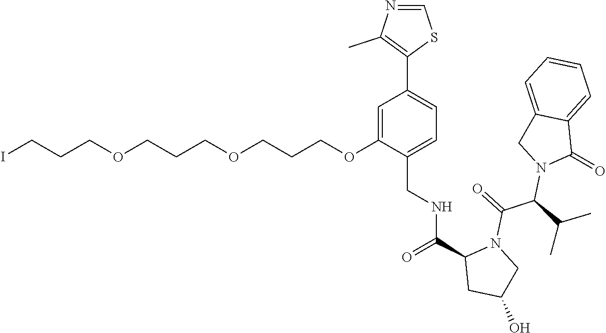 [SMILES: Cc1ncsc1-c1ccc(CNC(=O)[C@@H]2C[C@@H](O)CN2C(=O)[C@H](C(C)C)N2Cc3ccccc3C2=O)c(OCCCOCCCOCCCI)c1]

### (2S,4R)-4-hydroxy-N-(2-(3-(3-(3-iodopropoxy)propoxy)propoxy)-4-(4-methylthiazol-5-yl)benzyl)-1-((S)-3-methyl-2-(1-oxoisoindolin-2-yl)butanoyl)pyrrolidine-2-carboxamide (9)

To a mixture of (2) (30 mg, 0.05 mmol) and 1,3-bis(3-iodopropoxy)propane (112.65 mg, 0.27 mmol) in N,N-Dimethylforma-mide (1 mL) was added Cs₂CO₃ (53.45 mg, 0.16 mmol). After stirring at room temperature for 4 hrs, the reaction mixture was diluted with AcOEt (10 mL) and washed with water (5×10 mL), organic phase was dried (Na₂SO₄, and evaporated under vacuum. Crude product was purified over a short column of SiO₂ (Gradient, DCM 100% to DCM:MeOH:NH₄OH, 90:9:1) to give 30 mg of product (65% yield). ¹H NMR (400 MHz, DMSO-d6) δ 8.99 (s, 1H), 8.38 (t, J=5.9 Hz, 1H), 7.71 (d, J=7.5 Hz, 1H), 7.68-7.55 (m, 2H), 7.55-7.44 (m, 1H), 7.34 (d, J=7.6 Hz, 1H), 7.12-6.91 (m, 2H), 5.10 (d, J=4.1 Hz, 1H), 4.71 (d, J=10.9 Hz, 1H), 4.53 (dd, 2H), 4.44-4.17 (m, 4H), 4.11 (t, J=6.1 Hz, 2H), 3.78 (dd, J=10.5, 4.3 Hz, 1H), 3.69 (d, J=10.7 Hz, 1H), 3.56 (t, J=6.2 Hz, 2H), 3.44 (t, J=6.3 Hz, 2H), 3.40 (t, J=6.4 Hz, 2H), 3.34 (t, 2H), 3.24 (t, J=6.7 Hz, 2H), 2.47 (s, 3H), 2.38-2.25 (m, 1H), 2.22-1.82 (m, 6H), 1.72 (p, J=6.3 Hz, 2H), 0.97 (d, J=6.5 Hz, 3H), 0.74 (d, J=6.6 Hz, 3H). ¹³C 13C NMR (151 MHz, dmso) δ 171.49, 168.08, 167.46, 155.87, 151.46, 147.88, 142.19, 131.57, 131.37, 131.28, 131.00, 127.90, 127.79, 127.00, 123.61, 123.01, 120.84, 111.66, 69.41, 68.61, 67.06, 67.02, 66.58, 64.82, 58.68, 57.77, 55.42, 46.81, 38.09, 37.06, 32.83, 29.57, 29.10, 28.39, 18.89, 18.63, 16.04, 16.04. LC-MS (ESI); m/z: [M+H]⁺ Calcd. for C₃₈H₅₀IN₄O₇S, 833.2444. Found 833.2540.

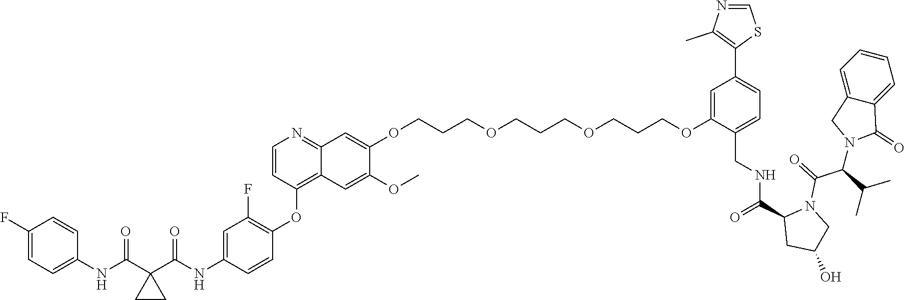 [SMILES: COc1cc2c(Oc3ccc(NC(=O)C4(C(=O)Nc5ccc(F)cc5)CC4)cc3F)ccnc2cc1OCCCOCCCOCCCOc1cc(-c2scnc2C)ccc1CNC(=O)[C@@H]1C[C@@H](O)CN1C(=O)[C@H](C(C)C)N1Cc2ccccc2C1=O]

### N-(3-fluoro-4-((7-(3-(3-(3-(2-(((2S,4R)-4-hydroxy-1-((S)-3-methyl-2-(1-oxoisoindolin-2-yl)butano-yl)pyrrolidine-2-carboxamido)methyl)-5-(4-methylthiazol-5-yl)phenoxy)propoxy)-propoxy)propoxy)-6-methoxyquinolin-4-yl)oxy)phenyl)-N-(4-fluorophenyl)cyclopropane-1,1-dicarboxamide (PROTAC 47)

To a mixture of (9) (15 mg, 0.02 mmol) and (2) (9.1 mg, 0.02 mmol) in N,N-Dimethylformamide (1 mL) was added Cs₂CO₃ (11.74 mg, 0.04 mmol). After stirring at room temperature for 12 hrs (overnight), the reaction mixture was diluted with AcOEt (10 mL) and washed with brine (5×10 mL), organic phase was dried (Na₂SO₄, and evaporated under vacuum. Crude product was purified by PTLC (DCM:MeOH, 9:1) to give 14.5 mg of product (66% yield). ¹H NMR (400 MHz, DMSO-d6) δ 10.40 (s, 1H), 10.02 (s, 1H), 8.97 (s, 1H), 8.45 (d, J=5.2 Hz, 1H), 8.39 (t, J=5.5 Hz, 1H), 7.90 (d, J=13.2 Hz, 1H), 7.78-7.28 (m, 10H), 7.16 (t, J=8.6 Hz, 2H), 7.05-6.89 (m, 2H), 6.40 (d, J=5.1 Hz, 1H), 5.11 (d, J=3.9 Hz, 1H), 4.71 (d, J=10.8 Hz, 1H), 4.64-4.21 (m, 6H), 4.18 (t, J=6.0 Hz, 2H), 4.08 (t, J=5.8 Hz, 2H), 3.93 (s, 3H), 3.82-3.66 (m, 2H), 3.48 (dt, J=33.6, 5.4 Hz, 8H), 2.46 (s, 3H), 2.31 (dd, J=17.1, 6.3 Hz, 1H), 2.18-1.81 (m, 6H), 1.74 (p, J=6.0 Hz, 2H), 1.63-1.34 (m, 4H), 0.96 (d, J=6.3 Hz, 3H), 0.72 (d, J=6.4 Hz, 3H). ¹³C NMR (151 MHz, DMSO-d6) δ 171.51, 168.29, 168.09, 167.91, 167.47, 159.30, 158.29 (d, J=240.2 Hz), 155.88, 153.26 (d, J=245.2 Hz), 151.91, 151.42, 149.55, 148.80, 147.87, 146.36, 142.19, 138.01 (d, J=9.8 Hz), 135.66 (d, J=12.3 Hz), 135.20 (d, J=2.5 Hz), 131.57, 131.37, 131.28, 131.01, 127.85 (d, J=15.1 Hz), 127.01, 123.81, 123.60, 123.01, 122.45 (d, J=7.9 Hz), 120.84, 116.91, 115.11, 114.97, 114.47, 111.64, 108.97 (d, J=22.9 Hz), 108.48, 101.93, 99.01, 68.63, 67.09, 67.08, 66.56, 66.55, 65.42, 64.79, 58.70, 57.78, 55.77, 55.44, 46.81, 38.10, 37.06, 31.91, 29.61, 29.06, 28.89, 28.40, 18.87, 18.62, 16.00, 15.35. LC-MS (ESI); m/z: [M+H]⁺ Calcd. for C₆₅H₇₀F₂N₇O₁₂S, 1210.4771. Found 1210.5192.

### Example 48

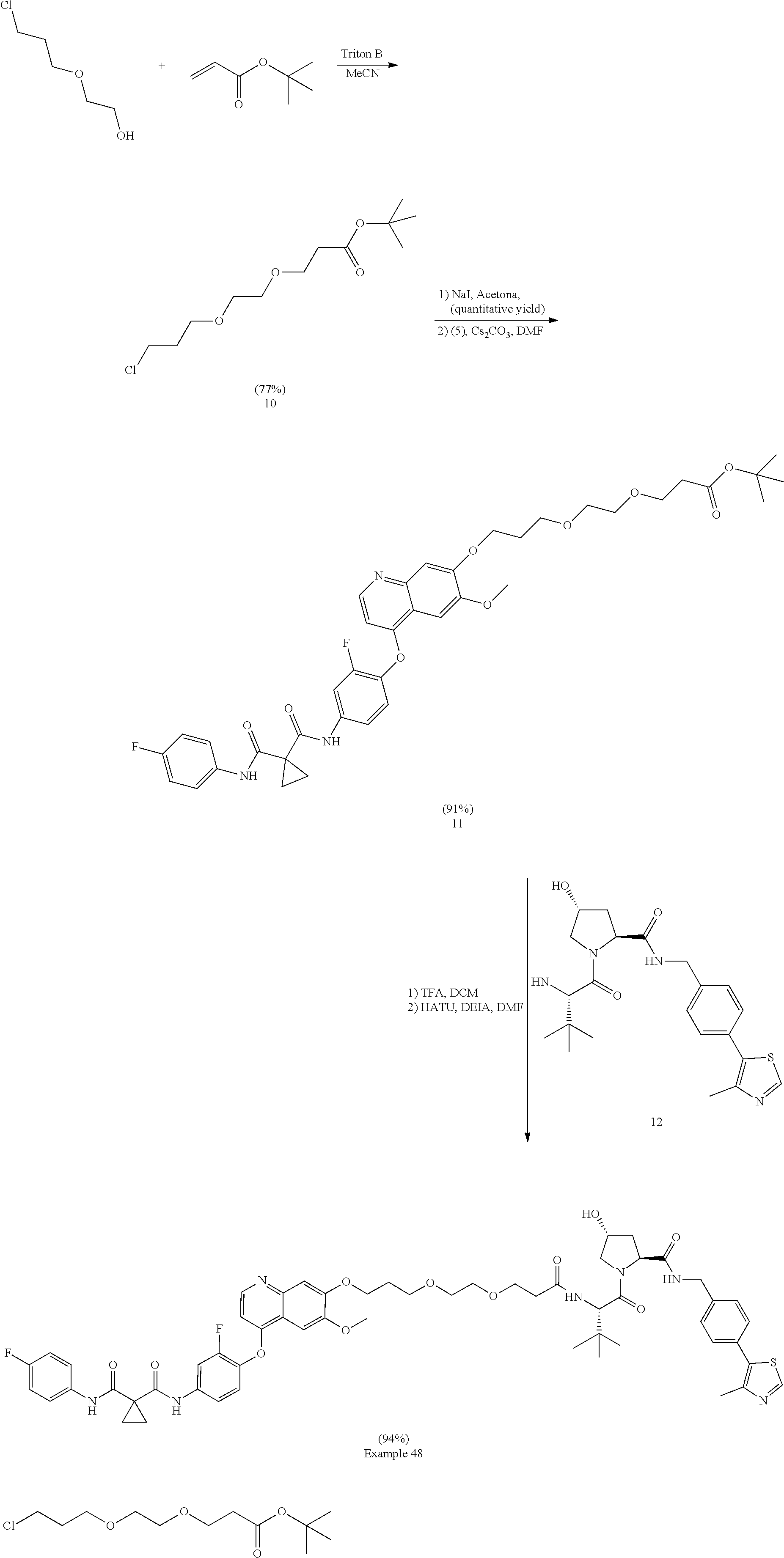

### tert-butyl 3-(2-(3-chloropropoxy)ethoxy)propanoate (10)

To a solution of 2-(3-chloropropoxy)ethan-1-ol (1.48 g, 10.68 mmol) in acetonitrile (20 mL) was added tert-butyl prop-2-enoate (7.75 ml, 128.17 mmol) followed by Triton B (447 mg, 1.06 mmol, in 40% by weight in water). The mixture was stirred at room temperature for 12 hours (overnight). The mixture was concentrated in vacuum and crude product was purified by CC (SiO₂-80 g, gradient Hex:AcOEt, 98:2 to 8:2) to give 2.21 g of product as an oil (77% yield). ¹H NMR (500 MHz, Chloroform-d) δ 3.71 (t, J=6.6 Hz, 2H), 3.63 (t, J=6.4 Hz, 2H), 3.62-3.54 (m, 8H), 2.50 (t, J=6.6 Hz, 2H), 2.02 (p, J=6.2 Hz, 2H), 1.44 (s, 9H). ¹³C NMR (126 MHz, cdcl3) δ 171.02, 80.66, 77.36, 70.46, 67.79, 67.08, 42.09, 36.44, 32.86, 28.25. LC-MS (ESI); m/z: [M+H]⁺ Calcd. for C₁₂H₂₃ClO₄Na, 289.1182. Found 289.1364.

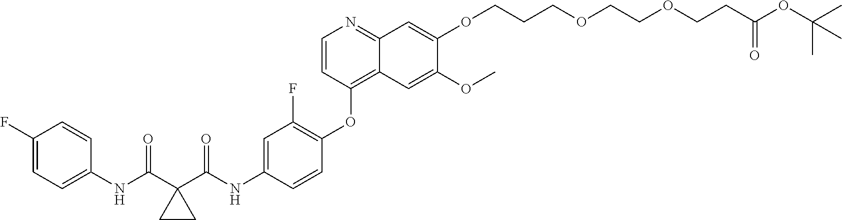 [SMILES: COc1cc2c(Oc3ccc(NC(=O)C4(C(=O)Nc5ccc(F)cc5)CC4)cc3F)ccnc2cc1OCCCOCCOCCC(=O)OC(C)(C)C]

### tert-butyl 3-(2-(3-((4-(2-fluoro-4-(1-((4-fluorophenyl)carbamoyl)cyclopropane-1-carboxamido)-phenoxy)-6-methoxyquinolin-7-yl)oxy)propoxy)ethoxy)propanoate (11)

To a solution of (10) (188 mg, 0.70 mmol) in Acetone (15 ml) was added NaI (528.19 mg, 3.52 mmol). The reaction mixture was stirred at reflux temperature for 24 h, then the solvent was removed under vacuum and crude product was dissolved in EtOAc (15 mL) and an aqueous solution of Na₂SO₃ (10%, 10 mL), organic layer was separated, washed with water (10 mL), dried (Na₂SO₄) and evaporated under vacuum. Crude product was pure by NMR (>95% purity, 245 mg, 97% yield), it was used in the next step without any further purification; ¹H NMR (500 MHz, Chloroform-d) δ 3.71 (t, J=6.6 Hz, 2H), 3.64-3.55 (m, 4H), 3.52 (t, J=5.9 Hz, 2H), 3.27 (t, J=6.8 Hz, 2H), 2.50 (t, J=6.6 Hz, 2H), 2.05 (p, J=6.3 Hz, 2H), 1.44 (s, 9H). ¹³C 13C NMR (126 MHz, cdcl3) δ 171.01, 80.66, 70.68, 70.45, 70.44, 67.06, 36.42, 33.54, 28.25, 3.57. To a mixture of (5) (13.3 mg, 0.03 mmol) and tert-butyl 3-[2-(3-iodopropoxy)ethoxy]propanoate from above (18.85 mg, 0.05 mmol) in N,N-Dimethylformamide (1 mL) was added Cs₂CO₃ (17.15 mg, 0.05 mmol). After stirring at room temperature for 12 hrs (overnight), the reaction mixture was diluted with AcOEt (10 mL) and washed with water (5×5 mL), organic phase was evaporated under vacuum. Crude product was purified by PTLC (DCM:MeOH, 9:1) to give 17 mg of product (91% yield). ¹H NMR (500 MHz, DMSO-d6) δ 10.39 (s, 1H), 10.01 (s, 1H), 8.45 (d, J=5.0 Hz, 1H), 7.89 (d, J=13.1 Hz, 1H), 7.73-7.58 (m, 2H), 7.55-7.46 (m, 2H), 7.46-7.31 (m, 2H), 7.14 (t, J=8.7 Hz, 2H), 6.40 (d, J=4.3 Hz, 1H), 4.19 (t, J=4.9 Hz, 2H), 3.94 (s, 3H), 3.63-3.52 (m, 4H), 3.53-3.42 (m, 4H), 2.38 (t, J=6.1 Hz, 2H), 2.10-1.94 (m, 2H), 1.56-1.40 (m, 4H), 1.35 (s, 9H). ¹³C NMR (151 MHz, DMSO-d6) δ 170.80, 168.70, 168.32, 159.72, 158.71 (d, J=240.1 Hz), 153.68 (d, J=245.1 Hz), 152.30, 149.97, 149.22, 146.79, 138.42 (d, J=9.8 Hz), 136.09 (d, J=12.4 Hz), 135.60, 122.86 (d, J=7.9 Hz), 117.32 (d, J=3.2 Hz), 115.52, 115.37, 114.90, 109.40 (d, J=22.8 Hz), 108.93, 102.36, 99.43, 80.08, 70.07, 70.00, 67.28, 66.68, 65.84, 56.19, 36.26, 32.30, 29.35, 28.13, 15.77. LC-MS (ESI); m/z: [M+H]⁺ Calcd. for C₃₉H₄₄F₂N₃O₉, 736.3045. Found 736.3098.

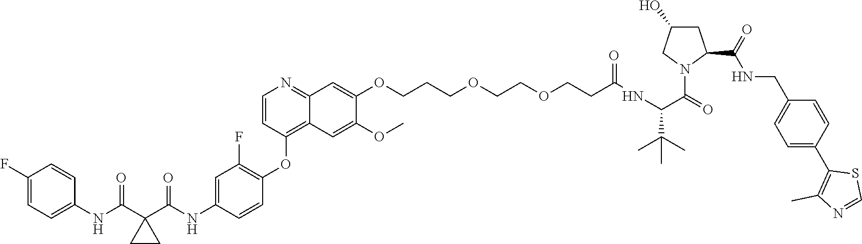 [SMILES: COc1cc2c(Oc3ccc(NC(=O)C4(C(=O)Nc5ccc(F)cc5)CC4)cc3F)ccnc2cc1OCCCOCCOCCC(=O)N[C@H](C(=O)N1C[C@H](O)C[C@H]1C(=O)NCc1ccc(-c2scnc2C)cc1)C(C)(C)C]

### N-(3-fluoro-4-((7-(3-(2-(3-(((S)-1-((2S,4R)-4-hydroxy-2-((4-(4-methylthiazol-5-yl)benzyl) carbamoyl)pyrrolidin-1-yl)-3,3-dimethyl-1-oxobutan-2-yl)amino)-3-oxopropoxy)ethoxy)-propoxy)-6-methoxyquinolin-4-yl)oxy)phenyl)-N-(4-fluorophenyl)cyclopropane-1,1-dicarboxa-mide (PRPTAC 48)

A solution of tert-butyl ester (11) (10.3 mg, 0.01 mmol) in a mixture of TFA (1 ml) and Dichloromethane (2 ml) was stirred for 1 h. Then the solvent was removed under vacuum and crude product was dried under high vacuum for 2 h. Crude product was used in the next step without any further purification (9.5 mg, quantitative yield). LC-MS (ESI); m/z: [M+H]⁺ Calcd. for C₃₅H₃₆F₂N₃O₉, 680.2419. Found 680.2421.

To a solution of the product from above (8.79 mg, 0.01 mmol) and (2S,4R)-1-[(2S)-2-amino-3,3-dimethyl-butanoyl]-4-hydroxy-N-[[4-(4-methylthiazol-5-yl)phenyl]methyl]-pyrrolidine-2-carboxa-mide; hydrochloride (7.25 mg, 0.02 mmol)) in N,N-Dimethylformamide (2 ml) was added N,N-Diisopropylethylamine (0.17 ml, 0.99 mmol) and O-(7-Azabenzotriazol-1-yl)-N,N,N′,N′-tetramethyluronium hexafluorophosphate (7.38 mg, 0.02 mmol) at room temperature. The reaction mixture was stirred for 12 h (overnight) at the same temperature. TLC (DCM:MeOH:NH₄OH, 90:9:1) shows no starting materials. Reaction mixture was diluted with ACOEt (10 mL), washed with water (4×10 mL), dried (Na₂SO₄) and evaporated under vacuum. Crude product was purified by PTLC (DCM:MeOH:NH₄OH, 90:9:1), to give 14.5 mg of product (94% yield). ¹H NMR (500 MHz, DMSO-d6) δ 10.39 (s, 1H), 10.01 (s, 1H), 8.96 (s, 1H), 8.58 (t, J=5.7 Hz, 1H), 8.47 (d, J=5.1 Hz, 1H), 7.91 (t, J=10.0 Hz, 2H), 7.64 (d, J=13.2 Hz, 2H), 7.52 (d, J=6.2 Hz, 2H), 7.49-7.25 (m, 5H), 7.15 (t, J=8.5 Hz, 2H), 6.41 (d, J=5.1 Hz, 1H), 5.14 (d, J=3.4 Hz, 1H), 4.55 (d, J=9.3 Hz, 1H), 4.53-4.28 (m, 3H), 4.21 (dt, J=11.3, 5.6 Hz, 4H), 3.94 (s, 3H), 3.78-3.41 (m, 9H), 2.61-2.50 (m, 1H), 2.43 (s, 3H), 2.35 (dt, J=13.9, 5.8 Hz, 1H), 2.12-1.97 (m, 3H), 1.95-1.84 (m, 1H), 1.48 (s, 4H), 0.92 (s, 9H). ¹³C NMR (151 MHz, DMSO-d6) δ 172.37, 170.39, 169.96, 168.72, 168.33, 159.79, 158.71 (d, J=240.2 Hz), 153.67 (d, J=245.3 Hz), 152.34, 151.84, 149.99, 149.17, 148.12, 146.67, 139.90, 138.43 (d, J=9.7 Hz), 136.07 (d, J=12.4 Hz), 135.60 (d, J=2.6 Hz), 131.58, 130.05, 129.05, 127.83, 124.21, 122.87 (d, J=7.8 Hz), 117.33, 115.45 (d, J=22.2 Hz), 114.90, 109.40 (d, J=23.0 Hz), 108.84, 102.37, 99.45, 70.01, 69.92, 69.31, 67.39, 67.33, 65.87, 59.14, 56.81, 56.72, 56.21, 42.08, 38.37, 36.10, 35.78, 32.32, 29.32, 26.73, 16.36, 15.77. LC-MS (ESI); m/z [M+H]⁺: Calcd. for C₅₇H₆₄F₂N₇O₁₁S, 1092.4352. Found 1092.4733.

### Example 49

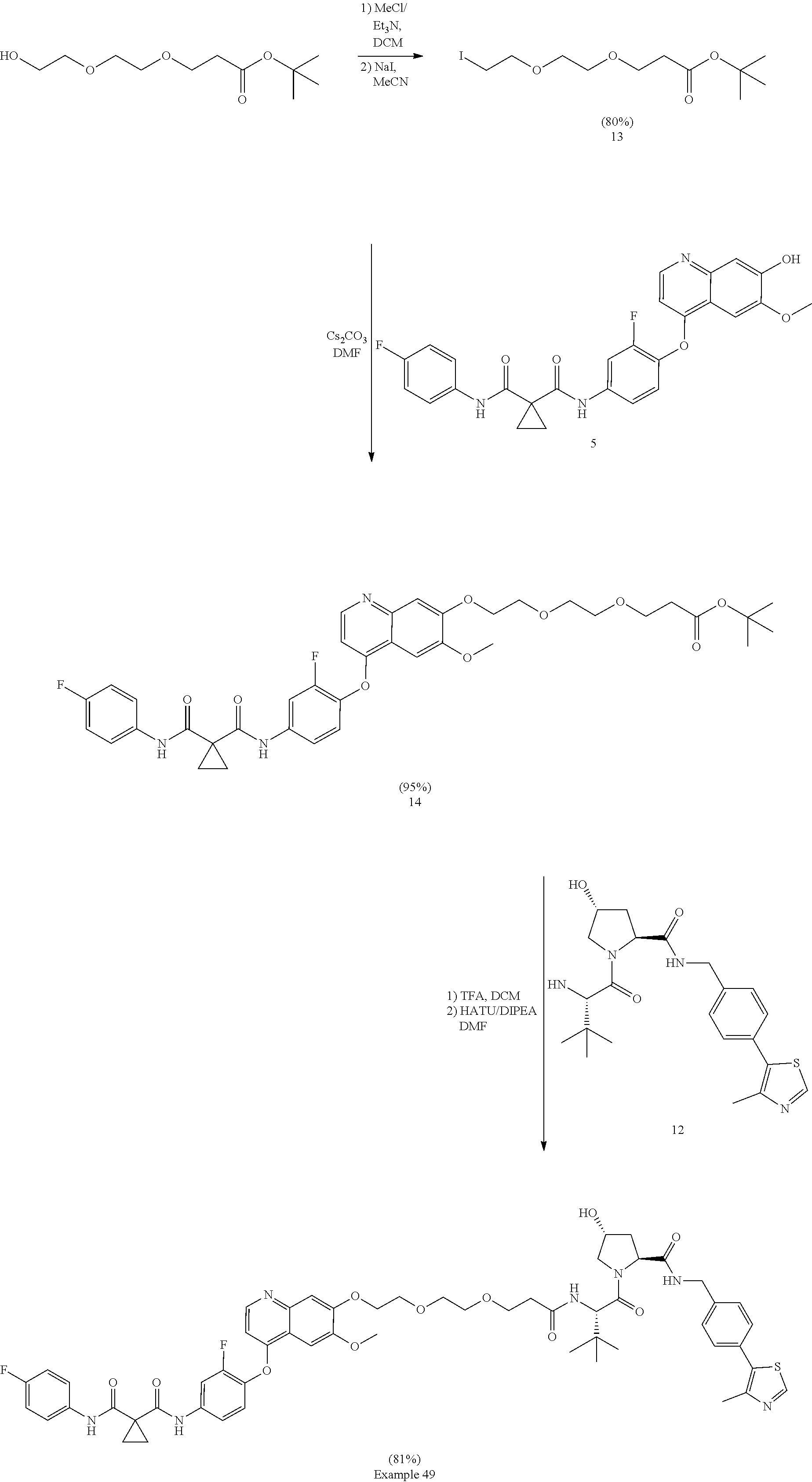 [SMILES: CC(C)(C)OC(=O)CCOCCOCCO ; CC(C)(C)OC(=O)CCOCCOCCI ; COc1cc2c(Oc3ccc(NC(=O)C4(C(=O)Nc5ccc(F)cc5)CC4)cc3F)ccnc2cc1O ; COc1cc2c(Oc3ccc(NC(=O)C4(C(=O)Nc5ccc(F)cc5)CC4)cc3F)ccnc2cc1OCCOCCOCCC(=O)OC(C)(C)C ; Cc1ncsc1-c1ccc(CNC(=O)[C@@H]2C[C@@H](O)CN2C(=O)[C@@H](N)C(C)(C)C)cc1 ; COc1cc2c(Oc3ccc(NC(=O)C4(C(=O)Nc5ccc(F)cc5)CC4)cc3F)ccnc2cc1OCCOCCOCCC(=O)N[C@H](C(=O)N1C[C@H](O)C[C@H]1C(=O)NCc1ccc(-c2scnc2C)cc1)C(C)(C)C]

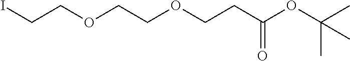 [SMILES: CC(C)(C)OC(=O)CCOCCOCCI]

### tert-Butyl 3-(2-(2-iodoethoxy)ethoxy)propanoate (13)

To a solution of tert-butyl 3-[2-(2-hydroxyethoxy)ethoxy]propanoate (350 mg, 1.49 mmol) in DCM (15 ml) was added TEA (0.62 ml, 4.48 mmol), then the reaction mixture was cooled to 0° C. (water ice/acetone bath) and Mesyl chloride (0.14 ml, 1.79 mmol) was added dropwise. The reaction mixture was stirred for 1 h at room temperature. By TLC no starting material (Hex:AcOEt, 3:7). Reaction mixture was poured into an aqueous solution of NaHCO₃ (20 mL) and product extracted with DCM (20 mL, 2×), the organic extracts were combined, dried (Na₂SO₄), and evaporated under vacuum. The crude product (mesylate) was used in the next step without any further purification (>95% pure by NMR): ¹H NMR (500 MHz, Chloroform-d) δ 4.44-4.28 (m, 2H), 3.76 (dd, J=5.2, 3.8 Hz, 2H), 3.70 (t, J=6.4 Hz, 2H), 3.68-3.57 (m, 4H), 3.07 (s, 3H), 2.49 (t, J=6.4 Hz, 2H), 1.45 (s, 9H).

Crude mixture from previous step was diluted in Acetonitrile (5 ml) and NaI (335.88 mg, 2.24 mmol) was added, the reaction mixture was stirred at 70° C. for 12 h (overnight). By TLC no starting material (Hex:AcOEt, 7:3), the reaction was poured into an aqueous solution of Na₂S₂O₃ (10%, 50 mL) and product was extracted with DCM (2×50 mL). Organic extracts were combined, dried (Na₂SO₄) and evaporated under vacuum. Crude product was purified by flash chromatography (SiO₂-40 g, grad. Hex:AcOEt, 2 to 40% in 15 min), to give 413 mg of product as an oil (80% yield). ¹H NMR (500 MHz, Chloroform-d) δ 3.73 (dt, J=13.5, 6.6 Hz, 4H), 3.68-3.54 (m, 4H), 3.24 (t, J=6.9 Hz, 2H), 2.50 (t, J=6.5 Hz, 2H), 1.44 (s, 9H). ¹³C NMR (151 MHz, cdcl3) δ 171.00, 80.69, 72.11, 70.50, 70.27, 67.09, 36.39, 28.25, 3.02. LC-MS (ESI); m/z: [M+Na]⁺ Calcd. for C₁₁H₂₁IO₄Na, 367.0382. Found 367.0943.

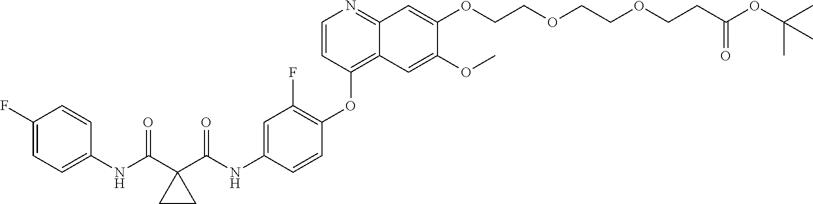 [SMILES: COc1cc2c(Oc3ccc(NC(=O)C4(C(=O)Nc5ccc(F)cc5)CC4)cc3F)ccnc2cc1OCCOCCOCCC(=O)OC(C)(C)C]

### tert-Butyl 3-(2-(2-((4-(2-fluoro-4-(1-((4-fluorophenyl)carbamoyl)cyclopropane-1-carboxamido)-phenoxy)-6-methoxyquinolin-7-yl)oxy)ethoxy)ethoxy)propanoate (14)

To a mixture of the tert-butyl ester (13) (10.3 mg, 0.02 mmol) and tert-butyl 3-[2-(2-iodoethoxy)ethoxy]propanoate (14.03 mg, 0.04 mmol) in N,N-Dimethylformamide (1 mL) was added Cs₂CO₃ (13.28 mg, 0.04 mmol). After stirring at room temperature for 12 hrs (overnight), the reaction mixture was diluted with AcOEt (10 mL) and washed with water (5×5 mL), organic phase was evaporated under vacuum. Crude product was purified by PTLC (DCM:MeOH, 9:1) to give 14 mg of product (95% yield).

¹H NMR (400 MHz, DMSO-d6) δ 10.39 (s, 1H), 10.01 (s, 1H), 8.48 (d, J=5.2 Hz, 1H), 7.90 (d, J=13.2 Hz, 1H), 7.70-7.59 (m, 2H), 7.59-7.47 (m, 2H), 7.48-7.34 (m, 2H), 7.15 (t, J=8.8 Hz, 2H), 6.43 (d, J=5.1 Hz, 1H), 4.27 (t, J=9.2 Hz, 1H), 3.96 (s, 3H), 3.84 (t, J=4.2 Hz, 2H), 3.70-3.48 (m, 6H), 2.42 (t, J=6.2 Hz, 2H), 1.48 (s, 4H), 1.38 (d, J=1.6 Hz, 9H). ¹³C NMR (151 MHz, DMSO-d6) δ 170.39, 168.29, 167.91, 159.32, 158.30 (d, J=240.2 Hz), 153.27 (d, J=245.6 Hz), 151.81, 149.48, 148.78, 146.29, 138.01 (d, J=9.8 Hz), 135.66 (d, J=12.5 Hz), 135.17 (d, J=2.5 Hz), 123.78, 122.44 (d, J=7.8 Hz), 116.90 (d, J=3.2 Hz), 114.94, 114.57, 108.99 (d, J=23.0 Hz), 108.56, 101.96, 99.05, 79.70, 69.90, 68.69, 68.03, 66.26, 55.73, 35.83, 31.86, 27.74, 15.37. LC-MS (ESI); m/z: [M+H]⁺ Calcd. for C₃₈H₄₂F₂N₃O₉, 722.2889. Found 722.3494.

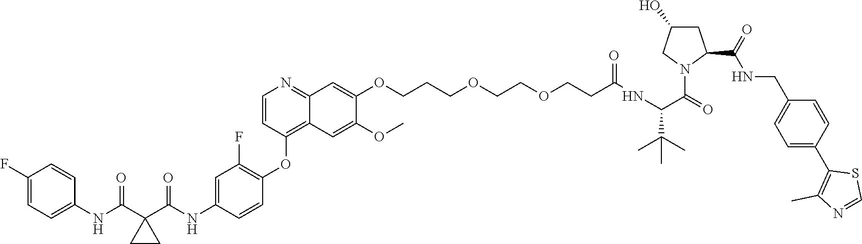 [SMILES: COc1cc2c(Oc3ccc(NC(=O)C4(C(=O)Nc5ccc(F)cc5)CC4)cc3F)ccnc2cc1OCCCOCCOCCC(=O)N[C@H](C(=O)N1C[C@H](O)C[C@H]1C(=O)NCc1ccc(-c2scnc2C)cc1)C(C)(C)C]

### N-(3-fluoro-4-((7-(2-(2-(3-(((S)-1-((2S,4R)-4-hydroxy-2-((4-(4-methylthiazol-5-yl)benzyl)carba-moyl)pyrrolidin-1-yl)-3,3-dimethyl-1-oxobutan-2-yl)amino)-3-oxopropoxy)ethoxy)ethoxy)-6-methoxyquinolin-4-yl)oxy)phenyl)-N-(4-fluorophenyl)cyclopropane-1,1-dicarboxamide (PROTAC 49)

A solution of tert-butyl (14) (13.5 mg, 0.02 mmol) in a mixture of TFA (0.7 ml, 9.42 mmol) and Dichloromethane (2 ml) was stirred for 1 h. Then the solvent was removed under vacuum and crude product was dried under high vacuum for 2 h. Crude product was used in the next step without any further purification (12.4 mg, quantitative yield). HRMS (ESI); m/z: [M+H]⁺ Calcd. for C₃₄H₃₄F₂N₃O₉, 666.2263. Found 666.2394.

To a solution of 3-[2-[2-[[4-[2-fluoro-4-[[1-[(4-fluorophenyl)carbamoyl]cyclopropane-carbonyl]amino]phenoxy]-6-methoxy-7-quinolyl]oxy]ethoxy]ethoxy]propanoic acid from above (12.45 mg, 0.02 mmol) and (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide; hydrochloride (12) (10.48 mg, 0.02 mmol) in N,N-Dimethylformamide (2 ml) was added N,N-Diisopropylethylamine (0.25 ml, 1.41 mmol) and 0-(7-Azabenzotriazol-1-yl)-N,N,N′,N′-tetramethyluronium hexafluorophosphate (10.67 mg, 0.03 mmol) at room temperature. The reaction mixture was stirred for 12 h (overnight) at the same temperature. TLC (DCM:MB, 1:1) shows no starting materials. Reaction mixture was diluted with ACOEt (10 mL), washed with water (4×10 mL), dried (Na₂SO₄) and evaporated under vacuum. Crude product was purified by PTLC (DCM:MeOH:NH₄OH, 90:9:1), to give 16.5 mg of product (81% yield). ¹H NMR (500 MHz, DMSO-d6) δ 10.39 (s, 1H), 10.01 (s, 1H), 8.97 (s, 1H), 8.61 (t, J=5.9 Hz, 1H), 8.47 (d, J=5.2 Hz, 1H), 8.03-7.84 (m, 2H), 7.64 (dd, J=8.6, 5.2 Hz, 2H), 7.55-7.48 (m, 2H), 7.47-7.30 (m, 6H), 7.15 (t, J=8.8 Hz, 2H), 6.42 (d, J=5.2 Hz, 1H), 5.15 (d, J=3.2 Hz, 1H), 4.56 (d, J=9.4 Hz, 1H), 4.49-4.39 (m, 2H), 4.38-4.33 (m, 1H), 4.31-4.17 (m, 3H), 3.95 (s, 3H), 3.84 (t, J=4.1 Hz, 2H), 3.73-3.48 (m, 8H), 2.60-2.52 (m, 1H), 2.43 (s, 3H), 2.38 (dt, J=14.3, 6.0 Hz, 1H), 2.10-2.00 (m, 1H), 1.95-1.87 (m, 1H), 1.61-1.40 (m, 4H), 0.93 (s, 9H). ¹³C NMR (151 MHz, DMSO-d6) δ 171.99, 170.02, 169.57, 168.32, 167.94, 159.37, 158.32 (d, J=240.2 Hz), 153.29 (d, J=245.2 Hz), 151.85, 151.46, 149.51, 148.81, 147.73, 146.28, 139.51, 138.04 (d, J=9.9 Hz), 135.67 (d, J=12.4 Hz), 135.21 (d, J=2.6 Hz), 131.19, 129.65, 128.65, 127.45, 123.84, 122.48 (d, J=7.9 Hz), 116.95, 115.14, 114.99, 109.01 (d, J=22.8 Hz), 108.57, 102.00, 99.07, 69.93, 69.54, 68.92, 68.73, 68.08, 67.00, 58.76, 56.35, 55.77, 41.69, 37.97, 35.70, 35.40, 31.93, 26.34, 15.96, 15.37. LC-MS (ESI); m/z [M+H]⁺: Calcd. for C₅₆H₆₂F₂N₇O₁₁S, 1078.4196. Found 1078.4576.

### Example 50

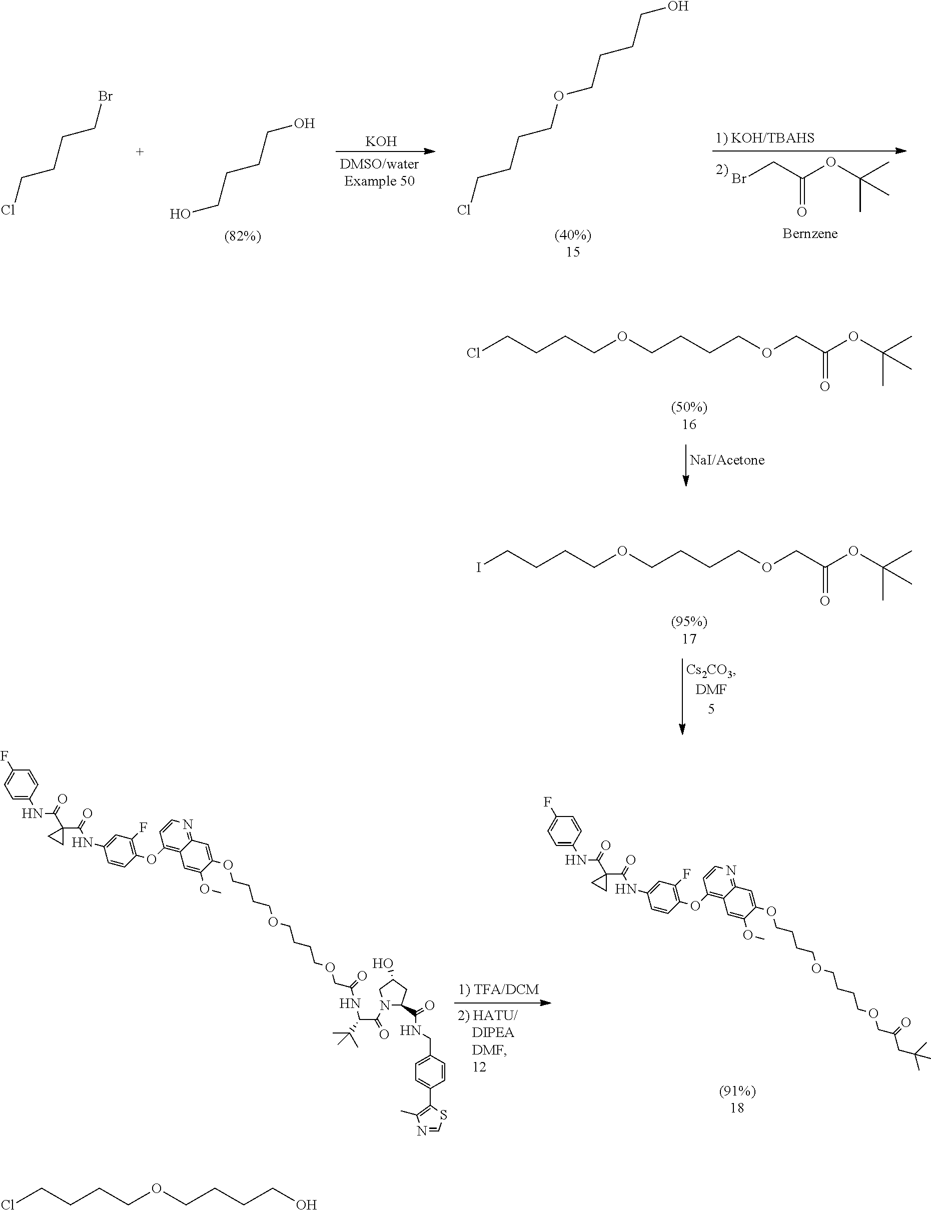

### 4-(4-Chlorobutoxy)butan-1-ol (15)

A solution of butane-1,4-diol (23.14 ml, 260.35 mmol) and KOH (2.43 g, 43.39 mmol) in a mixture of DMSO (135 ml) and distilled water (15 mL) was stirred for 15 min at rt. Then the mixture was cooled down to 0° C. and 1-bromo-4-chloro-butane (5 ml, 43.39 mmol) was added dropwise over 1 h. The resulting solution was stirred 1 h at 0° C. and then 12 h (overnight) at rt. After dilution with a mixture of ether/hexane (1:1, 250 ml) and water (250 mL), the organic phase was separated. Water layer was extracted with a mixture ether/hexane (2×150 mL). Organic extracts were combined and washed with water (4×150 mL), dried over Na₂SO4, and concentrated under vacuum. Crude product was subjected to flash column chromatography (SiO₂-120 g, gradient of Hexane 100% to hexane:AcOEt, 1:1 in 20 minutes) to give 3.18 g (40% yield) of product as an oil. ¹H NMR (400 MHz, Chloroform-d) δ 3.64 (t, J=5.7 Hz, 2H), 3.57 (t, J=6.5 Hz, 2H), 3.51-3.43 (m, 4H), 2.15 (s, 1H), 1.91-1.81 (m, 2H), 1.79-1.61 (m, 6H). ¹³C NMR (151 MHz, Chloroform-d) δ 71.09, 70.24, 62.92, 45.06, 30.46, 29.60, 27.13, 27.02. LC-MS (ESI); m/z: [M+H]⁺ Calcd. for C₈H₁₈ClO₂, 181.0995. Found 181.0990.

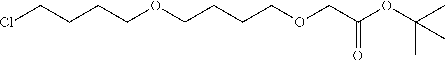 [SMILES: CC(C)(C)OC(=O)COCCCCOCCCCCl]

### tert-Butyl 2-(4-(4-chlorobutoxy)butoxy)acetate (16)

To a solution of 4-(4-chlorobutoxy)butan-1-ol (557 mg, 3.08 mmol) and tert-butyl 2-bromoacetate (0.91 ml, 6.17 mmol) in benzene (12 mL) was added aqueous 50% NaOH (2.47 ml, 30.83 mmol) and TBAHS (1046.76 mg, 3.08 mmol). The reaction mixture was stirred vigorously at room temperature for 4 h. Then the reaction was diluted with EtOAc (50 mL) and water (50 mL), organic layer was separated, washed with water (2×50 mL), dried (Na₂SO₄) and evaporated under vacuum. Crude product was purified by flash column chromatography (SiO₂-80 g, gradient; Hex 100% to Hex:AcOEt, 80:20 in 40 minutes) to give 543 mg of product (60% yield) as an oil. ¹H NMR (400 MHz, Chloroform-d) δ 3.94 (s, 2H), 3.55 (dt, J=13.2, 6.4 Hz, 4H), 3.43 (t, J=6.2 Hz, 4H), 1.92-1.78 (m, 2H), 1.78-1.60 (m, 6H), 1.48 (s, 9H). ¹³C NMR (151 MHz, Chloroform-d) δ 169.95, 81.61, 71.58, 70.71, 69.99, 68.90, 45.17, 29.71, 28.27, 27.25, 26.57, 26.46. LC-MS (ESI): m/z; [M+Na]⁺ Calcd. for C₁₄H₂₇ClO₄Na: 317.1495, Found: 317.1446.

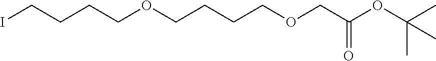 [SMILES: CC(C)(C)OC(=O)COCCCCOCCCCI]

### tert-Butyl 2-(4-(4-iodobutoxy)butoxy)acetate (17)

To a solution of tert-butyl 2-[4-(4-chlorobutoxy)butoxy]acetate (97 mg, 0.33 mmol) in Acetone (15 ml) was added NaI (493.18 mg, 3.29 mmol). The reaction mixture was stirred at reflux temperature for 24 h, then the solvent was removed under vacuum and crude product was dissolved in EtOAc (15 mL) and an aqueous solution of Na₂SO₃ (10%, 10 mL), organic layer was separated, washed with water (10 mL), dried (Na₂SO₄) and evaporated under vacuum. Crude product was pure by NMR (>95% purity, 121 mg, 95% yield) It was used in the next step without any further purification; ¹H NMR (400 MHz, Chloroform-d) δ 3.95 (s, 2H), 3.53 (t, J=6.2 Hz, 2H), 3.42 (t, J=6.2 Hz, 4H), 3.22 (t, J=7.0 Hz, 2H), 1.98-1.84 (m, 2H), 1.74-1.59 (m, 6H), 1.48 (s, 9H). ¹³C NMR (151 MHz, Chloroform-d) δ 169.96, 81.62, 71.59, 70.74, 69.66, 68.92, 30.78, 30.60, 28.28, 26.57, 26.47, 7.11. LC-MS (ESI): m/z; [M+Na]⁺ Calcd. for C₁₄H₂₇IO₄Na: 409.0851, Found: 409.0808.

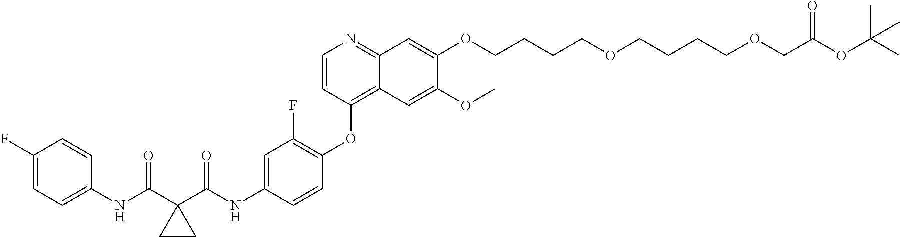 [SMILES: COc1cc2c(Oc3ccc(NC(=O)C4(C(=O)Nc5ccc(F)cc5)CC4)cc3F)ccnc2cc1OCCCCOCCCCOCC(=O)OC(C)(C)C]

### tert-Butyl 2-(4-(4-((4-(2-fluoro-4-(1-((4-fluorophenyl)carbamoyl)cyclopropane-1-carboxa-mido)-phenoxy)-6-methoxyquinolin-7-yl)oxy)butoxy)butoxy)acetate (18)

To a mixture of compound (5) and tert-butyl 2-[4-(4-iodobutoxy)butoxy]acetate (27.51 mg, 0.07 mmol) in N,N-Dimethylformamide (2 mL) was added Cs₂CO₃ (23.2 mg, 0.07 mmol). After stirring at room temperature for 4 hrs, the reaction mixture was diluted with AcOEt (10 mL) and washed with water (5×5 mL), organic phase was evaporated under vacuum. Crude product was purified by PTLC (DCM:MeOH:NH₄OH, 90:9:1) to give 25 mg of product (91% yield). ¹H NMR (400 MHz, DMSO-d6) δ 10.38 (s, 1H), 10.01 (s, 1H), 8.46 (d, J=5.2 Hz, 1H), 7.90 (d, J=13.3 Hz, 1H), 7.64 (dd, J=8.5, 5.2 Hz, 2H), 7.57-7.46 (m, 2H), 7.45-7.36 (m, 2H), 7.15 (t, J=8.8 Hz, 2H), 6.41 (d, J=5.1 Hz, 1H), 4.17 (t, J=6.3 Hz, 2H), 3.95 (s, 3H), 3.92 (s, 2H), 3.49-3.35 (m, 6H), 1.86 (p, J=6.8 Hz, 2H), 1.69 (p, J=6.5 Hz, 2H), 1.60-1.50 (m, 4H), 1.51-1.43 (m, 4H), 1.40 (s, 9H). ¹³C NMR (151 MHz, DMSO-d6) δ 169.52, 168.31, 167.93, 159.32, 158.31 (d, J=240.0 Hz), 153.29 (d, J=245.1 Hz), 151.96, 149.59, 148.81, 146.42, 138.02 (d, J=9.9 Hz), 135.70 (d, J=12.3 Hz), 135.20 (d, J=2.6 Hz), 123.83, 122.47 (d, J=7.9 Hz), 116.93 (d, J=3.3 Hz), 115.13, 114.98, 114.44, 109.00 (d, J=22.7 Hz), 108.47, 101.93, 98.98, 80.58, 70.37, 69.72, 69.58, 68.17, 67.99, 55.79, 31.91, 27.76, 26.04, 25.99, 25.94, 25.48, 15.36. LC-MS (ESI); m/z: [M+H]⁺ Calcd. for C₄₁H₄₈F₂N₃O₉.

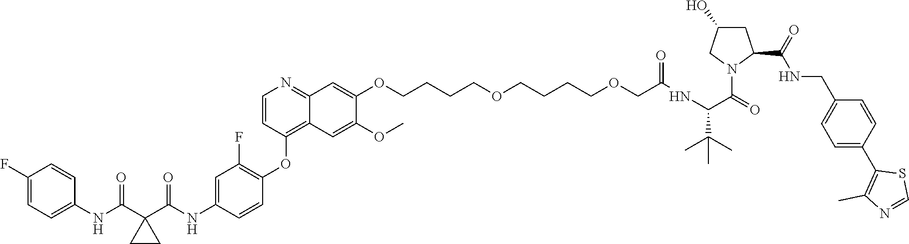 [SMILES: COc1cc2c(Oc3ccc(NC(=O)C4(C(=O)Nc5ccc(F)cc5)CC4)cc3F)ccnc2cc1OCCCCOCCCCOCC(=O)N[C@H](C(=O)N1C[C@H](O)C[C@H]1C(=O)NCc1ccc(-c2scnc2C)cc1)C(C)(C)C]

### N-(3-fluoro-4-((7-(4-(4-(2-(((S)-1-((2S,4R)-4-hydroxy-2-((4-(4-methylthiazol-5-yl)benzyl) carbamoyl)pyrrolidin-1-yl)-3,3-dimethyl-1-oxobutan-2-yl)amino)-2-oxoethoxy)butoxy)buto-xy)-6-methoxyquinolin-4-yl)oxy)phenyl)-N-(4-fluorophenyl)cyclopropane-1,1-dicarboxa-mide (PROTAC 50)

A solution of tert-butyl 2-[4-[4-[[4-[2-fluoro-4-[[1-[(4-fluoro-phenyl)carbamoyl]cyclo-propanecarbonyl]amino]phenoxy]-6-methoxy-7-quinolyl]oxy]butoxy]-butoxy]acetate (10 mg, 0.01 mmol) in a mixture of TFA (0.7 ml, 9.42 mmol) and DCM (2 ml) was stirred for 1 h. Then the solvent was removed under vacuum and crude product was dried under high vacuum for 2 h. Crude product was used in the next step without any further purification (9.2 mg, quantitative yield). LC-MS (ESI); m/z: [M+H]⁺ Calcd. for C₃₇H₄₀F₂N₃O₉, 708.2732. Found 708.2838.

To a solution of 2-[4-[4-[[4-[2-fluoro-4-[[1-[(4-fluorophenyl)carbamoyl]cyclopropanecarbonyl]-amino]phenoxy]-6-methoxy-7-quinolyl]oxy]butoxy]butoxy]acetic acid from above (9.2 mg, 0.01 mmol) and compound (12) (7.29 mg, 0.02 mmol) in N,N-Dimethylformamide (2 ml) was added N,N-Diisopropylethylamine (0.17 ml, 0.98 mmol) and O-(7-Azabenzotriazol-1-yl)-N,N,N′,N′-tetramethyluronium hexafluorophosphate (7.41 mg, 0.02 mmol) at room temperature. The reaction mixture was stirred for 12 h (overnight) at the same temperature. TLC (DCM:MB, 1:1) shows no starting materials. Reaction mixture was diluted with ACOEt (10 mL), washed with water (4×10 mL), dried (Na₂SO₄) and evaporated under vacuum. Crude product was purified by PTLC (DCM:MeOH:NH₄OH, 90:9:1) to give 12 mg of product (82% yield). ¹H NMR (400 MHz, DMSO-d6) δ 10.38 (s, 1H), 10.00 (s, 1H), 8.95 (s, 1H), 8.61 (t, J=6.0 Hz, 1H), 8.44 (d, J=5.2 Hz, 1H), 7.88 (dd, J=13.2, 2.1 Hz, 1H), 7.63 (dd, J=9.1, 5.1 Hz, 2H), 7.55-7.45 (m, 2H), 7.44-7.28 (m, 6H), 7.13 (t, J=8.9 Hz, 2H), 6.39 (d, J=4.8 Hz, 1H), 5.15 (d, J=3.5 Hz, 1H), 4.54 (d, J=9.6 Hz, 1H), 4.48-4.30 (m, 3H), 4.23 (dd, J=15.8, 5.5 Hz, 2H), 4.12 (t, J=6.4 Hz, 2H), 3.92 (s, 3H), 3.90 (s, 2H), 3.68-3.55 (m, 2H), 3.51-3.34 (m, 5H), 2.41 (s, 3H), 2.12-2.00 (m, 1H), 1.93-1.76 (m, 3H), 1.72-1.60 (m, 2H), 1.61-1.52 (m, 4H), 1.50-1.41 (m, 4H), 0.91 (s, 9H). ¹³C NMR (126 MHz, DMSO-d6) δ 172.20, 169.57, 168.92, 168.73, 168.35, 159.75, 158.72 (d, J=240.3 Hz), 153.69 (d, J=245.4 Hz), 152.39, 151.85, 150.01, 149.20, 148.16, 146.79, 139.86, 138.43 (d, J=10.3 Hz), 136.11 (d, J=12.5 Hz), 135.61 (d, J=2.6 Hz), 131.57, 130.12, 129.10, 127.90, 124.23, 122.88 (d, J=7.7 Hz), 117.36, 115.47 (d, J=22.2 Hz), 114.86, 109.42 (d, J=22.8 Hz), 108.86, 102.35, 99.40, 70.14, 70.01, 69.83, 69.33, 68.58, 59.20, 57.05, 56.21, 56.06, 42.12, 38.33, 36.30, 32.34, 26.59, 26.46, 26.42, 26.35, 25.87, 16.34, 15.78. LC-MS (ESI); m/z [M+H]⁺: Calcd. for C₅₉H₆₈F₂N₇O₁₁S, 1120.4665. Found 1120.5044.

### Example 1

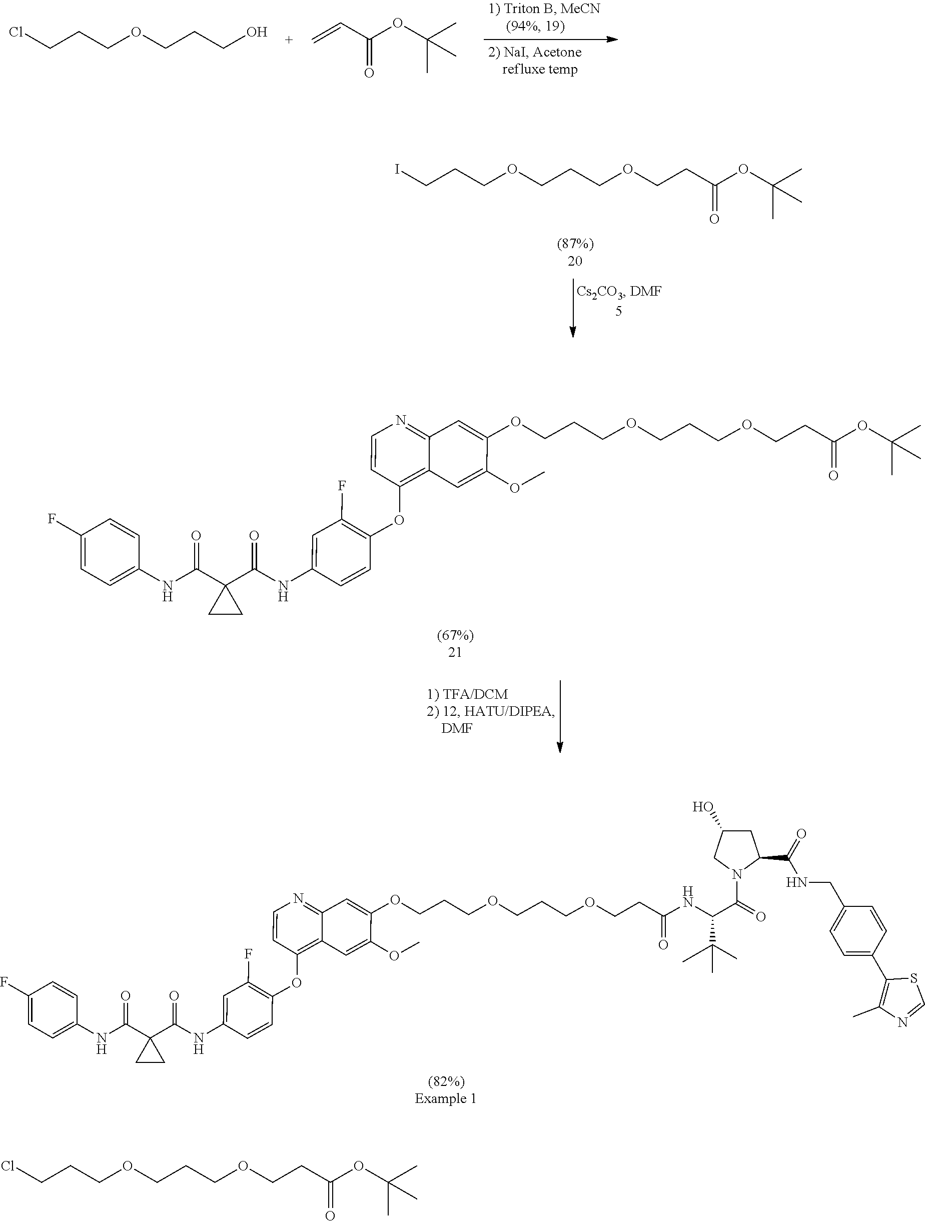

### tert-Butyl 3-(3-(3-chloropropoxy)propoxy)propanoate (19)

3-(3-chloropropoxy)propan-1-ol (66 mg, 0.43 mmol) in acetonitrile (3 mL) was added tert-butyl prop-2-enoate (0.31 ml, 2.16 mmol) followed by Triton B (54 mg, 0.1 mmol, 40% by weight in water). The mixture was stirred at room temperature for 72 hour. The mixture was concentrated under vacuum and crude product was purified by column chromatography (SiO₂, gradient Hex:EtOAc, 95:5 to 9:1) to give 115 mg of product (19) as an oil (94% yield). ¹H NMR (500 MHz, Chloroform-d) δ 3.70-3.59 (m, 4H), 3.59-3.42 (m, 6H), 2.47 (t, J=6.5 Hz, 2H), 2.04-1.96 (m, 2H), 1.82 (p, J=6.3 Hz, 2H), 1.45 (s, 9H). ¹³C NMR (151 MHz, Chloroform-d) δ 171.13, 80.63, 68.02, 67.97, 67.27, 66.64, 42.17, 36.50, 32.88, 30.09, 28.25. LC-MS (ESI); m/z [M+Na]⁺: Calcd. for C₁₃H₂₅ClO₄Na, 303.1339. Found 303.1381 for ³⁵(Cl), and m/z [M+2+Na]⁺305.1385, for ³⁷(Cl).

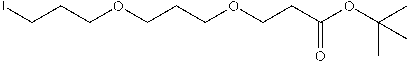 [SMILES: CC(C)(C)OC(=O)CCOCCCOCCCI]

### tert-Butyl 3-(3-(3-iodopropoxy)propoxy)propanoate (20)

To a solution of tert-butyl 3-[3-(3-chloropropoxy)propoxy]propanoate (19) (161 mg, 0.57 mmol) in Acetone (5 ml) was added NaI (429 mg, 2.87 mmol). The reaction mixture was stirred at reflux temperature for 24 hours, then the solvent was removed under vacuum and crude product was dissolved in EtOAc (15 mL), washed with water (10 mL), and with an aqueous solution of Na₂SO₃ (10%, 10 mL). Organic layer was separated, washed with water (10 mL), dried (Na₂SO₄) and evaporated under vacuum. Crude product was pure by NMR (>98% purity, 186 mg, 87% yield), product (20) was used in the next step without any further purification. ¹H NMR (400 MHz, Chloroform-d) δ 3.66 (t, J=6.5 Hz, 2H), 3.57-3.40 (m, 6H), 3.27 (t, J=6.8 Hz, 2H), 2.48 (t, J=6.5 Hz, 2H), 2.08-1.99 (m, 2H), 1.82 (p, J=6.4 Hz, 2H), 1.45 (s, 9H). ¹³C NMR (151 MHz, Chloroform-d) δ 171.13, 80.64, 70.18, 68.01, 67.98, 66.65, 36.50, 33.57, 30.10, 28.26, 3.72. LC-MS (ESI): m/z [M+Na]⁺ Calcd. for C₁₃H₂₅IO₄Na: 395.0695, Found: 395.0719.

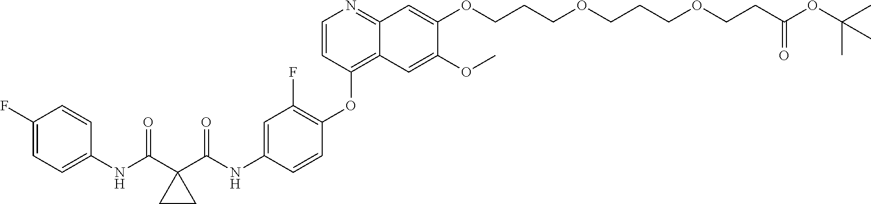 [SMILES: COc1cc2c(Oc3ccc(NC(=O)C4(C(=O)Nc5ccc(F)cc5)CC4)cc3F)ccnc2cc1OCCCOCCCOCCC(=O)OC(C)(C)C]

### tert-Butyl 3-(3-(3-((4-(2-fluoro-4-(1-((4-fluorophenyl)carbamoyl)cyclopropane-1-carboxa-mido)-phenoxy)-6-methoxyquinolin-7-yl)oxy)propoxy)propoxy)propanoate (21)

To a mixture of N1′-[3-fluoro-4-[7-hydroxy-6-methoxy-4-quinolyl)oxy]phenyl]-N1-(4-fluoro-phenyl)cyclopropane-1,1-dicarboxamide (5)² (15 mg, 0.03 mmol) and tert-butyl 3-[3-(3-iodopropoxy)propoxy]propanoate (20) (16.57 mg, 0.04 mmol) in N,N-Dimethylformamide (1 mL) was added Cs₂CO₃ (29.01 mg, 0.09 mmol). After stirring at room temperature for 12 hrs (overnight), the reaction mixture was diluted with AcOEt (20 mL) and washed with water (5×10 mL), organic phase was evaporated under vacuum. Crude product was purified by PTLC (DCM:MeOH:NH₄OH, 92:7:1) to give 15 mg of product (21) (67% yield). ¹H NMR (400 MHz, DMSO-d6) δ 10.39 (s, 1H), 10.01 (s, 1H), 8.46 (d, J=5.2 Hz, 1H), 7.90 (d, J=13.2 Hz, 1H), 7.71-7.58 (m, 2H), 7.51 (d, J=7.4 Hz, 2H), 7.46-7.35 (m, 2H), 7.15 (t, J=8.9 Hz, 2H), 6.41 (d, J=5.1 Hz, 1H), 4.21 (t, J=6.2 Hz, 2H), 3.95 (s, 3H), 3.60-3.37 (m, 8H), 2.37 (d, J=12.2 Hz, 2H), 2.04 (p, J=6.4 Hz, 2H), 1.71 (p, J=6.4 Hz, 2H), 1.47 (s, 4H), 1.37 (s, 9H). ¹³C NMR (151 MHz, dmso) δ 170.45, 168.27, 167.87, 159.29, 158.28 (d, J=240.2 Hz), 153.26 (d, J=245.1 Hz), 151.89, 149.56, 148.82, 146.37, 138.02 (d, J=9.9 Hz), 135.66 (d, J=12.4 Hz), 135.20 (d, J=2.7 Hz), 123.82, 122.43 (d, J=7.9 Hz), 116.90, 115.04 (d, J=22.2 Hz), 114.47, 108.96 (d, J=23.1 Hz), 108.50, 101.95, 99.01, 79.64, 67.07, 66.55, 65.92, 65.45, 55.79, 35.87, 31.93, 29.53, 28.90, 27.76, 27.73, 15.31. LC-MS (ESI): m/z [M+H]⁺ Calcd. for C₄₀H₄₆F₂N₃O₉, 750.3202. Found 750.3509.

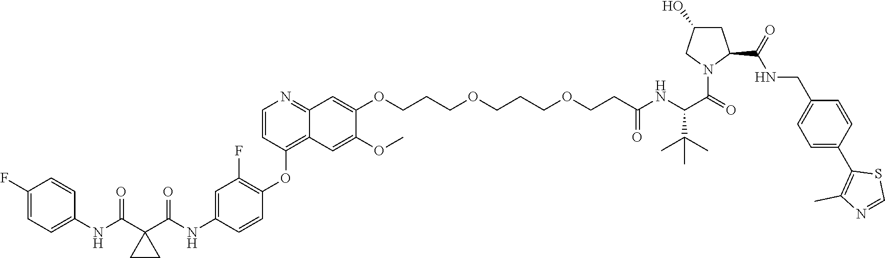 [SMILES: COc1cc2c(Oc3ccc(NC(=O)C4(C(=O)Nc5ccc(F)cc5)CC4)cc3F)ccnc2cc1OCCCOCCCOCCC(=O)N[C@H](C(=O)N1C[C@H](O)C[C@H]1C(=O)NCc1ccc(-c2scnc2C)cc1)C(C)(C)C]

### N-(3-Fluoro-4-((7-(3-(3-(3-(((S)-1-((2S,4R)-4-hydroxy-2-((4-(4-methylthiazol-5-yl)benzyl)carbamoyl)-pyrrolidin-1-yl)-3,3-dimethyl-1-oxobutan-2-yl)amino)-3-oxopropoxy)propoxy)propoxy)-6-methoxyquinolin-4-yl)oxy)phenyl)-N-(4-fluorophenyl)cyclopropane-1,1-dicarboxamide (PROTAC 1)

A solution of 3-[3-[3-[[4-[2-fluoro-4-[[1-[(4-fluorophenyl)carbamoyl]cyclopropanecarbonyl]amino]phenoxy]-6-methoxy-7-quinolyl]oxy]propoxy]propoxy]propanoic acid (21) (15 mg, 0.02 mmol) in a mixture of TFA (1 ml, 13.46 mmol) and Dichloromethane (3 ml) was stirred for 2 h. Then the solvent was removed under vacuum and crude product was dried under high vacuum for 2 h. Crude product was used in the next step without any further purification (13.8 mg, quantitative yield). LC-MS (ESI): m/z [M+H]+ Calcd. for C₃₆H₃₈F₂N₃O₉, 694.2576. Found 694.2324.

To a solution of crude product from above (13.8 mg, 0.02 mmol) and (2S,4R)-1-[(2S)-2-amino-3,3-dimethyl-butanoyl]-4-hydroxy-N-[[4-(4-methylthiazol-5-yl)phenyl]methyl]-pyrrolidine-2-carboxamide; hydrochloride (12)³ (11.15 mg, 0.02 mmol) in N,N-Dimethylformamide (2 ml) was added DIPEA (0.17 ml, 0.99 mmol) and HATU (11.35 mg, 0.03 mmol) at room temperature. The reaction mixture was stirred for 12 h (overnight) at the same temperature. Reaction mixture was diluted with ACOEt (20 mL), washed with water (4×15 mL), dried (Na₂SO₄) and evaporated under vacuum. Crude product was purified by PTLC (DCM:MeOH:NH₄OH, 90:9:1), to give 18 mg of product (82% yield). ¹H NMR (500 MHz, DMSO-d6) δ 10.38 (s, 1H), 10.00 (s, 1H), 8.97 (s, 1H), 8.56 (t, J=6.1 Hz, 1H), 8.46 (d, J=5.2 Hz, 1H), 7.96-7.85 (m, 2H), 7.69-7.59 (m, 2H), 7.51 (d, J=8.8 Hz, 2H), 7.45-7.33 (m, 5H), 7.15 (t, J=8.9 Hz, 2H), 6.41 (d, J=5.1 Hz, 1H), 5.12 (d, J=3.3 Hz, 1H), 4.55 (d, J=9.4 Hz, 1H), 4.43 (ddd, J=10.9, 6.7, 3.3 Hz, 2H), 4.27-4.16 (m, 3H), 3.94 (s, 3H), 3.76-3.33 (m, 10H), 2.58-2.51 (m, 1H), 2.43 (s, 3H), 2.35-2.25 (m, 1H), 2.03 (p, J=5.7 Hz, 3H), 1.95-1.83 (m, 1H), 1.72 (p, J=6.4 Hz, 2H), 1.48 (d, J=3.9 Hz, 4H), 0.92 (s, 9H). ¹³C NMR (126 MHz, dmso) δ 171.89, 169.97, 169.51, 168.26, 167.88, 159.31, 158.27 (d, J=240.1 Hz), 153.23 (d, J=245.1 Hz), 151.90, 151.39, 149.56, 148.75, 147.69, 146.29, 139.47, 137.97 (d, J=9.8 Hz), 135.65 (d, J=12.4 Hz), 135.16 (d, J=2.5 Hz), 131.13, 129.61, 128.61, 127.40, 123.77, 122.43 (d, J=7.9 Hz), 116.90, 115.00 (d, J=22.2 Hz), 114.47, 108.96 (d, J=23.0 Hz), 108.45, 101.94, 99.03, 68.85, 67.16, 67.09, 66.62, 66.54, 65.47, 58.69, 56.35, 56.24, 55.77, 41.64, 37.92, 35.69, 35.36, 31.87, 29.60, 28.89, 26.28, 15.91, 15.31. LC-MS (ESI): m/z [M+H]⁺ Calcd. for C₅₈H₆₆F₂N₇O₁₁S, 1106.4509. Found 1106.4510.

### Example 5

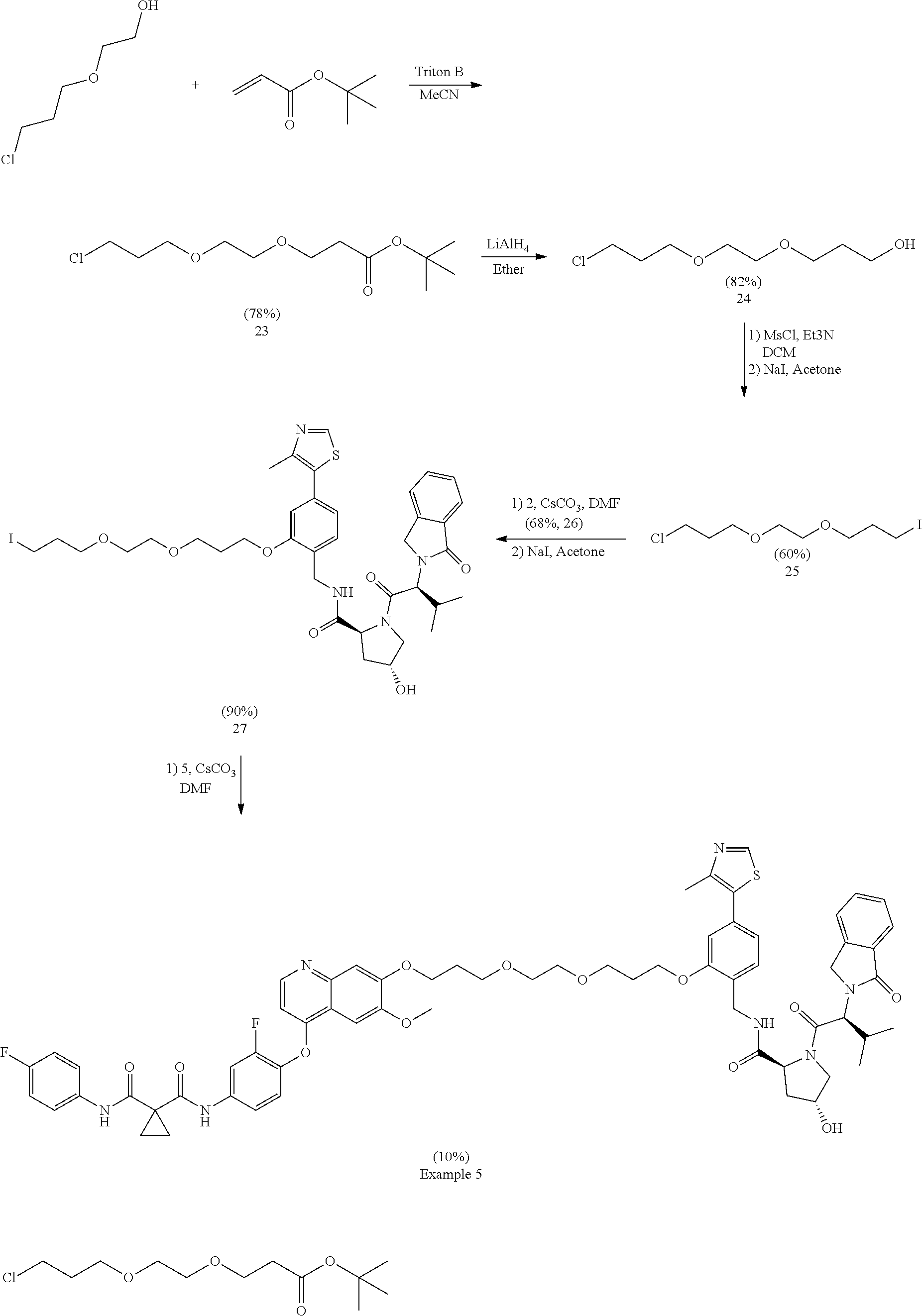

### tert-Butyl 3-(2-(3-chloropropoxy)ethoxy)propanoate (23)

To a solution of 2-(3-chloropropoxy)-ethan-1-ol (1.48 g, 10.68 mmol) in acetonitrile (20 mL) was added tert-butyl prop-2-enoate (7.75 ml, 128.17 mmol) followed by Triton B (447 mg, 1.06 mmol, in 40% by weight in water). The mixture was stirred at room temperature for 12 hours (overnight). The mixture was concentrated in vacuum and crude product was purified by flash chromatography (SiO₂-80 g, gradient Hex:AcOEt, 98:2 to 8:2) to give 2.21 g of product as an oil (77% yield). ¹H NMR (500 MHz, Chloroform-d) δ 3.71 (t, J=6.6 Hz, 2H), 3.63 (t, J=6.4 Hz, 2H), 3.62-3.54 (m, 8H), 2.50 (t, J=6.6 Hz, 2H), 2.02 (p, J=6.2 Hz, 2H), 1.44 (s, 9H). ¹³C NMR (126 MHz, cdcl3) δ 171.02, 80.66, 77.36, 70.46, 67.79, 67.08, 42.09, 36.44, 32.86, 28.25. LC-MS (ESI); m/z: [M+Na]⁺ Calcd. for C₁₂H₂₃ClO₄Na, 289.1182. Found 289.1364.

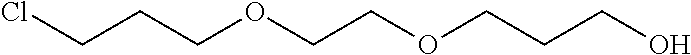 [SMILES: OCCCOCCOCCCCl]

### 3-(2-(3-Chloropropoxy)ethoxy)propan-1-ol (24)

To a solution of the ester (23) (1.21 g, 4.54 mmol) in diethyl ether (30 ml) was added 1M LiAlH₄ (6.8 ml) at 0° C., the reaction mixture was stirred at the same temperature for 30 min., then ice-bath was removed and stirred for 30 min at room temperature. Then Na₂SO₄.10H₂O was added and the mixture was stirred for an additional 30 min. Reaction mixture was filtered under vacuum over a celite pad, and filtrate was evaporated at 50° C. (oil bath). Crude product was purified by flash chromatography (SiO₂-40 g, gradient Hex:EtOAc, 2% to 1:1 in 20 min) to give 0.732 g of product as an oil (82% yield). ¹H NMR (400 MHz, Chloroform-d) δ 3.77 (t, J=5.5 Hz, 2H), 3.72-3.50 (m, 10H), 2.02 (p, J=6.2 Hz, 2H), 1.83 (p, J=5.7 Hz, 2H). ¹³C NMR (151 MHz, cdcl3) δ 70.56, 70.29, 70.23, 67.67, 61.96, 42.02, 32.69, 31.94. LC-MS m/z: [M+H]⁺ Calcd. for C₈H₁₈ClO₃, 197.0944. Found 197.1085.

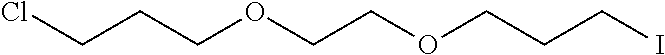 [SMILES: ClCCCOCCOCCCI]

### 1-Chloro-3-(2-(3-iodopropoxy)ethoxy)propane (25)

To a solution of 3-(2-(3-chloro-propoxy)ethoxy)propan-1-ol (24) (200 mg, 1.02 mmol) in Dichloromethane (5 ml) was added TEA (0.42 ml, 3.05 mmol), then reaction mixture was cooled to 0° C. (water ice/acetone bath) and mesyl chloride (0.09 ml, 1.22 mmol) was added dropwise. The reaction mixture was stirred for 1 h at the same temperature. By TLC no starting material (Hex:AcOEt, 3:7). Reaction mixture was poured into an aqueous solution of NaHCO₃ (20 mL) and product extracted with DCM (20 mL, 2×), the organic extracts were combined, dried (Na₂SO₄), and evaporated under vacuum. The crude product (mesylate) was used in the next step without any further purification (>95% pure by NMR)): ¹H NMR (500 MHz, Chloroform-d) δ 4.35 (t, J=6.2 Hz, 2H), 3.64 (t, J=6.4 Hz, 2H), 3.62-3.53 (m, 8H), 3.01 (s, 3H), 2.02 (pd, J=6.1, 3.8 Hz, 4H).

Crude mixture from previous step was diluted in acetonitrile (5 ml) and NaI (228.65 mg, 1.53 mmol) was added, the reaction mixture was stirred at 70° C. for 3 h. By TLC (Hex:AcOEt, 7:3) and NMR small amount of masylate, the reaction was poured into an aqueous solution of Na₂S₂O₃ (10%, 20 mL) and product was extracted with DCM (2×20 mL). Organic extracts were combined, dried (NA₂SO₄) and evaporated under vacuum. Crude product was purified by flash chromatography (SiO₂-25 g, grad. Hex:AcOEt, 2 to 20% in 15 min), to give 187 mg of product as an oil (60% yield), small amount of bis-iodo compound by LC-MS (˜10%). ¹H NMR (400 MHz, Chloroform-d) δ 3.65 (t, J=6.4 Hz, 2H), 3.63-3.56 (m, 6H), 3.53 (t, J=5.9 Hz, 2H), 3.28 (t, J=6.7 Hz, 2H), 2.11-1.98 (m, 4H). ¹³C NMR (151 MHz, cdcl3) δ 70.67, 70.46, 70.44, 67.76, 42.12, 33.47, 32.81, 3.63. LC-MS (ESI); m/z: [M+H]⁺ Calcd. for C₈H₁₇ClIO₂, 306.9961. Found 307.1626.

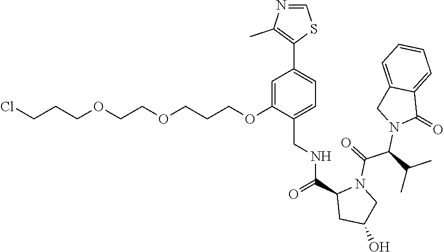 [SMILES: Cc1ncsc1-c1ccc(CNC(=O)[C@@H]2C[C@@H](O)CN2C(=O)[C@H](C(C)C)N2Cc3ccccc3C2=O)c(OCCCOCCOCCCCl)c1]

### (2S,4R)—N-(2-(3-(2-(3-chloropropoxy)ethoxy)propoxy)-4-(4-methylthiazol-5-yl)benzyl)-4-hydroxy-1-((S)-3-methyl-2-(1-oxoisoindolin-2-yl)butanoyl)pyrrolidine-2-carboxamide (26)

To a mixture of (2S,4R)-4-hydroxy-N-[[2-hydroxy-4-(4-methylthiazol-5-yl)phenyl]methyl]-1-[(2S)-3-methyl-2-(1-oxoisoindolin-2-yl)butanoyl]pyrrolidine-2-carboxamide (2)¹ (75 mg, 0.13 mmol) and 1-chloro-3-(2-(3-iodopropoxy)-ethoxy)propane (25) (54.48 mg, 0.18 mmol) in DMF (1 mL) was added Cs₂CO₃ (89.08 mg, 0.27 mmol). After stirring at room temperature for 2 hrs, the reaction mixture was diluted with AcOEt (10 mL) and washed with water (5×10 mL), organic phase was dried (Na₂SO₄), and evaporated under vacuum. Crude product was purified by PTLC (DCM:MEOH:NH₄OH, 90:9:1) to give 68 mg of product (68% yield): ¹H NMR (400 MHz, Chloroform-d) δ 8.67 (s, 1H), 7.73 (d, J=7.7 Hz, 1H), 7.49 (td, J=7.4, 1.2 Hz, 1H), 7.39 (dt, J=7.5, 3.6 Hz, 2H), 7.33-7.23 (m, 2H), 6.95 (dd, J=7.6, 1.6 Hz, 1H), 6.89 (s, 1H), 4.84-4.66 (m, 2H), 4.62 (t, J=7.8 Hz, 1H), 4.59-4.28 (m, 6H), 4.12 (t, J=6.1 Hz, 2H), 3.81-3.63 (m, 4H), 3.63-3.49 (m, 6H), 3.41 (bs, 1H), 2.52 (s, 3H), 2.50-2.29 (m, 2H), 2.15 (p, J=6.1 Hz, 2H), 2.02-1.89 (m, 2H), 0.91 (d, J=6.5 Hz, 3H), 0.86 (d, J=6.6 Hz, 3H). ¹³C NMR (101 MHz, cdcl3) δ 170.66, 170.37, 169.59, 156.87, 150.41, 148.59, 142.17, 132.36, 131.92, 131.85, 131.65, 129.39, 128.08, 126.42, 123.88, 122.94, 121.67, 112.19, 70.43, 70.40, 70.02, 67.93, 67.73, 65.26, 58.74, 58.67, 56.10, 47.57, 42.01, 38.99, 36.04, 32.71, 29.69, 29.00, 19.15, 16.24. LC-MS (ESI); m/z: [M+H]⁺ Calcd. for C₃₇H₄₈C1N₄O₇S, 727.2932. Found 727.4574.

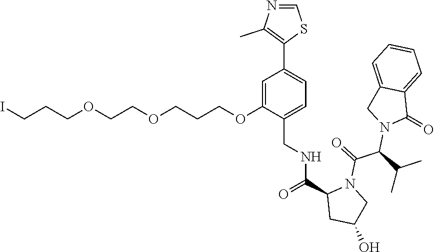 [SMILES: Cc1ncsc1-c1ccc(CNC(=O)[C@@H]2C[C@@H](O)CN2C(=O)[C@H](C(C)C)N2Cc3ccccc3C2=O)c(OCCCOCCOCCCI)c1]

### (2S,4R)-4-Hydroxy-N-(2-(3-(2-(3-iodopropoxy)ethoxy)propoxy)-4-(4-methylthiazol-5-yl)-benzyl)-1-((S)-3-methyl-2-(1-oxoisoindolin-2-yl)butanoyl)pyrrolidine-2-carboxamide (27)

To a solution of (2S,4R)—N-[[2-[3-[2-(3-chloropropoxy)ethoxy]propoxy]-4-(4-methylthiazol-5-yl)phenyl]methyl]-4-hydroxy-1-[(2S)-3-methyl-2-(1-oxoisoindolin-2-yl)butanoyl]pyrrolidine-2-carboxamide (26) (69 mg, 0.09 mmol) in Acetone (10 ml) was added NaI (71.1 mg, 0.47 mmol). The reaction mixture was stirred at reflux temperature for 24 h, then the solvent was removed under vacuum and crude product was dissolved in EtOAc (15 mL) and an aqueous solution of Na₂SO₃ (10%, 10 mL), organic layer was separated, washed with water (10 mL), dried (Na₂SO₄) and evaporated under vacuum. Crude product was pure by NMR (>95% purity, 70 mg, 90% yield), it was used in the next step without any further purification; ¹H NMR (500 MHz, Chloroform-d) δ 8.67 (s, 1H), 7.75 (dt, J=7.7, 4.4 Hz, 1H), 7.54-7.47 (m, 1H), 7.44-7.37 (m, 2H), 7.29 (d, J=7.7 Hz, 2H), 6.96 (d, J=7.7 Hz, 1H), 6.90 (s, 1H), 4.80-4.69 (m, 2H), 4.64 (t, J=7.8 Hz, 1H), 4.56-4.34 (m, 5H), 4.13 (t, J=5.7 Hz, 2H), 3.74-3.62 (m, 3H), 3.64-3.54 (m, 4H), 3.50 (t, J=5.9 Hz, 2H), 3.22 (t, J=6.7 Hz, 2H), 2.52 (s, 3H), 2.52-2.44 (m, 1H), 2.40 (dtd, J=13.2, 6.7, 3.4 Hz, 1H), 2.22-2.10 (m, 2H), 2.09-1.96 (m, 3H), 0.90 (d, J=6.5 Hz, 3H), 0.87 (d, J=6.6 Hz, 3H). ¹³C NMR (126 MHz, cdcl3) δ 170.45, 170.27, 169.49, 156.74, 150.26, 148.46, 142.05, 132.23, 131.78, 131.73, 131.54, 129.27, 127.96, 126.26, 123.77, 122.82, 121.53, 112.06, 70.51, 70.28, 69.92, 67.80, 65.12, 58.61, 58.44, 55.93, 47.43, 41.87, 38.88, 35.77, 33.23, 29.56, 28.78, 19.03, 16.13, 3.37. LC-MS (ESI): m/z; [M+H]⁺ Calcd. for C₃₇H₄₈IN₄O₇S: 819.2288, Found: 819.2384.

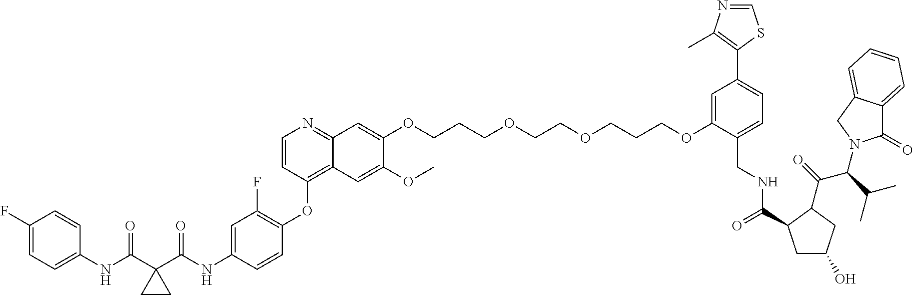 [SMILES: COc1cc2c(Oc3ccc(NC(=O)C4(C(=O)Nc5ccc(F)cc5)CC4)cc3F)ccnc2cc1OCCCOCCOCCCOc1cc(-c2scnc2C)ccc1CNC(=O)[C@@H]1C[C@@H](O)CC1C(=O)[C@H](C(C)C)N1Cc2ccccc2C1=O]

### N-(3-fluoro-4-((7-(3-(2-(3-(2-(((2S,4R)-4-hydroxy-1-((S)-3-methyl-2-(1-oxoisoindolin-2-yl)-butanoyl)pyrrolidine-2-carboxamido)methyl)-5-(4-methylthiazol-5-yl)phenoxy)propoxy)-ethoxy)propoxy)-6-methoxyquinolin-4-yl)oxy)phenyl)-N-(4-fluorophenyl)cyclopropane-1,1-dicarboxamide (PROTAC 5)

To a mixture of (2S,4R)-4-hydroxy-N-[[2-[3-[2-(3-iodopropoxy)ethoxy]propoxy]-4-(4-methylthiazol-5-yl)phenyl]methyl]-1-[(2S)-3-methyl-2-(1-oxoisoindolin-2-yl)butanoyl]pyrrolidine-2-carboxamide (27) (15.8 mg, 0.02 mmol) and N1′-[3-fluoro-4-[(7-hydroxy-6-methoxy-4-quinolyl)oxy]phenyl]-N1-(4-fluorophenyl)cyclopropane-1,1-dicarboxamide (5)² (9.75 mg, 0.02 mmol) in DMF (1 mL) was added Cs₂CO₃ (12.57 mg, 0.04 mmol). After stirring at room temperature for 12 hrs (overnight), the reaction mixture was diluted with AcOEt (10 mL) and washed with brine (5×10 mL), organic phase was dried (Na₂SO₄, and evaporated under vacuum. Crude product was purified by PTLC (DCM:MEOH:NH₄OH, 90:9:1) to give 2.5 mg of product (10% yield). ¹H NMR (600 MHz, DMSO-d6) δ 10.39 (s, 1H), 10.01 (s, 1H), 8.96 (s, 1H), 8.45 (d, J=4.8 Hz, 1H), 8.38 (t, J=5.4 Hz, 1H), 7.90 (d, J=13.0 Hz, 1H), 7.70 (d, J=7.5 Hz, 1H), 7.68-7.28 (m, 10H), 7.15 (t, J=8.5 Hz, 2H), 7.04-6.92 (m, 2H), 6.40 (d, J=5.0 Hz, 1H), 5.09 (d, J=3.0 Hz, 1H), 4.70 (d, J=10.8 Hz, 1H), 4.54 (d, J=18.1 Hz, 1H), 4.49-4.37 (m, 2H), 4.35-4.26 (m, 2H), 4.23 (dd, J=16.3, 6.0 Hz, 1H), 4.17 (t, J=6.1 Hz, 2H), 4.09 (t, J=5.7 Hz, 2H), 3.93 (s, 2H), 3.81-3.65 (m, 2H), 3.64-3.46 (m, 8H), 2.45 (s, 3H), 2.35-2.28 (m, 1H), 2.07-1.86 (m, 6H), 1.47 (d, J=5.6 Hz, 4H), 0.95 (d, J=6.2 Hz, 3H), 0.72 (d, J=6.3 Hz, 3H). ¹³C NMR (151 MHz, dmso) δ 171.92, 168.70, 168.50, 168.32, 167.88, 159.73, 159.50, 157.91, 156.27, 154.48, 152.86, 152.31, 151.83, 149.96, 149.20, 148.29, 146.72, 142.61, 138.46, 138.40, 136.11, 136.02, 135.62, 135.60, 131.99, 131.78, 131.68, 131.41, 128.32, 128.19, 127.41, 124.23, 124.02, 123.42, 122.89, 122.83, 121.24, 117.32, 115.53, 115.38, 114.88, 112.07, 109.46, 109.31, 108.86, 102.35, 99.43, 70.02, 70.00, 69.03, 67.32, 67.29, 65.82, 65.18, 59.10, 58.19, 56.20, 55.84, 47.22, 38.52, 37.47, 32.34, 29.51, 29.33, 28.80, 19.28, 19.03, 16.41, 15.74. LC-MS (ESI); m/z: [M+H]⁺ Calcd. for C₆₄H₆₈F₂N₇O₁₂S, 1196.4614. Found 1196.4210.

Linker Synthesis:

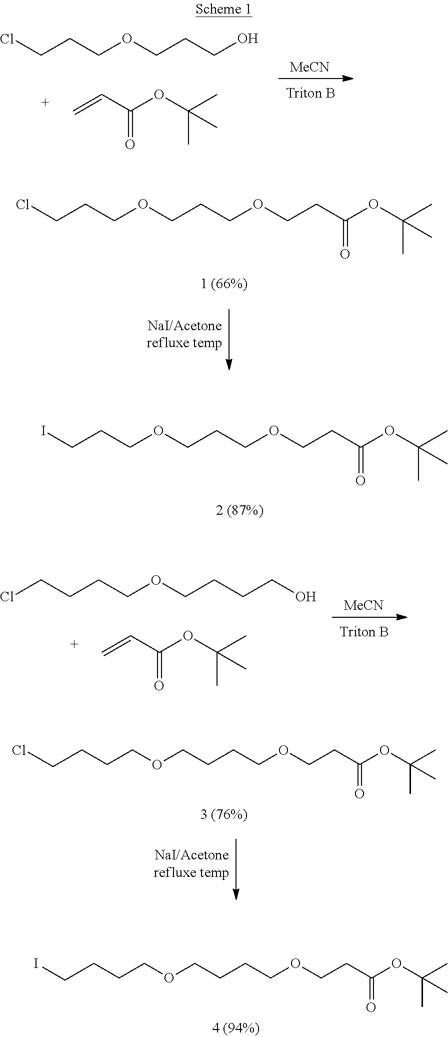

### Synthesis of tert-butyl 3-(3-(3-chloropropoxy)propoxy)propanoate (1)

3-(3-chloropropoxy)propan-1-ol (66 mg, 0.43 mmol) in acetonitrile (3 mL) was added tert-butyl prop-2-enoate (0.31 ml, 2.16 mmol) followed by Triton B (54.2 mg, OA mmol, 40% by weight in water). The mixture was stirred at room temperature for 72 hour (over the weekend). The mixture was concentrated in vacuum and crude product was purified by CC (SiO₂, gradient Hex:AcOEt, 95:5 to 9:1) to give 115 mg of product as an oil (95% yield). ¹H NMR (500 MHz, Chloroform-d) δ 3.70-3.59 (m, 411), 3.59-3.42 (m, 6H), 2.47 (t. J=6.5 Hz, 2H), 2.04-1.96 (m, 2H), 1.82 (p, J=6.3 Hz, 2H), 1.45 (s, 9H). LC-MS (ESI); m/z [M+Na]⁺: Calcd. for C₁₃H₂₅ClO₄Na, 303.1339. Found 303.1381.

### Synthesis of tert-butyl 3-(3-(3-iodopropoxy)propoxy)propanoate (2)

To a solution of tert-butyl 3-[3-(3-chloropropoxy)propoxy]propanoate (161 mg, 0.57 mmol) in Acetone (5 ml) was added NaI (429.74 mg, 2.87 mmol). The reaction mixture was stirred at reflux temperature for 24 h, then the solvent was removed under vacuum and crude product was dissolved in EtOAc (15 mL) and an aqueous solution of Na₂SO₃ (10%, 10 mL), organic layer was separated, washed with water (10 mL), dried (Na₂SO₄) and evaporated under vacuum. Crude product was pure by NMR (>95% purity, 186 mg, 87% yield) no further purification. ¹H NMR (400 MHz, Chloroform-d) δ 3.66 (t, J=6.5 Hz, 2H), 3.57-3.40 (m, 6H), 3.27 (t, J=6.8 Hz, 2H), 2.48 (t, J=6.5 Hz, 2H), 2.08-1.99 (m, 2H), 1.82 (p, J=6.4 Hz, 2H), 1.45 (s, 9H). LC-MS (ESI); m/z [M+Na]⁺: Calcd. for C₁₃H₂₅IO₄Na. 395.0695, Found: 395.0719.

### tert-butyl 3-(4-(4-chlorobutoxy)butoxy)propanoate (3)

4-(4-chlorobutoxy)butan-1-ol (400 mg, 2.21 mmol) in acetonitrile (20 mL) was added tert-butyl prop-2-enoate (1.61 ml, 11.07 mmol) followed by Triton B (40%, 277.72 mg, 0.66 mmol, in 40% by weight in water). The mixture was stirred at room temperature for 12 hours (overnight). The mixture was concentrated in vacuum and crude product was purified by CC (SiO₂, gradient Hex:AcOEt, 98:2 to 95:5) to give 656 mg of product as an oil (77% yield). ¹H NMR (400 MHz, Chloroform-d) δ 3.65 (t, J=6.5 Hz, 2H), 3.56 (t, J=6.6 Hz, 2H), 3.52-3.33 (m, 6H), 2.47 (t, =6.5 Hz, 2H), 1.96-1.78 (m, 1H), 1.80-1.65 (m, 2H), 1.67-1.54 (m, 4H), 1.45 (s, 9H). LC-MS (ESI); [M+H]⁺ Calcd. for C₁₅H₂₉ClO₄Na, 331.1652. Found 331.1768.

### tert-butyl 3-(4-(4-iodobutoxy)butoxy)propanoate (4)

To a solution of tert-butyl 3-[4-(4-chlorobutoxy)butoxy]propanoate (200 mg, 0.65 mmol) in Acetone (5 ml) was added NaI (485.34 mg, 3.24 mmol). The reaction mixture was stirred at reflux temperature for 24 h, then the solvent was removed under vacuum and crude product was dissolved in EtOAc (15 mL) and an aqueous solution of Na₂SO₃ (10%, 10 mL), organic layer was separated, washed with water (10 mL), dried (Na₂SO₄) and evaporated under vacuum. Crude product was pure by NMR (>95% purity, 245 mg, 94% yield) no further purification. ¹H NMR (400 MHz, Chloroform-d) δ 3.65 (t, J=6.5 Hz, 2H), 3.42 (dq, J=11.2, 5.3 Hz, 6H), 3.21 (t, J=7.0 Hz, 2H), 2.48 (s, 2H), 1.91 (p, J=7.1 Hz, 2H), 1.73-1.53 (m, 6H), 1.45 (s, 9H). LC-MS (ESI): m/z; [M+Na]⁺ Calcd. for C₁₅H₂₉IO₄Na: 423.1008, Found: 423.1165.

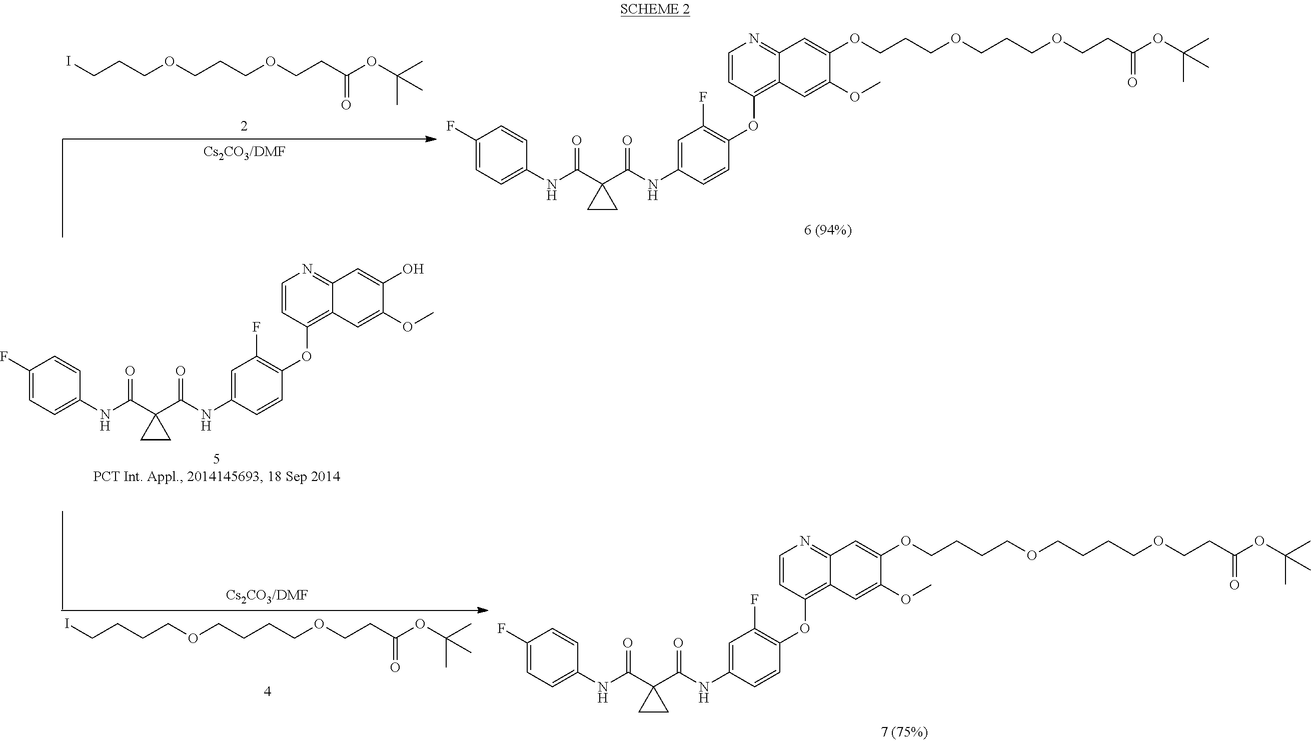

### tert-butyl 3-(3-(3-((4-(2-fluoro-4-(1-((4-fluorophenyl)carbamoyl)cyclopropane-1-carboxamido)phenoxy)-6-methoxyquinolin-7-yl)oxy)propoxy)propoxy)propanoate (6)

To a mixture of N1′-[3-fluoro-4-[(7-hydroxy-6-methoxy-4-quinolyl)oxy]phenyl]-N1-(4-fluorophenyl)cyclopropane-1,1-dicarboxamide (20 mg, 0.04 mmol) and tert-butyl 3-[3-(3-iodopropoxy)propoxy]propanoate (22 mg, 0.59 mmol) in N,N-Dimethylformamide (1 mL) was added Cs₂CO₃ (38.67 mg, 0.119 mmol). After stirring at room temperature for 4 hrs, the reaction mixture was diluted with AcOEt (10 mL) and washed with water (5×10 mL), organic phase was evaporated under vacuum. Crude product was purified by PTLC (DCM:MEOH:NH₄OH, 90:9:1) to give 28 mg of product (94% yield). ¹H NMR (400 MHz, DMSO-d6) δ 10.39 (s, 1H), 10.01 (s, 1H), 8.46 (d, J=5.2 Hz, 1H), 7.90 (d, J=13.2 Hz, 1H), 7.71-7.58 (m, 2H), 7.51 (d, J=7.4 Hz, 2H), 7.46-7.35 (m, 2H), 7.15 (t, J=8.9 Hz, 2H), 6.41 (d, J=5.1 Hz, 1H), 4.21 (t, J=6.2 Hz, 2H), 3.95 (s, 3H), 3.60-3.37 (m, 8H), 2.37 (d, J=12.2 Hz, 2H), 2.04 (p, J=6.4 Hz, 2H), 1.71 (p, J=6.4 Hz, 2H), 1.47 (s, 4H), 1.37 (s, 9H). LC-MS (ESI); m/z: [M+H]⁺ Calcd. for C₄₀H₄₆F₂N₃O₉, 750.3202. Found 750.3509.

### tert-butyl 3-(4-(44(4-(2-fluoro-4-(1-((4-fluorophenyl)carbamoyl)cyclopropane-1-carboxamido)phenoxy)-6-methoxyquinolin-7-yl)oxy)butoxy)butoxy)propanoate (7)

To a mixture of N1′-[3-fluoro-4-[(7-hydroxy-6-methoxy-4-quinolyl)oxy]phenyl]-N1-(4-fluorophenyl)cyclopropane-1,1-dicarboxamide (15 mg, 0.03 mmol) and tert-butyl 3-[3-(3-iodopropoxy)propoxy]propanoate (16.57 mg, 0.04 mmol) in N,N-Dimethylformamide (1 mL) was added Cs2CO3 (29.01 mg, 0.09 mmol). After stirring at room temperature for 12 hrs (overnight), the reaction mixture was diluted with AcOEt (20 mL) and washed with water (5×10 mL), organic phase was evaporated under vacuum. Crude product was purified by PTLC (DCM:MEOH:NH₄OH, 90:9:1) to give 28 mg of product (76% yield). ¹H NMR (400 MHz, Chloroform-d) δ 10.03 (s, 1H), 8.46 (d, J=5.3 Hz, 1H), 8.37 (s, 1H), 7.76 (dd, J=12.0, 2.4 Hz, 1H), 7.55 (s, 1H), 7.49-7.41 (m, 2H), 7.39 (s, 1H), 7.31-7.15 (m, 2H), 7.06 (t, J=8.6 Hz, 2H), 6.38 (dd, J=5.3, 1.1 Hz, 1H), 4.20 (t, J=6.7 Hz, 2H), 4.03 (s, 3H), 3.64 (t, J=6.5 Hz, 2H), 3.49 (t, J=6.4 Hz, 2H), 3.44 (h, J=3.7, 3.2 Hz, 4H), 2.47 (t, J=6.5 Hz, 2H), 2.07-1.94 (m, 2H), 1.87-1.73 (m, 4H), 1.70-1.54 (m, 6H), 1.44 (s, 9H). HRMS (ESI); m/z: [M+H]⁺ Calcd. for C₄₂H₅₀F₂N₃O₉, 778.3515. Found 778.3487.

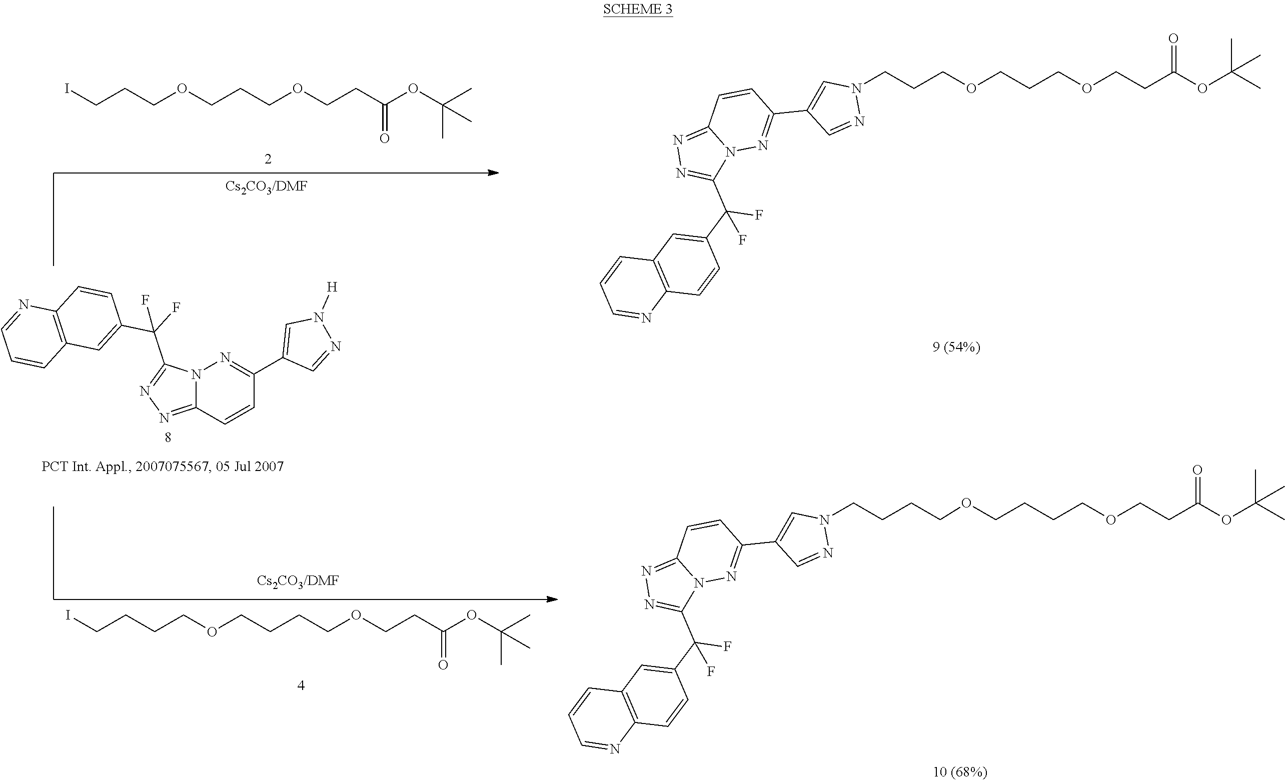 [SMILES: FC(F)(c1ccc2ncccc2c1)c1nnc2ccc(-c3cn[nH]c3)nn12 ; CC(C)(C)OC(=O)CCOCCCOCCCI ; CC(C)(C)OC(=O)CCOCCCOCCCn1cc(-c2ccc3nnc(C(F)(F)c4ccc5ncccc5c4)n3n2)cn1 ; CC(C)(C)OC(=O)CCOCCCCOCCCCI ; CC(C)(C)OC(=O)CCOCCCCOCCCCn1cc(-c2ccc3nnc(C(F)(F)c4ccc5ncccc5c4)n3n2)cn1]

### tert-butyl 3-(3-(3-(4-(3-(difluoro(quinolin-6-yl)methyl)-[1,2,4]triazolo[4,3-b]pyridazin-6-yl)-1H-pyrazol-1-yl)propoxy)propoxy)propanoate (9)

To a suspension of 6-[difluoro-[6-(1H-pyrazol-4-yl)-[1,2,4]triazolo[4,3-b]pyridazin-3-yl]methyl]quinoline (35 mg, 0.1 mmol) in Acetonitrile (3 ml) was added tert-butyl 3-[3-(3-iodopropoxy)propoxy]propanoate (35.86 mg, 0.1 mmol) and Cs2CO3 (62.77 mg, 0.19 mmol) at room temperature, then the reaction mixture was heated to reflux temperature for 12 h (overnight). The reaction mixture was diluted with DCM (20 mL) and water (10 mL), organic extract was separated, dried (Na₂SO₄) and evaporated under vacuum. Crude product was purified by CC (SiO2, gradient DCM 100% to DCM:MEOH:NH₄OH, 90:9:1) to give 32 mg of pure product (54% yield). ¹H NMR (400 MHz, DMSO-d6) δ 9.02 (dd, J=4.3, 1.7 Hz, 1H), 8.62 (dd, J=7.9, 1.8 Hz, 1H), 8.57-8.42 (m, 3H), 8.19 (d, J=8.9 Hz, 1H), 8.06 (s, 1H), 8.02 (dd, J=8.9, 2.1 Hz, 1H), 7.84 (d, J=9.8 Hz, 1H), 7.66 (dd, J=8.3, 4.2 Hz, 1H), 4.23 (t, J=6.9 Hz, 2H), 3.52 (t, J=6.1 Hz, 2H), 3.43-3.22 (m, 6H), 2.37 (t, J=6.1 Hz, 2H), 2.03 (p, J=6.6 Hz, 2H), 1.69 (p, J=6.4 Hz, 2H), 1.35 (s, 9H). LC-MS (ESI); m/z: [M+H]⁺ Calcd. for C₃₁H₃₆F₂N₇O₄, 608.2796. Found 608.2820.

### tert-butyl 3-(4-(4-(4-(3-(difluoro(quinolin-6-yl)methyl)-[1,2,4]triazolo[4,3-b]pyridazin-6-yl)-1H-pyrazol-1-yl)butoxy)butoxy)propanoate (10)

To a suspension of 6-[difluoro-[6-(1H-pyrazol-4-yl)-[1,2,4]triazolo[4,3-b]pyridazin-3-yl]methyl]quinoline (35 mg, 0.1 mmol) in Acetonitrile (3 ml) was added tert-butyl 3-[4-(4-iodobutoxy)butoxy]propanoate (38.56 mg, 0.1 mmol) and Cs₂CO₃ (62.77 mg, 0.19 mmol) at room temperature, then the reaction mixture was heated to reflux temperature for 12 h (overnight). The reaction mixture was diluted with DCM (20 mL) and water (10 mL), organic extract was separated, dried (Na₂SO₄) and evaporated under vacuum. Crude product was purified by CC (SiO₂, gradient DCM 100% to DCM:MEOH:NH₄OH, 90:9:1) to give 42 mg of pure product (68% yield). ¹H NMR (600 MHz, Chloroform-d) δ 9.00 (d, J=3.9 Hz, 1H), 8.34-8.19 (m, 3H), 8.12 (d, J=9.7 Hz, 1H), 8.07 (d, J=8.8 Hz, 1H), 7.97 (d, J=6.6 Hz, 2H), 7.52-7.45 (m, 1H), 7.40 (d, J=9.7 Hz, 1H), 4.23 (t, J=7.1 Hz, 2H), 3.64 (t, J=6.4 Hz, 2H), 3.51-3.36 (m, 6H), 2.46 (t, J=6.4 Hz, 2H), 2.00 (p, J=7.2 Hz, 2H), 1.74-1.51 (m, 6H), 1.43 (s, 9H). LC-MS (ESI); m/z: [M+H]⁺ Calcd. for C₃₃H₄₀F₂N₇O₄, 636.3109. Found 636.3508.

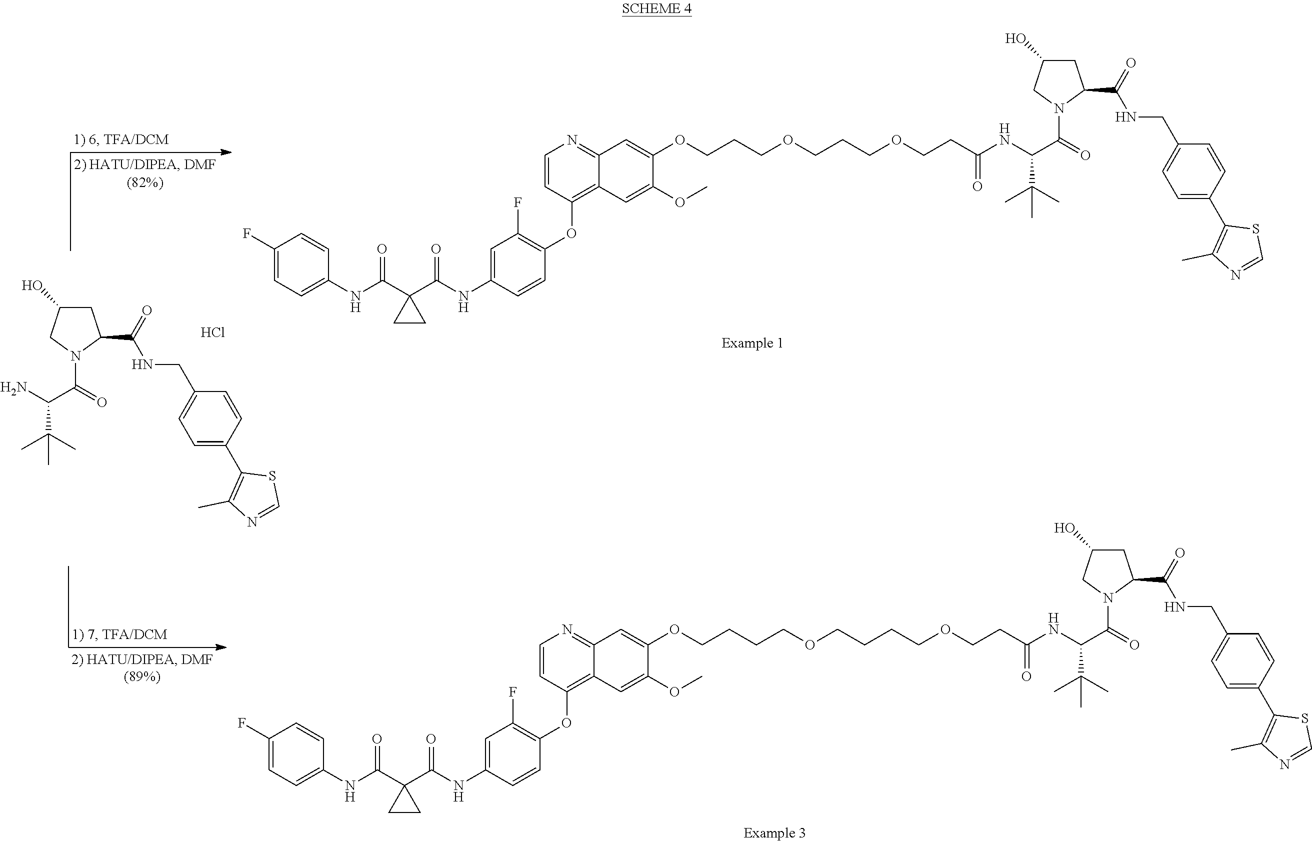 [SMILES: Cc1ncsc1-c1ccc(CNC(=O)[C@@H]2C[C@@H](O)CN2C(=O)[C@@H](N)C(C)(C)C)cc1 ; Cl ; COc1cc2c(Oc3ccc(NC(=O)C4(C(=O)Nc5ccc(F)cc5)CC4)cc3F)ccnc2cc1OCCCOCCCOCCC(=O)N[C@H](C(=O)N1C[C@H](O)C[C@H]1C(=O)NCc1ccc(-c2scnc2C)cc1)C(C)(C)C ; COc1cc2c(Oc3ccc(NC(=O)C4(C(=O)Nc5ccc(F)cc5)CC4)cc3F)ccnc2cc1OCCCCOCCCCOCCC(=O)N[C@H](C(=O)N1C[C@H](O)C[C@H]1C(=O)NCc1ccc(-c2scnc2C)cc1)C(C)(C)C]

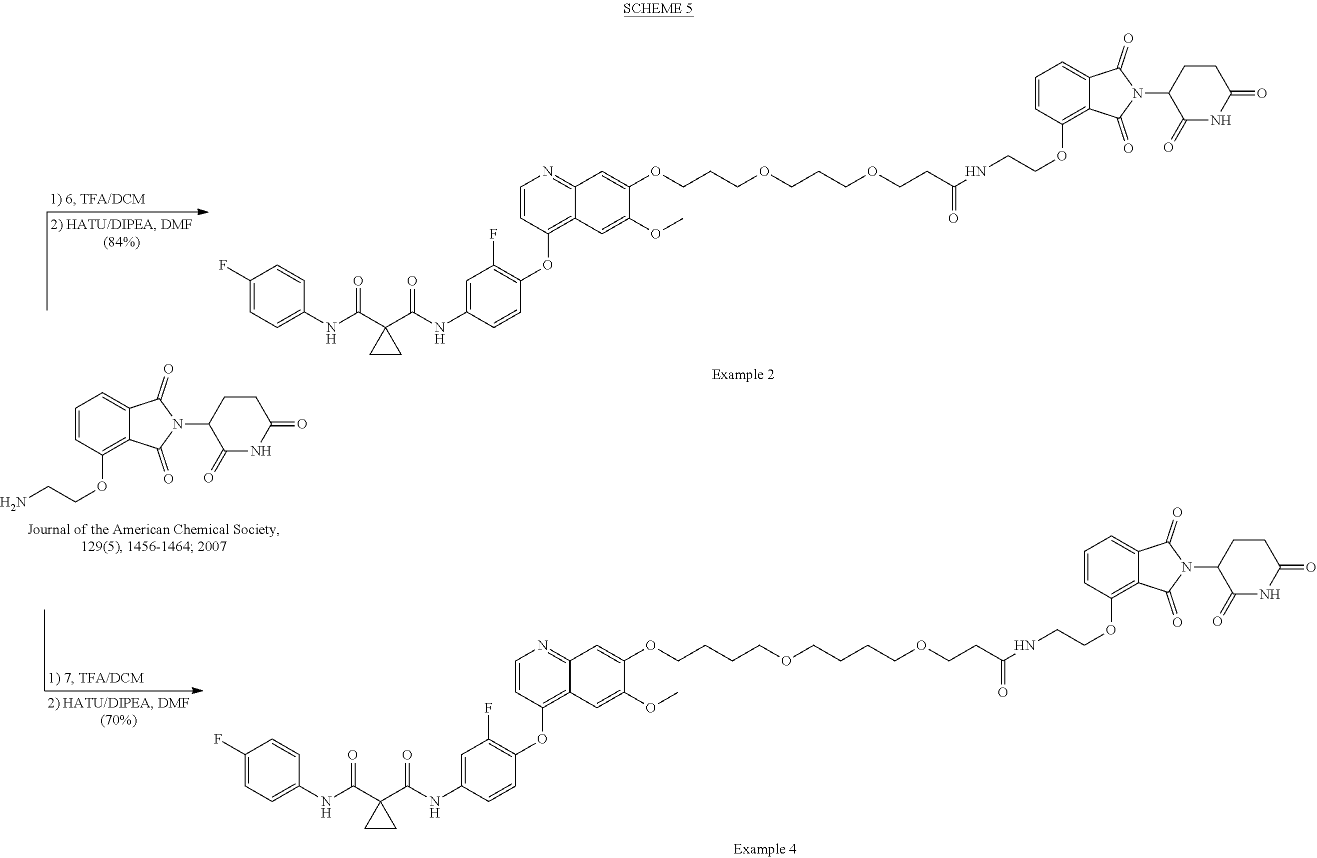 [SMILES: COc1cc2c(Oc3ccc(NC(=O)C4(C(=O)Nc5ccc(F)cc5)CC4)cc3F)ccnc2cc1OCCCOCCCOCCC(=O)NCCOc1cccc2c1C(=O)N(C1CCC(=O)NC1=O)C2=O ; NCCOc1cccc2c1C(=O)N(C1CCC(=O)NC1=O)C2=O ; COc1cc2c(Oc3ccc(NC(=O)C4(C(=O)Nc5ccc(F)cc5)CC4)cc3F)ccnc2cc1OCCCCOCCCCOCCC(=O)NCCOc1cccc2c1C(=O)N(C1CCC(=O)NC1=O)C2=O]

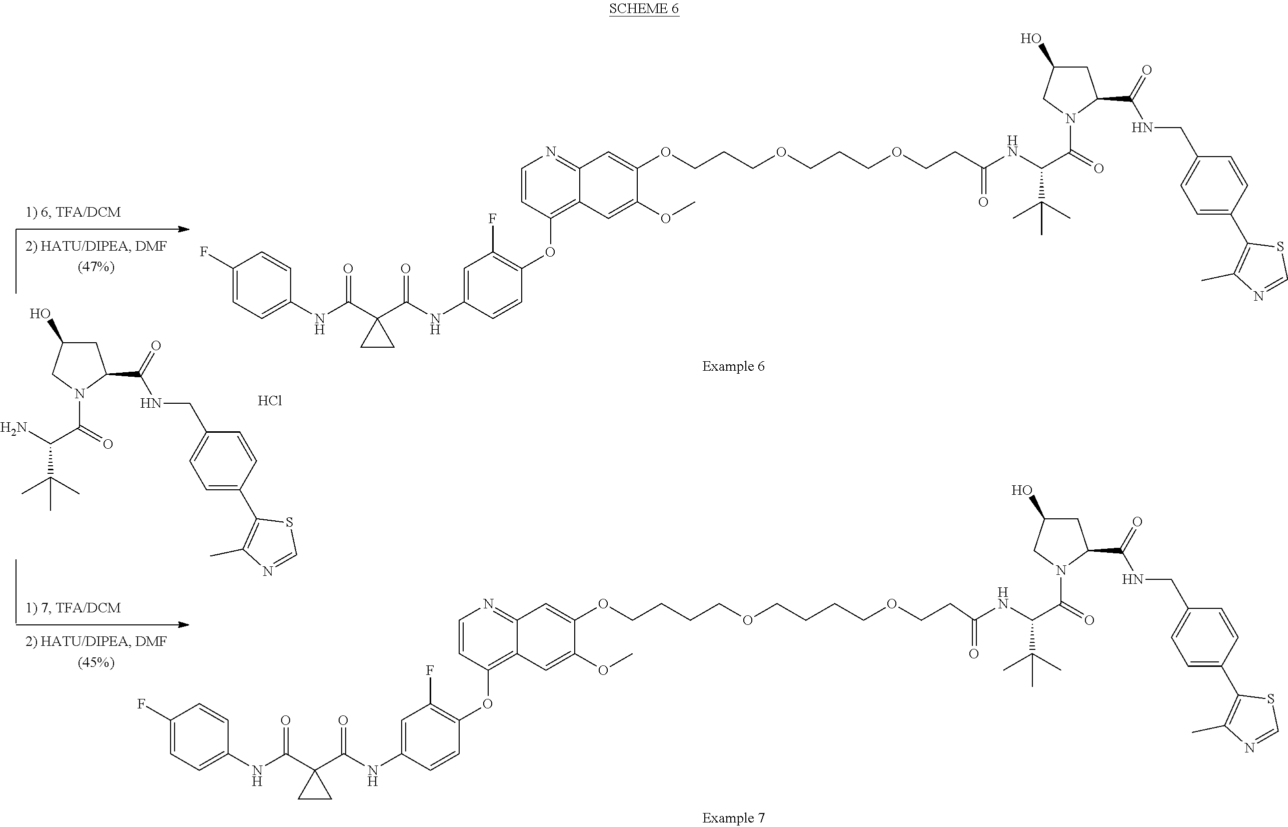 [SMILES: Cc1ncsc1-c1ccc(CNC(=O)[C@@H]2C[C@H](O)CN2C(=O)[C@@H](N)C(C)(C)C)cc1 ; COc1cc2c(Oc3ccc(NC(=O)C4(C(=O)Nc5ccc(F)cc5)CC4)cc3F)ccnc2cc1OCCCCOCCCCOCCC(=O)N[C@H](C(=O)N1C[C@@H](O)C[C@H]1C(=O)NCc1ccc(-c2scnc2C)cc1)C(C)(C)C ; COc1cc2c(Oc3ccc(NC(=O)C4(C(=O)Nc5ccc(F)cc5)CC4)cc3F)ccnc2cc1OCCCOCCCOCCC(=O)N[C@H](C(=O)N1C[C@@H](O)C[C@H]1C(=O)NCc1ccc(-c2scnc2C)cc1)C(C)(C)C ; Cl]

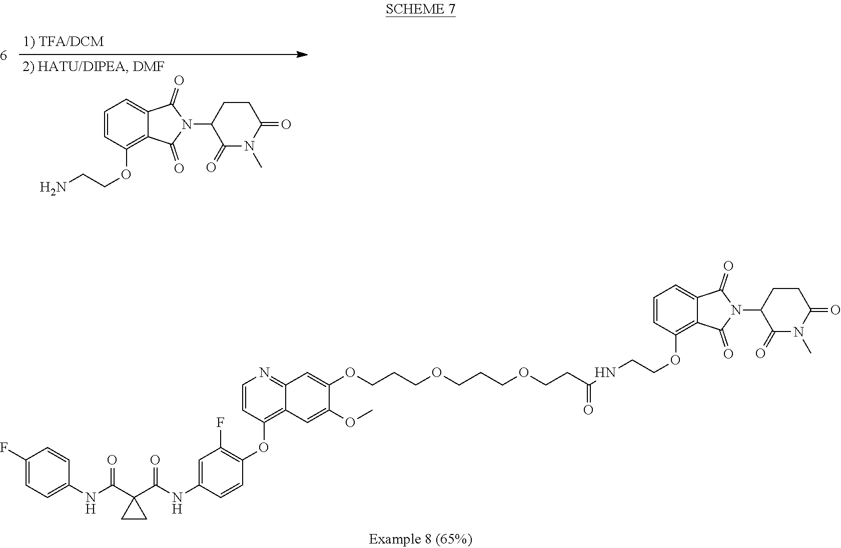 [SMILES: CN1C(=O)CCC(N2C(=O)c3cccc(OCCN)c3C2=O)C1=O ; COc1cc2c(Oc3ccc(NC(=O)C4(C(=O)Nc5ccc(F)cc5)CC4)cc3F)ccnc2cc1OCCCOCCCOCCC(=O)NCCOc1cccc2c1C(=O)N(C1CCC(=O)N(C)C1=O)C2=O]

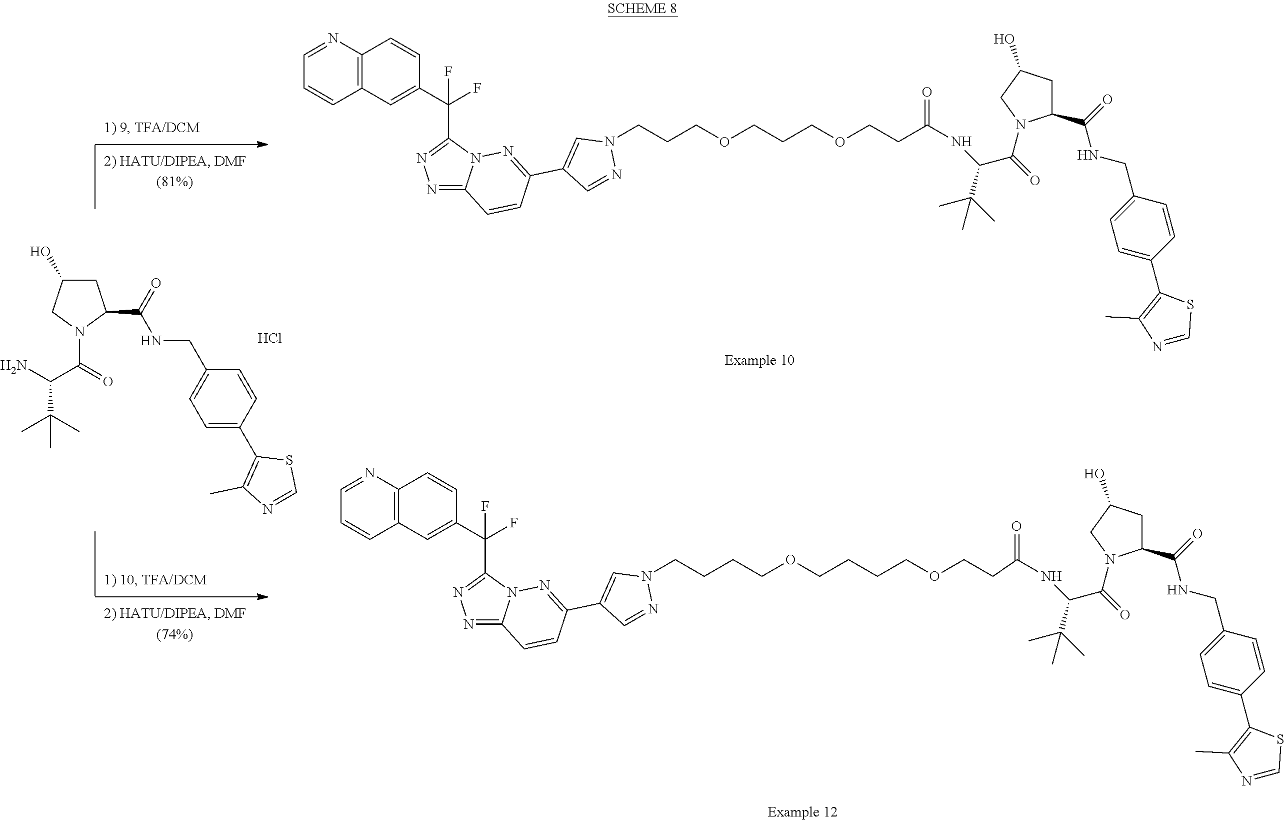

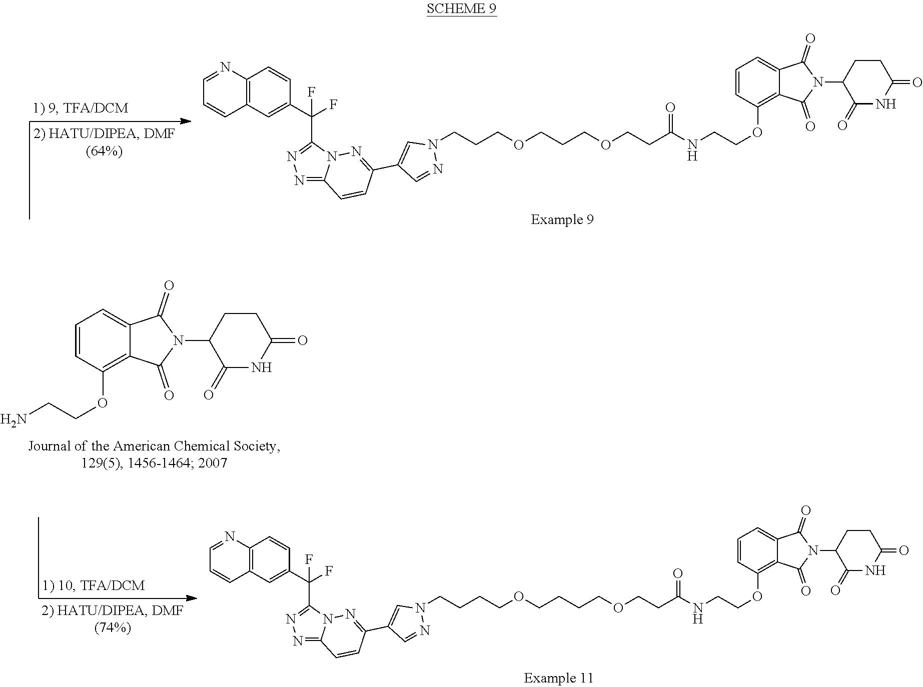 [SMILES: O=C(CCOCCCOCCCn1cc(-c2ccc3nnc(C(F)(F)c4ccc5ncccc5c4)n3n2)cn1)NCCOc1cccc2c1C(=O)N(C1CCC(=O)NC1=O)C2=O ; NCCOc1cccc2c1C(=O)N(C1CCC(=O)NC1=O)C2=O ; O=C(CCOCCCCOCCCCn1cc(-c2ccc3nnc(C(F)(F)c4ccc5ncccc5c4)n3n2)cn1)NCCOc1cccc2c1C(=O)N(C1CCC(=O)NC1=O)C2=O]

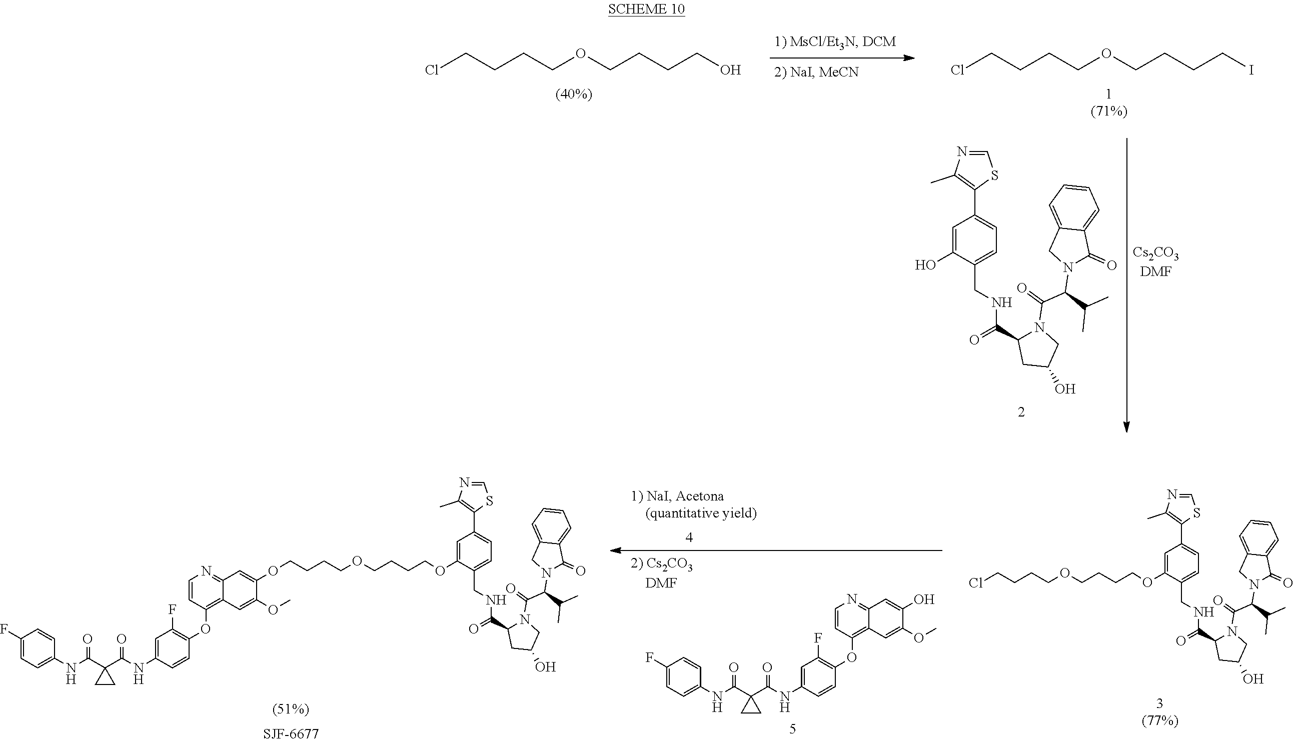 [SMILES: OCCCCOCCCCCl ; ClCCCCOCCCCI ; Cc1ncsc1-c1ccc(CNC(=O)[C@@H]2C[C@@H](O)CN2C(=O)[C@H](C(C)C)N2Cc3ccccc3C2=O)c(O)c1 ; COc1cc2c(Oc3ccc(NC(=O)C4(C(=O)Nc5ccc(F)cc5)CC4)cc3F)ccnc2cc1OCCCCOCCCCOc1cc(-c2scnc2C)ccc1CNC(=O)[C@@H]1C[C@@H](O)CN1C(=O)[C@H](C(C)C)N1Cc2ccccc2C1=O ; COc1cc2c(Oc3ccc(NC(=O)C4(C(=O)Nc5ccc(F)cc5)CC4)cc3F)ccnc2cc1O ; Cc1ncsc1-c1ccc(CNC(=O)[C@@H]2C[C@@H](O)CN2C(=O)[C@H](C(C)C)N2Cc3ccccc3C2=O)c(OCCCCOCCCCCl)c1]

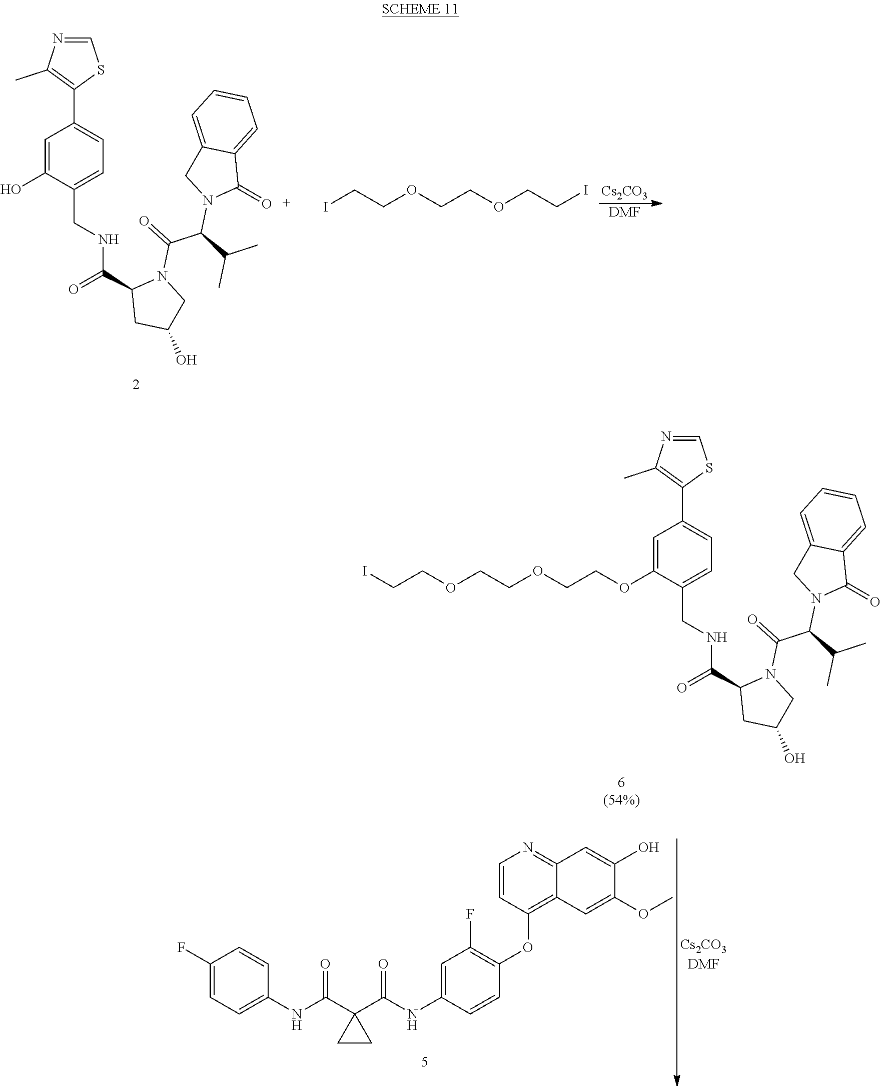

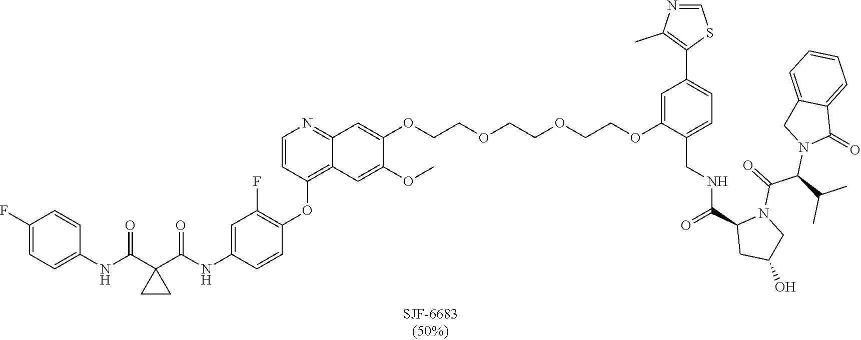 [SMILES: COc1cc2c(Oc3ccc(NC(=O)C4(C(=O)Nc5ccc(F)cc5)CC4)cc3F)ccnc2cc1OCCOCCOCCOc1cc(-c2scnc2C)ccc1CNC(=O)[C@@H]1C[C@@H](O)CN1C(=O)[C@H](C(C)C)N1Cc2ccccc2C1=O]

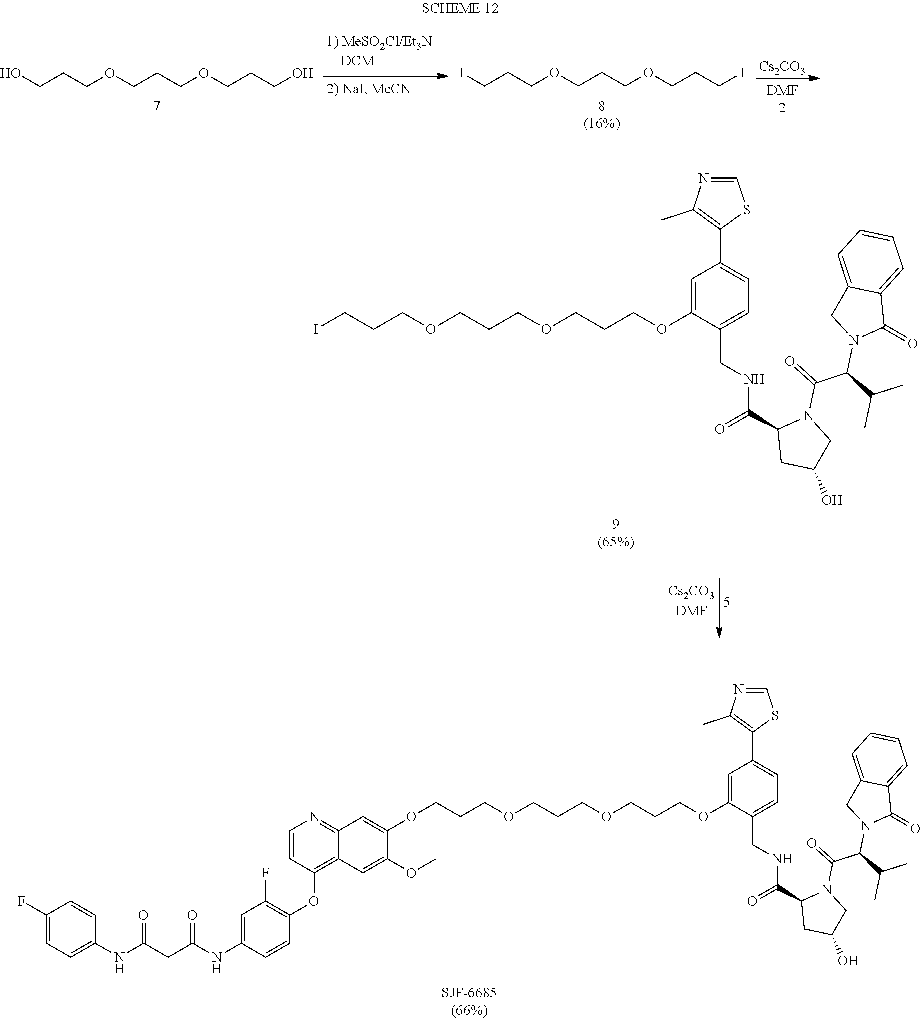

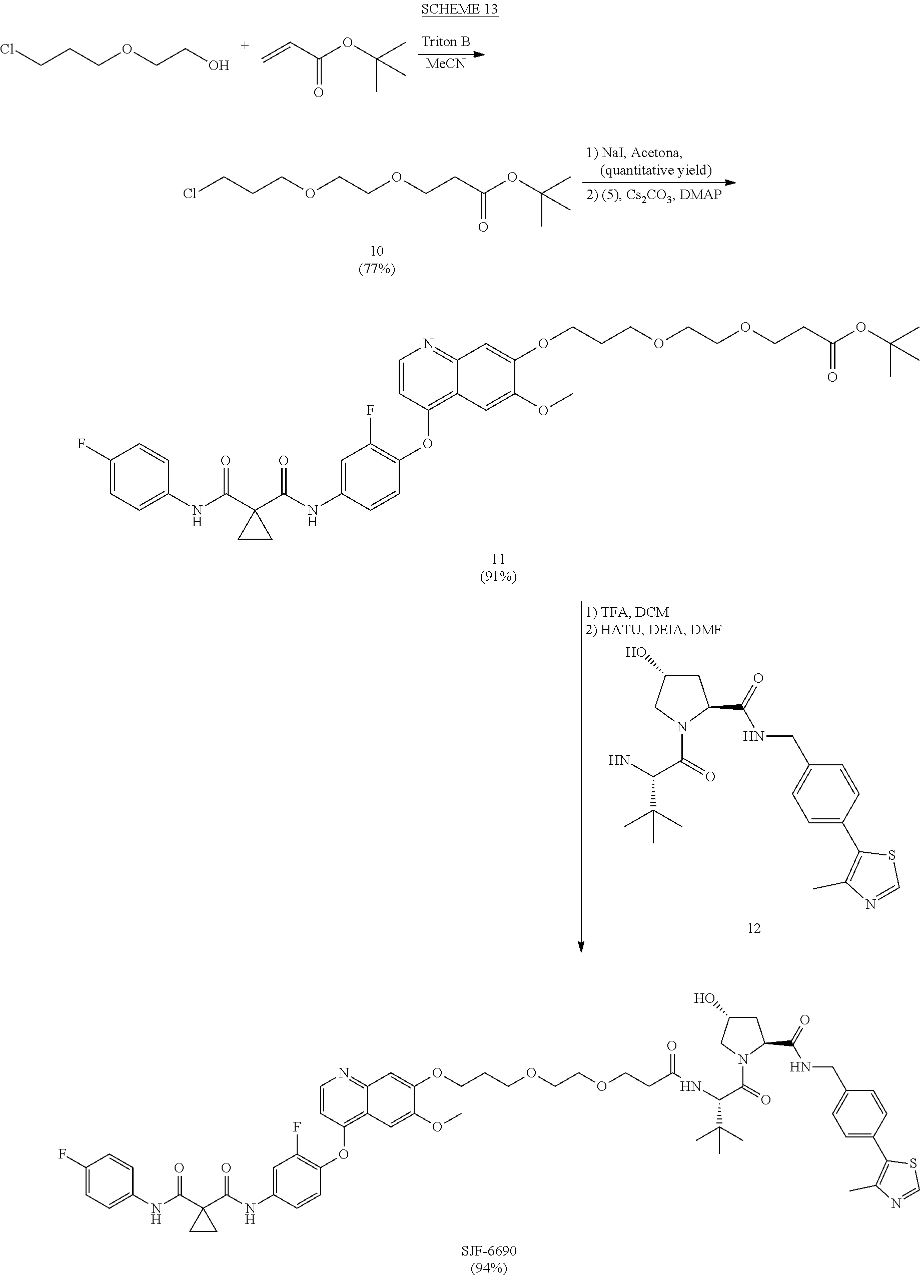

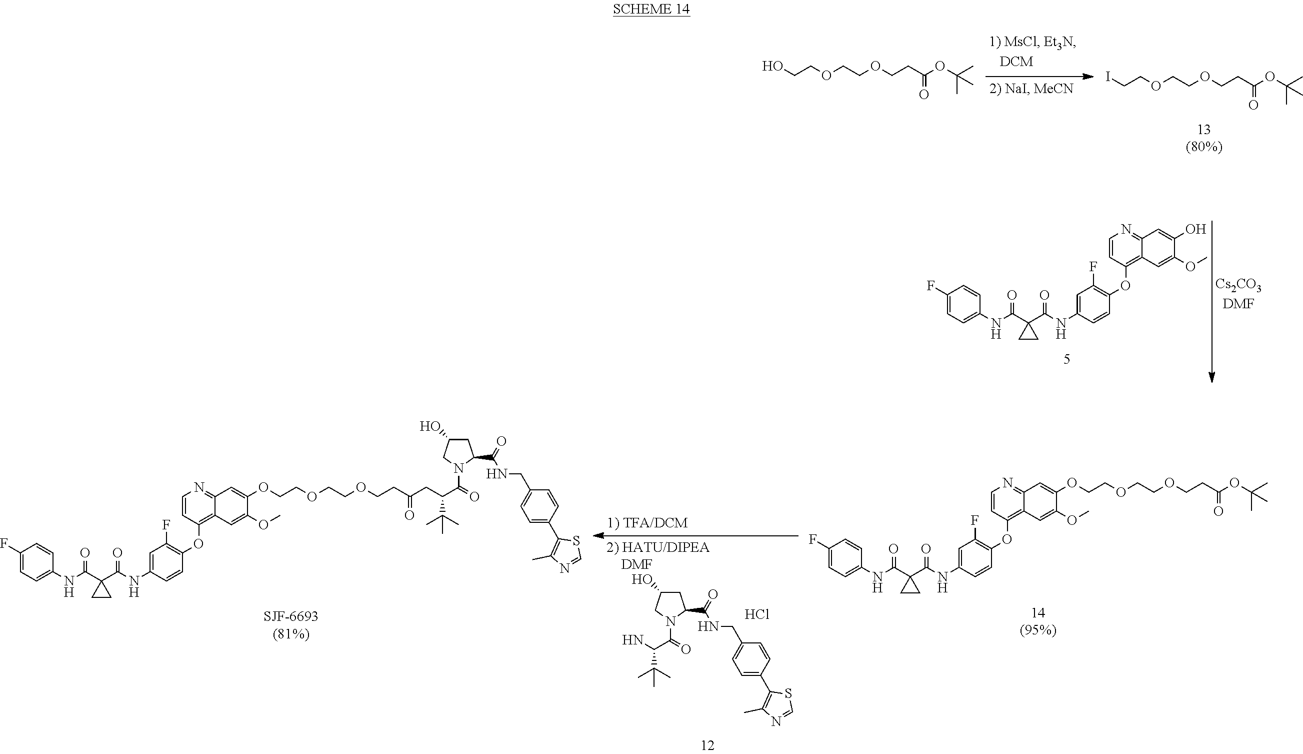

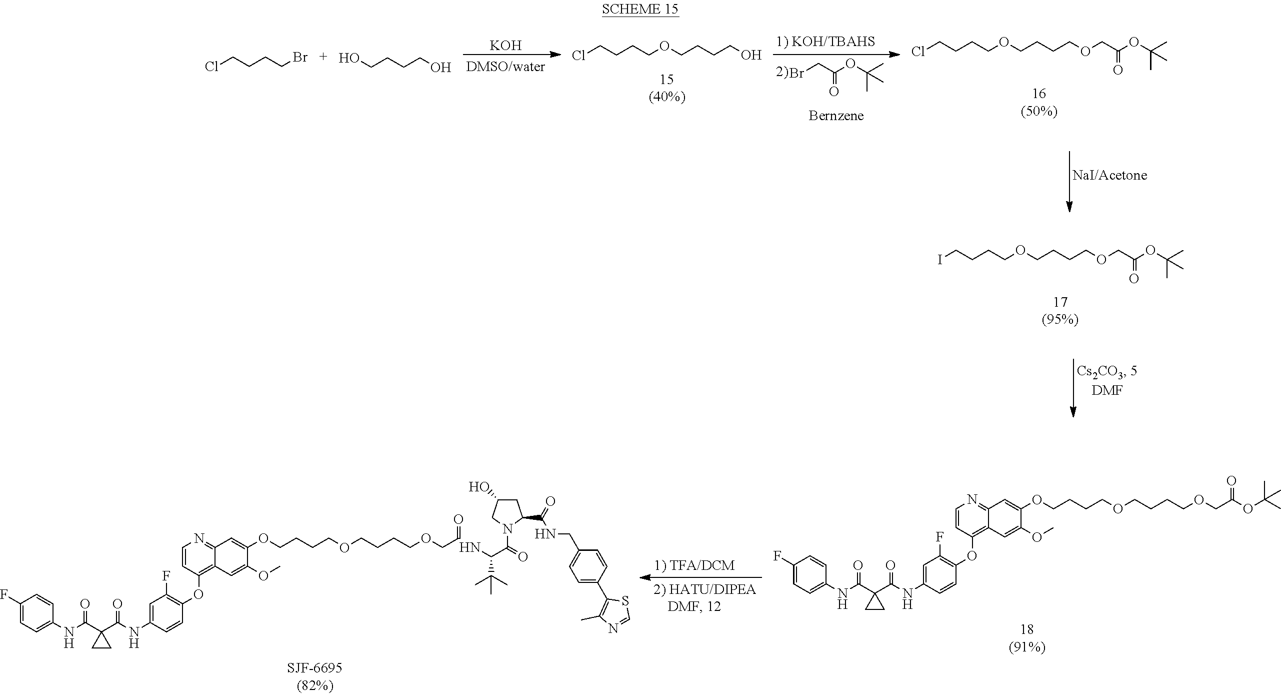

### Protein Level Control

This description also provides methods for the control of protein levels with a cell. This is based on the use of compounds as described herein, which are known to interact with a specific target protein such that degradation of a target protein in vivo will result in the control of the amount of protein in a biological system, preferably to a particular therapeutic benefit.

The following examples are used to assist in describing the present invention, but should not be seen as limiting the present invention in any way.

### Specific Embodiments of the Present Disclosure

The present disclosure encompasses the following specific embodiments. These following embodiments may include all of the features recited in a proceeding embodiment, as specified. Where applicable, the following embodiments may also include the features recited in any proceeding embodiment or aspect inclusively or in the alternative (e.g., an eighth embodiment may include the features recited in a first embodiment, as recited, and/or the features of any of the second through seventh embodiments).

According to an aspect, the present disclosure provides a bifunctional compound having the chemical structure:

PTM-L-ULM,

or a pharmaceutically acceptable salt, enantiomer, stereoisomer, solvate, polymorph or prodrug thereof,

wherein:
the ULM is a small molecule E3 ubiquitin ligase binding moiety that binds an E3 ubiquitin ligase;
the L is a bond or a chemical linking moiety connecting the ULM and the PTM; and
the PTM is selected from:

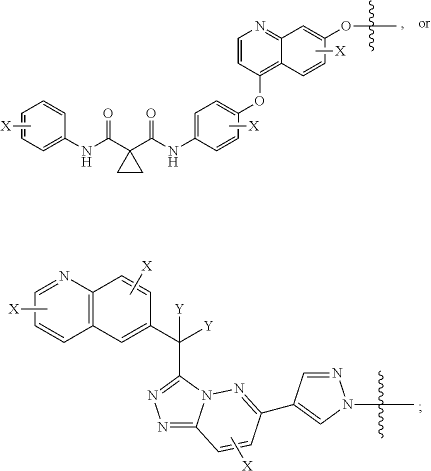

each X is independently Cl, F, Br, H, CN, Me, OMe, or OCF₃ and;

each Y is independently F or H.

In any aspect or embodiment described herein, the E3 ubiquitin ligase binding moiety that targets an E3 ubiquitin ligase selected from the group consisting of Von Hippel-Lindau (VLM), cereblon (CLM), mouse double-minute homolog2 (MLM), and IAP (ILM).

In any aspect or embodiment described herein, the ULM is a Von Hippel-Lindau (VHL) ligase-binding moiety (VLM) with a chemical structure represented by:

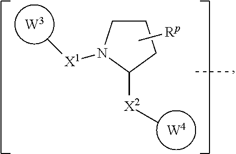

wherein:

X¹, X² are each independently selected from the group of a bond, O, NR^{Y3}, CR^{Y3}R^{Y4}, C═O, C═S, SO, and SO₂;

R^{Y3}, R^{Y4} are each independently selected from the group of H, linear or branched C₁₋₆ alkyl, optionally substituted by 1 or more halo, optionally substituted C₁₋₆ alkoxyl (e.g., optionally substituted by 0-3 R^{P} groups);

R^{P} is 0, 1, 2, or 3 groups, each independently selected from the group H, halo, —OH, C₁₋₃ alkyl, C═O;

W³ is selected from the group of an optionally substituted T, an optionally substituted -T-N(R^{1a}R^{1b})X³, optionally substituted -T-N(R^{1a}R^{1b}), optionally substituted -T-Aryl, an optionally substituted -T-Heteroaryl, an optionally substituted T-biheteroaryl, an optionally substituted -T-Heterocycle, an optionally substituted -T-biheterocycle, an optionally substituted —NR¹-T-Aryl, an optionally substituted —NR¹-T-Heteroaryl or an optionally substituted —NR¹-T-Heterocycle;

X³ is C═O, R¹, R^{1a}, R^{1b};

each of R¹, R^{1a}, R^{1b} independently selected from the group consisting of H, linear or branched C₁-C₆ alkyl group optionally substituted by 1 or more halo or —OH groups, R^{Y3}C═O, R^{Y3}C═S, R^{Y3}SO, R^{Y3}SO₂, N(R^{Y3}R^{Y4})C═O, N(R^{Y3}R^{Y4})C═S, N(R^{Y3}R^{Y4})SO, and N(R^{Y3}R^{Y4})SO₂;

T is selected from the group of an optionally substituted alkyl, —(CH₂)ₙ— group, wherein each one of the methylene groups is optionally substituted with one or two substituents selected from the group of halogen, methyl, optionally substituted alkoxy, a linear or branched C₁-C₆alkyl group optionally substituted by 1 or more halogen, C(O) NR¹R^{1a}, or NR¹R^{1a} or R¹ and R^{1a} are joined to form an optionally substituted heterocycle, or —OH groups or an amino acid side chain optionally substituted; and

n is 0 to 6,

W4 is

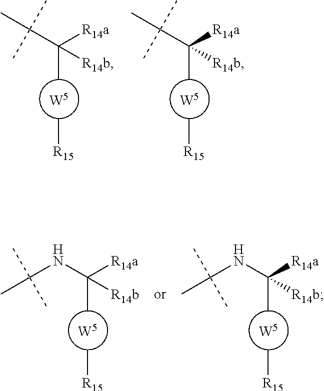

R₁₄ₐ, R_{14b}, are each independently selected from the group of H, haloalkyl, or optionally substituted alkyl;

W⁵ is selected from the group of a phenyl or a 5-10 membered heteroaryl,

R₁₅ is selected from the group of H, halogen, CN, OH, NO₂, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐ SO₂R_{14b}, optionally substituted alkyl, optionally substituted haloalkyl, optionally substituted haloalkoxy; optionally substituted aryl, optionally substituted heteroaryl, optionally substituted cycloalkyl, or optionally substituted cycloheteroalkyl;

and wherein the dashed line indicates the site of attachment of at least one PTM, another ULM (ULM′) or a chemical linker moiety coupling at least one PTM or a ULM′ or both to ULM.

In any aspect or embodiment described herein, the ULM is a Von Hippel-Lindau (VHL) ligase-binding moiety (VLM) with a chemical structure represented by:

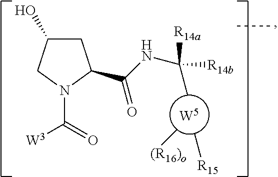

wherein:
W³ is selected from the group of an optionally substituted aryl, optionally substituted heteroaryl, or

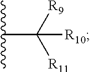

R₉ and R₁₀ are independently hydrogen, optionally substituted alkyl, optionally substituted cycloalkyl, optionally substituted hydroxyalkyl, optionally substituted heteroaryl, or haloalkyl, or R₉, R₁₀, and the carbon atom to which they are attached form an optionally substituted cycloalkyl;
R₁₁ is selected from the group of an optionally substituted heterocyclic, optionally substituted alkoxy, optionally substituted heteroaryl, optionally substituted aryl,

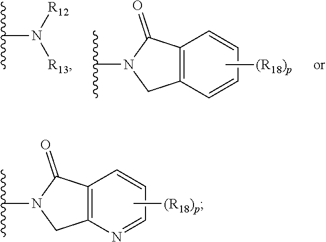

R₁₂ is selected from the group of H or optionally substituted alkyl;
R₁₃ is selected from the group of H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R₁₄ₐ, R_{14b}, are each independently selected from the group of H, haloalkyl, or optionally substituted alkyl;
W⁵ is selected from the group of a phenyl or a 5-10 membered heteroaryl,
R₁₅ is selected from the group of H, halogen, CN, OH, NO₂, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐ SO₂R_{14b}, optionally substituted alkyl, optionally substituted haloalkyl, optionally substituted haloalkoxy; optionally substituted aryl; optionally substituted heteroaryl; optionally substituted cycloalkyl; or optionally substituted cycloheteroalkyl;
R₁₆ is independently selected from the group of halo, optionally substituted alkyl, optionally substituted haloalkyl, hydroxy, or optionally substituted haloalkoxy;
o is 0, 1, 2, 3, or 4;
R₁₈ is independently selected from the group of H, halo, optionally substituted alkoxy, cyano, optionally substituted alkyl, haloalkyl, haloalkoxy or a linker; and
p is 0, 1, 2, 3, or 4, and wherein the dashed line indicates the site of attachment of at least one PTM, another ULM (ULM′) or a chemical linker moiety coupling at least one PTM or a ULM′ or both to ULM.

In any aspect or embodiment described herein, the ULM has a chemical structure selected from the group of:

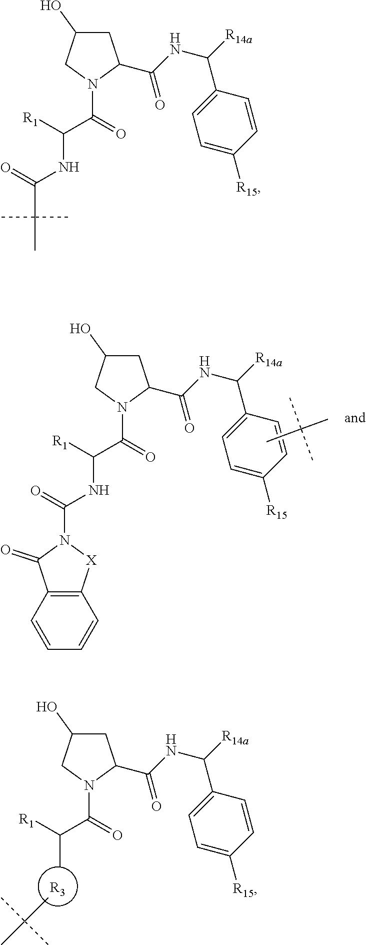

wherein:
R₁ is H, ethyl, isopropyl, tert-butyl, sec-butyl, cyclopropyl, cyclobutyl, cyclopentyl, or cyclohexyl; optionally substituted alkyl, optionally substituted hydroxyalkyl, optionally substituted heteroaryl, or haloalkyl;
R₁₄ₐ is H, haloalkyl, optionally substituted alkyl, methyl, fluoromethyl, hydroxymethyl, ethyl, isopropyl, or cyclopropyl;
R₁₅ is selected from the group consisting of H, halogen, CN, OH, NO₂, optionally substituted heteroaryl, optionally substituted aryl; optionally substituted alkyl, optionally substituted haloalkyl, optionally substituted haloalkoxy, optionally substituted cycloalkyl, or optionally substituted cycloheteroalkyl;
X is C, CH₂, or C═O;
R₃ is absent or an optionally substituted 5 or 6 membered heteroaryl; and
wherein the dashed line indicates the site of attachment of at least one PTM, another ULM (ULM′) or a chemical linker moiety coupling at least one PTM or a ULM′ or both to the ULM.

In any aspect or embodiment described herein, the ULM comprises a group according to the chemical structure:

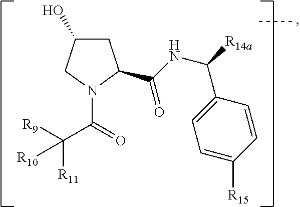

wherein:
R₁₄ₐ is H, haloalkyl, optionally substituted alkyl, methyl, fluoromethyl, hydroxymethyl, ethyl, isopropyl, or cyclopropyl;
R9 is H;
R10 is H, ethyl, isopropyl, tert-butyl, sec-butyl, cyclopropyl, cyclobutyl, cyclopentyl, or cyclohexyl;
R11 is

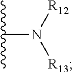

optionally substituted heteroaryl,

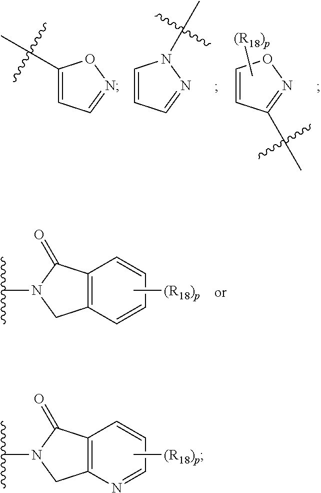

p is 0, 1, 2, 3, or 4; and
each R₁₈ is independently halo, optionally substituted alkoxy, cyano, optionally substituted alkyl, haloalkyl, haloalkoxy or a linker;
R12 is H, C═O
R13 is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl,
R₁₅ is selected from the group consisting of H, halogen, Cl, CN, OH, NO₂, optionally substituted heteroaryl, optionally substituted aryl;

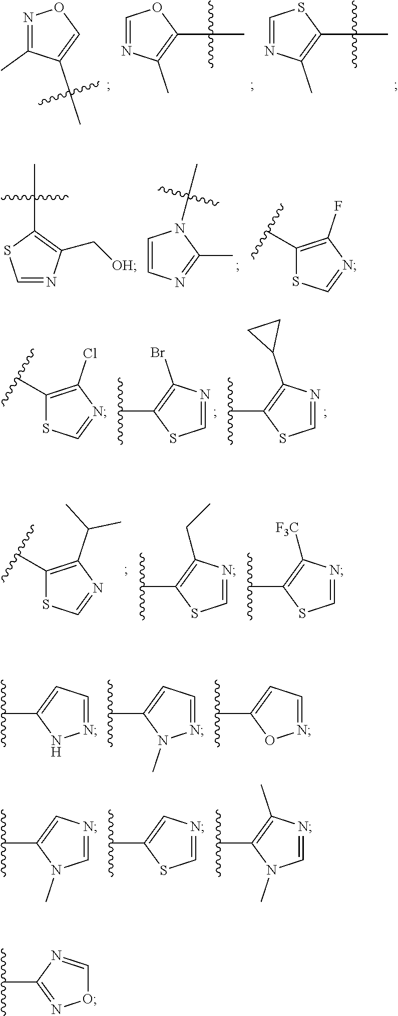

and wherein the dashed line indicates the site of attachment of at least one PTM, another ULM (ULM′) or a chemical linker moiety coupling at least one PTM or a ULM′ or both to the ULM.

In any aspect or embodiment described herein, the ULM comprises a group according to the chemical structure:

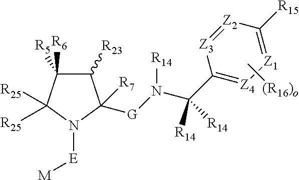

wherein:
each R₅ and R₆ is independently —OH, —SH, or optionally substituted alkyl or R₅, R₆, and the carbon atom to which they are attached form a carbonyl;
R₇ is H or optionally substituted alkyl;
E is a bond, C═O, or C═S;
G is a bond, optionally substituted alkyl, —COOH or C=J;
J is O or N—R₈;
R₈ is H, CN, optionally substituted alkyl or optionally substituted alkoxy;
M is optionally substituted aryl, optionally substituted heteroaryl, optionally substituted heterocyclic or

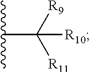

each R₉ and R₁₀ is independently H; optionally substituted alkyl, optionally substituted cycloalkyl, optionally substituted hydroxyalkyl, optionally substituted thioalkyl, a disulphide linked ULM, optionally substituted heteroaryl, or haloalkyl; or R₉, R₁₀, and the carbon atom to which they are attached form an optionally substituted cycloalkyl;
R₁₁ is optionally substituted heterocyclic, optionally substituted alkoxy, optionally substituted heteroaryl, optionally substituted aryl, or

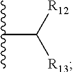

R₁₂ is H or optionally substituted alkyl;
R₁₃ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl; optionally substituted (oxoalkyl)carbamate,
each R₁₄ is independently H, haloalkyl, optionally substituted cycloalkyl, optionally substituted alkyl or optionally substituted heterocycloalkyl;
R₁₅ is H, optionally substituted heteroaryl, haloalkyl, optionally substituted aryl, optionally substituted alkoxy, or optionally substituted heterocyclyl;
each R₁₆ is independently halo, optionally substituted alkyl, optionally substituted haloalkyl, CN, or optionally substituted haloalkoxy;
each R₂₅ is independently H or optionally substituted alkyl; or both R₂₅ groups can be taken together to form an oxo or optionally substituted cycloalkyl group;
R₂₃ is H or OH;
Z₁, Z₂, Z₃, and Z₄ are independently C or N; and
o is 0, 1, 2, 3, or 4, or a pharmaceutically acceptable salt, stereoisomer, solvate or polymorph thereof.

In any aspect or embodiment described herein, the ULM is a cereblon E3 ligase-binding moiety (CLM) selected from the group consisting of a thalidomide, lenalidomide, pomalidomide, analogs thereof, isosteres thereof, or derivatives thereof.

In any aspect or embodiment described herein, the CLM has a chemical structure represented by:

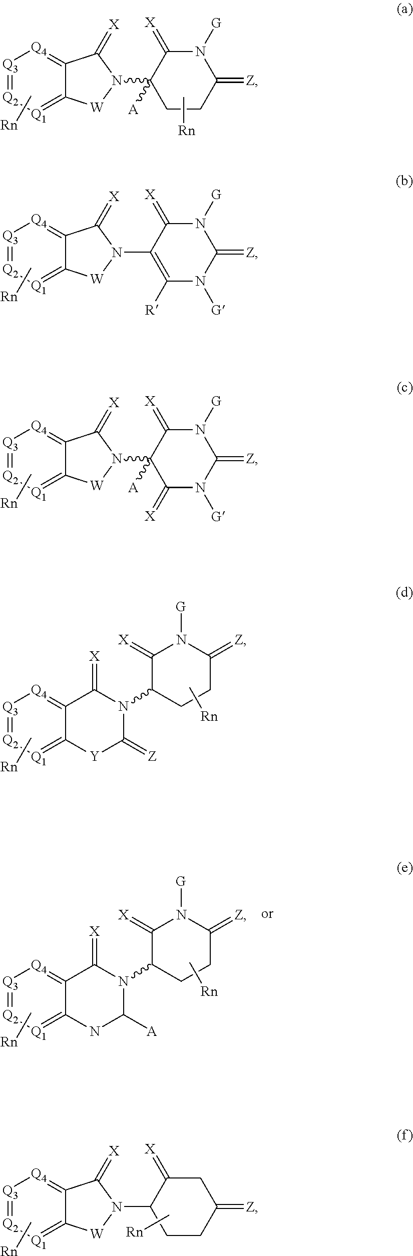

wherein:
W is selected from the group consisting of CH₂, CHR, C═O, SO₂, NH, and N-alkyl;
each X is independently selected from the group consisting of O, S, and H₂,
Y is selected from the group consisting of CH₂, —C═CR′, NH, N-alkyl, N-aryl, N-hetaryl, N-cycloalkyl, N-heterocyclyl, O, and S;
Z is selected from the group consisting of O, S, and H₂;
G and G′ are independently selected from the group consisting of H, optionally substituted linear or branched alkyl, OH, R′ OCOOR, R′ OCONRR″, CH₂-heterocyclyl optionally substituted with R′, and benzyl optionally substituted with R′;
Q₁, Q₂, Q₃, and Q₄ represent a carbon C substituted with a group independently selected from R′, N or N-oxide;
A is independently selected from the group H, optionally substituted linear or branched alkyl, cycloalkyl, Cl and F;
R comprises —CONR′R″, —OR′, —NR′R″, —SR′, —SO₂R′, —SO₂NR′R″, —CR′R″—, —CR′NR′R″—, (—CR′O)_{n′}″, -aryl, -hetaryl, -alkyl, -cycloalkyl, -heterocyclyl, —P(O)(OR′)R″, —P(O)R′R″, —OP(O)(OR′)R″, —OP(O)R′R″, —Cl, —F, —Br, —I, —CF₃, —CN, —NR′SO₂NR′R″, —NR′CONR′R″, —CONR′COR″, —NR′C(═N—CN)NR′R″, —C(═N—CN)NR′R″, —NR′C(═N—CN)R″, —NR′C(═C—NO₂)NR′R″, —SO₂NR′COR″, —NO₂, —CO₂R′, —C(C═N—OR′)R″, —CR′═CR′R″, —CCR′, —S(C═O)(C═N—R′)R″, —SF₅ and —OCF₃;
R′ and R″ are independently selected from the group consisting of a bond, H, alkyl, cycloalkyl, aryl, heteroaryl, heterocyclic, —C(═O)R, heterocyclyl, each of which is optionally substituted;
[symbol] represents a bond that may be stereospecific ((R) or (S)) or non-stereospecific; and
Rₙ comprises from 1 to 4 independently selected functional groups or atoms, for example, O, OH, N, C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy,
wherein n′ is an integer from 1-10, and wherein
when n is 1, Rₙ is modified to be covalently joined to the linker group (L), and
when n is 2, 3, or 4, then one Rₙ is modified to be covalently joined to the linker group (L), and any other Rₙ is optionally modified to be covalently joined to a PTM, a CLM, a second CLM having the same chemical structure as the CLM, a CLM′, a second linker, or any multiple or combination thereof.

In any aspect or embodiment described herein, the CLM has a chemical structure represented by:

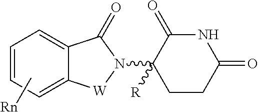

wherein:
W is independently selected from the group CH2, C═O, NH, and N-alkyl;
R is independently selected from a H, methyl, or optionally substituted alkyl (e.g., C1-C6 alkyl (linear, branched, optionally substituted));
[symbol] represents a bond that may be stereospecific ((R) or (S)) or non-stereospecific; and
Rn comprises from 1 to 4 independently selected functional groups or atoms, for example, O, OH, N, C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy, and optionally, one of which is modified to be covalently joined to a PTM, a chemical linker group (L), a CLM (or CLM′) or combination thereof.

In any aspect or embodiment described herein, the ULM is a (MDM2) binding moiety (MLM) with a chemical moiety selected from the group consisting of a substituted imidazolines, a substituted spiro-indolinones, a substituted pyrrolidines, a substituted piperidinones, a substituted morpholinones, a substituted pyrrolopyrimidines, a substituted imidazolopyridines, a substituted thiazoloimidazoline, a substituted pyrrolopyrrolidinones, and a substituted isoquinolinones.

In any aspect or embodiment described herein, the ULM is a IAP E3 ubiquitin ligase binding moiety (ILM) comprising the amino acids alanine (A), valine (V), proline (P), and isoleucine (I) or their unnatural mimetics.

In any aspect or embodiment described herein, the ULM is a IAP E3 ubiquitin ligase binding moiety (ILM) comprising a AVPI tetrapeptide fragment or derivative thereof.

In any aspect or embodiment described herein, the CLM has a chemical structure represented by:

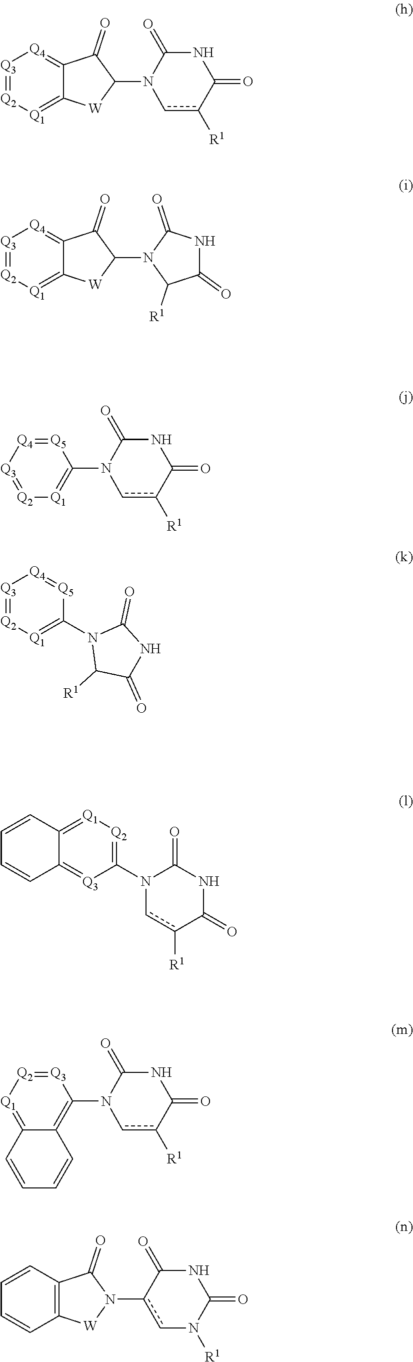

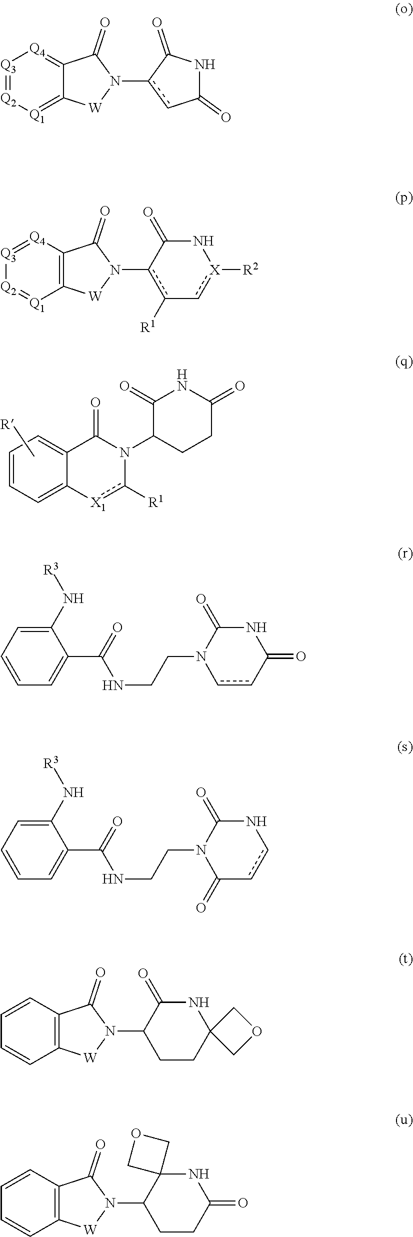

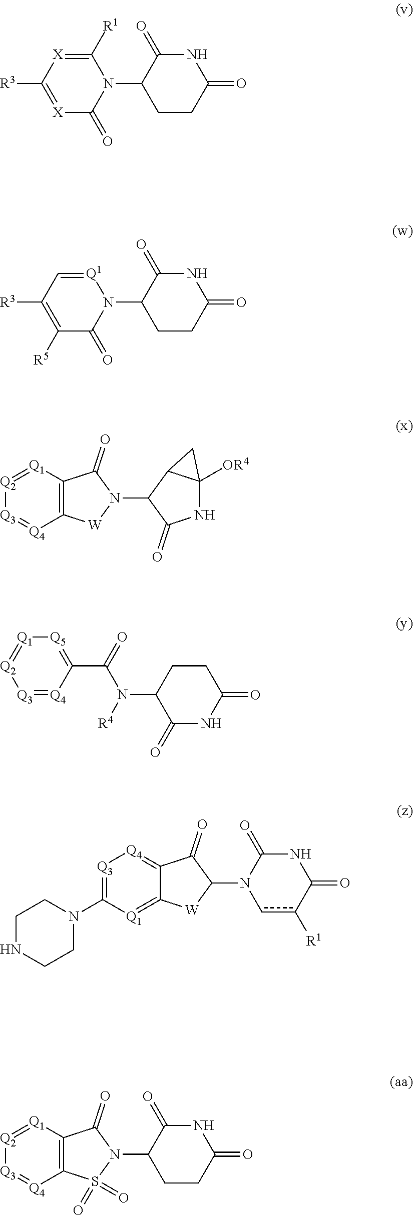

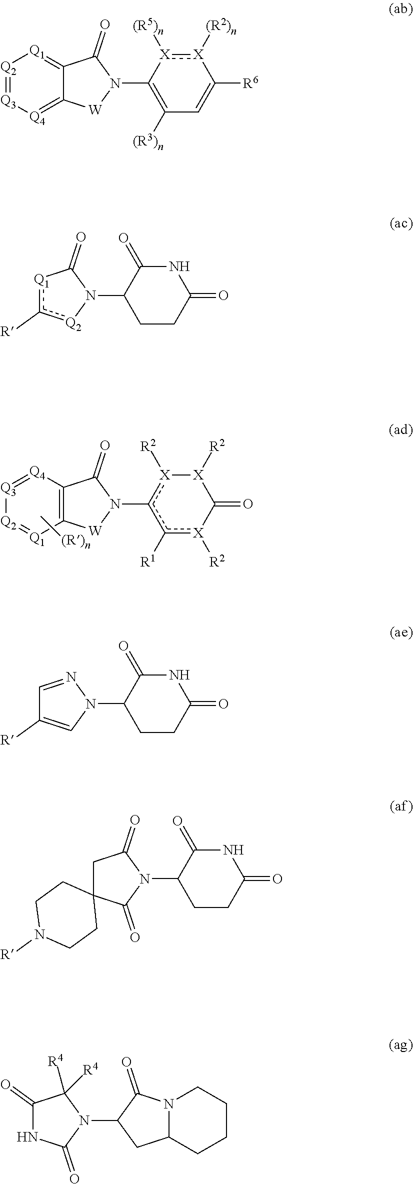

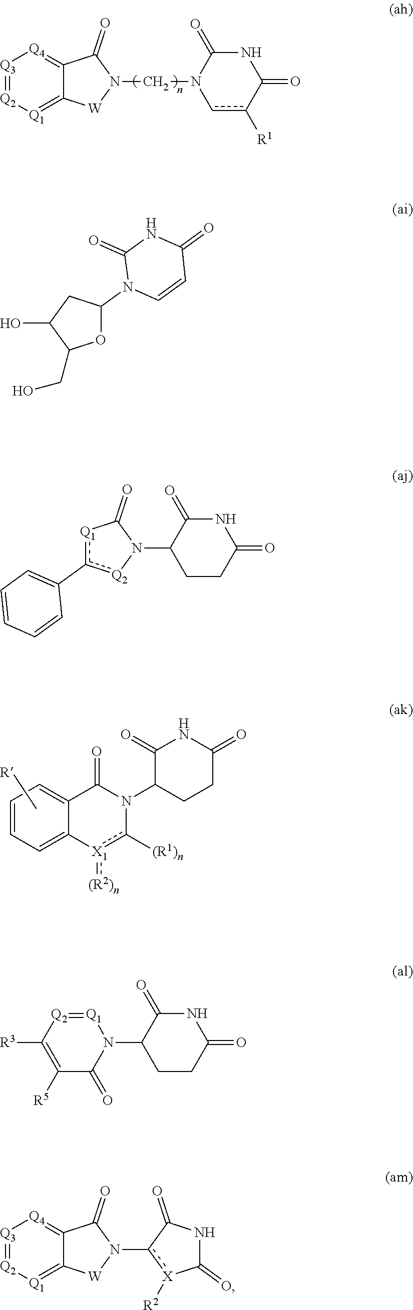

wherein:
W is independently selected from CH₂, CHR, C═O, SO₂, NH, and N-alkyl;
Q₁, Q₂, Q₃, Q₄, Q₅ are each independently represent a carbon C or N substituted with a group independently selected from R′, N or N-oxide;
R¹ is selected from absent, H, OH, CN, C1-C3 alkyl, C═O;
R² is selected from the group absent, H, OH, CN, C1-C3 alkyl, CHF₂, CF₃, CHO, C(═O)NH₂;
R³ is selected from H, alkyl (e.g., C1-C6 or C1-C3 alkyl), substituted alkyl (e.g., substituted C1-C6 or C1-C3 alkyl), alkoxy (e.g., C1-C6 or C1-C3 alkoxyl), substituted alkoxy (e.g., substituted C1-C6 or C1-C3 alkoxyl);
R⁴ is selected from H, alkyl, substituted alkyl;
R⁵ and R⁶ are each independently H, halogen, C(═O)R′, CN, OH, CF₃;
X is C, CH, C═O, or N;
X₁ is C═O, N, CH, or CH₂;
R′ is selected from H, halogen, amine, alkyl (e.g., C1-C3 alkyl), substituted alkyl (e.g., substituted C1-C3 alkyl), alkoxy (e.g., C1-C3 alkoxyl), substituted alkoxy (e.g., substituted C1-C3 alkoxyl), NR²R³, C(═O)OR², optionally substituted phenyl; n is 0-4;
[symbol] is a single or double bond; and
   the CLM is covalently joined to a PTM, a chemical linker group (L), a ULM, CLM (or CLM′) or combination thereof.

In any aspect or embodiment described herein, the linker (L) comprises a chemical structural unit represented by the formula:

-(A)_{q}-, wherein

(A)_{q} is a group which is connected to at least one of ULM moiety, PTM moiety, or both; and

q is an integer greater than or equal to 1;

each A is independently selected from the group consisting of, a bond, CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, siR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₃₋₁₁heteocyclyl optionally substituted with 0-6 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R^{L5} groups; and

R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NH SO₂NH(C₁₋₈alkyl), NH SO₂N(C₁₋₈alkyl)₂, NH SO₂NH₂.

In any aspect or embodiment described herein, the linker (L) comprises a group represented by a general structure selected from the group consisting of:

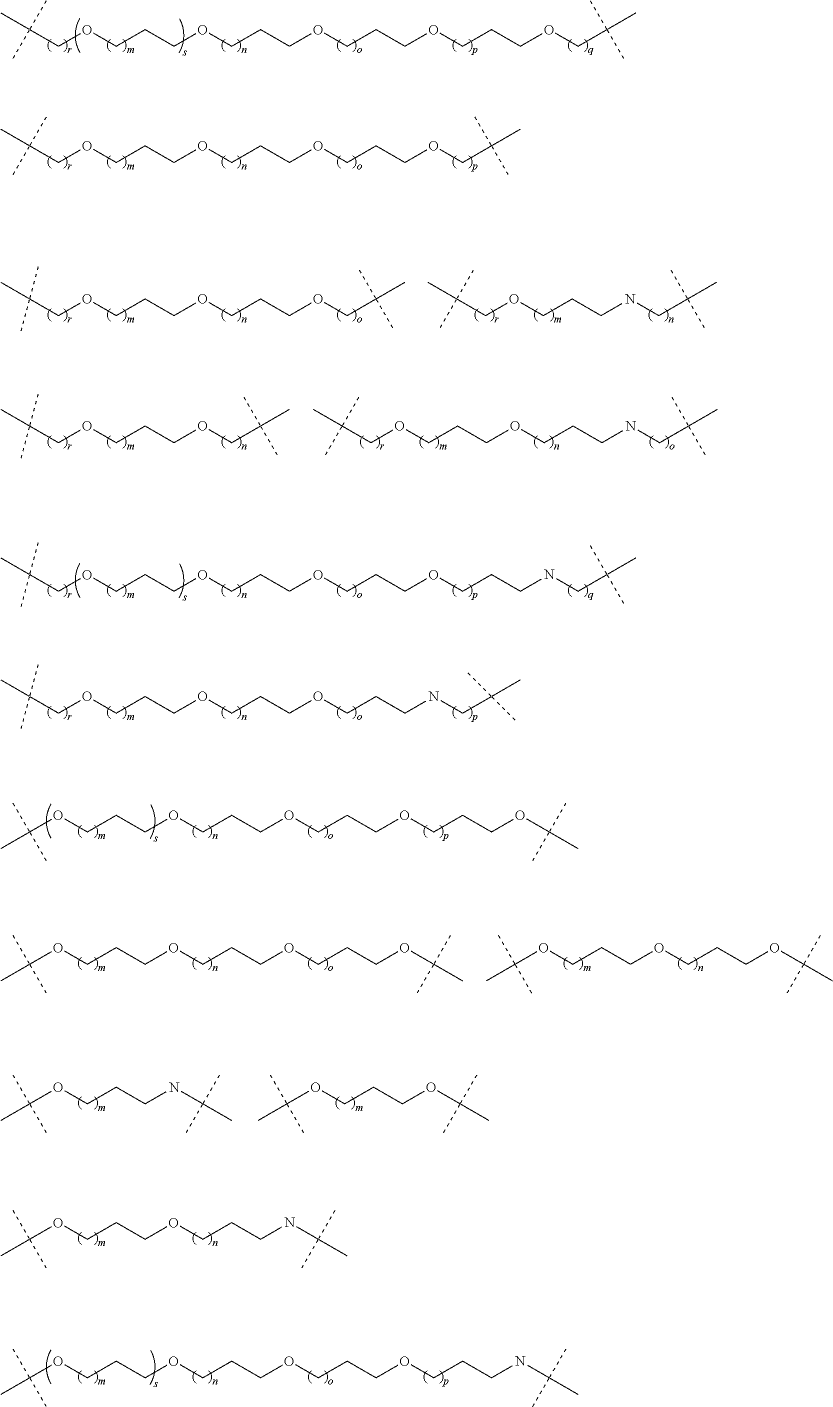

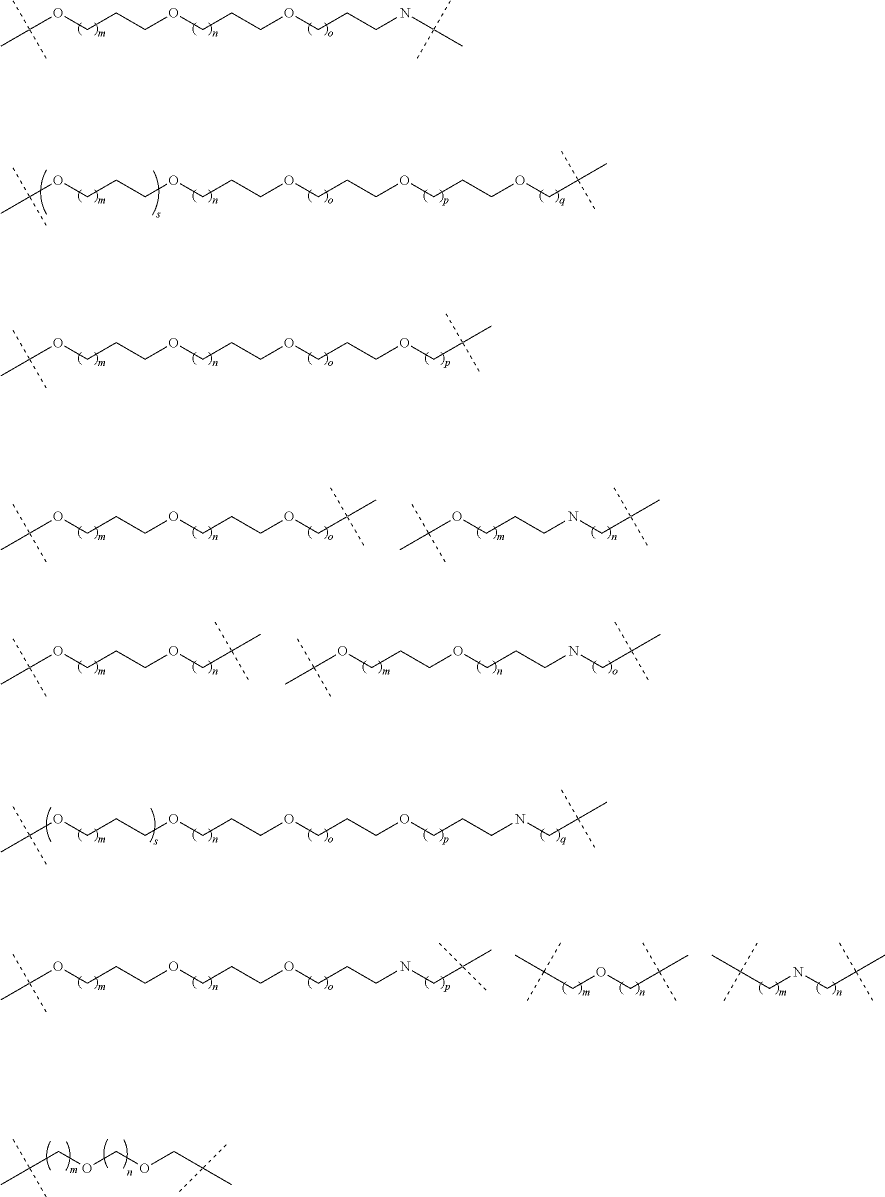

—N(R)—(CH2)ₘ—O(CH2)ₙ—O(CH2)ₒ—O(CH2)ₚ—O(CH2)_{q}—O(CH2)ᵣ-OCH2-, —O—(CH2)ₘ—O(CH2)ₙ—O(CH2)ₒ—O(CH2)ₚ—O(CH2)_{q}—O(CH2)ᵣ-OCH2-, —O—(CH2)ₘ—O(CH2)ₙ—O(CH2)ₒ—O(CH2)ₚ—O(CH2)_{q}—O(CH2)ᵣ—O—;
—N(R)—(CH2)ₘ—O(CH2)ₙ—O(CH2)ₒ—O(CH2)ₚ—O(CH2)_{q}—O(CH2)ᵣ—O—;
—(CH2)ₘ—O(CH2)ₙ—O(CH2)ₒ—O(CH2)ₚ—O(CH2)_{q}—O(CH2)ᵣ—O—;
—(CH2)ₘ—O(CH2)ₙ—O(CH2)ₒ—O(CH2)ₚ—O(CH2)_{q}—O(CH2)ᵣ-OCH2-;

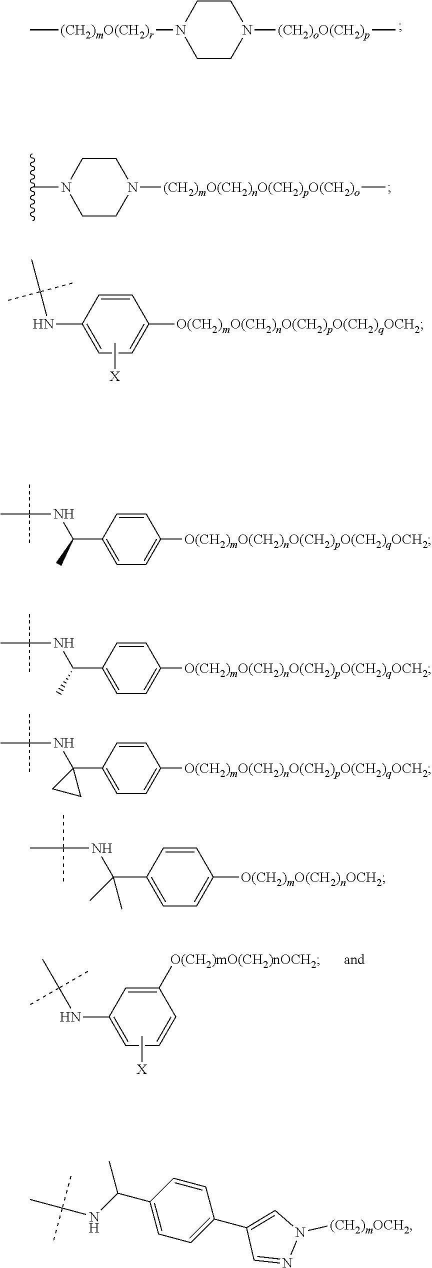

wherein each m, n, o, p, q, r, and s, are independently 0, 1, 2, 3, 4, 5, 6, 7, 8, 9, 10, 11, 12, 13, 14, 15, 16, 17, 18, 19, or 20 with the proviso that when the number is zero, there is no N—O or O—O bond, R is selected from the group H, methyl and ethyl, and X is selected from the group H and F;

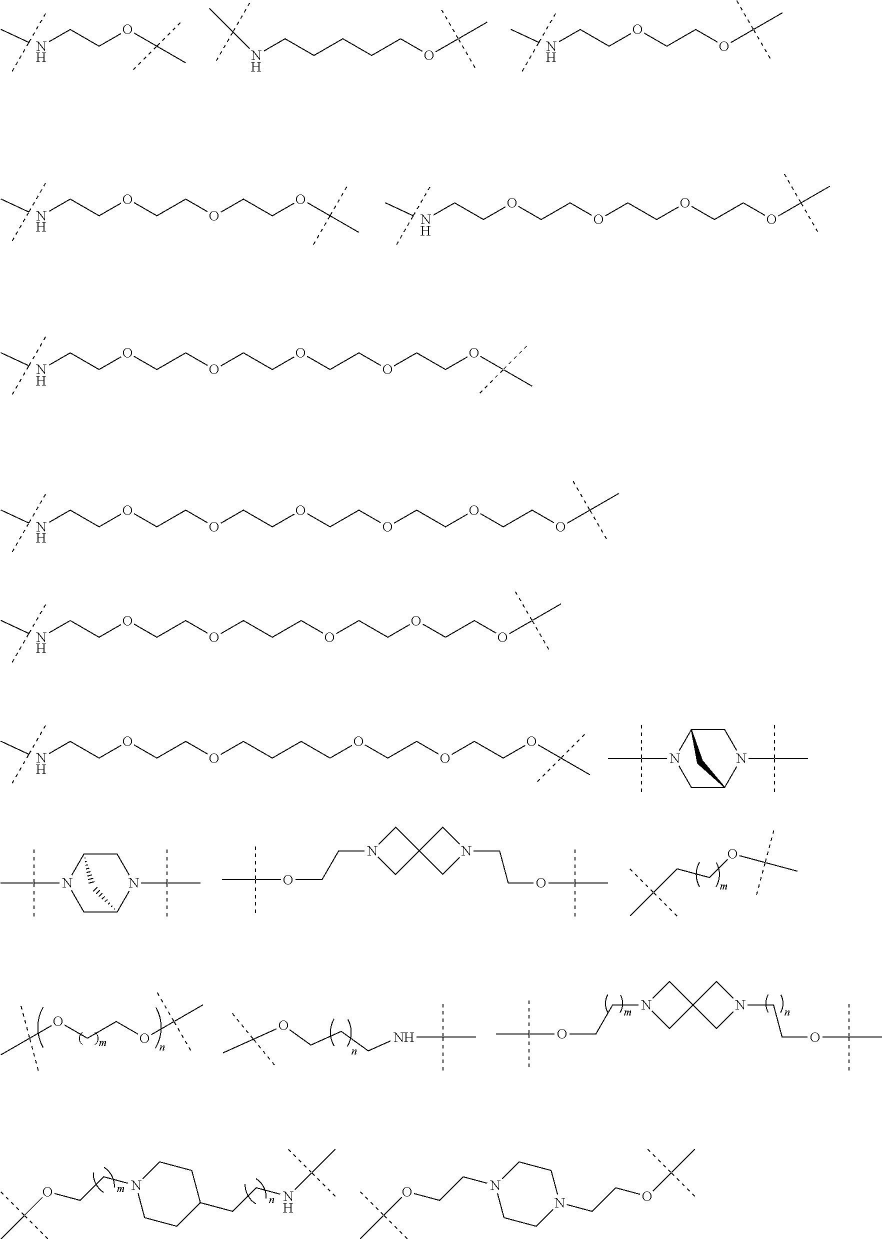

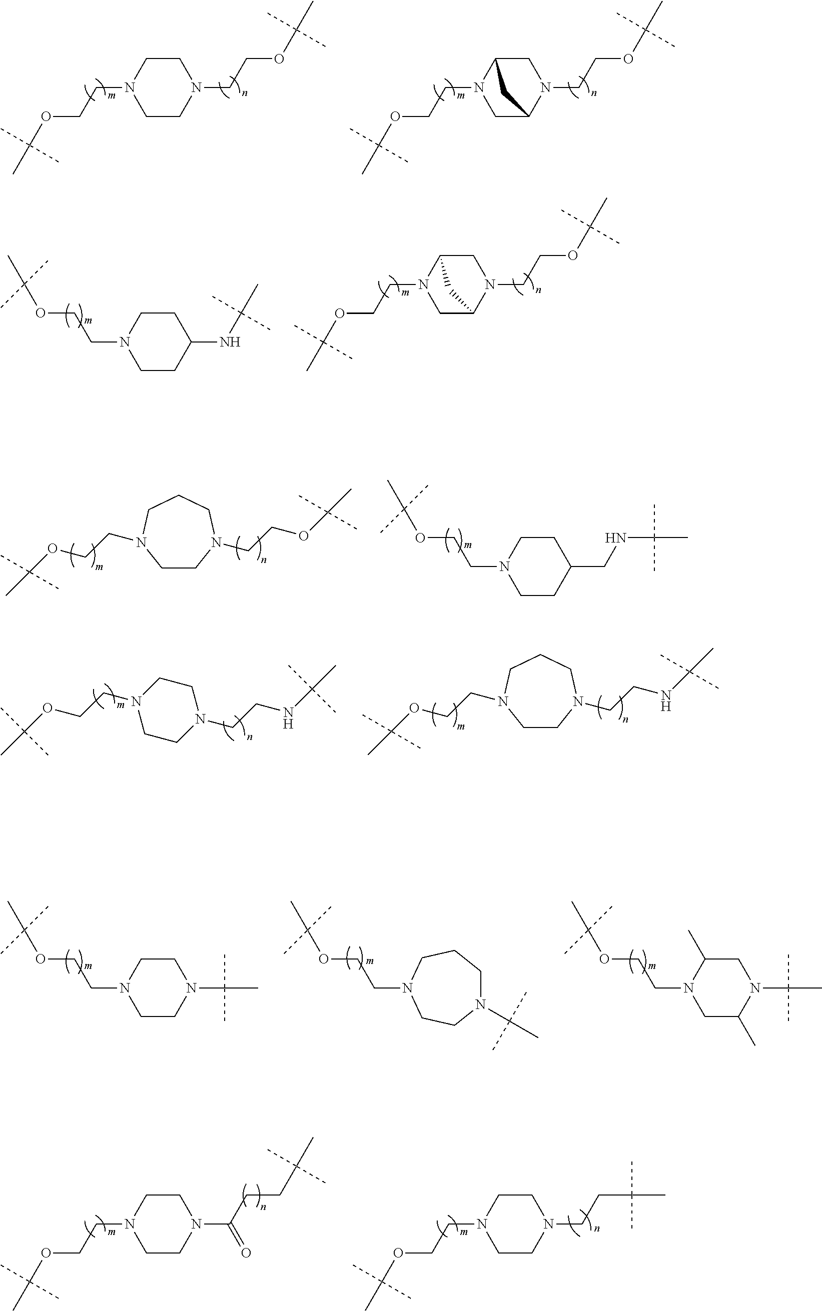

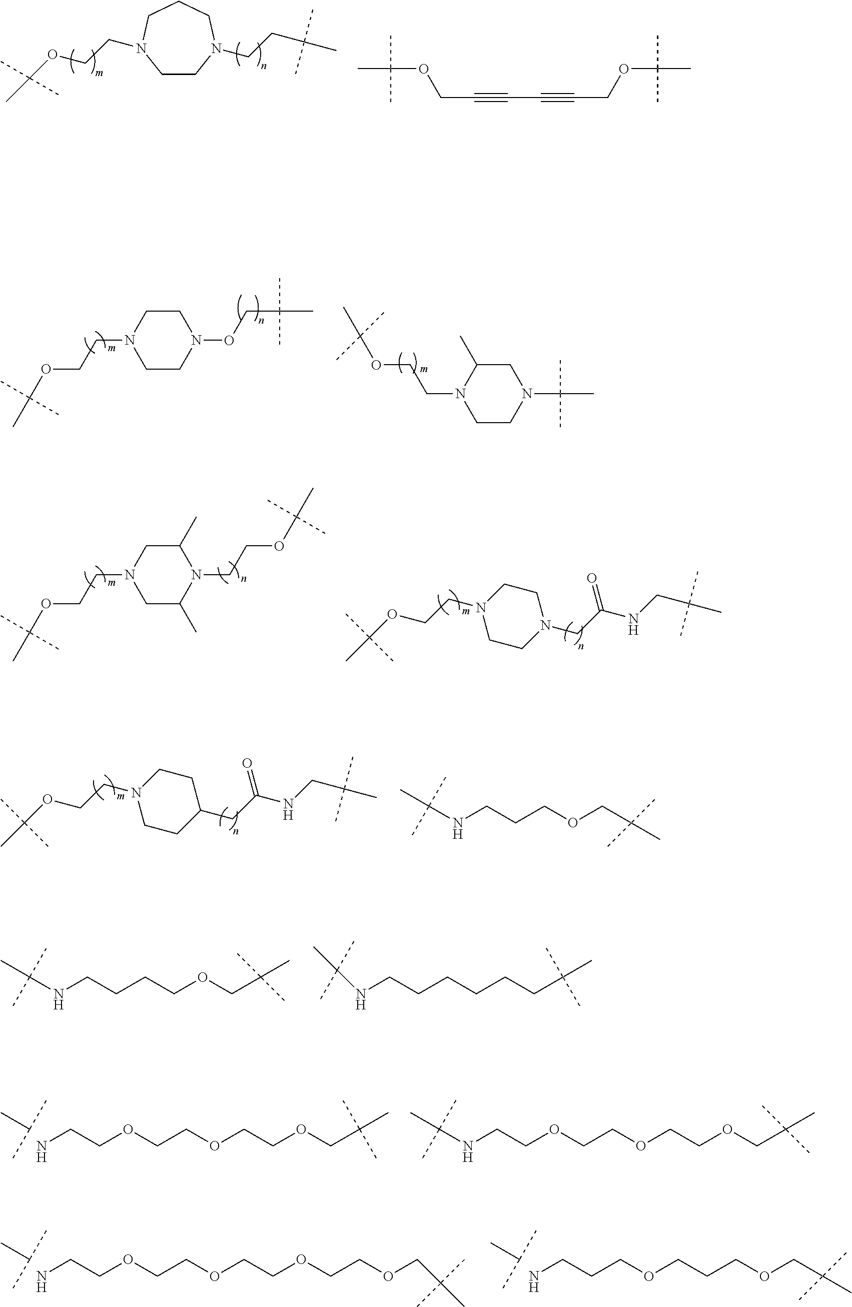

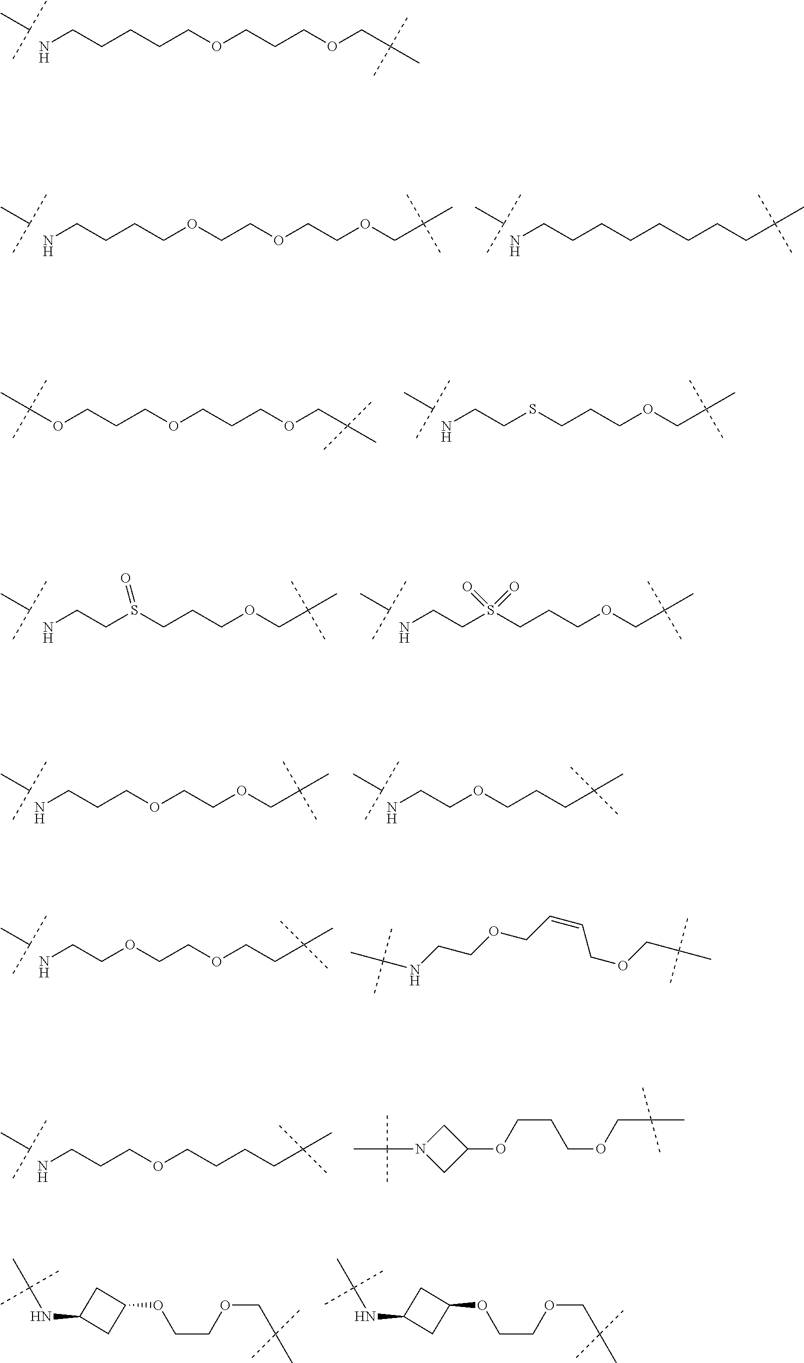

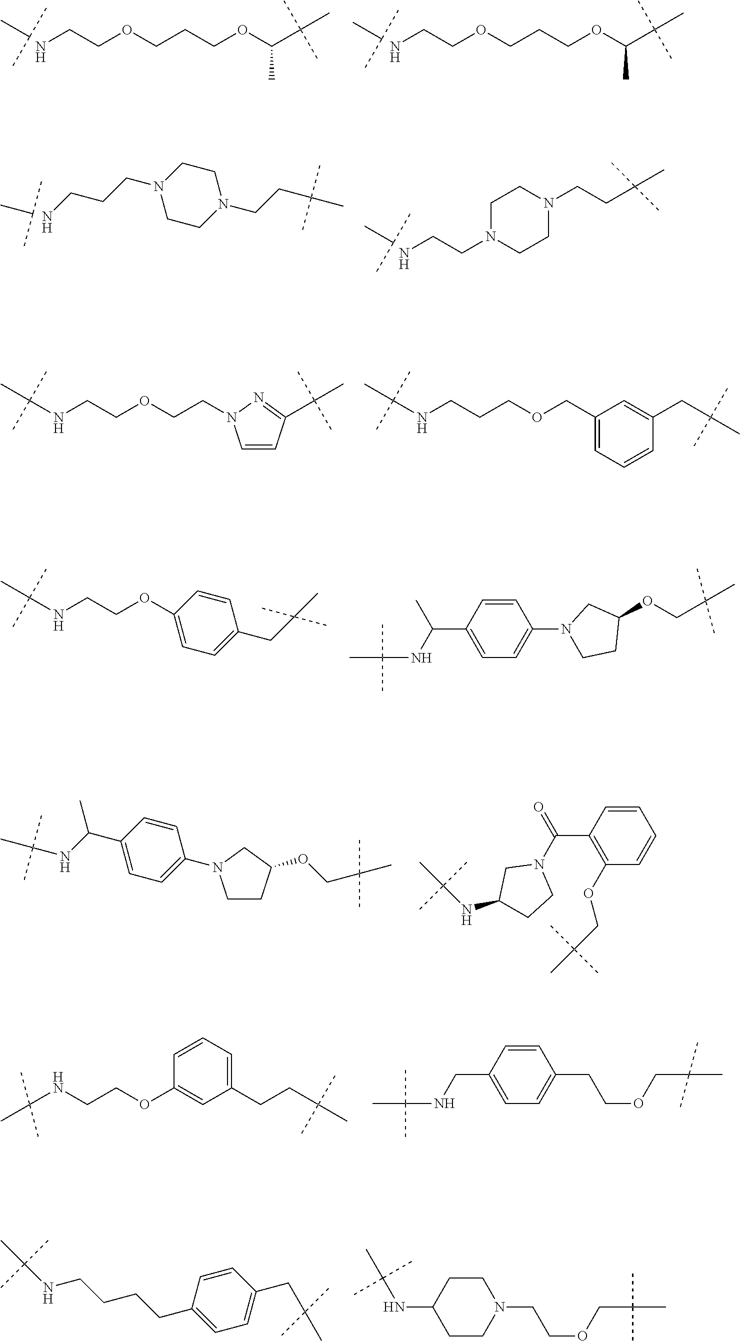

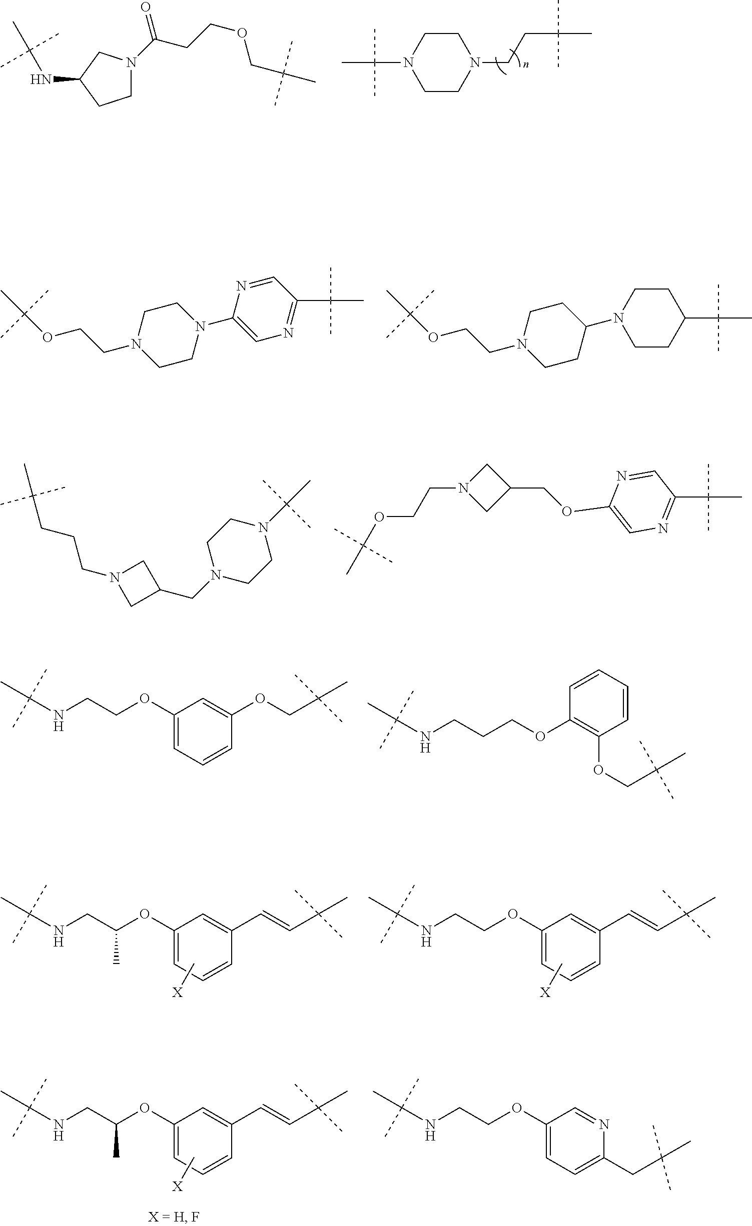

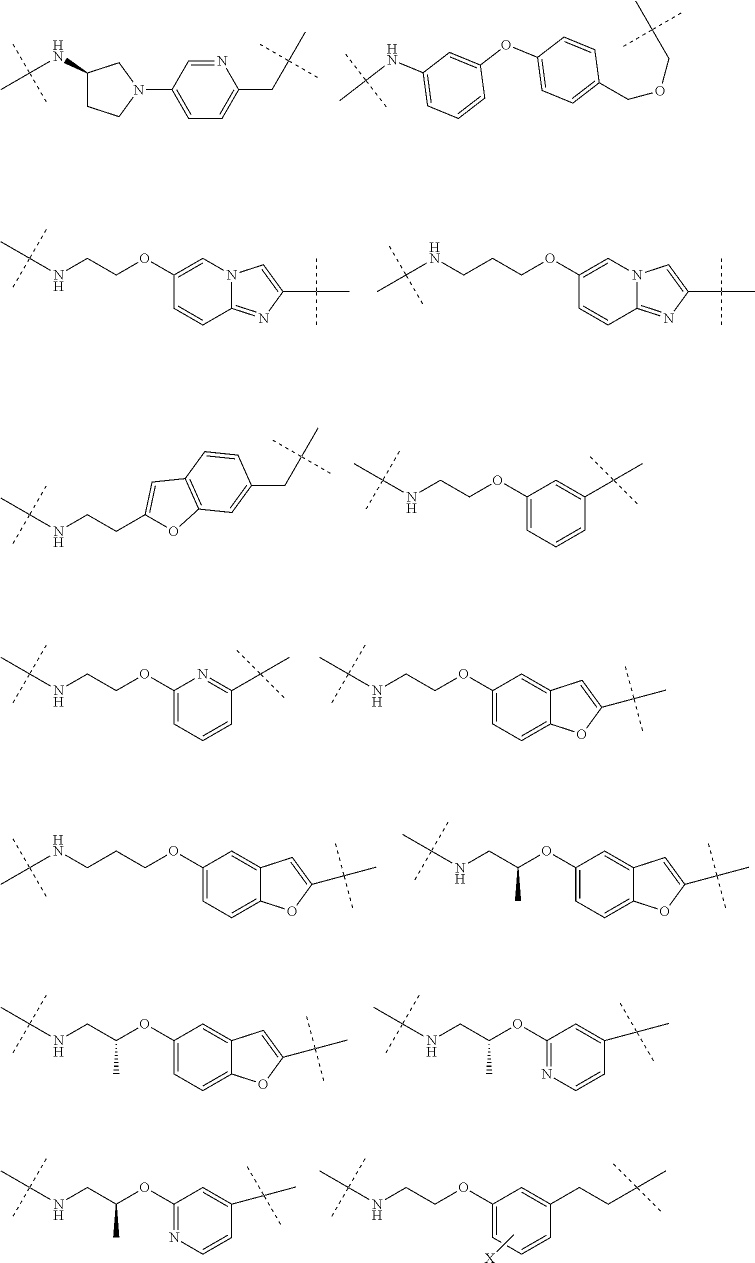

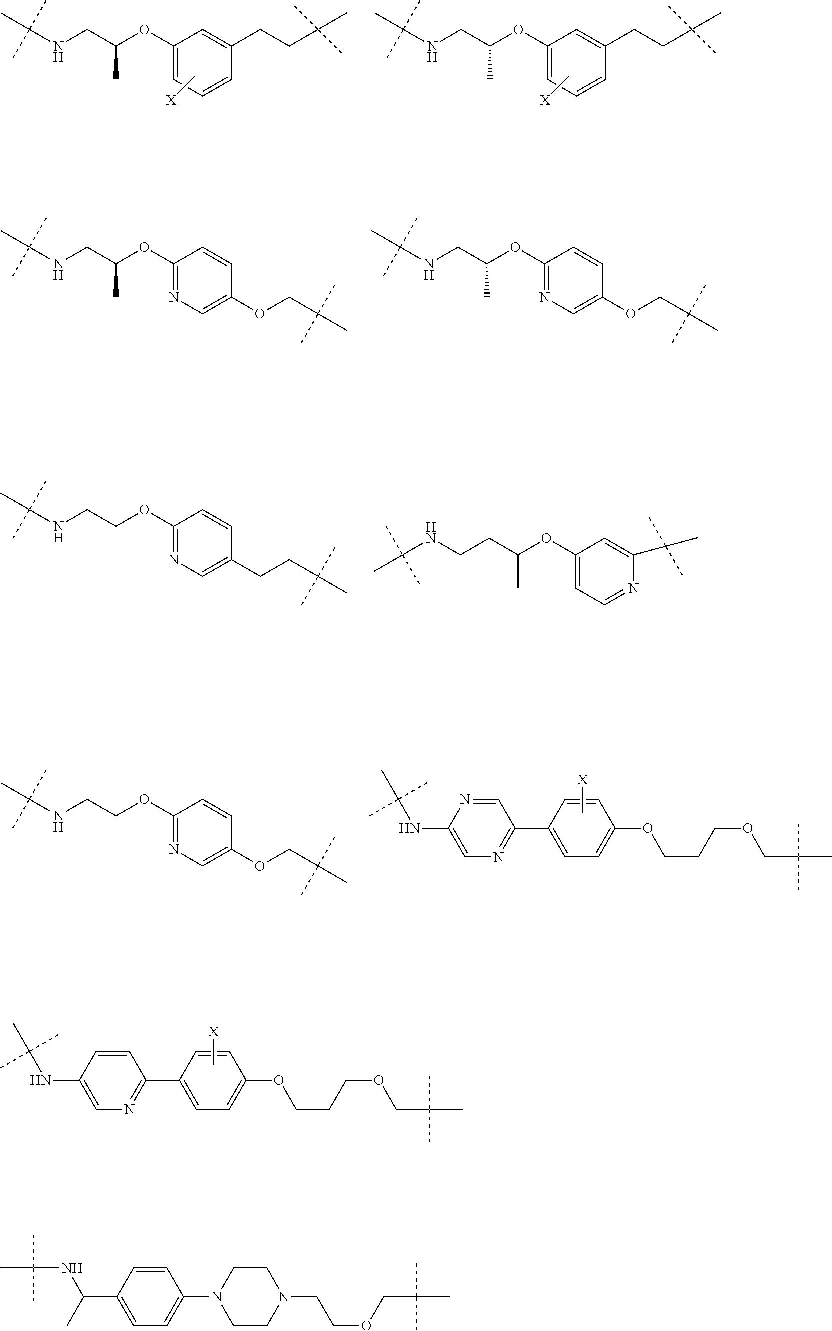

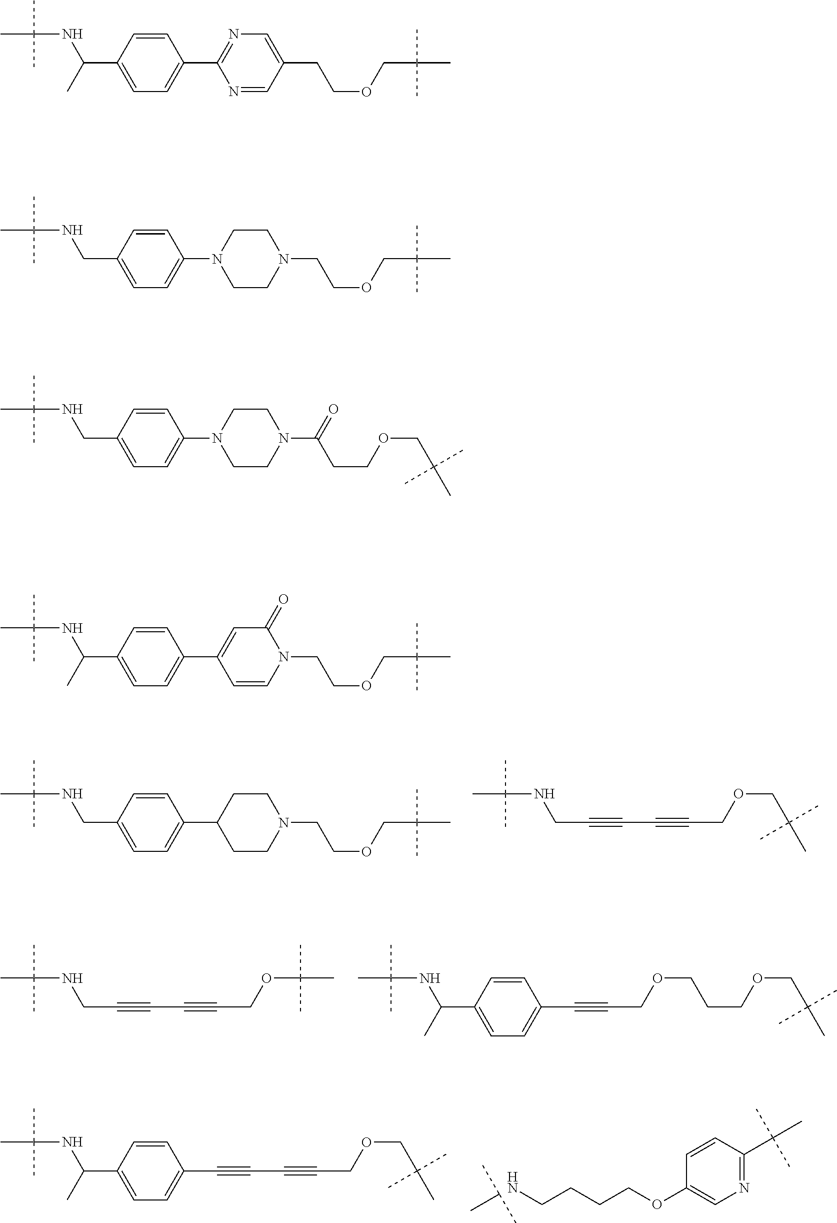

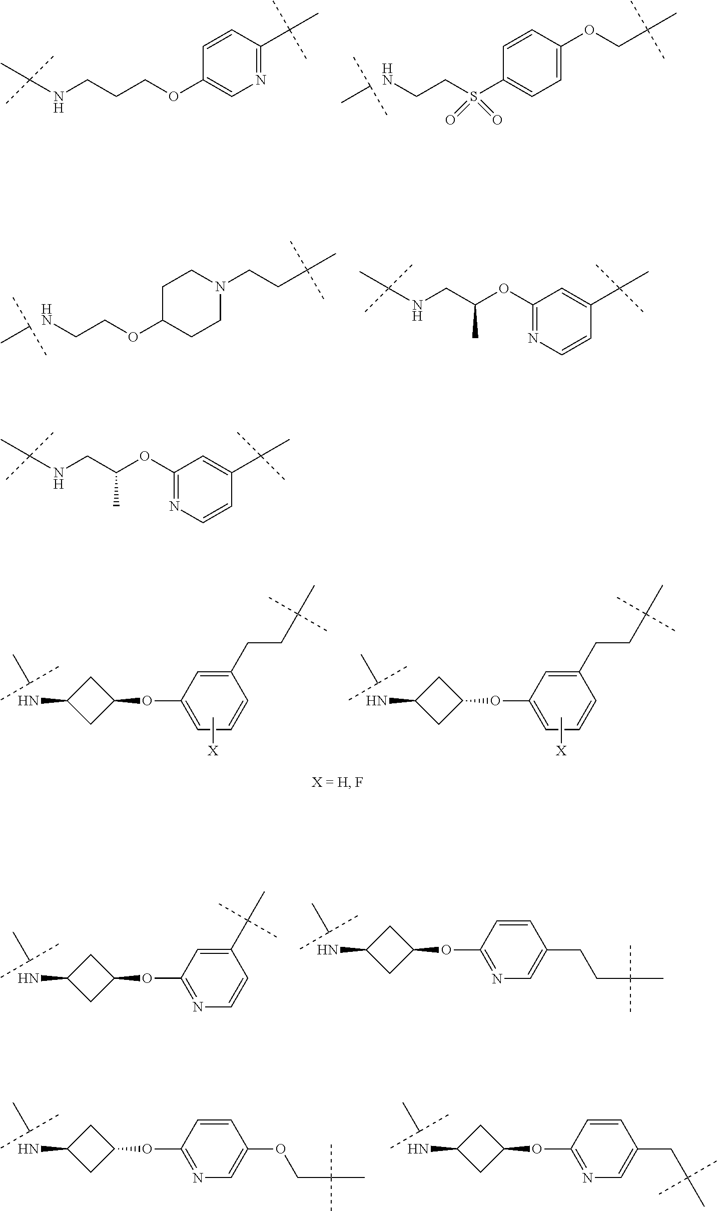

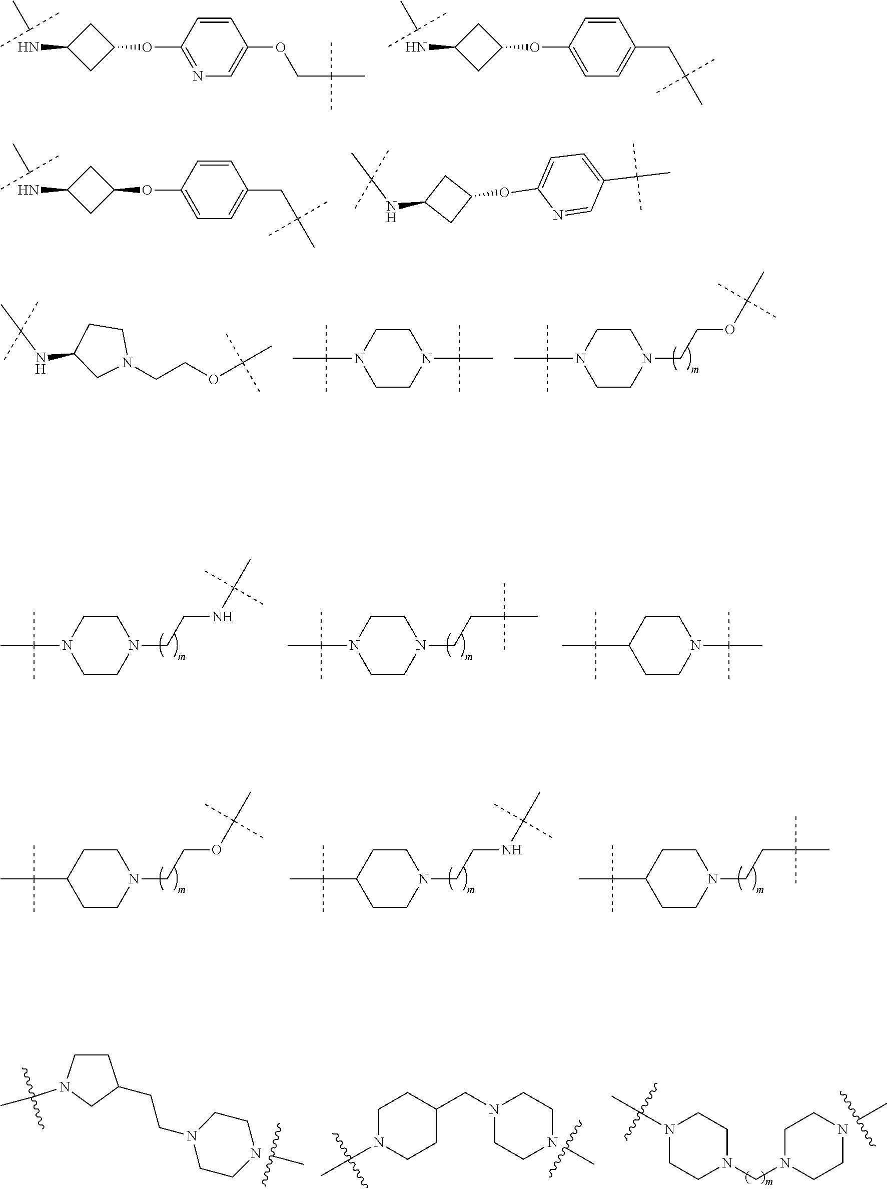

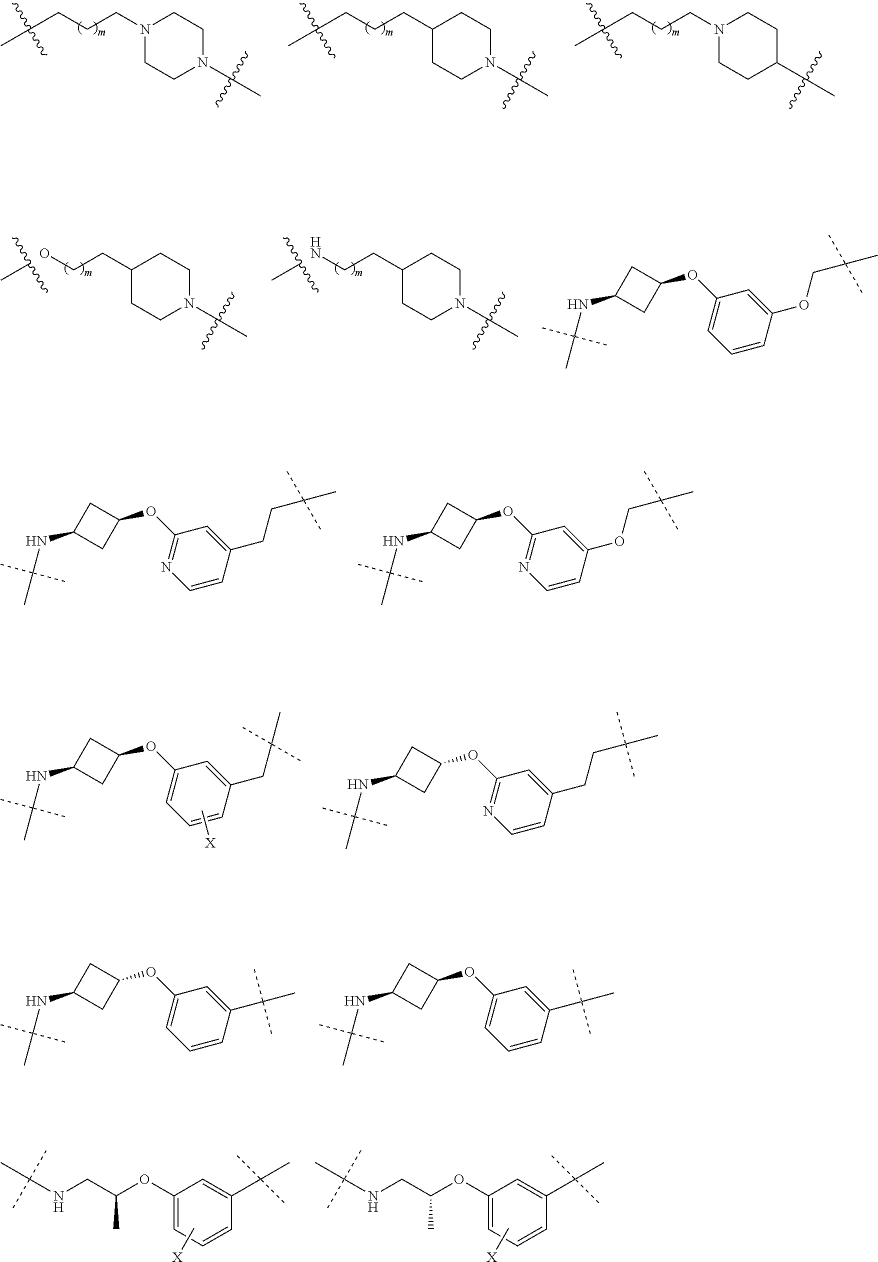

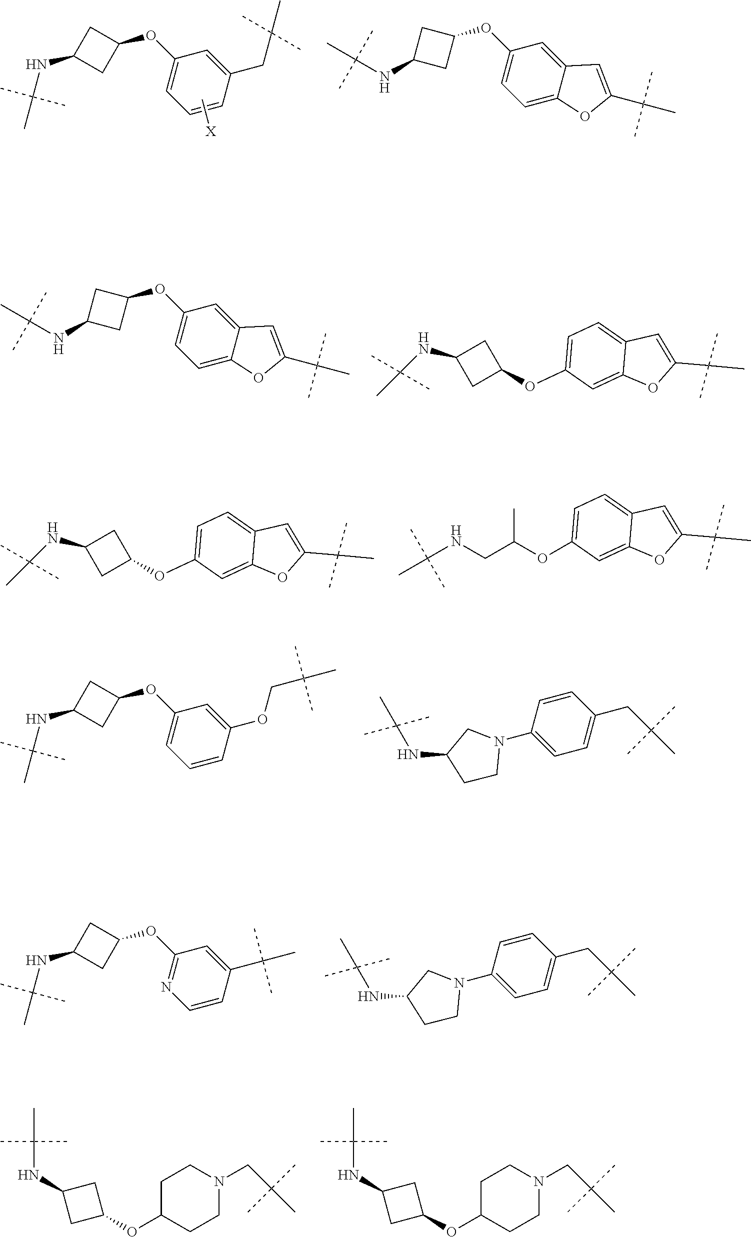

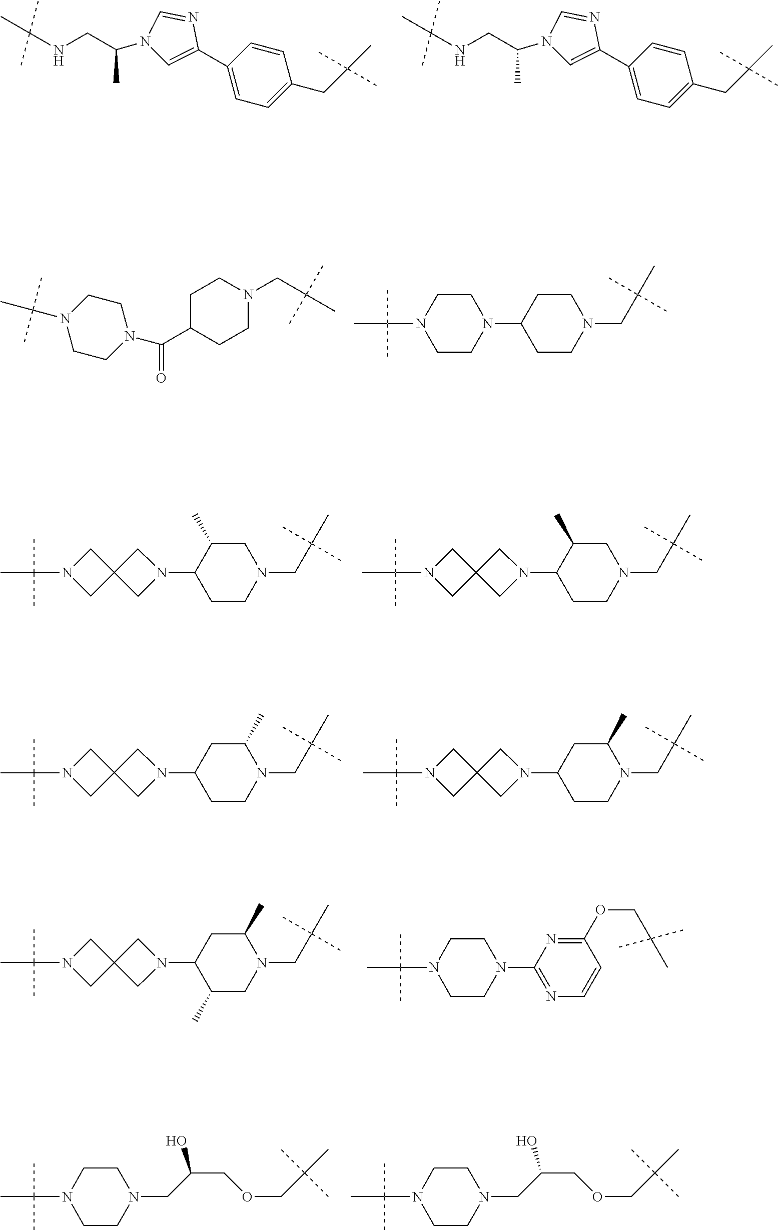

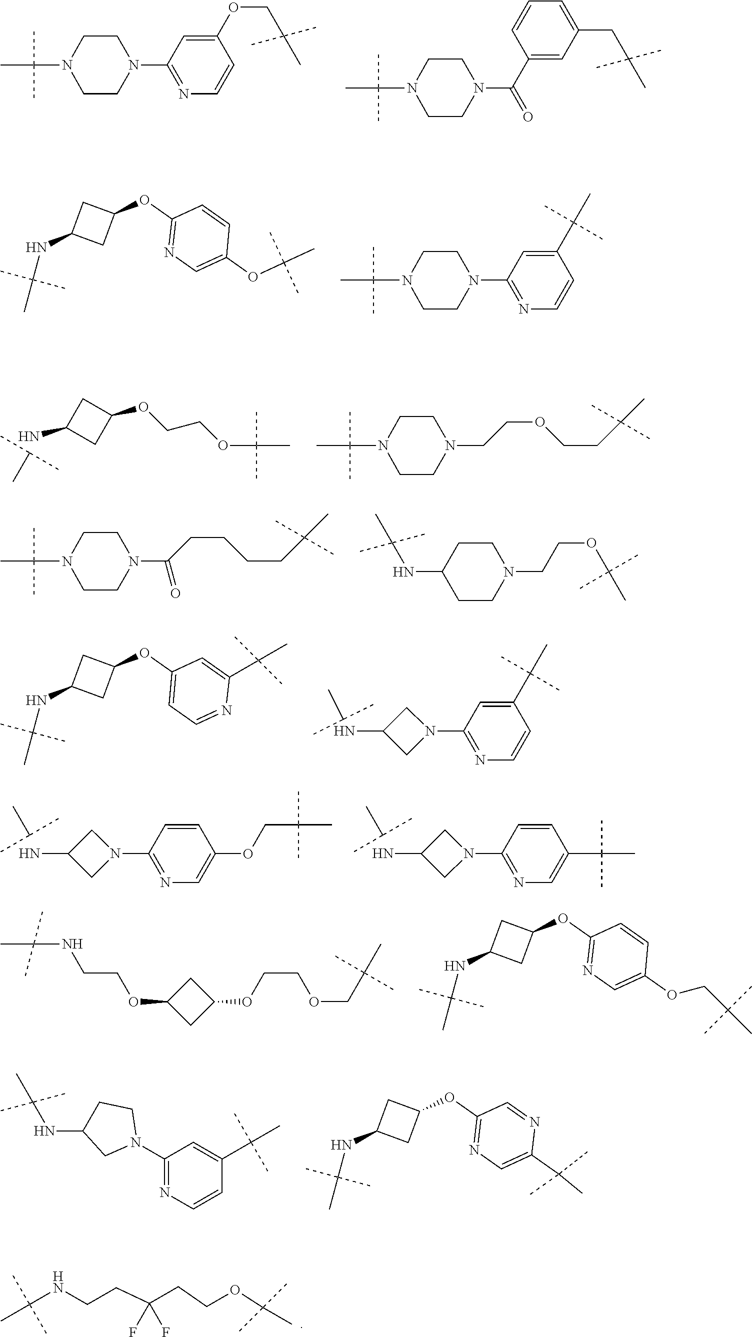

In any aspect or embodiment described herein, the linker (L) is selected from the group consisting of:

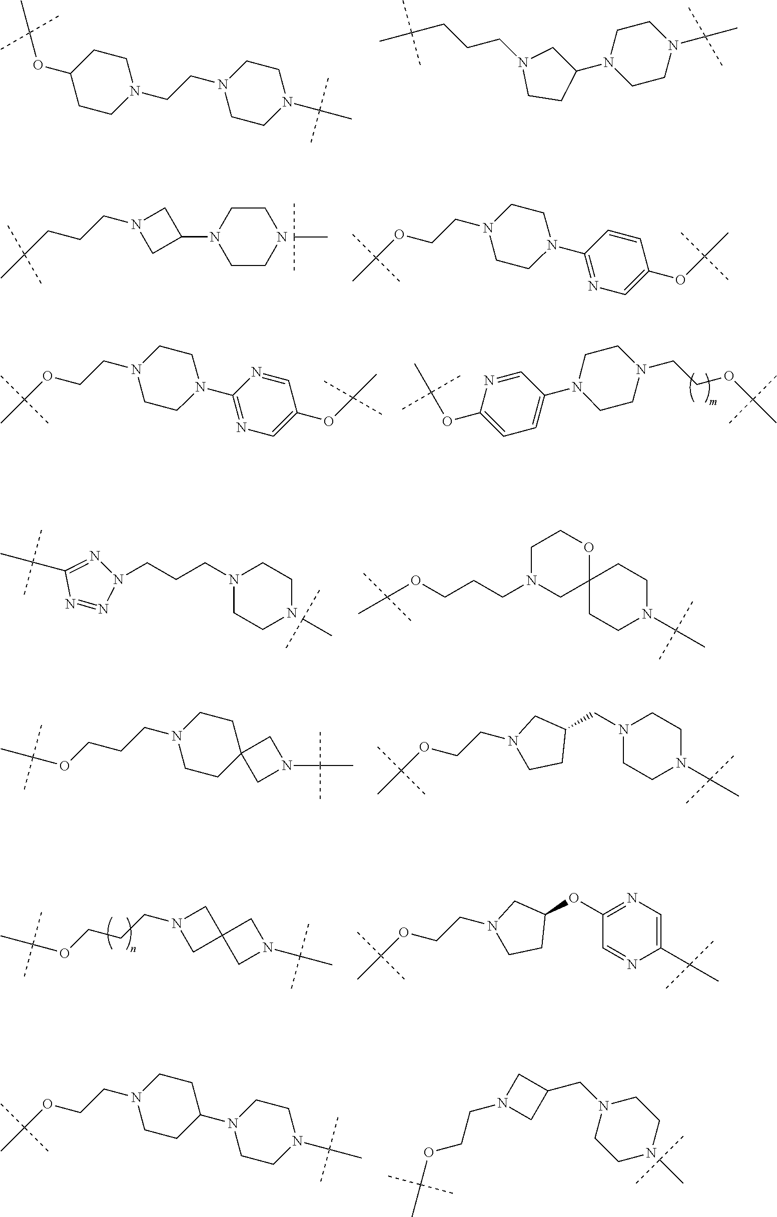

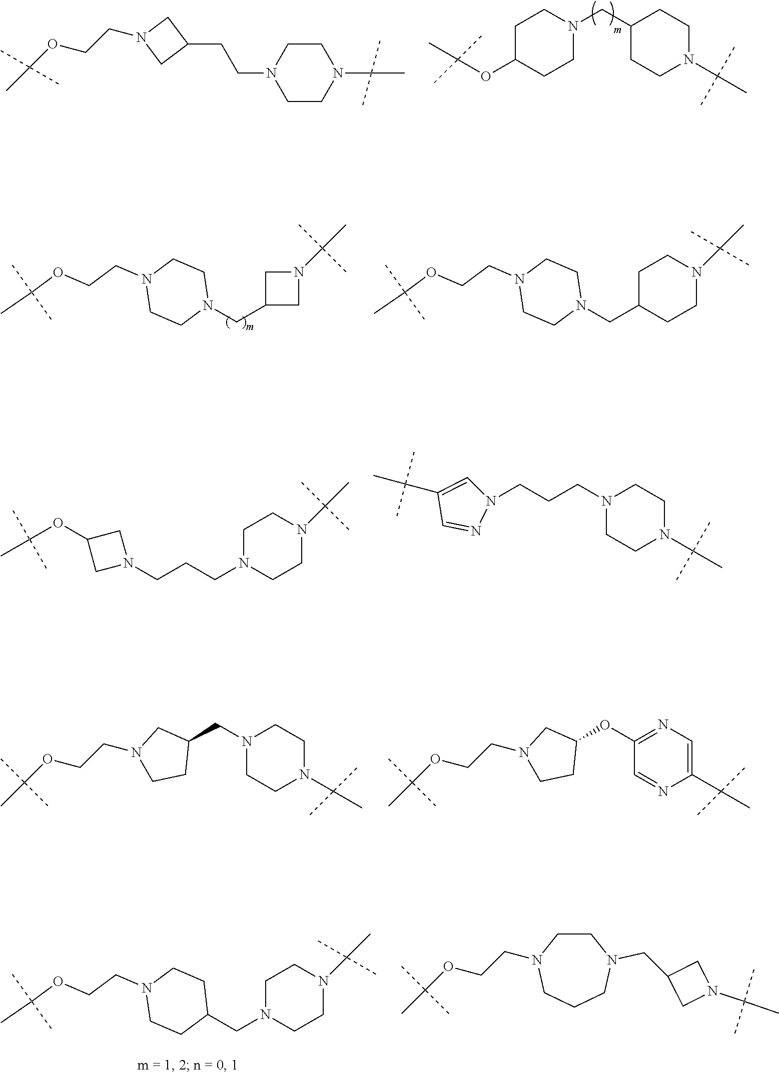

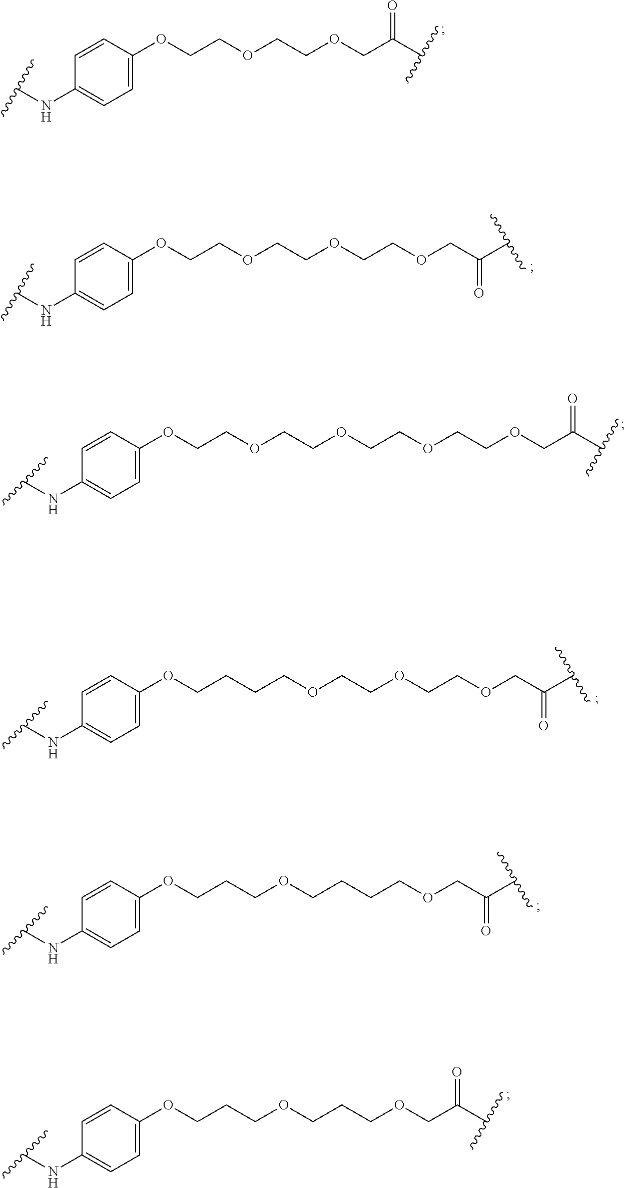 [SMILES: *Nc1ccc(OCCOCCOCC(*)=O)cc1 ; *Nc1ccc(OCCOCCOCCOCC(*)=O)cc1 ; *Nc1ccc(OCCOCCOCCOCCOCC(*)=O)cc1 ; *Nc1ccc(OCCCCOCCOCCOCC(*)=O)cc1 ; *Nc1ccc(OCCCOCCCCOCC(*)=O)cc1 ; *Nc1ccc(OCCCOCCCOCC(*)=O)cc1]

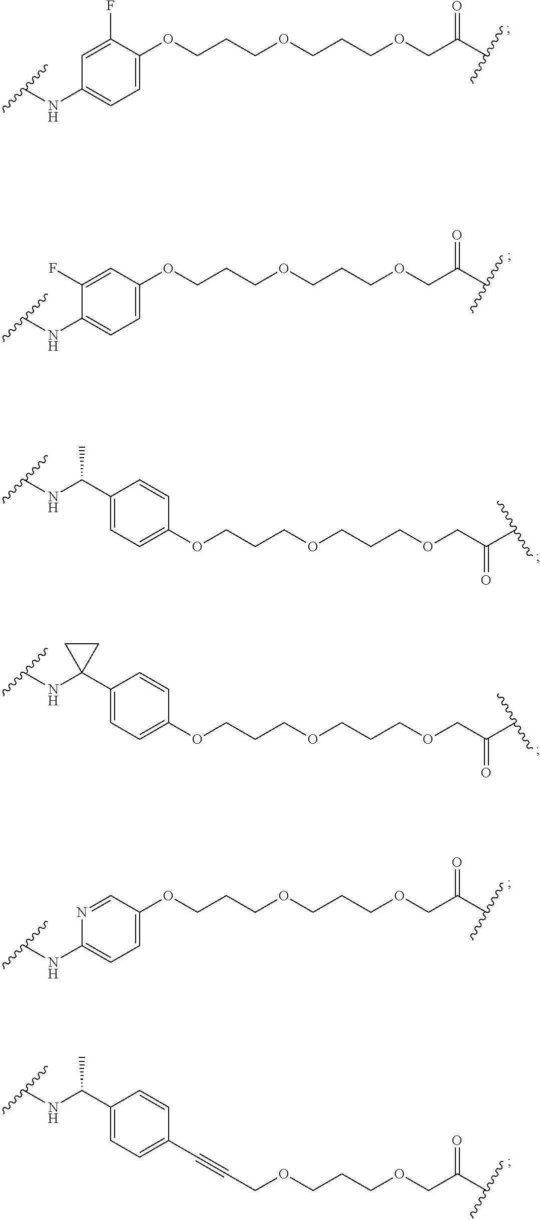 [SMILES: *Nc1ccc(OCCCOCCCOCC(*)=O)c(F)c1 ; *Nc1ccc(OCCCOCCCOCC(*)=O)cc1F ; *N[C@H](C)c1ccc(OCCCOCCCOCC(*)=O)cc1 ; *NC1(c2ccc(OCCCOCCCOCC(*)=O)cc2)CC1 ; *Nc1ccc(OCCCOCCCOCC(*)=O)cn1 ; *N[C@H](C)c1ccc(C#CCOCCCOCC(*)=O)cc1]

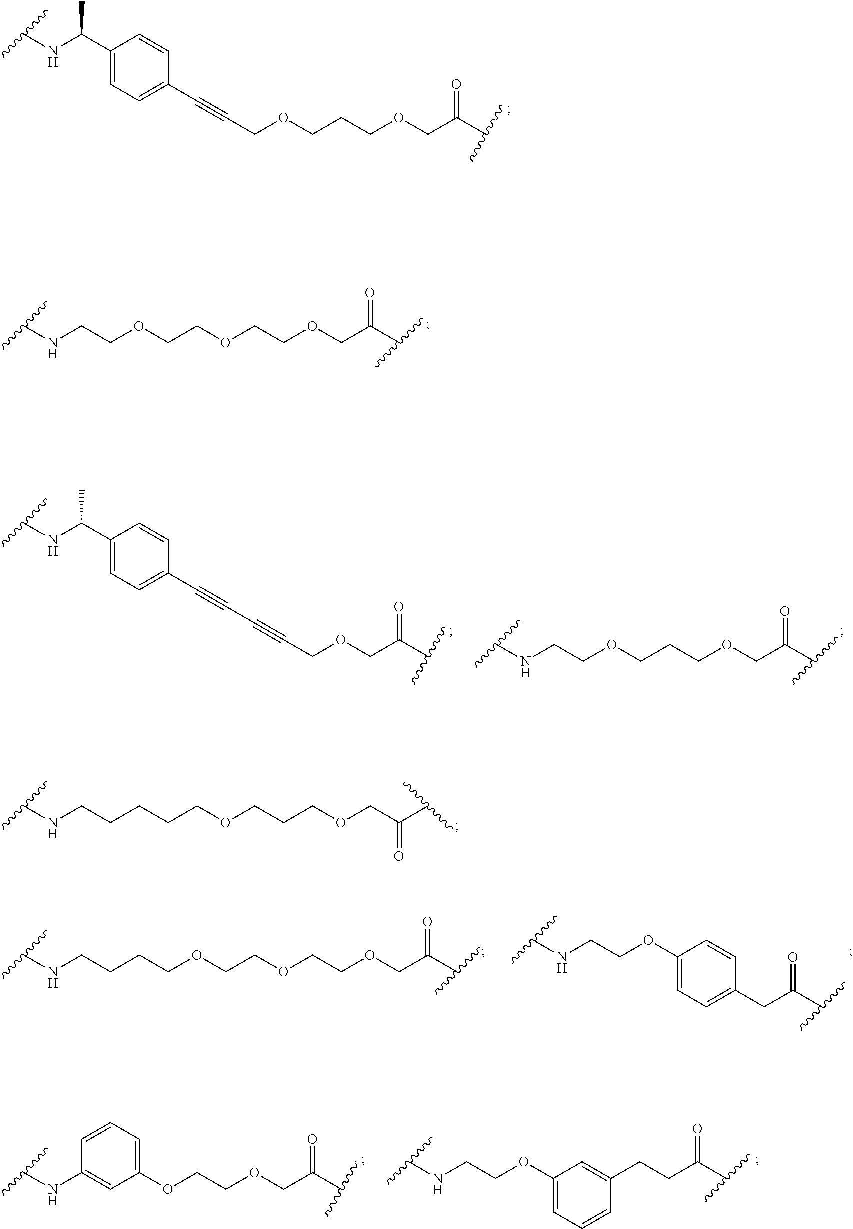

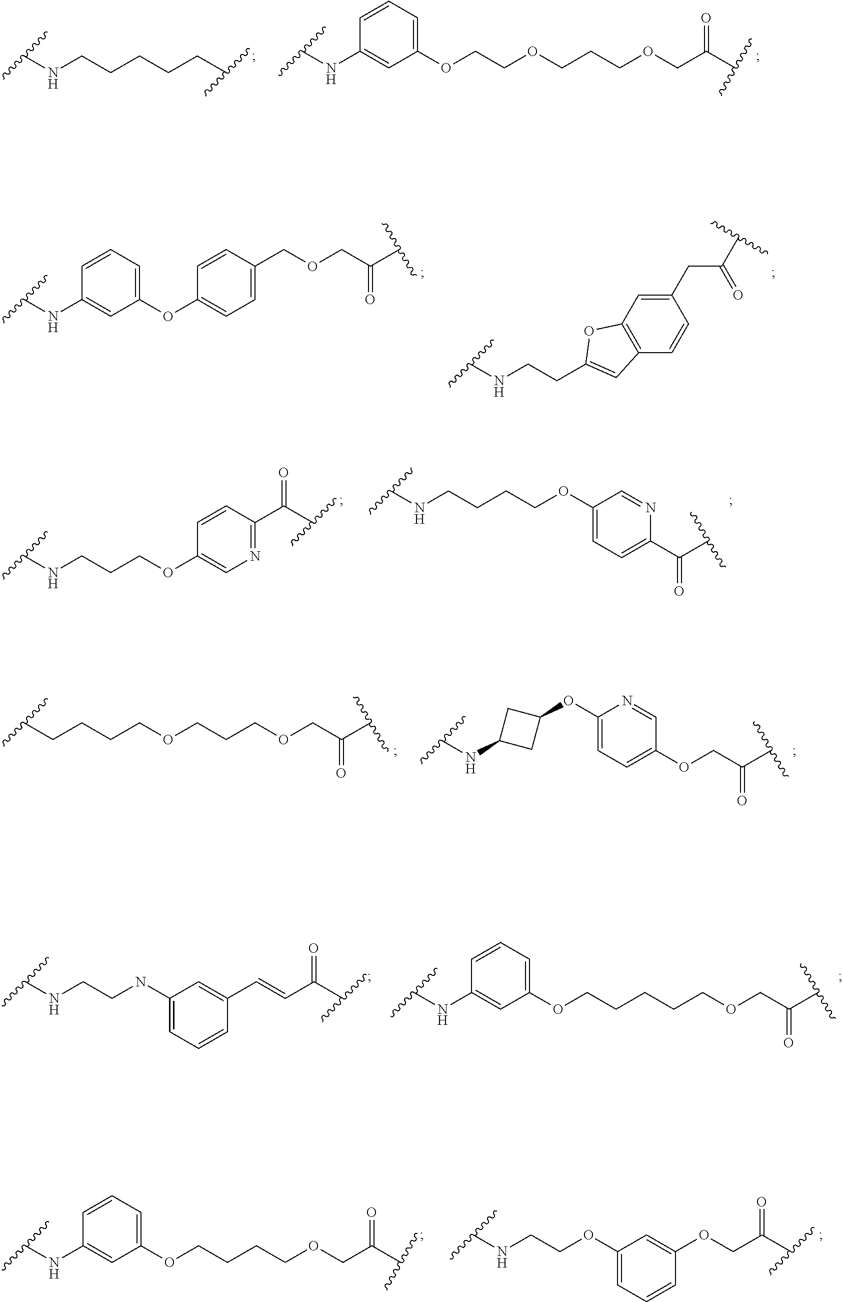

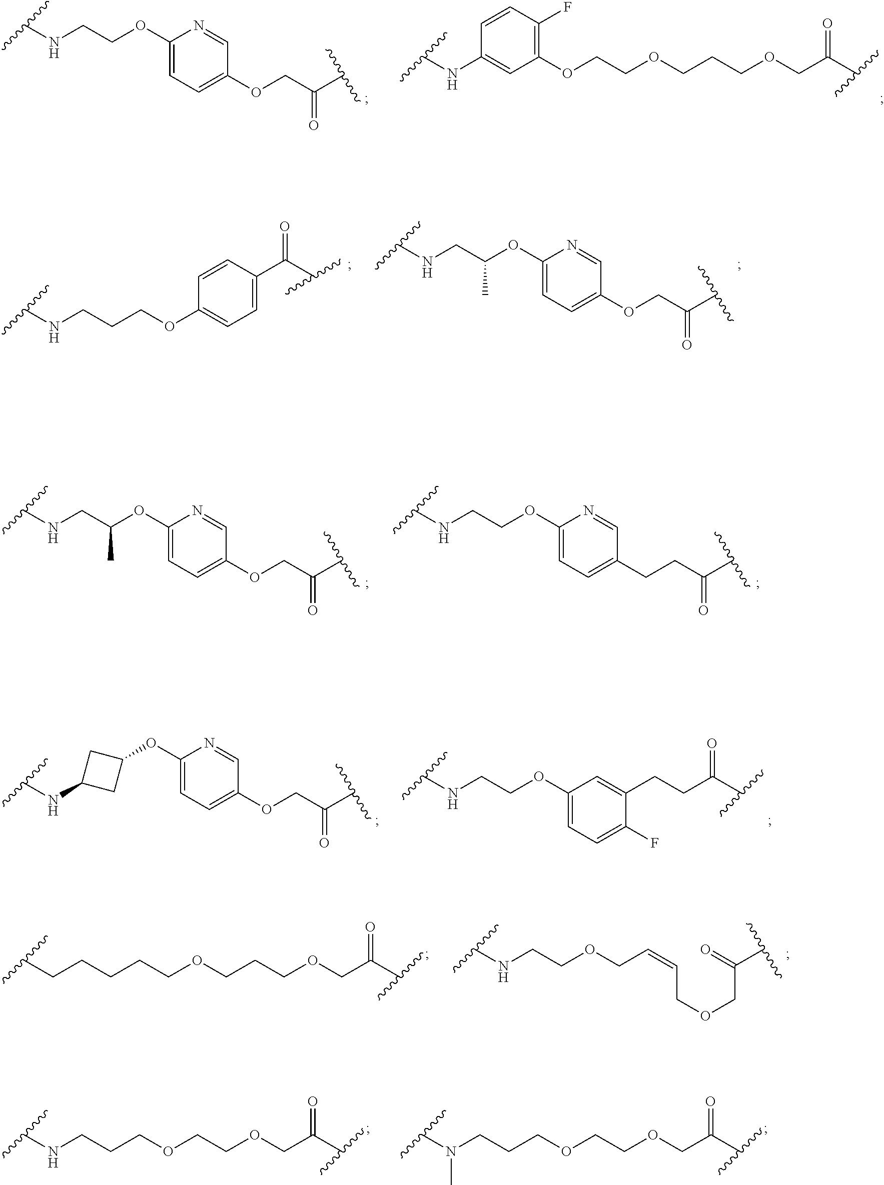

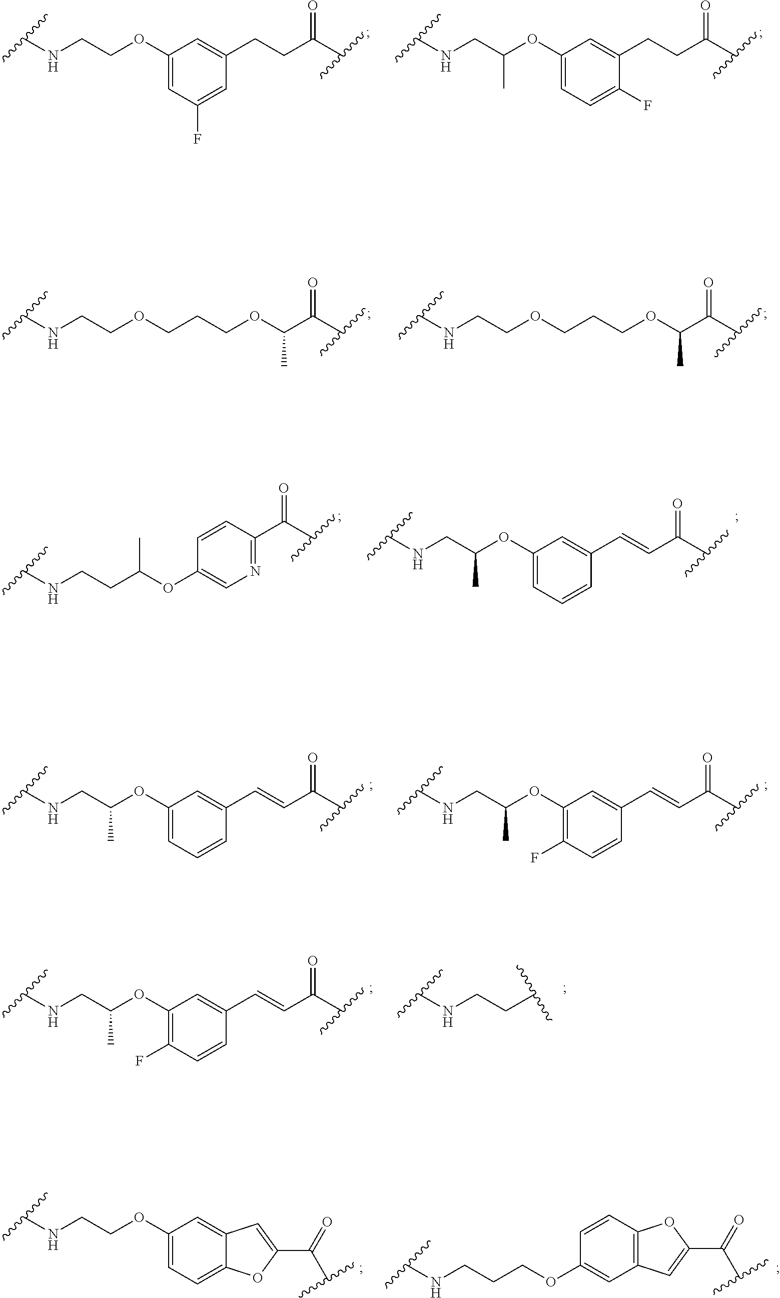

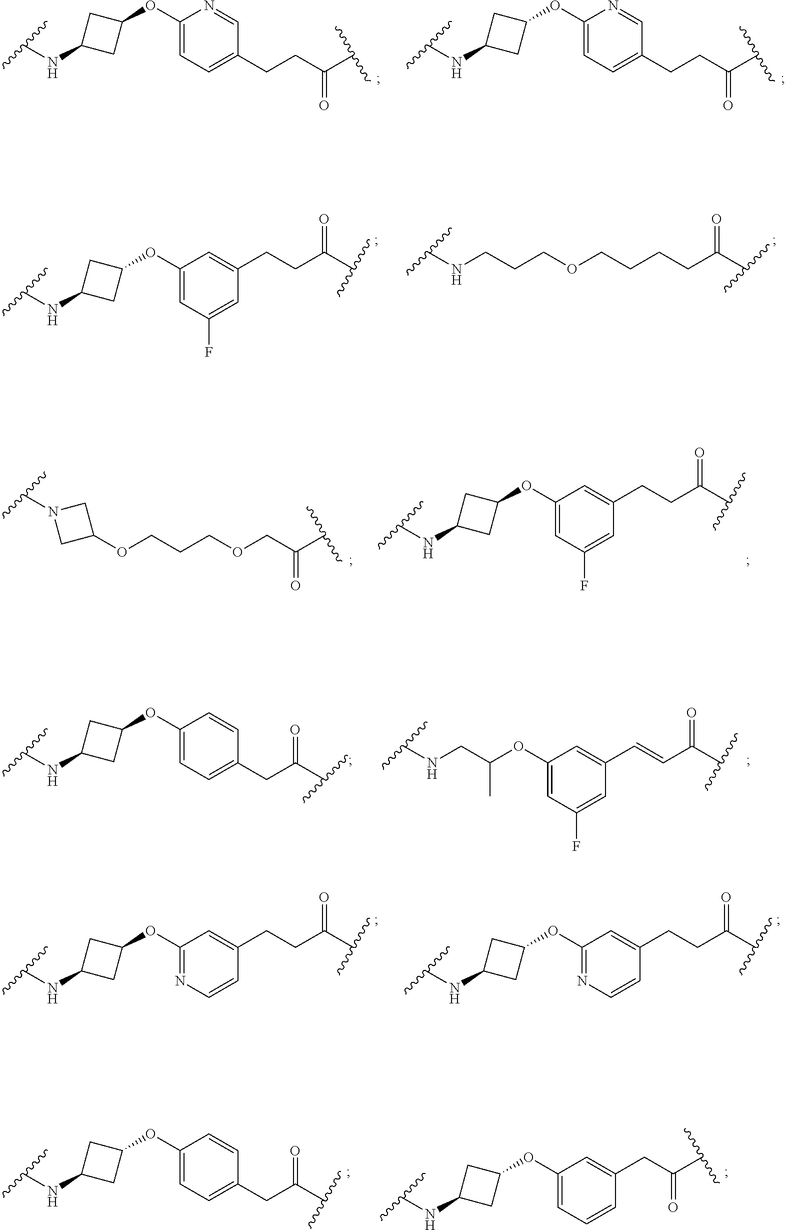

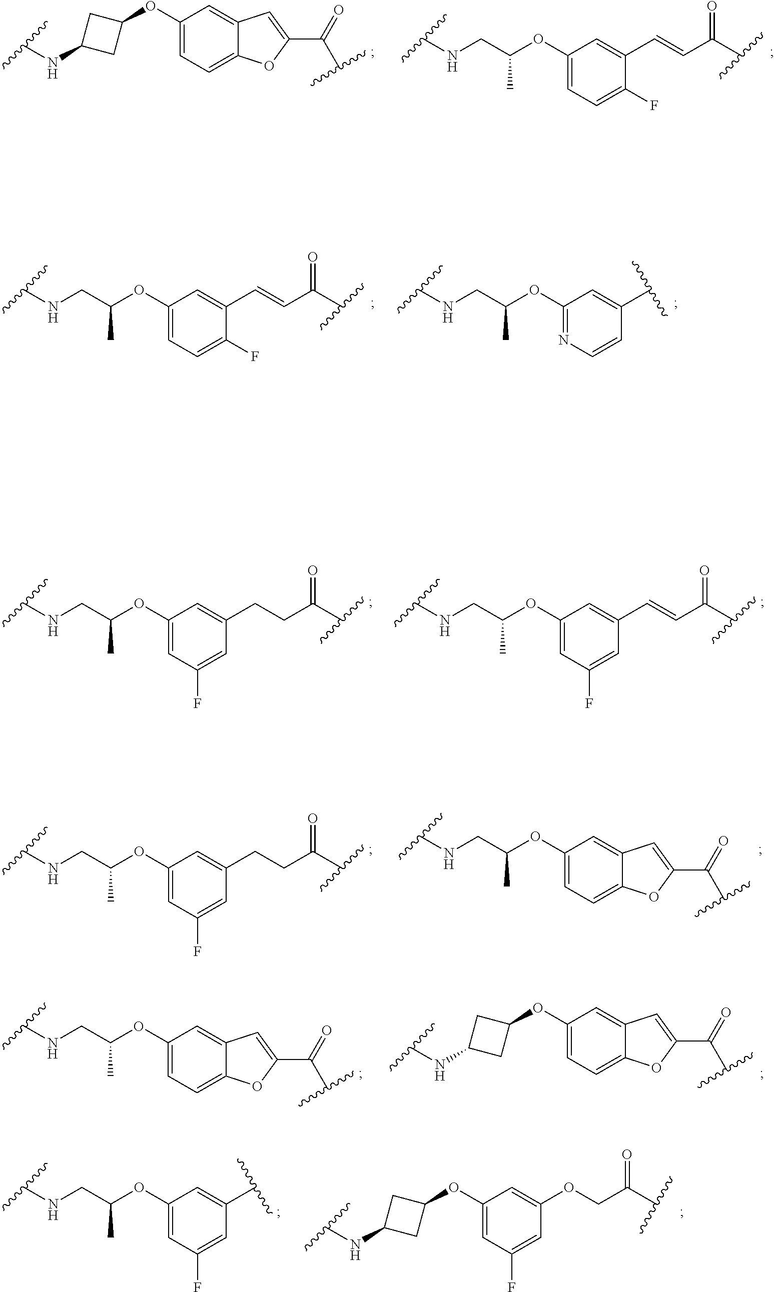

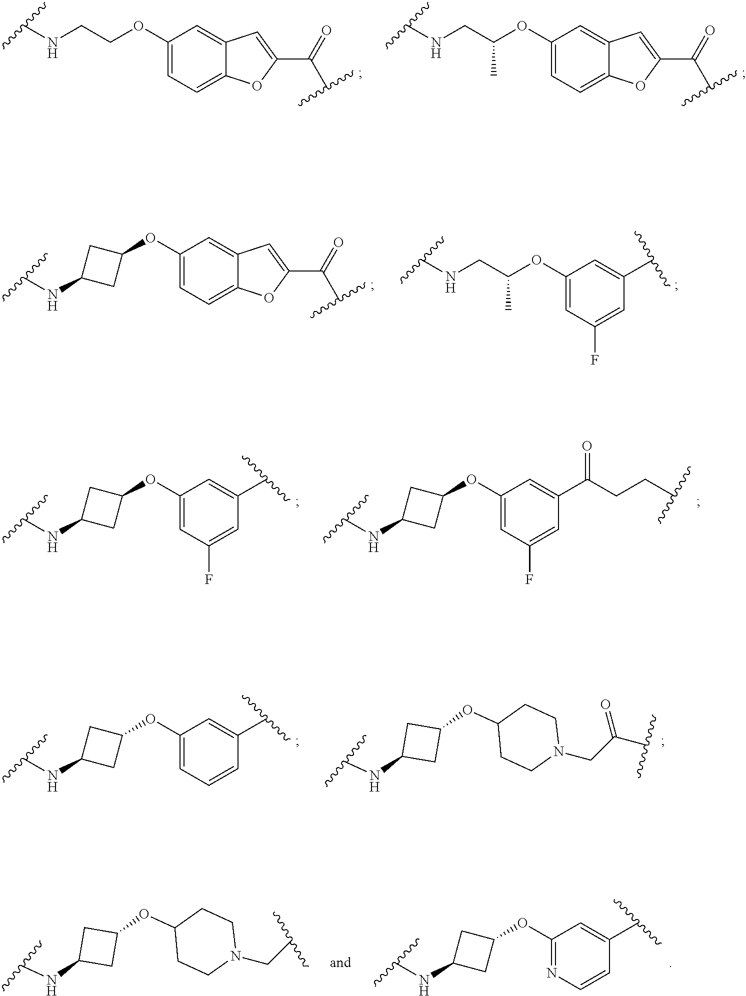

In any aspect or embodiment described herein, the linker (L) is selected from the group consisting of:

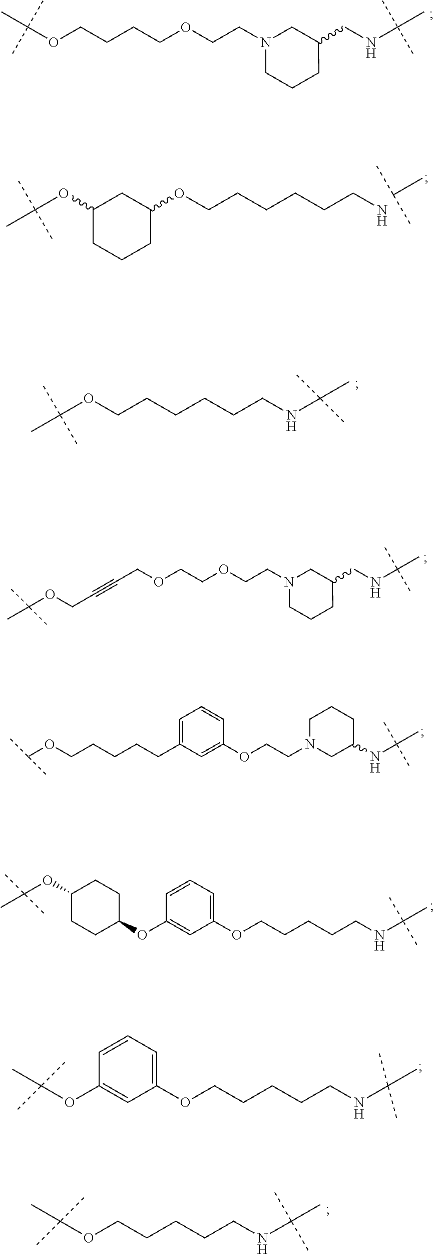

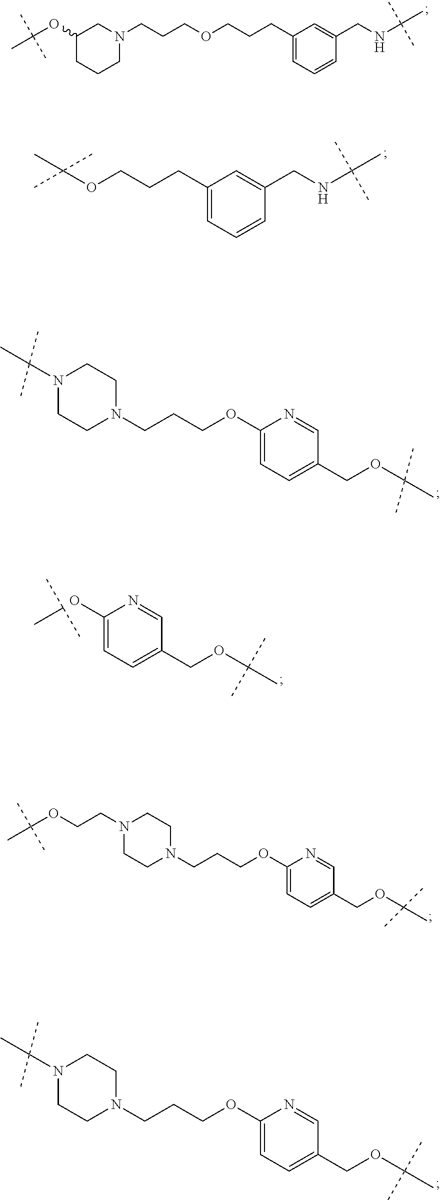

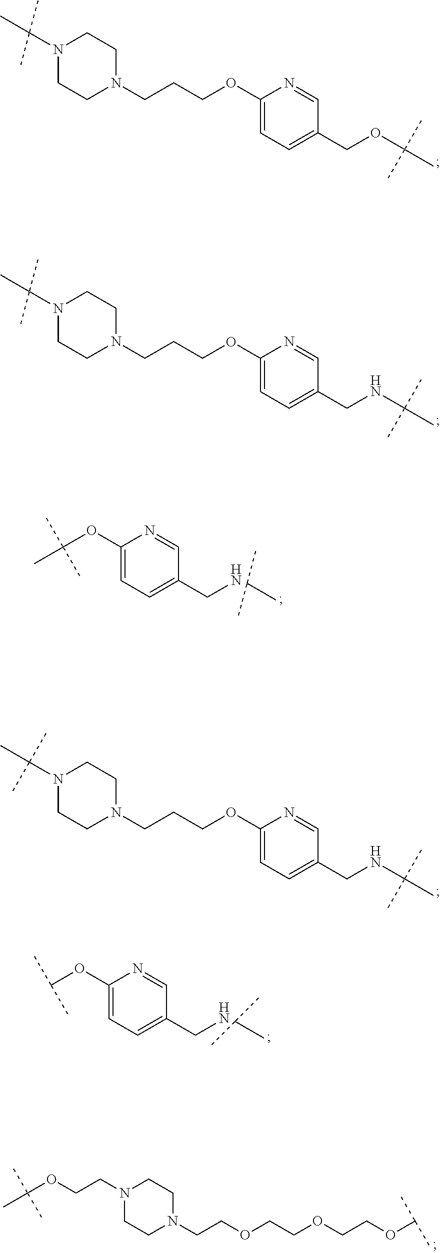

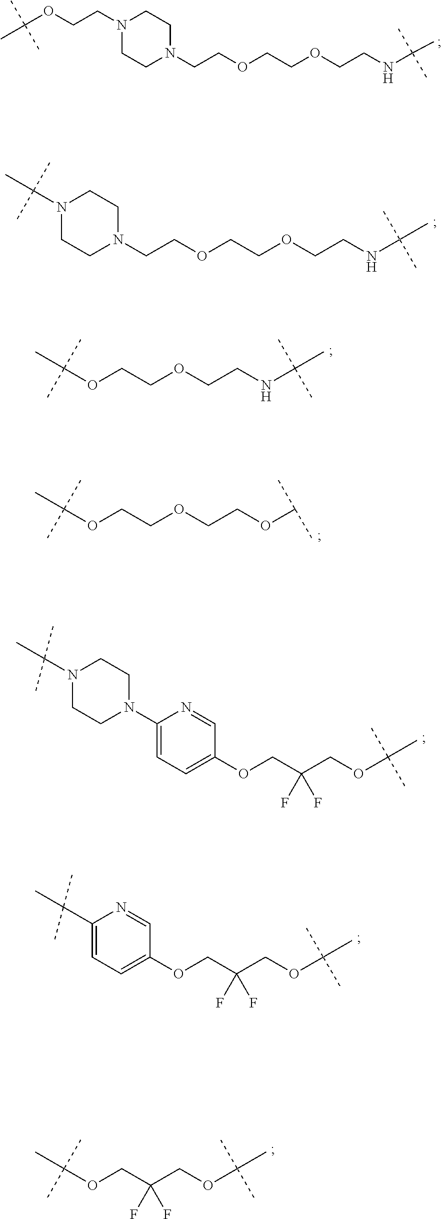

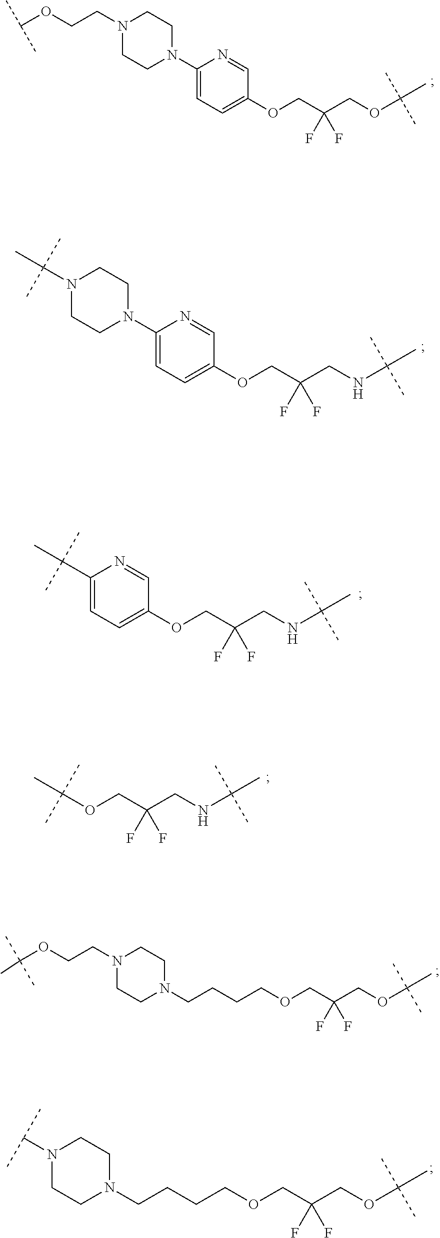

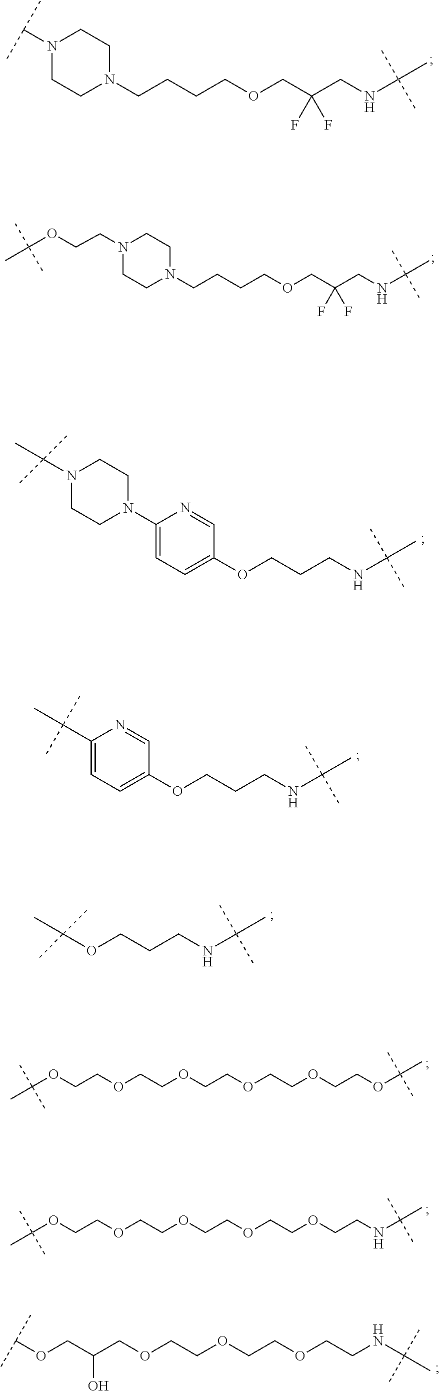

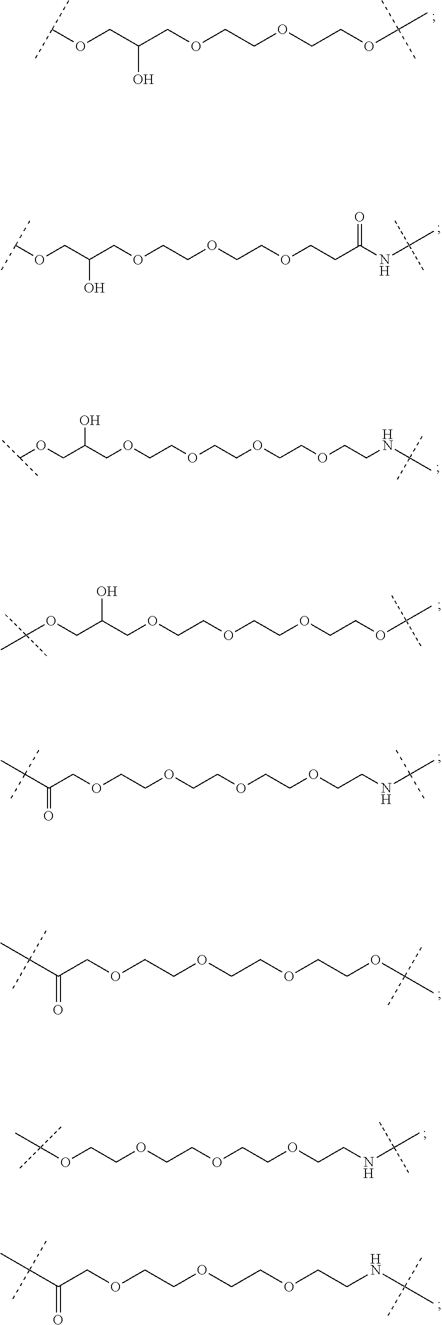

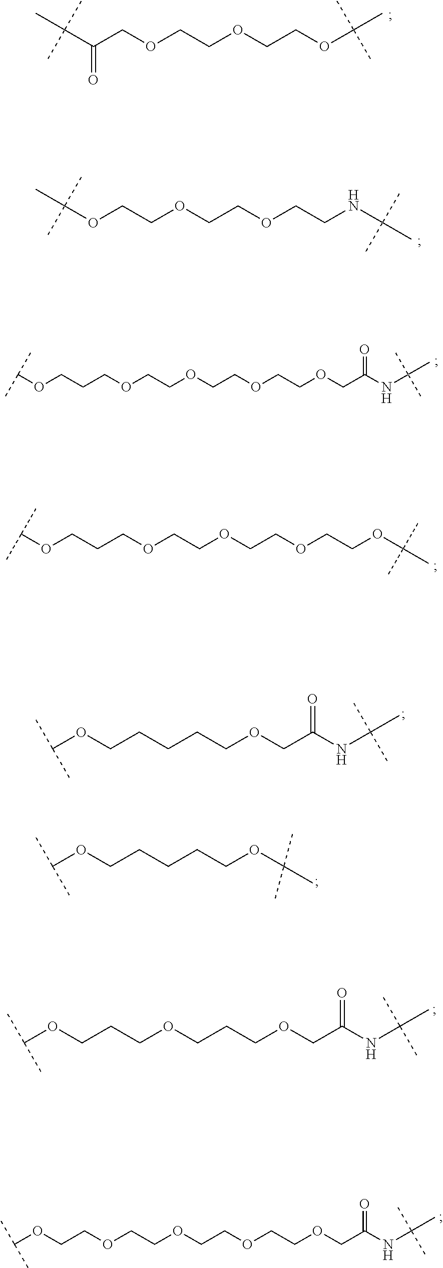

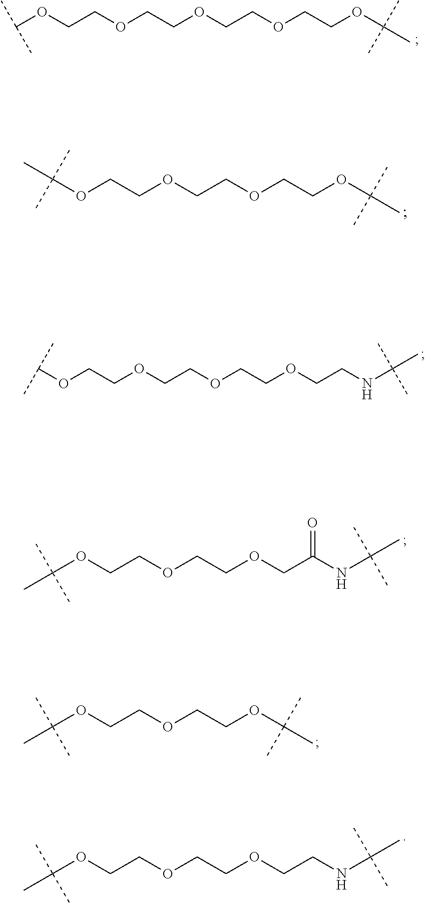

In any aspect or embodiment described herein, the linker (L) comprises a structure selected from, but not limited to the structure shown below, where a dashed line indicates the attachment point to the PTM or ULM moieties:

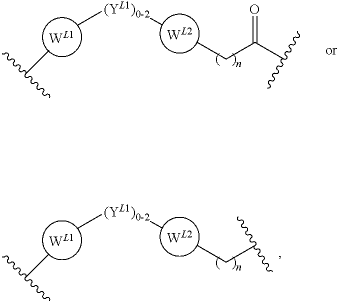

wherein:
W^{L1} and W^{L2} are each independently absent, a 4-8 membered ring with 0-4 heteroatoms, optionally substituted with R^{Q}, each R^{Q} is independently a H, halo, OH, CN, CF₃, C₁-C₆ alkyl (linear, branched, optionally substituted), C₁-C₆ alkoxy (linear, branched, optionally substituted), or 2 R^{Q} groups taken together with the atom they are attached to, form a 4-8 membered ring system containing 0-4 heteroatoms;
Y^{L1} is each independently a bond, C₁-C₆ alkyl (linear, branched, optionally substituted) and optionally one or more C atoms are replaced with O; or C₁-C₆ alkoxy (linear, branched, optionally substituted);
n is 0-10; and
a dashed line indicates the attachment point to the PTM or ULM moieties.

In any aspect or embodiment described herein, the linker (L) comprises a structure selected from, but not limited to the structure shown below, where a dashed line indicates the attachment point to the PTM or ULM moieties:

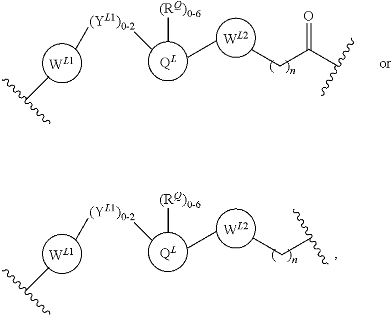

wherein:
W^{L1} and W^{L2} are each independently absent, aryl, heteroaryl, cyclic, heterocyclic, C₁₋₆ alkyl and optionally one or more C atoms are replaced with O, C₁₋₆ alkene and optionally one or more C atoms are replaced with O, C₁₋₆ alkyne and optionally one or more C atoms are replaced with O, bicyclic, biaryl, biheteroaryl, or biheterocyclic, each optionally substituted with R^{Q}, each R^{Q} is independently a H, halo, OH, CN, CF₃, hydroxyl, nitro, C ≡CH, C₂₋₆ alkenyl, C₂₋₆ alkynyl, C₁-C₆ alkyl (linear, branched, optionally substituted), C₁-C₆ alkoxy (linear, branched, optionally substituted), OC₁₋₃alkyl (optionally substituted by 1 or more —F), OH, NH₂, NR^{Y1}R^{Y2}, CN, or 2 R^{Q} groups taken together with the atom they are attached to, form a 4-8 membered ring system containing 0-4 heteroatoms;
Y^{L1} is each independently a bond, NR^{YL1}, O, S, NR^{YL2}, CR^{YL1}R^{YL2}, C═O, C═S, SO, SO₂, C₁-C₆ alkyl (linear, branched, optionally substituted) and optionally one or more C atoms are replaced with O; C₁-C₆ alkoxy (linear, branched, optionally substituted);
Q^{L} is a 3-6 membered alicyclic or aromatic ring with 0-4 heteroatoms, optionally bridged, optionally substituted with 0-6 R^{Q}, each R^{Q} is independently H, C₁₋₆ alkyl (linear, branched, optionally substituted by 1 or more halo, C₁₋₆ alkoxyl), or 2 R^{Q} groups taken together with the atom they are attached to, form a 3-8 membered ring system containing 0-2 heteroatoms);
R^{YL1}, R^{Y12} are each independently H, OH, C₁₋₆ alkyl (linear, branched, optionally substituted by 1 or more halo, C₁₋₆ alkoxyl), or R¹, R² together with the atom they are attached to, form a 3-8 membered ring system containing 0-2 heteroatoms);
n is 0-10; and
a dashed line indicates the attachment point to the PTM or ULM moieties.

In any aspect or embodiment described herein, the L is selected from:

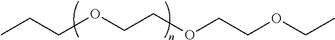

wherein n is an integer from 0 to 10;

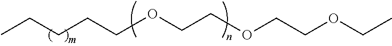

wherein n is an integer from 0 to 10, and m is an integer from 2 to 10; and

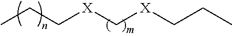

wherein n is an integer from 0 to 10, m is an integer from 0 to 10, and X is independently O or CH₂.

In any aspect or embodiment described herein, the L is a polyethylenoxy group optionally substituted with aryl or phenyl comprising from 1 to 10 ethylene glycol units.

In any aspect or embodiment described herein, the compound comprises multiple ULMs, multiple PTMs, multiple linkers or any combinations thereof.

In any aspect or embodiment described herein, the compound is selected from the group consisting of exemplary compounds 1-50.

In another aspect, the present disclosure provides a composition comprising an effective amount of a bifunctional compound of the present disclosure and a pharmaceutically acceptable carrier.

In any aspect or embodiment described herein, the composition further comprises at least one of additional bioactive agent or another bifunctional compound of the present disclosure

In any aspect or embodiment described herein, the additional bioactive agent is anti-cancer agent.

According to a further aspect, the present disclosure provides a composition comprising a pharmaceutically acceptable carrier and an effective amount of at least one compound of the present disclosure for treating a disease or disorder in a subject, the method comprising administering the composition to a subject in need thereof, wherein the compound is effective in treating or ameliorating at least one symptom of the disease or disorder.

In any aspect or embodiment described herein, the disease or disorder is associated with c-Met accumulation and aggregation.

In any aspect or embodiment described herein, the disease or disorder is cancer associated with c-Met accumulation and aggregation.

In any aspect or embodiment described herein, the disease or disorder is at least one of: gastric cancer, non-small cell lung cancer, squamous-cell carcinoma, basal cell carcinoma, adenocarcinoma, hepatocellular carcinomas, advanced hepatocellular carcinoma, renal cell carcinomas, and papillary renal cell carcinoma; cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas; T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML; or combinations thereof.

In any aspect or embodiment described herein, the disease or disorder is at least one of astric cancer, non-small cell lung cancer, advanced hepatocellular carcinoma (HCC), papillary renal cell cancer (RCC), or a combination thereof.

According to an additional aspect, the present disclosure provides a compound represented by the chemical structure PTM-L-ULM

the PTM is represented by the chemical structure:

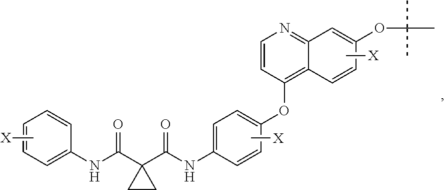

wherein each X is independently H, OMe, or F;

the linker (L) comprises a chemical structural unit represented by the formula:

-(A)_{q}-, wherein:
(A)_{q} is a group which is connected to a ULM or PTM moiety; and
   q is an integer greater than or equal to 1;
each A is independently selected from the group consisting of, a bond, CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₃₋₁₁heteocyclyl optionally substituted with 0-6 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R^{L5} groups; and
R^{L1}, R^{L2}, R^{L3}, R^{L4} and R^{L5} are, each independently, H, halo, C₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, S_{F5}, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NH SO₂NH(C₁₋₈alkyl), NH SO₂N(C₁₋₈alkyl)₂, NH SO₂NH₂;
   the ULM is represented by the chemical structure:

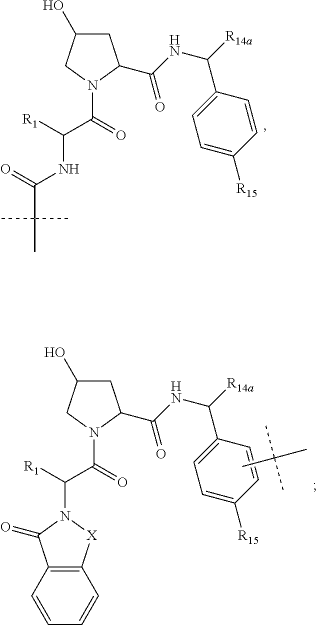

R₁ is H, ethyl, isopropyl, tert-butyl, sec-butyl, cyclopropyl, cyclobutyl, cyclopentyl, or cyclohexyl; optionally substituted alkyl, optionally substituted hydroxyalkyl, optionally substituted heteroaryl, or haloalkyl;
R₁₄ₐ is H, haloalkyl, optionally substituted alkyl, methyl, fluoromethyl, hydroxymethyl, ethyl, isopropyl, or cyclopropyl;
R₁₅ is selected from the group consisting of H, halogen, CN, OH, NO₂, optionally substituted heteroaryl, optionally substituted aryl; optionally substituted alkyl, optionally substituted haloalkyl, optionally substituted haloalkoxy, cycloalkyl, or cycloheteroalkyl (each optionally substituted);
X is C or C═O; and

the dashed line indicates the site of attachment to the linker.

In any aspect or embodiment described herein, the linker is an poly(alkylene) glycol or of the structure —[O(CH₂)ₙ]ₘ—, wherein each n is independently 1, 2, 3, 4, 5, 6, 7 or 8, and m is an integer from 1-100 (e.g., 1, 2, 3, 4, 5, 6, 7, 8, 9, or 10).

In any aspect or embodiment described herein, the linker is selected from:

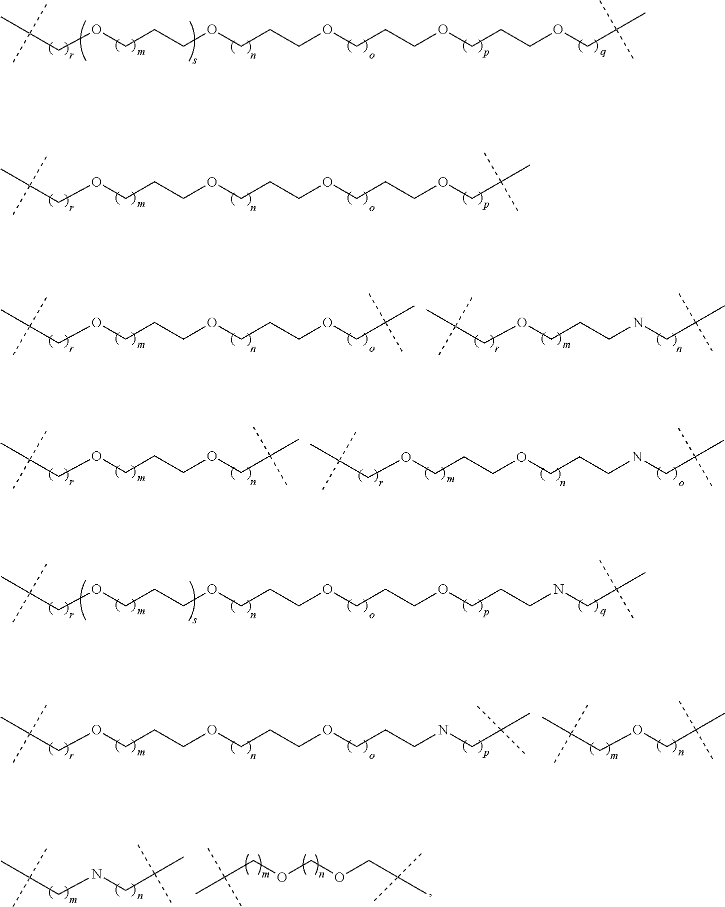

wherein each m, n, o, p, q, r, and s is independently 0, 1, 2, 3, 4, 5, 6, 7, 8, 9, 10, 11, 12, 13, 14, 15, 16, 17, 18, 19, or 20.

In any aspect or embodiment described herein, the linker is selected from:

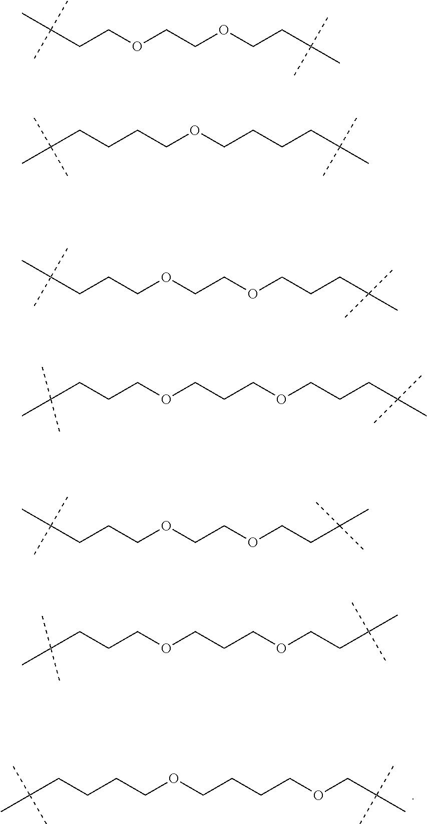

In any aspect or embodiment described herein, the linker is 9-14 (e.g., 10, 11, or 13 atoms in length) atoms in length.

In any aspect or embodiment described herein, the compound is selected from the group consisting of compound 1, 46, 48, 49, and 50.

According to another aspect, the present disclosure provides a composition comprising a pharmaceutically acceptable carrier and an effective amount of at least one compound represented by the chemical structure PTM-L-ULM for treating a disease or disorder associated with p38 (e.g., at least one of p38α, p38β, p38γ, p38δ, or a combination thereof) accumulation, aggregation, and/or activity in a subject, the method comprising administering the composition to a subject in need thereof, wherein the compound is effective in treating or ameliorating at least one symptom of the disease or disorder, and wherein:

the PTM is represented by the chemical structure:

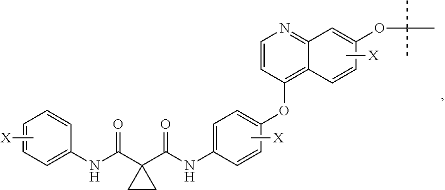

wherein each X is independently H, OMe, or F;

the linker (L) comprises a chemical structural unit represented by the formula:

-(A)_{q}-, wherein:
(A)_{q} is a group which is connected to a ULM or PTM moiety; and
   q is an integer greater than or equal to 1;
each A is independently selected from the group consisting of, a bond, CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₃₋₁₁heteocyclyl optionally substituted with 0-6 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R^{L5} groups; and
R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NH SO₂NH(C₁₋₈alkyl), NH SO₂N(C₁₋₈alkyl)₂, NH SO₂NH₂′;
   the ULM is represented by the chemical structure:

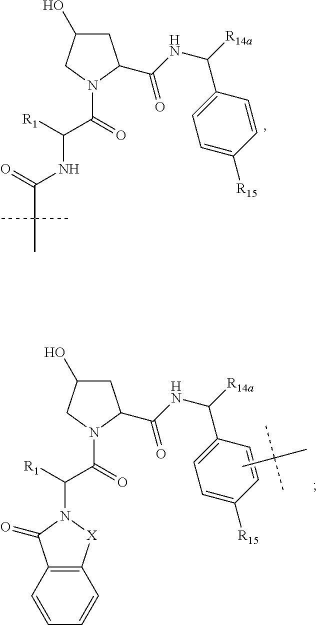

R₁ is H, ethyl, isopropyl, tert-butyl, sec-butyl, cyclopropyl, cyclobutyl, cyclopentyl, or cyclohexyl; optionally substituted alkyl, optionally substituted hydroxyalkyl, optionally substituted heteroaryl, or haloalkyl;
R₁₄ₐ is H, haloalkyl, optionally substituted alkyl, methyl, fluoromethyl, hydroxymethyl, ethyl, isopropyl, or cyclopropyl;
R₁₅ is selected from the group consisting of H, halogen, CN, OH, NO₂, optionally substituted heteroaryl, optionally substituted aryl; optionally substituted alkyl, optionally substituted haloalkyl, optionally substituted haloalkoxy, cycloalkyl, or cycloheteroalkyl (each optionally substituted);
X is C or C═O; and

the dashed line indicates the site of attachment to the linker.

In any aspect or embodiment described herein, the linker is an poly(alkylene) glycol or of the chemical structure —[O(CH₂)ₙ]ₘ—, wherein each n is independently 1, 2, 3, 4, 5, 6, 7 or 8, and m is an integer from 1-100 (e.g., 1, 2, 3, 4, 5, 6, 7, 8, 9, or 10).

In any aspect or embodiment described herein, the linker is selected from:

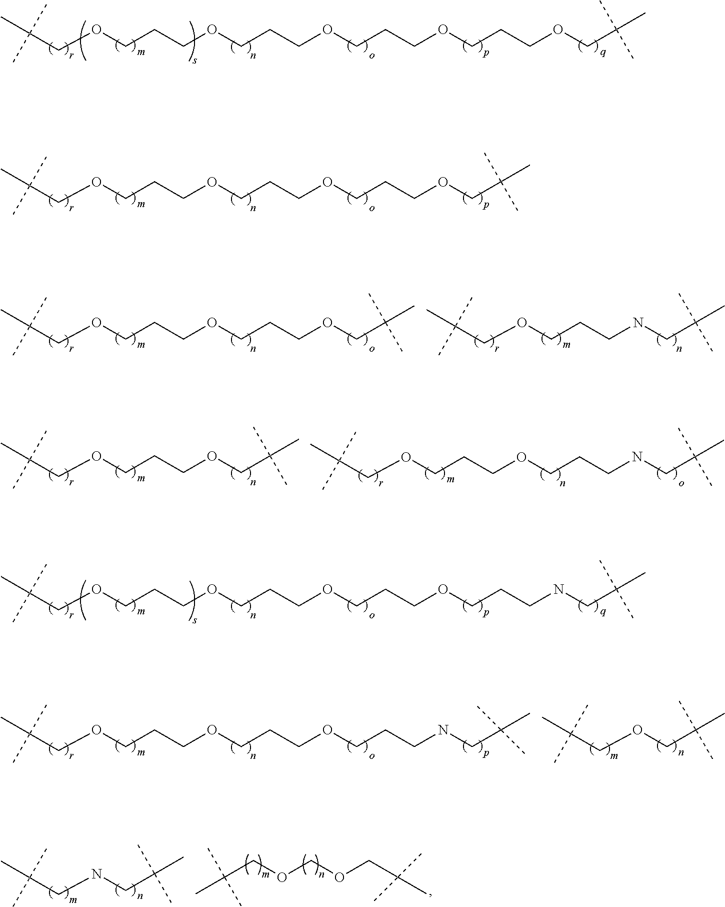

wherein each m, n, o, p, q, r, and s is independently 0, 1, 2, 3, 4, 5, 6, 7, 8, 9, 10, 11, 12, 13, 14, 15, 16, 17, 18, 19, or 20.

In any aspect or embodiment described herein, the linker is 9-14 (e.g., 10, 11, or 13 atoms in length) atoms in length.

In any aspect or embodiment described herein, the compound is selected from the group consisting of compound 1, 46, 48, 49, and 50.

In any aspect or embodiment described herein, the disease or disorder is at least one of an autoimmune or inflammatory disease or disorder, cancer, cardiovascular disease or disorder, a neurological disease or disorder, or a combination thereof.

The composition of any of claims 29-32, wherein: (1) the autoimmune disease or disorder is at least one of: rheumatoid arthritis, cerebral ischemia, muscular dystrophy, diabetes mellitus, Crohn's disease, psoriasis, ankylosing spondylitis, chronic asthma, chronic pulmonary obstructive disorder, or a combination thereof; (2) the cancer is at least one of: gastric cancer, non-small cell lung cancer, squamous-cell carcinoma, basal cell carcinoma, adenocarcinoma, hepatocellular carcinomas, advanced hepatocellular carcinoma, renal cell carcinomas, and papillary renal cell carcinoma; cancer of the bladder, bowel, breast, cervix, colon, esophagus, head, kidney, liver, lung, neck, ovary, pancreas, prostate, and stomach; leukemia; benign and malignant lymphomas, particularly Burkitt's lymphoma and Non-Hodgkin's lymphoma; myeloma; multiple myeloma, benign and malignant melanomas; myeloproliferative diseases; sarcomas, Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas; T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, B-cell chronic lymphatic leukemia, Philadelphia chromosome positive ALL, Philadelphia chromosome positive CML; or combinations thereof; (3) the cardiovascular disease or disorder is at least one of: ischemia, ischemia-reperfusion, or a combination thereof; (4) the neurological disease or disorder if at least one of: Parkinson's disease, Alzheimer's disease, multiple sclerosis, amyotrophic lateral sclerosis (ALS), chronic inflammatory demyelinating polyradiculoneuropathy, fibromyalgia, polymyositis, or a combination thereof; or (5) a combination thereof.

In certain embodiments, the description provides following exemplary c-Met PROTAC molecules (e.g., compounds 1-50 of Table 1), including salts, prodrugs, polymorphs, analogs, derivatives, and deuterated forms thereof.

In certain embodiments, the description providing the following exemplary p38 (e.g., at least one of p38α, p38β, p38γ, p38δ, or a combination thereof) PROTAC molecules (e.g., compounds 1-50 of Table 1), including salts, prodrugs, polymorphs, analogs, derivatives, and deuterated forms thereof.

### Examples

Protein Degradation Examination

Protocol of the cellular assay of target protein degradation (MDA-MB-231 cells, ELISA).

For detection Cell Signaling PathScan Sandwich ELISA Catalog#12850 was used. MDA-MB-231 cells and p38α cells were cultured in ATCC DMEM+ATCC FBS and plated 40,000/well 100 μl/well in RPMI P/S with 5% CSS Omega (bovine) serum into a 96 well plate. The cells were grown for a minimum of 3 days, dosed with compounds in 0.1% DMSO (diluted with 5% CSS) and incubated with aspiration for 4 hours. 100 μl of 1× Cell Signaling lysis buffer #9803 (36 mL dH₂O+4 mL Cell Signaling lysis buffer) was added. The incubation was placed on cold room shaker for 10 minutes at speed 8-9.5 μl to 100 μL of Diluent was transferred to ELISA plate (0.15 μg/mL-0.075 μg/mL) and stored at 4° C. overnight on cold room shaker speed 5 (gentle swirl) and then shaken next morning at 37° C. for 30 minutes. The preparation was washed 4×200 μl with ELISA wash buffer and aspirated with eight-channel aspirator. 100 μl/well of target protein detection antibody was added after, which the preparation was covered and shaken at 37° C. for 1 hour. 100 μl TMB was added, and the mixture was shaken for 5 min while under observation. When TMB turned light blue, 100 μl of Stop solution was added, and the mixture was shaken and read at 450 nM. Also read at 562 nm for background subtraction.

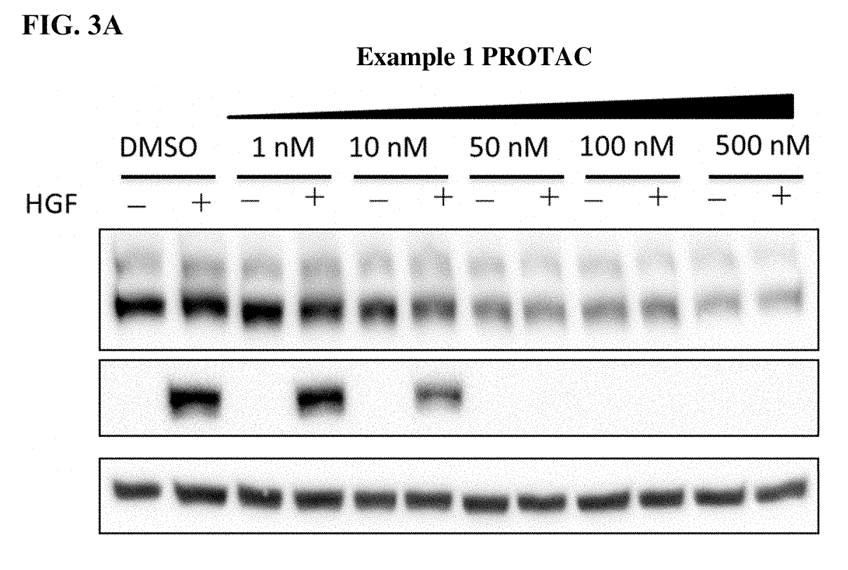

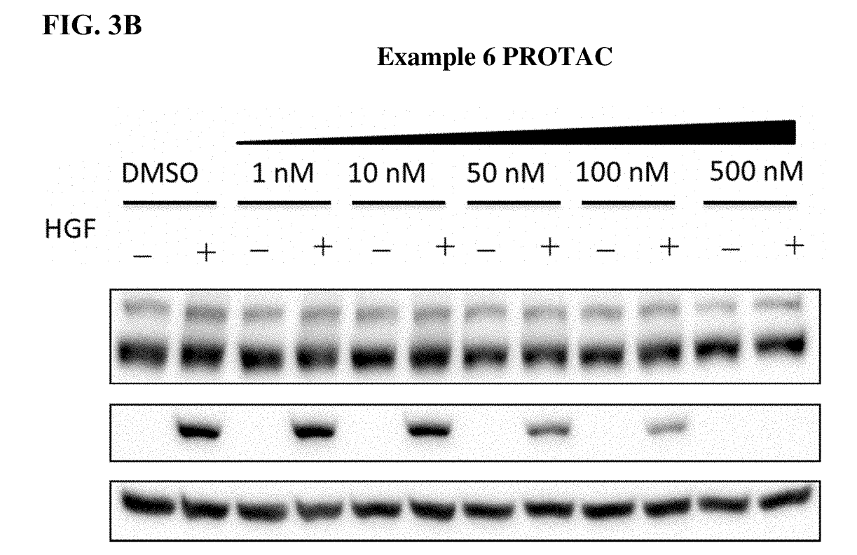

The PROTACs of Table 1 (see FIG. 2) demonstrated c-Met protein degradation when tested under the conditions described above. c-Met, p38α, and p38δ protein degradation for Examples 45-50 are shown in Table 2. Dose Response curves for exemplary PROTACS 1 and 6 is shown in FIGS. 3A and 3B. Each of the PROTACS demonstrated significant targeted degradation with Example 1 being slightly more effective.

TABLE 2
Target protein degradation Determination for Examples 45-50
Exemplary

| | cMET DC50/ | | p38α DC50/ | | | |
|---|---|---|---|---|---|---|
| | nM [MDA-MB- | | nM [MDA-MB- | | p38δDC50/nM | |
| | 231] | Dmax | 231] | Dmax | [MDA-MB-231] | Dmax |
| Ex. 45 | n/a | n/a | >5000 | 27% | >5000 | 43% |
| Ex. 46 | n/a | n/a | >5000 | 33% | 84.9 | 93% |
| Ex. 47 | n/a | n/a | >5000 | 12% | >5000 | 23% |
| Ex. 48 | n/a | 44% | 18.8 | 99% | 91.7 | 91% |
| Ex. 49 | n/a | 49% | 40.1 | 91% | 51.4 | 81% |
| Ex. 50 | 36 | 88% | 9.5 | 99 | >5000 | 18% |

Western Blot Examination of Protein Degradation.

MDA-MB-231 cells (1-1.5×10⁶) were treated for 24 hours with the indicated compounds solubilized in DMSO. The cells were collected at 300 g for 3 minutes. The cells were then lysed in lysis buffer (25 mM Tris, 1% Triton, 0.25% deoxycholic acid) with Roche protease inhibitor complete cocktail and phosphatase inhibitors (10 mM sodium fluoride, 10 mM sodium pyrophosphate, 1 mM sodium orthovanadate and 20 mM β-glycerophosphate). The total protein concentrations were determined by Pierce BCA Protein Assay and 30-50 μg of protein was loaded onto 10% Tris-Glycine gels. After standard gel electrophoresis, the separated proteins were transferred to nitrocellulose by wet transfer. The immunoblots were then processed by standard procedures and incubated with the respective antibodies. Band intensities were quantified by Bio-Rad's Image Lab software.

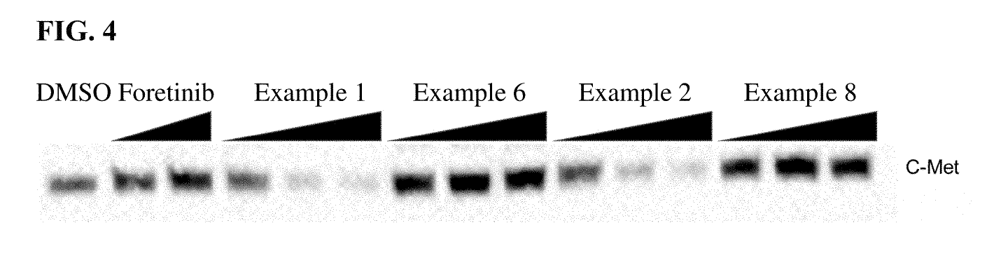

As shown in FIG. 4, the exemplary VHL PROTAC Example 1 and Cereblon PROTAC Example 2 demonstrated significant protein degradation of c-Met.

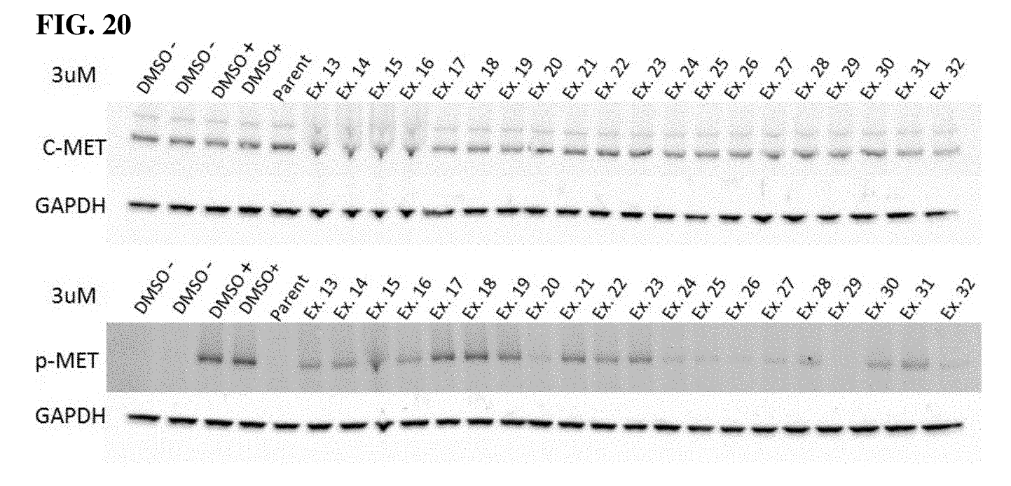

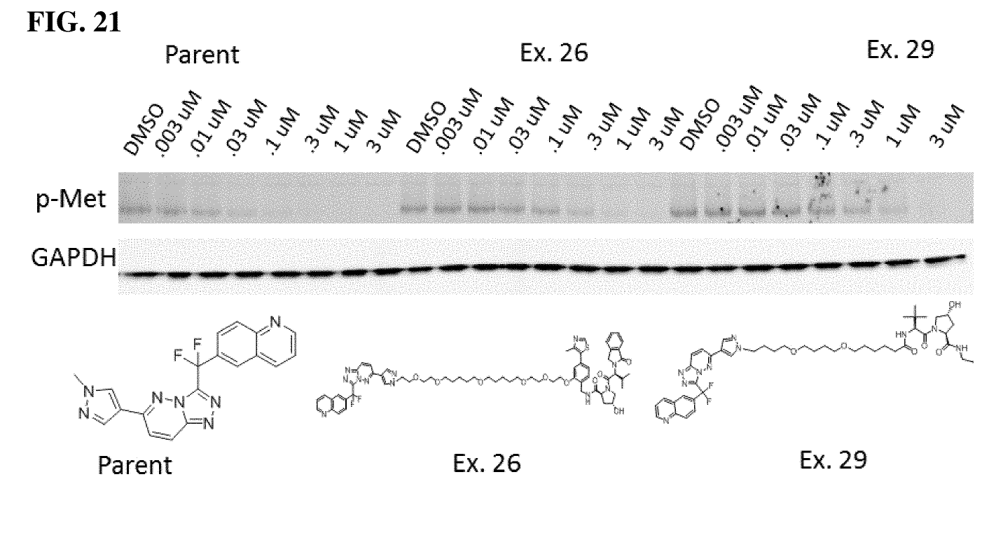

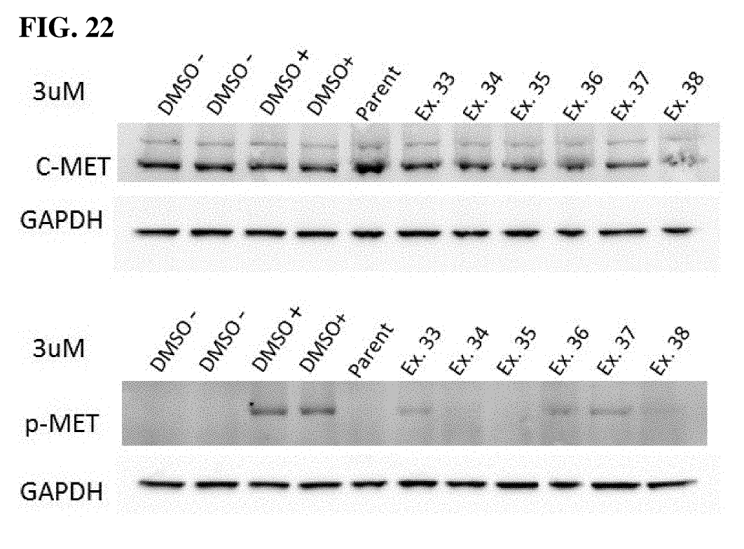

The ability of exemplary compounds to bind and inhibit c-MET was examined via the westernblot protocol described above. Compounds that are able to enter the cell, bind and inhibit c-MET will have reduced phosphorylation. Thus, as demonstrated in FIGS. 20-22, exemplary compounds of the present disclosure were able enter the cells and to bind and inhibit c-MET.

Pharmacokinetics and Protein Binding Examination

Pharmacokinetics of Intraperitoneal or Intravenous Injection of Example 1 or Example 2.

The pharmacokinetics of Example 1 and 2 was examined for intraperitoneal and intravenous administration. The desired serial concentrations of working solutions were achieved by diluting stock solution (1 mg/mL in DMSO) of analyte with methanol. In particular, 20 μL of working solutions (1, 2, 5, 10, 50, 100, 500, 1000, 2000 ng/mL) were added to 20 μL of the blank CD1 mice plasma to achieve calibration standards of 1-2000 ng/mL (1, 2, 5, 10, 50, 100, 500, 1000, 2000 ng/mL) in a total volume of 40 pt. Dose of IV and IP were diluted by 1000 fold and 10000 with methanol, 20 μL of the diluted dose solutions were added to 20 μL of the blank CD1 mice plasma to achieve dose samples. Next, 40 μL standards and 40 μL dose samples were added to 20 μL of assay Internal Standard (100 ng/mL) and 200 μL of acetonitrile for precipitating protein respectively. Then the samples were vortexed for 30 seconds. After centrifugation at 4 degree Celsius, 4000 rpm for 15 min, the supernatant was diluted 3 times with water. Then, 10 μL of the diluted supernatant was injected into the LC/MS/MS system for quantitative analysis.

Examples 1 and 2 were administered intravenously (5 second injection of 10% HP-b-CD and 40 mM sodium acetate and 40 mM NaCl in water, pH4.5) or intraperitoneally (5% EtOH and 5% Solutol HS15 in D5W(ESD-2)) to 6-8 week old male CD1 mice (3 mice per treatment group for a total of 12 mice) at the time points indicated below in Table 3. The mice had free access to food and water.

TABLE 3
Pharmacokinetics study design for Examples 1 and Example 2

| | | Dosing | Dosing | Dosing |
|---|---|---|---|---|
| Route of | | Level | Concentration | Volume |
| Administration | Time Points | (mg/kg) | (mg/mL) | (mL/kg) |
| IV | 0.033 h, 0.083 h, | 1.0 | 0.2 | 5 |
| | 0.25 h, 0.5 h, 1 h, | | | |
| | 2 h, 4 h, 8 h, 24 h | | | |
| IP | 0.25 h, 0.5 h, 1 h, | 10 | 1.0 | 10 |
| | 2 h, 4 h, 8 h, 24 h | | | |

Dorsal metatarsal vein samples (30 μL), except that the final sample was acquired via a heart puncture, were taken from the mice at the same the administration time points as shown in Table 2.

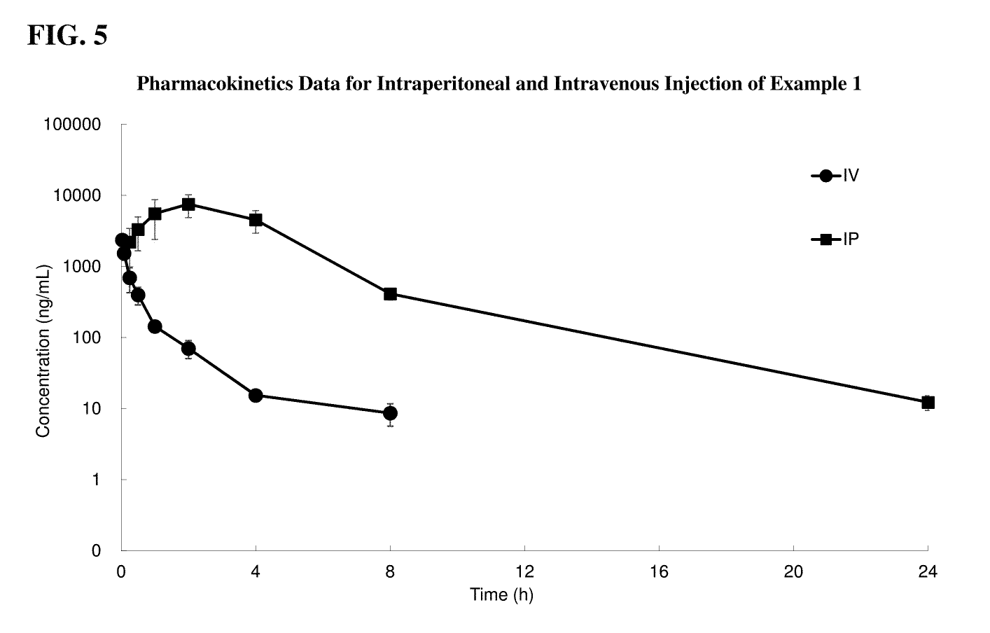

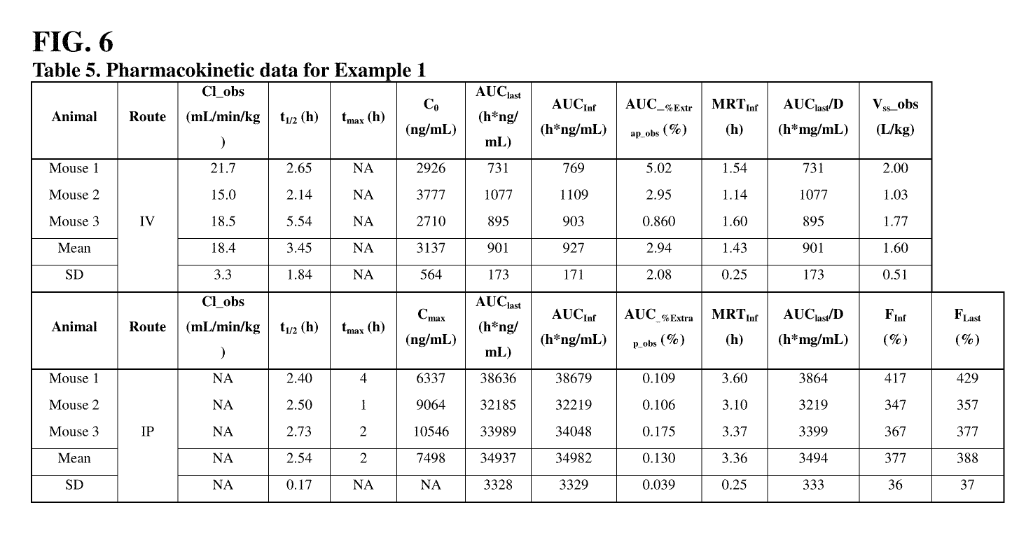

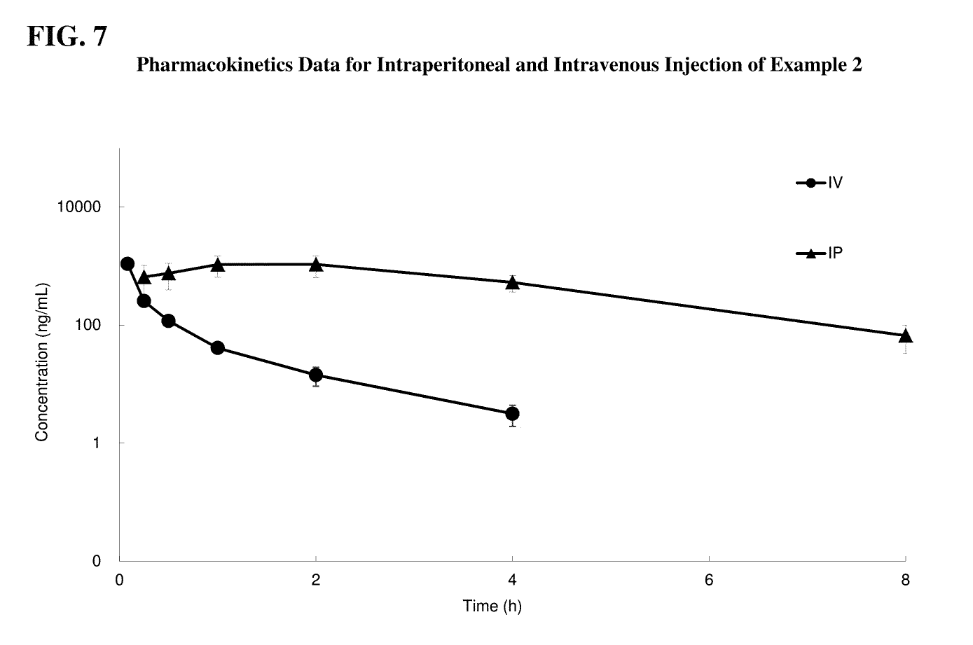

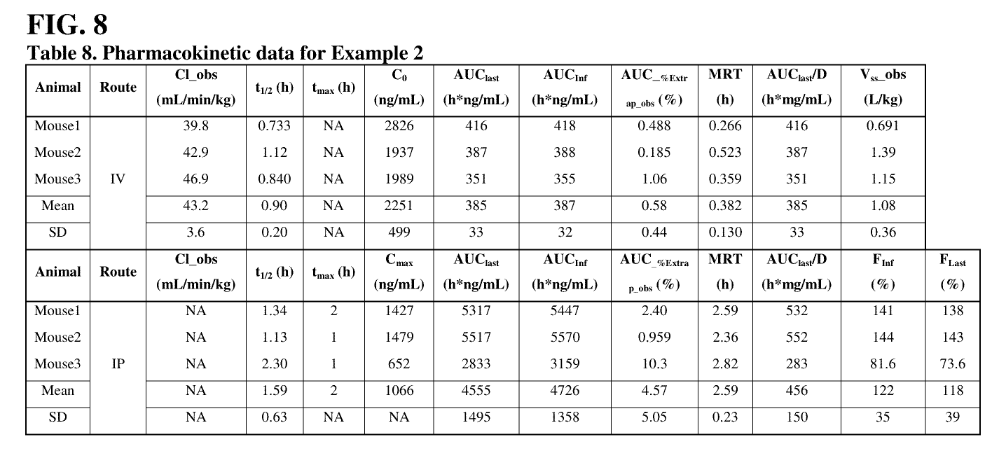

The bioanalytical assay using an internal standward was performed on the samples as shown in Table 4 below. The data for Example 1 is shown below in FIG. 5 and Tables 5 (FIG. 6), 6 and 7. The data for Example 2 is shown in FIG. 7 and Tables 8 (FIG. 8), 9, and 10.

TABLE 4
Bioanalysis of the pharmacokinetic examination of Example 1
and Example 2

| | | |
|---|---|---|
| Bioanalytical | HPLC | Instrument: SHIMADZU (LC-30A D; |
| Assay: | | DGU-20A5R; CBM-20A; SIL-30AC; |
| | | CTO-30A); Rack Changer II |
| | MS | LCMS-8050 instrument (Serial NO. |
| | | O10835400523 AE) |
| | Column | Phenomenex Kinetex 5u C18 |
| | | (50 * 2.1 mm) |
| | Mobile Phase | 95% Water (0.1% Formic Acid) and |
| | | 95% Acetonitrile (0.1% Formic Acid) |
| | Quantification | Internal Standard Method |

TABLE 6
Pharmacokinetic data for intravenously injected Example 1

| | Conc. (ng/mL) | | | Mean | SD |
|---|---|---|---|---|---|
| Time (h) | Mouse 1 | Mouse 2 | Mouse 3 | (ng/mL) | (ng/mL) |
| 0.033 | 2050 | 2819 | 2190 | 2353 | 410 |
| 0.083 | 1196 | 1810 | 1586 | 1531 | 310 |
| 0.25 | 491 | 991 | 590 | 691 | 264 |
| 0.5 | 280 | 497 | 413 | 397 | 109 |
| 1 | 142 | 135 | 153 | 143 | 9 |
| 2 | 55.5 | 92.9 | 62.3 | 70.3 | 19.9 |
| 4 | 16.8 | 15.4 | 13.9 | 15.4 | 1.5 |
| 8 | 10.1 | 10.6 | 5.20 | 8.6 | 3.0 |
| 24 | BLOQ | BLOQ | 0.971 | NA | NA |

TABLE 7
Pharmacokinetic data for intraperitoneally injected Example 1

| | Conc. (ng/mL) | | | Mean | SD |
|---|---|---|---|---|---|
| Time (h) | Mouse 1 | Mouse 2 | Mouse 3 | (ng/mL) | (ng/mL) |
| 0.25 | 3417 | 2182 | 990 | 2196 | 1214 |
| 0.5 | 5209 | 2502 | 2209 | 3307 | 1654 |
| 1 | 4467 | 9064 | 3051 | 5528 | 3143 |
| 2 | 5740 | 6208 | 10546 | 7498 | 2650 |
| 4 | 6337 | 3742 | 3465 | 4515 | 1584 |
| 8 | 476 | 329 | 429 | 411 | 75 |
| 24 | 12.2 | 9.43 | 15.1 | 12.3 | 2.9 |

TABLE 9
Pharmacokinetic data for intravenously injected Example 2

| | Conc. (ng/mL) | | | Mean | SD |
|---|---|---|---|---|---|
| Time (h) | Mouse 1 | Mouse 2 | Mouse 3 | (ng/mL) | (ng/mL) |
| 0.083 | 1283 | 997 | 987 | 1089 | 168 |
| 0.25 | 262 | 262 | 241 | 255 | 12 |
| 0.5 | 110 | 129 | 114 | 117 | 10 |
| 1 | 35.4 | 48.1 | 38.8 | 40.8 | 6.5 |
| 2 | 9.62 | 19.5 | 13.4 | 14.2 | 5.0 |
| 4 | 1.93 | 4.39 | 3.11 | 3.14 | 1.23 |
| 8 | BLOQ | 0.447 | BLOQ | NA | NA |
| 24 | BLOQ | BLOQ | BLOQ | NA | NA |

TABLE 10
Pharmacokinetic data for intraperitoneally injected Example 2

| | Conc. (ng/mL) | | | Mean | SD |
|---|---|---|---|---|---|
| Time (h) | Mouse 1 | Mouse 2 | Mouse 3 | (ng/mL) | (ng/mL) |
| 0.25 | 519 | 1075 | 363 | 652 | 374 |
| 0.5 | 721 | 1129 | 411 | 754 | 360 |
| 1 | 1042 | 1479 | 652 | 1058 | 413 |
| 2 | 1427 | 1173 | 596 | 1066 | 426 |
| 4 | 620 | 630 | 336 | 529 | 167 |
| 8 | 67.3 | 32.9 | 98.4 | 66.2 | 32.8 |
| 24 | BLOQ | BLOQ | BLOQ | NA | NA |

The IV and IP routes of administration were effective at increasing plasma levels of Example 1 and Example 2. As can be seen in the data, when an order of magnitude more is adminstered via IP, there is a concomitant increase in Cmax and AUC.

In Vitro Kinase Binding Affinity Determination for Example 1 and Example 2.

PROTAC in vitro binding affinities (Kd) to c-Met was determined using the KINOMEscan™ platform (DiscoverRx Corporation). The compounds were solubilized in DMSO and sent to DiscoverRx Corporation as 10 μM stock solutions.

Example 1 and Example 2 were screened at a 1000 nM concentration, and the results of the binding interactions are reported in “% Ctrl”, where a lower number indicates stronger binding. % Ctrl=[(test compound signal−positive control signal)/(negative control signal−positive control signal)]×100, wherein the negative control was DMSO. As shown in the data of Table 11, below, Example 1 and Example 2 demonstrated strong binding to MET.

TABLE 11
KINOMEscan ™ Data for Example 1 and Example 2

| Target Protein | Foretinib | Example 1 | Example 2 |
|---|---|---|---|
| MET | 0.65 | 0.75 | 1.4 |
| MET (M1250T) | 1.8 | 6.0 | 7.2 |
| MET (Y1235D) | 13 | 8.2 | 14 |
| MAK | 55 | 92 | 94 |

Examination of p38α and p38δ Protein Degradation

Owing to the highly modular nature of PROTACs, recent work has underscored the importance of PTM (warhead) selection and E3 ligase choice in the PROTAC-induced degradation of specific protein targets. In particular, it has been shown that while dasatinib-based CRBN-recruiting PROTACs could degrade both c-Abl and BCR-Abl, dasatinib-based VHL-recruiting PROTACs could degrade only c-Abl. However, when using a bosutinib PTM, CRBN-recruiting PROTACs were able to degrade both c-Abl and BCR-Abl, whereas corresponding VHL-recruiting PROTACs could not degrade either protein target. Additionally, VHL- and CRBN-recruiting PROTACs based on the BET-targeting triazolodiazepine JQ1 show potent degradation of BRD2, BRD3, and BRD4 and early characterization of thienopyrininone-based CRBN-recruiting PROTACs have demonstrated the additional ability to degrade BRD4, BRD7, and BRD9 proteins.

This example was contextualized by the recent publication of a BRD4:PROTAC:VHL crystal structure, in addition to two studies that surveyed proteome-wide degradation using PROTACs based on promiscuous PTMs. The ternary complex crystal structure revealed key protein-protein interactions (PPIs) between VHL and BRD4 as well as mutual interactions with the PROTAC linker region. On a proteomic-scale, one study demonstrated that ternary complex formation is a far more predictive driving force behind PROTAC-induced substrate degradation than is target:PROTAC binding affinity. Using foretinib as a PTM, it has been shown that VHL- and CRBN-utilizing PROTACs resulted in different degradation profiles, but each degradation profile was, similarly, only a subset of the kinases to which the PROTAC bound. These degraded substrate proteins included even those with only weak affinity for the PROTAC itself, thus demonstrating how compensatory PPIs afforded by the substrate:PROTAC:ligase interface provide an added layer of selectivity and affinity for some target proteins.

The inventors hypothesized and examiner herein that it would be possible to generate PROTACs that selectively degrade closely-related proteins using only a single warhead (PTM) and E3 ligase. Isoforms of the p38 MAPK family are examined because of their lack of isoform-selective chemical probes and their vast and varied role in neurodegenerative diseases, cardiovascular disease, and cancer.

Here, p38α- and p38δ-selective PROTACs were developed based on a single PTM (foretinib) and E3 ligase (VHL). By fixing a pan-active compound to one end of a PROTAC, differences in linker length and orientation of the recruited VHL to bias the degradation outcome of closely-related kinase isoforms were examined. It was found that the formation of a ternary complex is a necessary but not sufficient step in discriminating between substrate proteins and determined that the affinities of these interactions are highly indicative of cellular outcomes.

Identification of p38 Isoform-Selective PROTACs

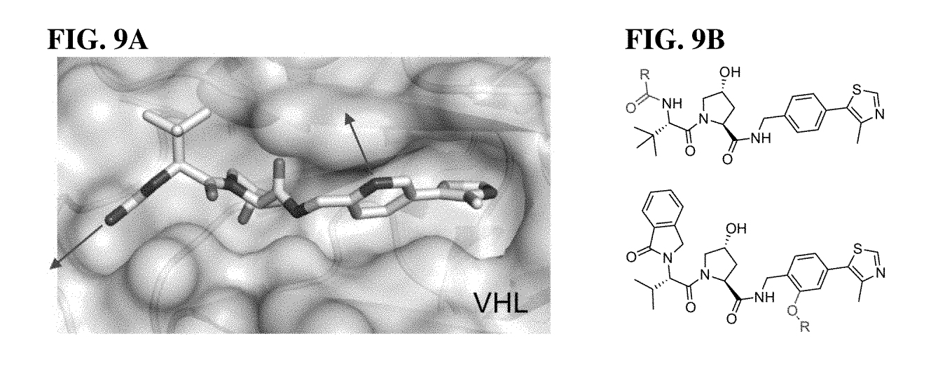

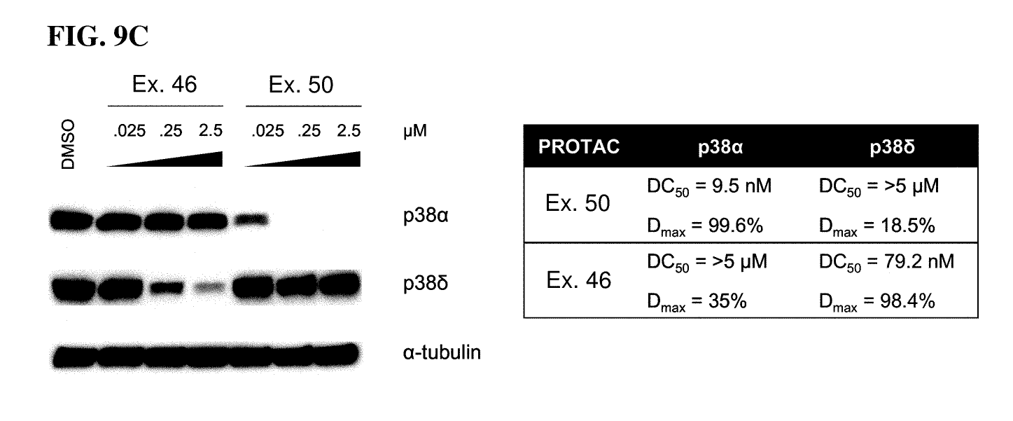

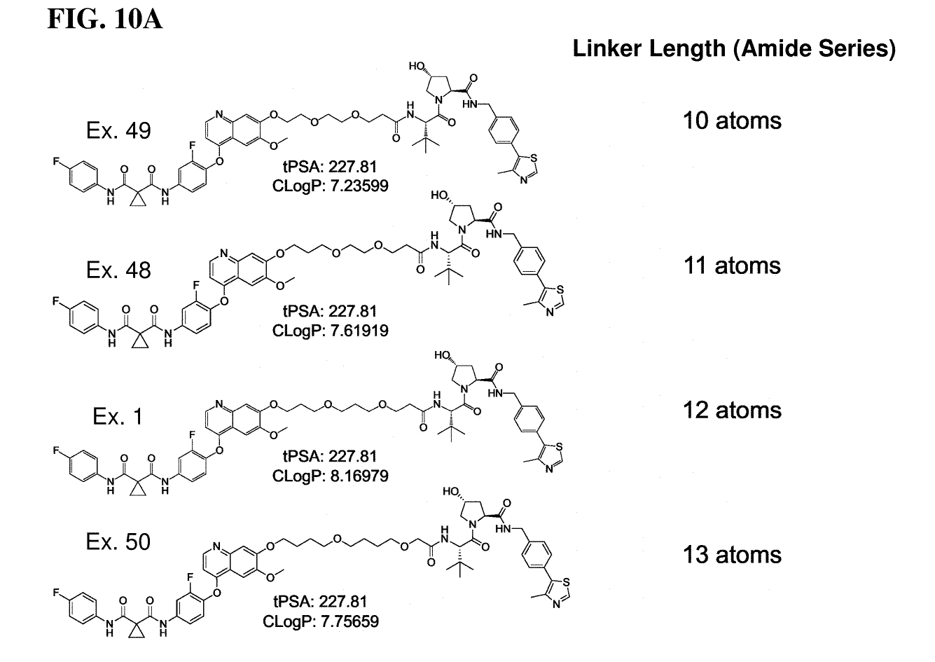

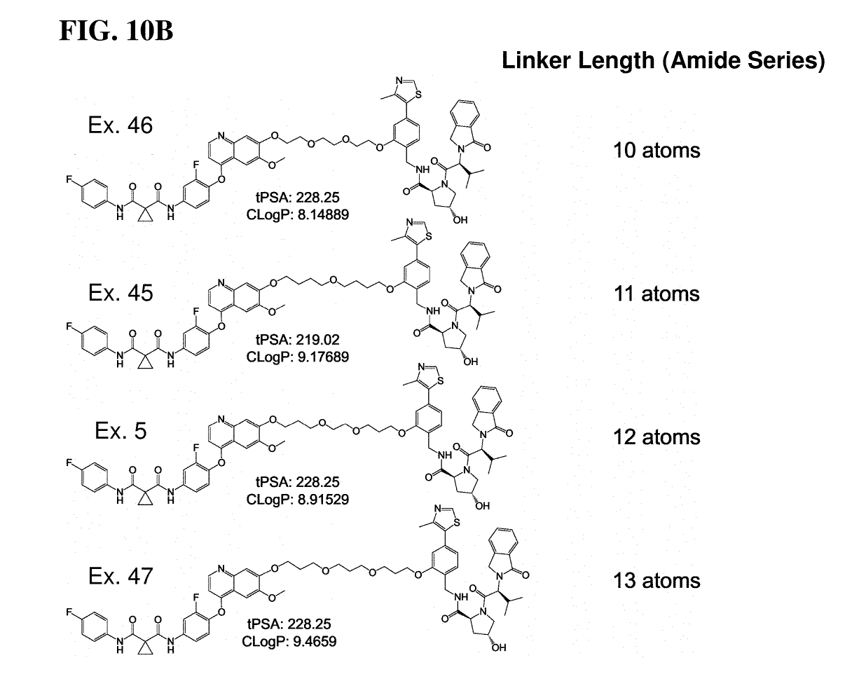

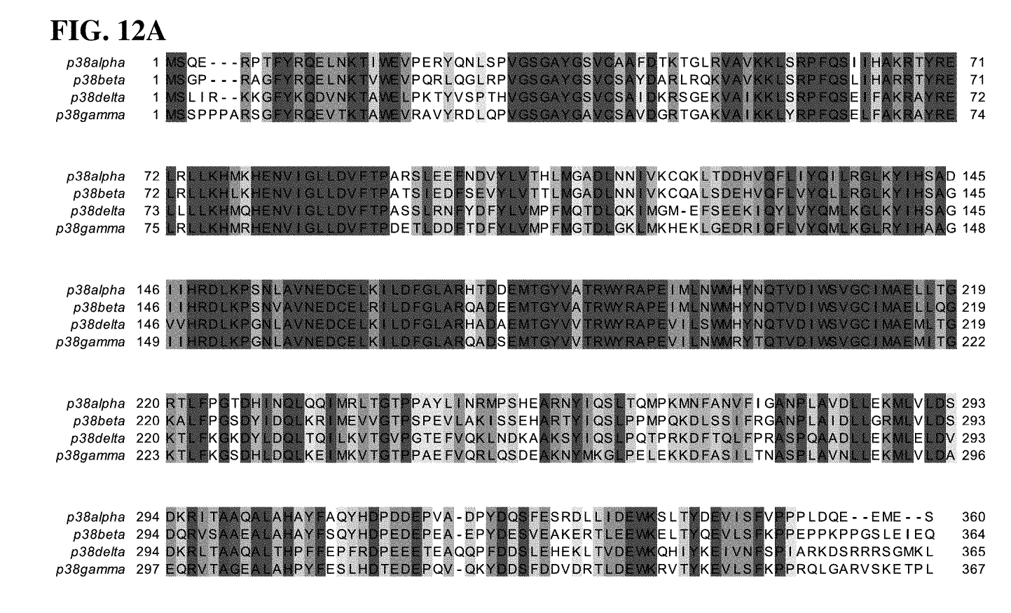

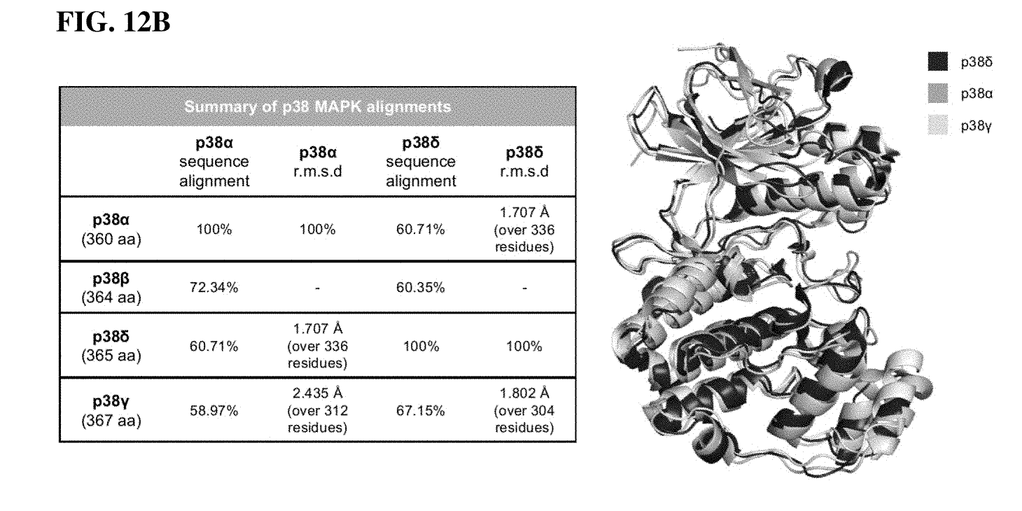

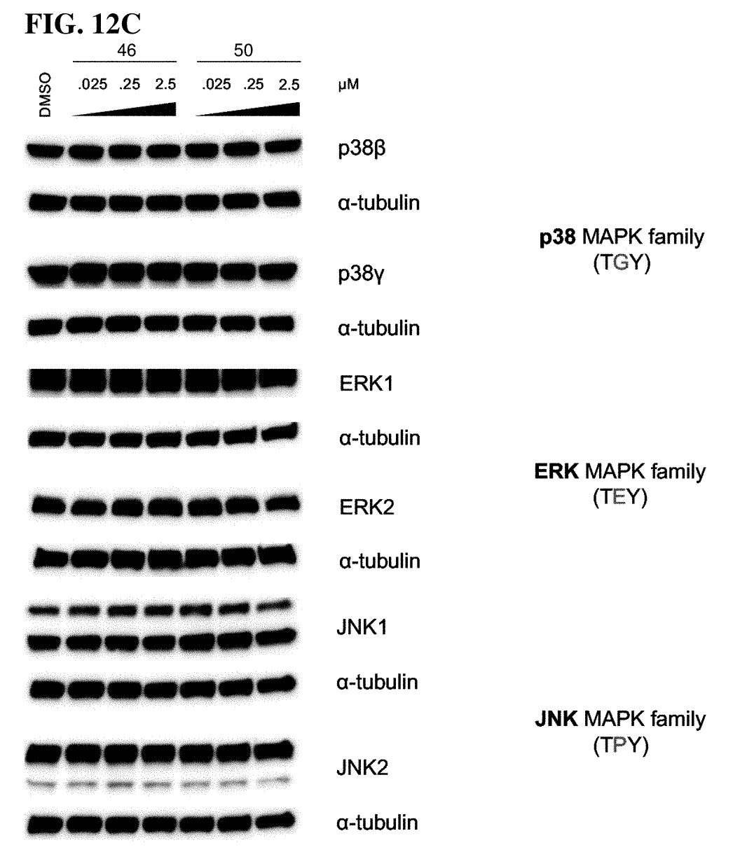

To address the mechanistic questions surrounding PROTAC substrate specificity was assessed by synthesizing eight foretinib-based PROTACs that vary by linker length and by orientation of the VHL-recruiting molecule. PROTACs 1 and 48-50 (termed the ‘amide series’) were constructed using the previously-characterized “left-hand side” amide as a connection point for linker attachment to the VHL ligand, while PROTACs 5 and 45-47 (termed the ‘phenyl series’) incorporated an under-explored “right-hand side” phenyl ring attachment point of the linker to the VHL ligand (FIGS. 9A and 9B). Both PROTAC series utilized the same phenyl ether linkage attachment from the solvent-exposed region of the foretinib PTM and used linker lengths of 10-, 11-, 12-, and 13-atoms (FIGS. 10A, 10B, 11A, and 11B, 12A and 12B). PROTACs synthesized with different linker lengths, using two different points of attachment to the VHL-recruiting ligand promote distinct E3 ligase interfaces with a given p38 isoform, thereby enabling potential degradation selectivity. Indeed, this strategy of differential orientation of recruited VHL coupled with different PROTAC linker lengths enabled the identification of two PROTACs that each selectively degrade only a single p38 MAPK isoform in MDA-MB-231 cells (FIG. 9C). The examplary compound 50 and exemplary compound 46 result in potent and selective degradation of p38α and p38δ, respectively, without an observable “hook effect” within the tested dose range. Exemplary compound 50 degrades p38α with a DC₅₀ (concentration at which half-maximal degradation is observed) of 9.5±1.0 nM and measurable Dₘₐₓ (maximum percentage of degradation achieved) of 99.6%, where as exemplary compound 46 degrades p38δ with a DC₅₀ of 79.2±28 nM and Dₘₐₓ of 98.4% (Table 12 and 13). Crucially, the DC₅₀ for exemplary compound 50 on p38δ is >5 μM, as is the DC₅₀ of exemplary compound 46 for p38α. Additionally, neither exemplary PROTAC degrades either of the two remaining p38 MAPK isoforms (β and γ), despite high sequence and structural conservation. Moreover, neither PROTAC degrades related MAPKs ERK1, ERK2, JNK1, and JNK2 (FIGS. 12A, 12B, and 12C), demonstrating the profound isoform selectivity of our two lead PROTACs.

TABLE 12
Exemplary p38δ protein degradation data for exemplary compounds 46
and 50
Summary of binary (PROTAC:p38δ or PROTAC:VHL) and turnary
(p38δ:PROTAC:VHL) affinity and kinetic measurements*

| | Ex. 46 | | Ex. 50 | |
|---|---|---|---|---|
| PROTAC:p38δ | 550 | nM | 500 | nM |
| K_{d} | | | | |
| PROTAC:VHL | 3500 | nM | 5000 | nM |
| K_{d} | | | | |
| p38δ:PROTAC:VHL | 436 | nM | 1200 | nM |
| K_{d} | | | | |
| p38δ:PROTAC:VHL | 0.0175 | s⁻¹ | 0.072 | s⁻¹ |
| k_{off} | | | | |
| p38δ:PROTAC:VHL | 39621 | M⁻¹ s⁻¹ | 70119 | M⁻¹ s⁻¹ |
| kₒₙ | | | | |
| p38δ:PROTAC:VHL | 38 | s | 8 | s |
| t_{1/2} | | | | |
| p38δ:PROTAC:VHL | 330 | s | 80 | s |
| residence time | | | | |

*The last protein listed in each at above represents that which was immobilized on the SPR instrument. See Materials and Methods for further descriptions.

TABLE 13
Exemplary
Summary of p38α/p38δ degradation

| | p38α | p38δ |
|---|---|---|
| Amide PROTAC series | | |
| Ex. 49 | DC₅₀ = 40.1 ± 7.7 nM | DC₅₀ = 51.4 ± 7.4 nM |
| (10 atoms) | Dₘₐₓ = 91% | Dₘₐₓ = 81% |
| Ex. 48 | DC₅₀ = 18.8 ± 2.7 nM | DC₅₀ = 91.7 ± 18 nM |
| (11 atoms) | Dₘₐₓ = 99.4% | Dₘₐₓ = 91% |
| Ex. 1 | DC₅₀ = 230 ± 47 nM | DC₅₀ = >5 μM |
| (12 atoms) | Dₘₐₓ = 83% | Dₘₐₓ = 42% |
| Ex. 50 | DC₅₀ = 9.5 ± 1.0 nM | DC₅₀ = >5 μM |
| (13 atoms) | Dₘₐₓ = 99.6% | Dₘₐₓ = 18.5% |
| Phenyl PROTAC series | | |
| Ex. 46 | DC₅₀ = >5 μM | DC₅₀ = 79.2 ± 28 nM |
| (10 atoms) | Dₘₐₓ = 35% | Dₘₐₓ = 98.4% |
| Ex. 45 | DC₅₀ = >5 μM | DC₅₀ = >5 μM |
| (11 atoms) | Dₘₐₓ = 25% | Dₘₐₓ = 44% |
| Ex. 5 | DC₅₀ = 5 μM | DC₅₀ = 4.9 μM |
| (12 atoms) | Dₘₐₓ = 17% | Dₘₐₓ = 52% |
| Ex. 47 | DC₅₀ = >5 μM | DC₅₀ = >5 μM |
| (13 atoms) | Dₘₐₓ = 20% | Dₘₐₓ = 33% |

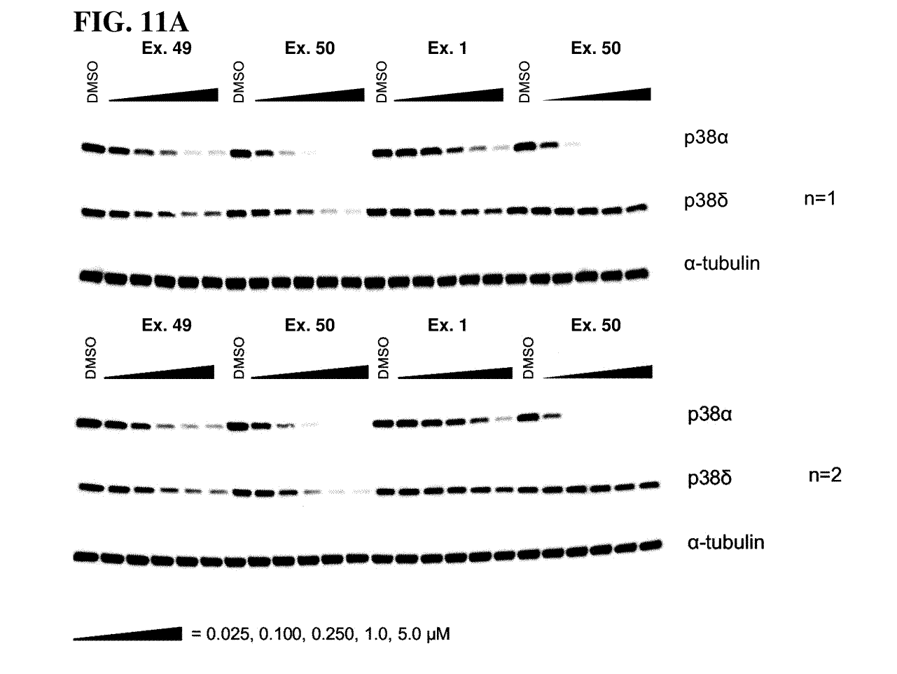

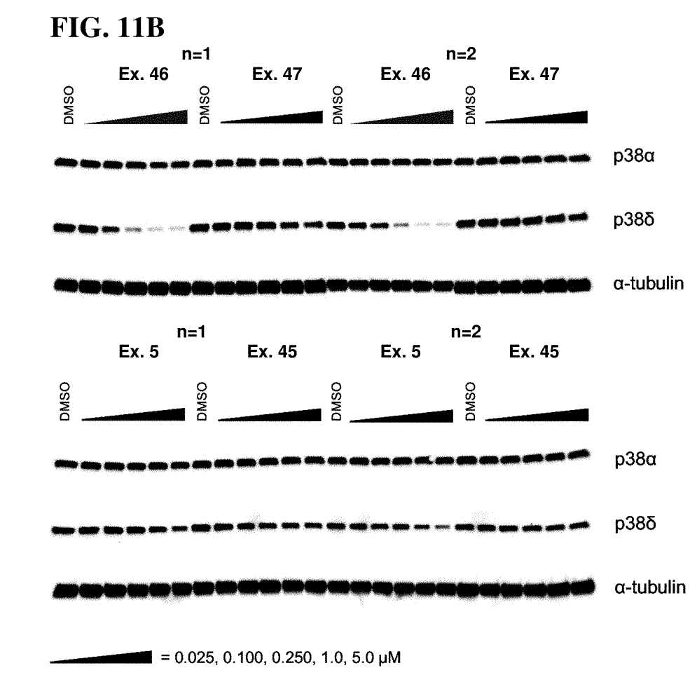

Interestingly, a 10-atom linker and a 11-atom linker amide series PROTACs (exemplary compounds 49 and 48) were identified that degrade both p38α and p38δ with DC₅₀<100 nM (Table 13, FIGS. 10, 11A, and 11B). However, this promiscuous p38 degradation by the amide series became drastically more p38α isoform-selective with the 12-atom (exemplary compound 1) and 13-atom (exemplary compound 50) linker PROTACs. By contrast, the only phenyl series PROTAC that demonstrated appreciable p38 degradation is the 10-atom exemplary compound 46, highlighting large degradation outcome differences between the two PROTAC series (amide and phenyl) in addition to distinctions in the “optimal” linker length required to achieve selective degradation of a given p38 isoform.

Exemplary Compounds 46 and 50 Achieve Selective p38 Isoform Degradation in a Manner that is Consistent with PROTAC Action

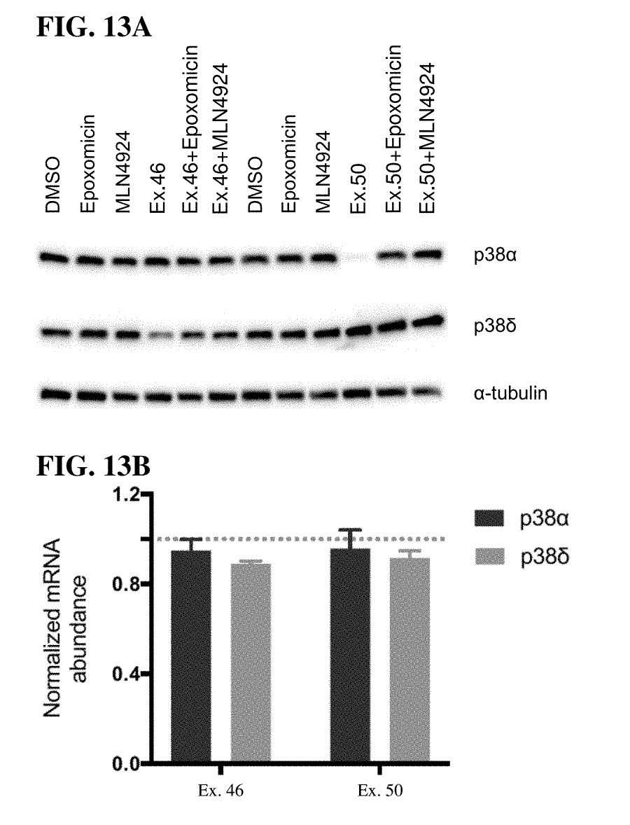

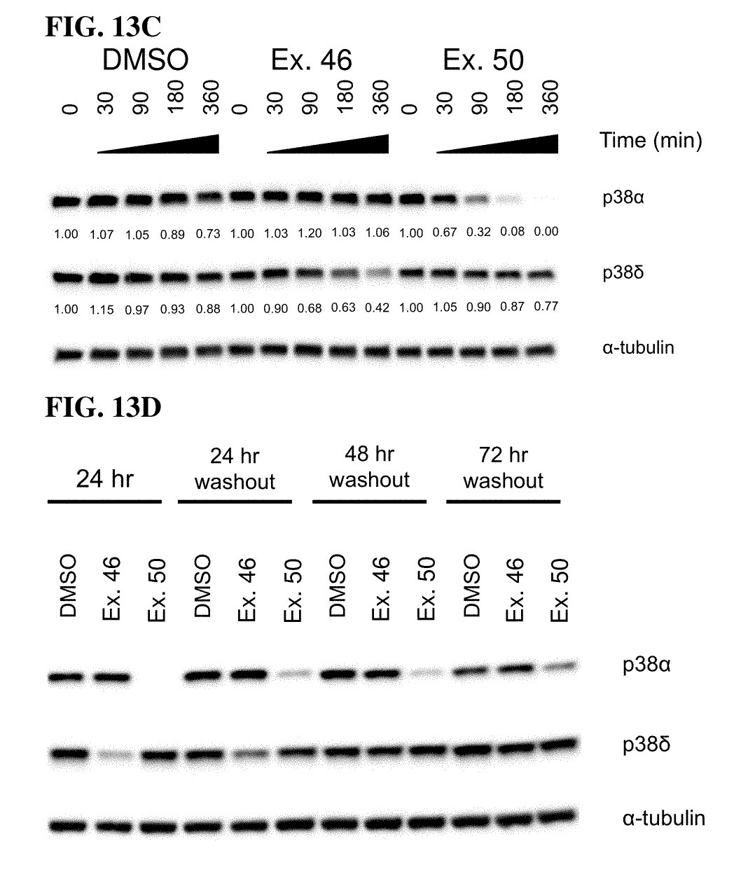

Given this intriguing isoform-selective loss of p38 observed in response to PROTAC treatment, it was examined whether the isoform-selective loss of p38 was due only to post-translational degradation. PROTACs function through degradation of existing target proteins via the ubiquitin proteasome pathway, rather than by suppression of target protein synthesis (as is the case with nucleic acid-based knockdown strategies). Accordingly, it was investigated whether exemplary compounds 50- and 46-mediated downregulation of their respective p38 targets in MDA-MB-231 cells proceeds in a manner that is consistent with the mechanism of action of PROTACs. Cells treated with 250 nM exemplary compound 50 or exemplary compound 46 for 6 hours resulted in downregulation of p38α and p38δ, respectively, which is completely rescued when cells were pre-treated with either 1 μM epoxomicin (proteasome inhibitor) or 1 μM MLN4924 (NEDD8-activating enzyme inhibitor), indicating that these PROTACs depend on the proteasome and ubiquitination cascade (i.e. neddylated CUL2) for their action (FIG. 13A). Furthermore, quantitative real-time PCR performed on MDA-MB-231 cells treated with 250 nM of exemplary compound 50 or 46 for 24 hours revealed no significant changes in mRNA for either p38 isoform, demonstrating that these PROTACs do not downregulate p38α and p38δ at the transcriptional level (FIG. 13B). Additionally, MDA-MB-231 cells were pre-treated with 100 μg/mL of cycloheximide (CHX) and chased with 250 nM PROTAC for 30 to 360 minutes, revealing rapid degradation half-lives of exemplary compound 50 on p38α and exemplary compound 46 on p38δ (FIG. 13C). Taken together, these assay results revealed that neither PROTAC resulted in substantial downregulation of the other p38 isoform either before or at the level of de novo synthesis (since CHX stalls protein synthesis), indicating bona fide degradation of only their respective p38 isoform. Lastly, while characterizing the mechanism of action of exemplary compound 50 and exemplary compound 46 their duration of effect upon washout was monitored, as PROTACs have been shown to exhibit a catalytic mechanism of action. MDA-MB-231 cells treated for 24 hours with either exemplary compound 50 or exemplary compound 46 displayed potent, isoform-selective degradation. However, when an additional set of cells treated for 24 hours were rinsed with PBS (to remove excess extracellular compound), dissociated from culture dishes, and re-plated onto new culture plates with fresh medium devoid of any compound (“washout” cells), sustained PROTAC-induced p38 degradation was still observed (FIG. 13D). In particular, exemplary compound 50 maintained p38α degradation efficacy for 72 hours post-washout, while exemplary compound 46 only maintained p38δ degradation efficacy for 24 hours post-washout. These results indicate that these two exemplary compounds degrade their respective p38 isoform in a rapid, sustained, and proteasome-dependent manner.

p38α Degradation Selectivity is Driven by a Favorable PROTAC-Induced Ternary Complex

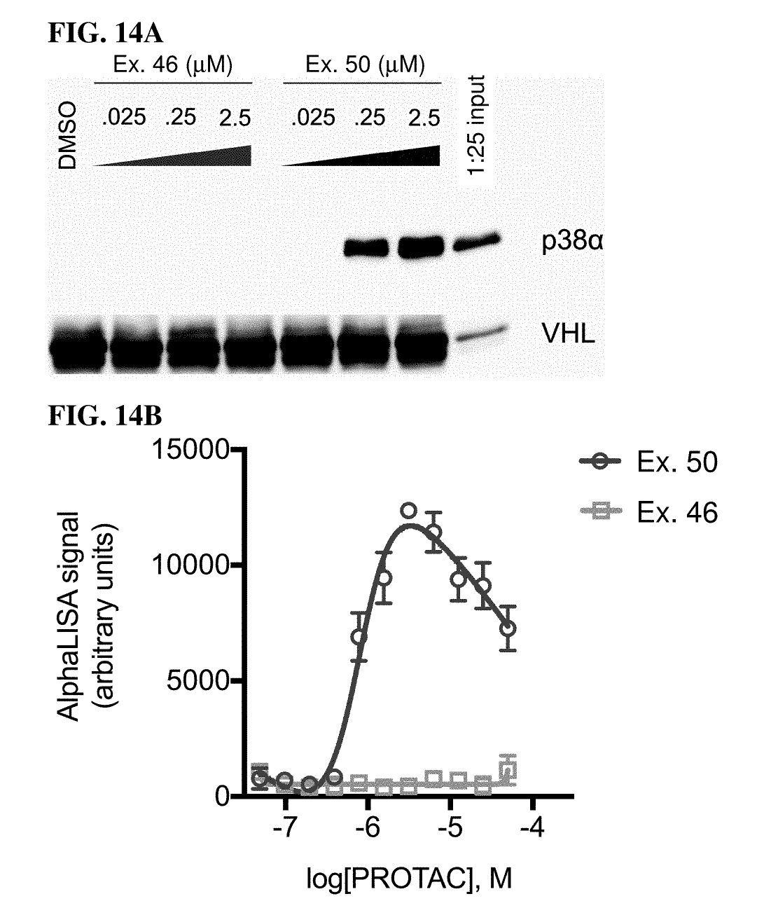

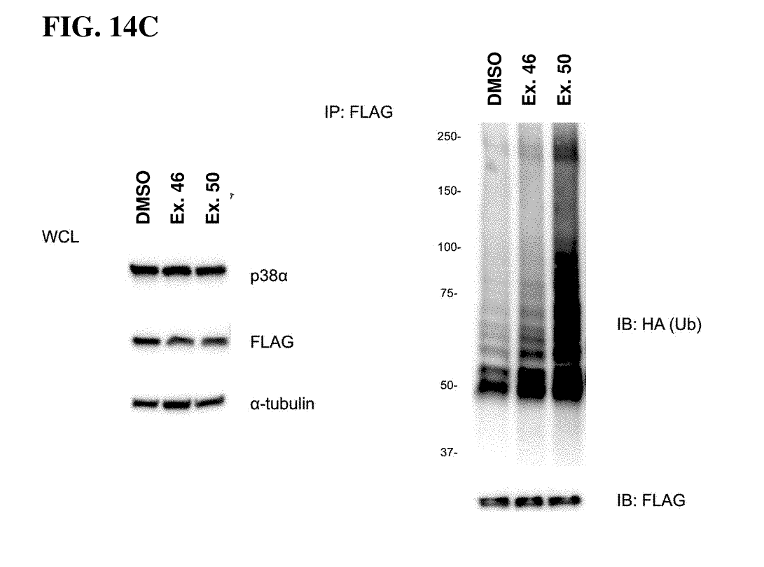

Previous work has demonstrated the profound influence of favorable protein-protein interactions (PPIs)—that occur at the PROTAC-induced interface between a given target protein and recruited E3 ligase—on PROTAC degradation outcomes. In one particular study, it was revealed that the weak binary affinity of a foretinib-based PROTAC for p38α can be overcome by direct and cooperative binding contributions between VHL and p38α to form a higher affinity ternary p38α:PROTAC:VHL complex. Following this logic, to the present disclosure seeks to describe the selectivity of exemplary compound 50 for p38α at the ternary complex level through the use of an in vitro ternary complex pull-down assay. Briefly, glutathione 5-transferase (GST)-tagged VHL/ElonginB/ElonginC (VBC) “bait” was immobilized on glutathione sepharose beads to trap any potential ternary complex that occurs between VHL and substrate protein when incubated in the presence of a PROTAC. Using this GST-VBC “bait”, substantial enrichment of purified p38α was observed only in the presence of exemplary compound 50 and no detectable enrichment at any concentration of exemplary compound 46 tested (FIG. 14A). Similarly, exemplary PROTACs 46 and 50 were evaluated in a proximity-based luminescence assay (AlphaLISA) to detect ternary complex formation. Corroborating the in vitro pull-down experiment, only exemplary compound 50 displays a ternary VHL:PROTAC:p38α complex and no such ternary species is detectable when p38α and VHL are incubated in the presence of increasing concentrations of exemplary compound 46 (FIG. 14B).

Following these observations, it reasoned that only exemplary compound 50 would be able to induce substantial ubiquitination of p38α in a cellular system. Indeed, when HeLa cells co-expressing FLAG-tagged p38α (˜42 kDa) and HA-ubiquitin (HA-Ub, ˜10 kDa) were treated with vehicle (DMSO), 500 nM exemplary compound 46, or 500 nM exemplary compound 50 for 1 hour, only FLAG-immunoprecipitated p38α in the exemplary compound 50-treated samples displayed substantial ubiquitination (HA-Ub smear). Notably, this effect was not seen in the FLAG-immunoprecipitated p38α in the DMSO- and exemplary compound 46-treated samples (FIG. 14C).

Interestingly, DMSO- and exemplary compound 46-treated samples revealed some p38α mono-, di-, and tri-Ub conjugates, however, only exemplary compound 50 displayed considerable amounts of the high molecular weight (HMW) poly-Ub conjugates required for proteasomal degradation. Both the signal intensity and size of these exemplary compound 50-induced poly-Ub chains (up to ˜100 kDa) are indicative of a p38α protein with up to ˜6-7 ubiquitin proteins attached (FIG. 14C). Thus, these exemplary compound 50-induced p38α-Ub conjugates correlate with the substantial p38α:compound 50:VHL ternary complex observed (FIGS. 14A and 14B), in addition to the rapid, sustained, and selective degradation of p38α by exemplary compound 50 and not by exemplary compound 46 (FIGS. 9A, 9B, 9C, 9D, 13A, 13B, 13C, and 13D).

Ternary Complex Affinity Differences Drive p38δ Degradation Selectivity

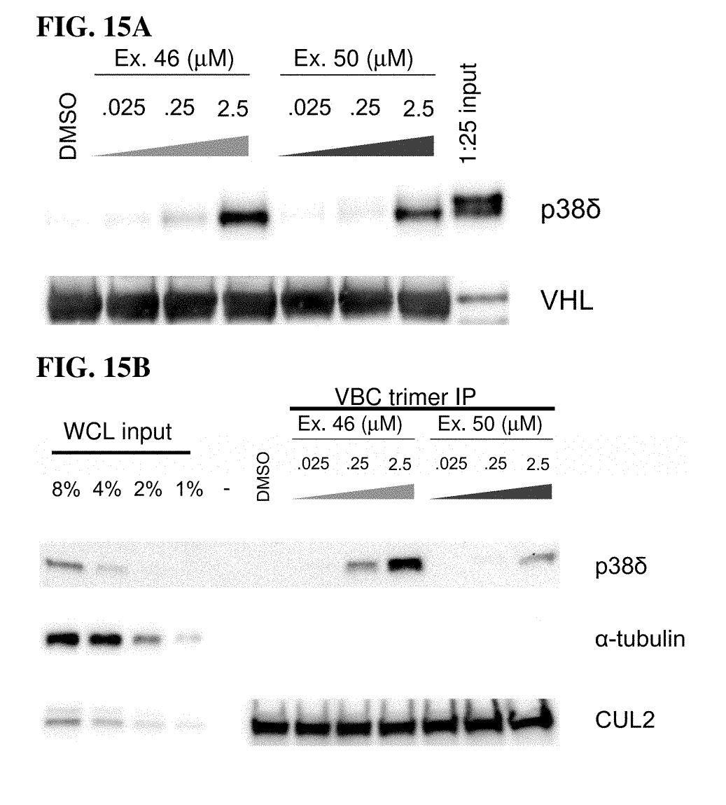

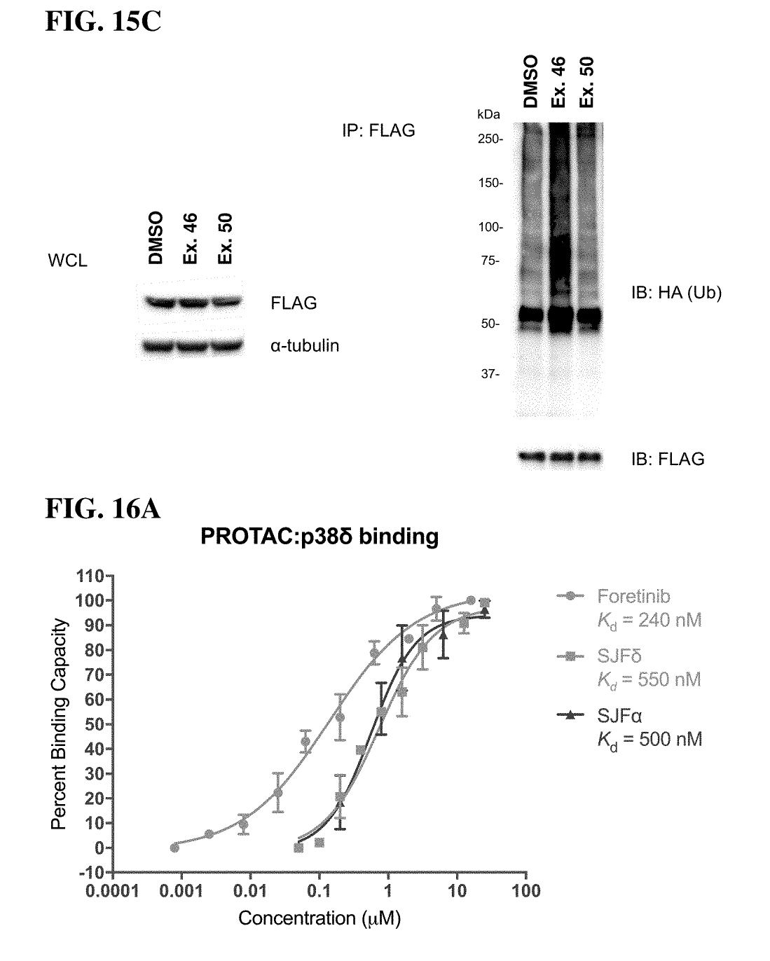

Upon discovering that exemplary compound 50 degradation selectivity for p38α occurs at the level of the ternary complex, a similar level of understanding on exemplary compound 46 selectivity for p38δ was examined. Using the aforementioned in vitro ternary complex assay, it was determined that both exemplary compound 50 and exemplary compound 46 are able to pull-down purified p38δ on GST-VBC-coated beads in a dose-dependent manner, with exemplary compound 46 showing only slightly greater ternary complex pull-down efficiency than exemplary compound 50 at all concentrations tested (FIG. 15A). This curious finding—that non-degraded proteins can engage in PROTAC-induced ternary complexes—was recently reported, but not described with comparison to PROTACs that promote degradation of those same targets. This intriguing possibility with the present system in which two PROTACs can engage with p38δ in a ternary complex in vitro, but result in different cellular outcomes (one that results in target degradation, exemplary compound 46, and one that does not, exemplary compound 50) was examined. Thus, the p38δ:PROTAC:VHL ternary complexes were quantivatively characterized using surface plasmon resonance (SPR) in order to measure binary and ternary complex affinities and assess the relative contributions of assembly kinetics in dictating these affinities.

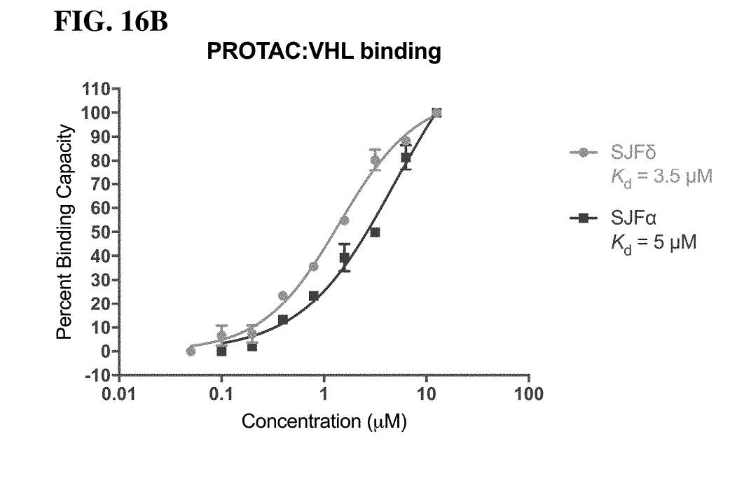

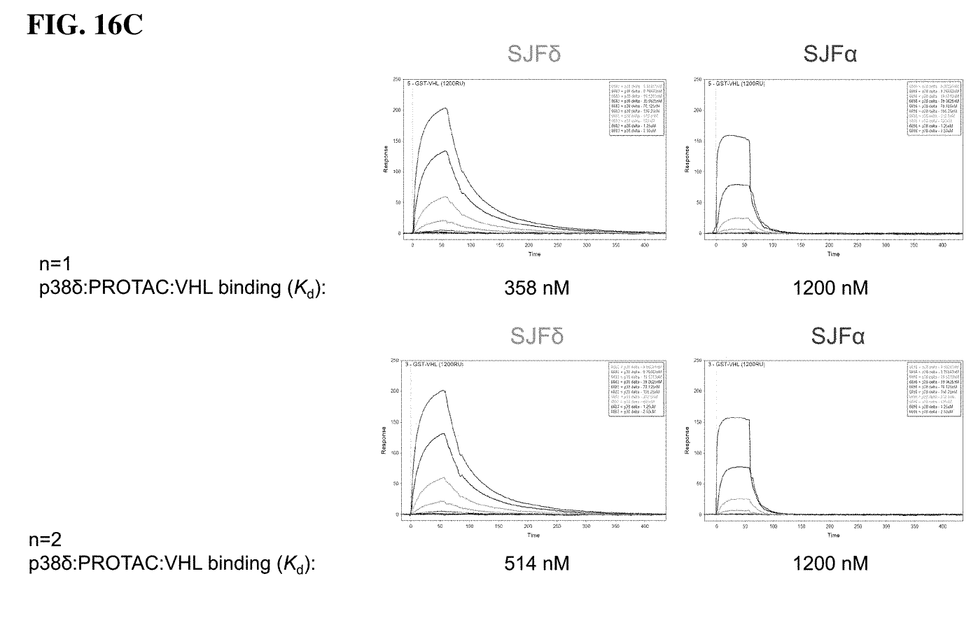

In SPR experiments, parent PTM/warhead foretinib, exemplary compound 50, and exemplary compound 46 were injected onto immobilized p38δ (His-tagged p38δ) in separate channels in order to obtain binary p38δ:compound affinity measurements (FIG. 16A). Foretinib displays an affinity of 240 nM—in agreement with previous measurements—and exemplary compound 50 (K_{d}=500 nM) and exemplary compound 46 (K_{d}=550 nM) show about 2-fold loss of affinity for p38δ compared to the PTM. Using immobilized VHL (GST-VBC), binary binding affinities were obtained for VHL:exemplary compound 50 (K_{d}=5.0 μM) and VHL:exemplary compound 46 (K_{d}=3.5 μM) (FIG. 16B). Next, with the same setup, equimolar p38δ:PROTAC dilutions were successively injected onto immobilized VHL for 60 seconds, prior to monitoring complex dissociation for the remaining 300 seconds. This SPR strategy enabled us to determine effective ternary affinities for a given p38δ:PROTAC:VHL complex and compare the relative affinity change that occurs between VHL:PROTAC when p38δ is present in the mixture. As shown in Table 12, the observed p38δ:compound 46:VHL affinity is 436 nM, which represents an ˜8-fold leftward shift—nearly an order of magnitude improvement in affinity—from the VHL:PROTAC binary affinity. This “enhanced” composite affinity is likely due to the additional PPIs between VHL and p38δ when in the presence of exemplary compound 46. Exemplary compound 50, however, displayed a p38δ:PROTAC:VHL affinity of 1.2 μM, which represents a small improvement over the cognate binary affinity, but is nevertheless ˜3-fold weaker than the exemplary compound 46-induced complex. From a kinetics perspective, both p38δ:exemplary compound 46 and p38δ:exemplary compound 50 displayed rapid kₒₙ kinetics of 3.96×10⁴ M⁻¹ s⁻¹ and 7.01×10⁴ M⁻¹ s⁻¹, respectively, and both complexes displayed slow dissociation rates. However, the exemplary compound 46 complex (k_{off}=0.0175 s⁻¹) showed increased half-life (t_{1/2}=38 s) and residence time (330 s), compared with the exemplary compound 50 complex (k_{off}=0.0175 s⁻¹, t_{1/2}=8 s, residence time=80 s) (Table 12 and FIG. 16C). These results indicate that p38δ:PROTAC:VHL is the more favorable, highly-populated ternary complex of the two, which correlates with the degradation outcomes seen previously (FIG. 9C).

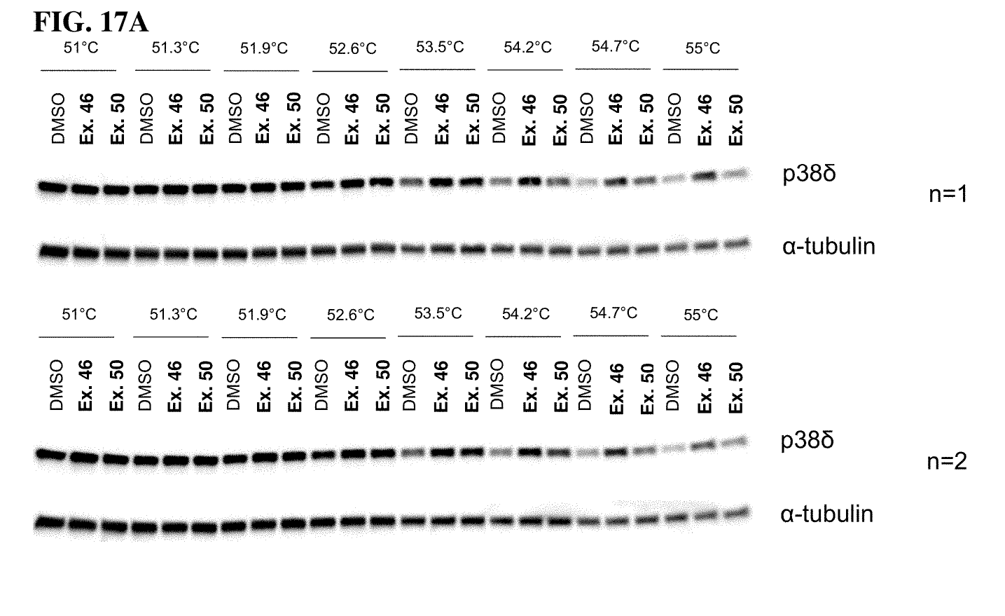

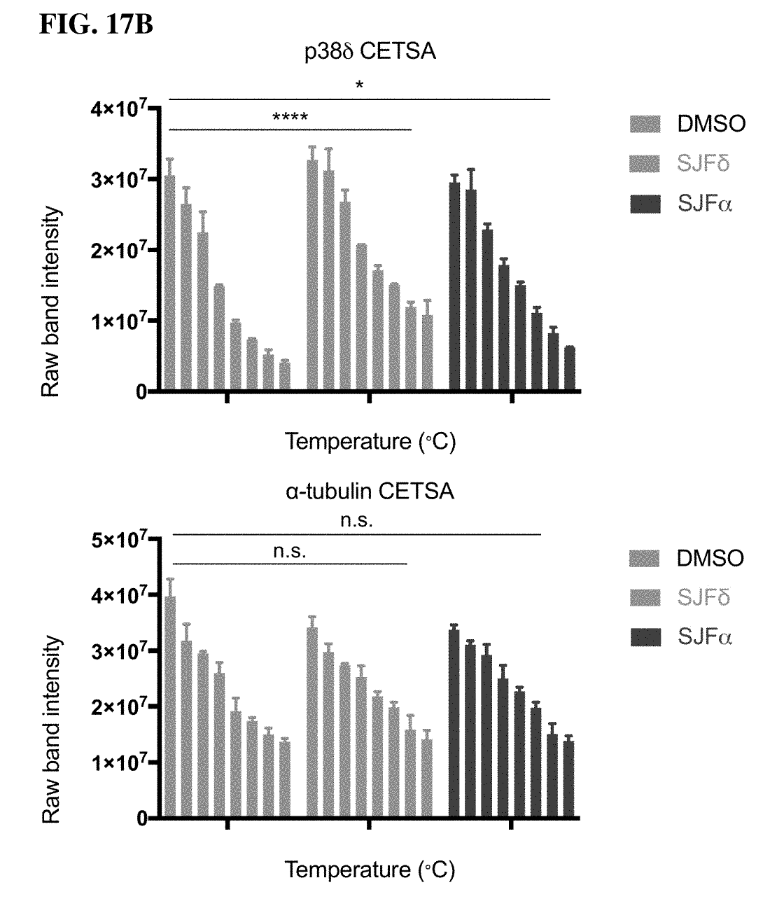

To further assess these findings, a more biologically-relevant context for measuring ternary complex formation was used. Two strategies that utilize MDA-MB-231 whole cell lysate as a source for both endogenous p38δ and VHL, in addition to any associated proteins, were utilized. In the first approach, a modified ternary complex pull-down assay in which whole cell lysate from MDA-MB-231 cells was incubated with vehicle (DMSO) or varying concentrations of either exemplary compound 50 or exemplary compound 46 in the presence of excess immobilized VHL (GST-VBC) bait was utilized. Exemplary compound 46 was found to strongly enrich for endogenous p38δ in a dose-dependent manner and that this stable ternary complex was greatly reduced in the exemplary compound 50-incubated pull-down samples (FIG. 15B). In the second strategy, a cellular thermal shift assay (CETSA) which recently showed that PPIs in cyclin-containing protein complexes can enhance a substrate's thermal stability when subjected to a range of denaturation (“melting”) temperatures was utilized. Thus, whether PROTAC-induced PPIs could be monitored in a complex cellular environment using CETSA was examined. Previously, it was that free (unbound) endogenous p38δ in MDA-MB-231 cells displays half-maximal melting at 53° C. (data not shown) and accordingly the present experiments center around this temperature (51-55° C.). Upon incubation with exemplary compound 50, a small, but statistically-significant (p=0.0121) thermal shift in the p38δ melting profile was observed, indicating cellular target engagement between p38δ and exemplary compound 50 (FIGS. 17A and 17B). However, the thermal stability shifts in p38δ became more pronounced at each melting temperature tested when cells were incubated rather with exemplary compound 46 and showed high statistical-significance (p<0.0001) when compared to DMSO. Notably, thermal stability shifts were not seen when comparing exemplary compound 50 or exemplary compound 46 with DMSO on the negative control protein (α-tubulin) indicating bona fide PROTAC-induced p38δ thermal stabilization. It was reasoned that these exemplary compound 46-induced and exemplary compound 50-induced thermal stability shift differences are not likely due to differences in cellular uptake nor binary affinity, as these CETSA assays were performed with MDA-MB-231 cell lysate and exemplary compound 50 possesses slightly greater binary affinity for p38δ than does exemplary compound 46 (Table 12). Instead, it is believe that the increased exemplary compound 46-induced p38δ thermal stability is due to its increased ternary association with VHL (FIG. 15B, Table 12, FIG. 16C), when compared to exemplary compound 50. Moreover, the differences seen between these two assays and FIG. 15A demonstrate how a more rigorous cellular assessment of ternary complex formation might be required, especially for ternary complexes with weak affinity (p38δ:exemplary compound 50:VHL, see Discussion below). Thus, much like a previous study, this p38δ:exemplary compound 46:VHL “cellular ternary complex” strongly correlates with the selective p38δ degradation observe with exemplary compound 46 in MDA-MB-231 cells (FIG. 9C), and it is now posited that the mere capacity to form a ternary complex is not sufficient for PROTAC-induced substrate degradation.

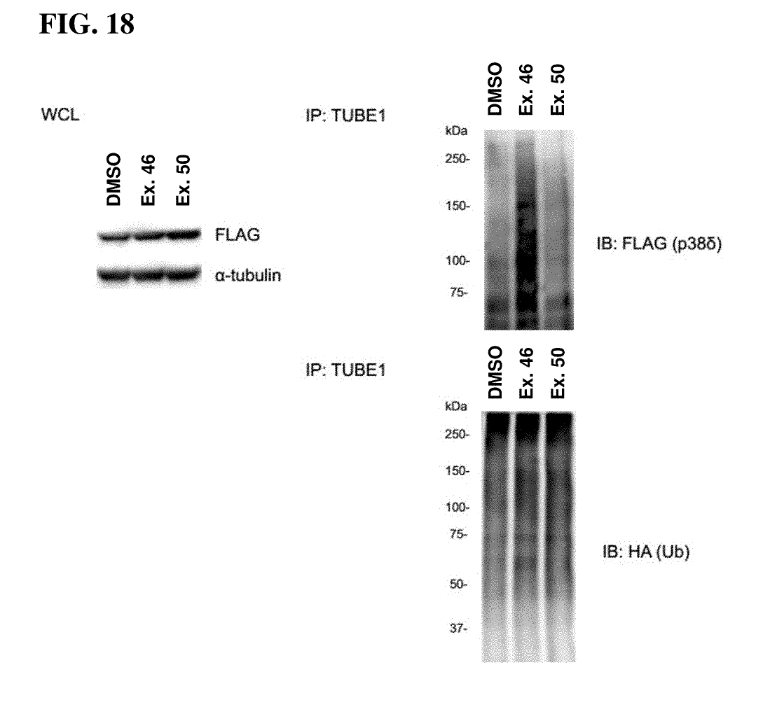

Based on these exemplary compound 50-induced and exemplary compound 46-induced ternary complex affinity differences, both in vitro and in cells, whether they were predictive of PROTAC-induced p38δ cellular ubiquitination was examined. When HeLa cells co-expressing FLAG-p38δ (˜43 kDa) and HA-Ub were treated with vehicle (DMSO) or saturating concentrations of either exemplary compound 50 (1 μM) or exemplary compound 46 (1 μM), only the exemplary compound 46-treated cells displayed levels of ubiquitination of FLAG-immunoprecipitated p38δ greater than control cells (FIG. 15C). Substantial p38δ-Ub conjugates can be seen in the 75-100 kDa region where tetra-ubiquitinated p38δ would be expected to migrate but are far less abundant in the DMSO- and exemplary compound 50-treated samples. Similar immunoprecipitation experiments performed with agarose beads conjugated to the tetra-ubiquitin-capturing tandem ubiquitin binding entities (TUBEs) revealed that only exemplary compound 46-treated cells display increased TUBE1-reactivity (poly-Ub p38δ, FIG. 18). Taken together, these results indicate that exemplary compound 50 and exemplary compound 46 ternary complex affinity differences, in vitro and in cells, result in profoundly different cellular ubiquitination profiles (FIGS. 15C and 18) that may explain the p38δ degradation selectivity of exemplary compound 46 (FIG. 9C).

### DISCUSSION

The present example developed isoform-selective p38 MAPK-targeting PROTACs using one PTM/warhead and one E3 ligase. This work was motivated by previous studies that showed how the use of different targeting ligands for a common protein substrate, as well as the choice of which E3 ubiquitin ligase is recruited (CRBN or VHL), can result in drastically different degradation profiles for the substrate. The present example asked whether it was possible to selectively target one isoform of a protein family over the others and if one could simply “switch” this degradation selectivity through varied linker design and differential orientation of a single E3 ligase. Starting with a foretinib PTM with pan-activity towards the p38 MAPK family, two PROTAC series (“amide” and “phenyl”) were developed that differentially recruit VHL and, in doing so, were able to achieve selective p38α/p38δ degradation profiles with exemplary compounds 50 and 46.

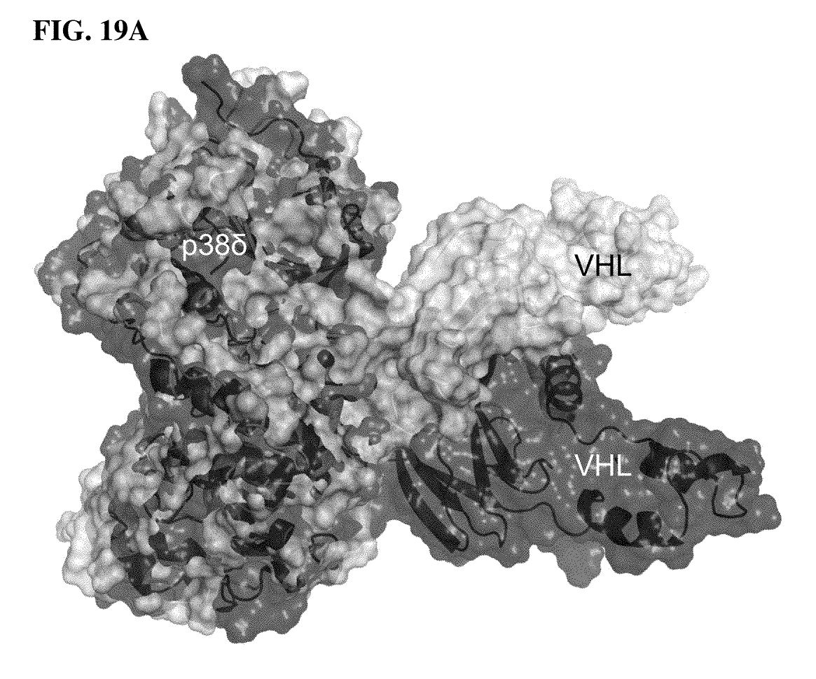

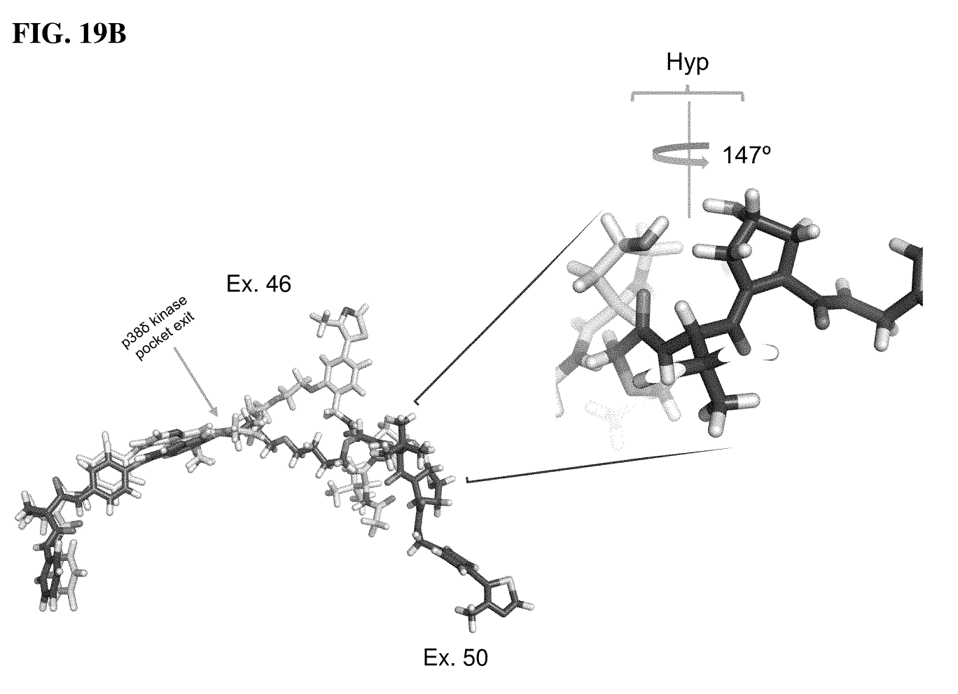

Through the present work, how single atom alterations to PROTAC linkers could change chemical probes with dual p38α/p38δ degradation activity (exemplary compounds 49 and 48) into compounds with enhanced selectivity for the p38α isoform (exemplary compounds 1 and 50) (FIGS. 11A and 11B, Table 13) were identified. These amide series PROTACs were based on the oft-utilized “left-hand side” VHL-recruiting moiety and displayed a requirement for “longer” linker lengths in order to achieve this p38α selectivity (exemplary compound 50=13-atom linker). Interestingly, however, only exemplary compound 46 of the phenyl series PROTACs—which employ a “right-hand side” VHL ligand linkage—demonstrated appreciable activity towards either p38 isoform, and this PROTAC series displayed a preference for “shorter” linker lengths to ultimately achieve p38δ efficacy and selectivity (exemplary compound 46=10-atom linker). While it is possible that VHL might possess an inherent “preference” for interfacing with a given p38 MAPK isoform, it is believed that such differences are insignificant due to (i) high structural conservation between the p38 isoforms (FIG. 12C), and (ii) the fact that the “shorter” amide series PROTACs (10-atom and 11-atom) can show relatively equipotent degradation of p38α and p38δ. Instead, it is believed that these atomic linker length “preferences” are biased by the VHL-recruiting ligands themselves and selectivity is further enhanced by linker length exploration. Based on the linkage vectors of the two VHL ligands (FIGS. 19A and 19B), it is believed that the phenyl attachment provides a more “direct” VHL recruitment that does not require the PROTAC linker region to bend back on itself, as seen in the only PROTAC crystal structure to date (PDB: 5T35) which is based on an amide-linked VHL-recruiting ligand. This “kinked” linker conformation was also seen in a previous study, in which a molecular dynamics (MD) simulation of the p38α:PROTAC:VHL ternary complex revealed a residue on p38α (Ala40) that favorably interacted with the PROTAC linker region. While these kinked linkers seen in the amide-linked VHL PROTACs appear to provide additional surface for favorable contact in between the substrate:ligase interface, subtle changes to these contacts—either through slight modifications in linker length or composition—appear to result in drastic enhancements/reductions in potency and selectivity (as seen in FIGS. 11A and 11B, and Table 13). In fact, the lack of degradation seen with phenyl-linked VHL PROTACs in a previous report might be a consequence of underexplored linker space and the fact that amide-linked and phenyl-linked VHL PROTACs can have different linker length requirements per given substrate. Thus, the work done previously and in the present application demonstrate the notion that PROTAC linkers represent a delicate balance between affinity contributions and steric effects.

In order to model these linker length and VHL recruitment questions, MD simulations have been performed on the p38δ:exemplary compound 46:VHL and p38δ:exemplary compound 50:VHL ternary complexes, in which previously-published p38δ and VHL structures were docked and the two ternary structures were allowed to separately relax into a low energy conformation. These short (100-ns) MD simulations showed that exemplary compound 50 and exemplary compound 46 result in a recruited VHL that docks onto p38δ in two entirely different conformations (FIG. 19A). According to these models, the pentameric E3 ligase complex (VHL/EloB/EloC/CUL2/Rbx1) could access different faces of p38δ as a consequence of the two contorted VHL conformations seen in the exemplary compound 50-recruited and exemplary compound 46-recruited ternary complexes. Moreover, upon inspecting the conformation of each PROTAC in these models, it was found that the 10-atom phenyl-linked exemplary compound 46 directly interacts with VHL outside the p38δ kinase pocket, whereas the 13-atom amide-linked exemplary compound 50 takes a “turn” out of the p38δ kinase pocket, kinking along the way, to recruit a VHL that results in a twisted conformation, relative to the exemplary compound 46-recruited p38δ (FIG. 18B). These comparative models highlight how linker length and orientation of the recruited E3 ligase can result in different ternary interfaces (with inherent ternary affinity differences) that can achieve selective degradation with one PROTAC over another. Thus, the use of the phenyl-linked VHL ligand in addition to the amide-linked VHL ligand—with further linker length and composition exploration—might offer PROTACs with access to harder-to-degrade protein targets through differential recruitment of VHL.

In an effort to understand p38 MAPK isoform selectivity, the ability for individual PROTACs to form a stable ternary complex with either p38α or p38δ was investigated, as previous work showed that this “step” was highly predictive for PROTAC-mediated degradation of a given substrate. In that work, however, two exceptions to this principle—the kinases c-Abl and Arg—also showed profound ternary complex enrichment with a PROTAC that did not degrade these substrates. In the present example, stable ternary complex formation between p38α:PROTAC:VHL was identified as the driving force behind the selectivity for exemplary compound 50-promoted (and against exemplary compound 46-promoted) p38α degradation (FIGS. 14A, 14B, and 14C). Alternatively, the mere presence of a ternary complex between p38δ:PROTAC:VHL was identified as not being predictive for subsequent degradation, but, rather, a more nuanced understanding was required to appreciate the exemplary compound 46 degradation selectivity for p38δ (FIGS. 15A, 15B, and 15C). The findings of the present example point to differences in ternary complex affinity (and the associated metrics of off-rates, half-lives, and residence times) as the driving force behind exemplary compound 46-selective degradation of p38δ. Despite the ability for a p38δ:exemplary compound 50:VHL complex to form, the affinity for such a complex is >1 μM with an off-rate that is ˜5-fold quicker and a residence time that is ˜4-fold shorter than the p38δ:exemplary compound 46:VHL complex (Table 12).

While the role that residence time plays in proper PROTAC function is yet to be uncovered, it was recognized that this consideration might be entirely empirical per substrate:ligase pair, as the accessibility of substrate lysines, processivity of a given recruited E3 ligase complex, proteasomal recognition of ubiquitinated lysines, ubiquitin chain linkage patterns, identity of PROTAC-recruited E2 enzymes, and the role that different E2 enzymes play (initiation vs. extension) is yet to be understood. Despite these nuanced considerations, it is believe that ternary complex affinity is the main driving force behind exemplary compound 46-mediated selective degradation of p38δ, as these in vitro ternary complex affinity measurements correlate in cells where exemplary compound 50 engagement with p38δ is reduced compared to exemplary compound 46 (FIGS. 15B, 17A and 17B), resulting in a lack of significant cellular ubiquitination of p38δ with exemplary compound 50 (FIGS. 15C, 18A, and 18B). Moreover, it is not believed that lysine accessibility differences between the p38δ:exemplary compound 50:VHL and the p38δ:exemplary compound 46:VHL complexes likely explain selectivity (despite differences in the modeled p38δ:VHL architecture), since p38δ not only possesses twice as many lysines as p38α (32 vs. 16), but these lysines are distributed throughout its surface and 11 of these lysines are homologous between p38δ and p38α (FIG. 12B). Furthermore, the fact that minimal degradation of p38δ is seen with exemplary compound 50 even at 5 μM (above the measured ternary complex K_{d}) hints at the fact that the p38δ:exemplary compound 50:VHL complex is lowly-populated and inefficient. Lastly, it has not been ruled out that this could also be due to competition of exemplary compound 50:VHL with natural binding partners of p383, of which few are known. While no studies have shown competition between PROTAC:VHL and associated proteins of a substrate, it is believed that this is entirely possible. Based on the modeled docking interface of p38δ:PROTAC:VHL (FIGS. 19A and 19B), it appears that VHL binds to a highly-conserved docking groove present in every MAPK family. As p38δ is an understudied kinase, few studies have biochemically characterized p38δ docking groove interactions. However, this common docking groove—to which upstream kinases, inactivating phosphatases, and downstream substrates bind—displays μM affinities for these interactions with p38α. Thus, it is possible that the relative ternary complex affinity differences that occur between p38δ:exemplary compound 50:VHL and p38δ:exemplary compound 46:VHL in vitro become magnified in a cellular milieu of docking groove competition. Future studies will investigate whether or not specific target protein binding partners can contribute to unsuccessful PROTAC-induced degradation and if this hypothesis could explain why certain targets are ‘harder to degrade’ using the PROTAC approach.

Overall, two PROTACs that target individual isoforms of the p38 MAPK family were identified. Selective and potent degradation of p38α (<10 nM) and p38δ (<100 nM) was achieved. There are no FDA-approved therapeutics that currently exist for p38α and p38δ despite the vast disease relevance of the former and due to the “undruggable” status of the latter. The present disclosure highlights the general applicability of the PROTAC technology for rapidly achieving selective degradation of protein targets with nM potency using a single E3 ligase and PTM. Furthermore, through exploration of linker length and E3 ligase recruitment geometry, the necessity of the ternary complex for protein degradation was underscored. However, the ternary complex is not sufficient for protein degradation. The present disclosure describes, for the first time, biophysical measurements of PROTAC-induced ternary complex association and dissociation kinetics and comparatively describes the cellular effects of the differences that lie therein. Further work will need to be performed to understand if the lessons learned here extend beyond the p38 MAPK family and if the approach can be used to target “undruggable” proteins with few available ligands.

Materials and Methods

Cell culture. MDA-MB-231 and HeLa cells were obtained from the American Type Culture Collection (ATCC), cultured in RPMI-1640 (1×) and DMEM (1×) medium, respectively, containing 10% fetal bovine serum (FBS) and 1% penicillin-streptomycin and grown in a humidified incubator at 37° C. and 5% CO₂.

Immunoblotting.

Lysates from MDA-MB-231 cells were washed once with ice cold PBS (1×), followed by lysis in buffer containing 25 mM Tris [pH 7.5], 0.25% sodium deoxycholate, 1% Triton X-100, supplemented with 1× protease inhibitor cocktail (Roche) and phosphatase inhibitors (10 mM NaF, 1 mM Na₃VO₄, and 20 mM β-glycerophosphate), unless otherwise noted. Lysates were spun at 14,000×g for 10 minutes at 4° C. and supernatant was evaluated for protein content using a Pierce BCA Protein Assay (Thermo Fisher Scientific). 25 to 50 μg of protein was loaded onto 10% SDS-PAGE gels or 4-20% Criterion TGX precast gradient gels (Bio-Rad), transferred to nitrocellulose membranes, and probed with the specified antibodies overnight at 4° C. in 1×TBS-Tween (Tris buffered saline plus 0.02% Tween 20) containing 5% non-fat milk. Immunoblots were visualized using a Bio-Rad ChemiDoc imaging instrument and subsequently processed and quantified using the accompanying Bio-Rad ImageLab software.

Rabbit antibodies purchased from Cell Signaling Technologies (CST): p38α (#9218), p38δ (#2308), p38β (#2339), p38γ (#2307), ERK2 (#9108), JNK2 (#9258), and VHL (#68547). Additional CST antibodies were also purchased: mouse HA-tag (#2367), mouse JNK1 (#3708), and FLAG (DYKDDDDK) Tag Sepharose bead conjugate (#70569). Mouse antibodies purchased from Sigma-Aldrich: alpha-tubulin (#T9026) and FLAG M2 (#F1804). Rabbit Cullin 2 (CUL2) was purchased from ThermoFisher Scientific (#700179) and rabbit ERK1 (C-16) from Santa Cruz Biotechnology (#sc-93).

Constructs, protein expression and purification.

A plasmid containing an N-terminal His₆ tag and encodes a region spanning amino acids 2-360 of the human p38α kinase (NCBI Reference Sequence: NM_139012). BL21-CodonPlus(DE3)-RIPL E. coli cells (Agilent Technologies) were transformed with pMCSG7-His₆-p38α and were selected in LB medium containing carbenicillin (100 μg mL⁻¹), chloramphenicol (15 μg mL⁻¹), and spectinomycin (50 μg mL⁻¹) at 37° C. until OD₆₀₀=0.6-0.8. At this point, cells were induced with 1 mM isopropyl β-D-1-thiogalactopyrano-side (IPTG) and grown at 25° C. for 14-16 hours. Cell pellets were collected by centrifugation (5,000 rpm, 10 min, 4° C.) and homogenized in lysis buffer (10 mM Tris pH 8.3, 500 mM NaCl, 5 mM β-mercaptoethanol, 10 mM imidazole, and 10% glycerol) containing a 1× protease inhibitor cocktail tablet (Roche). The homogenized cells were subsequently passed through a microfluidizer three times at 15 k PSI and lysate was clarified by centrifugation (16,000 rpm, 45 minutes, 4° C.). The resultant supernatant was then applied to Ni-NTA agarose beads (QIAGEN) with gentle rotation for 1 hour at 4° C., washed once with lysis buffer with 10 mM imidazole (pH 8.3), twice with lysis buffer containing 20 mM imidazole (pH 8.3), and eluted off of the nickel resin in lysis buffer containing 50 mM imidazole (pH 8.3). Eluted protein was assessed for identity and purity via coomassie staining of sample run on an SDS-PAGE gel and pure elutions were pooled, concentrated, and diluted in ion-exchange buffer A (10 mM Tris pH 8.3, 5 mM β-mercaptoethanol) until the salt concentration was 50 mM, before loading onto a Mono Q 5/50 GL column (GE Life Sciences). The protein was subjected to a step-wise wash protocol, followed by a linear gradient from 200-500 mM NaCl using ion-exchange buffer B (10 mM Tris 8.3, 1 M NaCl, 5 mM β-mercaptoethanol). Fractions were then assessed for purity via coomassie, pooled, concentrated, and run on a HiLoad 16/600 Superdex-200 column (GE Healthcare Life Sciences) using size-exclusion buffer (10 mM Tris pH 8.3, 150 mM NaCl, 5 mM β-mercaptoethanol). Pure fractions of p38α were pooled, concentrated to ˜5 mg mL⁻¹, aliquoted, and flash-frozen before storing at −80° C.

Wild-type human p38δ kinase (NCBI Reference Sequence: NM_002754.4) encoding a region spanning amino acids 1-365 was PCR-amplified from a pcDNA3.3-p38delta-MAPK (MAPK13) template that we received as a gift from Dr. Romeo Ricci (IGBMC), described previously in Sumara G, et al. (2009) Regulation of PKD by the MAPK p38delta in insulin secretion and glucose homeostasis. Cell 136(2):235-248. This p38δ region was cloned into a pET28a vector containing an N-terminal His₆ tag using NheI and BamHI restriction sites. As before, BL21-CodonPlus(DE3)-RIPL E. coli cells (Agilent Technologies) were transformed with pET28a-His₆-p38delta and were selected in LB medium containing kanamycin (50 μg mL¹), chloramphenicol (15 μg mL⁻¹), and spectinomycin (50 μg mL⁻¹) at 37° C. until OD₆₀₀=0.8. Purification of His₆-p38δ was performed identical to His₆-p38α above, except Ni-NTA agarose beads were washed twice with lysis buffer with 20 mM imidazole (pH 8.3), once with lysis buffer containing 50 mM imidazole (pH 8.3), and eluted off of the nickel-conjugated resin in lysis buffer containing 150 mM imidazole (pH 8.3). Pure fractions of p38δ were pooled, concentrated to ˜3 mg mL⁻¹, aliquoted, and flash-frozen before storing at −80° C.

For the expression of GST-tagged VHL:Elongin B:Elongin C (herein referred to as GST-VHL), wild-type human VHL, Elongin B, and Elongin C were co-expressed in E. coli. BL21(DE3) cells were co-transformed with pBB75-Elongin C and pGEX4T-2-VHL-rbs-Elongin B and selected in LB medium containing carbenicillin (100 μg mL⁻¹) and kanamycin (25 μg mL⁻¹) at 37° C. until OD₆₀₀=0.8, at which point the culture was chilled to 16° C. and induced with 0.4 mM IPTG for 16 hours. Cells were homogenized and lysed, as described above, except the lysis buffer was composed of 30 mM Tris [pH 8.0], 200 mM NaCl, 5% glycerol, 5 mM DTT containing a 1× protease inhibitor cocktail tablet (Roche). Clarified cell lysate was applied to Glutathione Sepharose 4B beads (GE Life Science) and gently rotated for 2 hours at 4° C. Beads were washed with four column volumes of lysis buffer, followed by four column volumes of elution buffer (50 mM Tris pH 8.0, 200 mM NaCl, 10 mM Glutathione). Eluted protein was assessed for identity and purity via coomassie staining of sample run on an SDS-PAGE gel and pure elutions were pooled, concentrated, and diluted in ion-exchange buffer A (30 mM Tris pH 8.0, 5% glycerol, 1 mM DTT) until the salt concentration was 50 mM, before loading onto a Mono Q 5/50 GL column (GE Life Sciences). The protein was subjected to a linear gradient of NaCl (0-500 mM NaCl) using ion-exchange buffer B (30 mM Tris 8.0, 1 M NaCl, 5% glycerol, 1 mM DTT). Fractions were then assessed for purity via coomassie, pooled, concentrated, and run on a Superdex-200 column (GE Life Sciences) using size-exclusion buffer (30 mM Tris pH 8.0, 100 mM NaCl, 10% glycerol, 1 mM DTT). Pure fractions of GST-VHL were pooled, concentrated to ˜5 mg mL⁻¹, aliquoted, and flash-frozen before storing at −80° C.

Ternary Complex Pulldown.

Glutathione Sepharose 4B (Glutathione-conjugated beads in a slurry containing 20% ethanol) were washed twice with 1× wash buffer (50 mM HEPES pH 7.5, 150 mM NaCl, 1 mM DTT, 0.01% NP40, 5 mM MgCl2, 10% Glycerol) and then blocked for one hour at room temperature with 10% Bovine Serum Albumin (BSA) in wash buffer. The beads were then washed again thrice with wash buffer and then purified GST-VBC (stable form of VHL complexed with EloB/EloC, described above) was immobilized for one hour at 4° C. at 3.6 pmole per μL of beads. The beads were then washed thrice with wash buffer, resuspended, divided into two equal volumes, and p38α or p38δ protein was added. The bead:p38 mixture was then aliquoted to separate tubes and PROTAC was added at the indicated concentration (PROTACs were intermediately diluted in 10% DMSO and 0.25% CHAPS). This mixture was incubated at 4° C. for two hours. The beads were washed thrice with 10 column volumes of wash buffer and then eluted with SDS loading buffer at 75° C. for 10 minutes.

For experiments in which the input substrate is a whole cell lysate (FIG. 15B), the sample was prepared as follows. Five 150 mm dishes of confluent MDA-MB-231 cells were washed with 1×PBS, pH 7.4, and then dissociated using enzyme-free PBS-based cell dissociation buffer (ThermoFisher Scientific) for 10 minutes at 37° C. Cells were then pelleted, resuspended in wash buffer (same as above, but supplemented with 1× protease inhibitor cocktail (Roche) and 5 μM epoxomicin), and then lysed by sonication (Branson sonicator microtip, power=6, 50% duty cycle for 3 cycles of 30 seconds on and 1 minute off at 4° C.). The lysate was cleared by centrifugation and then added to the GST-VBC-conjugated beads as an input substrate, as above.

AlphaLISA Ternary Complex Assay.

Assays were performed at room temperature and plates sealed with transparent film between addition of reagents to prevent well contamination. All reagents were diluted in 50 mM HEPES pH 7.5, 50 mM NaCl, 69 μM Brij-35, and 0.1 mg mL⁻¹ BSA. Recombinant GST-VBC was mixed with His₆-p38α and PROTAC (diluted one-in-three from 6× stock) to a final volume of 15 μL per well in a OptiPlate-384 well microplate (PerkinElmer) and incubated for 30 minutes. VBC and p38α were kept at a constant final concentration of 150 nM. 7.5 μL of Anti-6×His AlphaLISA Acceptor beads (PerkinElmer) were added to each well and plates were incubated for 15 minutes in the dark. 7.5 μL of Alpha Glutathione Donor beads (PerkinElmer) were added to each well and plates were incubated for 45 minutes in the dark. Plates were read on a Synergy 2 microplate reader using the Gen5 imager software (BioTek Instruments) with an excitation wavelength of 680 nm and emission wavelength of 615 nm. Intensity values were plotted in Graphpad Prism with PROTAC concentration values represented on a log 10 scale.

Cellular Thermal Shift Assay (CETSA).

CETSA protocol was adapted from (Mateus A, Maatta T A, & Savitski M M (2016) Thermal proteome profiling: unbiased assessment of protein state through heat-induced stability changes. Proteome Sci 15:13). 3×10⁷ MDA-MB-231 cells (1×10⁷ cells per condition) were collected and resuspended in ice-cold 1×PBS supplemented with 1× protease inhibitor cocktail (Roche) and lysed by three cycles of liquid nitrogen snap freeze, followed by 50% thaw in a room temperature water bath and an additional 50% thaw at 4° C. After each freeze-thaw cycle, lysate was vortexed briefly to ensure homogenous thawing. Lysate was then cleared for 20 minutes by centrifugation (14,000×g at 4° C.) and the soluble fraction was divided into three equal aliquots. The aliquots were treated with vehicle (2.5% DMSO), 100 μM exemplary compound 50, or 100 μM exemplary compound 46 and incubated at room temperature for 30 minutes with gentle rotation. Each aliquot was then divided (50 μL) into eight PCR tubes and individually heated at the indicated temperature for 3 minutes followed by cooling at room temperature for 3 minutes. After cooling, samples were spun down for 20 minutes (14,000×g at 4° C.) and the supernatant (soluble fraction) was analyzed by SDS-PAGE and immunoblotted for p38δ and alpha-tubulin (negative control).

Surface Plasmon Resonance (SPR).

SPR experiments were conducted on a Biacore 3000 instrument (GE Healthcare) at room temperature. GST antibody was immobilized through direct amination onto a carboxymethylated dextran surface (CM5) and GST-VBC was captured on the CM5 surface. This prepared surface was equilibrated over three hours in running buffer (10 mM HEPES buffer pH 7.4, 150 mM NaCl, 0.4 mg mL⁻¹ BSA, 0.005% P20, 2% DMSO). All compounds were prepared in 100% DMSO stock plates with a top concentration of 500 μM in a 3× serial dilution. Exemplary compounds were transferred from the stock plate to the assay plate and diluted into running buffer without DMSO. Equimolar concentration of target protein was added to corresponding compound wells. All exemplary compounds were run as a twelve point-concentration series with a final assay top concentration of 50 μM to measure p38δ:PROTAC and VHL:PROTAC binding and top concentration of 5 μM to measure p38δ:PROTAC:VHL binding. All exemplary compounds were injected for 60 seconds and allowed to dissociate for 300 seconds. Data analysis was performed in Scrubber 2 (BioLogic Software). Blanks from reference flow cell containing immobilized GST-VBC were subtracted to correct for noise and data was solvent-corrected against a standard DMSO curve. All reported K_{d} values represent an average of at least n=2 and were obtained by fitting to a minimum of five concentrations using a 1:1 fitting algorithm.

Ubiquitination Assays.

HeLa cells (2×10⁶) were seeded into 10 cm dishes and allowed to adhere overnight. On the following day, cells were transfected with 3 μg pcDNA5-FLAG-p38α or pcDNA5-FLAG-p38δ and 1 μg pRK5-HA-Ubiquitin-WT (Addgene plasmid #17608) in Opti-MEM media using Lipofectamine 2000 (Invitrogen). After 6 hours, Opti-MEM media was replaced with DMEM (1×) and cells were grown for an additional 24 hours. After this time, cells were treated with indicated concentrations of either vehicle (DMSO) or PROTAC for the indicated amount of time at 37° C. Cells were then placed on ice, rinsed twice with ice-cold 1×PBS and lysed in 500 μL modified 1×RIPA buffer (25 mM Tris-HCl pH 7.6, 150 mM NaCl, 1% NP-40, 1% sodium deoxycholate, 0.1% SDS) containing 5 mM 1,10-phenanthroline monohydrate, 10 mM N-ethylmaleimide, 20 μM PR-619, and 1× protease inhibitor cocktail (Roche). Lysates were spun down at 14,000×g at 4° C. for 20 minutes and protein content was measured by Pierce BCA Protein Assay (ThermoFisher Scientific). Protein lysate was normalized and 1 mg of lysate was aliquoted onto 20 μL (bed volume) of DYKDDDDK-sepharose beads (CST: #70659). FLAG-containing proteins were immunoprecipitated from HeLa lysates overnight at 4° C. with gentle rotation, after which samples were spun down at 3,000×g at 4° C. for 2 minutes and the beads were washed once with ice-cold lysis buffer and thrice with ice-cold 1×TBS-T (137 mM NaCl, 2.7 mM KCl, 19 mM Tris-HCl pH 7.5, 0.02% Tween-20). Beads were resuspended in 1×LDS sample buffer containing 5% BME. Immunoprecipitated protein was eluted off of the beads by heating at 95° C. for 5 minutes and the supernatant was run on an SDS-PAGE gel and evaluated for the presence of immunoprecipitated FLAG-tagged proteins (anti-FLAG M2, Sigma #F1804), as well as ubiquitinated FLAG-tagged proteins (anti-HA-tag (6E2), CST #2367). Whole-cell lysate (WCL) refers to the normalized input lysate loaded onto DYKDDDDK-sepharose beads.

TUBE1 immunoprecipitation experiments were carried out exactly as described above, except for the fact that 1 mg of normalized HeLa lysate was loaded onto 20 μL TUBE1 agarose (LifeSensors) resin per sample.

Quantitative Real-Time PCR.

MDA-MB-231 cells were plated at 3×10⁵ cells per well in a 6-well dish, allowed to adhere, and were treated with either vehicle (DMSO) or PROTAC (250 nM) for 24 hours. Cell were lysed in 1 mL of TRIzol reagent (ThermoFisher Scientific) per 6-well. Chloroform was added (200 μL) per sample, after which samples were vortexed vigorously for 15 seconds, and centrifuged at 12,000×g for 15 min at 4° C. Total RNA was then precipitated from the aqueous phase by addition of isopropanol (500 μL), followed by centrifugation at 12,000×g for 10 minutes at 4° C. RNA pellet was washed twice with 75% EtOH and allowed to air dry for 15 minutes, after which RNA was dissolved in 25 μL of nuclease-free H₂O. cDNA was synthesized from 4 μg of total RNA per condition according to the manufacturer's protocol (Applied Biosystems) and real-time PCR was performed with 800 nM primers, diluted with 4 μL SYBR Green Reaction Mix (Applied Biosystems). qRT-PCR samples were performed and analyzed in triplicate, from two independent experiments. Beta-tubulin was used for normalization.

Cycloheximide Chase Assay.

MDA-MB-231 cells were plated at 3×10⁵ cells per well in a 6-well dish, allowed to adhere overnight, and switched to serum-free RPMI-1640 (1×) media for 16 hours. Cells were then pre-treated with cycloheximide (Sigma) at 100 μg mL⁻¹ for 1 hour prior to adding either vehicle (DMSO) or PROTAC (250 nM). At the indicated timepoints, cells were immediately placed on ice, rinsed with 1×PBS, lysed, and boiled.

PROTAC Washout Assay.

MDA-MB-231 cells were plated at 1.5×10⁵ cells per well in a 6-well dish and allowed to adhere overnight. On the following day, all cells were treated with either vehicle (DMSO) or PROTAC (250 nM). After 24 hours, cells were either harvested or washed with 1×PBS, trypsinized, and re-plated onto new 6-well dishes in fresh RPMI-1640 (1×) media without additional compound. For these washout conditions, cells were collected every 24 hours until final harvest (72 hours washout).

Molecular Dynamics (MD) Simulations.

The starting coordinates for p38δ came from the crystal structure downloaded from Protein Data Bank (PDB) entry 4EYJ. In order to replace the ligand in this structure with foretinib, the PDB entry 5IA4 was used by overlaying its protein backbone with that of 4EYJ, transferring foretinib to the 4EYJ structure and replacing the original ligand. The obtained p38δ-foretinib complex was subject to the Protein Preparation Wizard of Maestro 2016-3 program available from Schrodinger Inc. (New York City, N.Y.), with which the hydrogen atoms were added, the missing side chains were built, and the protonation states were assigned assuming a pH of 7.0 for the ionizable groups. An energy minimization of the complex was performed for 500 steps.

The starting coordinates for VHL came from the PDB entry 4W9H. The starting coordinates for the p38δ:PROTAC:VHL trimer were prepared as follows. (1) The electrostatic surface was generated for p38δ-ligand complex and VHL-ligand complex, respectively. (2) The VHL-ligand complex was set to have different relative dispositions with respect to the p38δ-ligand complex in a way that the hydrophobic patch of the VHL-ligand surface opposed different hydrophobic patches and grooves of the p38δ-ligand surface, thus producing different starting modes in terms of the relative dispositions between p38δ and VHL. (3) For each starting mode, a linker was built to connect foretinib and VHL ligand and form the full PROTAC. And (4) an energy minimization of 500 steps was performed for each starting point of trimer.

OPLS3 force-field was used throughout the calculation steps. The torsional angle parameters were examined with Force Field Builder program and found that the torsional angles between the amide and cyclopropyl group and between the fluorophenyl group and the oxygen ether atom attached to the quinoline group in foretinib needed corrections; and thus the new torsional profiles were generated to match the profiles given by Jaguar quantum mechanical calculations.

Each starting point of the p38δ:PROTAC:VHL trimer was subject to molecular dynamics (MD) simulation. The system setup was done using System Builder of Maestro program, in which the periodic boundary condition was used; the box shape was cubic with absolute size of each side greater than the largest dimension of the system by 5 Å. The explicit waters were added. The system was neutralized using sodium and chloride ions and salted into 0.15 M ionic strength. The MD was done using Desmond Multisim version 3.8.5.19 which was an eight-stage process: (1) task; (2) simulation of 100 picosecond with Brownian dynamics NVT with T at 10 K, small time-steps and restraints on solute heavy atoms; (3) simulation of 12 picosecond, NVT ensemble, T at 10 K, small time-steps and restraints on solute heavy atoms; (4) simulation of 12 picosecond, NPT ensemble, T at 10 K and restraints on solute heavy atoms; (5) solvation of unfilled pockets; (6) simulation of 12 picosecond up to the target temperature of 310 K, NPT ensemble and restraints on solute heavy atoms; (7) simulation of 24 picosecond, NPT ensemble without restraints at T of 310 K; and finally, (8) production run of 100 nanosecond. During the production run, coordinate frames were saved at every 10 picosecond. The target pressure was set to 1.01325 bar in the related steps.

The post-simulation analysis after each run was done as follows. The last 20 nanosecond of trajectory frames were extracted. A clustering analysis using hierarchical clustering method was performed. The distance between any two members (frames) was the root-mean-square deviation (RMSD) of the solute heavy atoms between the members after overlaying them. The cutoff distance was 2 Å. Every frame was used. The structure closest to the centroid of each cluster was written out as the representative structure of that cluster. The representative structure of the largest cluster for each MD simulation was considered as the representative structure of that simulation run. Such a structure can be considered as the most-visited conformation of that run.

The MD simulation was performed using the g2.2× large instances of Amazon Web Service cloud machines. The Desmond GPU-enabled code was used and mainly run using GPU.

Quantification and Statistical Analysis.

Western blot data in FIGS. 13A, 13B, 13C, 11A, 11B, and 17A were quantified by using the band feature in Image Lab (Bio-Rad), and duplicate values were averaged and analyzed in Graphpad Prism. DC₅₀ and Dₘₐₓ values were fitted using a four parameter with variable slope [inhibitor] versus response with a bottom constraint of zero and reported directly from the Prism output.

A novel bifunctional molecule, which contains a cMet receptor tyrosine kinase recruiting moiety or a p38 (e.g., p38α, p38β, p38γ, and/or p38δ) recruiting moiety and an E3 Ligase recruiting moiety, through PROTAC technology is described. The bifunctional molecules of the present disclosure actively degrade cMet, p38α, p38β, p38γ, and/or p38δ.

The contents of all references, patents, pending patent applications and published patents, cited throughout this application are hereby expressly incorporated by reference.

Those skilled in the art will recognize, or be able to ascertain using no more than routine experimentation, many equivalents to the specific embodiments of the invention described herein. Such equivalents are intended to be encompassed by the following claims. It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

### REFERENCES

1. Hopkins A L & Groom C R (2002) The druggable genome. Nat Rev Drug Discov 1(9):727-730.
2. Fabbro D (2015) 25 years of small molecular weight kinase inhibitors: potentials and limitations. Mol Pharmacol 87(5):766-775.
3. Adjei A A (2006) What is the right dose? The elusive optimal biologic dose in phase I clinical trials. J Clin Oncol 24(25):4054-4055.
4. Fabian M A, et al. (2005) A small molecule-kinase interaction map for clinical kinase inhibitors. Nat Biotechnol 23(3):329-336.
5. Bain J, et al. (2007) The selectivity of protein kinase inhibitors: a further update. Biochem J 408(3):297-315.
6. Fedorov O, et al. (2007) A systematic interaction map of validated kinase inhibitors with Ser/Thr kinases. Proc Natl Acad Sci USA 104(51):20523-20528.
7. Karaman M W, et al. (2008) A quantitative analysis of kinase inhibitor selectivity. Nat Biotechnol 26(1):127-132.
8. Lazo J S & Sharlow E R (2016) Drugging Undruggable Molecular Cancer Targets. Annu Rev Pharmacol Toxicol 56:23-40.
9. Huang X & Dixit V M (2016) Drugging the undruggables: exploring the ubiquitin system for drug development. Cell Res 26(4):484-498.
10. Lai A C & Crews C M (2017) Induced protein degradation: an emerging drug discovery paradigm. Nat Rev Drug Discov 16(2):101-114.
11. Bondeson D P & Crews C M (2017) Targeted Protein Degradation by Small Molecules. Annu Rev Pharmacol Toxicol 57:107-123.
12. Cromm P M & Crews C M (2017) Targeted Protein Degradation: from Chemical Biology to Drug Discovery. Cell Chem Biol 24(9): 1181-1190.
13. Ferguson F M & Gray N S (2018) Kinase inhibitors: the road ahead. Nat Rev Drug Discov 17(5):353-377.
14. Sakamoto K M, et al. (2001) Protacs: chimeric molecules that target proteins to the Skp1-Cullin-F box complex for ubiquitination and degradation. Proc Natl Acad Sci USA 98(15):8554-8559.
15. Toure M & Crews C M (2016) Small-Molecule PROTACS: New Approaches to Protein Degradation. Angew Chem Int Ed Engl 55(6):1966-1973.
16. Ottis P & Crews C M (2017) Proteolysis-Targeting Chimeras: Induced Protein Degradation as a Therapeutic Strategy. ACS Chem Biol 12(4):892-898.
17. Raina K & Crews C M (2017) Targeted protein knockdown using small molecule degraders. Curr Opin Chem Biol 39:46-53.
18. Stanton B Z, Chory E J, & Crabtree G R (2018) Chemically induced proximity in biology and medicine. Science 359(6380).
19. Burslem G M & Crews C M (2017) Small-Molecule Modulation of Protein Homeostasis. Chem Rev 117(17):11269-11301.
20. Churcher I (2018) Protac-Induced Protein Degradation in Drug Discovery: Breaking the Rules or Just Making New Ones? J Med Chem 61(2):444-452.
21. Ottis P, et al. (2017) Assessing Different E3 Ligases for Small Molecule Induced Protein Ubiquitination and Degradation. ACS Chem Biol 12(10):2570-2578.
22. Lu J, et al. (2015) Hijacking the E3 Ubiquitin Ligase Cereblon to Efficiently Target BRD4. Chem Biol 22(6):755-763.
23. Zengerle M, Chan K H, & Ciulli A (2015) Selective Small Molecule Induced Degradation of the BET Bromodomain Protein BRD4. ACS Chem Biol 10(8):1770-1777.
24. Winter G E, et al. (2015) DRUG DEVELOPMENT. Phthalimide conjugation as a strategy for in vivo target protein degradation. Science 348(6241):1376-1381.
25. Hon W C, et al. (2002) Structural basis for the recognition of hydroxyproline in HIF-1 alpha by pVHL. Nature 417(6892):975-978.
26. Min J H, et al. (2002) Structure of an HIF-1alpha-pVHL complex: hydroxyproline recognition in signaling. Science 296(5574): 1886-1889.
27. Schneekloth J S, Jr., et al. (2004) Chemical genetic control of protein levels: selective in vivo targeted degradation. J Am Chem Soc 126(12):3748-3754.
28. Buckley D L, et al. (2015) HaloPROTACS: Use of Small Molecule PROTACs to Induce Degradation of HaloTag Fusion Proteins. ACS Chem Biol 10(8):1831-1837.
29. Bondeson D P, et al. (2015) Catalytic in vivo protein knockdown by small-molecule PROTACs. Nat Chem Biol 11(8):611-617.
30. Raina K, et al. (2016) PROTAC-induced BET protein degradation as a therapy for castration-resistant prostate cancer. Proc Natl Acad Sci USA 113(26):7124-7129.
31. Crew A P, et al. (2018) Identification and Characterization of Von Hippel-Lindau-Recruiting Proteolysis Targeting Chimeras (PROTACs) of TANK-Binding Kinase 1. J Med Chem 61(2):583-598.
32. Burslem G M, et al. (2018) The Advantages of Targeted Protein Degradation Over Inhibition: An RTK Case Study. Cell Chem Biol 25(1):67-77 e63.
33. Lai A C, et al. (2016) Modular PROTAC Design for the Degradation of Oncogenic BCR-ABL. Angew Chem Int Ed Engl 55(2):807-810.
34. Chan K H, Zengerle M, Testa A, & Ciulli A (2018) Impact of Target Warhead and Linkage Vector on Inducing Protein Degradation: Comparison of Bromodomain and Extra-Terminal (BET) Degraders Derived from Triazolodiazepine (JQ1) and Tetrahydroquinoline (I-BET726) BET Inhibitor Scaffolds. J Med Chem 61(2):504-513.
35. Remillard D, et al. (2017) Degradation of the BAF Complex Factor BRD9 by Heterobifunctional Ligands. Angew Chem Int Ed Engl 56(21):5738-5743.
36. Gadd M S, et al. (2017) Structural basis of PROTAC cooperative recognition for selective protein degradation. Nat Chem Biol 13 (5): 514-521.
37. Bondeson D P, et al. (2018) Lessons in PROTAC Design from Selective Degradation with a Promiscuous Warhead. Cell Chem Biol 25(1):78-87 e75.
38. Huang H T, et al. (2018) A Chemoproteomic Approach to Query the Degradable Kinome Using a Multi-kinase Degrader. Cell Chem Biol 25(1):88-99 e86.
39. Hughes S J & Ciulli A (2017) Molecular recognition of ternary complexes: a new dimension in the structure-guided design of chemical degraders. Essays Biochem 61(5):505-516.
40. Cuenda A & Rousseau S (2007) p38 MAP-kinases pathway regulation, function and role in human diseases. Biochim Biophys Acta 1773(8):1358-1375.
41. Coulthard L R, White D E, Jones D L, McDermott M F, & Burchill S A (2009) p38(MAPK): stress responses from molecular mechanisms to therapeutics. Trends Mol Med 15(8):369-379.
42. Kim E K & Choi E J (2010) Pathological roles of MAPK signaling pathways in human diseases. Biochim Biophys Acta 1802(4): 396-405.
43. Cuadrado A & Nebreda A R (2010) Mechanisms and functions of p38 MAPK signalling. Biochem J 429(3):403-417.
44. Cargnello M & Roux P P (2011) Activation and function of the MAPKs and their substrates, the MAPK-activated protein kinases. Microbiol Mol Biol Rev 75(1):50-83.
45. Han J, Lee J D, Bibbs L, & Ulevitch R J (1994) A MAP kinase targeted by endotoxin and hyperosmolarity in mammalian cells. Science 265(5173):808-811.
46. Genovese M C (2009) Inhibition of p38: has the fat lady sung? Arthritis Rheum 60(2):317-320.
47. Astolfi A, Manfroni G, Cecchetti V, & Barreca M L (2018) A Comprehensive Structural Overview of p38alpha Mitogen-Activated Protein Kinase in Complex with ATP-Site and Non-ATP-Site Binders. Chem Med Chem 13(1):7-14.
48. Escos A, Risco A, Alsina-Beauchamp D, & Cuenda A (2016) p38gamma and p38delta Mitogen Activated Protein Kinases (MAPKs), New Stars in the MAPK Galaxy. Front Cell Dev Biol 4:31.
49. Alevy Y G, et al. (2012) IL-13-induced airway mucus production is attenuated by MAPK13 inhibition. J Clin Invest 122(12):4555-4568.
50. Yurtsever Z, Scheaffer S M, Romero A G, Holtzman M J, & Brett T J (2015) The crystal structure of phosphorylated MAPK13 reveals common structural features and differences in p38 MAPK family activation. Acta Crystallogr D Biol Crystallogr 71(Pt 4): 790-799.
51. Yurtsever Z, et al. (2016) First comprehensive structural and biophysical analysis of MAPK13 inhibitors targeting DFG-in and DFG-out binding modes. Biochim Biophys Acta 1860(11 Pt A): 2335-2344.
52. Anastassiadis T, Deacon S W, Devarajan K, Ma H, & Peterson J R (2011) Comprehensive assay of kinase catalytic activity reveals features of kinase inhibitor selectivity. Nat Biotechnol 29(11):1039-1045.
53. Douglass E F, Jr., Miller C J, Sparer G, Shapiro H, & Spiegel D A (2013) A comprehensive mathematical model for three-body binding equilibria. J Am Chem Soc 135(16):6092-6099.
54. Meng L, et al. (1999) Epoxomicin, a potent and selective proteasome inhibitor, exhibits in vivo antiinflammatory activity. Proc Natl Acad Sci USA 96(18):10403-10408.
55. Soucy T A, et al. (2009) An inhibitor of NEDD8-activating enzyme as a new approach to treat cancer. Nature 458(7239):732-736.
56. Thrower J S, Hoffman L, Rechsteiner M, & Pickart C M (2000) Recognition of the polyubiquitin proteolytic signal. EMBO J 19(1):94-102.
57. Singh S K, et al. (2016) Synthetic Uncleavable Ubiquitinated Proteins Dissect Proteasome Deubiquitination and Degradation, and Highlight Distinctive Fate of Tetraubiquitin. J Am Chem Soc 138(49):16004-16015.
58. Davis M I, et al. (2011) Comprehensive analysis of kinase inhibitor selectivity. Nat Biotechnol 29(11):1046-1051.
59. Savitski M M, et al. (2014) Tracking cancer drugs in living cells by thermal profiling of the proteome. Science 346(6205): 1255784.
60. Mateus A, Maatta T A, & Savitski M M (2016) Thermal proteome profiling: unbiased assessment of protein state through heat-induced stability changes. Proteome Sci 15:13.
61. Jafari R, et al. (2014) The cellular thermal shift assay for evaluating drug target interactions in cells. Nat Protoc 9(9):2100-2122.
62. Hjerpe R, et al. (2009) Efficient protection and isolation of ubiquitylated proteins using tandem ubiquitin-binding entities. EMBO Rep 10(11): 1250-1258.
63. Maniaci C, et al. (2017) Homo-PROTACs: bivalent small-molecule dimerizers of the VHL E3 ubiquitin ligase to induce self-degradation. Nat Commun 8(1):830.
64. Tanoue T, Adachi M, Moriguchi T, & Nishida E (2000) A conserved docking motif in MAP kinases common to substrates, activators and regulators. Nat Cell Biol 2(2):110-116.
65. Tanoue T & Nishida E (2002) Docking interactions in the mitogen-activated protein kinase cascades. Pharmacol Ther 93(2-3):193-202.
66. Goldsmith E J (2011) Three-dimensional docking in the MAPK p38alpha. Sci Signal 4(204):pe47.
67. Garai A, et al. (2012) Specificity of linear motifs that bind to a common mitogen-activated protein kinase docking groove. Sci Signal 5(245):ra74.
68. Watterson D M, et al. (2013) Development of Novel In Vivo Chemical Probes to Address CNS Protein Kinase Involvement in Synaptic Dysfunction. PLoS One 8(6):e66226.
69. Sumara G, et al. (2009) Regulation of PKD by the MAPK p38delta in insulin secretion and glucose homeostasis. Cell 136(2):235-248.

What is claimed is:

1. A bifunctional compound having the chemical structure:
PTM-L-ULM,
or a pharmaceutically acceptable salt, enantiomer, stereoisomer, solvate, polymorph or prodrug thereof,
wherein:
the ULM is a small molecule E3 ubiquitin ligase binding moiety that binds an E3 ubiquitin ligase;
the L is a bond or a chemical linking moiety connecting the ULM and the PTM; and
the PTM is selected from: 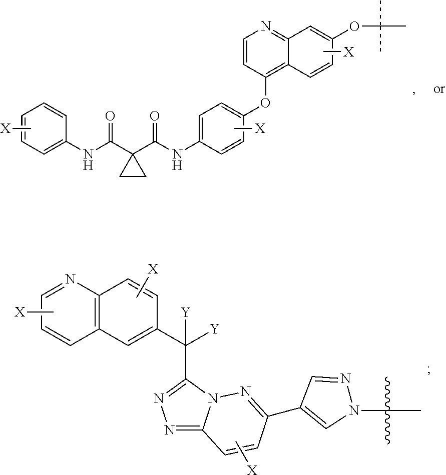
each X is independently Cl, F, Br, H, CN, Me, OMe, or OCF₃ and;
each Y is independently F or H.

2. The compound according to claim 1, wherein the E3 ubiquitin ligase binding moiety that targets an E3 ubiquitin ligase selected from the group consisting of Von Hippel-Lindau (VLM), cereblon (CLM), mouse double-minute homolog2 (MLM), and IAP (ILM).

3. The compound according to claim 1, wherein ULM is a Von Hippel-Lindau (VHL) ligase-binding moiety (VLM) with a chemical structure represented by: 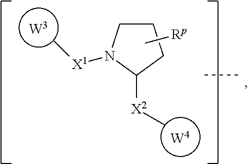
wherein:
X¹, X² are each independently selected from the group of a bond, O, NR^{Y3}, CR^{Y3}R^{Y4}, C═O, C═S, SO, and SO₂;
R^{Y3}, R^{Y4} are each independently selected from the group of H, linear or branched C₁₋₆ alkyl, optionally substituted by 1 or more halo, optionally substituted C₁₋₆ alkoxyl (e.g., optionally substituted by 0-3 R^{P} groups);
R^{P} is 0, 1, 2, or 3 groups, each independently selected from the group H, halo, —OH, C₁₋₃ alkyl, C═O;
W³ is selected from the group of an optionally substituted T, an optionally substituted -T-N(R^{1a}R^{1b})X³, optionally substituted -T-N(R^{1a}R^{1b}), optionally substituted -T-Aryl, an optionally substituted -T-Heteroaryl, an optionally substituted T-biheteroaryl, an optionally substituted -T-Heterocycle, an optionally substituted -T-biheterocycle, an optionally substituted —NR¹-T-Aryl, an optionally substituted —NR¹-T-Heteroaryl or an optionally substituted —NR¹-T-Heterocycle;
X³ is C═O, R¹, R^{1a}, R^{1b} are independently selected from the group of H, linear or branched C₁-C₆ alkyl group optionally substituted by 1 or more halo or —OH groups, R^{Y3}C═O, R^{Y3}C═S, R^{Y3}SO, R^{Y3}SO₂, N(R^{Y3}R^{Y4})C═O, N(R^{Y3}R^{Y4})C═S, N(R^{Y3}R^{Y4})SO, and N(R^{Y3}R^{Y4})SO₂;
T is selected from the group of an optionally substituted alkyl, —(CH₂)ₙ— group, wherein each one of the methylene groups is optionally substituted with one or two substituents selected from the group of halogen, methyl, a linear or branched C₁-C₆ alkyl group optionally substituted by 1 or more halogen or —OH groups or an amino acid side chain optionally substituted; and
n is 0 to 6,
W⁴ is 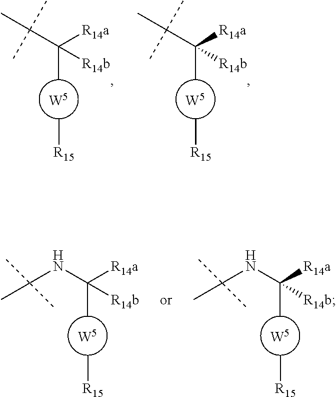
R₁₄ₐ, R_{14b}, are each independently selected from the group of H, haloalkyl, or optionally substituted alkyl;
W⁵ is selected from the group of a phenyl or a 5-10 membered heteroaryl,
R₁₅ is selected from the group of H, halogen, CN, OH, NO₂, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐ SO₂R_{14b}, optionally substituted alkyl, optionally substituted haloalkyl, optionally substituted haloalkoxy; optionally substituted aryl; optionally substituted heteroaryl; optionally substituted cycloalkyl; or optionally substituted cycloheteroalkyl;
and wherein the dashed line indicates the site of attachment of at least one PTM, another ULM (ULM′) or a chemical linker moiety coupling at least one PTM or a ULM′ or both to ULM.

4. The compound according to claim 1, wherein ULM is a Von Hippel-Lindau (VHL) ligase-binding moiety (VLM) with a chemical structure represented by: 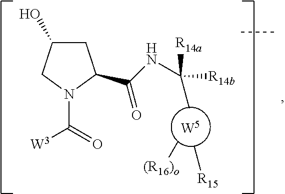
wherein:
W³ is selected from the group of an optionally substituted aryl, optionally substituted heteroaryl, or 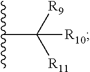
R₉ and R₁₀ are independently hydrogen, optionally substituted alkyl, optionally substituted cycloalkyl, optionally substituted hydroxyalkyl, optionally substituted heteroaryl, or haloalkyl, or R₉, R₁₀, and the carbon atom to which they are attached form an optionally substituted cycloalkyl;
R₁₁ is selected from the group of an optionally substituted heterocyclic, optionally substituted alkoxy, optionally substituted heteroaryl, optionally substituted aryl, 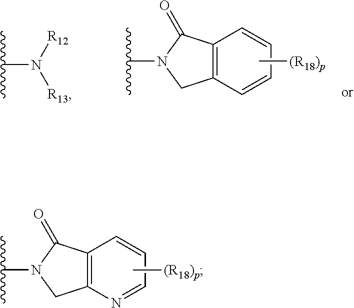
R₁₂ is selected from the group of H or optionally substituted alkyl;
R₁₃ is selected from the group of H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R₁₄ₐ, R_{14b}, are each independently selected from the group of H, haloalkyl, or optionally substituted alkyl;
W⁵ is selected from the group of a phenyl or a 5-10 membered heteroaryl,
R₁₅ is selected from the group of H, halogen, CN, OH, NO₂, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐ SO₂R_{14b}, optionally substituted alkyl, optionally substituted haloalkyl, optionally substituted haloalkoxy; optionally substituted aryl; optionally substituted heteroaryl; optionally substituted cycloalkyl; or optionally substituted cycloheteroalkyl;
R₁₆ is independently selected from the group of halo, optionally substituted alkyl, optionally substituted haloalkyl, hydroxy, or optionally substituted haloalkoxy;
o is 0, 1, 2, 3, or 4;
R₁₈ is independently selected from the group of H, halo, optionally substituted alkoxy, cyano, optionally substituted alkyl, haloalkyl, haloalkoxy or a linker; and
p is 0, 1, 2, 3, or 4, and wherein the dashed line indicates the site of attachment of at least one PTM, another ULM (ULM′) or a chemical linker moiety coupling at least one PTM or a ULM′ or both to ULM.

5. The compound according to claim 1, wherein the ULM has a chemical structure selected from the group of: 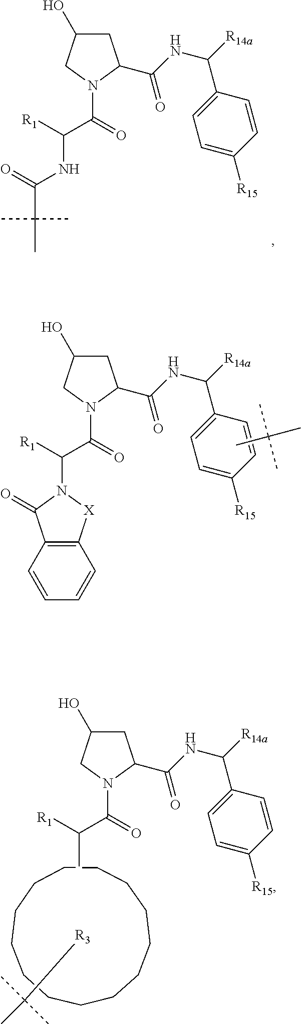
and
wherein:
R₁ is H, ethyl, isopropyl, tert-butyl, sec-butyl, cyclopropyl, cyclobutyl, cyclopentyl, or cyclohexyl; optionally substituted alkyl, optionally substituted hydroxyalkyl, optionally substituted heteroaryl, or haloalkyl;
R₁₄ₐ is H, haloalkyl, optionally substituted alkyl, methyl, fluoromethyl, hydroxymethyl, ethyl, isopropyl, or cyclopropyl;
R₁₅ is selected from the group consisting of H, halogen, CN, OH, NO₂, optionally substituted heteroaryl, optionally substituted aryl; optionally substituted alkyl; optionally substituted haloalkyl; optionally substituted haloalkoxy; optionally substituted cycloalkyl; or optionally substituted cycloheteroalkyl;
X is C, CH₂, or C═O
R₃ is absent or an optionally substituted 5 or 6 membered heteroaryl; and
the dashed line indicates the site of attachment of at least one PTM, another ULM (ULM′) or a chemical linker moiety coupling at least one PTM or a ULM′ or both to the ULM.

6. The compound according to claim 1, wherein the ULM comprises a group according to the chemical structure: 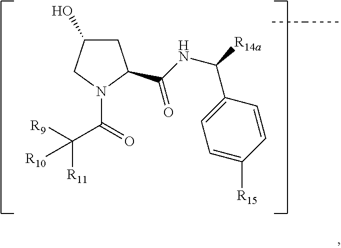
wherein:
R₁₄ₐ is H, haloalkyl, optionally substituted alkyl, methyl, fluoromethyl, hydroxymethyl, ethyl, isopropyl, or cyclopropyl;
R9 is H;
R10 is H, ethyl, isopropyl, tert-butyl, sec-butyl, cyclopropyl, cyclobutyl, cyclopentyl, or cyclohexyl;
R11 is 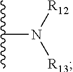
optionally substituted heteroaryl, 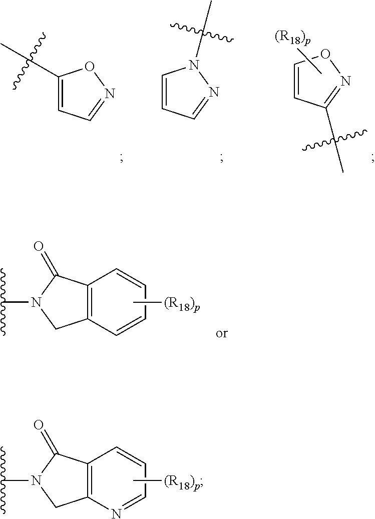
p is 0, 1, 2, 3, or 4; and
each R₁₈ is independently halo, optionally substituted alkoxy, cyano, optionally substituted alkyl, haloalkyl, haloalkoxy or a linker;
R12 is H, C═O
R13 is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl,
R₁₅ is selected from the group consisting of H, halogen, Cl, CN, OH, NO₂, optionally substituted heteroaryl, optionally substituted aryl; 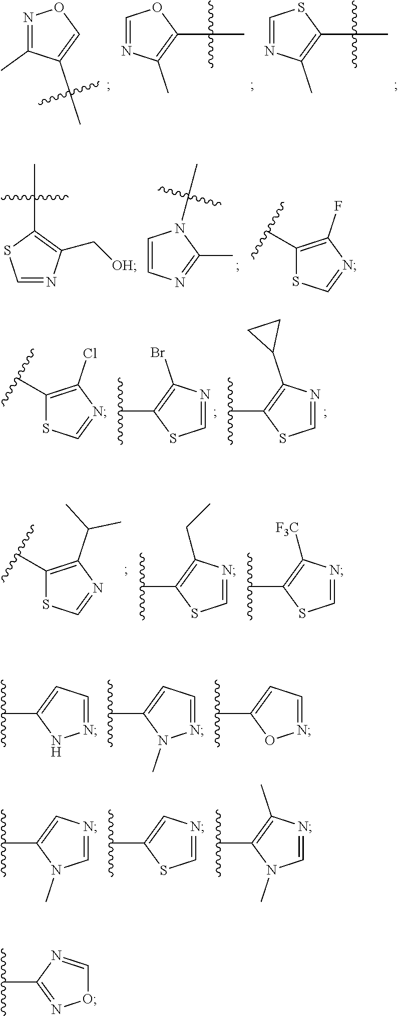
and wherein the dashed line indicates the site of attachment of at least one PTM, another ULM (ULM′) or a chemical linker moiety coupling at least one PTM or a ULM′ or both to the ULM.

7. The compound of claim 1, wherein the ULM is represented by the chemical structure: 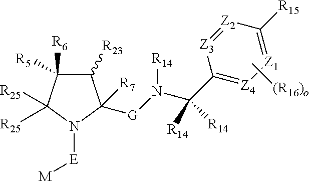
wherein:
each R₅ and R₆ is independently —OH, —SH, or optionally substituted alkyl or R₅, R₆, and the carbon atom to which they are attached form a carbonyl;
R₇ is H or optionally substituted alkyl;
E is a bond, C═O, or C═S;
G is a bond, optionally substituted alkyl, —COOH or C=J;
J is O or N—R₈;
R₈ is H, CN, optionally substituted alkyl or optionally substituted alkoxy;
M is optionally substituted aryl, optionally substituted heteroaryl, optionally substituted heterocyclic or 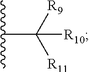
each R₉ and R₁₀ is independently H; optionally substituted alkyl, optionally substituted cycloalkyl, optionally substituted hydroxyalkyl, optionally substituted thioalkyl, a disulphide linked ULM, optionally substituted heteroaryl, or haloalkyl; or R₉, R₁₀, and the carbon atom to which they are attached form an optionally substituted cycloalkyl;
R₁₁ is optionally substituted heterocyclic, optionally substituted alkoxy, optionally substituted heteroaryl, optionally substituted aryl, or 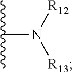
R₁₂ is H or optionally substituted alkyl;
R₁₃ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl; optionally substituted (oxoalkyl)carbamate,
each R₁₄ is independently H, haloalkyl, optionally substituted cycloalkyl, optionally substituted alkyl or optionally substituted heterocycloalkyl;
R₁₅ is H, optionally substituted heteroaryl, haloalkyl, optionally substituted aryl, optionally substituted alkoxy, or optionally substituted heterocyclyl;
each R₁₆ is independently halo, optionally substituted alkyl, optionally substituted haloalkyl, CN, or optionally substituted haloalkoxy;
each R₂₅ is independently H or optionally substituted alkyl; or both R₂₅ groups can be taken together to form an oxo or optionally substituted cycloalkyl group;
R₂₃ is H or OH;
Z₁, Z₂, Z₃, and Z₄ are independently C or N; and
o is 0, 1, 2, 3, or 4, or a pharmaceutically acceptable salt, stereoisomer, solvate or polymorph thereof.

8. The compound according to claim 1, wherein the ULM is a cereblon E3 ligase-binding moiety (CLM) selected from the group consisting of a thalidomide, pomalidomide, analogs thereof, isosteres thereof, or derivatives thereof.

9. The compound according to claim 8, wherein the CLM has a chemical structure represented by: 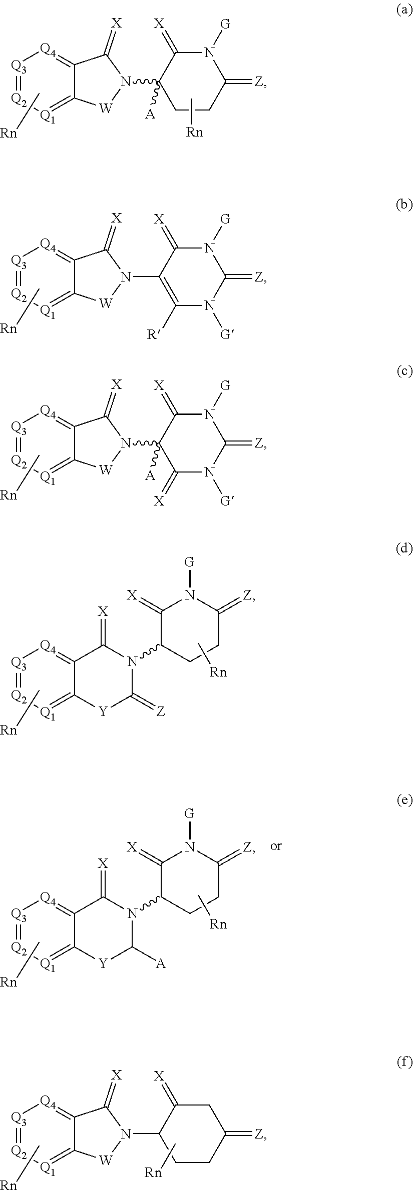
wherein:
W is selected from the group consisting of CH₂, CHR, C═O, SO₂, NH, and N-alkyl;
each X is independently selected from the group consisting of O, S, and H₂,
Y is selected from the group consisting of CH₂, —C═CR′, NH, N-alkyl, N-aryl, N-hetaryl, N-cycloalkyl, N-heterocyclyl, O, and S;
Z is selected from the group consisting of O, S, and H₂;
G and G′ are independently selected from the group consisting of H, optionally substituted linear or branched alkyl, OH, R′OCOOR, R′OCONRR″, CH₂-heterocyclyl optionally substituted with R′, and benzyl optionally substituted with R′;
Q₁, Q₂, Q₃, and Q₄ represent a carbon C substituted with a group independently selected from R′, N or N-oxide;
A is independently selected from the group H, optionally substituted linear or branched alkyl, cycloalkyl, Cl and F;
R comprises —CONR′R″, —OR′, —NR′R″, —SR′, —SO₂R′, —SO₂NR′R″, —CR′R″—, —CR′NR′R″—, (—CR′O)_{n′}R″, -aryl, -hetaryl, optionally substituted linear or branched alkyl, -cycloalkyl, -heterocyclyl, —P(O)(OR′)R″, —P(O)R′R″, —OP(O)(OR′)R″, —OP(O)R′R″, —Cl, —F, —Br, —I, —CF₃, —CN, —NR′SO₂NR′R″, —NR′CONR′R″, —CONR′COR″, —NR′C(═N—CN)NR′R″, —C(═N—CN)NR′R″, —NR′C(═N—CN)R″, —NR′C(═C—NO₂)NR′R″, —SO₂NR′COR″, —NO₂, —CO₂R′, —C(C═N—OR′)R″, —CR′═CR′R″, —CCR′, —S(C═O)(C═N—R′)R″, —SF₅ and —OCF₃;
R′ and R″ are independently selected from the group consisting of a bond, H, alkyl, cycloalkyl, aryl, heteroaryl, heterocyclic, —C(═O)R, heterocyclyl, each of which is optionally substituted; [symbol]
represents a bond that may be stereospecific ((R) or (S)) or non-stereospecific; and
Rₙ comprises from 1 to 4 independently selected functional groups or atoms, for example, O, OH, N, C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy,
wherein n′ is an integer from 1-10, and wherein
when n is 1, Rₙ is modified to be covalently joined to the linker group (L), and
when n is 2, 3, or 4, then one Rₙ is modified to be covalently joined to the linker group (L), and any other Rₙ is optionally modified to be covalently joined to a PTM, a CLM, a second CLM having the same chemical structure as the CLM, a CLM′, a second linker, or any multiple or combination thereof.

10. The compound according to claim 8, wherein the CLM has a chemical structure represented by: 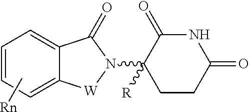
wherein:
W is independently selected from the group CH₂, C═O, NH, and N-alkyl;
R is independently selected from a H, methyl, optionally substituted linear or branched alkyl; [symbol]
represents a bond that may be stereospecific ((R) or (S)) or non-stereospecific; and
Rn comprises from 1 to 4 independently selected functional groups or atoms, for example, O, OH, N, C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy, and optionally, one of which is modified to be covalently joined to a PTM, a chemical linker group (L), a CLM (or CLM′) or combination thereof.

11. The compound according to claim 8, wherein the CLM has a chemical structure represented by: 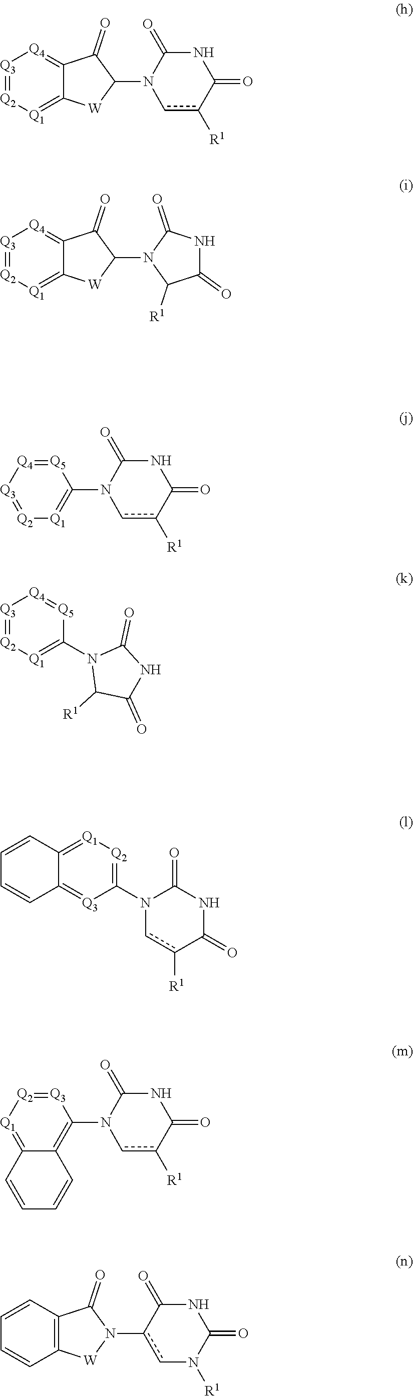 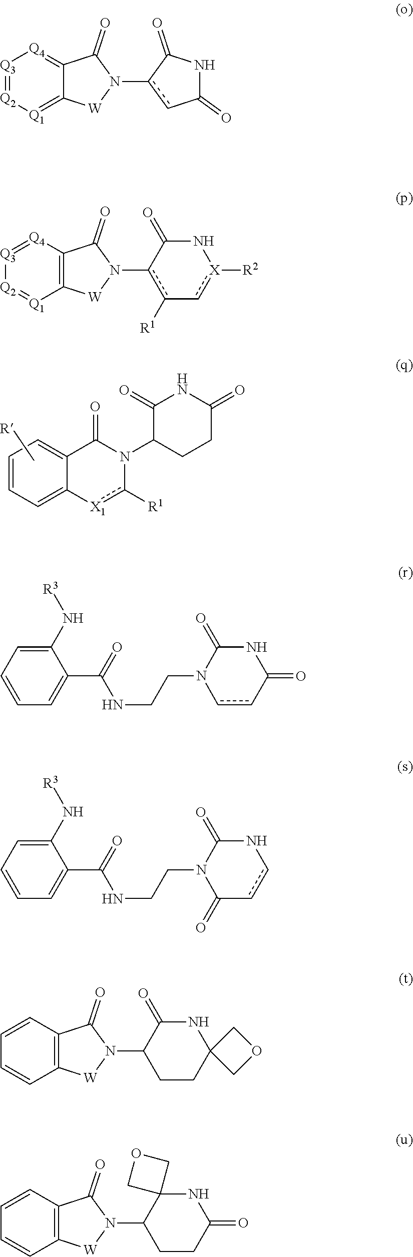 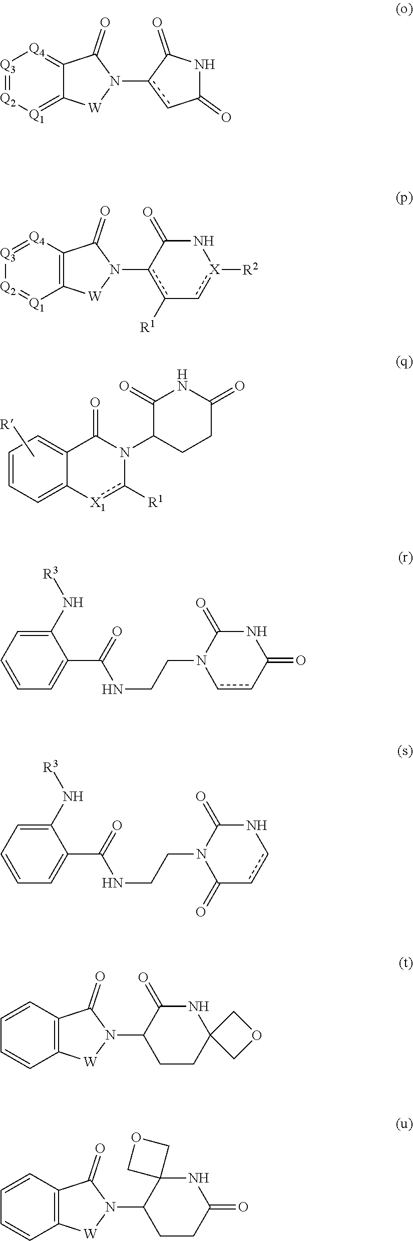 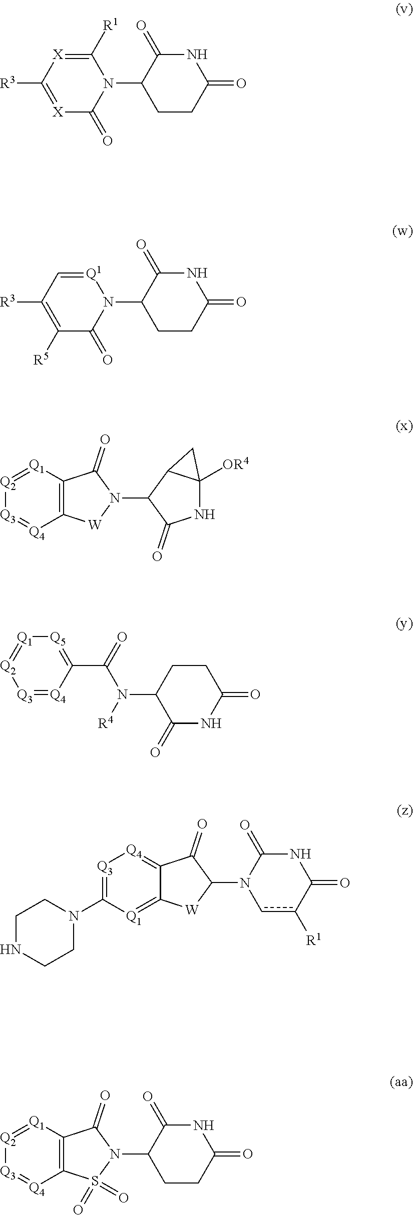 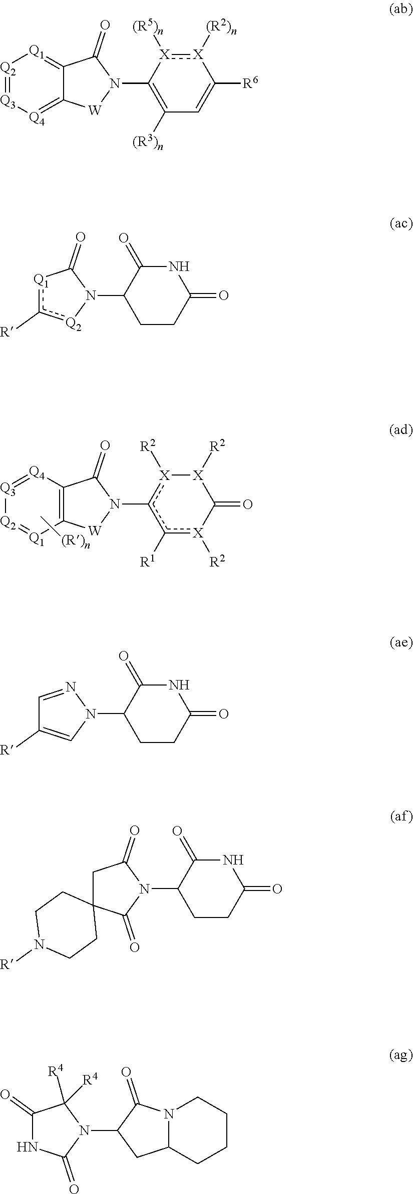 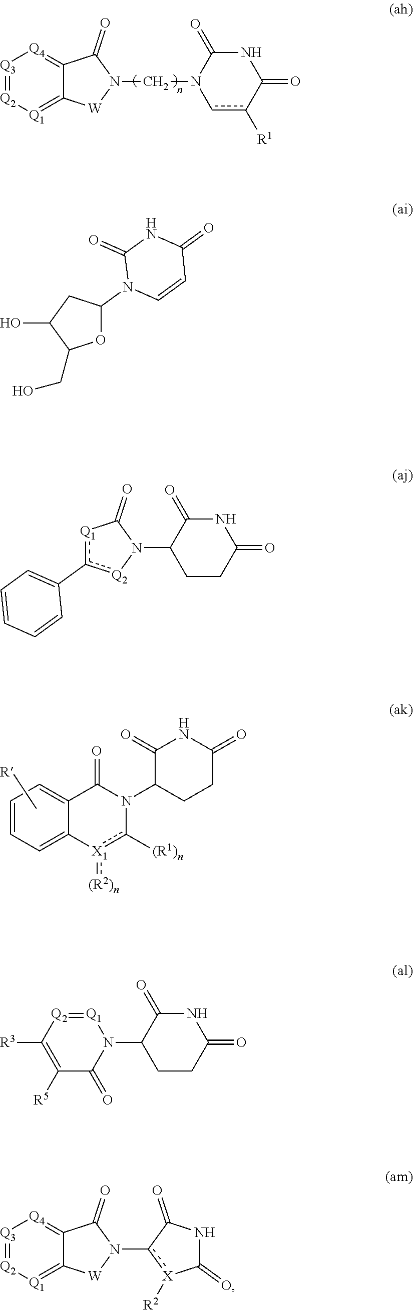
wherein:
W is independently selected from CH₂, CHR, C═O, SO₂, NH, and N-alkyl;
Q₁, Q₂, Q₃, Q₄, Q₅ are each independently represent a carbon C or N substituted with a group independently selected from R′, N or N-oxide;
R¹ is selected from absent, H, OH, CN, C1-C3 alkyl, C═O;
R² is selected from the group absent, H, OH, CN, C1-C3 alkyl, CHF₂, CF₃, CHO, C(═O)NH₂;
R³ is selected from H, alkyl (e.g., C1-C6 or C1-C3 alkyl), substituted alkyl (e.g., substituted C1-C6 or C1-C3 alkyl), alkoxy (e.g., C1-C6 or C1-C3 alkoxyl), substituted alkoxy (e.g., substituted C1-C6 or C1-C3 alkoxyl);
R⁴ is selected from H, alkyl, substituted alkyl;
R⁵ and R⁶ are each independently H, halogen, C(═O)R′, CN, OH, CF₃;
X is C, CH, C═O, or N;
X₁ is C═O, N, CH, or CH₂;
R′ is selected from H, halogen, amine, alkyl (e.g., C1-C3 alkyl), substituted alkyl (e.g., substituted C1-C3 alkyl), alkoxy (e.g., C1-C3 alkoxyl), substituted alkoxy (e.g., substituted C1-C3 alkoxyl), NR²R³, C(═O)OR², optionally substituted phenyl;
n is 0-4; [symbol]
is a single or double bond; and
the CLM is covalently joined to a PTM, a chemical linker group (L), a ULM, CLM (or CLM′) or combination thereof.

12. The compound according to claim 1, wherein the ULM is a (MDM2) binding moiety (MLM) with a chemical moiety selected from the group consisting of a substituted imidazolines, a substituted spiro-indolinones, a substituted pyrrolidines, a substituted piperidinones, a substituted morpholinones, a substituted pyrrolopyrimidines, a substituted imidazolopyridines, a substituted thiazoloimidazoline, a substituted pyrrolopyrrolidinones, and a substituted isoquinolinones.

13. The compound according to claim 1, wherein the ULM is a IAP E3 ubiquitin ligase binding moiety (ILM) comprising the amino acids alanine (A), valine (V), proline (P), and isoleucine (I) or their unnatural mimetics.

14. The compound according to claim 1, wherein the ULM is a IAP E3 ubiquitin ligase binding moiety (ILM) comprising a AVPI tetrapeptide fragment or derivative thereof.

15. The compound according to claim 1, wherein the linker (L) comprises a chemical structural unit represented by the formula:
-(A)_{q}-, wherein
(A)_{q} is a group which is connected to at least one of ULM moiety, PTM moiety, or both;
q is an integer greater than or equal to 1;
each A is independently selected from the group consisting of, a bond, CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, siR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₃₋₁₁heteocyclyl optionally substituted with 0-6 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R^{L5} groups; and
R^{L1}, R^{L2}, R^{L3} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NH SO₂NH(C₁₋₈alkyl), NH SO₂N(C₁₋₈alkyl)₂, NH SO₂NH₂.

16. The compound according to claim 15, wherein the linker (L) comprises a group represented by a general structure selected from the group consisting of: 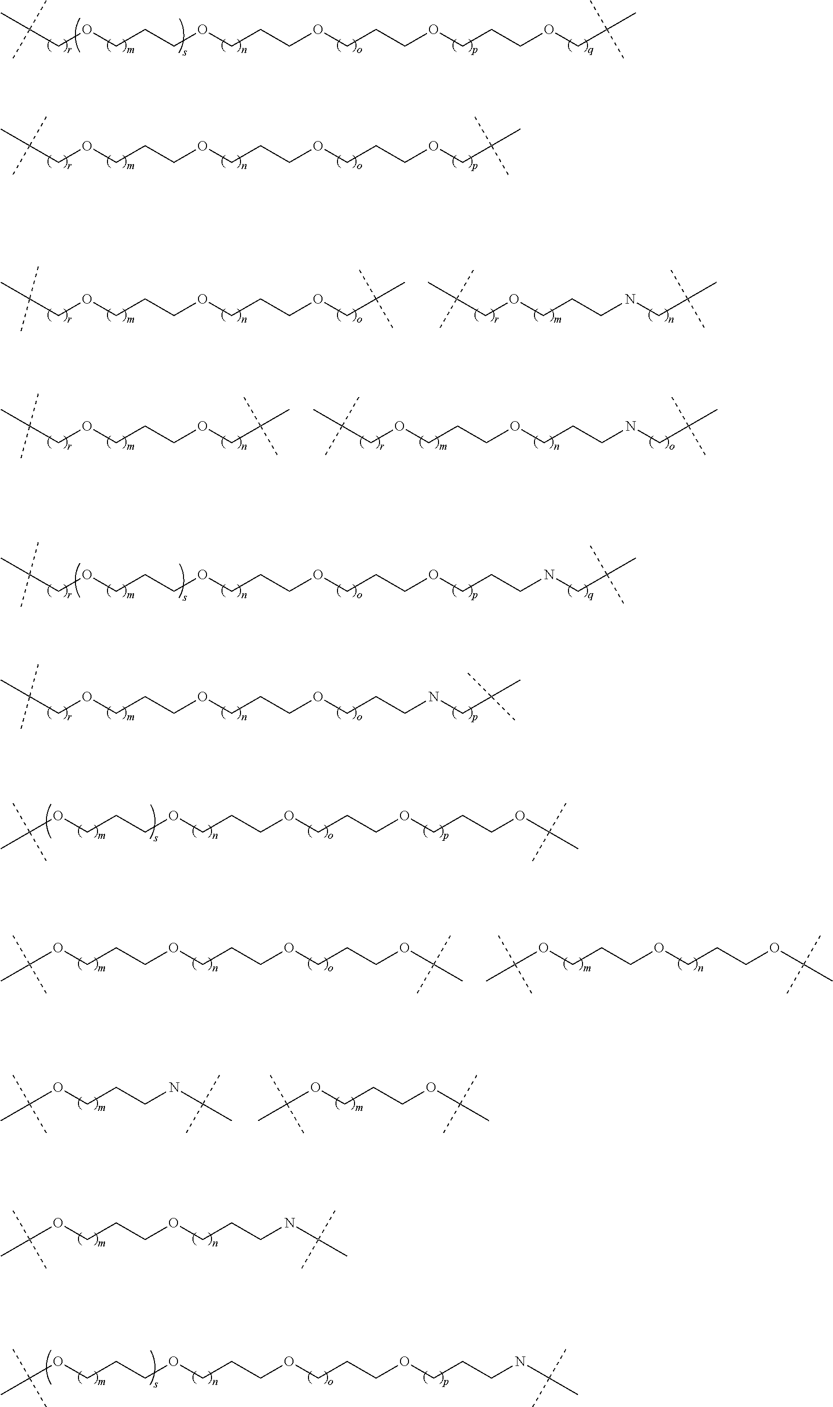 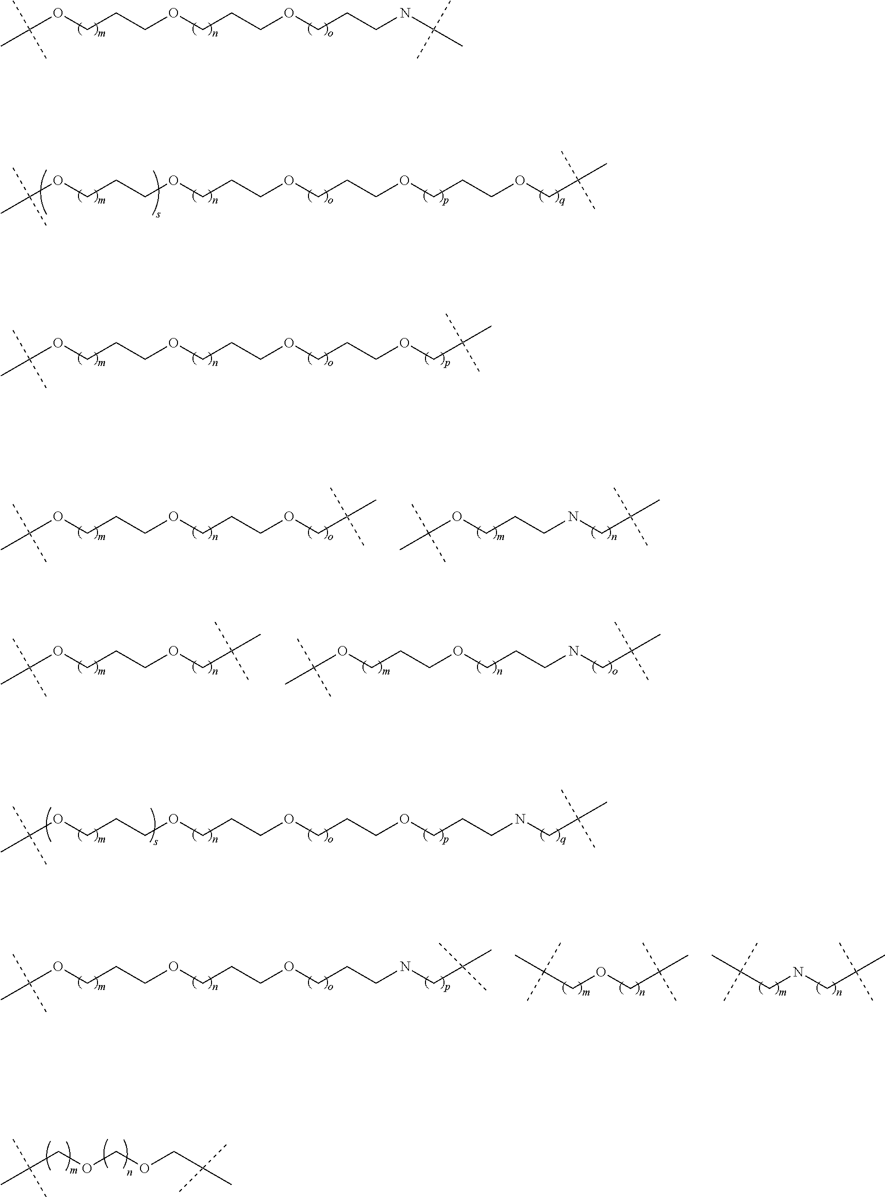
N(R)—(CH2)ₘ—O(CH2)ₙ—O(CH2)ₒ—O(CH2)ₚ—O(CH2)_{q}—O(CH2)ᵣ-OCH2-, —O—(CH2)ₘ—O(CH2)ₙ—O(CH2)ₒ—O(CH2)ₚ—O(CH2)_{q}—O(CH2)ᵣ-OCH2-, —O—(CH2)ₘ—O(CH2)ₙ—O(CH2)ₒ—O(CH2)ₚ—O(CH2)_{q}—O(CH2)ᵣ—O—;
—N(R)—(CH2)ₘ—O(CH2)ₙ—O(CH2)ₒ—O(CH2)ₚ—O(CH2)_{q}—O(CH2)ᵣ—O—;
—(CH2)ₘ—O(CH2)ₙ—O(CH2)ₒ—O(CH2)ₚ—O(CH2)_{q}—O(CH2)ᵣ—O—;
—(CH2)ₘ—O(CH2)ₙ—O(CH2)ₒ—O(CH2)ₚ—O(CH2)_{q}—O(CH2)ᵣ-OCH2-; 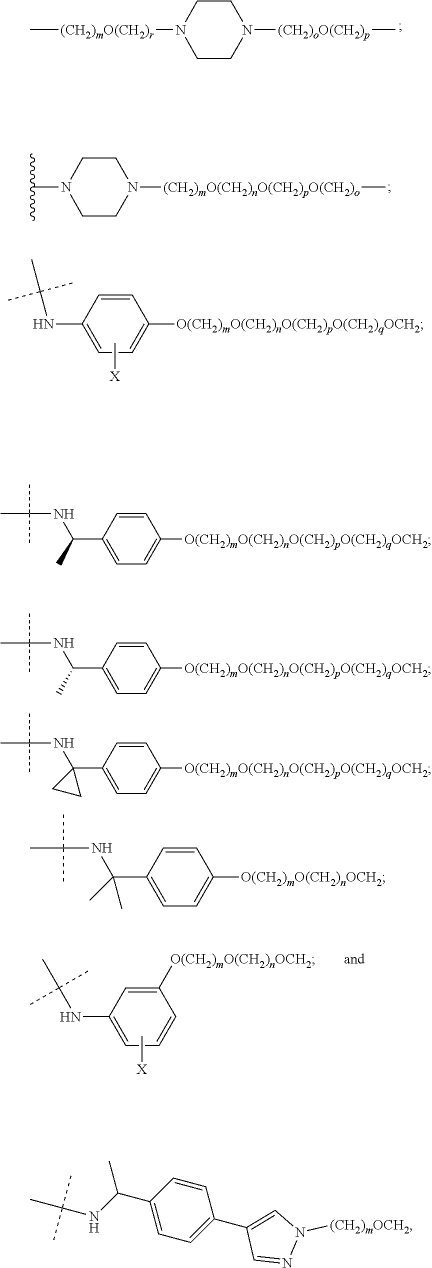
wherein each m, n, o, p, q, r, and s, are independently 0, 1, 2, 3, 4, 5, 6, 7, 8, 9, 10, 11, 12, 13, 14, 15, 16, 17, 18, 19, or 20 with the proviso that when the number is zero, there is no N—O or O—O bond, R is selected from the group H, methyl and ethyl, and X is selected from the group H and F;

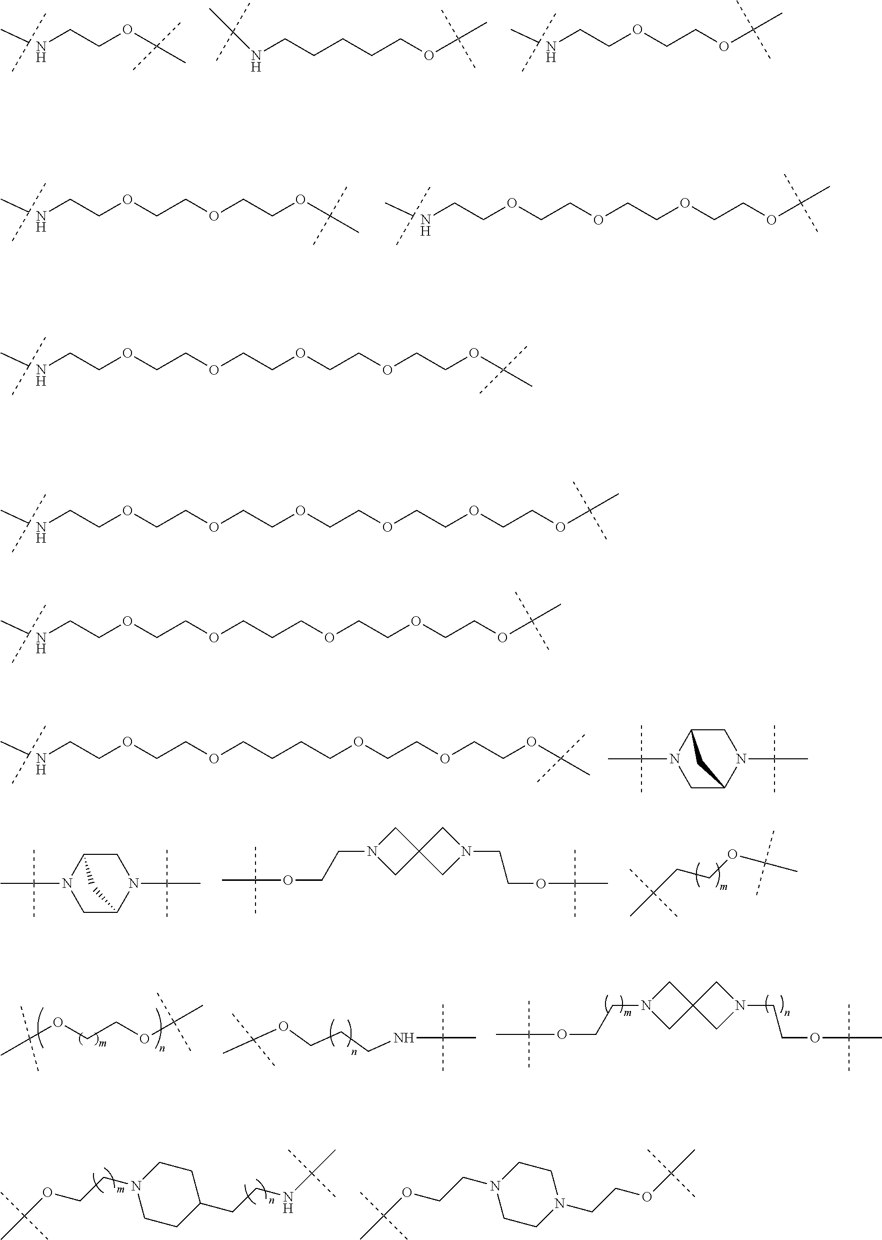

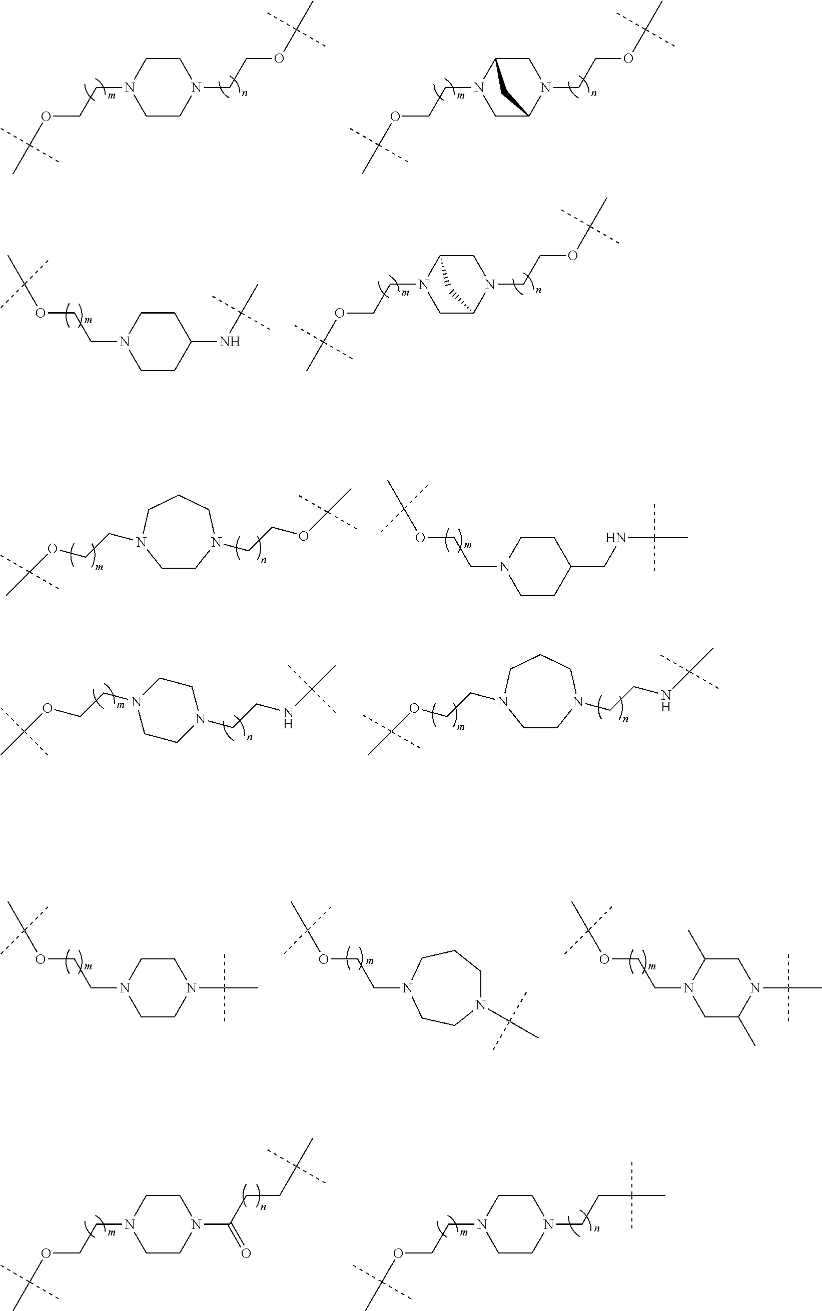

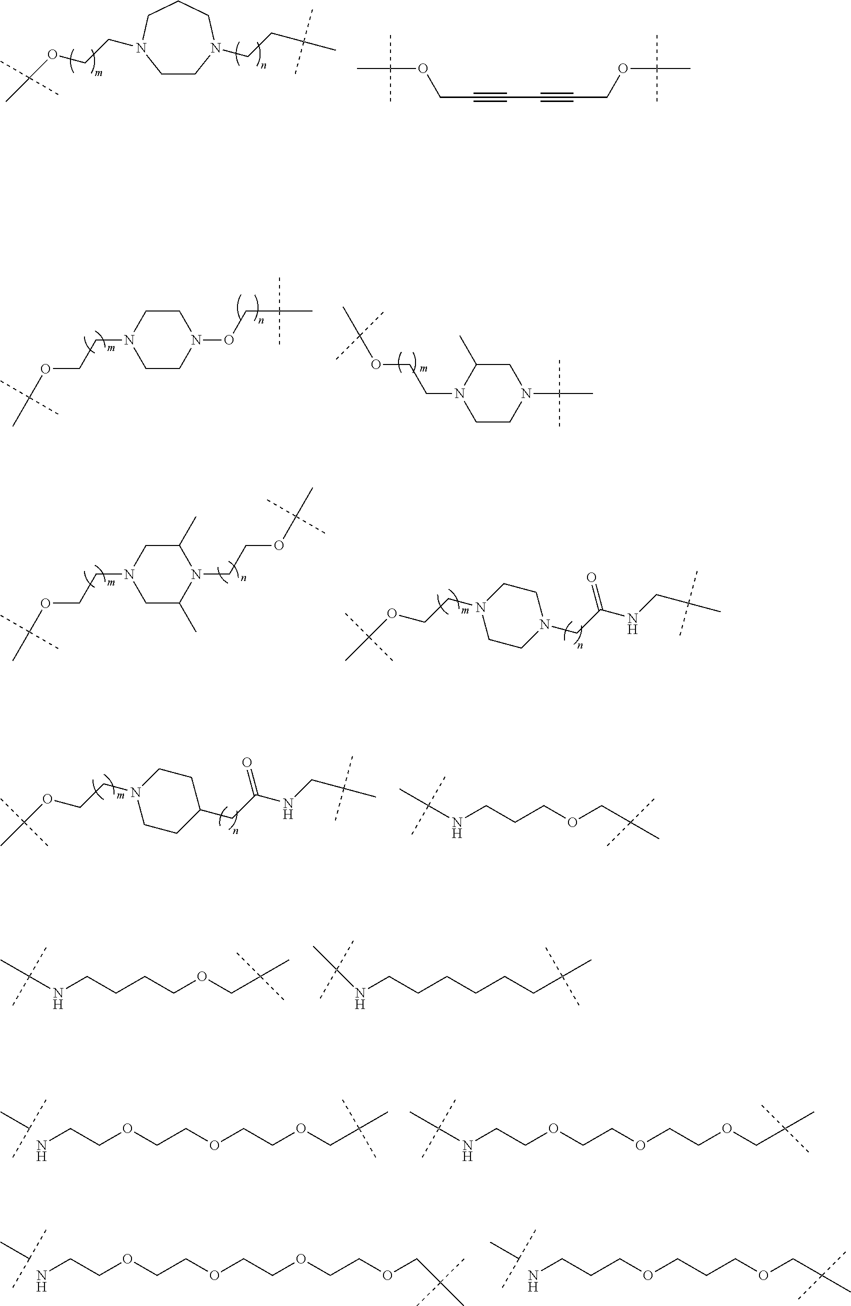

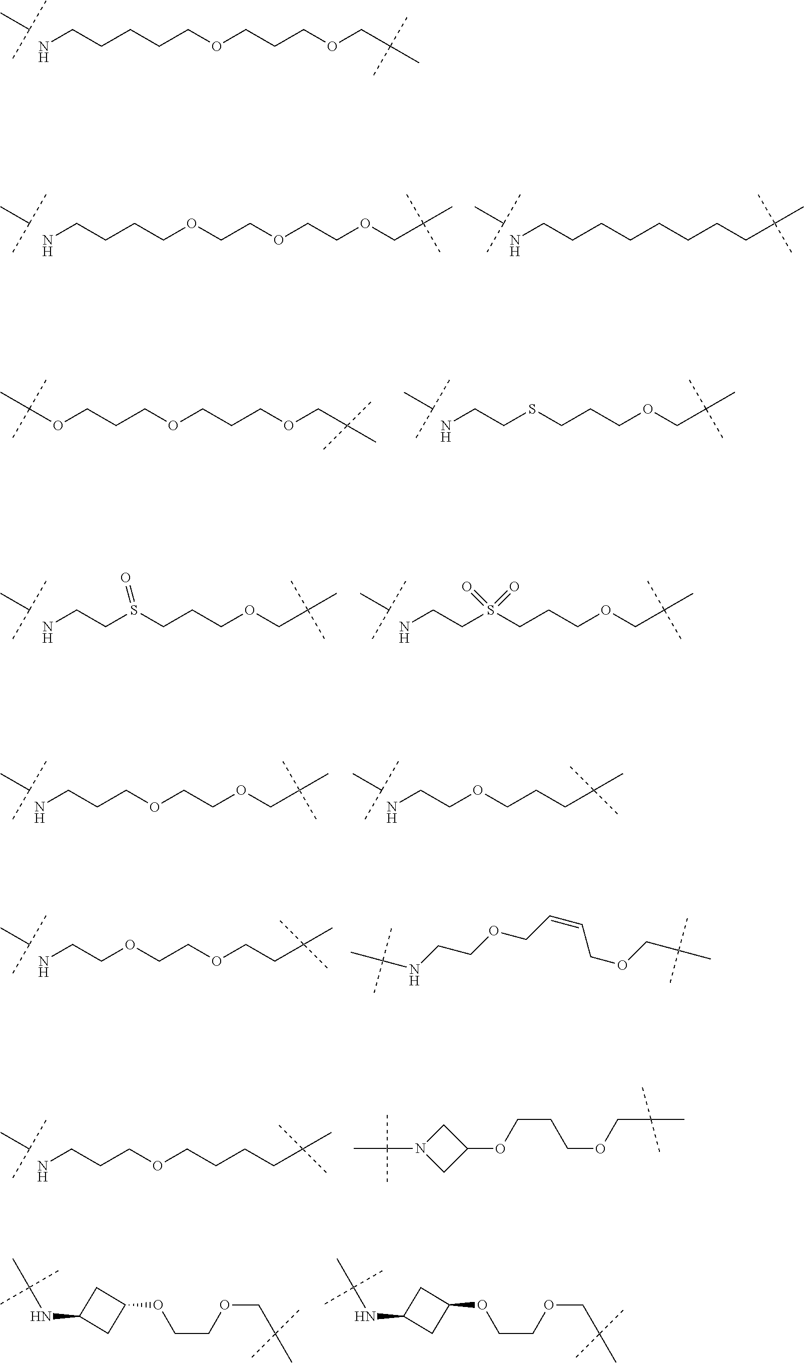

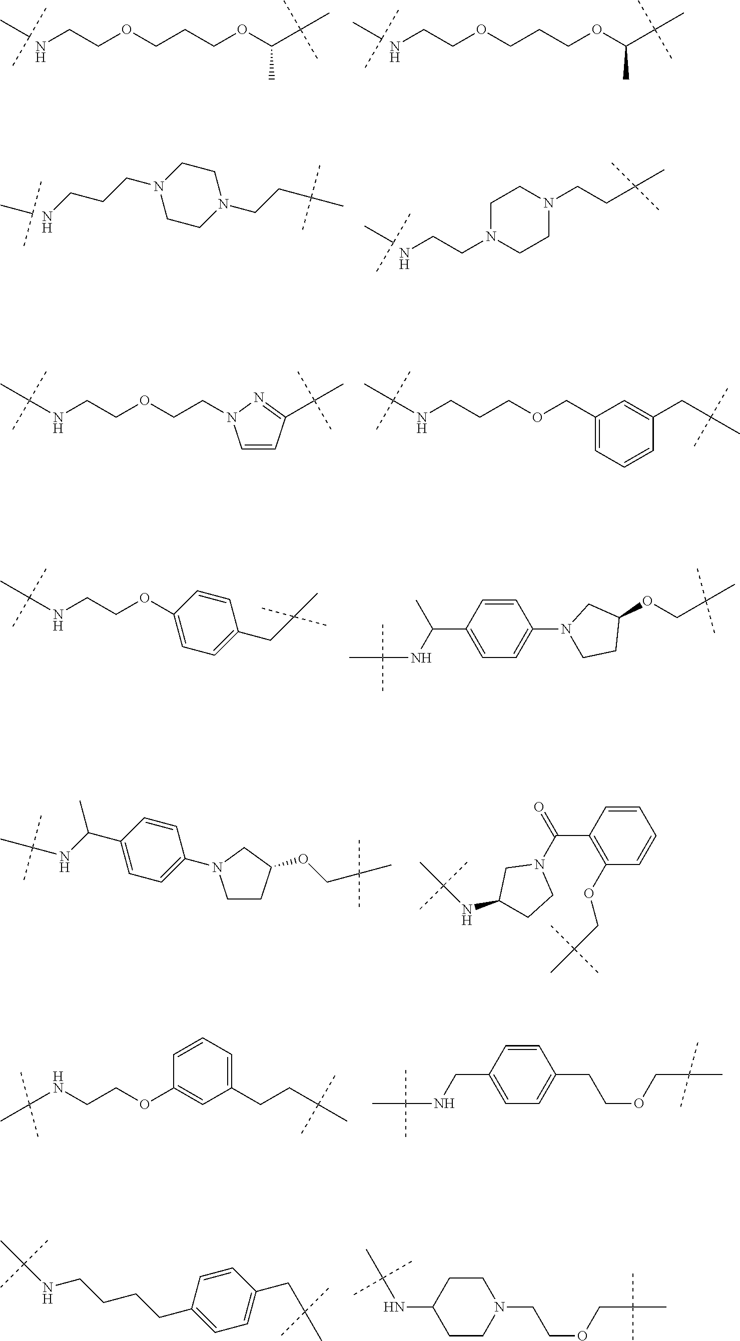

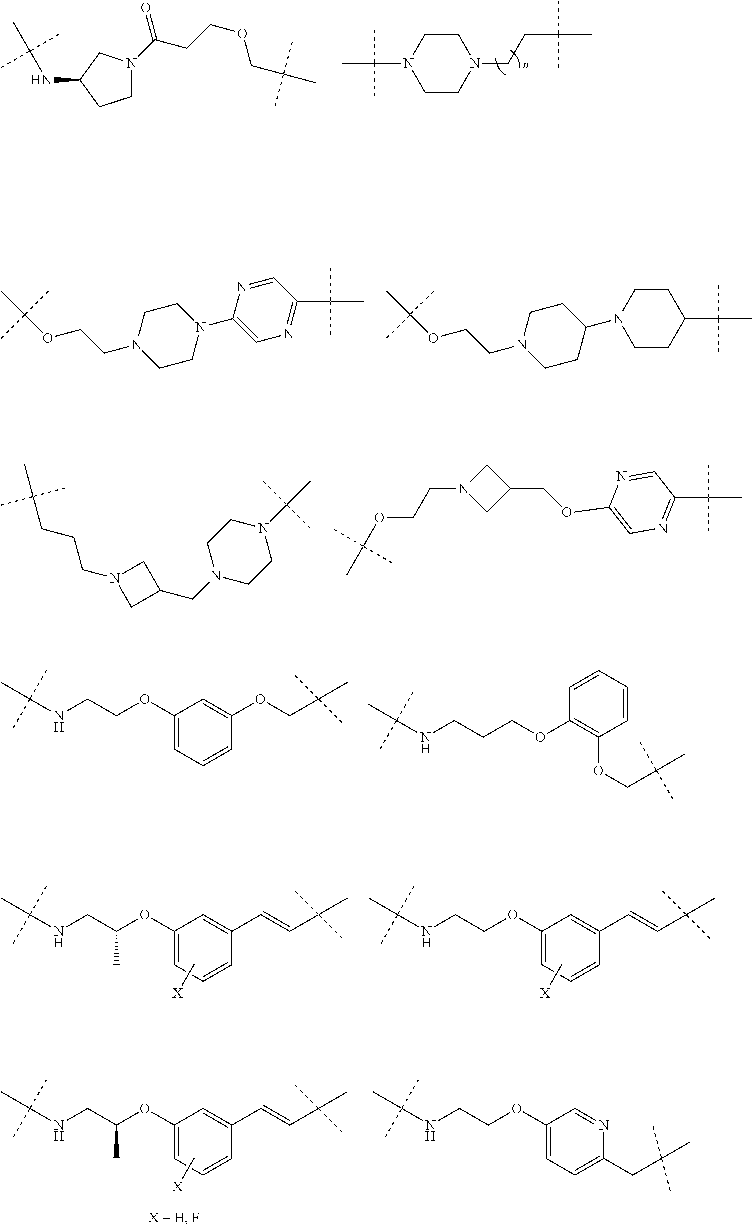

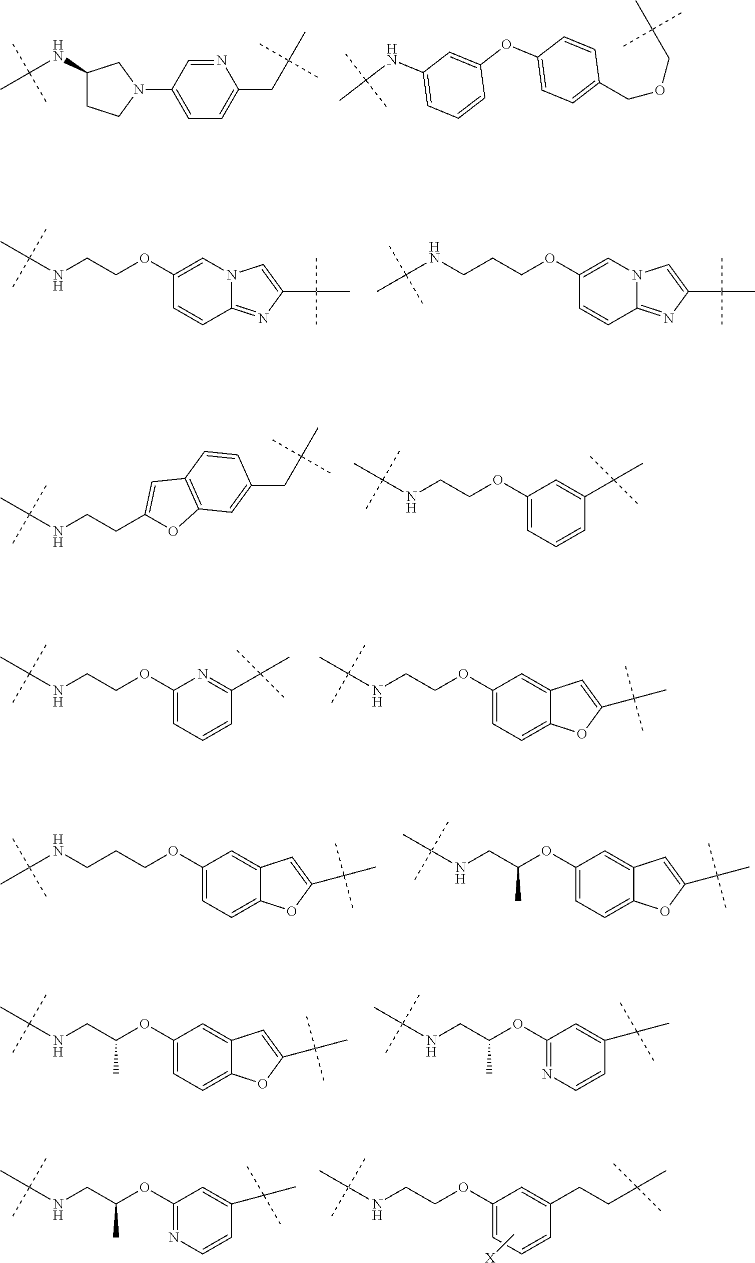

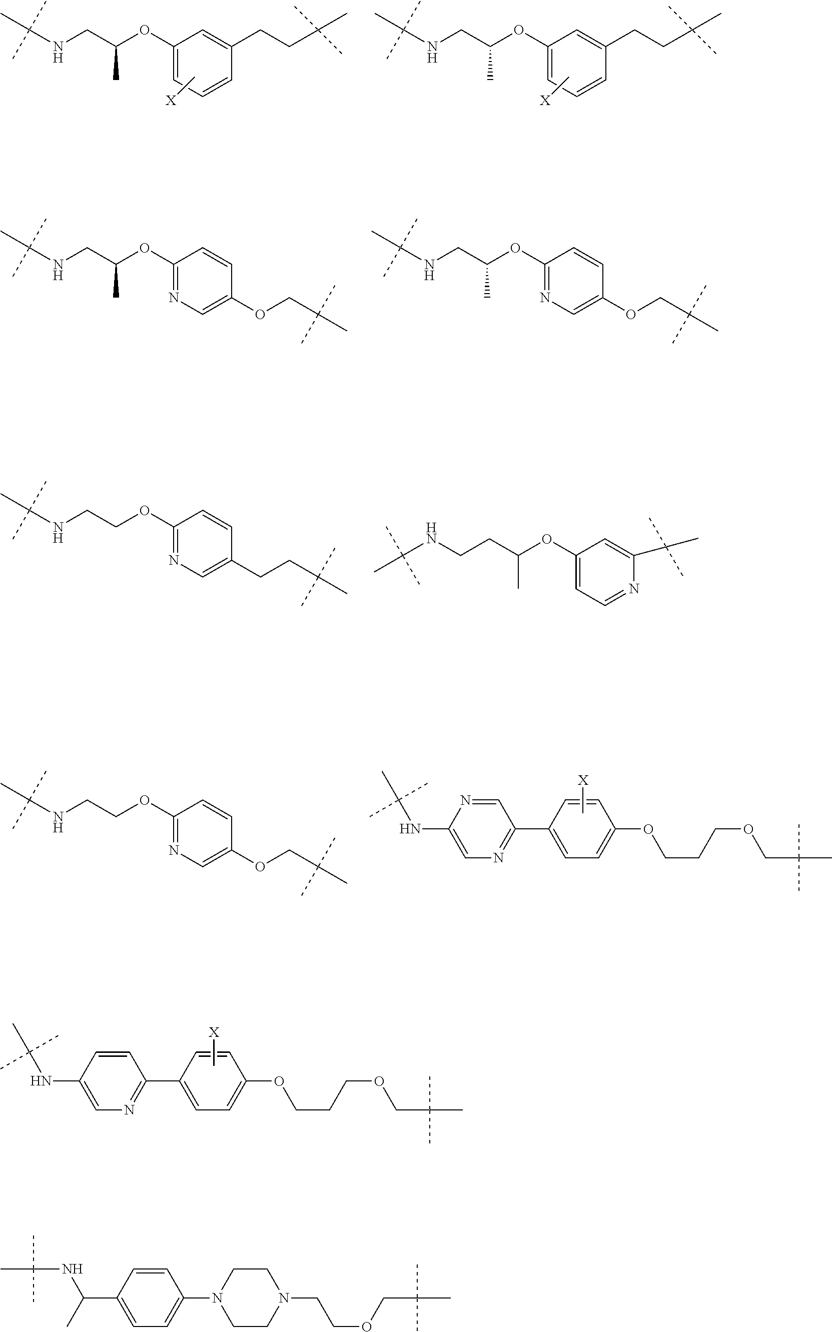

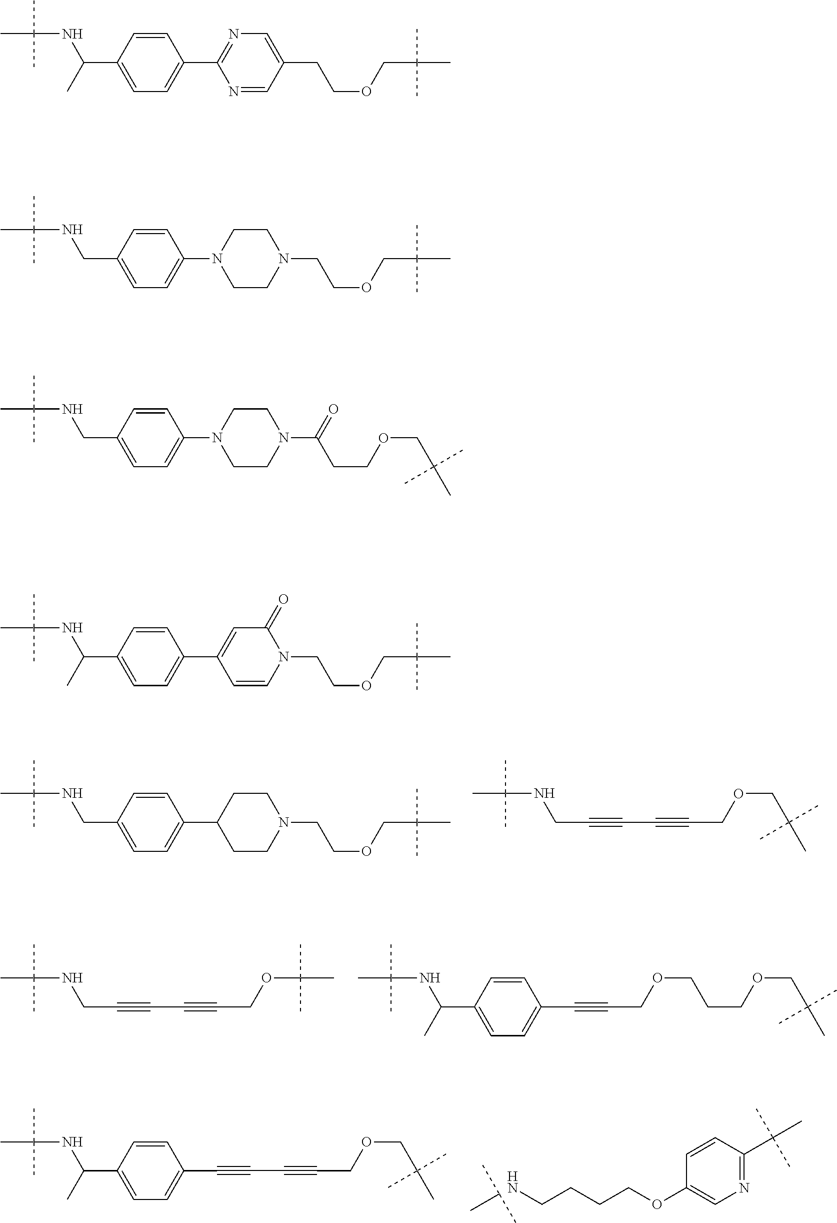

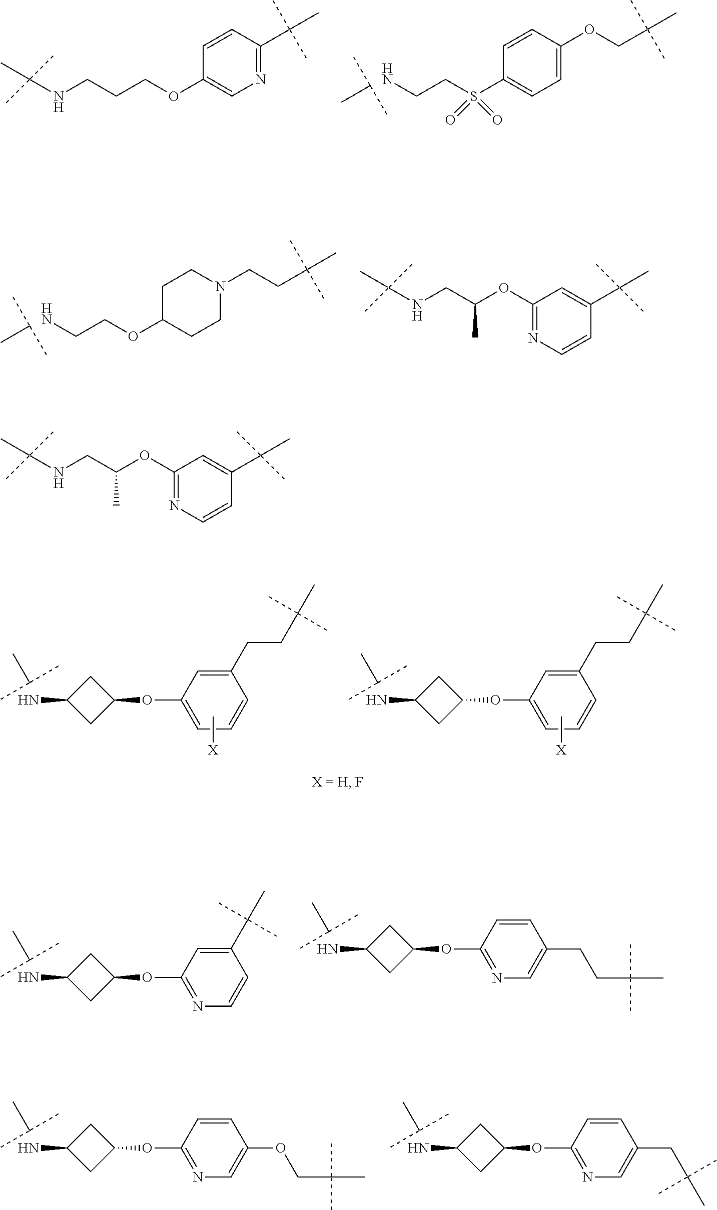

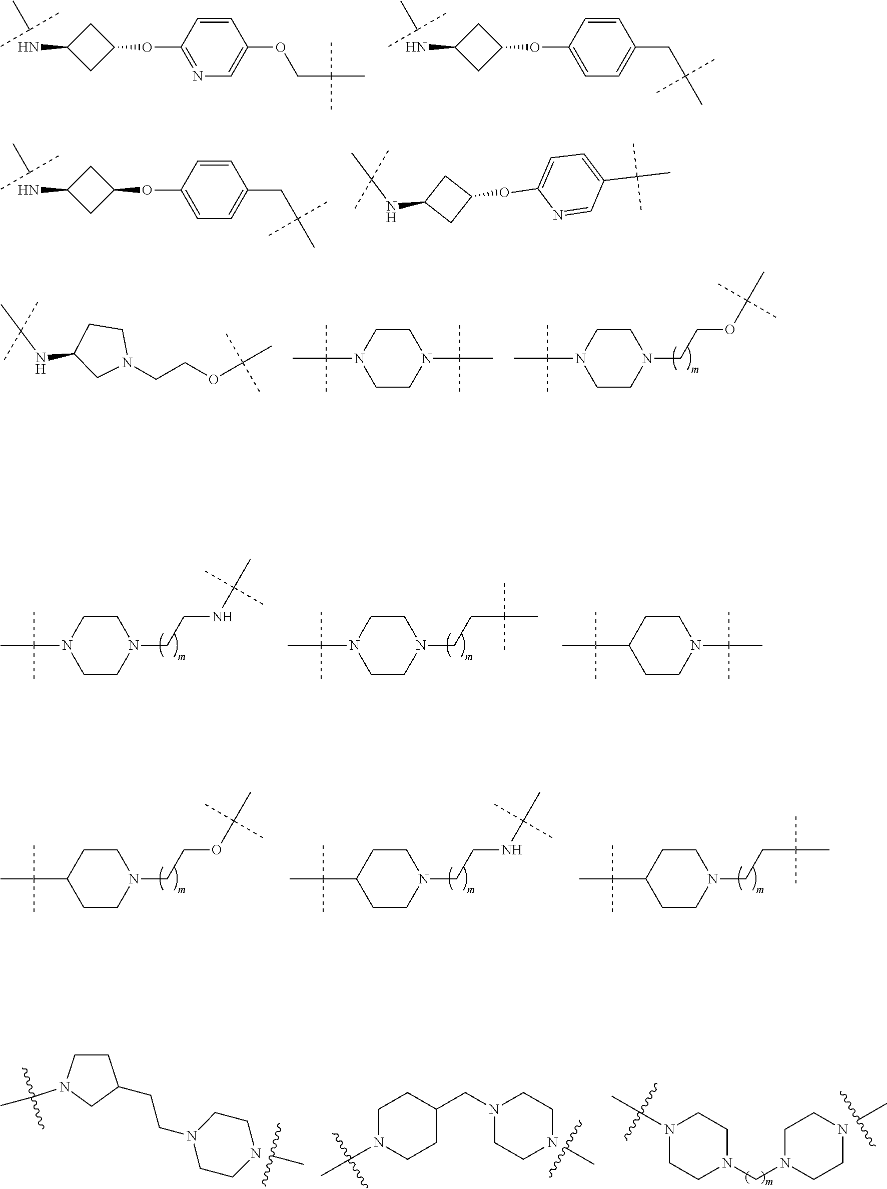

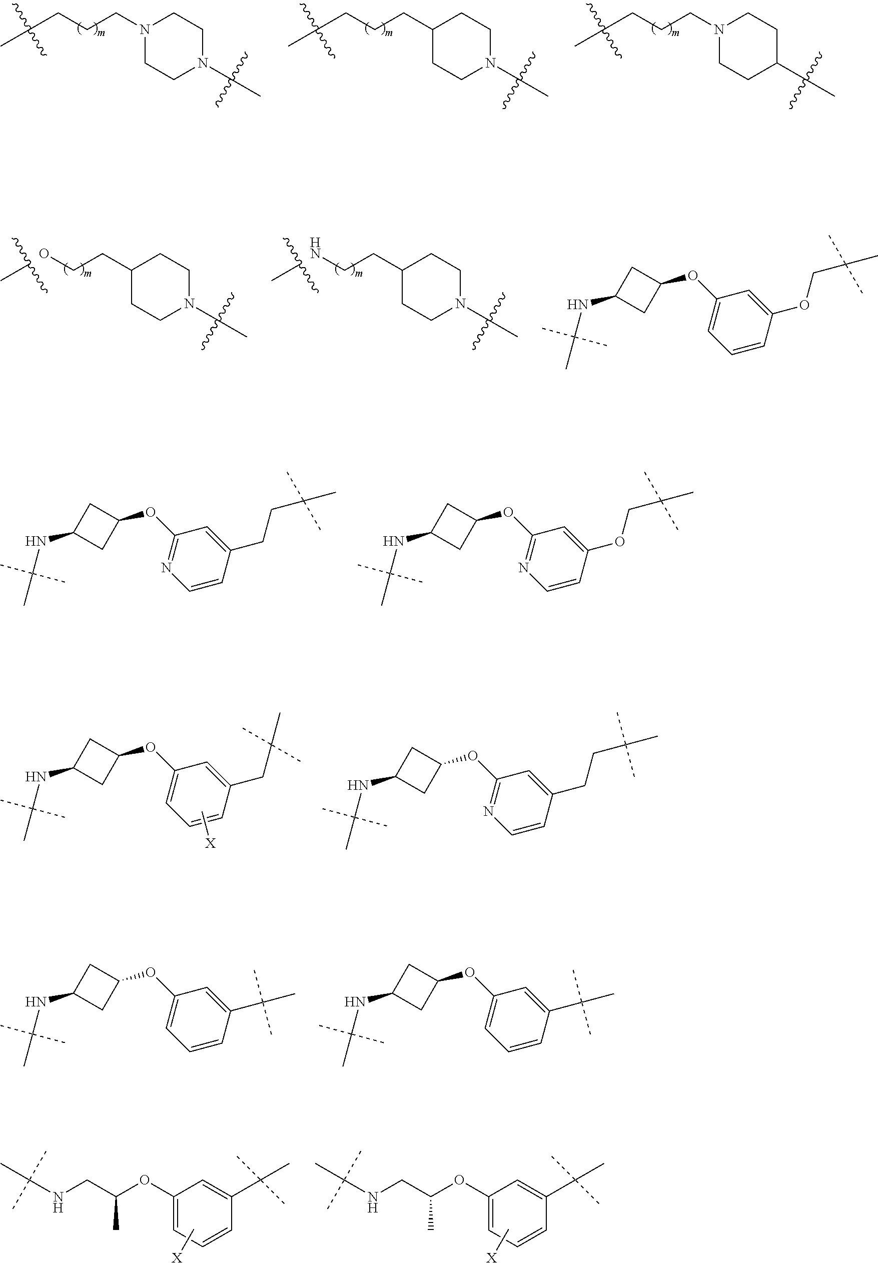

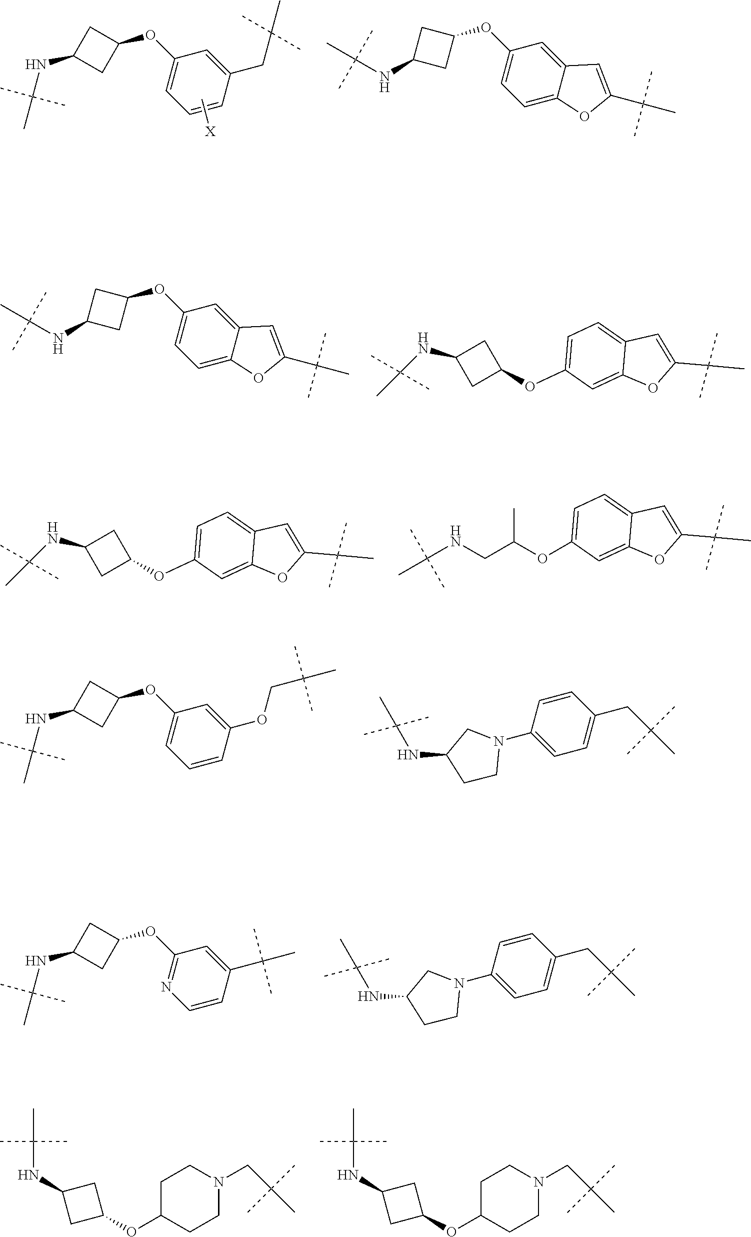

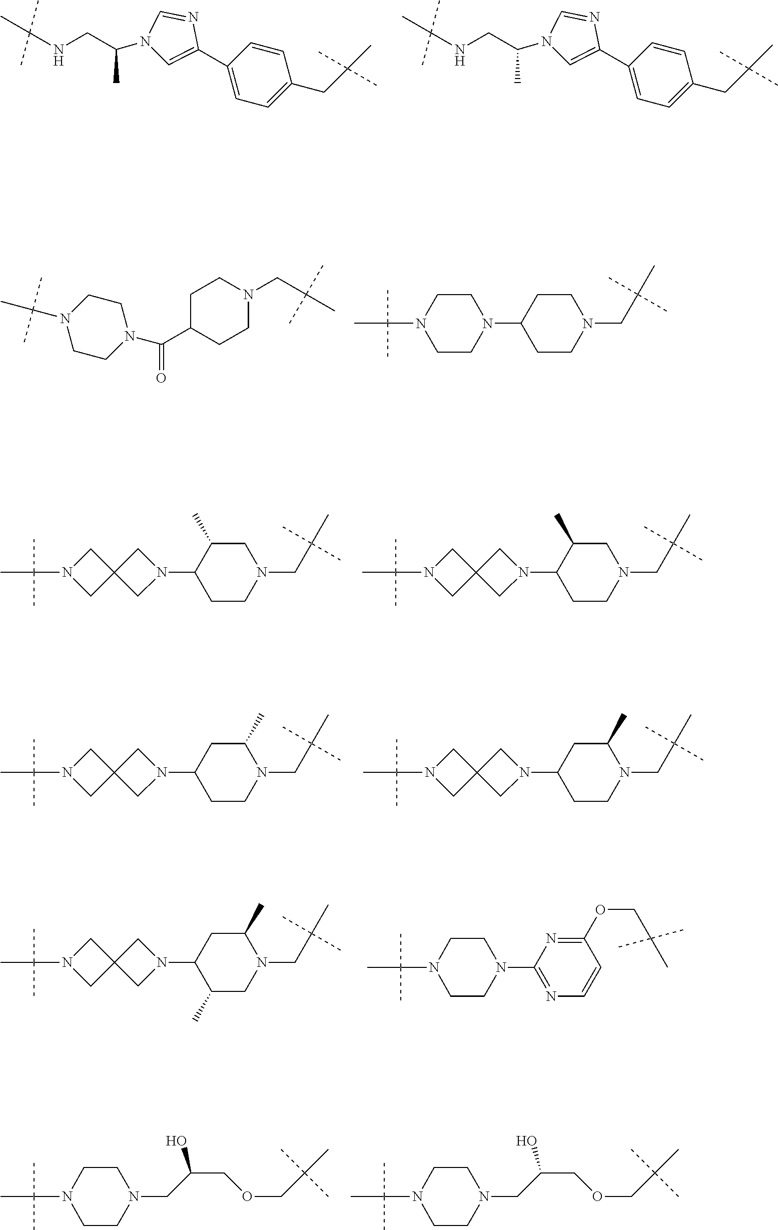

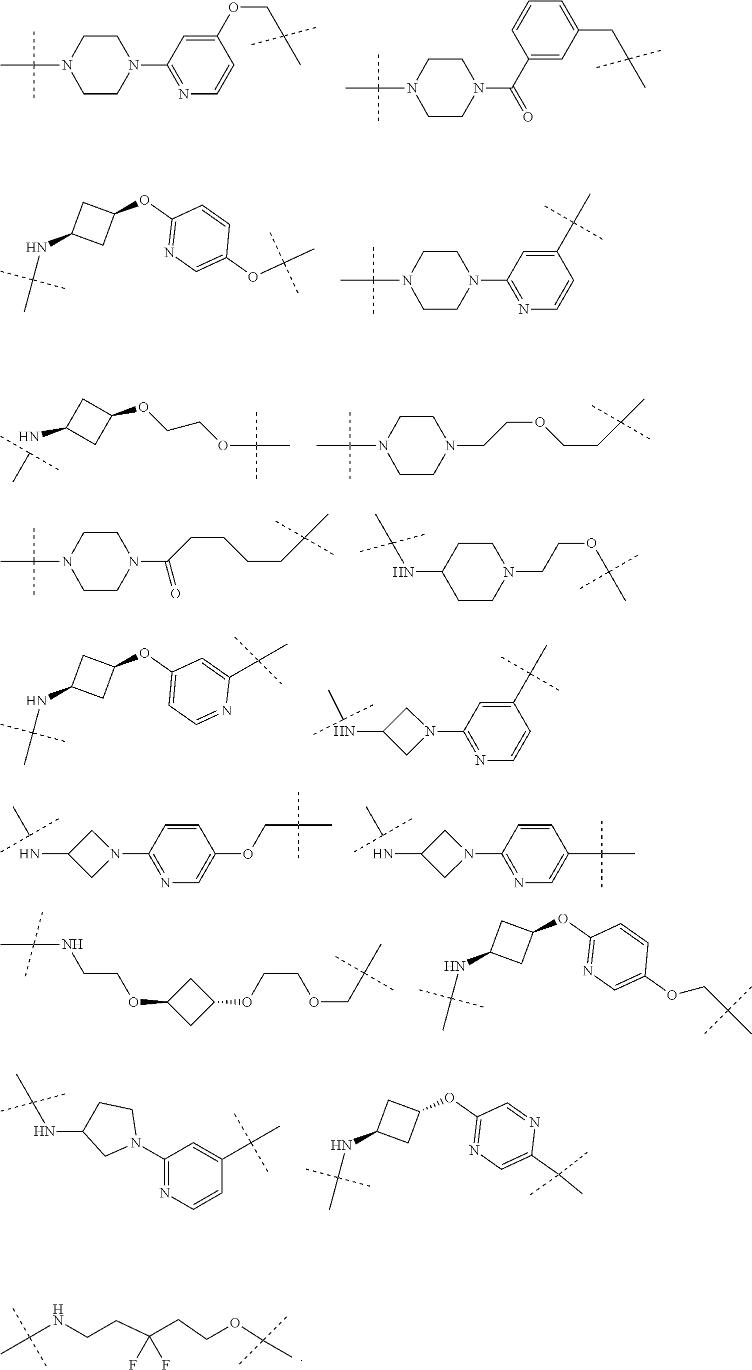

17. The compound according claim 15, wherein the linker (L) is selected from the group consisting of:

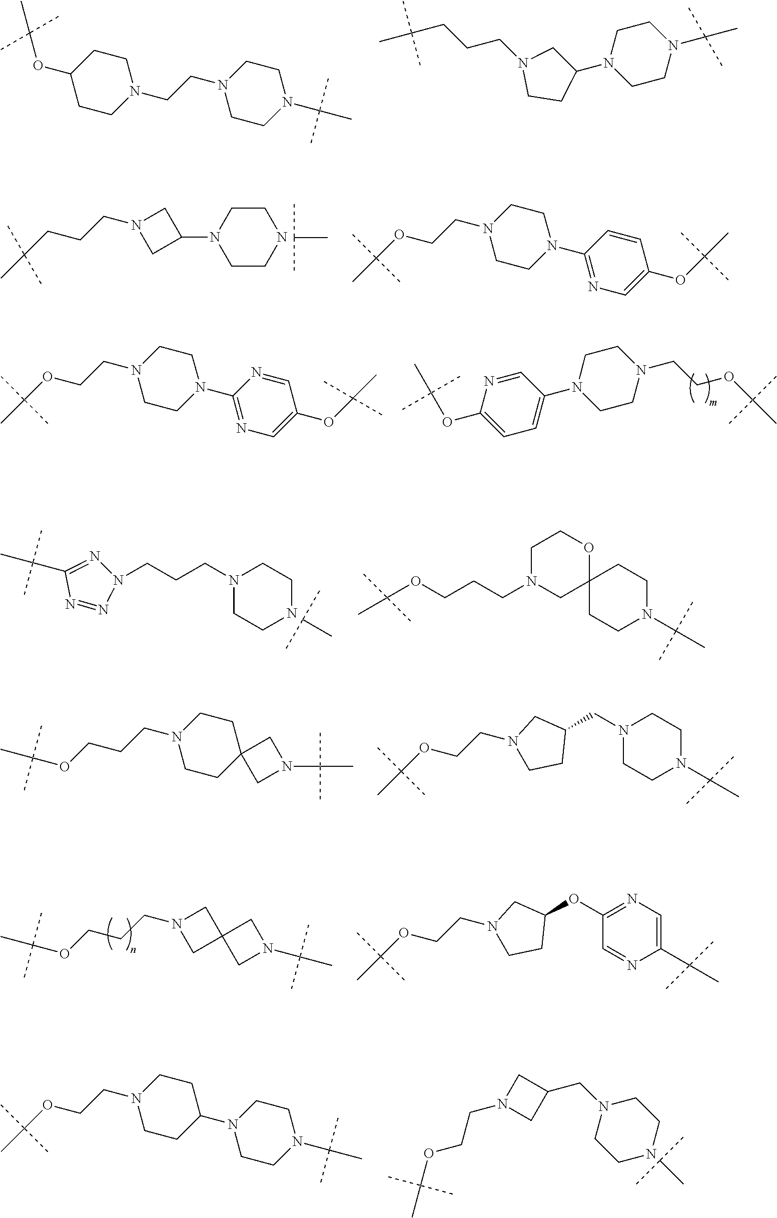

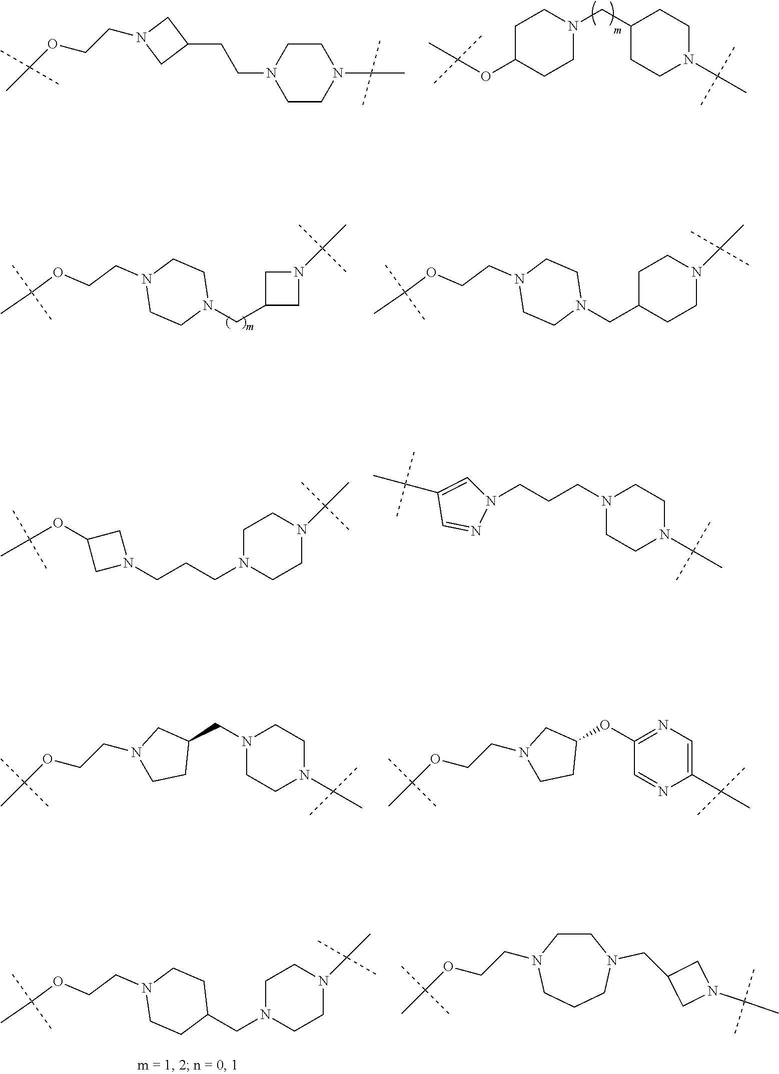

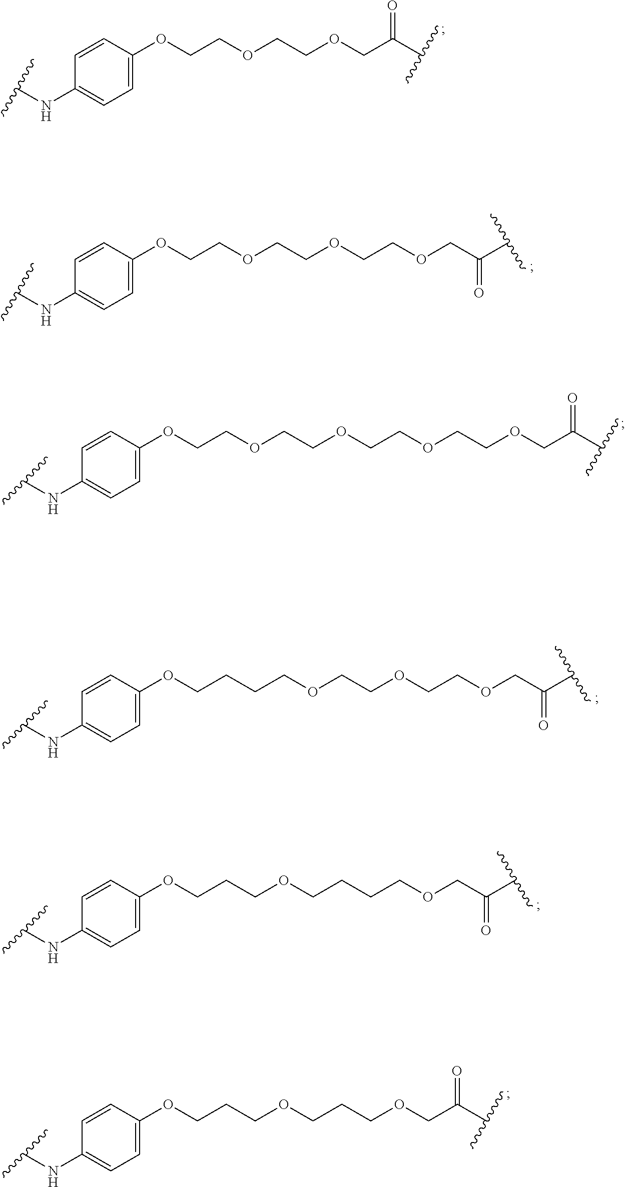 [SMILES: *Nc1ccc(OCCOCCOCC(*)=O)cc1 ; *Nc1ccc(OCCOCCOCCOCC(*)=O)cc1 ; *Nc1ccc(OCCOCCOCCOCCOCC(*)=O)cc1 ; *Nc1ccc(OCCCCOCCOCCOCC(*)=O)cc1 ; *Nc1ccc(OCCCOCCCCOCC(*)=O)cc1 ; *Nc1ccc(OCCCOCCCOCC(*)=O)cc1]

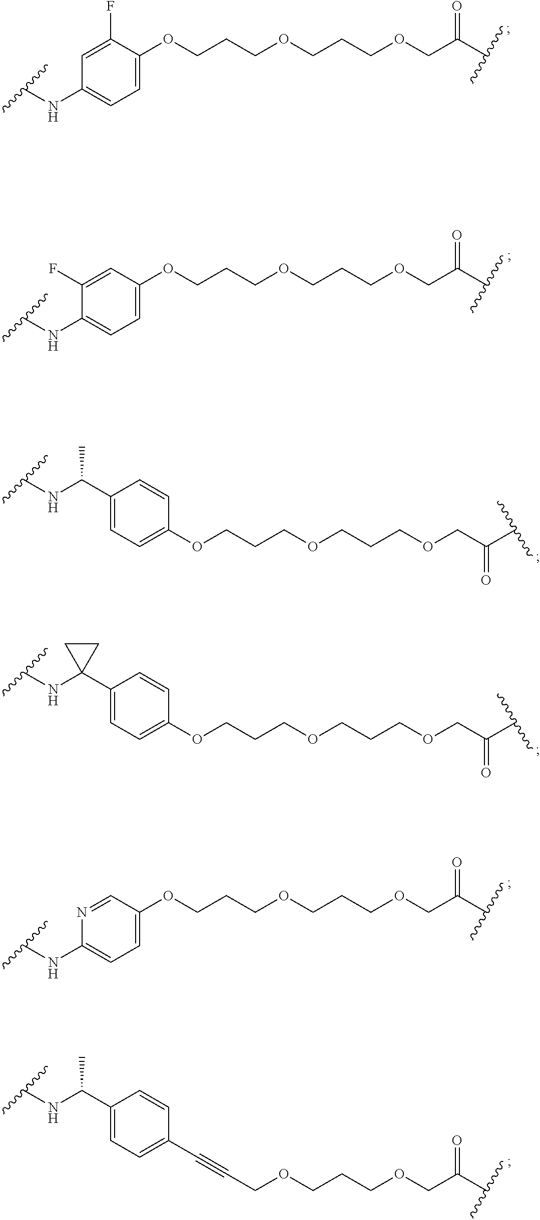 [SMILES: *Nc1ccc(OCCCOCCCOCC(*)=O)c(F)c1 ; *Nc1ccc(OCCCOCCCOCC(*)=O)cc1F ; *N[C@H](C)c1ccc(OCCCOCCCOCC(*)=O)cc1 ; *NC1(c2ccc(OCCCOCCCOCC(*)=O)cc2)CC1 ; *Nc1ccc(OCCCOCCCOCC(*)=O)cn1 ; *N[C@H](C)c1ccc(C#CCOCCCOCC(*)=O)cc1]

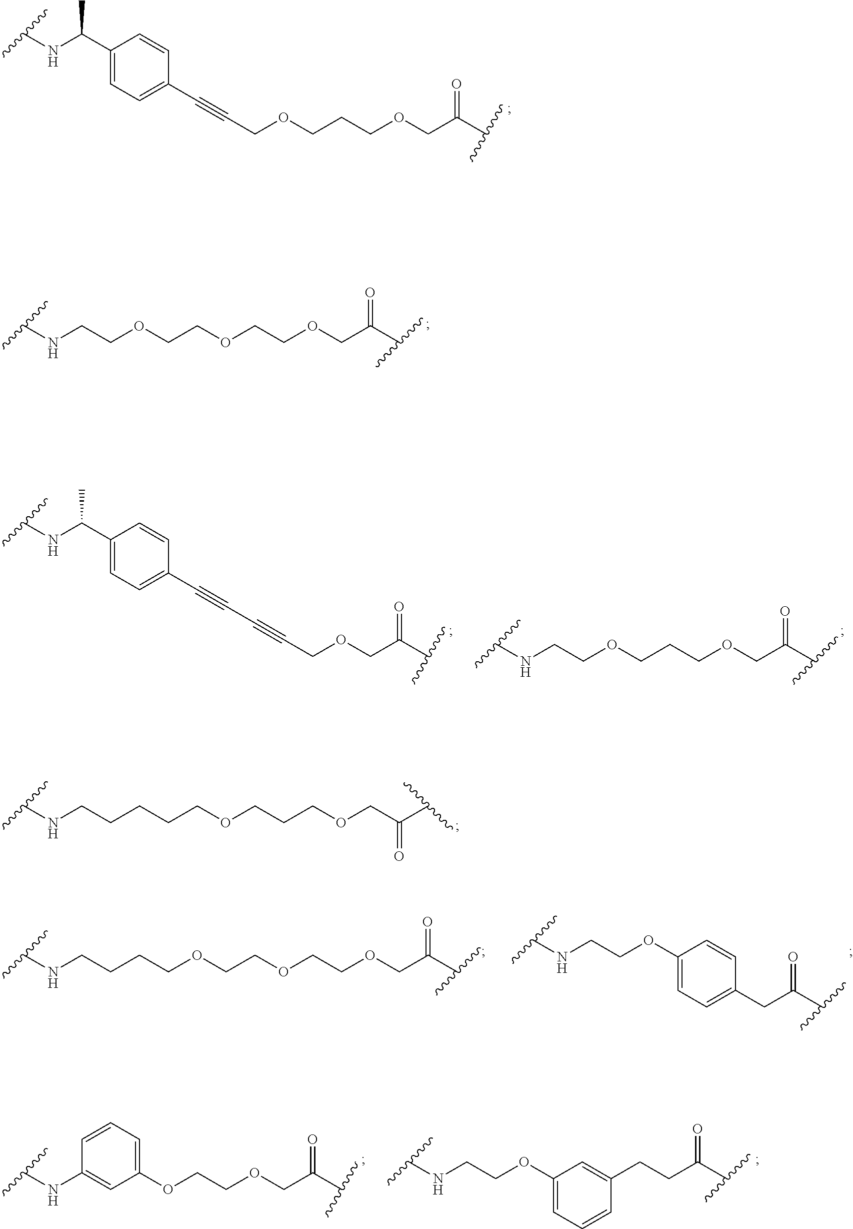

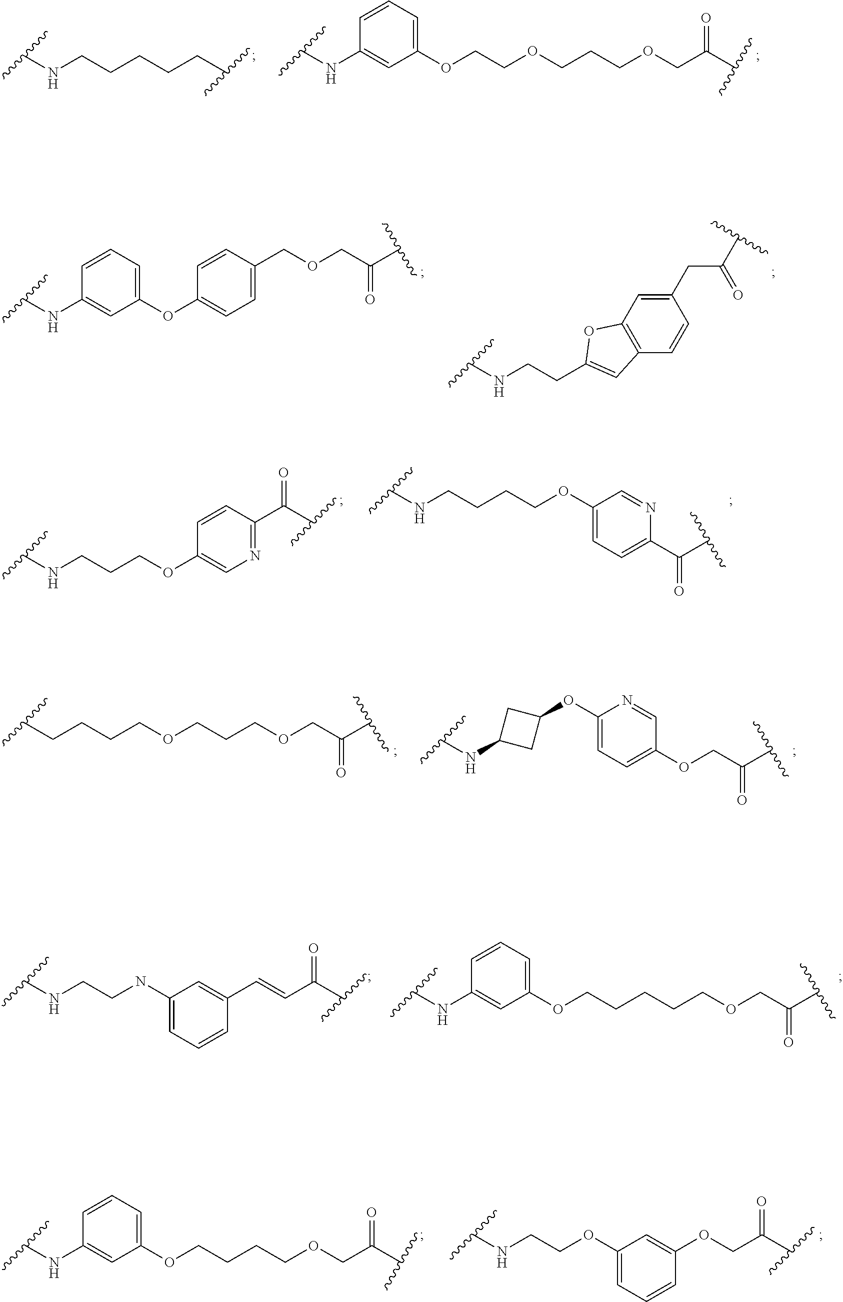

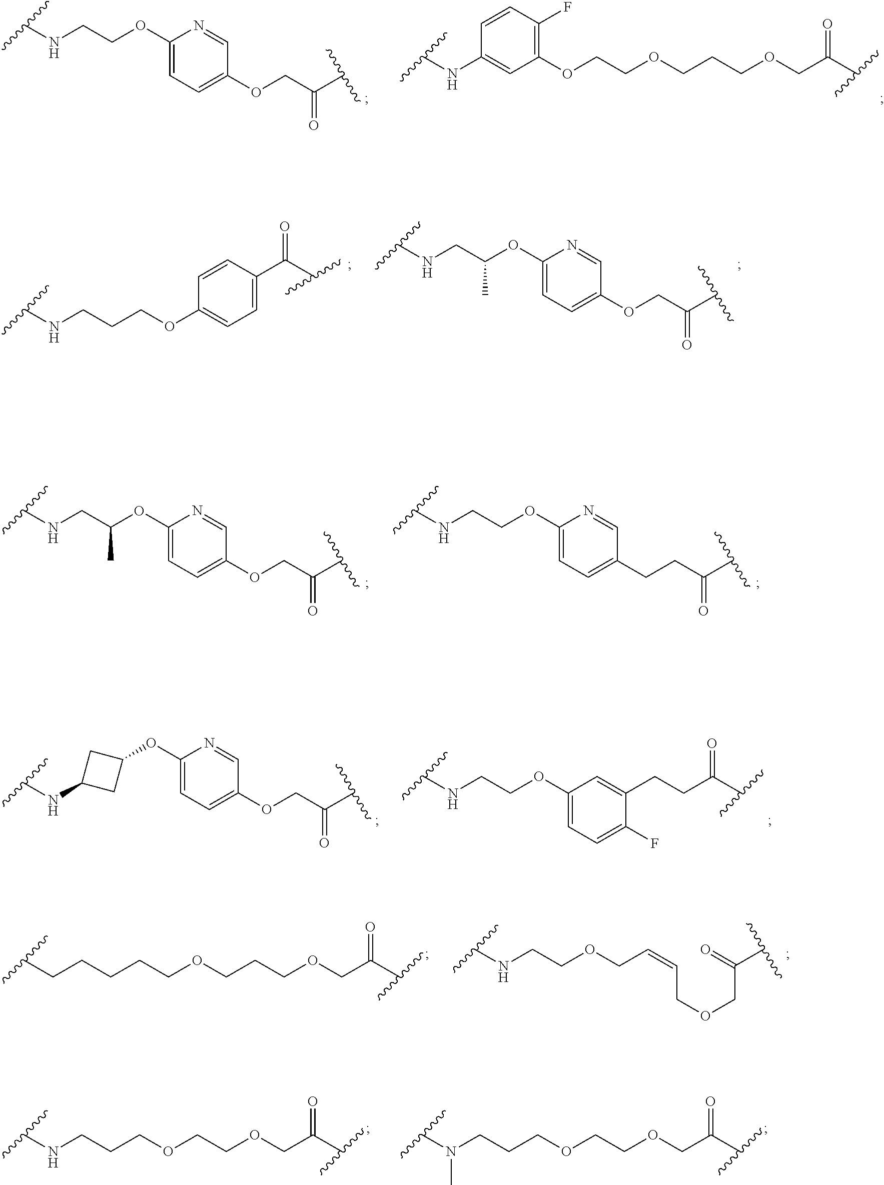

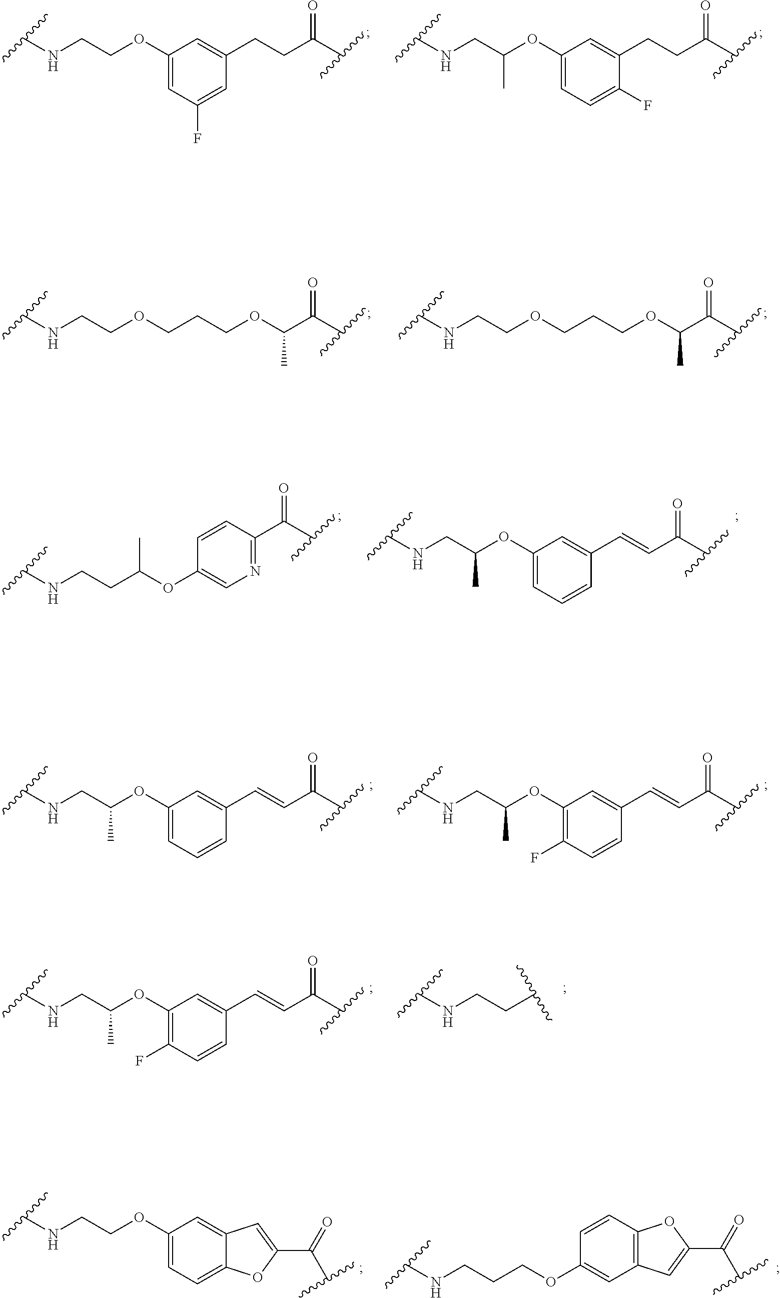

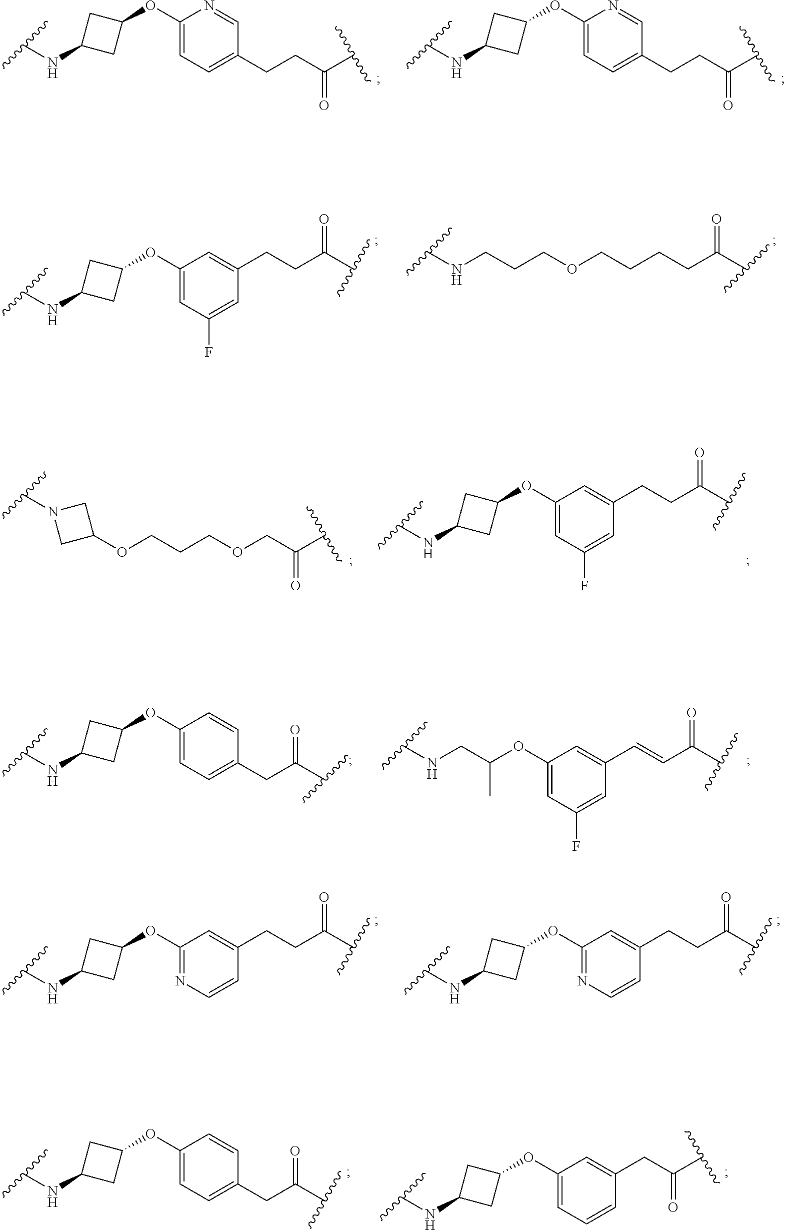

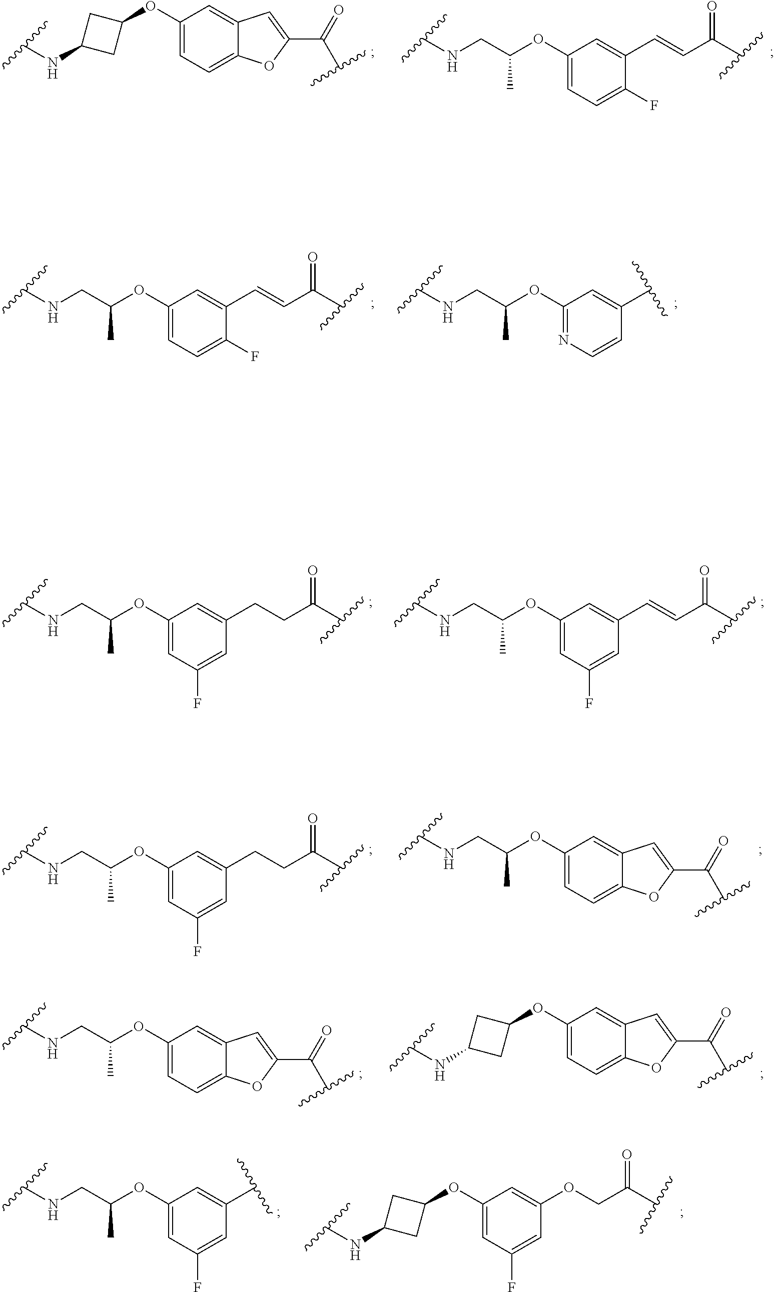

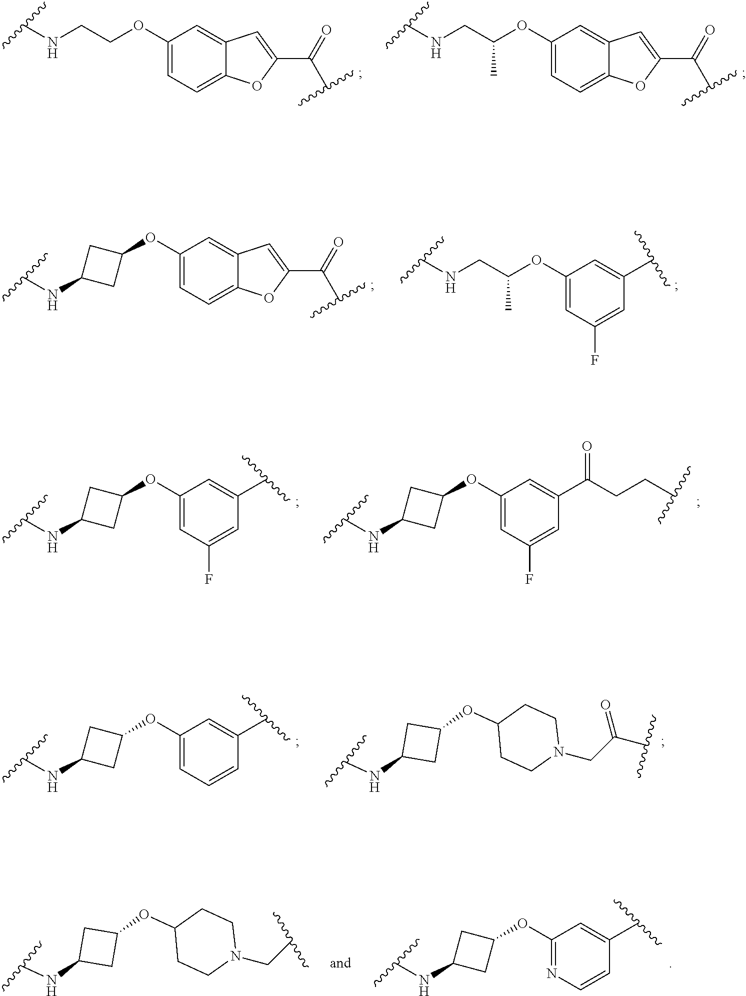

18. The compound according to claim 15, wherein the linker (L) is selected from the group consisting of:

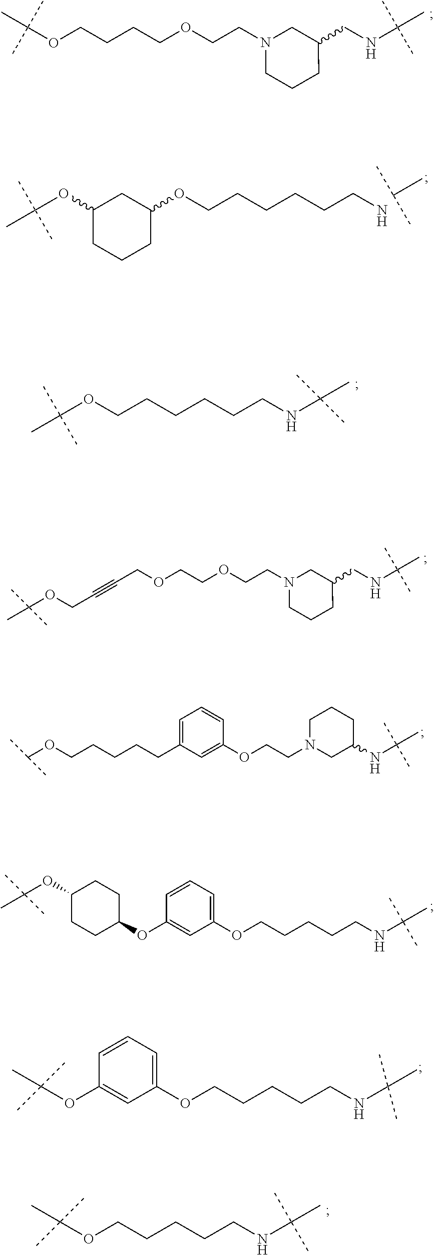

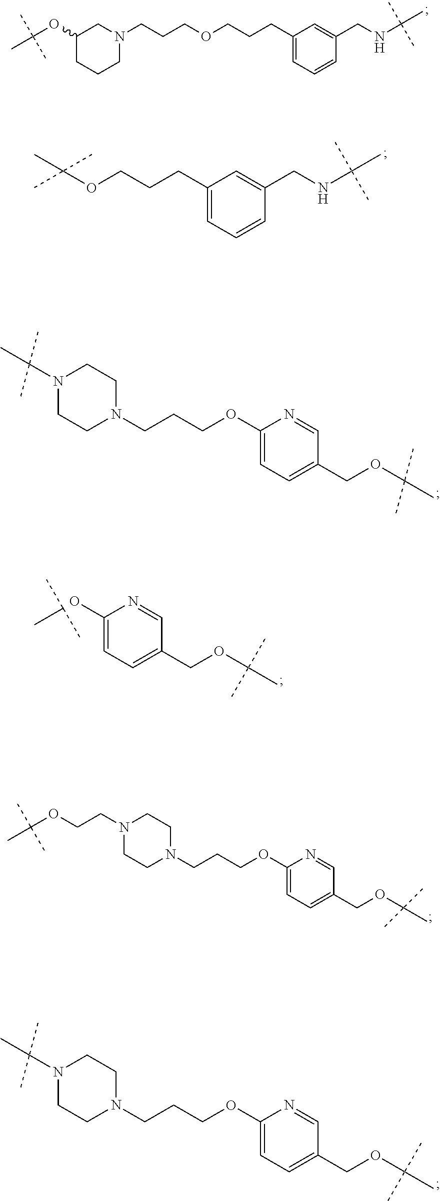

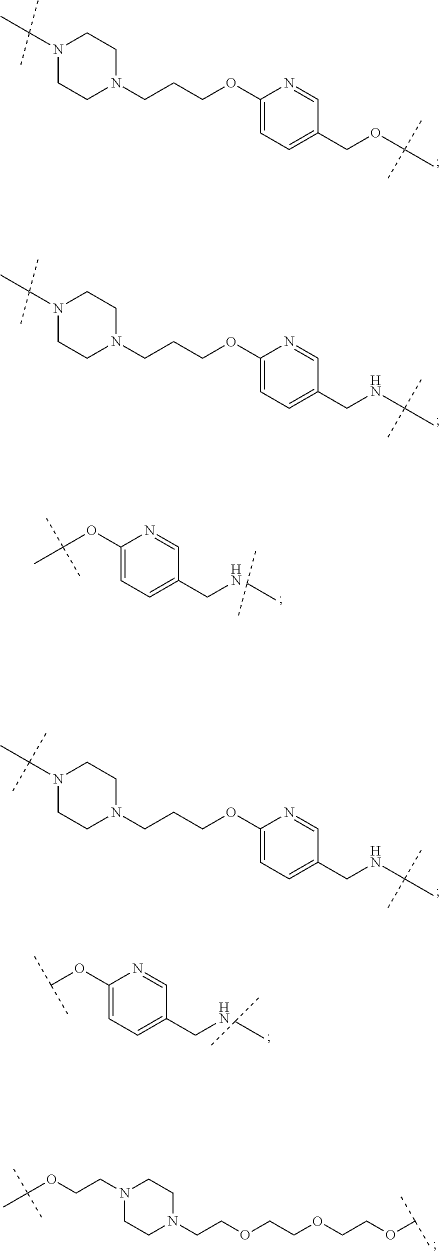

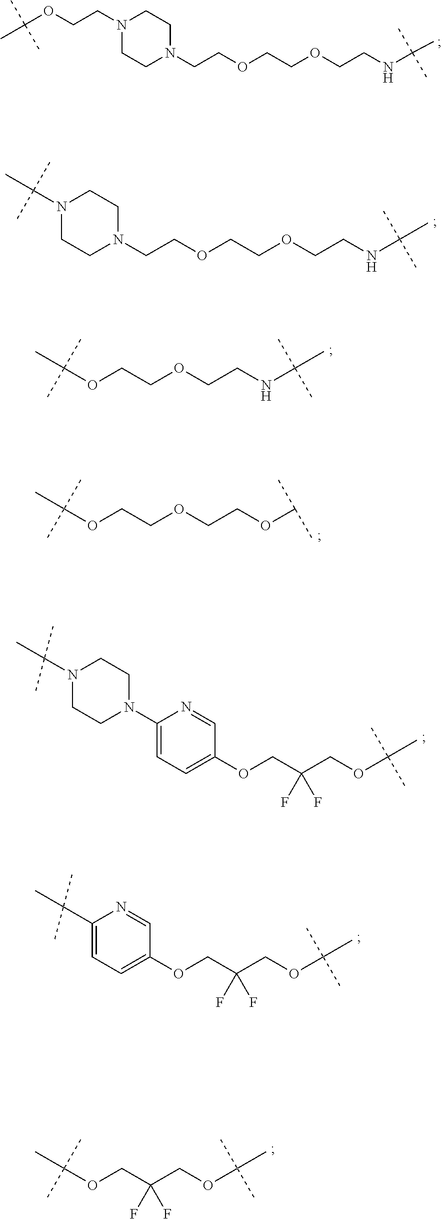

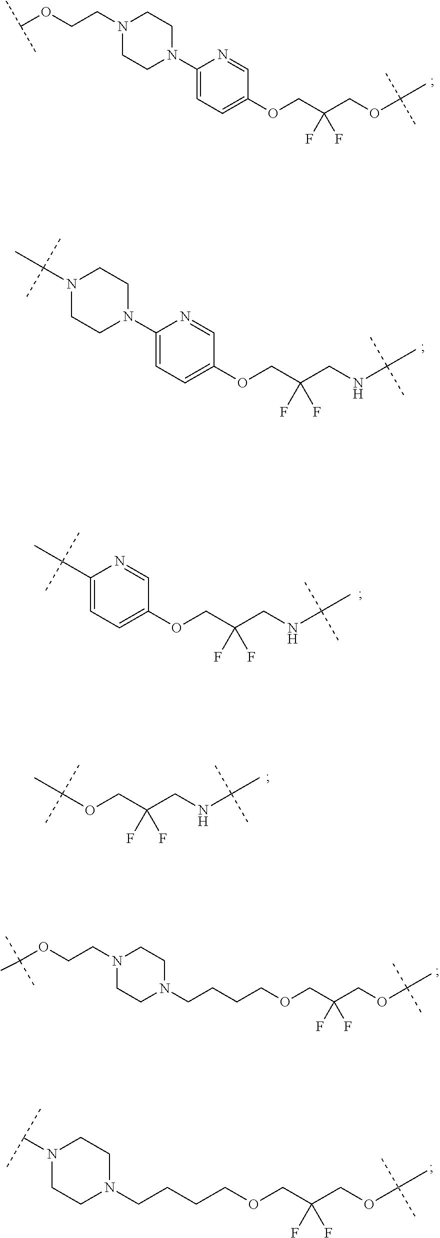

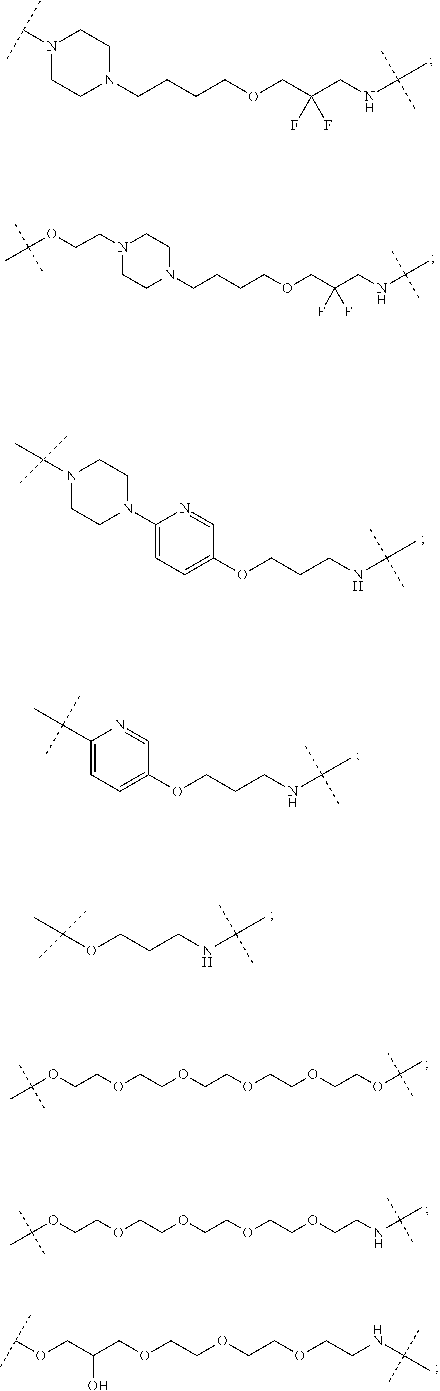

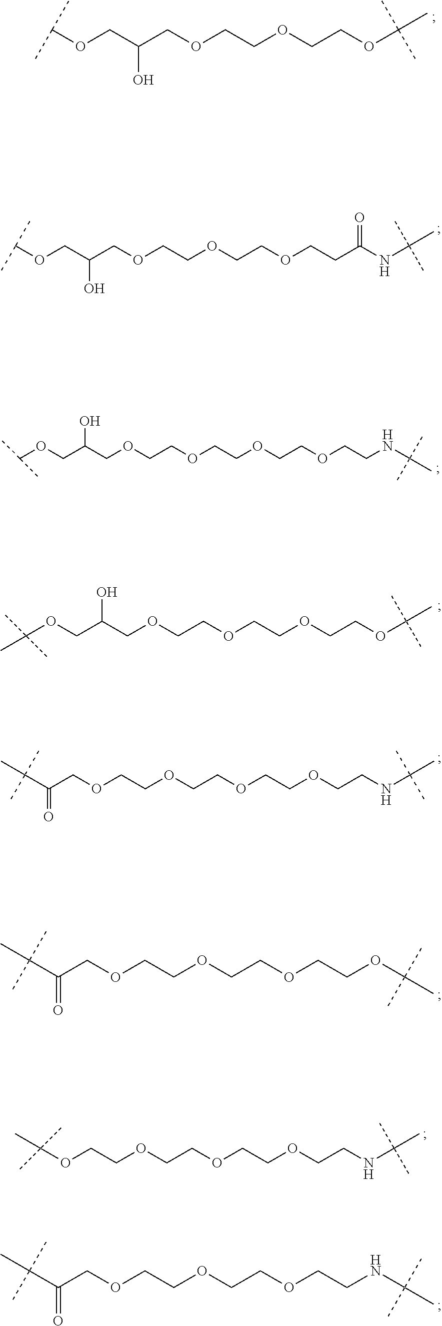

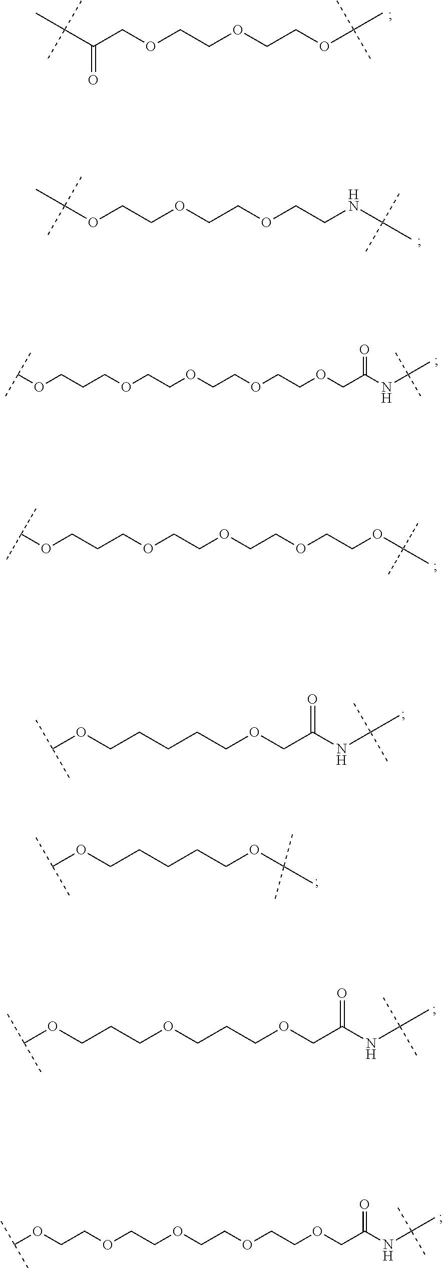

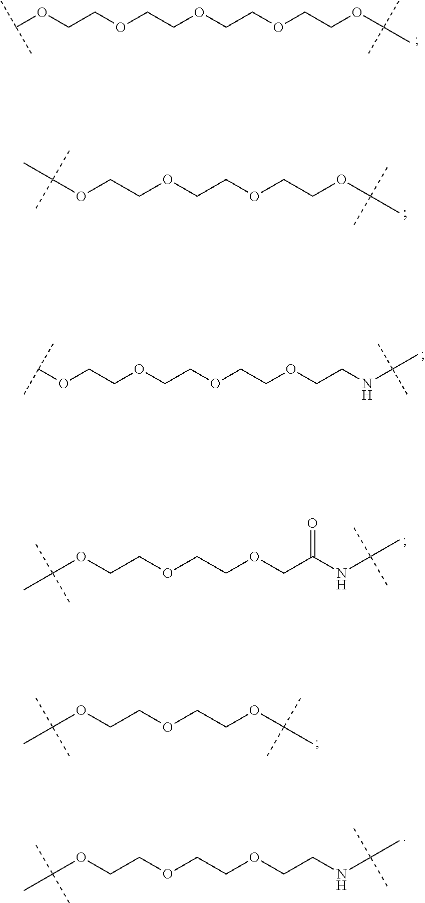

19. The compound according to claim 15, wherein the L is selected from: 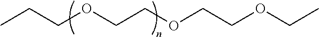
wherein n is an integer from 0 to 10; 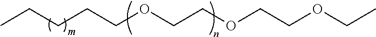
wherein n is an integer from 0 to 10, and m is an integer from 2 to 10; and 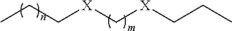
wherein n is an integer from 0 to 10, m is an integer from 0 to 10, and X is independently O or CH₂.

20. The compound according to claim 15, wherein the L is a polyethylenoxy group optionally substituted with aryl or phenyl comprising from 1 to 10 ethylene glycol units.

21. The compound according to claim 15, wherein the compound comprises multiple ULMs, multiple PTMs, multiple linkers or any combinations thereof.

22. The compound according to claim 1, wherein the compound is selected from the group consisting of compounds/examples 1-50.

23. A composition comprising an effective amount of a compound according to claim 1 and a pharmaceutically acceptable carrier.

24. The composition of claim 23, wherein the composition further comprises at least one of additional bioactive agent or another compound according to claim 1.

25. The composition of claim 24, wherein the additional bioactive agent is anti-cancer agent.

26. A composition comprising a pharmaceutically acceptable carrier and an effective amount of at least one compound according to claim 1 for treating a disease or disorder in a subject, the method comprising administering the composition to a subject in need thereof, wherein the compound is effective in treating or ameliorating at least one symptom of the disease or disorder.

27. The composition of claim 26, wherein the disease or disorder is associated with c-Met accumulation and aggregation.

28. The composition of claim 26, wherein the disease or disorder is cancer associated with c-Met accumulation and aggregation.

29. The composition of claim 26, wherein the disease or disorder is at least one of: gastric cancer, non-small cell lung cancer, squamous-cell carcinoma, basal cell carcinoma, adenocarcinoma, hepatocellular carcinomas, advanced hepatocellular carcinoma, renal cell carcinomas, and papillary renal cell carcinoma; cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas; T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML; or combinations thereof.

30. The composition of claim 26, wherein the disease or disorder is at least one of astric cancer, non-small cell lung cancer, advanced hepatocellular carcinoma (HCC), papillary renal cell cancer (RCC), or a combination thereof.

31. A composition comprising a pharmaceutically acceptable carrier and an effective amount of at least one compound of claim 1 for treating a disease or disorder associated with at least one of p38, p38α, p38β, p38γ, p38δ, or a combination thereof, the method comprising administering the composition to a subject in need thereof, wherein the compound is effective in treating or ameliorating at least one symptom of the disease or disorder and wherein:
the PTM is represented by the chemical structure: 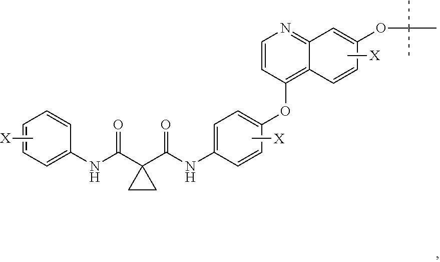
wherein each X is independently H, OMe, or F;
the linker (L) comprises a chemical structural unit represented by the formula:
-(A)_{q}-, wherein:
(A)_{q} is a group which is connected to a ULM or PTM moiety; and
q is an integer greater than or equal to 1;
each A is independently selected from the group consisting of, a bond, CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L2}, C≡C, siR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₃₋₁₁heteocyclyl optionally substituted with 0-6 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R^{L5} groups; and
R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cyclo N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl)SO₂N(C₁₋₈alkyl)₂, NH SO₂NH(C₁₋₈alkyl), NHSO₂N(C₁₋₈alkyl)₂, NHSO₂NH₂;
the ULM is represented by the chemical structure: 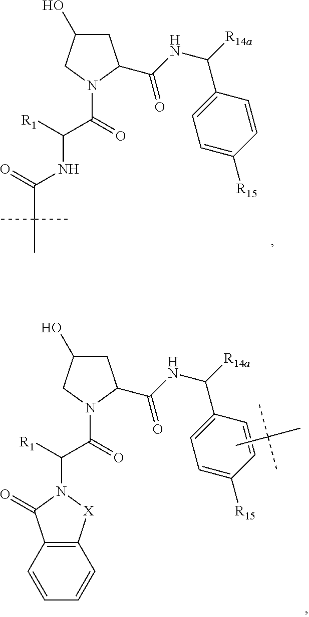
wherein
R₁ is H, ethyl, isopropyl, tert-butyl, sec-butyl, cyclopropyl, cyclobutyl, cyclopentyl, or cyclohexyl; optionally substituted alkyl, optionally substituted hydroxyalkyl, optionally substituted heteroaryl, or haloalkyl;
R₁₄ₐ is H, haloalkyl, optionally substituted alkyl, methyl, fluoromethyl, hydroxymethyl, ethyl, isopropyl, or cyclopropyl;
R₁₅ is selected from the group consisting of H, halogen, CN, OH, NO₂, optionally substituted heteroaryl, optionally substituted aryl; optionally substituted alkyl, optionally substituted haloalkyl, optionally substituted haloalkoxy, cycloalkyl, or cycloheteroalkyl (each optionally substituted);
X is C or C═O; and
the dashed line indicates the site of attachment to the linker.

32. The composition of claim 31, wherein the linker is an poly(alkylene) glycol of the structure —[O(CH₂)ₙ]ₘ—, wherein each n is independently 1, 2, 3, 4, 5, 6, 7 or 8, and m is an integer from 1-100 (e.g., 1, 2, 3, 4, 5, 6, 7, 8, 9, or 10).

33. The composition of claim 31, wherein the linker is selected from: 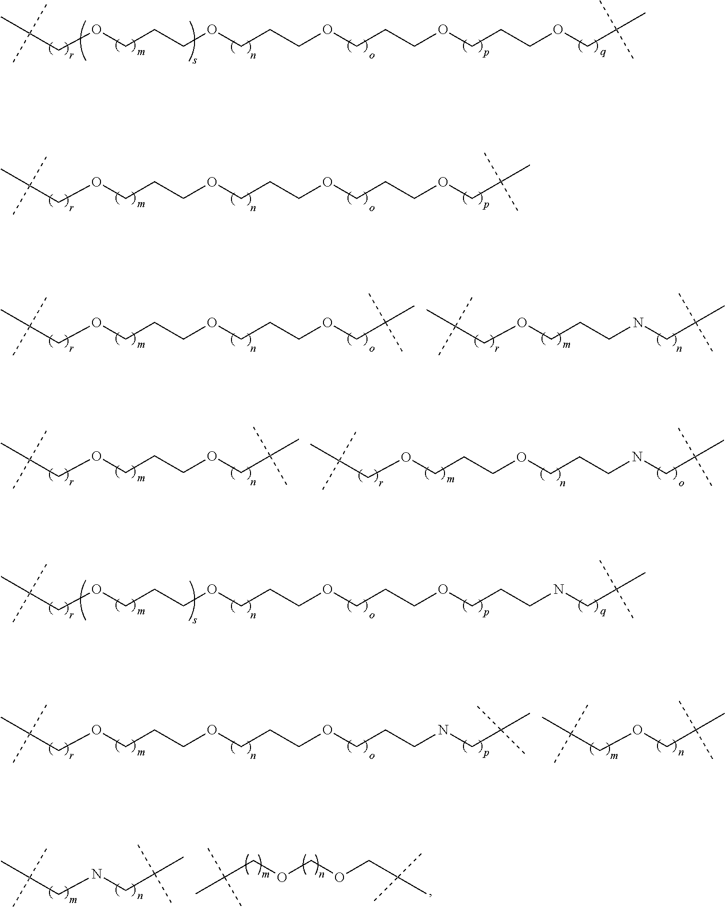
wherein each m, n, o, p, q, r, and s is independently 0, 1, 2, 3, 4, 5, 6, 7, 8, 9, 10, 11, 12, 13, 14, 15, 16, 17, 18, 19, or 20

34. The composition of claim 31, wherein the linker is selected from the group consisting of:

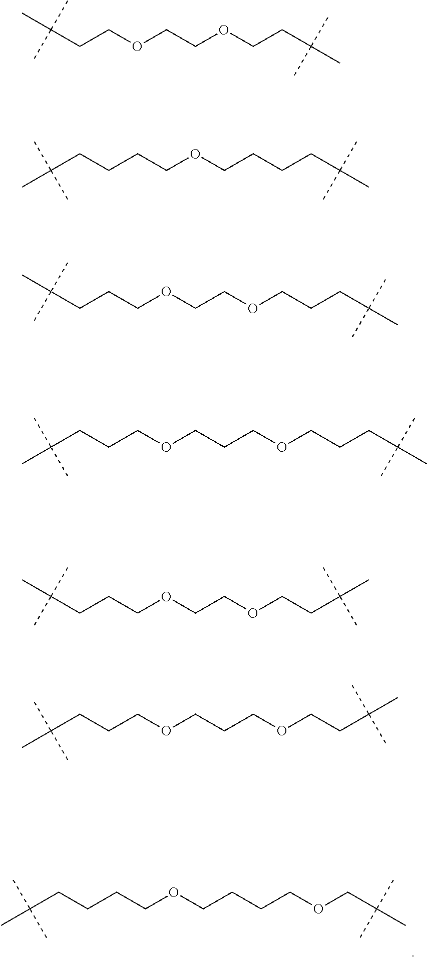

35. The composition of claim 31, wherein the linker is 9-14 atoms in length.

36. The composition of claim 31, wherein the disease or disorder is at least one of an autoimmune or inflammatory disease or disorder, cancer, cardiovascular disease or disorder, a neurological disease or disorder, or a combination thereof.

37. The composition of claim 31, wherein:
the autoimmune disease or disorder is at least one of: rheumatoid arthritis, cerebral ischemia, muscular dystrophy, diabetes mellitus, Crohn's disease, psoriasis, ankylosing spondylitis, chronic asthma, chronic pulmonary obstructive disorder, or a combination thereof;
the cancer is at least one of: gastric cancer, non-small cell lung cancer, squamous-cell carcinoma, basal cell carcinoma, adenocarcinoma, hepatocellular carcinomas, advanced hepatocellular carcinoma, renal cell carcinomas, and papillary renal cell carcinoma;
cancer of the bladder, bowel, breast, cervix, colon, esophagus, head, kidney, liver, lung, neck, ovary, pancreas, prostate, and stomach; leukemia; benign and malignant lymphomas, particularly Burkitt's lymphoma and Non-Hodgkin's lymphoma; myeloma; multiple myeloma, benign and malignant melanomas; myeloproliferative diseases; sarcomas, Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas; T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, B-cell chronic lymphatic leukemia, Philadelphia chromosome positive ALL, Philadelphia chromosome positive CML; or combinations thereof;
the cardiovascular disease or disorder is at least one of: ischemia, ischemia-reperfusion, or a combination thereof;
the neurological disease or disorder if at least one of: Parkinson's disease, Alzheimer's disease, multiple sclerosis, amyotrophic lateral sclerosis (ALS), chronic inflammatory demyelinating polyradiculoneuropathy, fibromyalgia, polymyositis, or a combination thereof; or
a combination thereof.

38. The composition of claim 31, wherein the compound is selected from the group consisting of compound 1, 46, 48, 49, and 50.
